SEQUENCE LISTING (1359 sequences)

SEQ ID NO: 1 - DNA, 346 bp, Homo sapiens
  source 1..346: mol_type = other DNA
gatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagcagaggcccgggggcctcggaagatacgcagtattttggcccaggcacccggctgacagtgctcg

SEQ ID NO: 2 - protein, 115 aa, Homo sapiens
DAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSRGPGASEDTQYFGPGTRLTVL

SEQ ID NO: 3 - protein, 5 aa, Homo sapiens
LNHNV

SEQ ID NO: 4 - protein, 6 aa, Homo sapiens
YYDKDF

SEQ ID NO: 5 - protein, 16 aa, Homo sapiens
CATSRGPGASEDTQYF

SEQ ID NO: 6 - protein, 26 aa, Homo sapiens
DAMVIQNPRYQVTQFGKPVTLSCSQT

SEQ ID NO: 7 - protein, 17 aa, Homo sapiens
MYWYQQKSSQAPKLLFH

SEQ ID NO: 8 - protein, 36 aa, Homo sapiens
NNEADTPDNFQSRRPNTSFCFLDIRSPGLGDAAMYL

SEQ ID NO: 9 - protein, 9 aa, Homo sapiens
GPGTRLTVL

SEQ ID NO: 10 - DNA, 331 bp, Homo sapiens
  source 1..331: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaatgagtatggaaacaaactggtctttggcgcaggaaccattctgagagtcaagtcct

SEQ ID NO: 11 - protein, 110 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVNEYGNKLVFGAGTILRVKS

SEQ ID NO: 12 - protein, 6 aa, Homo sapiens
DRGSQS

SEQ ID NO: 13 - protein, 6 aa, Homo sapiens
IYSNGD

SEQ ID NO: 14 - protein, 12 aa, Homo sapiens
CAVNEYGNKLVF

SEQ ID NO: 15 - protein, 26 aa, Homo sapiens
QKEVEQNSGPLSVPEGAIASLNCTYS

SEQ ID NO: 16 - protein, 17 aa, Homo sapiens
FFWYRQYSGKSPELIMF

SEQ ID NO: 17 - protein, 33 aa, Homo sapiens
KEDGRFTAQLNKASQYVSLLIRDSQPSDSATYL

SEQ ID NO: 18 - protein, 10 aa, Homo sapiens
GAGTILRVKS

SEQ ID NO: 19 - DNA, 340 bp, Homo sapiens
  source 1..340: mol_type = other DNA
gatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatctaagtatcaatcagccccagcattttggtgatgggactcgactctccatcctag

SEQ ID NO: 20 - protein, 113 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFCATSDLSINQPQHFGDGTRLSIL

SEQ ID NO: 21 - protein, 5 aa, Homo sapiens
KGHDR

SEQ ID NO: 22 - protein, 6 aa, Homo sapiens
SFDVKD

SEQ ID NO: 23 - protein, 14 aa, Homo sapiens
CATSDLSINQPQHF

SEQ ID NO: 24 - protein, 26 aa, Homo sapiens
DADVTQTPRNRITKTGKRIMLECSQT

SEQ ID NO: 25 - protein, 17 aa, Homo sapiens
MYWYRQDPGLGLRLIYY

SEQ ID NO: 26 - protein, 36 aa, Homo sapiens
INKGEISDGYSVSRQAQAKFSLSLESAIPNQTALYF

SEQ ID NO: 27 - protein, 9 aa, Homo sapiens
GDGTRLSIL

SEQ ID NO: 28 - DNA, 328 bp, Homo sapiens
  source 1..328: mol_type = other DNA
caacaaccagtgcagagtcctcaagccgtgatcctccgagaaggggaagatgctgtcatcaactgcagttcctccaaggctttatattctgtacactggtacaggcagaagcatggtgaagcacccatcttcctgatgatattactgaagggtggagaacagaagggtcatgacaaaatatctgcttcatttaatgaaaaaaagcagcaaagctccctgtaccttacggcctcccagctcagttactcaggaacctacttctgtggcacagagatagggaccgacaagctcatctttgggactgggaccagattacaagtctttccaa

SEQ ID NO: 29 - protein, 109 aa, Homo sapiens
QQPVQSPQAVILREGEDAVINCSSSKALYSVHWYRQKHGEAPIFLMILLKGGEQKGHDKISASFNEKKQQSSLYLTASQLSYSGTYFCGTEIGTDKLIFGTGTRLQVFP

SEQ ID NO: 30 - protein, 5 aa, Homo sapiens
KALYS

SEQ ID NO: 31 - protein, 7 aa, Homo sapiens
LLKGGEQ

SEQ ID NO: 32 - protein, 12 aa, Homo sapiens
CGTEIGTDKLIF

SEQ ID NO: 33 - protein, 25 aa, Homo sapiens
QQPVQSPQAVILREGEDAVINCSSS

SEQ ID NO: 34 - protein, 17 aa, Homo sapiens
VHWYRQKHGEAPIFLMI

SEQ ID NO: 35 - protein, 33 aa, Homo sapiens
KGHDKISASFNEKKQQSSLYLTASQLSYSGTYF

SEQ ID NO: 36 - protein, 10 aa, Homo sapiens
GTGTRLQVFP

SEQ ID NO: 37 - DNA, 346 bp, Homo sapiens
  source 1..346: mol_type = other DNA
aatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagttacctcatcggggcggccgatgaaaaactgttttttggcagtggaacccagctctctgtcttgg

SEQ ID NO: 38 - protein, 115 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSYLIGAADEKLFFGSGTQLSVL

SEQ ID NO: 39 - protein, 5 aa, Homo sapiens
MNHNS

SEQ ID NO: 40 - protein, 6 aa, Homo sapiens
SASEGT

SEQ ID NO: 41 - protein, 16 aa, Homo sapiens
CASSYLIGAADEKLFF

SEQ ID NO: 42 - protein, 26 aa, Homo sapiens
NAGVTQTPKFQVLKTGQSMTLQCAQD

SEQ ID NO: 43 - protein, 17 aa, Homo sapiens
MYWYRQDPGMGLRLIYY

SEQ ID NO: 44 - protein, 36 aa, Homo sapiens
TDKGEVPNGYNVSRLNKREFSLRLESAAPSQTSVYF

SEQ ID NO: 45 - protein, 9 aa, Homo sapiens
GSGTQLSVL

SEQ ID NO: 46 - DNA, 355 bp, Homo sapiens
  source 1..355: mol_type = other DNA
gctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgagaaggagacaactgacagctgggggaaattgcagtttggagcagggacccaggttgtggtcaccccag

SEQ ID NO: 47 - protein, 118 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEKETTDSWGKLQFGAGTQVVVTP

SEQ ID NO: 48 - protein, 7 aa, Homo sapiens
TRDTTYY

SEQ ID NO: 49 - protein, 8 aa, Homo sapiens
RNSFDEQN

SEQ ID NO: 50 - protein, 17 aa, Homo sapiens
CALSEKETTDSWGKLQF

SEQ ID NO: 51 - protein, 26 aa, Homo sapiens
AQKVTQAQTEISVVEKEDVTLDCVYE

SEQ ID NO: 52 - protein, 17 aa, Homo sapiens
LFWYKQPPSGELVFLIR

SEQ ID NO: 53 - protein, 33 aa, Homo sapiens
EISGRYSWNFQKSTSSFNFTITASQVVDSAVYF

SEQ ID NO: 54 - protein, 10 aa, Homo sapiens
GAGTQVVVTP

SEQ ID NO: 55 - DNA, 358 bp, Homo sapiens
  source 1..358: mol_type = other DNA
gaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttatttctgtgccagcagccaagatgggggccgcagggggctgtggaccggggagctgttttttggagaaggctctaggctgaccgtactgg

SEQ ID NO: 56 - protein, 119 aa, Homo sapiens
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSQDGGRRGLWTGELFFGEGSRLTVL

SEQ ID NO: 57 - protein, 5 aa, Homo sapiens
SGHDN

SEQ ID NO: 58 - protein, 6 aa, Homo sapiens
FVKESK

SEQ ID NO: 59 - protein, 19 aa, Homo sapiens
CASSQDGGRRGLWTGELFF

SEQ ID NO: 60 - protein, 26 aa, Homo sapiens
EAGVTQFPSHSVIEKGQTVTLRCDPI

SEQ ID NO: 61 - protein, 17 aa, Homo sapiens
LYWYRRVMGKEIKFLLH

SEQ ID NO: 62 - protein, 37 aa, Homo sapiens
QDESGMPNNRFLAERTGGTYSTLKVQPAELEDSGVYF

SEQ ID NO: 63 - protein, 9 aa, Homo sapiens
GEGSRLTVL

SEQ ID NO: 64 - DNA, 355 bp, Homo sapiens
  source 1..355: mol_type = other DNA
gatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatccccacgggactagcggggtcagacaagagacccagtacttcgggccaggcacgcggctcctggtgctcg

SEQ ID NO: 65 - protein, 118 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFCATSDPHGTSGVRQETQYFGPGTRLLVL

SEQ ID NO: 66 - protein, 19 aa, Homo sapiens
CATSDPHGTSGVRQETQYF

SEQ ID NO: 67 - protein, 9 aa, Homo sapiens
GPGTRLLVL

SEQ ID NO: 68 - DNA, 337 bp, Homo sapiens
  source 1..337: mol_type = other DNA
gcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagactcagccatgtattactgtgctgccactaatgctggtggtactagctatggaaagctgacatttggacaagggaccatcttgactgtccatccaa

SEQ ID NO: 69 - protein, 112 aa, Homo sapiens
AQRVTQPEKLLSVFKGAPVELKCNYSYSGSPELFWYVQYSRQRLQLLLRHISRESIKGFTADLNKGETSFHLKKPFAQEEDSAMYYCAATNAGGTSYGKLTFGQGTILTVHP

SEQ ID NO: 70 - protein, 6 aa, Homo sapiens
YSGSPE

SEQ ID NO: 71 - protein, 4 aa, Homo sapiens
HISR

SEQ ID NO: 72 - protein, 16 aa, Homo sapiens
CAATNAGGTSYGKLTF

SEQ ID NO: 73 - protein, 26 aa, Homo sapiens
AQRVTQPEKLLSVFKGAPVELKCNYS

SEQ ID NO: 74 - protein, 17 aa, Homo sapiens
LFWYVQYSRQRLQLLLR

SEQ ID NO: 75 - protein, 33 aa, Homo sapiens
ESIKGFTADLNKGETSFHLKKPFAQEEDSAMYY

SEQ ID NO: 76 - protein, 10 aa, Homo sapiens
GQGTILTVHP

SEQ ID NO: 77 - DNA, 349 bp, Homo sapiens
  source 1..349: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctgatagcgggggaaccccctacgagcagtacttcgggccgggcaccaggctcacggtcacag

SEQ ID NO: 78 - protein, 116 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSADSGGTPYEQYFGPGTRLTVT

SEQ ID NO: 79 - protein, 6 aa, Homo sapiens
DFQATT

SEQ ID NO: 80 - protein, 7 aa, Homo sapiens
SNEGSKA

SEQ ID NO: 81 - protein, 14 aa, Homo sapiens
CSADSGGTPYEQYF

SEQ ID NO: 82 - protein, 26 aa, Homo sapiens
GAVVSQHPSWVICKSGTSVKIECRSL

SEQ ID NO: 83 - protein, 17 aa, Homo sapiens
MFWYRQFPKQSLMLMAT

SEQ ID NO: 84 - protein, 37 aa, Homo sapiens
TYEQGVEKDKFLINHASLTLSTLTVTSAHPEDSSFYI

SEQ ID NO: 85 - protein, 9 aa, Homo sapiens
GPGTRLTVT

SEQ ID NO: 86 - DNA, 331 bp, Homo sapiens
  source 1..331: mol_type = other DNA
gatgctaagaccacccagcccacctccatggattgcgctgaaggaagagctgcaaacctgccttgtaatcactctaccatcagtggaaatgagtatgtgtattggtatcgacagattcactcccaggggccacagtatatcattcatggtctaaaaaacaatgaaaccaatgaaatggcctctctgatcatcacagaagacagaaagtccagcaccttgatcctgccccacgctacgctgagagacactgctgtgtactattgcatcgtcagagcgaacaccggtaaccagttctattttgggacagggacaagtttgacggtcattccaa

SEQ ID NO: 87 - protein, 110 aa, Homo sapiens
DAKTTQPTSMDCAEGRAANLPCNHSTISGNEYVYWYRQIHSQGPQYIIHGLKNNETNEMASLIITEDRKSSTLILPHATLRDTAVYYCIVRANTGNQFYFGTGTSLTVIP

SEQ ID NO: 88 - protein, 7 aa, Homo sapiens
TISGNEY

SEQ ID NO: 89 - protein, 5 aa, Homo sapiens
GLKNN

SEQ ID NO: 90 - protein, 13 aa, Homo sapiens
CIVRANTGNQFYF

SEQ ID NO: 91 - protein, 25 aa, Homo sapiens
DAKTTQPTSMDCAEGRAANLPCNHS

SEQ ID NO: 92 - protein, 17 aa, Homo sapiens
VYWYRQIHSQGPQYIIH

SEQ ID NO: 93 - protein, 33 aa, Homo sapiens
ETNEMASLIITEDRKSSTLILPHATLRDTAVYY

SEQ ID NO: 94 - protein, 10 aa, Homo sapiens
GTGTSLTVIP

SEQ ID NO: 95 - DNA, 346 bp, Homo sapiens
  source 1..346: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgaagagggggacagatactatggctacaccttcggttcggggaccaggttaaccgttgtag

SEQ ID NO: 96 - protein, 115 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVEEGDRYYGYTFGSGTRLTVV

SEQ ID NO: 97 - protein, 5 aa, Homo sapiens
SQVTM

SEQ ID NO: 98 - protein, 7 aa, Homo sapiens
ANQGSEA

SEQ ID NO: 99 - protein, 14 aa, Homo sapiens
CSVEEGDRYYGYTF

SEQ ID NO: 100 - protein, 26 aa, Homo sapiens
SAVISQKPSRDICQRGTSLTIQCQVD

SEQ ID NO: 101 - protein, 17 aa, Homo sapiens
MFWYRQQPGQSLTLIAT

SEQ ID NO: 102 - protein, 37 aa, Homo sapiens
TYESGFVIDKFPISRPNLTFSTLTVSNMSPEDSSIYL

SEQ ID NO: 103 - protein, 9 aa, Homo sapiens
GSGTRLTVV

SEQ ID NO: 104 - DNA, 343 bp, Homo sapiens
  source 1..343: mol_type = other DNA
ggagattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgagctggtcggatggtggaggcttcaaaactatctttggagcaggaacaagactatttgttaaagcaa

SEQ ID NO: 105 - protein, 114 aa, Homo sapiens
GDSVTQMEGPVTLSEEAFLTINCTYTATGYPSLFWYVQYPGEGLQLLLKATKADDKGSNKGFEATYRKETTSFHLEKGSVQVSDSAVYFCALSWSDGGGFKTIFGAGTRLFVKA

SEQ ID NO: 106 - protein, 6 aa, Homo sapiens
ATGYPS

SEQ ID NO: 107 - protein, 7 aa, Homo sapiens
ATKADDK

SEQ ID NO: 108 - protein, 15 aa, Homo sapiens
CALSWSDGGGFKTIF

SEQ ID NO: 109 - protein, 26 aa, Homo sapiens
GDSVTQMEGPVTLSEEAFLTINCTYT

SEQ ID NO: 110 - protein, 17 aa, Homo sapiens
LFWYVQYPGEGLQLLLK

SEQ ID NO: 111 - protein, 33 aa, Homo sapiens
GSNKGFEATYRKETTSFHLEKGSVQVSDSAVYF

SEQ ID NO: 112 - protein, 10 aa, Homo sapiens
GAGTRLFVKA

SEQ ID NO: 113 - DNA, 352 bp, Homo sapiens
  source 1..352: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgccaggggggtggggtccccttcctacgagcagtacttcgggccgggcaccaggctcacggtcacag

SEQ ID NO: 114 - protein, 117 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARGVGSPSYEQYFGPGTRLTVT

SEQ ID NO: 115 - protein, 15 aa, Homo sapiens
CSARGVGSPSYEQYF

SEQ ID NO: 116 - DNA, 334 bp, Homo sapiens
  source 1..334: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgcagggaagccaaggaaatctcatctttggaaaaggcactaaactctctgttaaaccaa

SEQ ID NO: 117 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVQGSQGNLIFGKGTKLSVKP

SEQ ID NO: 118 - protein, 6 aa, Homo sapiens
DSAIYN

SEQ ID NO: 119 - protein, 7 aa, Homo sapiens
IQSSQRE

SEQ ID NO: 120 - protein, 12 aa, Homo sapiens
CAVQGSQGNLIF

SEQ ID NO: 121 - protein, 26 aa, Homo sapiens
KQEVTQIPAALSVPEGENLVLNCSFT

SEQ ID NO: 122 - protein, 17 aa, Homo sapiens
LQWFRQDPGKGLTSLLL

SEQ ID NO: 123 - protein, 33 aa, Homo sapiens
QTSGRLNASLDKSSGRSTLYIAASQPGDSATYL

SEQ ID NO: 124 - protein, 10 aa, Homo sapiens
GKGTKLSVKP

SEQ ID NO: 125 - DNA, 337 bp, Homo sapiens
  source 1..337: mol_type = other DNA
gagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagctcggataaggccccttcccagtacttcgggccaggcacgcggctcctggtgctcg

SEQ ID NO: 126 - protein, 112 aa, Homo sapiens
ETGVTQSPTHLIKTRGQQVTLRCSSQSGHNTVSWYQQALGQGPQFIFQYYREEENGRGNFPPRFSGLQFPNYSSELNVNALELDDSALYLCASSSDKAPSQYFGPGTRLLVL

SEQ ID NO: 127 - protein, 5 aa, Homo sapiens
SGHNT

SEQ ID NO: 128 - protein, 6 aa, Homo sapiens
YYREEE

SEQ ID NO: 129 - protein, 13 aa, Homo sapiens
CASSSDKAPSQYF

SEQ ID NO: 130 - protein, 26 aa, Homo sapiens
ETGVTQSPTHLIKTRGQQVTLRCSSQ

SEQ ID NO: 131 - protein, 17 aa, Homo sapiens
VSWYQQALGQGPQFIFQ

SEQ ID NO: 132 - protein, 36 aa, Homo sapiens
NGRGNFPPRFSGLQFPNYSSELNVNALELDDSALYL

SEQ ID NO: 133 - DNA, 334 bp, Homo sapiens
  source 1..334: mol_type = other DNA
aaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtggtgaacaatgcaggcaacatgctcacctttggagggggaacaaggttaatggtcaaacccc

SEQ ID NO: 134 - protein, 111 aa, Homo sapiens
KNQVEQSPQSLIILEGKNCTLQCNYTVSPFSNLRWYKQDTGRGPVSLTIMTFSENTKSNGRYTATLDADTKQSSLHITASQLSDSASYICVVNNAGNMLTFGGGTRLMVKP

SEQ ID NO: 135 - protein, 6 aa, Homo sapiens
VSPFSN

SEQ ID NO: 136 - protein, 7 aa, Homo sapiens
MTFSENT

SEQ ID NO: 137 - protein, 12 aa, Homo sapiens
CVVNNAGNMLTF

SEQ ID NO: 138 - protein, 26 aa, Homo sapiens
KNQVEQSPQSLIILEGKNCTLQCNYT

SEQ ID NO: 139 - protein, 17 aa, Homo sapiens
LRWYKQDTGRGPVSLTI

SEQ ID NO: 140 - protein, 33 aa, Homo sapiens
KSNGRYTATLDADTKQSSLHITASQLSDSASYI

SEQ ID NO: 141 - protein, 10 aa, Homo sapiens
GGGTRLMVKP

SEQ ID NO: 142 - DNA, 343 bp, Homo sapiens
  source 1..343: mol_type = other DNA
aatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcactagggacgggaaattcacccctccactttgggaacgggaccaggctcactgtgacag

SEQ ID NO: 143 - protein, 114 aa, Homo sapiens
NAGVMQNPRHLVRRRGQEARLRCSPMKGHSHVYWYRQLPEEGLKFMVYLQKENIIDESGMPKERFSAEFPKEGPSILRIQQVVRGDSAAYFCASSLGTGNSPLHFGNGTRLTVT

SEQ ID NO: 144 - protein, 5 aa, Homo sapiens
KGHSH

SEQ ID NO: 145 - protein, 6 aa, Homo sapiens
LQKENI

SEQ ID NO: 146 - protein, 14 aa, Homo sapiens
CASSLGTGNSPLHF

SEQ ID NO: 147 - protein, 26 aa, Homo sapiens
NAGVMQNPRHLVRRRGQEARLRCSPM

SEQ ID NO: 148 - protein, 17 aa, Homo sapiens
VYWYRQLPEEGLKFMVY

SEQ ID NO: 149 - protein, 37 aa, Homo sapiens
IDESGMPKERFSAEFPKEGPSILRIQQVVRGDSAAYF

SEQ ID NO: 150 - protein, 9 aa, Homo sapiens
GNGTRLTVT

SEQ ID NO: 151 - DNA, 337 bp, Homo sapiens
  source 1..337: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagagactatgataatgcaggcaacatgctcacctttggagggggaacaaggttaatggtcaaacccc

SEQ ID NO: 152 - protein, 112 aa, Homo sapiens
DAKTTQPNSMESNEEEPVHLPCNHSTISGTDYIHWYRQLPSQGPEYVIHGLTSNVNNRMASLAIAEDRKSSTLILHRATLRDAAVYYCILRDYDNAGNMLTFGGGTRLMVKP

SEQ ID NO: 153 - protein, 7 aa, Homo sapiens
TISGTDY

SEQ ID NO: 154 - protein, 5 aa, Homo sapiens
GLTSN

SEQ ID NO: 155 - protein, 15 aa, Homo sapiens
CILRDYDNAGNMLTF

SEQ ID NO: 156 - protein, 25 aa, Homo sapiens
DAKTTQPNSMESNEEEPVHLPCNHS

SEQ ID NO: 157 - protein, 17 aa, Homo sapiens
IHWYRQLPSQGPEYVIH

SEQ ID NO: 158 - protein, 33 aa, Homo sapiens
VNNRMASLAIAEDRKSSTLILHRATLRDAAVYY

SEQ ID NO: 159 - DNA, 340 bp, Homo sapiens
  source 1..340: mol_type = other DNA
gatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctacgttgtctccagatccaagacagagaatttccccctcactctggagtcagctacccgctcccagacatctgtgtatttctgcgccagcagccccggcgaaggggttgggggctacaccttcggttcggggaccaggttaaccgttgtag

SEQ ID NO: 160 - protein, 113 aa, Homo sapiens
DAGITQSPRYKITETGRQVTLMCHQTWSHSYMFWYRQDLGHGLRLIYYSAAADITDKGEVPDGYVVSRSKTENFPLTLESATRSQTSVYFCASSPGEGVGGYTFGSGTRLTVV

SEQ ID NO: 161 - protein, 5 aa, Homo sapiens
WSHSY

SEQ ID NO: 162 - protein, 6 aa, Homo sapiens
SAAADI

SEQ ID NO: 163 - protein, 14 aa, Homo sapiens
CASSPGEGVGGYTF

SEQ ID NO: 164 - protein, 26 aa, Homo sapiens
DAGITQSPRYKITETGRQVTLMCHQT

SEQ ID NO: 165 - protein, 17 aa, Homo sapiens
MFWYRQDLGHGLRLIYY

SEQ ID NO: 166 - protein, 36 aa, Homo sapiens
TDKGEVPDGYVVSRSKTENFPLTLESATRSQTSVYF

SEQ ID NO: 167 - DNA, 343 bp, Homo sapiens
  source 1..343: mol_type = other DNA
gaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagactcggccctgtatctctgtgccagcagccaaaccacgacagggggaggggactacaccttcggttcggggaccaggttaaccgttgtag

SEQ ID NO: 168 - protein, 114 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSQTTTGGGDYTFGSGTRLTVV

SEQ ID NO: 169 - protein, 5 aa, Homo sapiens
LGHNA

SEQ ID NO: 170 - protein, 6 aa, Homo sapiens
YNFKEQ

SEQ ID NO: 171 - protein, 15 aa, Homo sapiens
CASSQTTTGGGDYTF

SEQ ID NO: 172 - protein, 26 aa, Homo sapiens
ETGVTQTPRHLVMGMTNKKSLKCEQH

SEQ ID NO: 173 - protein, 17 aa, Homo sapiens
MYWYKQSAKKPLELMFV

SEQ ID NO: 174 - protein, 36 aa, Homo sapiens
TENNSVPSRFSPECPNSSHLFLHLHTLQPEDSALYL

SEQ ID NO: 175 - DNA, 334 bp, Homo sapiens
  source 1..334: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacgccgggggttaccagaaagttacctttggaattggaacaaagctccaagtcatcccaa

SEQ ID NO: 176 - protein, 111 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVNAGGYQKVTFGIGTKLQVIP

SEQ ID NO: 177 - protein, 13 aa, Homo sapiens
CAVNAGGYQKVTF

SEQ ID NO: 178 - protein, 10 aa, Homo sapiens
GIGTKLQVIP

SEQ ID NO: 179 - DNA, 340 bp, Homo sapiens
  source 1..340: mol_type = other DNA
gatgctggaatcacccagagcccaagacacaaggtcacagagacaggaacaccagtgactctgagatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcaagggagacgcgagcaatcagccccagcattttggtgatgggactcgactctccatcctag

SEQ ID NO: 180 - protein, 113 aa, Homo sapiens
DAGITQSPRHKVTETGTPVTLRCHQTENHRYMYWYRQDPGHGLRLIHYSYGVKDTDKGEVSDGYSVSRSKTEDFLLTLESATSSQTSVYFCAIKGDASNQPQHFGDGTRLSIL

SEQ ID NO: 181 - protein, 5 aa, Homo sapiens
ENHRY

SEQ ID NO: 182 - protein, 6 aa, Homo sapiens
SYGVKD

SEQ ID NO: 183 - protein, 14 aa, Homo sapiens
CAIKGDASNQPQHF

SEQ ID NO: 184 - protein, 26 aa, Homo sapiens
DAGITQSPRHKVTETGTPVTLRCHQT

SEQ ID NO: 185 - protein, 17 aa, Homo sapiens
MYWYRQDPGHGLRLIHY

SEQ ID NO: 186 - protein, 36 aa, Homo sapiens
TDKGEVSDGYSVSRSKTEDFLLTLESATSSQTSVYF

SEQ ID NO: 187 - DNA, 346 bp, Homo sapiens
  source 1..346: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgagctctgacagaggctcaaccctggggaggctatactttggaagaggaactcagttgactgtctggcctg

SEQ ID NO: 188 - protein, 115 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSSDRGSTLGRLYFGRGTQLTVWP

SEQ ID NO: 189 - protein, 16 aa, Homo sapiens
CAVSSDRGSTLGRLYF

SEQ ID NO: 190 - protein, 10 aa, Homo sapiens
GRGTQLTVWP

SEQ ID NO: 191 - DNA, 358 bp, Homo sapiens
  source 1..358: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagatatgggacagaaaattagcaatcagccccagcattttggtgatgggactcgactctccatcctag

SEQ ID NO: 192 - protein, 119 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDMGQKISNQPQHFGDGTRLSIL

SEQ ID NO: 193 - protein, 17 aa, Homo sapiens
CSARDMGQKISNQPQHF

SEQ ID NO: 194 - DNA, 331 bp, Homo sapiens
  source 1..331: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtggatgcaggcaacatgctcacctttggagggggaacaaggttaatggtcaaacccc

SEQ ID NO: 195 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVDAGNMLTFGGGTRLMVKP

SEQ ID NO: 196 - protein, 11 aa, Homo sapiens
CAVDAGNMLTF

SEQ ID NO: 197 - DNA, 343 bp, Homo sapiens
  source 1..343: mol_type = other DNA
gatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcggccctgggtatagttataattcacccctccactttgggaacgggaccaggctcactgtgacag

SEQ ID NO: 198 - protein, 114 aa, Homo sapiens
DVKVTQSSRYLVKRTGEKVFLECVQDMDHENMFWYRQDPGLGLRLIYFSYDVKMKEKGDIPEGYSVSREKKERFSLILESASTNQTSMYLCASGPGYSYNSPLHFGNGTRLTVT

SEQ ID NO: 199 - protein, 5 aa, Homo sapiens
MDHEN

SEQ ID NO: 200 - protein, 6 aa, Homo sapiens
SYDVKM

SEQ ID NO: 201 - protein, 15 aa, Homo sapiens
CASGPGYSYNSPLHF

SEQ ID NO: 202 - protein, 26 aa, Homo sapiens
DVKVTQSSRYLVKRTGEKVFLECVQD

SEQ ID NO: 203 - protein, 17 aa, Homo sapiens
MFWYRQDPGLGLRLIYF

SEQ ID NO: 204 - protein, 36 aa, Homo sapiens
KEKGDIPEGYSVSREKKERFSLILESASTNQTSMYL

SEQ ID NO: 205 - DNA, 331 bp, Homo sapiens
  source 1..331: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgtcactggttctgcaaggcaactgacctttggatctgggacacaattgactgttttacctg

SEQ ID NO: 206 - protein, 110 aa, Homo sapiens
GENVEQHPSTLSVQEGDSAVIKCTYSDSASNYFPWYKQELGKRPQLIIDIRSNVGEKKDQRIAVTLNKTAKHFSLHITETQPEDSAVYFCVTGSARQLTFGSGTQLTVLP

SEQ ID NO: 207 - protein, 6 aa, Homo sapiens
DSASNY

SEQ ID NO: 208 - protein, 7 aa, Homo sapiens
IRSNVGE

SEQ ID NO: 209 - protein, 11 aa, Homo sapiens
CVTGSARQLTF

SEQ ID NO: 210 - protein, 26 aa, Homo sapiens
GENVEQHPSTLSVQEGDSAVIKCTYS

SEQ ID NO: 211 - protein, 17 aa, Homo sapiens
FPWYKQELGKRPQLIID

SEQ ID NO: 212 - protein, 33 aa, Homo sapiens
KKDQRIAVTLNKTAKHFSLHITETQPEDSAVYF

SEQ ID NO: 213 - protein, 10 aa, Homo sapiens
GSGTQLTVLP

SEQ ID NO: 214 - DNA, 346 bp, Homo sapiens
  source 1..346: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagcccagggtggcaaagcagatcctatggctacaccttcggttcggggaccaggttaaccgttgtag

SEQ ID NO: 215 - protein, 115 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPGWQSRSYGYTFGSGTRLTVV

SEQ ID NO: 216 - protein, 5 aa, Homo sapiens
LNHDA

SEQ ID NO: 217 - protein, 6 aa, Homo sapiens
SQIVND

SEQ ID NO: 218 - protein, 16 aa, Homo sapiens
CASSPGWQSRSYGYTF

SEQ ID NO: 219 - protein, 26 aa, Homo sapiens
DGGITQSPKYLFRKEGQNVTLSCEQN

SEQ ID NO: 220 - protein, 17 aa, Homo sapiens
MYWYRQDPGQGLRLIYY

SEQ ID NO: 221 - protein, 36 aa, Homo sapiens
FQKGDIAEGYSVSREKKESFPLTVTSAQKNPTAFYL

SEQ ID NO: 222 - DNA, 322 bp, Homo sapiens
  source 1..322: mol_type = other DNA
cggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaacaatgccagactcatgtttggagatggaactcagctggtggtgaagccca

SEQ ID NO: 223 - protein, 107 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVNNARLMFGDGTQLVVKP

SEQ ID NO: 224 - protein, 6 aa, Homo sapiens
NSASQS

SEQ ID NO: 225 - protein, 5 aa, Homo sapiens
VYSSG

SEQ ID NO: 226 - protein, 10 aa, Homo sapiens
CVVNNARLMF

SEQ ID NO: 227 - protein, 26 aa, Homo sapiens
RKEVEQDPGPFNVPEGATVAFNCTYS

SEQ ID NO: 228 - protein, 17 aa, Homo sapiens
FFWYRQDCRKEPKLLMS

SEQ ID NO: 229 - protein, 33 aa, Homo sapiens
NEDGRFTAQLNRASQYISLLIRDSKLSDSATYL

SEQ ID NO: 230 - protein, 10 aa, Homo sapiens
GDGTQLVVKP

SEQ ID NO: 231 - DNA, 349 bp, Homo sapiens
  source 1..349: mol_type = other DNA
gatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatttgtcctcccgggacagccaagagacccagtacttcgggccaggcacgcggctcctggtgctcg

SEQ ID NO: 232 - protein, 116 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFCATSDLSSRDSQETQYFGPGTRLLVL

SEQ ID NO: 233 - protein, 17 aa, Homo sapiens
CATSDLSSRDSQETQYF

SEQ ID NO: 234 - DNA, 319 bp, Homo sapiens
  source 1..319: mol_type = other DNA
aaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctcccggggctggcaacaaccgtaagctgatttggggattgggaacaagcctggcagtaaatccga

SEQ ID NO: 235 - protein, 106 aa, Homo sapiens
KDQVFQPSTVASSEGAVVEIFCNHSVSNAYNFFWYLHFPGCAPRLLVKGSKPSQQGRYNMTYERFSSSLLILQVREADAAVYYCAPGAGNNRKLIWGLGTSLAVNP

SEQ ID NO: 236 - protein, 6 aa, Homo sapiens
VSNAYN

SEQ ID NO: 237 - protein, 4 aa, Homo sapiens
GSKP

SEQ ID NO: 238 - protein, 13 aa, Homo sapiens
CAPGAGNNRKLIW

SEQ ID NO: 239 - protein, 25 aa, Homo sapiens
KDQVFQPSTVASSEGAVVEIFCNHS

SEQ ID NO: 240 - protein, 17 aa, Homo sapiens
FFWYLHFPGCAPRLLVK

SEQ ID NO: 241 - protein, 31 aa, Homo sapiens
SQQGRYNMTYERFSSSLLILQVREADAAVYY

SEQ ID NO: 242 - protein, 10 aa, Homo sapiens
GLGTSLAVNP

SEQ ID NO: 243 - DNA, 337 bp, Homo sapiens
  source 1..337: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtcggacaggtaacactgaagctttctttggacaaggcaccagactcacagttgtag

SEQ ID NO: 244 - protein, 112 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSRTGNTEAFFGQGTRLTVV

SEQ ID NO: 245 - protein, 13 aa, Homo sapiens
CASSRTGNTEAFF

SEQ ID NO: 246 - protein, 9 aa, Homo sapiens
GQGTRLTVV

SEQ ID NO: 247 - DNA, 343 bp, Homo sapiens
  source 1..343: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcctccacgggaatatggaaacaaactggtctttggcgcaggaaccattctgagagtcaagtcct

SEQ ID NO: 248 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPPREYGNKLVFGAGTILRVKS

SEQ ID NO: 249 - protein, 15 aa, Homo sapiens
CAVRPPREYGNKLVF

SEQ ID NO: 250 - DNA, 337 bp, Homo sapiens
  source 1..337: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacggggaggggaacgagcagtacttcgggccgggcaccaggctcacggtcacag

SEQ ID NO: 251 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYGEGNEQYFGPGTRLTVT

SEQ ID NO: 252 - protein, 5 aa, Homo sapiens
MNHEY

SEQ ID NO: 253 - protein, 6 aa, Homo sapiens
SVGAGI

SEQ ID NO: 254 - protein, 13 aa, Homo sapiens
CASSYGEGNEQYF

SEQ ID NO: 255 - protein, 17 aa, Homo sapiens
MSWYRQDPGMGLRLIHY

SEQ ID NO: 256 - protein, 36 aa, Homo sapiens
TDQGEVPNGYNVSRSTTEDFPLRLLSAAPSQTSVYF

SEQ ID NO: 257 - DNA, 343 bp, Homo sapiens
  source 1..343: mol_type = other DNA
aaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggggagagggcacgggcaggagagcacttacttttgggagtggaacaagactccaagtgcaaccaa

SEQ ID NO: 258 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRGEGTGRRALTFGSGTRLQVQP

SEQ ID NO: 259 - protein, 15 aa, Homo sapiens
CAVRGEGTGRRALTF

SEQ ID NO: 260 - protein, 10 aa, Homo sapiens
GSGTRLQVQP

SEQ ID NO: 261 - DNA, 346 bp, Homo sapiens
  source 1..346: mol_type = other DNA
ggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctctgtgccagcagcaatagcgggacatggggagagacccagtacttcgggccaggcacgcggctcctggtgctcg

SEQ ID NO: 262 - protein, 115 aa, Homo sapiens
GAGVSQTPSNKVTEKGKYVELRCDPISGHTALYWYRQSLGQGPEFLIYFQGTGAADDSGLPNDRFFAVRPEGSVSTLKIQRTERGDSAVYLCASSNSGTWGETQYFGPGTRLLVL

SEQ ID NO: 263 - protein, 5 aa, Homo sapiens
SGHTA

SEQ ID NO: 264 - protein, 6 aa, Homo sapiens
FQGTGA

SEQ ID NO: 265 - protein, 15 aa, Homo sapiens
CASSNSGTWGETQYF

SEQ ID NO: 266 - protein, 26 aa, Homo sapiens
GAGVSQTPSNKVTEKGKYVELRCDPI

SEQ ID NO: 267 - protein, 17 aa, Homo sapiens
LYWYRQSLGQGPEFLIY

SEQ ID NO: 268 - protein, 37 aa, Homo sapiens
ADDSGLPNDRFFAVRPEGSVSTLKIQRTERGDSAVYL

SEQ ID NO: 269 - DNA, 337 bp, Homo sapiens
  source 1..337: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtcagagataactatggtcagaattttgtctttggtcccggaaccagattgtccgtgctgccct

SEQ ID NO: 270 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRDNYGQNFVFGPGTRLSVLP

SEQ ID NO: 271 - protein, 13 aa, Homo sapiens
CAVRDNYGQNFVF

SEQ ID NO: 272 - protein, 10 aa, Homo sapiens
GPGTRLSVLP

SEQ ID NO: 273 - DNA, 346 bp, Homo sapiens
  source 1..346: mol_type = other DNA
ggtgctggagtctcccagtctcccaggtacaaagtcacaaagaggggacaggatgtagctctcaggtgtgatccaatttcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgctgtgccagcagcttagggaccagcggggactacgagcagtacttcgggccgggcaccaggctcacggtcacag

SEQ ID NO: 274 - protein, 115 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSLGTSGDYEQYFGPGTRLTVT

SEQ ID NO: 275 - protein, 5 aa, Homo sapiens
SGHVS

SEQ ID NO: 276 - protein, 6 aa, Homo sapiens
FNYEAQ

SEQ ID NO: 277 - protein, 15 aa, Homo sapiens
CASSLGTSGDYEQYF

SEQ ID NO: 278 - protein, 26 aa, Homo sapiens
GAGVSQSPRYKVTKRGQDVALRCDPI

SEQ ID NO: 279 - protein, 17 aa, Homo sapiens
LYWYRQALGQGPEFLTY

SEQ ID NO: 280 - protein, 37 aa, Homo sapiens
QDKSGLPNDRFSAERPEGSISTLTIQRTEQRDSAMYR

SEQ ID NO: 281 - DNA, 349 bp, Homo sapiens
  source 1..349: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagcatagcggggggacaagagacccagtacttcgggccaggcacgcggctcctggtgctcg

SEQ ID NO: 282 - protein, 116 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSASIAGGQETQYFGPGTRLLVL

SEQ ID NO: 283 - protein, 14 aa, Homo sapiens
CSASIAGGQETQYF

SEQ ID NO: 284 - DNA, 355 bp, Homo sapiens
  source 1..355: mol_type = other DNA
gctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagcgcgtcgtttggctttggaaatgagaaattaacctttgggactggaacaagactcaccatcataccca

SEQ ID NO: 285 - protein, 118 aa, Homo sapiens
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRSASFGFGNEKLTFGTGTRLTIIP

SEQ ID NO: 286 - protein, 7 aa, Homo sapiens
TSESDYY

SEQ ID NO: 287 - protein, 8 aa, Homo sapiens
QEAYKQQN

SEQ ID NO: 288 - protein, 17 aa, Homo sapiens
CAYRSASFGFGNEKLTF

SEQ ID NO: 289 - protein, 26 aa, Homo sapiens
AQTVTQSQPEMSVQEAETVTLSCTYD

SEQ ID NO: 290 - protein, 17 aa, Homo sapiens
LFWYKQPPSRQMILVIR

SEQ ID NO: 291 - protein, 33 aa, Homo sapiens
ATENRFSVNFQKAAKSFSLKISDSQLGDAAMYF

SEQ ID NO: 292 - protein, 10 aa, Homo sapiens
GTGTRLTIIP

SEQ ID NO: 293 - DNA, 343 bp, Homo sapiens
  source 1..343: mol_type = other DNA
actgctgggatcacccaggcaccaacatctcagatcctggcagcaggacggagcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttctgtgccagcatcccgaacaggttctctaactatggctacaccttcggttcggggaccaggttaaccgttgtag

SEQ ID NO: 294 - protein, 114 aa, Homo sapiens
TAGITQAPTSQILAAGRSMTLRCTQDMRHNAMYWYRQDLGLGLRLIHYSNTAGTTGKGEVPDGYSVSRANTDDFPLTLASAVPSQTSVYFCASIPNRFSNYGYTFGSGTRLTVV

SEQ ID NO: 295 - protein, 5 aa, Homo sapiens
MRHNA

SEQ ID NO: 296 - protein, 6 aa, Homo sapiens
SNTAGT

SEQ ID NO: 297 - protein, 15 aa, Homo sapiens
CASIPNRFSNYGYTF

SEQ ID NO: 298 - protein, 26 aa, Homo sapiens
TAGITQAPTSQILAAGRSMTLRCTQD

SEQ ID NO: 299 - protein, 17 aa, Homo sapiens
MYWYRQDLGLGLRLIHY

SEQ ID NO: 300 - protein, 36 aa, Homo sapiens
TGKGEVPDGYSVSRANTDDFPLTLASAVPSQTSVYF

SEQ ID NO: 301 - DNA, 343 bp, Homo sapiens
  source 1..343: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagggagaattctggttctgcaaggcaactgacctttggatctgggacacaattgactgttttacctg

SEQ ID NO: 302 - protein, 114 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAARENSGSARQLTFGSGTQLTVLP

SEQ ID NO: 303 - protein, 6 aa, Homo sapiens
NSMFDY

SEQ ID NO: 304 - protein, 7 aa, Homo sapiens
ISSIKDK

SEQ ID NO: 305 - protein, 15 aa, Homo sapiens
CAARENSGSARQLTF

SEQ ID NO: 306 - protein, 26 aa, Homo sapiens
DQQVKQNSPSLSVQEGRISILNCDYT

SEQ ID NO: 307 - protein, 17 aa, Homo sapiens
FLWYKKYPAEGPTFLIS

SEQ ID NO: 308 - protein, 33 aa, Homo sapiens
NEDGRFTVFLNKSAKHLSLHIVPSQPGDSAVYF

SEQ ID NO: 309 - DNA, 331 bp, Homo sapiens
  source 1..331: mol_type = other DNA
gatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgacaacaaagagcagtacttcgggccgggcaccaggctcacggtcacag

SEQ ID NO: 310 - protein, 110 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFCATSDNKEQYFGPGTRLTVT

SEQ ID NO: 311 - protein, 11 aa, Homo sapiens
CATSDNKEQYF

SEQ ID NO: 312 - DNA, 328 bp, Homo sapiens
  source 1..328: mol_type = other DNA
ggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagaggaccgatacagcaccctcacctttgggaaggggactatgcttctagtctctccag

SEQ ID NO: 313 - protein, 109 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAEDRYSTLTFGKGTMLLVSP

SEQ ID NO: 314 - protein, 6 aa, Homo sapiens
DSSSTY

SEQ ID NO: 315 - protein, 7 aa, Homo sapiens
IFSNMDM

SEQ ID NO: 316 - protein, 11 aa, Homo sapiens
CAEDRYSTLTF

SEQ ID NO: 317 - protein, 25 aa, Homo sapiens
GEDVEQSLFLSVREGDSSVINCTYT

SEQ ID NO: 318 - protein, 17 aa, Homo sapiens
LYWYKQEPGAGLQLLTY

SEQ ID NO: 319 - protein, 33 aa, Homo sapiens
KQDQRLTVLLNKKDKHLSLRIADTQTGDSAIYF

SEQ ID NO: 320 - protein, 10 aa, Homo sapiens
GKGTMLLVSP

SEQ ID NO: 321 - DNA, 328 bp, Homo sapiens
  source 1..328: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtgaggcagatacgcagtattttggcccaggcacccggctgacagtgctcg

SEQ ID NO: 322 - protein, 109 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASEADTQYFGPGTRLTVL

SEQ ID NO: 323 - protein, 10 aa, Homo sapiens
CASEADTQYF

SEQ ID NO: 324 - DNA, 352 bp, Homo sapiens
  source 1..352: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacaggggaagggataaatctgtgaacaccggtaaccagttctattttgggacagggacaagtttgacggtcattccaa

SEQ ID NO: 325 - protein, 117 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVNRGRDKSVNTGNQFYFGTGTSLTVIP

SEQ ID NO: 326 - protein, 19 aa, Homo sapiens
CAVNRGRDKSVNTGNQFYF

SEQ ID NO: 327 - DNA, 328 bp, Homo sapiens; identical to SEQ ID NO: 321
  source 1..328: mol_type = other DNA

SEQ ID NO: 328 - protein, 109 aa, Homo sapiens; identical to SEQ ID NO: 322

SEQ ID NO: 329 - DNA, 334 bp, Homo sapiens
  source 1..334: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcaggggacaggaggagggctacaccttcggttcggggaccaggttaaccgttgtag

SEQ ID NO: 330 - protein, 111 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASRGQEEGYTFGSGTRLTVV

SEQ ID NO: 331 - protein, 5 aa, Homo sapiens
SGHDT

SEQ ID NO: 332 - protein, 6 aa, Homo sapiens
YYEEEE

SEQ ID NO: 333 - protein, 12 aa, Homo sapiens
CASRGQEEGYTF

SEQ ID NO: 334 - protein, 26 aa, Homo sapiens
DAGVTQSPTHLIKTRGQQVTLRCSPK

SEQ ID NO: 335 - protein, 36 aa, Homo sapiens
RQRGNFPDRFSGHQFPNYSSELNVNALLLGDSALYL

SEQ ID NO: 336 - DNA, 343 bp, Homo sapiens
  source 1..343: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccccggccagggcggatctgaaaagctggtctttggaaagggaacgaaactgacagtaaacccat

SEQ ID NO: 337 - protein, 114 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAATPGQGGSEKLVFGKGTKLTVNP

SEQ ID NO: 338 - protein, 15 aa, Homo sapiens
CAATPGQGGSEKLVF

SEQ ID NO: 339 - protein, 10 aa, Homo sapiens
GKGTKLTVNP

SEQ ID NO: 340 - DNA, 343 bp, Homo sapiens
  source 1..343: mol_type = other DNA
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaagtggcgggggggagcgatgagcagttcttcgggccagggacacggctcaccgtgctag

SEQ ID NO: 341 - protein, 114 aa, Homo sapiens
DTAVSQTPKYLVTQMGNDKSIKCEQNLGHDTMYWYKQDSKKFLKIMFSYNNKELIINETVPNRFSPKSPDKAHLNLHINSLELGDSAVYFCASSQVAGGSDEQFFGPGTRLTVL

SEQ ID NO: 342 - protein, 5 aa, Homo sapiens
LGHDT

SEQ ID NO: 343 - protein, 6 aa, Homo sapiens
YNNKEL

SEQ ID NO: 344 - protein, 15 aa, Homo sapiens
CASSQVAGGSDEQFF

SEQ ID NO: 345 - protein, 26 aa, Homo sapiens
DTAVSQTPKYLVTQMGNDKSIKCEQN

SEQ ID NO: 346 - protein, 17 aa, Homo sapiens
MYWYKQDSKKFLKIMFS

SEQ ID NO: 347 - protein, 36 aa, Homo sapiens
IINETVPNRFSPKSPDKAHLNLHINSLELGDSAVYF

SEQ ID NO: 348 - DNA, 331 bp, Homo sapiens
  source 1..331: mol_type = other DNA
ggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcgctgaacatgcaggtagcaactatcagttaatctggggcgctgggaccaagctaattataaagccag

SEQ ID NO: 349 - protein, 110 aa, Homo sapiens
GQSLEQPSEVTAVEGAIVQINCTYQTSGFYGLSWYQQHDGGAPTFLSYNALDGLEETGRFSSFLSRSDSYGYLLLQELQMKDSASYFCAEHAGSNYQLIWGAGTKLIIKP

SEQ ID NO: 350 - protein, 6 aa, Homo sapiens
TSGFYG

SEQ ID NO: 351 - protein, 6 aa, Homo sapiens
NALDGL

SEQ ID NO: 352 - protein, 13 aa, Homo sapiens
CAEHAGSNYQLIW

SEQ ID NO: 353 - protein, 25 aa, Homo sapiens
GQSLEQPSEVTAVEGAIVQINCTYQ

SEQ ID NO: 354 - protein, 17 aa, Homo sapiens
LSWYQQHDGGAPTFLSY

SEQ ID NO: 355 - protein, 33 aa, Homo sapiens
EETGRFSSFLSRSDSYGYLLLQELQMKDSASYF

SEQ ID NO: 356 - protein, 10 aa, Homo sapiens
GAGTKLIIKP

SEQ ID NO: 357 - DNA, 352 bp, Homo sapiens
  source 1..352: mol_type = other DNA
aatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcaccgaccgggacaggggataacaccggggagctgttttttggagaaggctctaggctgaccgtactgg

SEQ ID NO: 358 - protein, 117 aa, Homo sapiens
NAGVMQNPRHLVRRRGQEARLRCSPMKGHSHVYWYRQLPEEGLKFMVYLQKENIIDESGMPKERFSAEFPKEGPSILRIQQVVRGDSAAYFCASSPTGTGDNTGELFFGEGSRLTVL

SEQ ID NO: 359 - protein, 17 aa, Homo sapiens
CASSPTGTGDNTGELFF

SEQ ID NO: 360 - DNA, 346 bp, Homo sapiens
  source 1..346: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgagggcccgggaggaggtgctgacggactcacctttggcaaagggactcatctaatcatccagccct

SEQ ID NO: 361 - protein, 115 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASEGPGGGADGLTFGKGTHLIIQP

SEQ ID NO: 362 - protein, 16 aa, Homo sapiens
CAASEGPGGGADGLTF

SEQ ID NO: 363 - protein, 10 aa, Homo sapiens
GKGTHLIIQP

SEQ ID NO: 364 - DNA, 340 bp, Homo sapiens
  source 1..340: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttttcgggacagggaatcgaagctttctttggacaaggcaccagactcacagttgtag

SEQ ID NO: 365 - protein, 113 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFSGQGIEAFFGQGTRLTVV

SEQ ID NO: 366 - protein, 6 aa, Homo sapiens
SMNVEV

SEQ ID NO: 367 - protein, 14 aa, Homo sapiens
CASSFSGQGIEAFF

SEQ ID NO: 368 - protein, 26 aa, Homo sapiens
EAQVTQNPRYLITVTGKKLTVTCSQN

SEQ ID NO: 369 - protein, 17 aa, Homo sapiens
MSWYRQDPGLGLRQIYY

SEQ ID NO: 370 - protein, 36 aa, Homo sapiens
TDKGDVPEGYKVSRKEKRNFPLILESPSPNQTSLYF

SEQ ID NO: 371 - DNA, 340 bp, Homo sapiens
  source 1..340: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcagttaactctggttctgcaaggcaactgacctttggatctgggacacaattgactgttttacctg

SEQ ID NO: 372 - protein, 113 aa, Homo sapiens
GENVEQHPSTLSVQEGDSAVIKCTYSDSASNYFPWYKQELGKGPQLIIDIRSNVGEKKDQRIAVTLNKTAKHFSLHITETQPEDSAVYFCAAVNSGSARQLTFGSGTQLTVLP

SEQ ID NO: 373 - protein, 14 aa, Homo sapiens
CAAVNSGSARQLTF

SEQ ID NO: 374 - protein, 17 aa, Homo sapiens
FPWYKQELGKGPQLIID

SEQ ID NO: 375 - DNA, 343 bp, Homo sapiens
  source 1..343: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtaccctgactagcggagactacaatgagcagttcttcgggccagggacacggctcaccgtgctag

SEQ ID NO: 376 - protein, 114 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASTLTSGDYNEQFFGPGTRLTVL

SEQ ID NO: 377 - protein, 15 aa, Homo sapiens
CASTLTSGDYNEQFF

SEQ ID NO: 378 - DNA, 331 bp, Homo sapiens
  source 1..331: mol_type = other DNA
cggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtggatttttataacaccgacaagctcatctttgggactgggaccagattacaagtctttccaa

SEQ ID NO: 379 - protein, 110 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVDFYNTDKLIFGTGTRLQVFP

SEQ ID NO: 380 - protein, 13 aa, Homo sapiens
CVVDFYNTDKLIF

SEQ ID NO: 381 - DNA, 346 bp, Homo sapiens
  source 1..346: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcctcagtaagggggcgggaaccggggagctgttttttggagaaggctctaggctgaccgtactgg

SEQ ID NO: 382 - protein, 115 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSLSKGAGTGELFFGEGSRLTVL

SEQ ID NO: 383 - protein, 5 aa, Homo sapiens
SGDLS

SEQ ID NO: 384 - protein, 6 aa, Homo sapiens
YYNGEE

SEQ ID NO: 385 - protein, 16 aa, Homo sapiens
CASSLSKGAGTGELFF

SEQ ID NO: 386 - protein, 26 aa, Homo sapiens
DSGVTQTPKHLITATGQRVTLRCSPR

SEQ ID NO: 387 - protein, 17 aa, Homo sapiens
VYWYQQSLDQGLQFLIQ

SEQ ID NO: 388 - protein, 36 aa, Homo sapiens
RAKGNILERFSAQQFPDLHSELNLSSLELGDSALYF

SEQ ID NO: 389 - DNA, 331 bp, Homo sapiens
  source 1..331: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaacagatggccagaagctgctctttgcaagggggaccatgttaaaggtggatctta

SEQ ID NO: 390 - protein, 110 aa, Homo sapiens
GENVEQHPSTLSVQEGDSAVIKCTYSDSASNYFPWYKQELGKRPQLIIDIRSNVGEKKDQRIAVTLNKTAKHFSLHITETQPEDSAVYFCAATDGQKLLFARGTMLKVDL

SEQ ID NO: 391 - protein, 11 aa, Homo sapiens
CAATDGQKLLF

SEQ ID NO: 392 - protein, 10 aa, Homo sapiens
ARGTMLKVDL

SEQ ID NO: 393 - DNA, 331 bp, Homo sapiens
  source 1..331: mol_type = other DNA
tctcagactattcatcaatggccagcgaccctggtgcagcctgtgggcagcccgctctctctggagtgcactgtggagggaacatcaaaccccaacctatactggtaccgacaggctgcaggcaggggcctccagctgctcttctactccgttggtattggccagatcagctctgaggtgccccagaatctctcagcctccagaccccaggaccggcagttcatcctgagttctaagaagctccttctcagtgactctggcttctatctctgtgcctgggagggggtaaggggctacaccttcggttcggggaccaggttaaccgttgtag

SEQ ID NO: 394 - protein, 110 aa, Homo sapiens
SQTIHQWPATLVQPVGSPLSLECTVEGTSNPNLYWYRQAAGRGLQLLFYSVGIGQISSEVPQNLSASRPQDRQFILSSKKLLLSDSGFYLCAWEGVRGYTFGSGTRLTVV

SEQ ID NO: 395 - protein, 6 aa, Homo sapiens
GTSNPN

SEQ ID NO: 396 - protein, 5 aa, Homo sapiens
SVGIG

SEQ ID NO: 397 - protein, 11 aa, Homo sapiens
CAWEGVRGYTF

SEQ ID NO: 398 - protein, 26 aa, Homo sapiens
SQTIHQWPATLVQPVGSPLSLECTVE

SEQ ID NO: 399 - protein, 17 aa, Homo sapiens
LYWYRQAAGRGLQLLFY

SEQ ID NO: 400 - protein, 36 aa, Homo sapiens
QISSEVPQNLSASRPQDRQFILSSKKLLLSDSGFYL

SEQ ID NO: 401 - DNA, 349 bp, Homo sapiens
  source 1..349: mol_type = other DNA
gcccagaagataactcaaacccaaccaggaatgttcgtgcaggaaaaggaggctgtgactctggactgcacatatgacaccagtgatcaaagttatggtctcttctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgacctcttcttataaccagggaggaaagcttatcttcggacagggaacggagttatctgtgaaaccca

SEQ ID NO: 402 - protein, 116 aa, Homo sapiens
AQKITQTQPGMFVQEKEAVTLDCTYDTSDQSYGLFWYKQPSSGEMIFLIYQGSYDEQNATEGRYSLNFQKARKSANLVISASQLGDSAMYFCAMTSSYNQGGKLIFGQGTELSVKP

SEQ ID NO: 403 - protein, 7 aa, Homo sapiens
TSDQSYG

SEQ ID NO: 404 - protein, 8 aa, Homo sapiens
QGSYDEQN

SEQ ID NO: 405 - protein, 15 aa, Homo sapiens
CAMTSSYNQGGKLIF

SEQ ID NO: 406 - protein, 26 aa, Homo sapiens
AQKITQTQPGMFVQEKEAVTLDCTYD

SEQ ID NO: 407 - protein, 17 aa, Homo sapiens
LFWYKQPSSGEMIFLIY

SEQ ID NO: 408 - protein, 33 aa, Homo sapiens
ATEGRYSLNFQKARKSANLVISASQLGDSAMYF

SEQ ID NO: 409 - protein, 10 aa, Homo sapiens
GQGTELSVKP

SEQ ID NO: 410 - DNA, 343 bp, Homo sapiens
  source 1..343: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagaggggacagtccgagctttctttggacaaggcaccagactcacagttgtag

SEQ ID NO: 411 - protein, 114 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSAREGTVRAFFGQGTRLTVV

SEQ ID NO: 412 - protein, 12 aa, Homo sapiens
CSAREGTVRAFF

SEQ ID NO: 413 - DNA, 337 bp, Homo sapiens
  source 1..337: mol_type = other DNA
gaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcgtctttcttctggttctgcaaggcaactgacctttggatctgggacacaattgactgttttacctg

SEQ ID NO: 414 - protein, 112 aa, Homo sapiens
EDQVTQSPEALRLQEGESSSLNCSYTVSGLRGLFWYRQDPGKGPEFLFTLYSAGEEKEKERLKATLTKKESFLHITAPKPEDSATYLCAVRLSSGSARQLTFGSGTQLTVLP

SEQ ID NO: 415 - protein, 6 aa, Homo sapiens
VSGLRG

SEQ ID NO: 416 - protein, 7 aa, Homo sapiens
LYSAGEE

SEQ ID NO: 417 - protein, 15 aa, Homo sapiens
CAVRLSSGSARQLTF

SEQ ID NO: 418 - protein, 26 aa, Homo sapiens
EDQVTQSPEALRLQEGESSSLNCSYT

SEQ ID NO: 419 - protein, 17 aa, Homo sapiens
LFWYRQDPGKGPEFLFT

SEQ ID NO: 420 - protein, 31 aa, Homo sapiens
KEKERLKATLTKKESFLHITAPKPEDSATYL

SEQ ID NO: 421 - DNA, 331 bp, Homo sapiens
  source 1..331: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcactattataatggagaagagagagcaaaaggaaacattcttgaacgattctccgcacaacagttccctgacttgcactctgaactaaacctgagctctctggagctgggggactcagctttgtatttctgtgccagcagcgactctcatacgcagtattttggcccaggcacccggctgacagtgctcg

SEQ ID NO: 422 - protein, 110 aa, Homo sapiens
DSGVTQTPKHLITATGQRVTLRCSPRSGDLSVYWYQQSLDQGLQFLIHYYNGEERAKGNILERFSAQQFPDLHSELNLSSLELGDSALYFCASSDSHTQYFGPGTRLTVL

SEQ ID NO: 423 - protein, 11 aa, Homo sapiens
CASSDSHTQYF

SEQ ID NO: 424 - protein, 17 aa, Homo sapiens
VYWYQQSLDQGLQFLIH

SEQ ID NO: 425 - DNA, 355 bp, Homo sapiens
  source 1..355: mol_type = other DNA
gctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagactccccatattcaggaggaggtgctgacggactcacctttggcaaagggactcatctaatcatccagccct

SEQ ID NO: 426 - protein, 118 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDSPYSGGGADGLTFGKGTHLIIQP

SEQ ID NO: 427 - protein, 6 aa, Homo sapiens
VSGNPY

SEQ ID NO: 428 - protein, 8 aa, Homo sapiens
YITGDNLV

SEQ ID NO: 429 - protein, 18 aa, Homo sapiens
CAVRDSPYSGGGADGLTF

SEQ ID NO: 430 - protein, 26 aa, Homo sapiens
AQSVAQPEDQVNVAEGNPLTVKCTYS

SEQ ID NO: 431 - protein, 17 aa, Homo sapiens
LFWYVQYPNRGLQFLLK

SEQ ID NO: 432 - protein, 33 aa, Homo sapiens
KGSYGFEAEFNKSQTSFHLKKPSALVSDSALYF

SEQ ID NO: 433 - DNA, 346 bp, Homo sapiens
  source 1..346: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgtcggtggaccgcgacagggcgaaaaactgttttttggcagtggaacccagctctctgtcttgg

SEQ ID NO: 434 - protein, 115 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVGGPRQGEKLFFGSGTQLSVL

SEQ ID NO: 435 - protein, 14 aa, Homo sapiens
CSVGGPRQGEKLFF

SEQ ID NO: 436 - DNA, 337 bp, Homo sapiens
  source 1..337: mol_type = other DNA
gatgctaagaccacccagcccccctccatggattgcgctgaaggaagagctgcaaacctgccttgtaatcactctaccatcagtggaaatgagtatgtgtattggtatcgacagattcactcccaggggccacagtatatcattcatggtctaaaaaacaatgaaaccaatgaaatggcctctctgatcatcacagaagacagaaagtccagcaccttgatcctgccccacgctacgctgagagacactgctgtgtactattgcatcgtcagattctcgtctggttctgcaaggcaactgacctttggatctgggacacaattgactgttttacctg

SEQ ID NO: 437 - protein, 112 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVRFSSGSARQLTFGSGTQLTVLP

SEQ ID NO: 438 - protein, 15 aa, Homo sapiens
CIVRFSSGSARQLTF

SEQ ID NO: 439 - protein, 25 aa, Homo sapiens
DAKTTQPPSMDCAEGRAANLPCNHS

SEQ ID NO: 440 - DNA, 346 bp, Homo sapiens
  source 1..346: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagctcccccgaaacggcgggcactgaagctttctttggacaaggcaccagactcacagttgtag

SEQ ID NO: 441 - protein, 115 aa, Homo sapiens
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSPETAGTEAFFGQGTRLTVV

SEQ ID NO: 442 - protein, 5 aa, Homo sapiens
SEHNR

SEQ ID NO: 443 - protein, 6 aa, Homo sapiens
FQNEAQ

SEQ ID NO: 444 - protein, 15 aa, Homo sapiens
CASSSPETAGTEAFF

SEQ ID NO: 445 - protein, 26 aa, Homo sapiens
DTGVSQNPRHKITKRGQNVTFRCDPI

SEQ ID NO: 446 - protein, 17 aa, Homo sapiens
LYWYRQTLGQGPEFLTY

SEQ ID NO: 447 - protein, 37 aa, Homo sapiens
LEKSRLLSDRFSAERPKGSFSTLEIQRTEQGDSAMYL

SEQ ID NO: 448 - DNA, 337 bp, Homo sapiens
  source 1..337: mol_type = other DNA
ggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagataggacgggaggaggaaacaaactcacctttgggacaggcactcagctaaaagtggaactca

SEQ ID NO: 449 - protein, 112 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVRDRTGGGNKLTFGTGTQLKVEL

SEQ ID NO: 450 - protein, 6 aa, Homo sapiens
TSGFNG

SEQ ID NO: 451 - protein, 6 aa, Homo sapiens
NVLDGL

SEQ ID NO: 452 - protein, 15 aa, Homo sapiens
CAVRDRTGGGNKLTF

SEQ ID NO: 453 - protein, 25 aa, Homo sapiens
GQNIDQPTEMTATEGAIVQINCTYQ

SEQ ID NO: 454 - protein, 17 aa, Homo sapiens
LFWYQQHAGEAPTFLSY

SEQ ID NO: 455 - protein, 33 aa, Homo sapiens
EEKGRFSSFLSRSKGYSYLLLKELQMKDSASYL

SEQ ID NO: 456 - protein, 10 aa, Homo sapiens
GTGTQLKVEL

SEQ ID NO: 457 - DNA, 337 bp, Homo sapiens
  source 1..337: mol_type = other DNA
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccacgagggaggctatggctacaccttcggttcggggaccaggttaaccgttgtag

SEQ ID NO: 458 - protein, 112 aa, Homo sapiens
DTAVSQTPKYLVTQMGNDKSIKCEQNLGHDTMYWYKQDSKKFLKIMFSYNNKELIINETVPNRFSPKSPDKAHLNLHINSLELGDSAVYFCASSHEGGYGYTFGSGTRLTVV

SEQ ID NO: 459 - protein, 13 aa, Homo sapiens
CASSHEGGYGYTF

SEQ ID NO: 460 - DNA, 337 bp, Homo sapiens
  source 1..337: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagcccgggacagggtttcgagcagtacttcgggccgggcaccaggctcacggtcacag

SEQ ID NO: 461 - protein, 112 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPGQGFEQYFGPGTRLTVT

SEQ ID NO: 462 - protein, 13 aa, Homo sapiens
CASSPGQGFEQYF

SEQ ID NO: 463 - DNA, 337 bp, Homo sapiens
  source 1..337: mol_type = other DNA
aaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgttcccgataactatggtcagaattttgtctttggtcccggaaccagattgtccgtgctgccct

SEQ ID NO: 464 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVPDNYGQNFVFGPGTRLSVLP

SEQ ID NO: 465 - protein, 13 aa, Homo sapiens
CAVPDNYGQNFVF

SEQ ID NO: 466 - DNA, 337 bp, Homo sapiens
  source 1..337: mol_type = other DNA
gatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttaaacgggagagagctgttttttggagaaggctctaggctgaccgtactgg

SEQ ID NO: 467 - protein, 112 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLNGRELFFGEGSRLTVL

SEQ ID NO: 468 - protein, 5 aa, Homo sapiens
SGHDY

SEQ ID NO: 469 - protein, 6 aa, Homo sapiens
FNNNVP

SEQ ID NO: 470 - protein, 12 aa, Homo sapiens
CASSLNGRELFF

SEQ ID NO: 471 - protein, 26 aa, Homo sapiens
DAGVIQSPRHEVTEMGQEVTLRCKPI

SEQ ID NO: 472 - protein, 17 aa, Homo sapiens
LFWYRQTMMRGLELLIY

SEQ ID NO: 473 - protein, 37 aa, Homo sapiens
IDDSGMPEDRFSAKMPNASFSTLKIQPSEPRDSAVYF

SEQ ID NO: 474 - DNA, 337 bp, Homo sapiens
  source 1..337: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcctcactagggggatagtgggctacaccttcggttcggggaccaggttaaccgttgtag

SEQ ID NO: 475 - protein, 112 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASLTRGIVGYTFGSGTRLTVV

SEQ ID NO: 476 - protein, 13 aa, Homo sapiens
CASLTRGIVGYTF

SEQ ID NO: 477 - DNA, 352 bp, Homo sapiens
  source 1..352: mol_type = other DNA
gctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagacgggagctatggtggtgctacaaacaagctcatctttggaactggcactctgcttgctgtccagccaa

SEQ ID NO: 478 - protein, 117 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRDGSYGGATNKLIFGTGTLLAVQP

SEQ ID NO: 479 - protein, 17 aa, Homo sapiens
CAVRDGSYGGATNKLIF

SEQ ID NO: 480 - protein, 10 aa, Homo sapiens
GTGTLLAVQP

SEQ ID NO: 481 - DNA, 349 bp, Homo sapiens
  source 1..349: mol_type = other DNA
gaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagactcggccctgtatctctgtgccagcagccgtttgggactagcggggttacgggagacccagtacttcgggccaggcacgcggctcctggtgctcg

SEQ ID NO: 482 - protein, 116 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSRLGLAGLRETQYFGPGTRLLVL

SEQ ID NO: 483 - protein, 17 aa, Homo sapiens
CASSRLGLAGLRETQYF

SEQ ID NO: 484 - DNA, 364 bp, Homo sapiens
  source 1..364: mol_type = other DNA
gcccagacagtcactcagtctcaaccagagatgtctgtgcaggaggcagagactgtgaccctgagttgcacatatgacaccagtgagaataattattatttgttctggtacaagcagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgaccctccctattactcggtctggggctgggagttaccaactcactttcgggaaggggaccaaactctcggtcataccaa

SEQ ID NO: 485 - protein, 121 aa, Homo sapiens
AQTVTQSQPEMSVQEAETVTLSCTYDTSENNYYLFWYKQPPSRQMILVIRQEAYKQQNATENRFSVNFQKAAKSFSLKISDSQLGDTAMYFCAFMTLPITRSGAGSYQLTFGKGTKLSVIP

SEQ ID NO: 486 - protein, 7 aa, Homo sapiens
TSENNYY

SEQ ID NO: 487 - protein, 20 aa, Homo sapiens
CAFMTLPITRSGAGSYQLTF

SEQ ID NO: 488 - protein, 33 aa, Homo sapiens
ATENRFSVNFQKAAKSFSLKISDSQLGDTAMYF

SEQ ID NO: 489 - protein, 10 aa, Homo sapiens
GKGTKLSVIP

SEQ ID NO: 490 - DNA, 343 bp, Homo sapiens
  source 1..343: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcaggttcacagggggatataactacgagcagtacttcgggccgggcaccaggctcacggtcacag

SEQ ID NO: 491 - protein, 114 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASRFTGGYNYEQYFGPGTRLTVT

SEQ ID NO: 492 - protein, 15 aa, Homo sapiens
CASRFTGGYNYEQYF

SEQ ID NO: 493 - DNA, 355 bp, Homo sapiens
  source 1..355: mol_type = other DNA
gcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttaaaggcatcaacggttttgaggctgaatttaacaagagtcaaacttccttccacttgaggaaaccctcagtccatataagcgacacggctgagtacttctgtgctgtgattgagggagaggctggtggtactagctatggaaagctgacatttggacaagggaccatcttgactgtccatccaa

SEQ ID NO: 494 - protein, 118 aa, Homo sapiens
AQSVTQLDSQVPVFEEAPVELRCNYSSSVSVYLFWYVQYPNQGLQLLLKYLSGSTLVKGINGFEAEFNKSQTSFHLRKPSVHISDTAEYFCAVIEGEAGGTSYGKLTFGQGTILTVHP

SEQ ID NO: 495 - protein, 6 aa, Homo sapiens
SSVSVY

SEQ ID NO: 496 - protein, 8 aa, Homo sapiens
YLSGSTLV

SEQ ID NO: 497 - protein, 18 aa, Homo sapiens
CAVIEGEAGGTSYGKLTF

SEQ ID NO: 498 - protein, 26 aa, Homo sapiens
AQSVTQLDSQVPVFEEAPVELRCNYS

SEQ ID NO: 499 - protein, 17 aa, Homo sapiens
LFWYVQYPNQGLQLLLK

SEQ ID NO: 500 - protein, 33 aa, Homo sapiens
KGINGFEAEFNKSQTSFHLRKPSVHISDTAEYF

SEQ ID NO: 501 - DNA, 334 bp, Homo sapiens
  source 1..334: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtcctcacgccggggagctgttttttggagaaggctctaggctgaccgtactgg

SEQ ID NO: 502 - protein, 111 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSPHAGELFFGEGSRLTVL

SEQ ID NO: 503 - protein, 12 aa, Homo sapiens
CASSPHAGELFF

SEQ ID NO: 504 - DNA, 337 bp, Homo sapiens
  source 1..337: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtcccaccgatgacaactgacagctgggggaaattgcagtttggagcagggacccaggttgtggtcaccccag

SEQ ID NO: 505 - protein, 112 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVPPMTTDSWGKLQFGAGTQVVVTP

SEQ ID NO: 506 - protein, 5 aa, Homo sapiens
DSVNN

SEQ ID NO: 507 - protein, 5 aa, Homo sapiens
IPSGT

SEQ ID NO: 508 - protein, 16 aa, Homo sapiens
CAVPPMTTDSWGKLQF

SEQ ID NO: 509 - protein, 26 aa, Homo sapiens
GIQVEQSPPDLILQEGANSTLRCNFS

SEQ ID NO: 510 - protein, 17 aa, Homo sapiens
LQWFHQNPWGQLINLFY

SEQ ID NO: 511 - protein, 33 aa, Homo sapiens
KQNGRLSATTVATERYSLLYISSSQTTDSGVYF

SEQ ID NO: 512 - DNA, 343 bp, Homo sapiens
  source 1..343: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttacggcgggggcaatcctacgagcagtacttcgggccgggcaccaggctcacggtcacag

SEQ ID NO: 513 - protein, 114 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLRRGQSYEQYFGPGTRLTVT

SEQ ID NO: 514 - protein, 5 aa, Homo sapiens
SGHRS

SEQ ID NO: 515 - protein, 6 aa, Homo sapiens
YFSETQ

SEQ ID NO: 516 - protein, 15 aa, Homo sapiens
CASSLRRGQSYEQYF

SEQ ID NO: 517 - protein, 26 aa, Homo sapiens
KAGVTQTPRYLIKTRGQQVTLSCSPI

SEQ ID NO: 518 - protein, 17 aa, Homo sapiens
VSWYQQTPGQGLQFLFE

SEQ ID NO: 519 - protein, 36 aa, Homo sapiens
RNKGNFPGRFSGRQFSNSRSEMNVSTLELGDSALYL

SEQ ID NO: 520 - DNA, 325 bp, Homo sapiens
  source 1..325: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctggatgcaggcaacatgctcacctttggagggggaacaaggttaatggtcaaacccc

SEQ ID NO: 521 - protein, 108 aa, Homo sapiens
DAKTTQPNSMESNEEEPVHLPCNHSTISGTDYIHWYRQLPSQGPEYVIHGLTSNVNNRMASLAIAEDRKSSTLILHRATLRDAAVYYCILDAGNMLTFGGGTRLMVKP

SEQ ID NO: 522 - protein, 11 aa, Homo sapiens
CILDAGNMLTF

SEQ ID NO: 523 - DNA, 343 bp, Homo sapiens
  source 1..343: mol_type = other DNA
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcaaggagggttggggggacactgaagctttctttggacaaggcaccagactcacagttgtag

SEQ ID NO: 524 - protein, 114 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASKEGWGDTEAFFGQGTRLTVV

SEQ ID NO: 525 - protein, 5 aa, Homo sapiens
SNHLY

SEQ ID NO: 526 - protein, 6 aa, Homo sapiens
FYNNEI

SEQ ID NO: 527 - protein, 14 aa, Homo sapiens
CASKEGWGDTEAFF

SEQ ID NO: 528 - protein, 26 aa, Homo sapiens
EPEVTQTPSHQVTQMGQEVILRCVPI

SEQ ID NO: 529 - protein, 17 aa, Homo sapiens
FYWYRQILGQKVEFLVS

SEQ ID NO: 530 - protein, 37 aa, Homo sapiens
SEKSEIFDDQFSVERPDGSNFTLKIRSTKLEDSAMYF

SEQ ID NO: 531 - DNA, 337 bp, Homo sapiens
  source 1..337: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggagtaggaatggatagcagctataaattgatcttcgggagtgggaccagactgctggtcaggcctg

SEQ ID NO: 532 - protein, 112 aa, Homo sapiens
TQLLEQSPQFLSIQEGENLTVYCNSSSVFSSLQWYRQEPGEGPVLLVTVVTGGEVKKLKRLTFQFGDARKDSSLHITAAQPGDTGLYLCAGVGMDSSYKLIFGSGTRLLVRP

SEQ ID NO: 533 - protein, 5 aa, Homo sapiens
SVFSS

SEQ ID NO: 534 - protein, 7 aa, Homo sapiens
VVTGGEV

SEQ ID NO: 535 - protein, 14 aa, Homo sapiens
CAGVGMDSSYKLIF

SEQ ID NO: 536 - protein, 26 aa, Homo sapiens
TQLLEQSPQFLSIQEGENLTVYCNSS

SEQ ID NO: 537 - protein, 17 aa, Homo sapiens
LQWYRQEPGEGPVLLVT

SEQ ID NO: 538 - protein, 33 aa, Homo sapiens
KKLKRLTFQFGDARKDSSLHITAAQPGDTGLYL

SEQ ID NO: 539 - protein, 10 aa, Homo sapiens
GSGTRLLVRP

SEQ ID NO: 540 - DNA, 340 bp, Homo sapiens
  source 1..340: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttcccgacagggttatcagccccagcattttggtgatgggactcgactctccatcctag

SEQ ID NO: 541 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSSRQGYQPQHFGDGTRLSIL

SEQ ID NO: 542 - protein, 14 aa, Homo sapiens
CASSSRQGYQPQHF

SEQ ID NO: 543 - DNA, 340 bp, Homo sapiens
  source 1..340: mol_type = other DNA
gctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagcggaaacacacctcttgtctttggaaagggcacaagactttctgtgattgcaa

SEQ ID NO: 544 - protein, 113 aa, Homo sapiens
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRSGNTPLVFGKGTRLSVIA

SEQ ID NO: 545 - protein, 12 aa, Homo sapiens
CAYRSGNTPLVF

SEQ ID NO: 546 - protein, 10 aa, Homo sapiens
GKGTRLSVIA

SEQ ID NO: 547 - DNA, 346 bp, Homo sapiens
  source 1..346: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactcgctgctgtcgccctcctacgagcagtacttcgggccgggcaccaggctcacggtcacag

SEQ ID NO: 548 - protein, 115 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSLLSPSYEQYFGPGTRLTVT

SEQ ID NO: 549 - protein, 16 aa, Homo sapiens
CASSYSLLSPSYEQYF

SEQ ID NO: 550 - DNA, 337 bp, Homo sapiens
  source 1..337: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggaccaccgaactagctatggaaagctgacatttggacaagggaccatcttgactgtccatccaa

SEQ ID NO: 551 - protein, 112 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDHRTSYGKLTFGQGTILTVHP

SEQ ID NO: 552 - protein, 5 aa, Homo sapiens
TSINN

SEQ ID NO: 553 - protein, 7 aa, Homo sapiens
IRSNERE

SEQ ID NO: 554 - protein, 14 aa, Homo sapiens
CATDHRTSYGKLTF

SEQ ID NO: 555 - protein, 26 aa, Homo sapiens
SQQGEEDPQALSIQEGENATMNCSYK

SEQ ID NO: 556 - protein, 17 aa, Homo sapiens
LQWYRQNSGRGLVHLIL

SEQ ID NO: 557 - protein, 33 aa, Homo sapiens
KHSGRLRVTLDTSKKSSSLLITASRAADTASYF

SEQ ID NO: 558 - DNA, 340 bp, Homo sapiens
  source 1..340: mol_type = other DNA
gagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagctcagaacggggacataagggctacaccttcggttcggggaccaggttaaccgttgtag

SEQ ID NO: 559 - protein, 113 aa, Homo sapiens
ETGVTQSPTHLIKTRGQQVTLRCSSQSGHNTVSWYQQALGQGPQFIFQYYREEENGRGNFPPRFSGLQFPNYSSELNVNALELDDSALYLCASSSERGHKGYTFGSGTRLTVV

SEQ ID NO: 560 - protein, 14 aa, Homo sapiens
CASSSERGHKGYTF

SEQ ID NO: 561 - DNA, 337 bp, Homo sapiens
  source 1..337: mol_type = other DNA
aaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtgttatccggtggtgctacaaacaagctcatctttggaactggcactctgcttgctgtccagccaa

SEQ ID NO: 562 - protein, 112 aa, Homo sapiens
KNQVEQSPQSLIILEGKNCTLQCNYTVSPFSNLRWYKQDTGRGPVSLTIMTFSENTKSNGRYTATLDADTKQSSLHITASQLSDSASYICVLSGGATNKLIFGTGTLLAVQP

SEQ ID NO: 563 - protein, 13 aa, Homo sapiens
CVLSGGATNKLIF

SEQ ID NO: 564 - DNA, 343 bp, Homo sapiens
  source 1..343: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtacgactagcgggagtttggagacccagtacttcgggccaggcacgcggctcctggtgctcg

SEQ ID NO: 565 - protein, 114 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSTTSGSLETQYFGPGTRLLVL

SEQ ID NO: 566 - protein, 15 aa, Homo sapiens
CASSTTSGSLETQYF

SEQ ID NO: 567 - DNA, 343 bp, Homo sapiens
  source 1..343: mol_type = other DNA
aaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtggtgagcgcgggcagggaatatggaaacaagctggtctttggcgcaggaaccattctgagagtcaagtcct

SEQ ID NO: 568 - protein, 114 aa, Homo sapiens
KNQVEQSPQSLIILEGKNCTLQCNYTVSPFSNLRWYKQDTGRGPVSLTIMTFSENTKSNGRYTATLDADTKQSSLHITASQLSDSASYICVVSAGREYGNKLVFGAGTILRVKS

SEQ ID NO: 569 - protein, 15 aa, Homo sapiens
CVVSAGREYGNKLVF

SEQ ID NO: 570 - DNA, 340 bp, Homo sapiens
  source 1..340: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagttcgacaggcatgaacactgaagctttctttggacaaggcaccagactcacagttgtag

SEQ ID NO: 571 - protein, 113 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSSTGMNTEAFFGQGTRLTVV

SEQ ID NO: 572 - protein, 14 aa, Homo sapiens
CASSSTGMNTEAFF

SEQ ID NO: 573 - DNA, 337 bp, Homo sapiens
  source 1..337: mol_type = other DNA
ggacaacaggtaatgcaaattcctcagtaccagcatgtacaagaaggagaagacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcagggctaactcgggatagcaactatcagttaatctggggcgctgggaccaagctaattataaagccag

SEQ ID NO: 574 - protein, 112 aa, Homo sapiens
GQQVMQIPQYQHVQEGEDFTTYCNSSTTLSNIQWYKQRPGGHPVFLIQLVKSGEVKKQKRLTFQFGEAKKNSSLHITATQTTDVGTYFCAGLTRDSNYQLIWGAGTKLIIKP

SEQ ID NO: 575 - protein, 5 aa, Homo sapiens
TTLSN

SEQ ID NO: 576 - protein, 7 aa, Homo sapiens
LVKSGEV

SEQ ID NO: 577 - protein, 14 aa, Homo sapiens
CAGLTRDSNYQLIW

SEQ ID NO: 578 - protein, 26 aa, Homo sapiens
GQQVMQIPQYQHVQEGEDFTTYCNSS

SEQ ID NO: 579 - protein, 17 aa, Homo sapiens
IQWYKQRPGGHPVFLIQ

SEQ ID NO: 580 - protein, 33 aa, Homo sapiens
KKQKRLTFQFGEAKKNSSLHITATQTTDVGTYF

SEQ ID NO: 581 - DNA, 334 bp, Homo sapiens
  source 1..334: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagttcgtccccctttggctacaccttcggttcggggaccaggttaaccgttgtag

SEQ ID NO: 582 - protein, 111 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSSPFGYTFGSGTRLTVV

SEQ ID NO: 583 - protein, 12 aa, Homo sapiens
CASSSSPFGYTF

SEQ ID NO: 584 - DNA, 340 bp, Homo sapiens
  source 1..340: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgagcgatacctcaggaacctacaaatacatctttggaacaggcaccaggctgaaggttttagcaa

SEQ ID NO: 585 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVSDTSGTYKYIFGTGTRLKVLA

SEQ ID NO: 586 - protein, 14 aa, Homo sapiens
CAVSDTSGTYKYIF

SEQ ID NO: 587 - protein, 10 aa, Homo sapiens
GTGTRLKVLA

SEQ ID NO: 588 - DNA, 346 bp, Homo sapiens
  source 1..346: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccacgtcgatgggggcggtgagacccagtacttcgggccaggcacgcggctcctggtgctcg

SEQ ID NO: 589 - protein, 115 aa, Homo sapiens
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSHVDGGGETQYFGPGTRLLVL

SEQ ID NO: 590 - protein, 15 aa, Homo sapiens
CASSHVDGGGETQYF

SEQ ID NO: 591 - protein, 26 aa, Homo sapiens
DTGVSQDPRHKITKRGQNVTFRCDPI

SEQ ID NO: 592 - DNA, 328 bp, Homo sapiens
  source 1..328: mol_type = other DNA
cttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgctacaggaggaagctacatacctacatttggaagaggaaccagccttattgttcatccgt

SEQ ID NO: 593 - protein, 109 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLATGGSYIPTFGRGTSLIVHP

SEQ ID NO: 594 - protein, 7 aa, Homo sapiens
NIATNDY

SEQ ID NO: 595 - protein, 5 aa, Homo sapiens
GYKTK

SEQ ID NO: 596 - protein, 12 aa, Homo sapiens
CLATGGSYIPTF

SEQ ID NO: 597 - protein, 25 aa, Homo sapiens
LAKTTQPISMDSYEGQEVNITCSHN

SEQ ID NO: 598 - protein, 17 aa, Homo sapiens
ITWYQQFPSQGPRFIIQ

SEQ ID NO: 599 - protein, 33 aa, Homo sapiens
VTNEVASLFIPADRKSSTLSLPRVSLSDTAVYY

SEQ ID NO: 600 - protein, 10 aa, Homo sapiens
GRGTSLIVHP

SEQ ID NO: 601 - DNA, 349 bp, Homo sapiens
  source 1..349: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgaaatggggcggagctcctacaatgagcagttcttcgggccagggacacggctcaccgtgctag

SEQ ID NO: 602 - protein, 116 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVEMGRSSYNEQFFGPGTRLTVL

SEQ ID NO: 603 - protein, 15 aa, Homo sapiens
CSVEMGRSSYNEQFF

SEQ ID NO: 604 - DNA, 325 bp, Homo sapiens
  source 1..325: mol_type = other DNA
ggacaacaggtaatgcaaattcctcagtaccagcatgtacaagaaggagaagacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcagggacgtatgacaagatcatctttggaaaagggacacgacttcatattctcccca

SEQ ID NO: 605 - protein, 108 aa, Homo sapiens
GQQVMQIPQYQHVQEGEDFTTYCNSSTTLSNIQWYKQRPGGHPVFLIQLVKSGEVKKQKRLTFQFGEAKKNSSLHITATQTTDVGTYFCAGTYDKIIFGKGTRLHILP

SEQ ID NO: 606 - protein, 10 aa, Homo sapiens
CAGTYDKIIF

SEQ ID NO: 607 - protein, 10 aa, Homo sapiens
GKGTRLHILP

SEQ ID NO: 608 - DNA, 337 bp, Homo sapiens
  source 1..337: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatcaaccccctacgagcagtacttcgggccgggcaccaggctcacggtcacag

SEQ ID NO: 609 - protein, 112 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLSTPYEQYFGPGTRLTVT

SEQ ID NO: 610 - protein, 13 aa, Homo sapiens
CASSLSTPYEQYF

SEQ ID NO: 611 - DNA, 346 bp, Homo sapiens
  source 1..346: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcggggtggcaggtgaatatggaaacaagctggtctttggcgcaggaaccattctgagagtcaagtcct

SEQ ID NO: 612 - protein, 115 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASGVAGEYGNKLVFGAGTILRVKS

SEQ ID NO: 613 - protein, 16 aa, Homo sapiens
CAASGVAGEYGNKLVF

SEQ ID NO: 614 - DNA, 343 bp, Homo sapiens
  source 1..343: mol_type = other DNA
actgctgggatcacccaggcaccaacatctcagatcctggcagcaggacggagcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttctgtgccagcagcgcgggacagggggccggcgatgagcagttcttcgggccagggacacggctcaccgtgctag

SEQ ID NO: 615 - protein, 114 aa, Homo sapiens
TAGITQAPTSQILAAGRSMTLRCTQDMRHNAMYWYRQDLGLGLRLIHYSNTAGTTGKGEVPDGYSVSRANTDDFPLTLASAVPSQTSVYFCASSAGQGAGDEQFFGPGTRLTVL

SEQ ID NO: 616 - protein, 15 aa, Homo sapiens
CASSAGQGAGDEQFF

SEQ ID NO: 617 - DNA, 346 bp, Homo sapiens
  source 1..346: mol_type = other DNA
ggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagatccccgcatggctctggggctgggagttaccaactcactttcgggaaggggaccaaactctcggtcataccaa

SEQ ID NO: 618 - protein, 115 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVRSPHGSGAGSYQLTFGKGTKLSVIP

SEQ ID NO: 619 - protein, 18 aa, Homo sapiens
CAVRSPHGSGAGSYQLTF

SEQ ID NO: 620 - DNA, 343 bp, Homo sapiens
  source 1..343: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcgacgacggtagctcctacgagcagtacttcgggccgggcaccaggctcacggtcacag

SEQ ID NO: 621 - protein, 114 aa, Homo sapiens
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSDDGSSYEQYFGPGTRLTVT

SEQ ID NO: 622 - protein, 14 aa, Homo sapiens
CASSDDGSSYEQYF

SEQ ID NO: 623 - DNA, 346 bp, Homo sapiens
  source 1..346: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcggaggggtaggcaccaatgcaggcaaatcaacctttggggatgggactacgctcactgtgaagccaa

SEQ ID NO: 624 - protein, 115 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASGGVGTNAGKSTFGDGTTLTVKP

SEQ ID NO: 625 - protein, 16 aa, Homo sapiens
CAASGGVGTNAGKSTF

SEQ ID NO: 626 - protein, 10 aa, Homo sapiens
GDGTTLTVKP

SEQ ID NO: 627 - DNA, 349 bp, Homo sapiens
  source 1..349: mol_type = other DNA
ggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcagcttcgggggggacccacgcactcagccccagcattttggtgatgggactcgactctccatcctag

SEQ ID NO: 628 - protein, 116 aa, Homo sapiens
GAGVSQSPSNKVTEKGKDVELRCDPISGHTALYWYRQSLGQGLEFLIYFQGNSAPDKSGLPSDRFSAERTGGSVSTLTIQRTQQEDSAVYLCASSFGGDPRTQPQHFGDGTRLSIL

SEQ ID NO: 629 - protein, 6 aa, Homo sapiens
FQGNSA

SEQ ID NO: 630 - protein, 16 aa, Homo sapiens
CASSFGGDPRTQPQHF

SEQ ID NO: 631 - protein, 26 aa, Homo sapiens
GAGVSQSPSNKVTEKGKDVELRCDPI

SEQ ID NO: 632 - protein, 17 aa, Homo sapiens
LYWYRQSLGQGLEFLIY

SEQ ID NO: 633 - protein, 37 aa, Homo sapiens
PDKSGLPSDRFSAERTGGSVSTLTIQRTQQEDSAVYL

SEQ ID NO: 634 - DNA, 337 bp, Homo sapiens
  source 1..337: mol_type = other DNA
cttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgggtctatctaactttggaaatgagaaattaacctttgggactggaacaagactcaccatcataccca

SEQ ID NO: 635 - protein, 112 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGLSNFGNEKLTFGTGTRLTIIP

SEQ ID NO: 636 - protein, 15 aa, Homo sapiens
CLVGLSNFGNEKLTF

SEQ ID NO: 637 - DNA, 343 bp, Homo sapiens
  source 1..343: mol_type = other DNA
gacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaaggtcagggtctgaacactgaagctttctttggacaaggcaccagactcacagttgtag

SEQ ID NO: 638 - protein, 114 aa, Homo sapiens
DTEVTQTPKHLVMGMTNKKSLKCEQHMGHRAMYWYKQKAKKPPELMFVYSYEKLSINESVPSRFSPECPNSSLLNLHLHALQPEDSALYLCASSQGQGLNTEAFFGQGTRLTVV

SEQ ID NO: 639 - protein, 5 aa, Homo sapiens
MGHRA

SEQ ID NO: 640 - protein, 6 aa, Homo sapiens
YSYEKL

SEQ ID NO: 641 - protein, 15 aa, Homo sapiens
CASSQGQGLNTEAFF

SEQ ID NO: 642 - protein, 26 aa, Homo sapiens
DTEVTQTPKHLVMGMTNKKSLKCEQH

SEQ ID NO: 643 - protein, 17 aa, Homo sapiens
MYWYKQKAKKPPELMFV

SEQ ID NO: 644 - protein, 36 aa, Homo sapiens
SINESVPSRFSPECPNSSLLNLHLHALQPEDSALYL

SEQ ID NO: 645 - DNA, 340 bp, Homo sapiens
  source 1..340: mol_type = other DNA
ggacaaaacattgaccagcccactgagatgacagctacggaaggtgccattgtccagatcaactgcacgtaccagacatctgggttcaacgggctgttctggtaccagcaacatgctggcgaagcacctacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagcatactctggggctgggagttaccaactcactttcgggaaggggaccaaactctcggtcataccaa

SEQ ID NO: 646 - protein, 113 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVRAYSGAGSYQLTFGKGTKLSVIP

SEQ ID NO: 647 - protein, 16 aa, Homo sapiens
CAVRAYSGAGSYQLTF

SEQ ID NO: 648 - DNA, 352 bp, Homo sapiens
  source 1..352: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagattggggcagggccccagatacgcagtattttggcccaggcacccggctgacagtgctcg

SEQ ID NO: 649 - protein, 117 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDWGRAPDTQYFGPGTRLTVL

SEQ ID NO: 650 - protein, 15 aa, Homo sapiens
CSARDWGRAPDTQYF

SEQ ID NO: 651 - DNA, 331 bp, Homo sapiens
  source 1..331: mol_type = other DNA
ggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagattaacaccggtaaccagttctattttgggacagggacaagtttgacggtcattccaa

SEQ ID NO: 652 - protein, 110 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAEINTGNQFYFGTGTSLTVIP

SEQ ID NO: 653 - protein, 12 aa, Homo sapiens
CAEINTGNQFYF

SEQ ID NO: 654 - DNA, 343 bp, Homo sapiens
  source 1..343: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgcacaaggggacaggggggatggagctttctttggacaaggcaccagactcacagttgtag

SEQ ID NO: 655 - protein, 114 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSAQGDRGDGAFFGQGTRLTVV

SEQ ID NO: 656 - protein, 15 aa, Homo sapiens
CASSAQGDRGDGAFF

SEQ ID NO: 657 - DNA, 346 bp, Homo sapiens
  source 1..346: mol_type = other DNA
gcccagaagataactcaaacccaaccaggaatgttcgtgcaggaaaaggaggctgtgactctggactgcacatatgacaccagtgatccaagttatggtctattctggtacaagcagcccagcagtggggaaatgatttttcttatttatcaggggtcttatgaccagcaaaatgcaacagaaggtcgctactcattgaatttccagaaggcaagaaaatccgccaaccttgtcatctccgcttcacaactgggggactcagcaatgtatttctgtgcaatgagagagacgggtagcaactataaactgacatttggaaaaggaactctcttaaccgtgaatccaa

SEQ ID NO: 658 - protein, 115 aa, Homo sapiens
AQKITQTQPGMFVQEKEAVTLDCTYDTSDPSYGLFWYKQPSSGEMIFLIYQGSYDQQNATEGRYSLNFQKARKSANLVISASQLGDSAMYFCAMRETGSNYKLTFGKGTLLTVNP

SEQ ID NO: 659 - protein, 7 aa, Homo sapiens
TSDPSYG

SEQ ID NO: 660 - protein, 8 aa, Homo sapiens
QGSYDQQN

SEQ ID NO: 661 - protein, 14 aa, Homo sapiens
CAMRETGSNYKLTF

SEQ ID NO: 662 - protein, 10 aa, Homo sapiens
GKGTLLTVNP

SEQ ID NO: 663 - DNA, 337 bp, Homo sapiens
  source 1..337: mol_type = other DNA
gctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagcttagcaggggagggtgaagctttctttggacaaggcaccagactcacagttgtag

SEQ ID NO: 664 - protein, 112 aa, Homo sapiens
AAGVIQSPRHLIKEKRETATLKCYPIPRHDTVYWYQQGPGQDPQFLISFYEKMQSDKGSIPDRFSAQQFSDYHSELNMSSLELGDSALYFCASSLAGEGEAFFGQGTRLTVV

SEQ ID NO: 665 - protein, 5 aa, Homo sapiens
PRHDT

SEQ ID NO: 666 - protein, 6 aa, Homo sapiens
FYEKMQ

SEQ ID NO: 667 - protein, 13 aa, Homo sapiens
CASSLAGEGEAFF

SEQ ID NO: 668 - protein, 26 aa, Homo sapiens
AAGVIQSPRHLIKEKRETATLKCYPI

SEQ ID NO: 669 - protein, 17 aa, Homo sapiens
VYWYQQGPGQDPQFLIS

SEQ ID NO: 670 - protein, 36 aa, Homo sapiens
SDKGSIPDRFSAQQFSDYHSELNMSSLELGDSALYF

SEQ ID NO: 671 - DNA, 331 bp, Homo sapiens
  source 1..331: mol_type = other DNA
cagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcagtcgggggcggatctgaaaagctggtctttggaaagggaacgaaactgacagtaaacccat

SEQ ID NO: 672 - protein, 110 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAVGGGSEKLVFGKGTKLTVNP

SEQ ID NO: 673 - protein, 6 aa, Homo sapiens
NSAFQY

SEQ ID NO: 674 - protein, 6 aa, Homo sapiens
TYSSGN

SEQ ID NO: 675 - protein, 12 aa, Homo sapiens
CAVGGGSEKLVF

SEQ ID NO: 676 - protein, 26 aa, Homo sapiens
QKEVEQDPGPLSVPEGAIVSLNCTYS

SEQ ID NO: 677 - protein, 17 aa, Homo sapiens
FMWYRQYSRKGPELLMY

SEQ ID NO: 678 - protein, 33 aa, Homo sapiens
KEDGRFTAQVDKSSKYISLFIRDSQPSDSATYL

SEQ ID NO: 679 - DNA, 340 bp, Homo sapiens
  source 1..340: mol_type = other DNA
gatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagttggacagaagagacctacgagcagtacttcgggccgggcaccaggctcacggtcacag

SEQ ID NO: 680 - protein, 113 aa, Homo sapiens
DVKVTQSSRYLVKRTGEKVFLECVQDMDHENMFWYRQDPGLGLRLIYFSYDVKMKEKGDIPEGYSVSREKKERFSLILESASTNQTSMYLCASSWTEETYEQYFGPGTRLTVT

SEQ ID NO: 681 - protein, 14 aa, Homo sapiens
CASSWTEETYEQYF

SEQ ID NO: 682 - DNA, 340 bp, Homo sapiens
  source 1..340: mol_type = other DNA
gctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagcgtacaataacaatgacatgcgctttggagcagggaccagactgacagtaaaaccaa

SEQ ID NO: 683 - protein, 113 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAYNNNDMRFGAGTRLTVKP

SEQ ID NO: 684 - protein, 13 aa, Homo sapiens
CAVRAYNNNDMRF

SEQ ID NO: 685 - protein, 10 aa, Homo sapiens
GAGTRLTVKP

SEQ ID NO: 686 - DNA, 340 bp, Homo sapiens
  source 1..340: mol_type = other DNA
gatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttagggacaggtagtgagcagttcttcgggccagggacacggctcaccgtgctag

SEQ ID NO: 687 - protein, 113 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLGTGSEQFFGPGTRLTVL

SEQ ID NO: 688 - protein, 13 aa, Homo sapiens
CASSLGTGSEQFF

SEQ ID NO: 689 - DNA, 340 bp, Homo sapiens
  source 1..340: mol_type = other DNA
aaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtacgtaccatttcagatggccagaagctgctctttgcaagggggaccatgttaaaggtggatctta

SEQ ID NO: 690 - protein, 113 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRTISDGQKLLFARGTMLKVDL

SEQ ID NO: 691 - protein, 14 aa, Homo sapiens
CAVRTISDGQKLLF

SEQ ID NO: 692 - DNA, 343 bp, Homo sapiens
  source 1..343: mol_type = other DNA
gctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagccggatacgggggggaatgaacactgaagctttctttggacaaggcaccagactcacagttgtag

SEQ ID NO: 693 - protein, 114 aa, Homo sapiens
AAGVIQSPRHLIKEKRETATLKCYPIPRHDTVYWYQQGPGQDPQFLISFYEKMQSDKGSIPDRFSAQQFSDYHSELNMSSLELGDSALYFCASRIRGGMNTEAFFGQGTRLTVV

SEQ ID NO: 694 - protein, 15 aa, Homo sapiens
CASRIRGGMNTEAFF

SEQ ID NO: 695 - DNA, 346 bp, Homo sapiens
  source 1..346: mol_type = other DNA
gctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagagctcctctttcagatggccagaagctgctctttgcaaggggaaccatgttaaaggtggatctta

SEQ ID NO: 696 - protein, 115 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPLSDGQKLLFARGTMLKVDL

SEQ ID NO: 697 - protein, 15 aa, Homo sapiens
CAVRAPLSDGQKLLF

SEQ ID NO: 698 - DNA, 358 bp, Homo sapiens
  source 1..358: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagacaggactagcggggggagctcctacgagcagtacttcgggccgggcaccaggctcacggtcacag

SEQ ID NO: 699 - protein, 119 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDRTSGGSSYEQYFGPGTRLTVT

SEQ ID NO: 700 - protein, 17 aa, Homo sapiens
CSARDRTSGGSSYEQYF

SEQ ID NO: 701 - DNA, 325 bp, Homo sapiens
  source 1..325: mol_type = other DNA
cttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtgggttccctcaacaaactggtctttggcgcaggaaccattctgagagtcaagtcct

SEQ ID NO: 702 - protein, 108 aa, Homo sapiens
LAKTTQPISMDSYEGQEVNITCSHNNIATNDYITWYQQFPSQGPRFIIQGYKTKVTNEVASLFIPADRKSSTLSLPRVSLSDTAVYYCLVGSLNKLVFGAGTILRVKS

SEQ ID NO: 703 - protein, 11 aa, Homo sapiens
CLVGSLNKLVF

SEQ ID NO: 704 - DNA, 349 bp, Homo sapiens
  source 1..349: mol_type = other DNA
gaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaatgggggggcgggagctcctacaatgagcagttcttcgggccagggacacggctcaccgtgctag

SEQ ID NO: 705 - protein, 116 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQWGGGSSYNEQFFGPGTRLTVL

SEQ ID NO: 706 - protein, 6 aa, Homo sapiens
YSLEER

SEQ ID NO: 707 - protein, 17 aa, Homo sapiens
CASSQWGGGSSYNEQFF

SEQ ID NO: 708 - protein, 36 aa, Homo sapiens
VENNSVPSRFSPECPNSSHLFLHLHTLQPEDSALYL

SEQ ID NO: 709 - DNA, 328 bp, Homo sapiens
  source 1..328: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtgattacctcaggaacctacaaatacatctttggaacaggcaccaggctgaaggttttagcaa

SEQ ID NO: 710 - protein, 109 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVITSGTYKYIFGTGTRLKVLA

SEQ ID NO: 711 - protein, 13 aa, Homo sapiens
CAVITSGTYKYIF

SEQ ID NO: 712 - DNA, 340 bp, Homo sapiens
  source 1..340: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagtacgaccgggacactctacgagcagtacttcgggccgggcaccaggctcacggtcacag

SEQ ID NO: 713 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSTTGTLYEQYFGPGTRLTVT

SEQ ID NO: 714 - protein, 14 aa, Homo sapiens
CASSTTGTLYEQYF

SEQ ID NO: 715 - DNA, 349 bp, Homo sapiens
  source 1..349: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaactggggggtattcaggaggaggtgctgacggactcacctttggcaaagggactcatctaatcatccagccct

SEQ ID NO: 716 - protein, 116 aa, Homo sapiens
GENVEQHPSTLSVQEGDSAVIKCTYSDSASNYFPWYKQELGKRPQLIIDIRSNVGEKKDQRIAVTLNKTAKHFSLHITETQPEDSAVYFCAATGGYSGGGADGLTFGKGTHLIIQP

SEQ ID NO: 717 - protein, 17 aa, Homo sapiens
CAATGGYSGGGADGLTF

SEQ ID NO: 718 - DNA, 346 bp, Homo sapiens
  source 1..346: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagctctaccgggacagcctcctacaatgagcagttcttcgggccagggacacggctcaccgtgctag

SEQ ID NO: 719 - protein, 115 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSSTGTASYNEQFFGPGTRLTVL

SEQ ID NO: 720 - protein, 16 aa, Homo sapiens
CASSSTGTASYNEQFF

SEQ ID NO: 721 - DNA, 340 bp, Homo sapiens
  source 1..340: mol_type = other DNA
ggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgagtgatcggagaggaagctacatacctacatttggaagaggaaccagccttattgttcatccgt

SEQ ID NO: 722 - protein, 113 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSDRRGSYIPTFGRGTSLIVHP

SEQ ID NO: 723 - protein, 14 aa, Homo sapiens
CALSDRRGSYIPTF

SEQ ID NO: 724 - protein, 26 aa, Homo sapiens
GNSVTQMEGPVTLSEEAFLTINCTYT

SEQ ID NO: 725 - DNA, 346 bp, Homo sapiens
  source 1..346: mol_type = other DNA
gagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagccctccgggcgggaggaccacagatacgcagtattttggcccaggcacccggctgacagtgctcg

SEQ ID NO: 726 - protein, 115 aa, Homo sapiens
ETGVTQSPTHLIKTRGQQVTLRCSSQSGHNTVSWYQQALGQGPQFIFQYYREEENGRGNFPPRFSGLQFPNYSSELNVNALELDDSALYLCASSPPGGRTTDTQYFGPGTRLTVL

SEQ ID NO: 727 - protein, 16 aa, Homo sapiens
CASSPPGGRTTDTQYF

SEQ ID NO: 728 - DNA, 346 bp, Homo sapiens
  source 1..346: mol_type = other DNA
aaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccgcactcacgggaggaggaaacaaactcacctttgggacaggcactcagctaaaagtggaactca

SEQ ID NO: 729 - protein, 115 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVRPALTGGGNKLTFGTGTQLKVEL

SEQ ID NO: 730 - protein, 16 aa, Homo sapiens
CAVRPALTGGGNKLTF

SEQ ID NO: 731 - DNA, 334 bp, Homo sapiens
  source 1..334: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagccccacaaagattactttctttggacaaggcaccagactcacagttgtag

SEQ ID NO: 732 - protein, 111 aa, Homo sapiens
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSPTKITFFGQGTRLTVV

SEQ ID NO: 733 - protein, 11 aa, Homo sapiens
CASSPTKITFF

SEQ ID NO: 734 - DNA, 337 bp, Homo sapiens
  source 1..337: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacccggattcaggaaacacacctcttgtctttggaaagggcacaagactttctgtgattgcaa

SEQ ID NO: 735 - protein, 112 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDPDSGNTPLVFGKGTRLSVIA

SEQ ID NO: 736 - protein, 14 aa, Homo sapiens
CATDPDSGNTPLVF

SEQ ID NO: 737 - DNA, 340 bp, Homo sapiens
  source 1..340: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcaggccgggactcttatacgagcagtacttcgggccgggcaccaggctcacggtcacag

SEQ ID NO: 738 - protein, 113 aa, Homo sapiens
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASRPGLLYEQYFGPGTRLTVT

SEQ ID NO: 739 - protein, 13 aa, Homo sapiens
CASRPGLLYEQYF

SEQ ID NO: 740 - DNA, 334 bp, Homo sapiens
  source 1..334: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtatgggaaacacacctcttgtctttggaaagggcacaagactttctgtgattgcaa

SEQ ID NO: 741 - protein, 111 aa, Homo sapiens
GENVEQHPSTLSVQEGDSAVIKCTYSDSASNYFPWYKQELGKGPQLIIDIRSNVGEKKDQRIAVTLNKTAKHFSLHITETQPEDSAVYFCAASMGNTPLVFGKGTRLSVIA

SEQ ID NO: 742 - protein, 12 aa, Homo sapiens
CAASMGNTPLVF

SEQ ID NO: 743 - DNA, 340 bp, Homo sapiens
  source 1..340: mol_type = other DNA
ggagctggagtctcccagtcccccagtaacaaggtcacagagaagggaaaggatgtagagctcaggtgtgatccaatttcaggtcatactgccctttactggtaccgacagaggctggggcagggcctggagtttttaatttacttccaaggcaacagtgcaccagacaaatcagggctgcccagtgatcgcttctctgcagagaggactggggaatccgtctccactctgacgatccagcgcacacagcaggaggactcggccgtgtatctctgtgccagcagcccaactagcgggtacgagcagtacttcgggccgggcaccaggctcacggtcacag

SEQ ID NO: 744 - protein, 113 aa, Homo sapiens
GAGVSQSPSNKVTEKGKDVELRCDPISGHTALYWYRQRLGQGLEFLIYFQGNSAPDKSGLPSDRFSAERTGESVSTLTIQRTQQEDSAVYLCASSPTSGYEQYFGPGTRLTVT

SEQ ID NO: 745 - protein, 13 aa, Homo sapiens
CASSPTSGYEQYF

SEQ ID NO: 746 - protein, 17 aa, Homo sapiens
LYWYRQRLGQGLEFLIY

SEQ ID NO: 747 - protein, 37 aa, Homo sapiens
PDKSGLPSDRFSAERTGESVSTLTIQRTQQEDSAVYL

SEQ ID NO: 748 - DNA, 343 bp, Homo sapiens
  source 1..343: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagctcctattataaccagggaggaaagcttatcttcggacagggaacggagttatctgtgaaaccca

SEQ ID NO: 749 - protein, 114 aa, Homo sapiens
GENVEQHPSTLSVQEGDSAVIKCTYSDSASNYFPWYKQELGKGPQLIIDIRSNVGEKKDQRIAVTLNKTAKHFSLHITETQPEDSAVYFCAASSYYNQGGKLIFGQGTELSVKP

SEQ ID NO: 750 - protein, 15 aa, Homo sapiens
CAASSYYNQGGKLIF

SEQ ID NO: 751 - DNA, 343 bp, Homo sapiens
  source 1..343: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtgggcggacagggcaatcagccccagcattttggtgatgggactcgactctccatcctag

SEQ ID NO: 752 - protein, 114 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSVGGQGNQPQHFGDGTRLSIL

SEQ ID NO: 753 - protein, 15 aa, Homo sapiens
CASSVGGQGNQPQHF

SEQ ID NO: 754 - DNA, 331 bp, Homo sapiens
  source 1..331: mol_type = other DNA
ggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctacatatgctggcaacaaccgtaagctgatttggggattgggaacaagcctggcagtaaatccga

SEQ ID NO: 755 - protein, 110 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCATYAGNNRKLIWGLGTSLAVNP

SEQ ID NO: 756 - protein, 13 aa, Homo sapiens
CATYAGNNRKLIW

SEQ ID NO: 757 - DNA, 343 bp, Homo sapiens
  source 1..343: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcccattggagtcgaacaccggggagctgttttttggagaaggctctaggctgaccgtactgg

SEQ ID NO: 758 - protein, 114 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSPLESNTGELFFGEGSRLTVL

SEQ ID NO: 759 - protein, 15 aa, Homo sapiens
CASSPLESNTGELFF

SEQ ID NO: 760 - DNA, 334 bp, Homo sapiens
  source 1..334: mol_type = other DNA
atactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccctccttaacaccggtaaccagttctattttgggacagggacaagtttgacggtcattccaa

SEQ ID NO: 761 - protein, 111 aa, Homo sapiens
ILNVEQSPQSLHVQEGDSTNFTCSFPSSNFYALHWYRWETAKSPEALFVMTLNGDEKKKGRISATLNTKEGYSYLYIKGSQPEDSATYLCALLNTGNQFYFGTGTSLTVIP

SEQ ID NO: 762 - protein, 6 aa, Homo sapiens
SSNFYA

SEQ ID NO: 763 - protein, 7 aa, Homo sapiens
MTLNGDE

SEQ ID NO: 764 - protein, 12 aa, Homo sapiens
CALLNTGNQFYF

SEQ ID NO: 765 - protein, 26 aa, Homo sapiens
ILNVEQSPQSLHVQEGDSTNFTCSFP

SEQ ID NO: 766 - protein, 17 aa, Homo sapiens
LHWYRWETAKSPEALFV

SEQ ID NO: 767 - protein, 33 aa, Homo sapiens
KKKGRISATLNTKEGYSYLYIKGSQPEDSATYL

SEQ ID NO: 768 - DNA, 346 bp, Homo sapiens
  source 1..346: mol_type = other DNA
gaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagactcggccctgtatctctgtgccagcagcccagggacagggggcgcggccggggagctgttttttggagaaggctctaggctgaccgtactgg

SEQ ID NO: 769 - protein, 115 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSPGTGGAAGELFFGEGSRLTVL

SEQ ID NO: 770 - protein, 16 aa, Homo sapiens
CASSPGTGGAAGELFF

SEQ ID NO: 771 - DNA, 340 bp, Homo sapiens
  source 1..340: mol_type = other DNA
ggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcggctataggattttctggtggctacaataagctgatttttggagcagggaccaggctggctgtacacccat

SEQ ID NO: 772 - protein, 113 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAAIGFSGGYNKLIFGAGTRLAVHP

SEQ ID NO: 773 - protein, 15 aa, Homo sapiens
CAAIGFSGGYNKLIF

SEQ ID NO: 774 - protein, 10 aa, Homo sapiens
GAGTRLAVHP

SEQ ID NO: 775 - DNA, 340 bp, Homo sapiens
  source 1..340: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtattgcaggggcggactacgagcagtacttcgggccgggcaccaggctcacggtcacag

SEQ ID NO: 776 - protein, 113 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIAGADYEQYFGPGTRLTVT

SEQ ID NO: 777 - protein, 14 aa, Homo sapiens
CASSIAGADYEQYF

SEQ ID NO: 778 - DNA, 349 bp, Homo sapiens
  source 1..349: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcacagccaccaacgccaacgaacaccggtaaccagttctattttgggacagggacaagtttgacggtcattccaa

SEQ ID NO: 779 - protein, 116 aa, Homo sapiens
GENVEQHPSTLSVQEGDSAVIKCTYSDSASNYFPWYKQELGKRPQLIIDIRSNVGEKKDQRIAVTLNKTAKHFSLHITETQPEDSAVYFCAAQPPTPTNTGNQFYFGTGTSLTVIP

SEQ ID NO: 780 - protein, 17 aa, Homo sapiens
CAAQPPTPTNTGNQFYF

SEQ ID NO: 781 - DNA, 340 bp, Homo sapiens
  source 1..340: mol_type = other DNA
gctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagaccacagggaactaatgaaaaactgttttttggcagtggaacccagctctctgtcttgg

SEQ ID NO: 782 - protein, 113 aa, Homo sapiens
AAGVIQSPRHLIKEKRETATLKCYPIPRHDTVYWYQQGPGQDPQFLISFYEKMQSDKGSIPDRFSAQQFSDYHSELNMSSLELGDSALYFCASRPQGTNEKLFFGSGTQLSVL

SEQ ID NO: 783 - protein, 14 aa, Homo sapiens
CASRPQGTNEKLFF

SEQ ID NO: 784 - DNA, 334 bp, Homo sapiens
  source 1..334: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtggaaaccagtggctctaggttgacctttggggaaggaacacagctcacagtgaatcctg

SEQ ID NO: 785 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVETSGSRLTFGEGTQLTVNP

SEQ ID NO: 786 - protein, 12 aa, Homo sapiens
CAVETSGSRLTF

SEQ ID NO: 787 - protein, 10 aa, Homo sapiens
GEGTQLTVNP

SEQ ID NO: 788 - DNA, 349 bp, Homo sapiens
  source 1..349: mol_type = other DNA
ggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcggagctagcgggggggctcacaccggggagctgttttttggagaaggctctaggctgaccgtactgg

SEQ ID NO: 789 - protein, 116 aa, Homo sapiens
GAGVSQSPRYKVAKRGQDVALRCDPISGHVSLFWYQQALGQGPEFLTYFQNEAQLDKSGLPSDRFFAERPEGSVSTLKIQRTQQEDSAVYLCASGASGGAHTGELFFGEGSRLTVL

SEQ ID NO: 790 - protein, 16 aa, Homo sapiens
CASGASGGAHTGELFF

SEQ ID NO: 791 - protein, 26 aa, Homo sapiens
GAGVSQSPRYKVAKRGQDVALRCDPI

SEQ ID NO: 792 - protein, 17 aa, Homo sapiens
LFWYQQALGQGPEFLTY

SEQ ID NO: 793 - protein, 11 aa, Homo sapiens
CAESEGGKLIF

SEQ ID NO: 794 - DNA, 328 bp, Homo sapiens
  source 1..328: mol_type = other DNA
ggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagagcgagggaggaaagcttatcttcggacagggaacggagttatctgtgaaaccca

SEQ ID NO: 795 - protein, 109 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAESEGGKLIFGQGTELSVKP

SEQ ID NO: 796 - protein, 11 aa, Homo sapiens
CAESEGGKLIF

SEQ ID NO: 797 - DNA, 340 bp, Homo sapiens
  source 1..340: mol_type = other DNA
gatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagacctttgaggagcaatcagccccagcattttggtgatgggactcgactctccatcctag

SEQ ID NO: 798 - protein, 113 aa, Homo sapiens
DVKVTQSSRYLVKRTGEKVFLECVQDMDHENMFWYRQDPGLGLRLIYFSYDVKMKEKGDIPEGYSVSREKKERFSLILESASTNQTSMYLCASRPLRSNQPQHFGDGTRLSIL

SEQ ID NO: 799 - protein, 14 aa, Homo sapiens
CASRPLRSNQPQHF

SEQ ID NO: 800 - DNA, 337 bp, Homo sapiens
  source 1..337: mol_type = other DNA
ggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagaagctaataatgcaggcaacatgctcacctttggagggggaacaaggttaatggtcaaacccc

SEQ ID NO: 801 - protein, 112 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVREANNAGNMLTFGGGTRLMVKP

SEQ ID NO: 802 - protein, 15 aa, Homo sapiens
CAVREANNAGNMLTF

SEQ ID NO: 803 - DNA, 331 bp, Homo sapiens
  source 1..331: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgagtatgagcagttcttcgggccagggacacggctcaccgtgctag

SEQ ID NO: 804 - protein, 110 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVEYEQFFGPGTRLTVL

SEQ ID NO: 805 - protein, 9 aa, Homo sapiens
CSVEYEQFF

SEQ ID NO: 806 - DNA, 343 bp, Homo sapiens
  source 1..343: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaactcgattgaaaactttggaaatgagaaattaacctttgggactggaacaagactcaccatcataccca

SEQ ID NO: 807 - protein, 114 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVNSIENFGNEKLTFGTGTRLTIIP

SEQ ID NO: 808 - protein, 16 aa, Homo sapiens
CAVNSIENFGNEKLTF

SEQ ID NO: 809 - DNA, 352 bp, Homo sapiens
  source 1..352: mol_type = other DNA
gctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggatctcctggtcaaattccgggtatgcactcaacttcggcaaaggcacctcgctgttggtcacacccc

SEQ ID NO: 810 - protein, 117 aa, Homo sapiens
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRISWSNSGYALNFGKGTSLLVTP

SEQ ID NO: 811 - protein, 16 aa, Homo sapiens
CAYRISWSNSGYALNF

SEQ ID NO: 812 - protein, 10 aa, Homo sapiens
GKGTSLLVTP

SEQ ID NO: 813 - DNA, 346 bp, Homo sapiens
  source 1..346: mol_type = other DNA
aatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcacaagacagggccggggccaacgtcctgactttcggggccggcagcaggctgaccgtgctgg

SEQ ID NO: 814 - protein, 115 aa, Homo sapiens
NAGVMQNPRHLVRRRGQEARLRCSPMKGHSHVYWYRQLPEEGLKFMVYLQKENIIDESGMPKERFSAEFPKEGPSILRIQQVVRGDSAAYFCASSQDRAGANVLTFGAGSRLTVL

SEQ ID NO: 815 - protein, 15 aa, Homo sapiens
CASSQDRAGANVLTF

SEQ ID NO: 816 - protein, 9 aa, Homo sapiens
GAGSRLTVL

SEQ ID NO: 817 - DNA, 328 bp, Homo sapiens
  source 1..328: mol_type = other DNA
gatgctaagaccacccagcccccctccatggattgcgctgaaggaagagctgcaaacctgccttgtaatcactctaccatcagtggaaatgagtatgtgtattggtatcgacagattcactcccaggggccacagtatatcattcatggtctaaaaaacaatgaaaccaatgaaatggcctctctgatcatcacagaagacagaaagtccagcaccttgatcctgccccacgctacgctgagagacactgctgtgtactattgcatcgtccgaggaggtgctgacggactcacctttggcaaagggactcatctaatcatccagccct

SEQ ID NO: 818 - protein, 109 aa, Homo sapiens
DAKTTQPPSMDCAEGRAANLPCNHSTISGNEYVYWYRQIHSQGPQYIIHGLKNNETNEMASLIITEDRKSSTLILPHATLRDTAVYYCIVRGGADGLTFGKGTHLIIQP

SEQ ID NO: 819 - protein, 12 aa, Homo sapiens
CIVRGGADGLTF

SEQ ID NO: 820 - DNA, 343 bp, Homo sapiens
  source 1..343: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcaatcggcaacgagataattcacccctccactttgggaatgggaccaggctcactgtgacag

SEQ ID NO: 821 - protein, 114 aa, Homo sapiens
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASNRQRDNSPLHFGNGTRLTVT

SEQ ID NO: 822 - protein, 14 aa, Homo sapiens
CASNRQRDNSPLHF

SEQ ID NO: 823 - DNA, 349 bp, Homo sapiens
  source 1..349: mol_type = other DNA
gcccagaagataactcaaacccaaccaggaatgttcgtgcaggaaaaggaggctgtgactctggactgcacatatgacaccagtgatcaaagttatggtctattctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgagagagtggttttcagatggccagaagctgctctttgcaagggggaccatgttaaaggtggatctta

SEQ ID NO: 824 - protein, 116 aa, Homo sapiens
AQKITQTQPGMFVQEKEAVTLDCTYDTSDQSYGLFWYKQPSSGEMIFLIYQGSYDEQNATEGRYSLNFQKARKSANLVISASQLGDSAMYFCAMREWFSDGQKLLFARGTMLKVDL

SEQ ID NO: 825 - protein, 15 aa, Homo sapiens
CAMREWFSDGQKLLF

SEQ ID NO: 826 - DNA, 340 bp, Homo sapiens
  source 1..340: mol_type = other DNA
gatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatggtcaagggaagcagccccagcattttggtgatgggactcgactctccatcctag

SEQ ID NO: 827 - protein, 113 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFCATSDGQGKQPQHFGDGTRLSIL

SEQ ID NO: 828 - protein, 14 aa, Homo sapiens
CATSDGQGKQPQHF

SEQ ID NO: 829 - DNA, 337 bp, Homo sapiens
  source 1..337: mol_type = other DNA
agccaaaagatagaacagaattccgaggccctgaacattcaggagggtaaaacggccaccctgacctgcaactatacaaactattccccagcatacttacagtggtaccgacaagatccaggaagaggccctgttttcttgctactcatacgtgaaaatgagaaagaaaaaaggaaagaaagactgaaggtcacctttgataccacccttaaacagagtttgtttcatatcacagcctcccagcctgcagactcagctacctacctctgtgctctagagggtaatgcaggcaacatgctcacctttggagggggaacaaggttaatggtcaaacccc

SEQ ID NO: 830 - protein, 112 aa, Homo sapiens
SQKIEQNSEALNIQEGKTATLTCNYTNYSPAYLQWYRQDPGRGPVFLLLIRENEKEKRKERLKVTFDTTLKQSLFHITASQPADSATYLCALEGNAGNMLTFGGGTRLMVKP

SEQ ID NO: 831 - protein, 6 aa, Homo sapiens
NYSPAY

SEQ ID NO: 832 - protein, 7 aa, Homo sapiens
IRENEKE

SEQ ID NO: 833 - protein, 13 aa, Homo sapiens
CALEGNAGNMLTF

SEQ ID NO: 834 - protein, 26 aa, Homo sapiens
SQKIEQNSEALNIQEGKTATLTCNYT

SEQ ID NO: 835 - protein, 17 aa, Homo sapiens
LQWYRQDPGRGPVFLLL

SEQ ID NO: 836 - protein, 33 aa, Homo sapiens
KRKERLKVTFDTTLKQSLFHITASQPADSATYL

SEQ ID NO: 837 - DNA, 346 bp, Homo sapiens
  source 1..346: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtagggggggcgcgacagcctcgtggctacaccttcggttcggggaccaggttaaccgttgtag

SEQ ID NO: 838 - protein, 115 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSVGGARQPRGYTFGSGTRLTVV

SEQ ID NO: 839 - protein, 16 aa, Homo sapiens
CASSVGGARQPRGYTF

SEQ ID NO: 840 - DNA, 334 bp, Homo sapiens
  source 1..334: mol_type = other DNA
atactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccttaatttcaggaaacacacctcttgtctttggaaagggcacaagactttctgtgattgcaa

SEQ ID NO: 841 - protein, 111 aa, Homo sapiens
ILNVEQSPQSLHVQEGDSTNFTCSFPSSNFYALHWYRWETAKSPEALFVMTLNGDEKKKGRISATLNTKEGYSYLYIKGSQPEDSATYLCALISGNTPLVFGKGTRLSVIA

SEQ ID NO: 842 - protein, 12 aa, Homo sapiens
CALISGNTPLVF

SEQ ID NO: 843 - DNA, 352 bp, Homo sapiens
  source 1..352: mol_type = other DNA
gaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgccagcagccagaccgggacagggggcacgttcaccggggagctgttttttggagaaggctctaggctgaccgtactgg

SEQ ID NO: 844 - protein, 117 aa, Homo sapiens
EAGVTQSPTHLIKTRGQQATLRCSPISGHTSVYWYQQALGLGLQFLLWYDEGEERNRGNFPPRFSGRQFPNYSSELNVNALELEDSALYLCASSQTGTGGTFTGELFFGEGSRLTVL

SEQ ID NO: 845 - protein, 5 aa, Homo sapiens
SGHTS

SEQ ID NO: 846 - protein, 6 aa, Homo sapiens
YDEGEE

SEQ ID NO: 847 - protein, 18 aa, Homo sapiens
CASSQTGTGGTFTGELFF

SEQ ID NO: 848 - protein, 26 aa, Homo sapiens
EAGVTQSPTHLIKTRGQQATLRCSPI

SEQ ID NO: 849 - protein, 17 aa, Homo sapiens
VYWYQQALGLGLQFLLW

SEQ ID NO: 850 - protein, 36 aa, Homo sapiens
RNRGNFPPRFSGRQFPNYSSELNVNALELEDSALYL

SEQ ID NO: 851 - DNA, 331 bp, Homo sapiens
  source 1..331: mol_type = other DNA
caacaaccagtgcagagtcctcaagccgtgatcctccgagaaggggaagatgctgtcatcaactgcagttcctccaaggctttatattctgtacactggtacaggcagaagcatggtgaagcacccgtcttcctgatgatattactgaagggtggagaacagaagggtcatgaaaaaatatctgcttcatttaatgaaaaaaagcagcaaagctccctgtaccttacggcctcccagctcagttactcaggaacctacttctgcggcacacccagatttaacaccgacaagctcatctttgggactgggaccagattacaagtctttccaa

SEQ ID NO: 852 - protein, 110 aa, Homo sapiens
QQPVQSPQAVILREGEDAVINCSSSKALYSVHWYRQKHGEAPVFLMILLKGGEQKGHEKISASFNEKKQQSSLYLTASQLSYSGTYFCGTPRFNTDKLIFGTGTRLQVFP

SEQ ID NO: 853 - protein, 13 aa, Homo sapiens
CGTPRFNTDKLIF

SEQ ID NO: 854 - protein, 17 aa, Homo sapiens
VHWYRQKHGEAPVFLMI

SEQ ID NO: 855 - protein, 33 aa, Homo sapiens
KGHEKISASFNEKKQQSSLYLTASQLSYSGTYF

SEQ ID NO: 856 - DNA, 340 bp, Homo sapiens
  source 1..340: mol_type = other DNA
gaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtggttcctatagcaatcagccccagcattttggtgatgggactcgactctccatcctag

SEQ ID NO: 857 - protein, 113 aa, Homo sapiens
EADIYQTPRYLVIGTGKKITLECSQTMGHDKMYWYQQDPGMELHLIHYSYGVNSTEKGDLSSESTVSRIRTEHFPLTLESARPSHTSQYLCASSGSYSNQPQHFGDGTRLSIL

SEQ ID NO: 858 - protein, 5 aa, Homo sapiens
MGHDK

SEQ ID NO: 859 - protein, 6 aa, Homo sapiens
SYGVNS

SEQ ID NO: 860 - protein, 14 aa, Homo sapiens
CASSGSYSNQPQHF

SEQ ID NO: 861 - protein, 26 aa, Homo sapiens
EADIYQTPRYLVIGTGKKITLECSQT

SEQ ID NO: 862 - protein, 17 aa, Homo sapiens
MYWYQQDPGMELHLIHY

SEQ ID NO: 863 - protein, 36 aa, Homo sapiens
TEKGDLSSESTVSRIRTEHFPLTLESARPSHTSQYL

SEQ ID NO: 864 - DNA, 334 bp, Homo sapiens
  source 1..334: mol_type = other DNA
cagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatcaaaattcagggagcccagaagctggtatttggccaaggaaccaggctgactatcaacccaa

SEQ ID NO: 865 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAIKIQGAQKLVFGQGTRLTINP

SEQ ID NO: 866 - protein, 13 aa, Homo sapiens
CAIKIQGAQKLVF

SEQ ID NO: 867 - protein, 10 aa, Homo sapiens
GQGTRLTINP

SEQ ID NO: 868 - DNA, 343 bp, Homo sapiens
  source 1..343: mol_type = other DNA
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaagaagacaggtctggaaacaccatatattttggagagggaagttggctcactgttgtag

SEQ ID NO: 869 - protein, 114 aa, Homo sapiens
DTAVSQTPKYLVTQMGNDKSIKCEQNLGHDTMYWYKQDSKKFLKIMFSYNNKELIINETVPNRFSPKSPDKAHLNLHINSLELGDSAVYFCASSQEDRSGNTIYFGEGSWLTVV

SEQ ID NO: 870 - protein, 15 aa, Homo sapiens
CASSQEDRSGNTIYF

SEQ ID NO: 871 - protein, 9 aa, Homo sapiens
GEGSWLTVV

SEQ ID NO: 872 - DNA, 337 bp, Homo sapiens
  source 1..337: mol_type = other DNA
ggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagatccttttaaggggaggcttcaaaactatctttggagcaggaacaagactatttgttaaagcaa

SEQ ID NO: 873 - protein, 112 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAEILLRGGFKTIFGAGTRLFVKA

SEQ ID NO: 874 - protein, 14 aa, Homo sapiens
CAEILLRGGFKTIF

SEQ ID NO: 875 - DNA, 340 bp, Homo sapiens
  source 1..340: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtatcatctcaggttcctacgagcagtacttcgggccgggcaccaggctcacggtcacag

SEQ ID NO: 876 - protein, 113 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSIISGSYEQYFGPGTRLTVT

SEQ ID NO: 877 - protein, 14 aa, Homo sapiens
CASSIISGSYEQYF

SEQ ID NO: 878 - DNA, 337 bp, Homo sapiens
  source 1..337: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgcggtgagcaactataaactgacatttggaaaaggaactctcttaaccgtgaatccaa

SEQ ID NO: 879 - protein, 112 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAASAVSNYKLTFGKGTLLTVNP

SEQ ID NO: 880 - protein, 13 aa, Homo sapiens
CAASAVSNYKLTF

SEQ ID NO: 881 - DNA, 343 bp, Homo sapiens
  source 1..343: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagcgggagggccacgtcctacaatgagcagttcttcgggccagggacacggctcaccgtgctag

SEQ ID NO: 882 - protein, 114 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSGRATSYNEQFFGPGTRLTVL

SEQ ID NO: 883 - protein, 15 aa, Homo sapiens
CASSGRATSYNEQFF

SEQ ID NO: 884 - DNA, 343 bp, Homo sapiens
  source 1..343: mol_type = other DNA
gcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggggacggtggtgctacaaacaagctcatctttggaactggcactctgcttgctgtccagccaa

SEQ ID NO: 885 - protein, 114 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGDGGATNKLIFGTGTLLAVQP

SEQ ID NO: 886 - protein, 6 aa, Homo sapiens
YGATPY

SEQ ID NO: 887 - protein, 8 aa, Homo sapiens
YFSGDTLV

SEQ ID NO: 888 - protein, 14 aa, Homo sapiens
CAVGDGGATNKLIF

SEQ ID NO: 889 - protein, 26 aa, Homo sapiens
AQSVTQPDIHITVSEGASLELRCNYS

SEQ ID NO: 890 - protein, 17 aa, Homo sapiens
LFWYVQSPGQGLQLLLK

SEQ ID NO: 891 - protein, 33 aa, Homo sapiens
QGIKGFEAEFKRSQSSFNLRKPSVHWSDAAEYF

SEQ ID NO: 892 - DNA, 340 bp, Homo sapiens
  source 1..340: mol_type = other DNA
gaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagccccctcgatccctacgagcagtacttcgggccgggcaccaggctcacggtcacag

SEQ ID NO: 893 - protein, 113 aa, Homo sapiens
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSPLDPYEQYFGPGTRLTVT

SEQ ID NO: 894 - protein, 5 aa, Homo sapiens
SGHAT

SEQ ID NO: 895 - protein, 6 aa, Homo sapiens
FQNNGV

SEQ ID NO: 896 - protein, 13 aa, Homo sapiens
CASSPLDPYEQYF

SEQ ID NO: 897 - protein, 26 aa, Homo sapiens
EAGVAQSPRYKIIEKRQSVAFWCNPI

SEQ ID NO: 898 - protein, 17 aa, Homo sapiens
LYWYQQILGQGPKLLIQ

SEQ ID NO: 899 - protein, 37 aa, Homo sapiens
VDDSQLPKDRFSAERLKGVDSTLKIQPAKLEDSAVYL

SEQ ID NO: 900 - DNA, 343 bp, Homo sapiens
  source 1..343: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcagggtctaaatccgggggttaccagaaagttacctttggaactggaacaaagctccaagtcatcccaa

SEQ ID NO: 901 - protein, 114 aa, Homo sapiens
GENVEQHPSTLSVQEGDSAVIKCTYSDSASNYFPWYKQELGKRPQLIIDIRSNVGEKKDQRIAVTLNKTAKHFSLHITETQPEDSAVYFCAAGSKSGGYQKVTFGTGTKLQVIP

SEQ ID NO: 902 - protein, 15 aa, Homo sapiens
CAAGSKSGGYQKVTF

SEQ ID NO: 903 - protein, 10 aa, Homo sapiens
GTGTKLQVIP

SEQ ID NO: 904 - DNA, 349 bp, Homo sapiens
  source 1..349: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtacccctagggctagcggggagatcgccgatgagcagttcttcgggccagggacacggctcaccgtgctag

SEQ ID NO: 905 - protein, 116 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASTPRASGEIADEQFFGPGTRLTVL

SEQ ID NO: 906 - protein, 17 aa, Homo sapiens
CASTPRASGEIADEQFF

SEQ ID NO: 907 - DNA, 349 bp, Homo sapiens
  source 1..349: mol_type = other DNA
gctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggggggatacaacttcaacaaattttactttggatctgggaccaaactcaatgtaaaaccaa

SEQ ID NO: 908 - protein, 116 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEGGYNFNKFYFGSGTKLNVKP

SEQ ID NO: 909 - protein, 15 aa, Homo sapiens
CALSEGGYNFNKFYF

SEQ ID NO: 910 - protein, 10 aa, Homo sapiens
GSGTKLNVKP

SEQ ID NO: 911 - DNA, 349 bp, Homo sapiens
  source 1..349: mol_type = other DNA
gaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttatttctgtgccagcagccaagaagtcagccgcccacacaatgagcagttcttcgggccagggacacggctcaccgtgctag

SEQ ID NO: 912 - protein, 116 aa, Homo sapiens
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSQEVSRPHNEQFFGPGTRLTVL

SEQ ID NO: 913 - protein, 16 aa, Homo sapiens
CASSQEVSRPHNEQFF

SEQ ID NO: 914 - DNA, 334 bp, Homo sapiens
  source 1..334: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggagcctctcacgggaggaggaaacaaactcacctttgggacaggcactcagctaaaagtggaactca

SEQ ID NO: 915 - protein, 111 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVEPLTGGGNKLTFGTGTQLKVEL

SEQ ID NO: 916 - protein, 15 aa, Homo sapiens
CAVEPLTGGGNKLTF

SEQ ID NO: 917 - DNA, 346 bp, Homo sapiens
  source 1..346: mol_type = other DNA
gatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtttattacccagagggggtggtggtggctacaccttcggttcggggaccaggttaaccgttgtag

SEQ ID NO: 918 - protein, 115 aa, Homo sapiens
DVKVTQSSRYLVKRTGEKVFLECVQDMDHENMFWYRQDPGLGLRLIYFSYDVKMKEKGDIPEGYSVSREKKERFSLILESASTNQTSMYLCASSLLPRGGGGGYTFGSGTRLTVV

SEQ ID NO: 919 - protein, 16 aa, Homo sapiens
CASSLLPRGGGGGYTF

SEQ ID NO: 920 - DNA, 319 bp, Homo sapiens
  source 1..319: mol_type = other DNA
aaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgttgcagattcaggaaacacacctcttgtctttggaaagggcacaagactttctgtgattgcaa

SEQ ID NO: 921 - protein, 106 aa, Homo sapiens
KDQVFQPSTVASSEGAVVEIFCNHSVSNAYNFFWYLHFPGCAPRLLVKGSKPSQQGRYNMTYERFSSSLLILQVREADAAVYYCAVADSGNTPLVFGKGTRLSVIA

SEQ ID NO: 922 - protein, 13 aa, Homo sapiens
CAVADSGNTPLVF

SEQ ID NO: 923 - DNA, 337 bp, Homo sapiens
  source 1..337: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgctagcctcacggaccagttcttcgggccagggacacggctcaccgtgctag

SEQ ID NO: 924 - protein, 112 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYASLTDQFFGPGTRLTVL

SEQ ID NO: 925 - protein, 13 aa, Homo sapiens
CASSYASLTDQFF

SEQ ID NO: 926 - DNA, 328 bp, Homo sapiens
  source 1..328: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggagctgaggatgccagactcatgtttggagatggaactcagctggtggtgaagccca

SEQ ID NO: 927 - protein, 109 aa, Homo sapiens
TQLLEQSPQFLSIQEGENLTVYCNSSSVFSSLQWYRQEPGEGPVLLVTVVTGGEVKKLKRLTFQFGDARKDSSLHITAAQPGDTGLYLCAGAEDARLMFGDGTQLVVKP

SEQ ID NO: 928 - protein, 11 aa, Homo sapiens
CAGAEDARLMF

SEQ ID NO: 929 - DNA, 352 bp, Homo sapiens
  source 1..352: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttgagtcggggacagggggcttttcagccccagcattttggtgatgggactcgactctccatcctag

SEQ ID NO: 930 - protein, 117 aa, Homo sapiens
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLSRGQGAFQPQHFGDGTRLSIL

SEQ ID NO: 931 - protein, 17 aa, Homo sapiens
CASSLSRGQGAFQPQHF

SEQ ID NO: 932 - DNA, 337 bp, Homo sapiens
  source 1..337: mol_type = other DNA
gacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaggcaactaatgaaaaactgttttttggcagtggaacccagctctctgtcttgg

SEQ ID NO: 933 - protein, 112 aa, Homo sapiens
DTEVTQTPKHLVMGMTNKKSLKCEQHMGHRAMYWYKQKAKKPPELMFVYSYEKLSINESVPSRFSPECPNSSLLNLHLHALQPEDSALYLCASSQATNEKLFFGSGTQLSVL

SEQ ID NO: 934 - protein, 13 aa, Homo sapiens
CASSQATNEKLFF

SEQ ID NO: 935 - DNA, 337 bp, Homo sapiens
  source 1..337: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcatatggtactagctatggaaagctgacatttggacaagggaccatcttgactgtccatccaa

SEQ ID NO: 936 - protein, 112 aa, Homo sapiens
GENVEQHPSTLSVQEGDSAVIKCTYSDSASNYFPWYKQELGKGPQLIIDIRSNVGEKKDQRIAVTLNKTAKHFSLHITETQPEDSAVYFCAAYGTSYGKLTFGQGTILTVHP

SEQ ID NO: 937 - protein, 13 aa, Homo sapiens
CAAYGTSYGKLTF

SEQ ID NO: 938 - DNA, 349 bp, Homo sapiens
  source 1..349: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgcttgccgtgacaggggctacactgaagctttctttggacaaggcaccagactcacagttgtag

SEQ ID NO: 939 - protein, 116 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSACRDRGYTEAFFGQGTRLTVV

SEQ ID NO: 940 - protein, 14 aa, Homo sapiens
CSACRDRGYTEAFF

SEQ ID NO: 941 - DNA, 340 bp, Homo sapiens
  source 1..340: mol_type = other DNA
gctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggttcaataacaatgacatgcgctttggagcagggaccagactgacagtaaaaccaa

SEQ ID NO: 942 - protein, 113 aa, Homo sapiens
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRFNNNDMRFGAGTRLTVKP

SEQ ID NO: 943 - protein, 12 aa, Homo sapiens
CAYRFNNNDMRF

SEQ ID NO: 944 - DNA, 340 bp, Homo sapiens
  source 1..340: mol_type = other DNA
tctcagactattcatcaatggccagcgaccctggtgcagcctgtgggcagcccgctctctctggagtgcactgtggagggaacatcaaaccccaacctatactggtaccgacaggctgcaggcaggggcctccagctgctcttctactccgttggtattggccagatcagctctgaggtgccccagaatctctcagcctccagaccccaggaccggcagttcatcctgagttctaagaagctccttctcagtgactctggcttctatctctgtgcctggacctccggacccagcacagatacgcagtattttggcccaggcacccggctgacagtgctcg

SEQ ID NO: 945 - protein, 113 aa, Homo sapiens
SQTIHQWPATLVQPVGSPLSLECTVEGTSNPNLYWYRQAAGRGLQLLFYSVGIGQISSEVPQNLSASRPQDRQFILSSKKLLLSDSGFYLCAWTSGPSTDTQYFGPGTRLTVL

SEQ ID NO: 946 - protein, 14 aa, Homo sapiens
CAWTSGPSTDTQYF

SEQ ID NO: 947 - DNA, 334 bp, Homo sapiens
  source 1..334: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcggccccctctcgaggcttcaaaactatctttggagcaggaacaagactatttgttaaagcaa

SEQ ID NO: 948 - protein, 111 aa, Homo sapiens
GENVEQHPSTLSVQEGDSAVIKCTYSDSASNYFPWYKQELGKGPQLIIDIRSNVGEKKDQRIAVTLNKTAKHFSLHITETQPEDSAVYFCAAPSRGFKTIFGAGTRLFVKA

SEQ ID NO: 949 - protein, 12 aa, Homo sapiens
CAAPSRGFKTIF

SEQ ID NO: 950 - DNA, 340 bp, Homo sapiens
  source 1..340: mol_type = other DNA
ggtgctggagtctcccagtctcccaggtacaaagtcacaaagaggggacaggatgtagctctcaggtgtgatccaatttcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgctgtgccagcagctcaggactcacagatacgcagtattttggcccaggcacccggctgacagtgctcg

SEQ ID NO: 951 - protein, 113 aa, Homo sapiens
GAGVSQSPRYKVTKRGQDVALRCDPISGHVSLYWYRQALGQGPEFLTYFNYEAQQDKSGLPNDRFSAERPEGSISTLTIQRTEQRDSAMYRCASSSGLTDTQYFGPGTRLTVL

SEQ ID NO: 952 - protein, 13 aa, Homo sapiens
CASSSGLTDTQYF

SEQ ID NO: 953 - DNA, 337 bp, Homo sapiens
  source 1..337: mol_type = other DNA
aaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgccctagataactatggtcagaattttgtctttggtcccggaaccagattgtccgtgctgccct

SEQ ID NO: 954 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAALDNYGQNFVFGPGTRLSVLP

SEQ ID NO: 955 - protein, 13 aa, Homo sapiens
CAALDNYGQNFVF

SEQ ID NO: 956 - DNA, 343 bp, Homo sapiens
  source 1..343: mol_type = other DNA
gacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaaggggcgggggggtacaatgagcagttcttcgggccagggacacggctcaccgtgctag

SEQ ID NO: 957 - protein, 114 aa, Homo sapiens
DTEVTQTPKHLVMGMTNKKSLKCEQHMGHRAMYWYKQKAKKPPELMFVYSYEKLSINESVPSRFSPECPNSSLLNLHLHALQPEDSALYLCASSQGAGGYNEQFFGPGTRLTVL

SEQ ID NO: 958 - protein, 15 aa, Homo sapiens
CASSQGAGGYNEQFF

SEQ ID NO: 959 - DNA, 328 bp, Homo sapiens
  source 1..328: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtatagccaagctcatctttgggactgggaccagattacaagtctttccaa

SEQ ID NO: 960 - protein, 109 aa, Homo sapiens
GENVEQHPSTLSVQEGDSAVIKCTYSDSASNYFPWYKQELGKRPQLIIDIRSNVGEKKDQRIAVTLNKTAKHFSLHITETQPEDSAVYFCAASIAKLIFGTGTRLQVFP

SEQ ID NO: 961 - protein, 10 aa, Homo sapiens
CAASIAKLIF

SEQ ID NO: 962 - DNA, 343 bp, Homo sapiens
  source 1..343: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcactattataatggagaagagagagcaaaaggaaacattcttgaacgattctccgcacaacagttccctgacttgcactctgaactaaacctgagctctctggagctgggggactcagctttgtatttctgtgccagcagcttatccgggacagggcgagagacccagtacttcgggccaggcacgcggctcctggtgctcg

SEQ ID NO: 963 - protein, 114 aa, Homo sapiens
DSGVTQTPKHLITATGQRVTLRCSPRSGDLSVYWYQQSLDQGLQFLIHYYNGEERAKGNILERFSAQQFPDLHSELNLSSLELGDSALYFCASSLSGTGRETQYFGPGTRLLVL

SEQ ID NO: 964 - protein, 15 aa, Homo sapiens
CASSLSGTGRETQYF

SEQ ID NO: 965 - DNA, 331 bp, Homo sapiens
  source 1..331: mol_type = other DNA
gaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgccgatagatagcaactatcagttaatctggggcgctgggaccaagctaattataaagccag

SEQ ID NO: 966 - protein, 110 aa, Homo sapiens
EDQVTQSPEALRLQEGESSSLNCSYTVSGLRGLFWYRQDPGKGPEFLFTLYSAGEEKEKERLKATLTKKESFLHITAPKPEDSATYLCAVPIDSNYQLIWGAGTKLIIKP

SEQ ID NO: 967 - protein, 13 aa, Homo sapiens
CAVPIDSNYQLIW

SEQ ID NO: 968 - DNA, 346 bp, Homo sapiens
  source 1..346: mol_type = other DNA
gaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttatttctgtgccagcagcccgtactttgggtcctacaatgagcagttcttcgggccagggacacggctcaccgtgctag

SEQ ID NO: 969 - protein, 115 aa, Homo sapiens
EAGVTQFPSHSVIEKGQTVTLRCDPISGHDNLYWYRRVMGKEIKFLLHFVKESKQDESGMPNNRFLAERTGGTYSTLKVQPAELEDSGVYFCASSPYFGSYNEQFFGPGTRLTVL

SEQ ID NO: 970 - protein, 15 aa, Homo sapiens
CASSPYFGSYNEQFF

SEQ ID NO: 971 - DNA, 337 bp, Homo sapiens
  source 1..337: mol_type = other DNA
gagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgtgccgtggacatgcggcagggagcccagaagctggtatttggccaaggaaccaggctgactatcaacccaa

SEQ ID NO: 972 - protein, 112 aa, Homo sapiens
ELKVEQNPLFLSMQEGKNYTIYCNYSTTSDRLYWYRQDPGKSLESLFVLLSNGAVKQEGRLMASLDTKARLSTLHITAAVHDLSATYFCAVDMRQGAQKLVFGQGTRLTINP

SEQ ID NO: 973 - protein, 5 aa, Homo sapiens
TTSDR

SEQ ID NO: 974 - protein, 7 aa, Homo sapiens
LLSNGAV

SEQ ID NO: 975 - protein, 14 aa, Homo sapiens
CAVDMRQGAQKLVF

SEQ ID NO: 976 - protein, 26 aa, Homo sapiens
ELKVEQNPLFLSMQEGKNYTIYCNYS

SEQ ID NO: 977 - protein, 17 aa, Homo sapiens
LYWYRQDPGKSLESLFV

SEQ ID NO: 978 - protein, 33 aa, Homo sapiens
KQEGRLMASLDTKARLSTLHITAAVHDLSATYF

SEQ ID NO: 979 - DNA, 334 bp, Homo sapiens
  source 1..334: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttggggtcctacgagcagtacttcgggccgggcaccaggctcacggtcacag

SEQ ID NO: 980 - protein, 111 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGSYEQYFGPGTRLTVT

SEQ ID NO: 981 - protein, 12 aa, Homo sapiens
CASSLGSYEQYF

SEQ ID NO: 982 - DNA, 331 bp, Homo sapiens
  source 1..331: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgcccctctctatggtcagaattttgtctttggtcccggaaccagattgtccgtgctgccct

SEQ ID NO: 983 - protein, 110 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAPLYGQNFVFGPGTRLSVLP

SEQ ID NO: 984 - protein, 11 aa, Homo sapiens
CAPLYGQNFVF

SEQ ID NO: 985 - DNA, 340 bp, Homo sapiens
  source 1..340: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttgggcagtgggataggtgagcagtacttcgggccgggcaccaggctcacggtcacag

SEQ ID NO: 986 - protein, 113 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLGSGIGEQYFGPGTRLTVT

SEQ ID NO: 987 - protein, 14 aa, Homo sapiens
CASSLGSGIGEQYF

SEQ ID NO: 988 - DNA, 328 bp, Homo sapiens
  source 1..328: mol_type = other DNA
gcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagactcagccatgtattactgtgctctaattatgtacaccggtaaccagttctattttgggacagggacaagtttgacggtcattccaa

SEQ ID NO: 989 - protein, 109 aa, Homo sapiens
AQRVTQPEKLLSVFKGAPVELKCNYSYSGSPELFWYVQYSRQRLQLLLRHISRESIKGFTADLNKGETSFHLKKPFAQEEDSAMYYCALIMYTGNQFYFGTGTSLTVIP

SEQ ID NO: 990 - protein, 13 aa, Homo sapiens
CALIMYTGNQFYF

SEQ ID NO: 991 - DNA, 340 bp, Homo sapiens
  source 1..340: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcactattataatggagaagagagagcaaaaggaaacattcttgaacgattctccgcacaacagttccctgacttgcactctgaactaaacctgagctctctggagctgggggactcagctttgtatttctgtgccagcagcgggggacaaaccgtctatggctacaccttcggttcggggaccaggttaaccgttgtag

SEQ ID NO: 992 - protein, 113 aa, Homo sapiens
DSGVTQTPKHLITATGQRVTLRCSPRSGDLSVYWYQQSLDQGLQFLIHYYNGEERAKGNILERFSAQQFPDLHSELNLSSLELGDSALYFCASSGGQTVYGYTFGSGTRLTVV

SEQ ID NO: 993 - protein, 14 aa, Homo sapiens
CASSGGQTVYGYTF

SEQ ID NO: 994 - DNA, 328 bp, Homo sapiens
  source 1..328: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtgtggccggatagcagctataaattgatcttcgggagtgggaccagactgctggtcaggcctg

SEQ ID NO: 995 - protein, 109 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVWPDSSYKLIFGSGTRLLVRP

SEQ ID NO: 996 - protein, 13 aa, Homo sapiens
CAVWPDSSYKLIF

SEQ ID NO: 997 - DNA, 352 bp, Homo sapiens
  source 1..352: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatcccctggacagggccctatgggcgacgagcagtacttcgggccgggcaccaggctcacggtcacag

SEQ ID NO: 998 - protein, 117 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLSPGQGPMGDEQYFGPGTRLTVT

SEQ ID NO: 999 - protein, 18 aa, Homo sapiens
CASSLSPGQGPMGDEQYF

SEQ ID NO: 1000 - DNA, 337 bp, Homo sapiens
  source 1..337: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctggttttacctcaggaacctacaaatacatctttggaacaggcaccaggctgaaggttttagcaa

SEQ ID NO: 1001 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAGFTSGTYKYIFGTGTRLKVLA

SEQ ID NO: 1002 - protein, 13 aa, Homo sapiens
CAGFTSGTYKYIF

SEQ ID NO: 1003 - DNA, 346 bp, Homo sapiens
  source 1..346: mol_type = other DNA
ggtgctggagtctcccagtctcccaggtacaaagtcacaaagaggggacaggatgtaactctcaggtgtgatccaatttcgagtcatgcaaccctttattggtatcaacaggccctggggcagggcccagagtttctgacttacttcaattatgaagctcaaccagacaaatcagggctgcccagtgatcggttctctgcagagaggcctgagggatccatctccactctgacgattcagcgcacagagcagcgggactcagccatgtatcgctgtgctagcagcccttggactagcgggagtattgagcagttcttcgggccagggacacggctcaccgtgctag

SEQ ID NO: 1004 - protein, 115 aa, Homo sapiens
GAGVSQSPRYKVTKRGQDVTLRCDPISSHATLYWYQQALGQGPEFLTYFNYEAQPDKSGLPSDRFSAERPEGSISTLTIQRTEQRDSAMYRCASSPWTSGSIEQFFGPGTRLTVL

SEQ ID NO: 1005 - protein, 5 aa, Homo sapiens
SSHAT

SEQ ID NO: 1006 - protein, 15 aa, Homo sapiens
CASSPWTSGSIEQFF

SEQ ID NO: 1007 - protein, 26 aa, Homo sapiens
GAGVSQSPRYKVTKRGQDVTLRCDPI

SEQ ID NO: 1008 - protein, 17 aa, Homo sapiens
LYWYQQALGQGPEFLTY

SEQ ID NO: 1009 - protein, 37 aa, Homo sapiens
PDKSGLPSDRFSAERPEGSISTLTIQRTEQRDSAMYR

SEQ ID NO: 1010 - DNA, 325 bp, Homo sapiens
  source 1..325: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgttgactcaggaacctacaaatacatctttggaacaggcaccaggctgaaggttttagcaa

SEQ ID NO: 1011 - protein, 108 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVDSGTYKYIFGTGTRLKVLA

SEQ ID NO: 1012 - protein, 12 aa, Homo sapiens
CAVDSGTYKYIF

SEQ ID NO: 1013 - DNA, 337 bp, Homo sapiens
  source 1..337: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgtgggtgatcacgagcagtacttcgggccgggcaccaggctcacggtcacag

SEQ ID NO: 1014 - protein, 112 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVVGDHEQYFGPGTRLTVT

SEQ ID NO: 1015 - protein, 11 aa, Homo sapiens
CSVVGDHEQYF

SEQ ID NO: 1016 - DNA, 334 bp, Homo sapiens
  source 1..334: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtccccggggggaaaaacaatgacatgcgctttggagcagggaccagactgacagtaaaaccaa

SEQ ID NO: 1017 - protein, 111 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVPGGKNNDMRFGAGTRLTVKP

SEQ ID NO: 1018 - protein, 13 aa, Homo sapiens
CAVPGGKNNDMRF

SEQ ID NO: 1019 - DNA, 349 bp, Homo sapiens
  source 1..349: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcccgaaggacgtcagtactggggccaacgtcctgactttcggggccggcagcaggctgaccgtgctgg

SEQ ID NO: 1020 - protein, 116 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSPKDVSTGANVLTFGAGSRLTVL

SEQ ID NO: 1021 - protein, 17 aa, Homo sapiens
CASSPKDVSTGANVLTF

SEQ ID NO: 1022 - DNA, 328 bp, Homo sapiens
  source 1..328: mol_type = other DNA
gatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgaaaacctcctacgacaaggtgatatttgggccagggacaagcttatcagtcattccaa

SEQ ID NO: 1023 - protein, 109 aa, Homo sapiens
DAKTTQPNSMESNEEEPVHLPCNHSTISGTDYIHWYRQLPSQGPEYVIHGLTSNVNNRMASLAIAEDRKSSTLILHRATLRDAAVYYCILKTSYDKVIFGPGTSLSVIP

SEQ ID NO: 1024 - protein, 12 aa, Homo sapiens
CILKTSYDKVIF

SEQ ID NO: 1025 - protein, 10 aa, Homo sapiens
GPGTSLSVIP

SEQ ID NO: 1026 - DNA, 346 bp, Homo sapiens
  source 1..346: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttagagggtagcggaagtgccggggagctgttttttggagaaggctctaggctgaccgtactgg

SEQ ID NO: 1027 - protein, 115 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLEGSGSAGELFFGEGSRLTVL

SEQ ID NO: 1028 - protein, 16 aa, Homo sapiens
CASSLEGSGSAGELFF

SEQ ID NO: 1029 - DNA, 340 bp, Homo sapiens
  source 1..340: mol_type = other DNA
agccaaaagatagaacagaattccgaggccctgaacattcaggagggtaaaacggccaccctgacctgcaactatacaaactattctccagcatacttacagtggtaccgacaagatccaggaagaggccctgttttcttgctactcatacgtgaaaatgagaaagaaaaaaggaaagaaagactgaaggtcacctttgataccacccttaaacagagtttgtttcatatcacagcctcccagcctgcagactcagctacctacctctgtgctctagaagaaactgacagctgggggaaattgcagtttggagcagggacccaggttgtggtcaccccag

SEQ ID NO: 1030 - protein, 113 aa, Homo sapiens
SQKIEQNSEALNIQEGKTATLTCNYTNYSPAYLQWYRQDPGRGPVFLLLIRENEKEKRKERLKVTFDTTLKQSLFHITASQPADSATYLCALEETDSWGKLQFGAGTQVVVTP

SEQ ID NO: 1031 - protein, 14 aa, Homo sapiens
CALEETDSWGKLQF

SEQ ID NO: 1032 - DNA, 343 bp, Homo sapiens
  source 1..343: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagccggcgaagcttgagctcctacaatgagcagttcttcgggccagggacacggctcaccgtgctag

SEQ ID NO: 1033 - protein, 114 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASRRSLSSYNEQFFGPGTRLTVL

SEQ ID NO: 1034 - protein, 15 aa, Homo sapiens
CASRRSLSSYNEQFF

SEQ ID NO: 1035 - DNA, 340 bp, Homo sapiens
  source 1..340: mol_type = other DNA
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaacctataataatgcaggcaacatgctcacctttggagggggaacaaggttaatggtcaaacccc

SEQ ID NO: 1036 - protein, 113 aa, Homo sapiens
DQQVKQNSPSLSVQEGRISILNCDYTNSMFDYFLWYKKYPAEGPTFLISISSIKDKNEDGRFTVFLNKSAKHLSLHIVPSQPGDSAVYFCAATYNNAGNMLTFGGGTRLMVKP

SEQ ID NO: 1037 - protein, 14 aa, Homo sapiens
CAATYNNAGNMLTF

SEQ ID NO: 1038 - DNA, 355 bp, Homo sapiens
  source 1..355: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtagggagtgtggggggactagcggacttcaatgagcagttcttcgggccagggacacggctcaccgtgctag

SEQ ID NO: 1039 - protein, 118 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSVGSVGGLADFNEQFFGPGTRLTVL

SEQ ID NO: 1040 - protein, 19 aa, Homo sapiens
CASSVGSVGGLADFNEQFF

SEQ ID NO: 1041 - DNA, 343 bp, Homo sapiens
  source 1..343: mol_type = other DNA
gcccagaagataactcaaacccaaccaggaatgttcgtgcaggaaaaggaggctgtgactctggactgcacatatgacaccagtgatcaaagttatggtctattctggtacaagcagcccagcagtggggaaatgatttttcttatttatcaggggtcttatgacgagcaaaatgcaacagaaggtcgctactcattgaatttccagaaggcaagaaaatccgccaaccttgtcatctccgcttcacaactgggggactcagcaatgtatttctgtgcaatgagagagggctgggttagtgacatgcgctttggagcagggaccagactgacagtaaaaccaa

SEQ ID NO: 1042 - protein, 114 aa, Homo sapiens
AQKITQTQPGMFVQEKEAVTLDCTYDTSDQSYGLFWYKQPSSGEMIFLIYQGSYDEQNATEGRYSLNFQKARKSANLVISASQLGDSAMYFCAMREGWVSDMRFGAGTRLTVKP

SEQ ID NO: 1043 - protein, 13 aa, Homo sapiens
CAMREGWVSDMRF

SEQ ID NO: 1044 - DNA, 334 bp, Homo sapiens
  source 1..334: mol_type = other DNA
aatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttctgtgccagcagttcccctttctacgagcagtacttcgggccgggcaccaggctcacggtcacag

SEQ ID NO: 1045 - protein, 111 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSSPFYEQYFGPGTRLTVT

SEQ ID NO: 1046 - protein, 6 aa, Homo sapiens
SVGEGT

SEQ ID NO: 1047 - protein, 12 aa, Homo sapiens
CASSSPFYEQYF

SEQ ID NO: 1048 - protein, 26 aa, Homo sapiens
NAGVTQTPKFRVLKTGQSMTLLCAQD

SEQ ID NO: 1049 - protein, 17 aa, Homo sapiens
MYWYRQDPGMGLRLIHY

SEQ ID NO: 1050 - protein, 36 aa, Homo sapiens
TAKGEVPDGYNVSRLKKQNFLLGLESAAPSQTSVYF

SEQ ID NO: 1051 - DNA, 331 bp, Homo sapiens
  source 1..331: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggaagaatggggaagccaaggaaatctcatctttggaaaaggcactaaactctctgttaaaccaa

SEQ ID NO: 1052 - protein, 110 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVEEWGSQGNLIFGKGTKLSVKP

SEQ ID NO: 1053 - protein, 14 aa, Homo sapiens
CAVEEWGSQGNLIF

SEQ ID NO: 1054 - DNA, 355 bp, Homo sapiens
  source 1..355: mol_type = other DNA
gaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaaggaagggcgtttctgggccaggggttgtacgagcagtacttcgggccgggcaccaggctcacggtcacag

SEQ ID NO: 1055 - protein, 118 aa, Homo sapiens
ETGVTQTPRHLVMGMTNKKSLKCEQHLGHNAMYWYKQSAKKPLELMFVYSLEERVENNSVPSRFSPECPNSSHLFLHLHTLQPEDSALYLCASSQGRAFLGQGLYEQYFGPGTRLTVT

SEQ ID NO: 1056 - protein, 19 aa, Homo sapiens
CASSQGRAFLGQGLYEQYF

SEQ ID NO: 1057 - DNA, 331 bp, Homo sapiens
  source 1..331: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcaggagtaggaagagatgacaagatcatctttggaaaagggacacgacttcatattctcccca

SEQ ID NO: 1058 - protein, 110 aa, Homo sapiens
TQLLEQSPQFLSIQEGENLTVYCNSSSVFSSLQWYRQEPGEGPVLLVTVVTGGEVKKLKRLTFQFGDARKDSSLHITAAQPGDTGLYLCAGVGRDDKIIFGKGTRLHILP

SEQ ID NO: 1059 - protein, 12 aa, Homo sapiens
CAGVGRDDKIIF

SEQ ID NO: 1060 - DNA, 346 bp, Homo sapiens
  source 1..346: mol_type = other DNA
ggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagatcggggggcctacgagcagtacttcgggccgggcaccaggctcacggtcacag

SEQ ID NO: 1061 - protein, 115 aa, Homo sapiens
GAVVSQHPSRVICKSGTSVKIECRSLDFQATTMFWYRQFPKQSLMLMATSNEGSKATYEQGVEKDKFLINHASLTLSTLTVTSAHPEDSSFYICSARDRGAYEQYFGPGTRLTVT

SEQ ID NO: 1062 - protein, 13 aa, Homo sapiens
CSARDRGAYEQYF

SEQ ID NO: 1063 - protein, 26 aa, Homo sapiens
GAVVSQHPSRVICKSGTSVKIECRSL

SEQ ID NO: 1064 - DNA, 340 bp, Homo sapiens
  source 1..340: mol_type = other DNA
ggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgagtgatctgaataacaatgccagactcatgtttggagatggaactcagctggtggtgaagccca

SEQ ID NO: 1065 - protein, 113 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSDLNNNARLMFGDGTQLVVKP

SEQ ID NO: 1066 - protein, 14 aa, Homo sapiens
CALSDLNNNARLMF

SEQ ID NO: 1067 - DNA, 349 bp, Homo sapiens
  source 1..349: mol_type = other DNA
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgcgagcagcggggggttctacaatgagcagttcttcgggccagggacacggctcaccgtgctag

SEQ ID NO: 1068 - protein, 116 aa, Homo sapiens
EPEVTQTPSHQVTQMGQEVILRCVPISNHLYFYWYRQILGQKVEFLVSFYNNEISEKSEIFDDQFSVERPDGSNFTLKIRSTKLEDSAMYFCASSASSGGFYNEQFFGPGTRLTVL

SEQ ID NO: 1069 - protein, 16 aa, Homo sapiens
CASSASSGGFYNEQFF

SEQ ID NO: 1070 - DNA, 346 bp, Homo sapiens
  source 1..346: mol_type = other DNA
gcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgtgccgtgaatggggcaggaggaggtgctgacggactcacctttggcaaagggactcatctaatcatccagccct

SEQ ID NO: 1071 - protein, 115 aa, Homo sapiens
AQSVSQHNHHVILSEAASLELGCNYSYGGTVNLFWYVQYPGQHLQLLLKYFSGDPLVKGIKGFEAEFIKSKFSFNLRKPSVQWSDTAEYFCAVNGAGGGADGLTFGKGTHLIIQP

SEQ ID NO: 1072 - protein, 6 aa, Homo sapiens
YGGTVN

SEQ ID NO: 1073 - protein, 8 aa, Homo sapiens
YFSGDPLV

SEQ ID NO: 1074 - protein, 15 aa, Homo sapiens
CAVNGAGGGADGLTF

SEQ ID NO: 1075 - protein, 26 aa, Homo sapiens
AQSVSQHNHHVILSEAASLELGCNYS

SEQ ID NO: 1076 - protein, 17 aa, Homo sapiens
LFWYVQYPGQHLQLLLK

SEQ ID NO: 1077 - protein, 33 aa, Homo sapiens
KGIKGFEAEFIKSKFSFNLRKPSVQWSDTAEYF

SEQ ID NO: 1078 - DNA, 346 bp, Homo sapiens
  source 1..346: mol_type = other DNA
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagtgtccgcggtagggaacattcagtacttcggcgccgggacccggctctcagtgctgg

SEQ ID NO: 1079 - protein, 115 aa, Homo sapiens
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVSAVGNIQYFGAGTRLSVL

SEQ ID NO: 1080 - protein, 15 aa, Homo sapiens
CASSLVSAVGNIQYF

SEQ ID NO: 1081 - protein, 9 aa, Homo sapiens
GAGTRLSVL

SEQ ID NO: 1082 - DNA, 334 bp, Homo sapiens
  source 1..334: mol_type = other DNA
cagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgagcaattataacaccgacaagctcatctttgggactgggaccagattacaagtctttccaa

SEQ ID NO: 1083 - protein, 111 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMSNYNTDKLIFGTGTRLQVFP

SEQ ID NO: 1084 - protein, 13 aa, Homo sapiens
CAMSNYNTDKLIF

SEQ ID NO: 1085 - DNA, 349 bp, Homo sapiens
  source 1..349: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtaccaccgggggggacagtcttagcaatcagccccagcattttggtgatgggactcgactctccatcctag

SEQ ID NO: 1086 - protein, 116 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASTTGGDSLSNQPQHFGDGTRLSIL

SEQ ID NO: 1087 - protein, 17 aa, Homo sapiens
CASTTGGDSLSNQPQHF

SEQ ID NO: 1088 - DNA, 352 bp, Homo sapiens
  source 1..352: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaaacgccccagggatgctggtggtactagctatggaaagctgacatttggacaagggaccatcttgactgtccatccaa

SEQ ID NO: 1089 - protein, 117 aa, Homo sapiens
QKEVEQNSGPLSVPEGAIASLNCTYSDRGSQSFFWYRQYSGKSPELIMFIYSNGDKEDGRFTAQLNKASQYVSLLIRDSQPSDSATYLCAVKRPRDAGGTSYGKLTFGQGTILTVHP

SEQ ID NO: 1090 - protein, 19 aa, Homo sapiens
CAVKRPRDAGGTSYGKLTF

SEQ ID NO: 1091 - DNA, 340 bp, Homo sapiens
  source 1..340: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactggacagggacctacgagcagtacttcgggccgggcaccaggctcacggtcacag

SEQ ID NO: 1092 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYWTGTYEQYFGPGTRLTVT

SEQ ID NO: 1093 - protein, 14 aa, Homo sapiens
CASSYWTGTYEQYF

SEQ ID NO: 1094 - DNA, 340 bp, Homo sapiens
  source 1..340: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggtctattcaggaggaggtgctgacggactcacctttggcaaagggactcatctaatcatccagccct

SEQ ID NO: 1095 - protein, 113 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATVYSGGGADGLTFGKGTHLIIQP

SEQ ID NO: 1096 - protein, 15 aa, Homo sapiens
CATVYSGGGADGLTF

SEQ ID NO: 1097 - DNA, 337 bp, Homo sapiens
  source 1..337: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtagcggaacctacaatgagcagttcttcgggccagggacacggctcaccgtgctag

SEQ ID NO: 1098 - protein, 112 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSGTYNEQFFGPGTRLTVL

SEQ ID NO: 1099 - protein, 13 aa, Homo sapiens
CASSSGTYNEQFF

SEQ ID NO: 1100 - DNA, 331 bp, Homo sapiens
  source 1..331: mol_type = other DNA
gcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagactcagccatgtattactgtgcttcgccgagcaaagctgcaggcaacaagctaacttttggaggaggaaccagggtgctagttaaaccaa

SEQ ID NO: 1101 - protein, 110 aa, Homo sapiens
AQRVTQPEKLLSVFKGAPVELKCNYSYSGSPELFWYVQYSRQRLQLLLRHISRESIKGFTADLNKGETSFHLKKPFAQEEDSAMYYCASPSKAAGNKLTFGGGTRVLVKP

SEQ ID NO: 1102 - protein, 14 aa, Homo sapiens
CASPSKAAGNKLTF

SEQ ID NO: 1103 - protein, 10 aa, Homo sapiens
GGGTRVLVKP

SEQ ID NO: 1104 - DNA, 352 bp, Homo sapiens
  source 1..352: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagaggccagggccccctctggaaacaccatatattttggagagggaagttggctcactgttgtag

SEQ ID NO: 1105 - protein, 117 aa, Homo sapiens
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSLEARAPSGNTIYFGEGSWLTVV

SEQ ID NO: 1106 - protein, 17 aa, Homo sapiens
CASSLEARAPSGNTIYF

SEQ ID NO: 1107 - DNA, 349 bp, Homo sapiens
  source 1..349: mol_type = other DNA
gctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctatgggagggtcagggcggatctgaaaagctggtctttggaaagggaacgaaactgacagtaaacccat

SEQ ID NO: 1108 - protein, 116 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALWEGQGGSEKLVFGKGTKLTVNP

SEQ ID NO: 1109 - protein, 15 aa, Homo sapiens
CALWEGQGGSEKLVF

SEQ ID NO: 1110 - DNA, 340 bp, Homo sapiens
  source 1..340: mol_type = other DNA
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccaccacactcccgggggacactgaagctttctttggacaaggcaccagactcacagttgtag

SEQ ID NO: 1111 - protein, 113 aa, Homo sapiens
DTGVSQNPRHKITKRGQNVTFRCDPISEHNRLYWYRQTLGQGPEFLTYFQNEAQLEKSRLLSDRFSAERPKGSFSTLEIQRTEQGDSAMYLCATTLPGDTEAFFGQGTRLTVV

SEQ ID NO: 1112 - protein, 13 aa, Homo sapiens
CATTLPGDTEAFF

SEQ ID NO: 1113 - DNA, 343 bp, Homo sapiens
  source 1..343: mol_type = other DNA
gcccagacagtcactcagtctcaaccagagatgtctgtgcaggaggcagagactgtgaccctgagttgcacatatgacaccagtgagagtaattattatttgttctggtacaaacagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcatgaaggggagagatgacaagatcatctttggaaaagggacacgacttcatattctcccca

SEQ ID NO: 1114 - protein, 114 aa, Homo sapiens
AQTVTQSQPEMSVQEAETVTLSCTYDTSESNYYLFWYKQPPSRQMILVIRQEAYKQQNATENRFSVNFQKAAKSFSLKISDSQLGDTAMYFCAFMKGRDDKIIFGKGTRLHILP

SEQ ID NO: 1115 - protein, 7 aa, Homo sapiens
TSESNYY

SEQ ID NO: 1116 - protein, 13 aa, Homo sapiens
CAFMKGRDDKIIF

SEQ ID NO: 1117 - DNA, 340 bp, Homo sapiens
  source 1..340: mol_type = other DNA
gatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttggaccgccatcagccccagcattttggtgatgggactcgactctccatcctag

SEQ ID NO: 1118 - protein, 113 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSLDRHQPQHFGDGTRLSIL

SEQ ID NO: 1119 - protein, 13 aa, Homo sapiens
CASSLDRHQPQHF

SEQ ID NO: 1120 - DNA, 334 bp, Homo sapiens
  source 1..334: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggcaaataatgcaggcaacatgctcacctttggagggggaacaaggttaatggtcaaacccc

SEQ ID NO: 1121 - protein, 111 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATANNAGNMLTFGGGTRLMVKP

SEQ ID NO: 1122 - protein, 13 aa, Homo sapiens
CATANNAGNMLTF

SEQ ID NO: 1123 - DNA, 346 bp, Homo sapiens
  source 1..346: mol_type = other DNA
gacgctggagtcacccaaagtcccacacacctgatcaaaacgagaggacagcacgtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctctgtgccagcagcttggaactagcgggagggcgtgatacgcagtattttggcccaggcacccggctgacagtgctcg

SEQ ID NO: 1124 - protein, 115 aa, Homo sapiens
DAGVTQSPTHLIKTRGQHVTLRCSPISGHKSVSWYQQVLGQGPQFIFQYYEKEERGRGNFPDRFSARQFPNYSSELNVNALLLGDSALYLCASSLELAGGRDTQYFGPGTRLTVL

SEQ ID NO: 1125 - protein, 5 aa, Homo sapiens
SGHKS

SEQ ID NO: 1126 - protein, 6 aa, Homo sapiens
YYEKEE

SEQ ID NO: 1127 - protein, 16 aa, Homo sapiens
CASSLELAGGRDTQYF

SEQ ID NO: 1128 - protein, 26 aa, Homo sapiens
DAGVTQSPTHLIKTRGQHVTLRCSPI

SEQ ID NO: 1129 - protein, 17 aa, Homo sapiens
VSWYQQVLGQGPQFIFQ

SEQ ID NO: 1130 - protein, 36 aa, Homo sapiens
RGRGNFPDRFSARQFPNYSSELNVNALLLGDSALYL

SEQ ID NO: 1131 - DNA, 346 bp, Homo sapiens
  source 1..346: mol_type = other DNA
gcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaacctgaggaaaccctctgtgcattggagtgatgctgctgagtacttctgtgctgtgggtgccggaaccggcactgccagtaaactcacctttgggactggaacaagacttcaggtcacgctcg

SEQ ID NO: 1132 - protein, 115 aa, Homo sapiens
AQSVTQPDIHITVSEGASLELRCNYSYGATPYLFWYVQSPGQGLQLLLKYFSGDTLVQGIKGFEAEFKRSQSSFNLRKPSVHWSDAAEYFCAVGAGTGTASKLTFGTGTRLQVTL

SEQ ID NO: 1133 - protein, 15 aa, Homo sapiens
CAVGAGTGTASKLTF

SEQ ID NO: 1134 - protein, 10 aa, Homo sapiens
GTGTRLQVTL

SEQ ID NO: 1135 - DNA, 340 bp, Homo sapiens
  source 1..340: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgaggggacagtccttgacgagcagtacttcgggccgggcaccaggctcacggtcacag

SEQ ID NO: 1136 - protein, 113 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSEGTVLDEQYFGPGTRLTVT

SEQ ID NO: 1137 - protein, 14 aa, Homo sapiens
CASSEGTVLDEQYF

SEQ ID NO: 1138 - DNA, 352 bp, Homo sapiens
  source 1..352: mol_type = other DNA
gctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaaggatctaactttggaaatgagaaattaacctttgggactggaacaagactcaccatcataccca

SEQ ID NO: 1139 - protein, 117 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEGSNFGNEKLTFGTGTRLTIIP

SEQ ID NO: 1140 - protein, 16 aa, Homo sapiens
CALSEGSNFGNEKLTF

SEQ ID NO: 1141 - DNA, 334 bp, Homo sapiens
  source 1..334: mol_type = other DNA
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttggggtcgtctacgcagtattttggcccaggcacccggctgacagtgctcg

SEQ ID NO: 1142 - protein, 111 aa, Homo sapiens
EAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGSSTQYFGPGTRLTVL

SEQ ID NO: 1143 - protein, 12 aa, Homo sapiens
CASSLGSSTQYF

SEQ ID NO: 1144 - DNA, 346 bp, Homo sapiens
  source 1..346: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgcgggggggaattatggaggaagccaaggaaatctcatctttggaaaaggcactaaactctctgttaaaccaa

SEQ ID NO: 1145 - protein, 115 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAAGGNYGGSQGNLIFGKGTKLSVKP

SEQ ID NO: 1146 - protein, 16 aa, Homo sapiens
CAAGGNYGGSQGNLIF

SEQ ID NO: 1147 - DNA, 355 bp, Homo sapiens
  source 1..355: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttggagcctagcggggggcggaggtgggacaatgagcagttcttcgggccagggacacggctcaccgtgctag

SEQ ID NO: 1148 - protein, 118 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSLEPSGGRRWDNEQFFGPGTRLTVL

SEQ ID NO: 1149 - protein, 19 aa, Homo sapiens
CASSLEPSGGRRWDNEQFF

SEQ ID NO: 1150 - DNA, 358 bp, Homo sapiens
  source 1..358: mol_type = other DNA
ggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgactgggccccaagctaatgctggtggtactagctatggaaagctgacatttggacaagggaccatcttgactgtccatccaa

SEQ ID NO: 1151 - protein, 119 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALTGPQANAGGTSYGKLTFGQGTILTVHP

SEQ ID NO: 1152 - protein, 20 aa, Homo sapiens
CALTGPQANAGGTSYGKLTF

SEQ ID NO: 1153 - DNA, 334 bp, Homo sapiens
  source 1..334: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcgaggatacctatggctacaccttcggttcggggaccaggttaaccgttgtag

SEQ ID NO: 1154 - protein, 111 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSEDTYGYTFGSGTRLTVV

SEQ ID NO: 1155 - protein, 12 aa, Homo sapiens
CASSEDTYGYTF

SEQ ID NO: 1156 - DNA, 334 bp, Homo sapiens
  source 1..334: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgatcgtcaccggtaaccagttctattttgggacagggacaagtttgacggtcattccaa

SEQ ID NO: 1157 - protein, 111 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVIVTGNQFYFGTGTSLTVIP

SEQ ID NO: 1158 - protein, 12 aa, Homo sapiens
CAVIVTGNQFYF

SEQ ID NO: 1159 - DNA, 343 bp, Homo sapiens
  source 1..343: mol_type = other DNA
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcttcctaactgactcctacaatgagcagttcttcgggccagggacacggctcaccgtgctag

SEQ ID NO: 1160 - protein, 114 aa, Homo sapiens
DAGVTQSPTHLIKTRGQQVTLRCSPKSGHDTVSWYQQALGQGPQFIFQYYEEEERQRGNFPDRFSGHQFPNYSSELNVNALLLGDSALYLCASSFLTDSYNEQFFGPGTRLTVL

SEQ ID NO: 1161 - protein, 15 aa, Homo sapiens
CASSFLTDSYNEQFF

SEQ ID NO: 1162 - DNA, 340 bp, Homo sapiens
  source 1..340: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggactaccggacaggaggaagctacatacctacatttggaagaggaaccagccttattgttcatccgt

SEQ ID NO: 1163 - protein, 113 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDYRTGGSYIPTFGRGTSLIVHP

SEQ ID NO: 1164 - protein, 15 aa, Homo sapiens
CATDYRTGGSYIPTF

SEQ ID NO: 1165 - DNA, 349 bp, Homo sapiens
  source 1..349: mol_type = other DNA
gaagctgaagttgcccagtcccccagatataagattacagagaaaagccaggctgtggctttttggtgtgatcctatttctggccatgctaccctttactggtaccggcagatcctgggacagggcccggagcttctggttcaatttcaggatgagagtgtagtagatgattcacagttgcctaaggatcgattttctgcagagaggctcaaaggagtagactccactctcaagatccagcctgcagagcttggggactcggccatgtatctctgtgccagcagctacctagcgggagggtttagggatgagcagttcttcgggccagggacacggctcaccgtgctag

SEQ ID NO: 1166 - protein, 116 aa, Homo sapiens
EAEVAQSPRYKITEKSQAVAFWCDPISGHATLYWYRQILGQGPELLVQFQDESVVDDSQLPKDRFSAERLKGVDSTLKIQPAELGDSAMYLCASSYLAGGFRDEQFFGPGTRLTVL

SEQ ID NO: 1167 - protein, 6 aa, Homo sapiens
FQDESV

SEQ ID NO: 1168 - protein, 16 aa, Homo sapiens
CASSYLAGGFRDEQFF

SEQ ID NO: 1169 - protein, 26 aa, Homo sapiens
EAEVAQSPRYKITEKSQAVAFWCDPI

SEQ ID NO: 1170 - protein, 17 aa, Homo sapiens
LYWYRQILGQGPELLVQ

SEQ ID NO: 1171 - protein, 37 aa, Homo sapiens
VDDSQLPKDRFSAERLKGVDSTLKIQPAELGDSAMYL

SEQ ID NO: 1172 - DNA, 340 bp, Homo sapiens
  source 1..340: mol_type = other DNA
ggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgagtggcagggaatatggaaacaagctggtctttggcgcaggaaccattctgagagtcaagtcct

SEQ ID NO: 1173 - protein, 113 aa, Homo sapiens
GNSVTQMEGPVTLSEEAFLTINCTYTATGYPSLFWYVQYPGEGLQLLLKATKADDKGSNKGFEATYRKETTSFHLEKGSVQVSDSAVYFCALSGREYGNKLVFGAGTILRVKS

SEQ ID NO: 1174 - protein, 14 aa, Homo sapiens
CALSGREYGNKLVF

SEQ ID NO: 1175 - DNA, 337 bp, Homo sapiens
  source 1..337: mol_type = other DNA
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagccccccggacagctatggctacaccttcggttcggggaccaggttaaccgttgtag

SEQ ID NO: 1176 - protein, 112 aa, Homo sapiens
DSGVTQTPKHLITATGQRVTLRCSPRSGDLSVYWYQQSLDQGLQFLIQYYNGEERAKGNILERFSAQQFPDLHSELNLSSLELGDSALYFCASSPPDSYGYTFGSGTRLTVV

SEQ ID NO: 1177 - protein, 13 aa, Homo sapiens
CASSPPDSYGYTF

SEQ ID NO: 1178 - DNA, 325 bp, Homo sapiens
  source 1..325: mol_type = other DNA
ggacaaaacattgaccagcccactgagatgacagctacggaaggtgccattgtccagatcaactgcacgtaccagacatctgggttcaacgggctgttctggtaccagcaacatgctggcgaagcacctacatttctgtcttacaatgttctggatggtttggaggagaaaggtcgtttttcttcattccttagtcggtctaaagggtacagttacctccttttgaaggagctccagatgaaagactctgcctcttacctctgtgctgtgaggggcgcaacaaagctcagctttggagccggaaccacagtaactgtaagagcaa

SEQ ID NO: 1179 - protein, 108 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVRGATKLSFGAGTTVTVRA

SEQ ID NO: 1180 - protein, 11 aa, Homo sapiens
CAVRGATKLSF

SEQ ID NO: 1181 - protein, 10 aa, Homo sapiens
GAGTTVTVRA

SEQ ID NO: 1182 - DNA, 337 bp, Homo sapiens
  source 1..337: mol_type = other DNA
aatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttctgtgccagcagtcccgtcggttggcgcgagcagttcttcgggccagggacacggctcaccgtgctag

SEQ ID NO: 1183 - protein, 112 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSPVGWREQFFGPGTRLTVL

SEQ ID NO: 1184 - protein, 13 aa, Homo sapiens
CASSPVGWREQFF

SEQ ID NO: 1185 - DNA, 337 bp, Homo sapiens
  source 1..337: mol_type = other DNA
ggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagagcccgtcaggaggaagctacatacctacatttggaagaggaaccagccttattgttcatccgt

SEQ ID NO: 1186 - protein, 112 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAESPSGGSYIPTFGRGTSLIVHP

SEQ ID NO: 1187 - protein, 14 aa, Homo sapiens
CAESPSGGSYIPTF

SEQ ID NO: 1188 - DNA, 343 bp, Homo sapiens
  source 1..343: mol_type = other DNA
ggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaggcccccaggggggggctacaccttcggttcggggaccaggttaaccgttgtag

SEQ ID NO: 1189 - protein, 114 aa, Homo sapiens
GAVVSQHPSRVICKSGTSVKIECRSLDFQATTMFWYRQFPKQSLMLMATSNEGSKATYEQGVEKDKFLINHASLTLSTLTVTSAHPEDSSFYICSARPPGGGYTFGSGTRLTVV

SEQ ID NO: 1190 - protein, 12 aa, Homo sapiens
CSARPPGGGYTF

SEQ ID NO: 1191 - DNA, 334 bp, Homo sapiens
  source 1..334: mol_type = other DNA
atactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccttcgattcaggatacagcaccctcacctttgggaaggggactatgcttctagtctctccag

SEQ ID NO: 1192 - protein, 111 aa, Homo sapiens
ILNVEQSPQSLHVQEGDSTNFTCSFPSSNFYALHWYRWETAKSPEALFVMTLNGDEKKKGRISATLNTKEGYSYLYIKGSQPEDSATYLCAFDSGYSTLTFGKGTMLLVSP

SEQ ID NO: 1193 - protein, 12 aa, Homo sapiens
CAFDSGYSTLTF

SEQ ID NO: 1194 - DNA, 352 bp, Homo sapiens
  source 1..352: mol_type = other DNA
gatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttccagactgtcgggtcagggtcctacgagcagtacttcgggccgggcaccaggctcacggtcacag

SEQ ID NO: 1195 - protein, 117 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSFQTVGSGSYEQYFGPGTRLTVT

SEQ ID NO: 1196 - protein, 17 aa, Homo sapiens
CASSFQTVGSGSYEQYF

SEQ ID NO: 1197 - DNA, 355 bp, Homo sapiens
  source 1..355: mol_type = other DNA
gctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggtctcctctggggctgggagttaccaactcactttcgggaaggggaccaaactctcggtcataccaa

SEQ ID NO: 1198 - protein, 118 aa, Homo sapiens
AQKVTQAQTEISVVEKEDVTLDCVYETRDTTYYLFWYKQPPSGELVFLIRRNSFDEQNEISGRYSWNFQKSTSSFNFTITASQVVDSAVYFCALSEVSSGAGSYQLTFGKGTKLSVIP

SEQ ID NO: 1199 - protein, 17 aa, Homo sapiens
CALSEVSSGAGSYQLTF

SEQ ID NO: 1200 - DNA, 340 bp, Homo sapiens
  source 1..340: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgacggggagaacactgaagctttctttggacaaggcaccagactcacagttgtag

SEQ ID NO: 1201 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYDGENTEAFFGQGTRLTVV

SEQ ID NO: 1202 - protein, 14 aa, Homo sapiens
CASSYDGENTEAFF

SEQ ID NO: 1203 - DNA, 334 bp, Homo sapiens
  source 1..334: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtacaccgggtgagaaattaacctttgggactggaacaagactcaccatcataccca

SEQ ID NO: 1204 - protein, 111 aa, Homo sapiens
GENVEQHPSTLSVQEGDSAVIKCTYSDSASNYFPWYKQELGKGPQLIIDIRSNVGEKKDQRIAVTLNKTAKHFSLHITETQPEDSAVYFCAASTPGEKLTFGTGTRLTIIP

SEQ ID NO: 1205 - protein, 12 aa, Homo sapiens
CAASTPGEKLTF

SEQ ID NO: 1206 - DNA, 343 bp, Homo sapiens
  source 1..343: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaaaccgactagcgggagcgtgaatgagcagttcttcgggccagggacacggctcaccgtgctag

SEQ ID NO: 1207 - protein, 114 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASKPTSGSVNEQFFGPGTRLTVL

SEQ ID NO: 1208 - protein, 15 aa, Homo sapiens
CASKPTSGSVNEQFF

SEQ ID NO: 1209 - DNA, 337 bp, Homo sapiens
  source 1..337: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgggtaatactggaggcttcaaaactatctttggagcaggaacaagactatttgttaaagcaa

SEQ ID NO: 1210 - protein, 112 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAVGNTGGFKTIFGAGTRLFVKA

SEQ ID NO: 1211 - protein, 13 aa, Homo sapiens
CAVGNTGGFKTIF

SEQ ID NO: 1212 - DNA, 337 bp, Homo sapiens
  source 1..337: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgagggatcactggcccagtacttcgggccaggcacgcggctcctggtgctcg

SEQ ID NO: 1213 - protein, 112 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVEGSLAQYFGPGTRLLVL

SEQ ID NO: 1214 - protein, 11 aa, Homo sapiens
CSVEGSLAQYF

SEQ ID NO: 1215 - DNA, 331 bp, Homo sapiens
  source 1..331: mol_type = other DNA
gagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgtgccgtggacagggaccaaggaaatctcatctttggaaaaggcactaaactctctgttaaaccaa

SEQ ID NO: 1216 - protein, 110 aa, Homo sapiens
ELKVEQNPLFLSMQEGKNYTIYCNYSTTSDRLYWYRQDPGKSLESLFVLLSNGAVKQEGRLMASLDTKARLSTLHITAAVHDLSATYFCAVDRDQGNLIFGKGTKLSVKP

SEQ ID NO: 1217 - protein, 12 aa, Homo sapiens
CAVDRDQGNLIF

SEQ ID NO: 1218 - DNA, 331 bp, Homo sapiens
  source 1..331: mol_type = other DNA
aatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgacgggggtgagcagttcttcgggccagggacacggctcaccgtgctag

SEQ ID NO: 1219 - protein, 110 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSDGGEQFFGPGTRLTVL

SEQ ID NO: 1220 - protein, 11 aa, Homo sapiens
CASSDGGEQFF

SEQ ID NO: 1221 - DNA, 334 bp, Homo sapiens
  source 1..334: mol_type = other DNA
ggacaaaacattgaccagcccactgagatgacagctacggaaggtgccattgtccagatcaactgcacgtaccagacatctgggttcaacgggctgttctggtaccagcaacatgctggcgaagcacctacatttctgtcttacaatgttctggatggtttggaggagaaaggtcgtttttcttcattccttagtcggtctaaagggtacagttacctccttttgaaggagctccagatgaaagactctgcctcttacctctgtgctgtgagagatggggctagcaactatcagttaatctggggcgctgggaccaagctaattataaagccag

SEQ ID NO: 1222 - protein, 111 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVRDGASNYQLIWGAGTKLIIKP

SEQ ID NO: 1223 - protein, 14 aa, Homo sapiens
CAVRDGASNYQLIW

SEQ ID NO: 1224 - DNA, 337 bp, Homo sapiens
  source 1..337: mol_type = other DNA
gatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagcagtaacgggtacaatgagcagttcttcgggccagggacacggctcaccgtgctag

SEQ ID NO: 1225 - protein, 112 aa, Homo sapiens
DAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSSNGYNEQFFGPGTRLTVL

SEQ ID NO: 1226 - protein, 13 aa, Homo sapiens
CATSSNGYNEQFF

SEQ ID NO: 1227 - DNA, 319 bp, Homo sapiens
  source 1..319: mol_type = other DNA
acccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggagagttttactattttgggacagggacaagtttgacggtcattccaa

SEQ ID NO: 1228 - protein, 106 aa, Homo sapiens
TQLLEQSPQFLSIQEGENLTVYCNSSSVFSSLQWYRQEPGEGPVLLVTVVTGGEVKKLKRLTFQFGDARKDSSLHITAAQPGDTGLYLCAGEFYYFGTGTSLTVIP

SEQ ID NO: 1229 - protein, 8 aa, Homo sapiens
CAGEFYYF

SEQ ID NO: 1230 - DNA, 337 bp, Homo sapiens
  source 1..337: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagccgggagggcgatcagccccagcattttggtgatgggactcgactctccatcctag

SEQ ID NO: 1231 - protein, 112 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSREGDQPQHFGDGTRLSIL

SEQ ID NO: 1232 - protein, 13 aa, Homo sapiens
CASSREGDQPQHF

SEQ ID NO: 1233 - DNA, 322 bp, Homo sapiens
  source 1..322: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtccgctatggtcagaattttgtctttggtcccggaaccagattgtccgtgctgccct

SEQ ID NO: 1234 - protein, 107 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVRYGQNFVFGPGTRLSVLP

SEQ ID NO: 1235 - protein, 11 aa, Homo sapiens
CAVRYGQNFVF

SEQ ID NO: 1236 - DNA, 349 bp, Homo sapiens
  source 1..349: mol_type = other DNA
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgtagtcgggacagcctcctctggaaacaccatatattttggagagggaagttggctcactgttgtag

SEQ ID NO: 1237 - protein, 116 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSVVGTASSGNTIYFGEGSWLTVV

SEQ ID NO: 1238 - protein, 15 aa, Homo sapiens
CSVVGTASSGNTIYF

SEQ ID NO: 1239 - DNA, 337 bp, Homo sapiens
  source 1..337: mol_type = other DNA
gcccagtctgtgagccagcataaccaccatgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgtgccgtgaggggaaccggtaaccagttctattttgggacagggacaagtttgacggtcattccaa

SEQ ID NO: 1240 - protein, 112 aa, Homo sapiens
AQSVSQHNHHVILSEAASLELGCNYSYGGTVNLFWYVQYPGQHLQLLLKYFSGDPLVKGIKGFEAEFIKSKFSFNLRKPSVQWSDTAEYFCAVRGTGNQFYFGTGTSLTVIP

SEQ ID NO: 1241 - protein, 12 aa, Homo sapiens
CAVRGTGNQFYF

SEQ ID NO: 1242 - DNA, 340 bp, Homo sapiens
  source 1..340: mol_type = other DNA
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagccccggacagggaagctatggctacaccttcggttcggggaccaggttaaccgttgtag

SEQ ID NO: 1243 - protein, 113 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPGQGSYGYTFGSGTRLTVV

SEQ ID NO: 1244 - protein, 14 aa, Homo sapiens
CASSPGQGSYGYTF

SEQ ID NO: 1245 - DNA, 340 bp, Homo sapiens
  source 1..340: mol_type = other DNA
gcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttgaaagcatcaacggttttgaggctgaatttaacaagagtcaaacttccttccacttgaggaaaccctcagtccatataagcgacacggctgagtacttctgtgctgtgattccggatagcaactatcagttaatctggggcgctgggaccaagctaattataaagccag

SEQ ID NO: 1246 - protein, 113 aa, Homo sapiens
AQSVTQLDSQVPVFEEAPVELRCNYSSSVSVYLFWYVQYPNQGLQLLLKYLSGSTLVESINGFEAEFNKSQTSFHLRKPSVHISDTAEYFCAVIPDSNYQLIWGAGTKLIIKP

SEQ ID NO: 1247 - protein, 13 aa, Homo sapiens
CAVIPDSNYQLIW

SEQ ID NO: 1248 - protein, 33 aa, Homo sapiens
ESINGFEAEFNKSQTSFHLRKPSVHISDTAEYF

SEQ ID NO: 1249 - DNA, 343 bp, Homo sapiens
  source 1..343: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagccaaacccggacaggcaatcagccccagcattttggtgatgggactcgactctccatcctag

SEQ ID NO: 1250 - protein, 114 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSQTRTGNQPQHFGDGTRLSIL

SEQ ID NO: 1251 - protein, 15 aa, Homo sapiens
CASSQTRTGNQPQHF

SEQ ID NO: 1252 - DNA, 337 bp, Homo sapiens
  source 1..337: mol_type = other DNA
ggagagaatgtggagcagcatccttcaaccctgagtgtccaggagggagacagcgctgttatcaagtgtacttattcagacagtgcctcaaactacttcccttggtataagcaagaacttggaaaaggacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtactggtgctacaaacaagctcatctttggaactggcactctgcttgctgtccagccaa

SEQ ID NO: 1253 - protein, 112 aa, Homo sapiens
GENVEQHPSTLSVQEGDSAVIKCTYSDSASNYFPWYKQELGKGPQLIIDIRSNVGEKKDQRIAVTLNKTAKHFSLHITETQPEDSAVYFCAASTGATNKLIFGTGTLLAVQP

SEQ ID NO: 1254 - protein, 13 aa, Homo sapiens
CAASTGATNKLIF

SEQ ID NO: 1255 - DNA, 349 bp, Homo sapiens
  source 1..349: mol_type = other DNA
gaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagcttagttggacagggggcctacaatgagcagttcttcgggccagggacacggctcaccgtgctag

SEQ ID NO: 1256 - protein, 116 aa, Homo sapiens
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLVGQGAYNEQFFGPGTRLTVL

SEQ ID NO: 1257 - protein, 16 aa, Homo sapiens
CASSLVGQGAYNEQFF

SEQ ID NO: 1258 - DNA, 340 bp, Homo sapiens
  source 1..340: mol_type = other DNA
cggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaaccctcatggctctagcaacacaggcaaactaatctttgggcaagggacaactttacaagtaaaaccag

SEQ ID NO: 1259 - protein, 113 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVNPHGSSNTGKLIFGQGTTLQVKP

SEQ ID NO: 1260 - protein, 16 aa, Homo sapiens
CVVNPHGSSNTGKLIF

SEQ ID NO: 1261 - protein, 10 aa, Homo sapiens
GQGTTLQVKP

SEQ ID NO: 1262 - DNA, 352 bp, Homo sapiens
  source 1..352: mol_type = other DNA
aatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttctgtgccagcagcttcatcggttggggcttagcaccccggggggcccagtacttcgggccaggcacgcggctcctggtgctcg

SEQ ID NO: 1263 - protein, 117 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSFIGWGLAPRGAQYFGPGTRLLVL

SEQ ID NO: 1264 - protein, 18 aa, Homo sapiens
CASSFIGWGLAPRGAQYF

SEQ ID NO: 1265 - DNA, 349 bp, Homo sapiens
  source 1..349: mol_type = other DNA
gctcagtcagtggctcagccggaagatcaggtcaacgttgctgaagggaatcctctgactgtgaaatgcacctattcagtctctggaaacccttatcttttttggtatgttcaataccccaaccgaggcctccagttccttctgaaatacatcacaggggataacctggttaaaggcagctatggctttgaagctgaatttaacaagagccaaacctccttccacctgaagaaaccatctgcccttgtgagcgactccgctttgtacttctgtgctgtgagattcgacagaggctcaaccctggggaggctatactttggaagaggaactcagttgactgtctggcctg

SEQ ID NO: 1266 - protein, 116 aa, Homo sapiens
AQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRFDRGSTLGRLYFGRGTQLTVWP

SEQ ID NO: 1267 - protein, 16 aa, Homo sapiens
CAVRFDRGSTLGRLYF

SEQ ID NO: 1268 - DNA, 331 bp, Homo sapiens
  source 1..331: mol_type = other DNA
gacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagcctgagagaaaaactgttttttggcagtggaacccagctctctgtcttgg

SEQ ID NO: 1269 - protein, 110 aa, Homo sapiens
DTEVTQTPKHLVMGMTNKKSLKCEQHMGHRAMYWYKQKAKKPPELMFVYSYEKLSINESVPSRFSPECPNSSLLNLHLHALQPEDSALYLCASSLREKLFFGSGTQLSVL

SEQ ID NO: 1270 - protein, 11 aa, Homo sapiens
CASSLREKLFF

SEQ ID NO: 1271 - DNA, 334 bp, Homo sapiens
  source 1..334: mol_type = other DNA
ggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagaaaatgctggcaacaaccgtaagctgatttggggattgggaacaagcctggcagtaaatccga

SEQ ID NO: 1272 - protein, 111 aa, Homo sapiens
GEDVEQSLFLSVREGDSSVINCTYTDSSSTYLYWYKQEPGAGLQLLTYIFSNMDMKQDQRLTVLLNKKDKHLSLRIADTQTGDSAIYFCAENAGNNRKLIWGLGTSLAVNP

SEQ ID NO: 1273 - protein, 13 aa, Homo sapiens
CAENAGNNRKLIW

SEQ ID NO: 1274 - DNA, 346 bp, Homo sapiens
  source 1..346: mol_type = other DNA
gatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtcggggacagggtgtctcctacgagcagtacttcgggccgggcaccaggctcacggtcacag

SEQ ID NO: 1275 - protein, 115 aa, Homo sapiens
DAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSRGQGVSYEQYFGPGTRLTVT

SEQ ID NO: 1276 - protein, 15 aa, Homo sapiens
CASSRGQGVSYEQYF

SEQ ID NO: 1277 - DNA, 334 bp, Homo sapiens
  source 1..334: mol_type = other DNA
cggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgagggctaactttggaaatgagaaattaacctttgggactggaacaagactcaccatcataccca

SEQ ID NO: 1278 - protein, 111 aa, Homo sapiens
RKEVEQDPGPFNVPEGATVAFNCTYSNSASQSFFWYRQDCRKEPKLLMSVYSSGNEDGRFTAQLNRASQYISLLIRDSKLSDSATYLCVVRANFGNEKLTFGTGTRLTIIP

SEQ ID NO: 1279 - protein, 14 aa, Homo sapiens
CVVRANFGNEKLTF

SEQ ID NO: 1280 - DNA, 343 bp, Homo sapiens
  source 1..343: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagtggacagacagatacgcagtattttggcccaggcacccggctgacagtgctcg

SEQ ID NO: 1281 - protein, 114 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSASGQTDTQYFGPGTRLTVL

SEQ ID NO: 1282 - protein, 12 aa, Homo sapiens
CSASGQTDTQYF

SEQ ID NO: 1283 - DNA, 337 bp, Homo sapiens
  source 1..337: mol_type = other DNA
cagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgagggcttatactggaggcttcaaaactatctttggagcaggaacaagactatttgttaaagcaa

SEQ ID NO: 1284 - protein, 112 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMRAYTGGFKTIFGAGTRLFVKA

SEQ ID NO: 1285 - protein, 14 aa, Homo sapiens
CAMRAYTGGFKTIF

SEQ ID NO: 1286 - DNA, 346 bp, Homo sapiens
  source 1..346: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaggccgaggggcaacactgaagctttctttggacaaggcaccagactcacagttgtag

SEQ ID NO: 1287 - protein, 115 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARPRGNTEAFFGQGTRLTVV

SEQ ID NO: 1288 - protein, 13 aa, Homo sapiens
CSARPRGNTEAFF

SEQ ID NO: 1289 - DNA, 337 bp, Homo sapiens
  source 1..337: mol_type = other DNA
ggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtgcggctgaattatggtggtgctacaaacaagctcatctttggaactggcactctgcttgctgtccagccaa

SEQ ID NO: 1290 - protein, 112 aa, Homo sapiens
GIQVEQSPPDLILQEGANSTLRCNFSDSVNNLQWFHQNPWGQLINLFYIPSGTKQNGRLSATTVATERYSLLYISSSQTTDSGVYFCAVRLNYGGATNKLIFGTGTLLAVQP

SEQ ID NO: 1291 - protein, 16 aa, Homo sapiens
CAVRLNYGGATNKLIF

SEQ ID NO: 1292 - DNA, 349 bp, Homo sapiens
  source 1..349: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagaccgcctcagggggacactgaagctttctttggacaaggcaccagactcacagttgtag

SEQ ID NO: 1293 - protein, 116 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARPPQGDTEAFFGQGTRLTVV

SEQ ID NO: 1294 - protein, 14 aa, Homo sapiens
CSARPPQGDTEAFF

SEQ ID NO: 1295 - DNA, 328 bp, Homo sapiens
  source 1..328: mol_type = other DNA
ggacaaaacattgaccagcccactgagatgacagctacggaaggtgccattgtccagatcaactgcacgtaccagacatctgggttcaacgggctgttctggtaccagcaacatgctggcgaagcacccacatttctgtcttacaatgttctggatggtttggaggagaaaggtcgtttttcttcattccttagtcggtctaaagggtacagttacctccttttgaaggagctccagatgaaagactctgcctcttacctctgtgctgttatggatagcaactatcagttaatctggggcgctgggaccaagctaattataaagccag

SEQ ID NO: 1296 - protein, 109 aa, Homo sapiens
GQNIDQPTEMTATEGAIVQINCTYQTSGFNGLFWYQQHAGEAPTFLSYNVLDGLEEKGRFSSFLSRSKGYSYLLLKELQMKDSASYLCAVMDSNYQLIWGAGTKLIIKP

SEQ ID NO: 1297 - protein, 12 aa, Homo sapiens
CAVMDSNYQLIW

SEQ ID NO: 1298 - DNA, 334 bp, Homo sapiens
  source 1..334: mol_type = other DNA
gatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccactcagggtagcaatcagccccagcattttggtgatgggactcgactctccatcctag

SEQ ID NO: 1299 - protein, 111 aa, Homo sapiens
DADVTQTPRNRITKTGKRIMLECSQTKGHDRMYWYRQDPGLGLRLIYYSFDVKDINKGEISDGYSVSRQAQAKFSLSLESAIPNQTALYFCATQGSNQPQHFGDGTRLSIL

SEQ ID NO: 1300 - protein, 12 aa, Homo sapiens
CATQGSNQPQHF

SEQ ID NO: 1301 - DNA, 331 bp, Homo sapiens
  source 1..331: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaacgctgcaggcaacaagctaacttttggaggaggaaccagggtgctagttaaaccaa

SEQ ID NO: 1302 - protein, 110 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNAAGNKLTFGGGTRVLVKP

SEQ ID NO: 1303 - protein, 12 aa, Homo sapiens
CAVNAAGNKLTF

SEQ ID NO: 1304 - protein, 17 aa, Homo sapiens
FFWYRQYSGKSPELIMS

SEQ ID NO: 1305 - DNA, 334 bp, Homo sapiens
  source 1..334: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccacttcggtttattacaatgagcagttcttcgggccagggacacggctcaccgtgctag

SEQ ID NO: 1306 - protein, 111 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCATSVYYNEQFFGPGTRLTVL

SEQ ID NO: 1307 - protein, 12 aa, Homo sapiens
CATSVYYNEQFF

SEQ ID NO: 1308 - DNA, 331 bp, Homo sapiens
  source 1..331: mol_type = other DNA
cagaaggaggtggagcaggatcctggaccactcagtgttccagagggagccattgtttctctcaactgcacttacagcaacagtgcttttcaatacttcatgtggtacagacagtattccagaaaaggccctgagttgctgatgtacacatactccagtggtaacaaagaagatggaaggtttacagcacaggtcgataaatccagcaagtatatctccttgttcatcagagactcacagcccagtgattcagccacctacctctgtgcaatgaccgataacaccgacaagctcatctttgggactgggaccagattacaagtctttccaa

SEQ ID NO: 1309 - protein, 110 aa, Homo sapiens
QKEVEQDPGPLSVPEGAIVSLNCTYSNSAFQYFMWYRQYSRKGPELLMYTYSSGNKEDGRFTAQVDKSSKYISLFIRDSQPSDSATYLCAMTDNTDKLIFGTGTRLQVFP

SEQ ID NO: 1310 - protein, 12 aa, Homo sapiens
CAMTDNTDKLIF

SEQ ID NO: 1311 - DNA, 349 bp, Homo sapiens
  source 1..349: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctcgactgtgggggctaacctacgagcagtacttcgggccgggcaccaggctcacggtcacag

SEQ ID NO: 1312 - protein, 116 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARLWGLTYEQYFGPGTRLTVT

SEQ ID NO: 1313 - protein, 14 aa, Homo sapiens
CSARLWGLTYEQYF

SEQ ID NO: 1314 - DNA, 334 bp, Homo sapiens
  source 1..334: mol_type = other DNA
cagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgaacacaccatatggaaacaaactggtctttggcgcaggaaccattctgagagtcaagtcct

SEQ ID NO: 1315 - protein, 111 aa, Homo sapiens
QKEVEQNSGPLSVPEGAIASLNCTYSDRGSQSFFWYRQYSGKSPELIMSIYSNGDKEDGRFTAQLNKASQYVSLLIRDSQPSDSATYLCAVNTPYGNKLVFGAGTILRVKS

SEQ ID NO: 1316 - protein, 13 aa, Homo sapiens
CAVNTPYGNKLVF

SEQ ID NO: 1317 - DNA, 349 bp, Homo sapiens
  source 1..349: mol_type = other DNA
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagatcggtgggcaggctatggctacaccttcggttcggggaccaggttaaccgttgtag

SEQ ID NO: 1318 - protein, 116 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDRWAGYGYTFGSGTRLTVV

SEQ ID NO: 1319 - protein, 14 aa, Homo sapiens
CSARDRWAGYGYTF

SEQ ID NO: 1320 - DNA, 343 bp, Homo sapiens
  source 1..343: mol_type = other DNA
gctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcccaatacagtaacaataacaatgacatgcgctttggagcagggaccagactgacagtaaaaccaa

SEQ ID NO: 1321 - protein, 114 aa, Homo sapiens
AQTVTQSQPEMSVQEAETVTLSCTYDTSESDYYLFWYKQPPSRQMILVIRQEAYKQQNATENRFSVNFQKAAKSFSLKISDSQLGDAAMYFCAQYSNNNNDMRFGAGTRLTVKP

SEQ ID NO: 1322 - protein, 13 aa, Homo sapiens
CAQYSNNNNDMRF

SEQ ID NO: 1323 - DNA, 337 bp, Homo sapiens
  source 1..337: mol_type = other DNA
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagcctacaggggactagaggctacaccttcggttcggggaccaggttaaccgttgtag

SEQ ID NO: 1324 - protein, 112 aa, Homo sapiens
DGGITQSPKYLFRKEGQNVTLSCEQNLNHDAMYWYRQDPGQGLRLIYYSQIVNDFQKGDIAEGYSVSREKKESFPLTVTSAQKNPTAFYLCASSLQGTRGYTFGSGTRLTVV

SEQ ID NO: 1325 - protein, 13 aa, Homo sapiens
CASSLQGTRGYTF

SEQ ID NO: 1326 - DNA, 322 bp, Homo sapiens
  source 1..322: mol_type = other DNA
gcccagagagtgactcagcccgagaagctcctctctgtctttaaaggggccccagtggagctgaagtgcaactattcctattctgggagtcctgaactcttctggtatgtccagtactccagacaacgcctccagttactcttgagacacatctctagagagagcatcaaaggcttcactgctgaccttaacaaaggcgagacatctttccacctgaagaaaccatttgctcaagaggaagactcagccatgtattactgtgctctcctatatggaaacaagctggtctttggcgcaggaaccattctgagagtcaagtcct

SEQ ID NO: 1327 - protein, 107 aa, Homo sapiens
AQRVTQPEKLLSVFKGAPVELKCNYSYSGSPELFWYVQYSRQRLQLLLRHISRESIKGFTADLNKGETSFHLKKPFAQEEDSAMYYCALLYGNKLVFGAGTILRVKS

SEQ ID NO: 1328 - protein, 11 aa, Homo sapiens
CALLYGNKLVF

SEQ ID NO: 1329 - DNA, 337 bp, Homo sapiens
  source 1..337: mol_type = other DNA
gaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagactcggccctgtatctctgtgccagcagccaagtgtccaccggggagctgttttttggagaaggctctaggctgaccgtactgg

SEQ ID NO: 1330 - protein, 112 aa, Homo sapiens
ETGVTQTPRHLVMGMTNKKSLKCEQHLGHNAMYWYKQSAKKPLELMFVYNFKEQTENNSVPSRFSPECPNSSHLFLHLHTLQPEDSALYLCASSQVSTGELFFGEGSRLTVL

SEQ ID NO: 1331 - protein, 13 aa, Homo sapiens
CASSQVSTGELFF

SEQ ID NO: 1332 - DNA, 319 bp, Homo sapiens
  source 1..319: mol_type = other DNA
aaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgttgtttctggtggctacaataagctgatttttggagcagggaccaggctggctgtacacccat

SEQ ID NO: 1333 - protein, 106 aa, Homo sapiens
KDQVFQPSTVASSEGAVVEIFCNHSVSNAYNFFWYLHFPGCAPRLLVKGSKPSQQGRYNMTYERFSSSLLILQVREADAAVYYCAVVSGGYNKLIFGAGTRLAVHP

SEQ ID NO: 1334 - protein, 13 aa, Homo sapiens
CAVVSGGYNKLIF

SEQ ID NO: 1335 - DNA, 337 bp, Homo sapiens
  source 1..337: mol_type = other DNA
gaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccaacctgcaaagcttgaggactcggccgtgtatctctgtgccagcagcctagggtacgatacgcagtattttggcccaggcacccggctgacagtgctcg

SEQ ID NO: 1336 - protein, 112 aa, Homo sapiens
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLGYDTQYFGPGTRLTVL

SEQ ID NO: 1337 - protein, 12 aa, Homo sapiens
CASSLGYDTQYF

SEQ ID NO: 1338 - DNA, 343 bp, Homo sapiens
  source 1..343: mol_type = other DNA
ggacaacaggtaatgcaaattcctcagtaccagcatgtacaagaaggagaggacttcaccacgtactgcaattcctcaactactttaagcaatatacagtggtataagcaaaggcctggtggacatcccgtttttttgatacagttagtgaagagtggagaagtgaagaagcagaaaagactgacatttcagtttggagaagcaaaaaagaacagctccctgcacatcacagccacccagactacagatgtaggaacctacttctgtgcagggcgcgggaactctggggctgggagttaccaactcactttcgggaaggggaccaaactctcggtcataccaa

SEQ ID NO: 1339 - protein, 114 aa, Homo sapiens
GQQVMQIPQYQHVQEGEDFTTYCNSSTTLSNIQWYKQRPGGHPVFLIQLVKSGEVKKQKRLTFQFGEAKKNSSLHITATQTTDVGTYFCAGRGNSGAGSYQLTFGKGTKLSVIP

SEQ ID NO: 1340 - protein, 16 aa, Homo sapiens
CAGRGNSGAGSYQLTF

SEQ ID NO: 1341 - DNA, 337 bp, Homo sapiens
  source 1..337: mol_type = other DNA
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagtcccaggggctacaatgagcagttcttcgggccagggacacggctcaccgtgctag

SEQ ID NO: 1342 - protein, 112 aa, Homo sapiens
KAGVTQTPRYLIKTRGQQVTLSCSPISGHRSVSWYQQTPGQGLQFLFEYFSETQRNKGNFPGRFSGRQFSNSRSEMNVSTLELGDSALYLCASSPRGYNEQFFGPGTRLTVL

SEQ ID NO: 1343 - protein, 13 aa, Homo sapiens
CASSPRGYNEQFF

SEQ ID NO: 1344 - DNA, 343 bp, Homo sapiens
  source 1..343: mol_type = other DNA
aaacaggaggtgacgcagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgccgcatactctggggctgggagttaccaactcactttcgggaaggggaccaaactctcggtcataccaa

SEQ ID NO: 1345 - protein, 114 aa, Homo sapiens
KQEVTQIPAALSVPEGENLVLNCSFTDSAIYNLQWFRQDPGKGLTSLLLIQSSQREQTSGRLNASLDKSSGRSTLYIAASQPGDSATYLCAAAYSGAGSYQLTFGKGTKLSVIP

SEQ ID NO: 1346 - protein, 15 aa, Homo sapiens
CAAAYSGAGSYQLTF

SEQ ID NO: 1347 - DNA, 337 bp, Homo sapiens
  source 1..337: mol_type = other DNA
agtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgcccccctaaacgactacaagctcagctttggagccggaaccacagtaactgtaagagcaa

SEQ ID NO: 1348 - protein, 112 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDAPLNDYKLSFGAGTTVTVRA

SEQ ID NO: 1349 - protein, 14 aa, Homo sapiens
CATDAPLNDYKLSF

SEQ ID NO: 1350 - protein, 177 aa, Homo sapiens
EDLKNVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 1351 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 1352 - protein, 5 aa, synthetic construct
GSGGS

SEQ ID NO: 1353 - protein, 4 aa, synthetic construct
GGGS

SEQ ID NO: 1354 - protein, 4 aa, synthetic construct
GGSG

SEQ ID NO: 1355 - protein, 5 aa, synthetic construct
GGSGG

SEQ ID NO: 1356 - protein, 5 aa, synthetic construct
GSGSG

SEQ ID NO: 1357 - protein, 5 aa, synthetic construct
GSGGG

SEQ ID NO: 1358 - protein, 5 aa, synthetic construct
GGGSG

SEQ ID NO: 1359 - protein, 5 aa, synthetic construct
GSSSG

(19) United States
(12) Patent Application Publication
(10) Pub. No.: US 2025/0242026 A1
(43) Pub. Date: Jul. 31, 2025

(54) METHODS AND COMPOSITIONS FOR TREATING GLIOBLASTOMA

(71) Applicant: THE REGENTS OF THE UNIVERSITY OF CALIFORNIA, Oakland, CA (US)

(72) Inventors: Robert PRINS, Los Angeles, CA (US); Alexander H. LEE, Los Angeles, CA (US); Lu SUN, Los Angeles, CA (US); David NATHANSON, Los Angeles, CA (US)

(73) Assignee: THE REGENTS OF THE UNIVERSITY OF CALIFORNIA, Oakland, CA (US)

(21) Appl. No.: 18/704,177

(22) PCT Filed: Oct. 25, 2022
(86) PCT No.: PCT/US2022/078623

§ 371 Date: Apr. 24, 2024

Related U.S. Application Data
(60) Provisional application No. 63/271,514, filed on Oct. 25, 2021.

(51) Int. Cl. A61K 40/42; A61K 40/11; A61K 40/31; A61K 40/32; C07K 14/725; C07K 16/28

(52) U.S. Cl. CPC A61K 40/4211; A61K 40/11; A61K 40/31; A61K 40/32; C07K 14/7051; C07K 16/2803

(57) ABSTRACT

This disclosure provides for engineered T cell Receptors (TCRs), cells comprising the TCRs, and methods of making and using TCRs. The current disclosure relates to TCRs that specifically recognize cancer antigens. Also provided are compositions comprising the cells, nucleic acids, or engineered TCRs of the disclosure, methods of making the cells, and methods of using the embodiments of the disclosure for therapeutic treatments.

This application claims priority of U.S. Provisional Patent Application No. 63/271,514, filed Oct. 25, 2021, which is hereby incorporated by reference in its entirety.

### STATEMENT OF GOVERNMENT SUPPORT

This invention was made with government support under CA222695, and CA211015 awarded by the National Institutes of Health. The government has certain rights in the invention.

The application contains a Sequence Listing prepared in compliance with ST.26 format and is hereby incorporated by reference in its entirety. Said Sequence Listing, created on Oct. 19, 2022 is named UCLAP0149WO.xml and is 1,314,072 bytes in size.

### BACKGROUND OF THE INVENTION

### II. Field of the Invention

This invention relates to the field of cancer therapy.

### III. Background

CAR-T therapies have shown incredible efficacy in several liquid tumor indications and have provided the framework for the manufacturing of new adoptive cell therapies (ACT). Yet, despite many efforts, the modality is still experiencing difficulties in solid tumors. The lack of public, tumor-specific targets and the suppressive state of the tumor microenvironment (TME) are two significant challenges in overcoming the barrier of effective CAR-T therapies in solid tumors. A promising strategy for solid tumors is to identify a patient's own tumor reactive T cell receptors (TCRs) and to then engineer these into healthy cells capable of mediating a response once infused back into the patient (TCR-T). Such a therapy would be polyclonal, which can specifically and broadly recognize the cancer as well as possess a phenotype capable of eliciting a response despite a suppressive TME. While techniques exist to identify personalized TCRs and engineer them into cells with a desirable phenotype, significant innovation is needed to reduce the time and expense to move towards a clinically feasible product.

Powerful therapies such as the personalized TCR-T described above are needed for the treatment of moderately immunogenic and heterogeneous cancer indications, such as glioblastoma (GBM). Despite the promise of available immunotherapies, most clinical trials for patients diagnosed with GBM have produced disappointing results to date.

### SUMMARY OF THE INVENTION

This disclosure provides for polypeptides, engineered T cell Receptors (TCRs), cells comprising the TCRs, and methods of making and using the TCRs. Accordingly, aspects of the disclosure relate to a polypeptide an antigen binding variable region comprising a complementarity determining region (CDR) 3 comprising the amino acid sequence of a CDR3-b identified from a T-cell receptor (TCR) clone in Table 1 or an amino acid sequence with at least 80% sequence identity to the amino acid sequence of a CDR3-b from a TCR clone in Table 1. Also provided are engineered TCRs comprising a TCR-b polypeptide and a TCR-a polypeptide, wherein the TCR-b polypeptide comprises a CDR3 comprising the amino acid sequence of a CDR3-b identified from a TCR clone in Table 1 or an amino acid sequence with at least 80% sequence identity to the amino acid sequence of a CDR3-b from a TCR clone identified in Table 1 and the TCR-a polypeptide comprises a CDR3 comprising the amino acid sequence of a CDR3-a identified from the corresponding TCR clone in Table 1 or an amino acid sequence with at least 80% sequence identity to the amino acid sequence of a CDR3-a from the corresponding TCR clone identified in Table 1. Further aspects relate to a fusion protein comprising a polypeptide or TCR of the disclosure and a CD3 binding region. Also described are nucleic acids encoding the polypeptides, TCRs or fusion proteins of the disclosure, expression vectors comprising nucleic acids of the disclosure, and cells comprising the polypeptides, TCRs, fusion proteins, nucleic acids, and/or expression vectors of the disclosure. Further aspects relate to compositions comprise the polypeptides, TCRs, fusion proteins, nucleic acids, expression vectors, and/or cells of the disclosure. Aspects of the disclosure provide for a method of making an engineered cell comprising transferring the nucleic acid(s) or vector(s) of the disclosure into the cell. Methods of the disclosure also include a method of making a polypeptide comprising expressing a nucleic acid or vector of the disclosure in a cell. The method may further comprise isolating the expressed polypeptide. Further aspects relate to a method for treating or preventing cancer in a subject comprising administering a composition of the disclosure to a subject in need thereof. Yet further aspects describe a method of stimulating an immune response in a subject, the method comprising administering a composition of the disclosure to a subject in need thereof.

Methods also include methods of reducing tumor burden; methods of lysing a cancer cell; methods of killing tumor/cancerous cells; methods of increasing overall survival; methods of reducing the risk of getting cancer or of getting a tumor; methods of increasing recurrent free survival; methods of preventing cancer; and/or methods of reducing, eliminating, or decreasing the spread or metastasis of cancer, the method comprising administering a polypeptide, composition, cell, nucleic acid, or engineered TCR to a subject in need thereof. Methods also include methods of reducing tumor burden; methods of lysing a cancer cell; methods of killing tumor/cancerous cells; methods of increasing overall survival; methods of preventing or reducing the risk of recurrence of cancer; methods of promoting cancer remission; methods of increasing susceptibility to other or additional cancer treatments; methods of reducing the risk of getting cancer or of getting a tumor; methods of increasing recurrent free survival; methods of preventing cancer; and/or methods of reducing, eliminating, or decreasing the spread or metastasis of cancer, the method comprising administering a polypeptide, composition, cell, nucleic acid, or engineered TCR to a subject in need thereof, as set forth herein.

The variable region may comprise a CDR1, CDR2, and/or CDR3. The variable region may comprise a CDR1 with the amino acid sequence of the CDR1-b from the corresponding TCR clone in Table 1 or an amino acid sequence with at least 80% sequence identity to the CDR1-b from the corresponding TCR clone in Table 1. In some aspects, the variable region comprises a CDR1 with the amino acid sequence of the CDR1-b from the corresponding TCR clone in Table 1 or an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the CDR1-b from the corresponding TCR clone in Table 1. The variable region may comprise a CDR2 with the amino acid sequence of the CDR2-b from the corresponding TCR clone in Table 1 or an amino acid sequence with at least 80% sequence identity to the CDR2-b from the corresponding TCR clone in Table 1. In some aspects, the variable region comprises a CDR2 with the amino acid sequence of the CDR2-b from the corresponding TCR clone in Table 1 or an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the CDR2-b from the corresponding TCR clone in Table 1. The polypeptide may comprise a CDR1, CDR2, and CDR3 with an amino acid sequence of a CDR1-b, CDR2-b, and CDR3-b from the corresponding TCR clone in Table 1. In some aspects, the variable region comprises the amino acid sequence of the variable-b from the corresponding TCR clone in Table 1 or an amino acid sequence with at least 80% sequence identity to the variable-b from the corresponding TCR clone in Table 1. In some aspects, the variable region comprises the amino acid sequence of the variable-b from the corresponding TCR clone in Table 1 or an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the variable-b from the corresponding TCR clone in Table 1. The polypeptide may comprise a T cell receptor beta (TCR-b) variable region from the corresponding TCR clone in Table 1. In some aspects, the polypeptide comprises a TCR-beta constant region. An exemplary TCR-beta constant region is provided in SEQ ID NO:1350: EDLKNVFPPEVAVFEPSEAEISHTQKATLVCLATGFFPDHVELSWWVNGKEVHSGVS TDPQPLKEQPALNDSRYCLSSRLRVSATFWQNPRNHFRCQVQFYGLSENDEWTQDR AKPVTQIVSAEAWGRADCGFTSVSYQQGVLSATILYEILLGKATLYAVLVSALVLMA MVKRKDF (SEQ ID NO:1350).

The variable region may comprise a CDR1 with the amino acid sequence of the CDR1-a from the corresponding TCR clone in Table 1 or an amino acid sequence with at least 80% sequence identity to the CDR1-a from the corresponding TCR clone in Table 1. In some aspects, the variable region comprises a CDR1 with the amino acid sequence of the CDR1-a from the corresponding TCR clone in Table 1 or an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the CDR1-a from the corresponding TCR clone in Table 1. The variable region may comprise a CDR2 with the amino acid sequence of the CDR2-a from the corresponding TCR clone in Table 1 or an amino acid sequence with at least 80% sequence identity to the CDR2-a from the corresponding TCR clone in Table 1. In some aspects, the variable region comprises a CDR2 with the amino acid sequence of the CDR2-a from the corresponding TCR clone in Table 1 or an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the CDR2-a from the corresponding TCR clone in Table 1. The polypeptide may comprise a CDR1, CDR2, and CDR3 with an amino acid sequence of a CDR1-a, CDR2-a, and CDR3-a from the corresponding TCR clone in Table 1. In some aspects, the variable region comprises the amino acid sequence of the variable-a from the corresponding TCR clone in Table 1 or an amino acid sequence with at least 80% sequence identity to the variable-a from the corresponding TCR clone in Table 1. In some aspects, the variable region comprises the amino acid sequence of the variable-a from the corresponding TCR clone in Table 1 or an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the variable-a from the corresponding TCR clone in Table 1. The polypeptide may comprise a T cell receptor beta (TCR-a) variable region from the corresponding TCR clone in Table 1. In some aspects, the polypeptide comprises a TCR-alpha constant region. An exemplary TCR-alpha constant region is provided in SEQ ID NO:1351: IQNPDPAVYQLRDSKSSDKSVCLFTDFDSQTNVSQSKDSDVYITDKTVLDMRSMDFK SNSAVAWSNKSDFACANAFNNSIIPEDTFFPSPESSCDVKLVEKSFETDTNLNFQNLS VIGFRILLLKVAGFNLLMTLRLWSS (SEQ ID NO:1351).

In some aspects, the polypeptide may comprise or further comprise a signal peptide.

The TCR may comprise a TCR-b polypeptide comprising a variable region comprising CDR1, CDR2, and CDR3 and a TCR-a polypeptide comprising a variable region comprising CDR1, CDR2, and CDR3. The TCR-b polypeptide may comprise a CDR1 comprising the amino acid sequence of a CDR1-b of the corresponding TCR clone in Table 1 or an amino acid sequence with at least 80% sequence identity to a CDR1-b of the corresponding TCR clone in Table 1 and/or the TCR-a polypeptide may comprise a CDR1-a of the corresponding TCR clone in Table 1 or an amino acid sequence with at least 80% sequence identity to a CDR1-a of the corresponding TCR clone in Table 1. The TCR-b polypeptide may comprise a CDR1 comprising an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a CDR1-b of the corresponding TCR clone in Table 1 and/or the TCR-a polypeptide may comprise a CDR1 comprising an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a CDR1-a of the corresponding TCR clone in Table 1. The TCR-b polypeptide may comprise a CDR2 with the amino acid sequence of the CDR2-b from the corresponding TCR clone in Table 1 or an amino acid sequence with at least 80% sequence identity to the CDR2-b from the corresponding TCR clone in Table 1 and the TCR-a polypeptide may comprise a CDR2 with the amino acid sequence of the CDR2-b from the corresponding TCR clone in Table 1 or an amino acid sequence with at least 80% sequence identity to the CDR2-b from the corresponding TCR clone in Table 1. The TCR-b polypeptide may comprise a CDR2 with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the CDR2-b from the corresponding TCR clone in Table 1 and the TCR-a polypeptide may comprise a CDR2 with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the CDR2-b from the corresponding TCR clone in Table 1. The CDR1, CDR2, and CDR3 of the TCR-b polypeptide may comprise the amino acid sequence of a CDR1-b, CDR2-b, and CDR3-b from a TCR clone of Table 1 and the CDR1, CDR3, and CDR3 of the TCR-a polypeptide comprise the amino acid sequence of the CDR1-a, CDR2-a, and CDR3-a from the corresponding TCR clone in Table 1. The TCR-b polypeptide may comprise an amino acid sequence with at least 70% sequence identity to the variable-b from the corresponding TCR clone in Table 1 and the TCR-a polypeptide may comprise an amino acid sequence with at least 70% sequence identity to the variable-a from the same TCR clone in Table 1. In some aspects, the TCR-b polypeptide comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the variable-b from the corresponding TCR clone in Table 1 and the TCR-a polypeptide comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the variable-a from the same TCR clone in Table 1. The TCR-b polypeptide comprises the amino acid sequence the variable-b from the corresponding TCR clone in Table 1 and the TCR-a polypeptide comprises the amino acid sequence the variable-b from the corresponding TCR clone in Table 1.

In some aspects, the TCR comprises a modification or is chimeric. In some aspects, the TCR-a polypeptide and TCR-b polypeptide are operably linked. The term “operably linked” can refer to a covalent linkage, such as a peptide bond (e.g. the two elements are polypeptides and are on the same polypeptide), or a non-covalent linkage, such as Van der Waals force (e.g. two polypeptides that have a certain degree of specific binding affinity for each other). The TCR-a polypeptide and TCR-b polypeptide are operably linked through a peptide bond. In some aspects, the TCR is a single chain TCR. In some aspects, the TCR-a polypeptide and TCR-b polypeptide are on the same polypeptide and wherein the TCR-b is amino-proximal to the TCR-a. In some aspects, the TCR-a polypeptide and TCR-b polypeptide are on the same polypeptide and wherein the TCR-a is amino-proximal to the TCR-b. In some aspects, the TCR comprises a linker between the TCR-a and TCR-b polypeptide. The linker may comprise glycine and serine residues. The linker may comprise glycine and serine residues. In some aspects, the linker is composed of only glycine and serine residues (a glycine-serine linker). The linker may be a flexible linker. Exemplary flexible linkers include glycine polymers (G)n, glycine-serine polymers (including, for example, (GS)n, (GSGGS-SEQ ID NO:1352)n, (G₄S)n and (GGGS-SEQ ID NO:1353)n, where n is an integer of at least one. In some aspects, n is at least, at most, or exactly 1, 2, 3, 4, 5, 6, 7, 8, 9, or 10 (or any derivable range therein). Glycine-alanine polymers, alanine-serine polymers, and other flexible linkers known in the art and may be used as a linker in the polypeptides of the disclosure. Exemplary linkers can comprise or consist of GGSG (SEQ ID NO:1354), GGSGG (SEQ ID NO:1355), GSGSG (SEQ ID NO:1356), GSGGG (SEQ ID NO:1357), GGGSG (SEQ ID NO:1358), GSSSG (SEQ ID NO:1359), and the like. Further linkers useful in the polypeptides and TCRs of the disclosure are described herein. A first region is carboxy-proximal to a second region when the first region is attached to the carboxy terminus of the second region. There may be further intervening amino acid residues between the first and second regions. Thus, the regions need not be immediately adjacent, unless specifically specified as not having intervening amino acid residues. The term “amino-proximal” is similarly defined in that a first region is amino-proximal to a second region when the first region is attached to the amino terminus of the second region. Similarly, there may be further intervening amino acid residues between the first and second regions unless stated otherwise.

Further aspects relate to a fusion protein comprising a TCR of the disclosure and a CD3 binding region. The CD3 binding region may comprise a CD3-specific fragment antigen binding (Fab), single chain variable fragment (scFv), single domain antibody, or single chain antibody. Exemplary CD3-specific fragment antigen binding (Fab) are known in the art. For example, US20180222981, which is herein incorporated by reference, discloses variable regions that bind specifically to CD3, which may be used in aspects of this disclosure. Anti-CD3 antibodies and variable regions are disclosed in US20180117152, which is also incorporated by reference. The TCR or fusion protein may be conjugated to a detection or therapeutic agent. The agent may comprise a fluorescent molecule, radiative molecule, or toxin. In some aspects, the TCR or fusion protein is conjugated to an agent described herein.

A CDR may also comprise 1, 2, 3, 4, 5, 6, 7, 8, 9, 10, 11, 12, 13, 14, 15, 16, 16, 18, 19, 20, 21, 22, 23, or more contiguous amino acid residues (or any range derivable therein) flanking one or both sides of a particular CDR sequence; therefore, there may be one or more additional amino acids at the N-terminal or C-terminal end of a particular CDR sequence, such as those shown in Table 1. Alternatively, or in combination, a CDR may also be a fragment of a CDR described herein and may lack at least 1, 2, 3, 4, or 5 amino acids from the C-terminal or N-terminal end of a particular CDR sequence.

Nucleic acids of the disclosure include those that encode for CDR regions, variable regions, engineered TCRs, polypeptides, TCR-a polypeptides, TCR-b polypeptides, peptides, polypeptides, and fusion proteins described herein. The nucleic acid may be RNA. The nucleic acid may also be DNA or a cDNA encoding the peptide or polypeptide, or a complement of the peptide or polypeptide. The nucleic acid may comprise a TCR-a (TRA) and TCR-b (TRB) gene. The nucleic acid may be polycistronic. The nucleic acid may also comprise an internal ribosome entry site (IRES) or a P2A linker. The nucleic acid may comprise a cDNA encoding the TCR-a and/or TCR-b genes. The nucleic acid may encode or further encode for a polypeptide comprising a CD3 binding region. In some aspects, the CD3 binding region comprises a CD3-specific fragment antigen binding (Fab), single chain variable fragment (scFv), single domain antibody, or single chain antibody. The vector may comprise both of the TCR-a and TCR-b genes. The vector may comprise a promoter that directs the expression of the nucleic acid.

In some aspects of the disclosure, the cell comprises a stem cell, a progenitor cell, an immune cell, or a natural killer (NK) cell. In some aspects, the cell comprises a hematopoietic stem or progenitor cell, a T cell, a cell differentiated from mesenchymal stem cells (MSCs) or an induced pluripotent stem cell (iPSC). The cell may be isolated or derived from peripheral blood mononuclear cell (PBMCs). In some aspects, the T cell comprises a cytotoxic T lymphocyte (CTL), a CD8+ T cell, a CD4+ T cell, an invariant NK T (iNKT) cell, a gamma-delta T cell, a NKT cell, or a regulatory T cell. In some aspects, the T cell comprises a CD8+ T cell. In some aspects, the T cell is a CD4+ T cell, a Th1, Th2, Th17, Th9, or Tfh T cell, a cytotoxic T cell, a memory T cell, a central memory T cell, or an effector memory T cell. In some aspects, the cell is isolated from a cancer patient. The patient may have a cancer described herein. In some aspects, the patient has glioblastoma multiforme. In some aspects, the patient has a cancer selected from metastatic melanoma, melanoma, renal cell carcinoma, colorectal cancer, hepatocellular carcinoma, non-small cell lung cancer, malignant pleural mesothelioma, Hodgkin's lymphoma, head and neck cancer, urothelial carcinoma, small cell lung cancer, esophageal carcinoma, gastric cancer, cervical cancer, Merkel cell carcinoma, endometrial cancer, squamous cell carcinoma, bladder cancer, breast cancer, or basal cell carcinoma. In some aspects, the cell is isolated from a non-cancerous patient. In some aspects, the cell is isolated from a healthy patient. The cell may be frozen or may have never been frozen. The cell may be in cell culture. In some aspects, the cell lacks endogenous expression of TCR genes. In some aspects, the cell further comprises a chimeric antigen receptor (CAR).

The compositions of the disclosure may be formulated for parenteral administration, intravenous injection, intramuscular injection, inhalation, or subcutaneous injection. In some aspects, the composition is formulated as a vaccine. In some aspects, the composition further comprises an adjuvant. In some aspects, the composition has been determined to be serum-free, mycoplasma-free, endotoxin-free, and/or sterile.

The method may further comprise isolating the expressed polypeptide. In some aspects, the method comprises or further comprises culturing the cell in media, incubating the cell at conditions that allow for the division of the cell, screening the cell, and/or freezing the cell.

In some aspects, the subject has been diagnosed with cancer, such as a cancer described herein. In some aspects, the cancer comprises glioblastoma multiforme. In some aspects the cancer is selected from metastatic melanoma, melanoma, renal cell carcinoma, colorectal cancer, hepatocellular carcinoma, non-small cell lung cancer, malignant pleural mesothelioma, Hodgkin's lymphoma, head and neck cancer, urothelial carcinoma, small cell lung cancer, esophageal carcinoma, gastric cancer, cervical cancer, Merkel cell carcinoma, endometrial cancer, squamous cell carcinoma, bladder cancer, breast cancer, or basal cell carcinoma. In some aspects, the cancer excludes metastatic melanoma, melanoma, renal cell carcinoma, colorectal cancer, hepatocellular carcinoma, non-small cell lung cancer, malignant pleural mesothelioma, Hodgkin's lymphoma, head and neck cancer, urothelial carcinoma, small cell lung cancer, esophageal carcinoma, gastric cancer, cervical cancer, Merkel cell carcinoma, endometrial cancer, squamous cell carcinoma, bladder cancer, breast cancer, or basal cell carcinoma. The subject may be one that has been previously been treated for the cancer. The subject may be one that has been determined to be resistant to the previous treatment. The method may comprise or further comprise the administration of an additional therapy. The cancer may be further defined as a solid tumor. The cancer may be a stage I, II, III, or IV cancer. The cancer may comprise metastatic and/or recurrent cancer. In some aspects, the subject is a human. The subject may also be defined as a mammal. In some aspects, the subject is a non-human primate, rat, mouse, laboratory animal, rabbit, horse, or pig. The cells administered to the subject may be autologous. In some aspects, the cell comprises a non-autologous cell. The cell may also be allogenic or xenogenic.

The compositions of the disclosure may be formulated as a vaccine. The compositions and methods of the disclosure provide for prophylactic therapies to prevent cancer. The compositions and methods of the disclosure provide for therapeutic therapies to treat existing cancers, such as for the treatment of patients with cancer. The composition may comprise or further comprise an adjuvant. Adjuvants are known in the art and include, for example, TLR agonists and aluminum salts.

The polypeptide or engineered TCR may comprise a TCR-b variable region and a TCR-a variable region, wherein the TCR-b variable region comprises a CDR1, CDR2, and CDR3 of SEQ ID NOs:3, 4, and 5, respectively, and the TCR-a variable region comprises a CDR1, CDR2, and CDR3 of SEQ ID NOs:12, 13, and 14, respectively.

The polypeptide or engineered TCR may comprise a TCR-b variable region and a TCR-a variable region, wherein the TCR-b variable region comprises a CDR1, CDR2, and CDR3 of SEQ ID NOs:21, 22, and 23, respectively, and the TCR-a variable region comprises a CDR1, CDR2, and CDR3 of SEQ ID NOs:30, 31, and 32, respectively.

The polypeptide or engineered TCR may comprise a TCR-b variable region and a TCR-a variable region, wherein the TCR-b variable region comprises a CDR1, CDR2, and CDR3 of SEQ ID NOs:39, 40, and 41, respectively, and the TCR-a variable region comprises a CDR1, CDR2, and CDR3 of SEQ ID NOs:48, 49, and 50, respectively.

The polypeptide or engineered TCR may comprise a TCR-b variable region and a TCR-a variable region, wherein the TCR-b variable region comprises a CDR1, CDR2, and CDR3 of SEQ ID NOs:57, 58, and 59, respectively, and the TCR-a variable region comprises a CDR1, CDR2, and CDR3 of SEQ ID NOs:70, 71, and 72, respectively.

The polypeptide or engineered TCR may comprise a TCR-b variable region and a TCR-a variable region, wherein the TCR-b variable region comprises a CDR1, CDR2, and CDR3 of SEQ ID NOs:21, 22, and 66, respectively, and the TCR-a variable region comprises a CDR1, CDR2, and CDR3 of SEQ ID NOs:70, 71, and 72, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 81, respectively, and the TCR-a variable region comprises a CDR1, CDR2, and CDR3 of SEQ ID NOs:88, 89, and 90, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 99, respectively, and the TCR-a variable region comprises a CDR1, CDR2, and CDR3 of SEQ ID NOs:106, 107, and 108, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 115, respectively, and the TCR-a variable region comprises a CDR1, CDR2, and CDR3 of SEQ ID NOs:118, 119, and 120, respectively.

The polypeptide or engineered TCR may comprise a TCR-b variable region and a TCR-a variable region, wherein the TCR-b variable region comprises a CDR1, CDR2, and CDR3 of SEQ ID NOs:127, 128, and 129, respectively, and the TCR-a variable region comprises a CDR1, CDR2, and CDR3 of SEQ ID NOs:135, 136, and 137, respectively.

The polypeptide or engineered TCR may comprise a TCR-b variable region and a TCR-a variable region, wherein the TCR-b variable region comprises a CDR1, CDR2, and CDR3 of SEQ ID NOs:144, 145, and 146, respectively, and the TCR-a variable region comprises a CDR1, CDR2, and CDR3 of SEQ ID NOs:153, 154, and 155, respectively.

The polypeptide or engineered TCR may comprise a TCR-b variable region and a TCR-a variable region, wherein the TCR-b variable region comprises a CDR1, CDR2, and CDR3 of SEQ ID NOs:161, 162, and 163, respectively, and the TCR-a variable region comprises a CDR1, CDR2, and CDR3 of SEQ ID NOs:12, 13, and 177, respectively.

The polypeptide or engineered TCR may comprise a TCR-b variable region and a TCR-a variable region, wherein the TCR-b variable region comprises a CDR1, CDR2, and CDR3 of SEQ ID NOs:169, 170, and 171, respectively, and the TCR-a variable region comprises a CDR1, CDR2, and CDR3 of SEQ ID NOs:12, 13, and 177, respectively.

The polypeptide or engineered TCR may comprise a TCR-b variable region and a TCR-a variable region, wherein the TCR-b variable region comprises a CDR1, CDR2, and CDR3 of SEQ ID NOs:181, 182, and 183, respectively, and the TCR-a variable region comprises a CDR1, CDR2, and CDR3 of SEQ ID NOs:118, 119, and 189, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 193, respectively, and the TCR-a variable region comprises a CDR1, CDR2, and CDR3 of SEQ ID NOs:118, 119, and 196, respectively.

The polypeptide or engineered TCR may comprise a TCR-b variable region and a TCR-a variable region, wherein the TCR-b variable region comprises a CDR1, CDR2, and CDR3 of SEQ ID NOs:199, 200, and 201, respectively, and the TCR-a variable region comprises a CDR1, CDR2, and CDR3 of SEQ ID NOs:207, 208, and 209, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 218, respectively, and the TCR-a variable region comprises a CDR1, CDR2, and CDR3 of SEQ ID NOs:224, 225, and 226, respectively.

The polypeptide or engineered TCR may comprise a TCR-b variable region and a TCR-a variable region, wherein the TCR-b variable region comprises a CDR1, CDR2, and CDR3 of SEQ ID NOs:21, 22, and 233, respectively, and the TCR-a variable region comprises a CDR1, CDR2, and CDR3 of SEQ ID NOs:236, 237, and 238, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 245, respectively, and the TCR-a variable region comprises a CDR1, CDR2, and CDR3 of SEQ ID NOs:118, 119, and 249, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 254, respectively, and the TCR-a variable region comprises a CDR1, CDR2, and CDR3 of SEQ ID NOs:118, 119, and 259, respectively.

The polypeptide or engineered TCR may comprise a TCR-b variable region and a TCR-a variable region, wherein the TCR-b variable region comprises a CDR1, CDR2, and CDR3 of SEQ ID NOs:263, 264, and 265, respectively, and the TCR-a variable region comprises a CDR1, CDR2, and CDR3 of SEQ ID NOs:118, 119, and 271, respectively.

The polypeptide or engineered TCR may comprise a TCR-b variable region and a TCR-a variable region, wherein the TCR-b variable region comprises a CDR1, CDR2, and CDR3 of SEQ ID NOs:275, 276, and 277, respectively, and the TCR-a variable region comprises a CDR1, CDR2, and CDR3 of SEQ ID NOs:286, 287, and 288, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 283, respectively, and the TCR-a variable region comprises a CDR1, CDR2, and CDR3 of SEQ ID NOs:286, 287, and 288, respectively.

The polypeptide or engineered TCR may comprise a TCR-b variable region and a TCR-a variable region, wherein the TCR-b variable region comprises a CDR1, CDR2, and CDR3 of SEQ ID NOs:295, 296, and 297, respectively, and the TCR-a variable region comprises a CDR1, CDR2, and CDR3 of SEQ ID NOs:303, 304, and 305, respectively.

The polypeptide or engineered TCR may comprise a TCR-b variable region and a TCR-a variable region, wherein the TCR-b variable region comprises a CDR1, CDR2, and CDR3 of SEQ ID NOs:21, 22, and 311, respectively, and the TCR-a variable region comprises a CDR1, CDR2, and CDR3 of SEQ ID NOs:314, 315, and 316, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 323, respectively, and the TCR-a variable region comprises a CDR1, CDR2, and CDR3 of SEQ ID NOs:12, 13, and 326, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 323, respectively, and the TCR-a variable region comprises a CDR1, CDR2, and CDR3 of SEQ ID NOs:303, 304, and 338, respectively.

The polypeptide or engineered TCR may comprise a TCR-b variable region and a TCR-a variable region, wherein the TCR-b variable region comprises a CDR1, CDR2, and CDR3 of SEQ ID NOs:331, 332, and 333, respectively, and the TCR-a variable region comprises a CDR1, CDR2, and CDR3 of SEQ ID NOs:303, 304, and 338, respectively.

The polypeptide or engineered TCR may comprise a TCR-b variable region and a TCR-a variable region, wherein the TCR-b variable region comprises a CDR1, CDR2, and CDR3 of SEQ ID NOs:342, 343, and 344, respectively, and the TCR-a variable region comprises a CDR1, CDR2, and CDR3 of SEQ ID NOs:350, 351, and 352, respectively.

The polypeptide or engineered TCR may comprise a TCR-b variable region and a TCR-a variable region, wherein the TCR-b variable region comprises a CDR1, CDR2, and CDR3 of SEQ ID NOs:144, 145, and 359, respectively, and the TCR-a variable region comprises a CDR1, CDR2, and CDR3 of SEQ ID NOs:303, 304, and 362, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 367, respectively, and the TCR-a variable region comprises a CDR1, CDR2, and CDR3 of SEQ ID NOs:207, 208, and 373, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 377, respectively, and the TCR-a variable region comprises a CDR1, CDR2, and CDR3 of SEQ ID NOs:224, 225, and 380, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 385, respectively, and the TCR-a variable region comprises a CDR1, CDR2, and CDR3 of SEQ ID NOs:207, 208, and 391, respectively.

The polypeptide or engineered TCR may comprise a TCR-b variable region and a TCR-a variable region, wherein the TCR-b variable region comprises a CDR1, CDR2, and CDR3 of SEQ ID NOs:395, 396, and 397, respectively, and the TCR-a variable region comprises a CDR1, CDR2, and CDR3 of SEQ ID NOs:403, 404, and 405, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 412, respectively, and the TCR-a variable region comprises a CDR1, CDR2, and CDR3 of SEQ ID NOs:415, 416, and 417, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 423, respectively, and the TCR-a variable region comprises a CDR1, CDR2, and CDR3 of SEQ ID NOs:427, 428, and 429, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 435, respectively, and the TCR-a variable region comprises a CDR1, CDR2, and CDR3 of SEQ ID NOs:88, 89, and 438, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 444, respectively, and the TCR-a variable region comprises a CDR1, CDR2, and CDR3 of SEQ ID NOs:450, 451, and 452, respectively.

The polypeptide or engineered TCR may comprise a TCR-b variable region and a TCR-a variable region, wherein the TCR-b variable region comprises a CDR1, CDR2, and CDR3 of SEQ ID NOs:342, 343, and 459, respectively, and the TCR-a variable region comprises a CDR1, CDR2, and CDR3 of SEQ ID NOs:118, 119, and 465, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 462, respectively, and the TCR-a variable region comprises a CDR1, CDR2, and CDR3 of SEQ ID NOs:118, 119, and 465, respectively.

The polypeptide or engineered TCR may comprise a TCR-b variable region and a TCR-a variable region, wherein the TCR-b variable region comprises a CDR1, CDR2, and CDR3 of SEQ ID NOs:468, 469, and 470, respectively, and the TCR-a variable region comprises a CDR1, CDR2, and CDR3 of SEQ ID NOs:118, 119, and 249, respectively.

The polypeptide or engineered TCR may comprise a TCR-b variable region and a TCR-a variable region, wherein the TCR-b variable region comprises a CDR1, CDR2, and CDR3 of SEQ ID NOs:331, 332, and 476, respectively, and the TCR-a variable region comprises a CDR1, CDR2, and CDR3 of SEQ ID NOs:427, 428, and 479, respectively.

The polypeptide or engineered TCR may comprise a TCR-b variable region and a TCR-a variable region, wherein the TCR-b variable region comprises a CDR1, CDR2, and CDR3 of SEQ ID NOs:169, 170, and 483, respectively, and the TCR-a variable region comprises a CDR1, CDR2, and CDR3 of SEQ ID NOs:486, 287, and 487, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 492, respectively, and the TCR-a variable region comprises a CDR1, CDR2, and CDR3 of SEQ ID NOs:495, 496, and 497, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 503, respectively, and the TCR-a variable region comprises a CDR1, CDR2, and CDR3 of SEQ ID NOs:506, 507, and 508, respectively.

The polypeptide or engineered TCR may comprise a TCR-b variable region and a TCR-a variable region, wherein the TCR-b variable region comprises a CDR1, CDR2, and CDR3 of SEQ ID NOs:514, 515, and 516, respectively, and the TCR-a variable region comprises a CDR1, CDR2, and CDR3 of SEQ ID NOs:153, 154, and 522, respectively.

The polypeptide or engineered TCR may comprise a TCR-b variable region and a TCR-a variable region, wherein the TCR-b variable region comprises a CDR1, CDR2, and CDR3 of SEQ ID NOs:525, 526, and 527, respectively, and the TCR-a variable region comprises a CDR1, CDR2, and CDR3 of SEQ ID NOs:533, 534, and 535, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 542, respectively, and the TCR-a variable region comprises a CDR1, CDR2, and CDR3 of SEQ ID NOs:286, 287, and 545, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 549, respectively, and the TCR-a variable region comprises a CDR1, CDR2, and CDR3 of SEQ ID NOs:552, 553, and 554, respectively.

The polypeptide or engineered TCR may comprise a TCR-b variable region and a TCR-a variable region, wherein the TCR-b variable region comprises a CDR1, CDR2, and CDR3 of SEQ ID NOs:127, 128, and 560, respectively, and the TCR-a variable region comprises a CDR1, CDR2, and CDR3 of SEQ ID NOs:135, 136, and 563, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 566, respectively, and the TCR-a variable region comprises a CDR1, CDR2, and CDR3 of SEQ ID NOs:135, 136, and 569, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 572, respectively, and the TCR-a variable region comprises a CDR1, CDR2, and CDR3 of SEQ ID NOs:575, 576, and 577, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 583, respectively, and the TCR-a variable region comprises a CDR1, CDR2, and CDR3 of SEQ ID NOs:118, 119, and 586, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 590, respectively, and the TCR-a variable region comprises a CDR1, CDR2, and CDR3 of SEQ ID NOs:594, 595, and 596, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 603, respectively, and the TCR-a variable region comprises a CDR1, CDR2, and CDR3 of SEQ ID NOs:575, 576, and 606, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 610, respectively, and the TCR-a variable region comprises a CDR1, CDR2, and CDR3 of SEQ ID NOs:303, 304, and 613, respectively.

The polypeptide or engineered TCR may comprise a TCR-b variable region and a TCR-a variable region, wherein the TCR-b variable region comprises a CDR1, CDR2, and CDR3 of SEQ ID NOs:295, 296, and 616, respectively, and the TCR-a variable region comprises a CDR1, CDR2, and CDR3 of SEQ ID NOs:450, 451, and 619, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 622, respectively, and the TCR-a variable region comprises a CDR1, CDR2, and CDR3 of SEQ ID NOs:303, 304, and 625, respectively.

The polypeptide or engineered TCR may comprise a TCR-b variable region and a TCR-a variable region, wherein the TCR-b variable region comprises a CDR1, CDR2, and CDR3 of SEQ ID NOs:263, 629, and 630, respectively, and the TCR-a variable region comprises a CDR1, CDR2, and CDR3 of SEQ ID NOs:594, 595, and 636, respectively.

The polypeptide or engineered TCR may comprise a TCR-b variable region and a TCR-a variable region, wherein the TCR-b variable region comprises a CDR1, CDR2, and CDR3 of SEQ ID NOs:639, 640, and 641, respectively, and the TCR-a variable region comprises a CDR1, CDR2, and CDR3 of SEQ ID NOs:450, 451, and 647, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 650, respectively, and the TCR-a variable region comprises a CDR1, CDR2, and CDR3 of SEQ ID NOs:314, 315, and 653, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 656, respectively, and the TCR-a variable region comprises a CDR1, CDR2, and CDR3 of SEQ ID NOs:659, 660, and 661, respectively.

The polypeptide or engineered TCR may comprise a TCR-b variable region and a TCR-a variable region, wherein the TCR-b variable region comprises a CDR1, CDR2, and CDR3 of SEQ ID NOs:665, 666, and 667, respectively, and the TCR-a variable region comprises a CDR1, CDR2, and CDR3 of SEQ ID NOs:673, 674, and 675, respectively.

The polypeptide or engineered TCR may comprise a TCR-b variable region and a TCR-a variable region, wherein the TCR-b variable region comprises a CDR1, CDR2, and CDR3 of SEQ ID NOs:199, 200, and 681, respectively, and the TCR-a variable region comprises a CDR1, CDR2, and CDR3 of SEQ ID NOs:427, 428, and 684, respectively.

The polypeptide or engineered TCR may comprise a TCR-b variable region and a TCR-a variable region, wherein the TCR-b variable region comprises a CDR1, CDR2, and CDR3 of SEQ ID NOs:468, 469, and 688, respectively, and the TCR-a variable region comprises a CDR1, CDR2, and CDR3 of SEQ ID NOs:118, 119, and 691, respectively.

The polypeptide or engineered TCR may comprise a TCR-b variable region and a TCR-a variable region, wherein the TCR-b variable region comprises a CDR1, CDR2, and CDR3 of SEQ ID NOs:665, 666, and 694, respectively, and the TCR-a variable region comprises a CDR1, CDR2, and CDR3 of SEQ ID NOs:427, 428, and 697, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 700, respectively, and the TCR-a variable region comprises a CDR1, CDR2, and CDR3 of SEQ ID NOs:594, 595, and 703, respectively.

The polypeptide or engineered TCR may comprise a TCR-b variable region and a TCR-a variable region, wherein the TCR-b variable region comprises a CDR1, CDR2, and CDR3 of SEQ ID NOs:169, 706, and 707, respectively, and the TCR-a variable region comprises a CDR1, CDR2, and CDR3 of SEQ ID NOs:506, 507, and 711, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 714, respectively, and the TCR-a variable region comprises a CDR1, CDR2, and CDR3 of SEQ ID NOs:207, 208, and 717, respectively.

The polypeptide or engineered TCR may comprise a TCR-b variable region and a TCR-a variable region, wherein the TCR-b variable region comprises a CDR1, CDR2, and CDR3 of SEQ ID NOs:331, 332, and 720, respectively, and the TCR-a variable region comprises a CDR1, CDR2, and CDR3 of SEQ ID NOs:106, 107, and 723, respectively.

The polypeptide or engineered TCR may comprise a TCR-b variable region and a TCR-a variable region, wherein the TCR-b variable region comprises a CDR1, CDR2, and CDR3 of SEQ ID NOs:127, 128, and 727, respectively, and the TCR-a variable region comprises a CDR1, CDR2, and CDR3 of SEQ ID NOs:118, 119, and 730, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 733, respectively, and the TCR-a variable region comprises a CDR1, CDR2, and CDR3 of SEQ ID NOs:552, 553, and 736, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 739, respectively, and the TCR-a variable region comprises a CDR1, CDR2, and CDR3 of SEQ ID NOs:207, 208, and 742, respectively.

The polypeptide or engineered TCR may comprise a TCR-b variable region and a TCR-a variable region, wherein the TCR-b variable region comprises a CDR1, CDR2, and CDR3 of SEQ ID NOs:263, 629, and 745, respectively, and the TCR-a variable region comprises a CDR1, CDR2, and CDR3 of SEQ ID NOs:207, 208, and 750, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 753, respectively, and the TCR-a variable region comprises a CDR1, CDR2, and CDR3 of SEQ ID NOs:450, 451, and 756, respectively.

The polypeptide or engineered TCR may comprise a TCR-b variable region and a TCR-a variable region, wherein the TCR-b variable region comprises a CDR1, CDR2, and CDR3 of SEQ ID NOs:331, 332, and 759, respectively, and the TCR-a variable region comprises a CDR1, CDR2, and CDR3 of SEQ ID NOs:762, 763, and 764, respectively.

The polypeptide or engineered TCR may comprise a TCR-b variable region and a TCR-a variable region, wherein the TCR-b variable region comprises a CDR1, CDR2, and CDR3 of SEQ ID NOs:169, 170, and 770, respectively, and the TCR-a variable region comprises a CDR1, CDR2, and CDR3 of SEQ ID NOs:314, 315, and 773, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 777, respectively, and the TCR-a variable region comprises a CDR1, CDR2, and CDR3 of SEQ ID NOs:207, 208, and 780, respectively.

The polypeptide or engineered TCR may comprise a TCR-b variable region and a TCR-a variable region, wherein the TCR-b variable region comprises a CDR1, CDR2, and CDR3 of SEQ ID NOs:665, 666, and 783, respectively, and the TCR-a variable region comprises a CDR1, CDR2, and CDR3 of SEQ ID NOs:118, 119, and 786, respectively.

The polypeptide or engineered TCR may comprise a TCR-b variable region and a TCR-a variable region, wherein the TCR-b variable region comprises a CDR1, CDR2, and CDR3 of SEQ ID NOs:275, 443, and 790, respectively, and the TCR-a variable region comprises a CDR1, CDR2, and CDR3 of SEQ ID NOs:314, 315, and 796, respectively.

The polypeptide or engineered TCR may comprise a TCR-b variable region and a TCR-a variable region, wherein the TCR-b variable region comprises a CDR1, CDR2, and CDR3 of SEQ ID NOs:199, 200, and 799, respectively, and the TCR-a variable region comprises a CDR1, CDR2, and CDR3 of SEQ ID NOs:450, 451, and 802, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 805, respectively, and the TCR-a variable region comprises a CDR1, CDR2, and CDR3 of SEQ ID NOs:12, 13, and 808, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 805, respectively, and the TCR-a variable region comprises a CDR1, CDR2, and CDR3 of SEQ ID NOs:286, 287, and 811, respectively.

The polypeptide or engineered TCR may comprise a TCR-b variable region and a TCR-a variable region, wherein the TCR-b variable region comprises a CDR1, CDR2, and CDR3 of SEQ ID NOs:144, 145, and 815, respectively, and the TCR-a variable region comprises a CDR1, CDR2, and CDR3 of SEQ ID NOs:88, 89, and 819, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 822, respectively, and the TCR-a variable region comprises a CDR1, CDR2, and CDR3 of SEQ ID NOs:403, 404, and 825, respectively.

The polypeptide or engineered TCR may comprise a TCR-b variable region and a TCR-a variable region, wherein the TCR-b variable region comprises a CDR1, CDR2, and CDR3 of SEQ ID NOs:21, 22, and 828, respectively, and the TCR-a variable region comprises a CDR1, CDR2, and CDR3 of SEQ ID NOs:831, 832, and 833, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 839, respectively, and the TCR-a variable region comprises a CDR1, CDR2, and CDR3 of SEQ ID NOs:762, 763, and 842, respectively.

The polypeptide or engineered TCR may comprise a TCR-b variable region and a TCR-a variable region, wherein the TCR-b variable region comprises a CDR1, CDR2, and CDR3 of SEQ ID NOs:845, 846, and 847, respectively, and the TCR-a variable region comprises a CDR1, CDR2, and CDR3 of SEQ ID NOs:30, 31, and 853, respectively.

The polypeptide or engineered TCR may comprise a TCR-b variable region and a TCR-a variable region, wherein the TCR-b variable region comprises a CDR1, CDR2, and CDR3 of SEQ ID NOs:858, 859, and 860, respectively, and the TCR-a variable region comprises a CDR1, CDR2, and CDR3 of SEQ ID NOs:673, 674, and 866, respectively.

The polypeptide or engineered TCR may comprise a TCR-b variable region and a TCR-a variable region, wherein the TCR-b variable region comprises a CDR1, CDR2, and CDR3 of SEQ ID NOs:342, 343, and 870, respectively, and the TCR-a variable region comprises a CDR1, CDR2, and CDR3 of SEQ ID NOs:314, 315, and 874, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 877, respectively, and the TCR-a variable region comprises a CDR1, CDR2, and CDR3 of SEQ ID NOs:303, 304, and 880, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 883, respectively, and the TCR-a variable region comprises a CDR1, CDR2, and CDR3 of SEQ ID NOs:886, 887, and 888, respectively.

The polypeptide or engineered TCR may comprise a TCR-b variable region and a TCR-a variable region, wherein the TCR-b variable region comprises a CDR1, CDR2, and CDR3 of SEQ ID NOs:894, 895, and 896, respectively, and the TCR-a variable region comprises a CDR1, CDR2, and CDR3 of SEQ ID NOs:207, 208, and 902, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 906, respectively, and the TCR-a variable region comprises a CDR1, CDR2, and CDR3 of SEQ ID NOs:48, 49, and 909, respectively.

The polypeptide or engineered TCR may comprise a TCR-b variable region and a TCR-a variable region, wherein the TCR-b variable region comprises a CDR1, CDR2, and CDR3 of SEQ ID NOs:57, 58, and 913, respectively, and the TCR-a variable region comprises a CDR1, CDR2, and CDR3 of SEQ ID NOs:506, 507, and 916, respectively.

The polypeptide or engineered TCR may comprise a TCR-b variable region and a TCR-a variable region, wherein the TCR-b variable region comprises a CDR1, CDR2, and CDR3 of SEQ ID NOs:199, 200, and 919, respectively, and the TCR-a variable region comprises a CDR1, CDR2, and CDR3 of SEQ ID NOs:236, 237, and 922, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 925, respectively, and the TCR-a variable region comprises a CDR1, CDR2, and CDR3 of SEQ ID NOs:533, 534, and 928, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 931, respectively, and the TCR-a variable region comprises a CDR1, CDR2, and CDR3 of SEQ ID NOs:207, 208, and 937, respectively.

The polypeptide or engineered TCR may comprise a TCR-b variable region and a TCR-a variable region, wherein the TCR-b variable region comprises a CDR1, CDR2, and CDR3 of SEQ ID NOs:639, 640, and 934, respectively, and the TCR-a variable region comprises a CDR1, CDR2, and CDR3 of SEQ ID NOs:207, 208, and 937, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 940, respectively, and the TCR-a variable region comprises a CDR1, CDR2, and CDR3 of SEQ ID NOs:286, 287, and 943, respectively.

The polypeptide or engineered TCR may comprise a TCR-b variable region and a TCR-a variable region, wherein the TCR-b variable region comprises a CDR1, CDR2, and CDR3 of SEQ ID NOs:395, 396, and 946, respectively, and the TCR-a variable region comprises a CDR1, CDR2, and CDR3 of SEQ ID NOs:207, 208, and 949, respectively.

The polypeptide or engineered TCR may comprise a TCR-b variable region and a TCR-a variable region, wherein the TCR-b variable region comprises a CDR1, CDR2, and CDR3 of SEQ ID NOs:275, 276, and 952, respectively, and the TCR-a variable region comprises a CDR1, CDR2, and CDR3 of SEQ ID NOs:118, 119, and 955, respectively.

The polypeptide or engineered TCR may comprise a TCR-b variable region and a TCR-a variable region, wherein the TCR-b variable region comprises a CDR1, CDR2, and CDR3 of SEQ ID NOs:639, 640, and 958, respectively, and the TCR-a variable region comprises a CDR1, CDR2, and CDR3 of SEQ ID NOs:207, 208, and 961, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 964, respectively, and the TCR-a variable region comprises a CDR1, CDR2, and CDR3 of SEQ ID NOs:415, 416, and 967, respectively.

The polypeptide or engineered TCR may comprise a TCR-b variable region and a TCR-a variable region, wherein the TCR-b variable region comprises a CDR1, CDR2, and CDR3 of SEQ ID NOs:57, 58, and 970, respectively, and the TCR-a variable region comprises a CDR1, CDR2, and CDR3 of SEQ ID NOs:973, 974, and 975, respectively.

The polypeptide or engineered TCR may comprise a TCR-b variable region and a TCR-a variable region, wherein the TCR-b variable region comprises a CDR1, CDR2, and CDR3 of SEQ ID NOs:514, 515, and 981, respectively, and the TCR-a variable region comprises a CDR1, CDR2, and CDR3 of SEQ ID NOs:118, 119, and 984, respectively.

The polypeptide or engineered TCR may comprise a TCR-b variable region and a TCR-a variable region, wherein the TCR-b variable region comprises a CDR1, CDR2, and CDR3 of SEQ ID NOs:514, 515, and 987, respectively, and the TCR-a variable region comprises a CDR1, CDR2, and CDR3 of SEQ ID NOs:70, 71, and 990, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 993, respectively, and the TCR-a variable region comprises a CDR1, CDR2, and CDR3 of SEQ ID NOs:506, 507, and 996, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 999, respectively, and the TCR-a variable region comprises a CDR1, CDR2, and CDR3 of SEQ ID NOs:118, 119, and 1002, respectively.

The polypeptide or engineered TCR may comprise a TCR-b variable region and a TCR-a variable region, wherein the TCR-b variable region comprises a CDR1, CDR2, and CDR3 of SEQ ID NOs:1005, 276, and 1006, respectively, and the TCR-a variable region comprises a CDR1, CDR2, and CDR3 of SEQ ID NOs:506, 507, and 1012, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 1015, respectively, and the TCR-a variable region comprises a CDR1, CDR2, and CDR3 of SEQ ID NOs:12, 13, and 1018, respectively.

The polypeptide or engineered TCR may comprise a TCR-b variable region and a TCR-a variable region, wherein the TCR-b variable region comprises a CDR1, CDR2, and CDR3 of SEQ ID NOs:199, 200, and 681, respectively, and the TCR-a variable region comprises a CDR1, CDR2, and CDR3 of SEQ ID NOs:427, 428, and 684, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 1021, respectively, and the TCR-a variable region comprises a CDR1, CDR2, and CDR3 of SEQ ID NOs:153, 154, and 1024, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 1028, respectively, and the TCR-a variable region comprises a CDR1, CDR2, and CDR3 of SEQ ID NOs:831, 832, and 1031, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 1034, respectively, and the TCR-a variable region comprises a CDR1, CDR2, and CDR3 of SEQ ID NOs:303, 304, and 1037, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 1040, respectively, and the TCR-a variable region comprises a CDR1, CDR2, and CDR3 of SEQ ID NOs:403, 404, and 1043, respectively.

The polypeptide or engineered TCR may comprise a TCR-b variable region and a TCR-a variable region, wherein the TCR-b variable region comprises a CDR1, CDR2, and CDR3 of SEQ ID NOs:252, 1046, and 1047, respectively, and the TCR-a variable region comprises a CDR1, CDR2, and CDR3 of SEQ ID NOs:506, 507, and 1053, respectively.

The polypeptide or engineered TCR may comprise a TCR-b variable region and a TCR-a variable region, wherein the TCR-b variable region comprises a CDR1, CDR2, and CDR3 of SEQ ID NOs:169, 706, and 1056, respectively, and the TCR-a variable region comprises a CDR1, CDR2, and CDR3 of SEQ ID NOs:533, 534, and 1059, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1062, respectively, and the TCR-a variable region comprises a CDR1, CDR2, and CDR3 of SEQ ID NOs:106, 107, and 1066, respectively.

The polypeptide or engineered TCR may comprise a TCR-b variable region and a TCR-a variable region, wherein the TCR-b variable region comprises a CDR1, CDR2, and CDR3 of SEQ ID NOs:525, 526, and 1069, respectively, and the TCR-a variable region comprises a CDR1, CDR2, and CDR3 of SEQ ID NOs:1072, 1073, and 1074, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 1080, respectively, and the TCR-a variable region comprises a CDR1, CDR2, and CDR3 of SEQ ID NOs:673, 674, and 1084, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 1087, respectively, and the TCR-a variable region comprises a CDR1, CDR2, and CDR3 of SEQ ID NOs:12, 13, and 1090, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 1093, respectively, and the TCR-a variable region comprises a CDR1, CDR2, and CDR3 of SEQ ID NOs:552, 553, and 1096, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 1099, respectively, and the TCR-a variable region comprises a CDR1, CDR2, and CDR3 of SEQ ID NOs:70, 71, and 1102, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 1106, respectively, and the TCR-a variable region comprises a CDR1, CDR2, and CDR3 of SEQ ID NOs:48, 49, and 1109, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 1112, respectively, and the TCR-a variable region comprises a CDR1, CDR2, and CDR3 of SEQ ID NOs:1115, 287, and 1116, respectively.

The polypeptide or engineered TCR may comprise a TCR-b variable region and a TCR-a variable region, wherein the TCR-b variable region comprises a CDR1, CDR2, and CDR3 of SEQ ID NOs:468, 469, and 1119, respectively, and the TCR-a variable region comprises a CDR1, CDR2, and CDR3 of SEQ ID NOs:552, 553, and 1122, respectively.

The polypeptide or engineered TCR may comprise a TCR-b variable region and a TCR-a variable region, wherein the TCR-b variable region comprises a CDR1, CDR2, and CDR3 of SEQ ID NOs:1125, 1126, and 1127, respectively, and the TCR-a variable region comprises a CDR1, CDR2, and CDR3 of SEQ ID NOs:886, 887, and 1133, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 1137, respectively, and the TCR-a variable region comprises a CDR1, CDR2, and CDR3 of SEQ ID NOs:48, 49, and 1140, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 1143, respectively, and the TCR-a variable region comprises a CDR1, CDR2, and CDR3 of SEQ ID NOs:118, 119, and 1146, respectively.

The polypeptide or engineered TCR may comprise a TCR-b variable region and a TCR-a variable region, wherein the TCR-b variable region comprises a CDR1, CDR2, and CDR3 of SEQ ID NOs:514, 515, and 1149, respectively, and the TCR-a variable region comprises a CDR1, CDR2, and CDR3 of SEQ ID NOs:106, 107, and 1152, respectively.

The polypeptide or engineered TCR may comprise a TCR-b variable region and a TCR-a variable region, wherein the TCR-b variable region comprises a CDR1, CDR2, and CDR3 of SEQ ID NOs:514, 515, and 1155, respectively, and the TCR-a variable region comprises a CDR1, CDR2, and CDR3 of SEQ ID NOs:118, 119, and 1158, respectively.

The polypeptide or engineered TCR may comprise a TCR-b variable region and a TCR-a variable region, wherein the TCR-b variable region comprises a CDR1, CDR2, and CDR3 of SEQ ID NOs:331, 332, and 1161, respectively, and the TCR-a variable region comprises a CDR1, CDR2, and CDR3 of SEQ ID NOs:552, 553, and 1164, respectively.

The polypeptide or engineered TCR may comprise a TCR-b variable region and a TCR-a variable region, wherein the TCR-b variable region comprises a CDR1, CDR2, and CDR3 of SEQ ID NOs:894, 1167, and 1168, respectively, and the TCR-a variable region comprises a CDR1, CDR2, and CDR3 of SEQ ID NOs:106, 107, and 1174, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 1177, respectively, and the TCR-a variable region comprises a CDR1, CDR2, and CDR3 of SEQ ID NOs:450, 451, and 1180, respectively.

The polypeptide or engineered TCR may comprise a TCR-b variable region and a TCR-a variable region, wherein the TCR-b variable region comprises a CDR1, CDR2, and CDR3 of SEQ ID NOs:252, 1046, and 1184, respectively, and the TCR-a variable region comprises a CDR1, CDR2, and CDR3 of SEQ ID NOs:314, 315, and 1187, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1190, respectively, and the TCR-a variable region comprises a CDR1, CDR2, and CDR3 of SEQ ID NOs:762, 763, and 1193, respectively.

The polypeptide or engineered TCR may comprise a TCR-b variable region and a TCR-a variable region, wherein the TCR-b variable region comprises a CDR1, CDR2, and CDR3 of SEQ ID NOs:468, 469, and 1196, respectively, and the TCR-a variable region comprises a CDR1, CDR2, and CDR3 of SEQ ID NOs:48, 49, and 1199, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 1202, respectively, and the TCR-a variable region comprises a CDR1, CDR2, and CDR3 of SEQ ID NOs:207, 208, and 1205, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 1208, respectively, and the TCR-a variable region comprises a CDR1, CDR2, and CDR3 of SEQ ID NOs:118, 119, and 1211, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 1214, respectively, and the TCR-a variable region comprises a CDR1, CDR2, and CDR3 of SEQ ID NOs:973, 974, and 1217, respectively.

The polypeptide or engineered TCR may comprise a TCR-b variable region and a TCR-a variable region, wherein the TCR-b variable region comprises a CDR1, CDR2, and CDR3 of SEQ ID NOs:39, 40, and 1220, respectively, and the TCR-a variable region comprises a CDR1, CDR2, and CDR3 of SEQ ID NOs:450, 451, and 1223, respectively.

The polypeptide or engineered TCR may comprise a TCR-b variable region and a TCR-a variable region, wherein the TCR-b variable region comprises a CDR1, CDR2, and CDR3 of SEQ ID NOs:3, 4, and 1226, respectively, and the TCR-a variable region comprises a CDR1, CDR2, and CDR3 of SEQ ID NOs:533, 534, and 1229, respectively.

The polypeptide or engineered TCR may comprise a TCR-b variable region and a TCR-a variable region, wherein the TCR-b variable region comprises a CDR1, CDR2, and CDR3 of SEQ ID NOs:514, 515, and 1232, respectively, and the TCR-a variable region comprises a CDR1, CDR2, and CDR3 of SEQ ID NOs:506, 507, and 1235, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 1238, respectively, and the TCR-a variable region comprises a CDR1, CDR2, and CDR3 of SEQ ID NOs:1072, 1073, and 1241, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 1244, respectively, and the TCR-a variable region comprises a CDR1, CDR2, and CDR3 of SEQ ID NOs:495, 496, and 1247, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 1251, respectively, and the TCR-a variable region comprises a CDR1, CDR2, and CDR3 of SEQ ID NOs:207, 208, and 1254, respectively.

The polypeptide or engineered TCR may comprise a TCR-b variable region and a TCR-a variable region, wherein the TCR-b variable region comprises a CDR1, CDR2, and CDR3 of SEQ ID NOs:894, 895, and 1257, respectively, and the TCR-a variable region comprises a CDR1, CDR2, and CDR3 of SEQ ID NOs:224, 225, and 1260, respectively.

The polypeptide or engineered TCR may comprise a TCR-b variable region and a TCR-a variable region, wherein the TCR-b variable region comprises a CDR1, CDR2, and CDR3 of SEQ ID NOs:252, 1046, and 1264, respectively, and the TCR-a variable region comprises a CDR1, CDR2, and CDR3 of SEQ ID NOs:427, 428, and 1267, respectively.

The polypeptide or engineered TCR may comprise a TCR-b variable region and a TCR-a variable region, wherein the TCR-b variable region comprises a CDR1, CDR2, and CDR3 of SEQ ID NOs:639, 640, and 1270, respectively, and the TCR-a variable region comprises a CDR1, CDR2, and CDR3 of SEQ ID NOs:314, 315, and 1273, respectively.

The polypeptide or engineered TCR may comprise a TCR-b variable region and a TCR-a variable region, wherein the TCR-b variable region comprises a CDR1, CDR2, and CDR3 of SEQ ID NOs:468, 469, and 1276, respectively, and the TCR-a variable region comprises a CDR1, CDR2, and CDR3 of SEQ ID NOs:224, 225, and 1279, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1282, respectively, and the TCR-a variable region comprises a CDR1, CDR2, and CDR3 of SEQ ID NOs:673, 674, and 1285, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1288, respectively, and the TCR-a variable region comprises a CDR1, CDR2, and CDR3 of SEQ ID NOs:506, 507, and 1291, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1294, respectively, and the TCR-a variable region comprises a CDR1, CDR2, and CDR3 of SEQ ID NOs:450, 451, and 1297, respectively.

The polypeptide or engineered TCR may comprise a TCR-b variable region and a TCR-a variable region, wherein the TCR-b variable region comprises a CDR1, CDR2, and CDR3 of SEQ ID NOs:21, 22, and 1300, respectively, and the TCR-a variable region comprises a CDR1, CDR2, and CDR3 of SEQ ID NOs:12, 13, and 1303, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 1307, respectively, and the TCR-a variable region comprises a CDR1, CDR2, and CDR3 of SEQ ID NOs:673, 674, and 1310, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1313, respectively, and the TCR-a variable region comprises a CDR1, CDR2, and CDR3 of SEQ ID NOs:12, 13, and 1316, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1319, respectively, and the TCR-a variable region comprises a CDR1, CDR2, and CDR3 of SEQ ID NOs:286, 287, and 1322, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 1325, respectively, and the TCR-a variable region comprises a CDR1, CDR2, and CDR3 of SEQ ID NOs:70, 71, and 1328, respectively.

The polypeptide or engineered TCR may comprise a TCR-b variable region and a TCR-a variable region, wherein the TCR-b variable region comprises a CDR1, CDR2, and CDR3 of SEQ ID NOs:169, 170, and 1331, respectively, and the TCR-a variable region comprises a CDR1, CDR2, and CDR3 of SEQ ID NOs:236, 237, and 1334, respectively.

The polypeptide or engineered TCR may comprise a TCR-b variable region and a TCR-a variable region, wherein the TCR-b variable region comprises a CDR1, CDR2, and CDR3 of SEQ ID NOs:894, 895, and 1337, respectively, and the TCR-a variable region comprises a CDR1, CDR2, and CDR3 of SEQ ID NOs:575, 576, and 1340, respectively.

The polypeptide or engineered TCR may comprise a TCR-b variable region and a TCR-a variable region, wherein the TCR-b variable region comprises a CDR1, CDR2, and CDR3 of SEQ ID NOs:514, 515, and 1343, respectively, and the TCR-a variable region comprises a CDR1, CDR2, and CDR3 of SEQ ID NOs:118, 119, and 1346, respectively.

The polypeptide or engineered TCR may comprise a TCR-b variable region and a TCR-a variable region, wherein the TCR-b variable region comprises a CDR1, CDR2, and CDR3 of SEQ ID NOs:514, 515, and 1343, respectively, and the TCR-a variable region comprises a CDR1, CDR2, and CDR3 of SEQ ID NOs:552, 553, and 1349, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 503, respectively, and the TCR-a variable region comprises a CDR1, CDR2, and CDR3 of SEQ ID NOs:506, 507, and 508, respectively.

The polypeptide or engineered TCR may comprise a TCR-b variable region and a TCR-a variable region, wherein the TCR-b variable region comprises a CDR1, CDR2, and CDR3 of SEQ ID NOs:514, 515, and 516, respectively, and the TCR-a variable region comprises a CDR1, CDR2, and CDR3 of SEQ ID NOs:153, 154, and 522, respectively.

The polypeptide or engineered TCR may comprise a TCR-b variable region and a TCR-a variable region, wherein the TCR-b variable region comprises a CDR1, CDR2, and CDR3 of SEQ ID NOs:525, 526, and 527, respectively, and the TCR-a variable region comprises a CDR1, CDR2, and CDR3 of SEQ ID NOs:533, 534, and 535, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 542, respectively, and the TCR-a variable region comprises a CDR1, CDR2, and CDR3 of SEQ ID NOs:286, 287, and 545, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 549, respectively, and the TCR-a variable region comprises a CDR1, CDR2, and CDR3 of SEQ ID NOs:552, 553, and 554, respectively.

The polypeptide or engineered TCR may comprise a TCR-b variable region and a TCR-a variable region, wherein the TCR-b variable region comprises a CDR1, CDR2, and CDR3 of SEQ ID NOs:127, 128, and 560, respectively, and the TCR-a variable region comprises a CDR1, CDR2, and CDR3 of SEQ ID NOs:135, 136, and 563, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 566, respectively, and the TCR-a variable region comprises a CDR1, CDR2, and CDR3 of SEQ ID NOs:135, 136, and 569, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 572, respectively, and the TCR-a variable region comprises a CDR1, CDR2, and CDR3 of SEQ ID NOs:575, 576, and 577, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 583, respectively, and the TCR-a variable region comprises a CDR1, CDR2, and CDR3 of SEQ ID NOs:118, 119, and 586, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 590, respectively, and the TCR-a variable region comprises a CDR1, CDR2, and CDR3 of SEQ ID NOs:594, 595, and 596, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 603, respectively, and the TCR-a variable region comprises a CDR1, CDR2, and CDR3 of SEQ ID NOs:575, 576, and 606, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 610, respectively, and the TCR-a variable region comprises a CDR1, CDR2, and CDR3 of SEQ ID NOs:303, 304, and 613, respectively.

The polypeptide or engineered TCR may comprise a TCR-b variable region and a TCR-a variable region, wherein the TCR-b variable region comprises a CDR1, CDR2, and CDR3 of SEQ ID NOs:295, 296, and 616, respectively, and the TCR-a variable region comprises a CDR1, CDR2, and CDR3 of SEQ ID NOs:450, 451, and 619, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 622, respectively, and the TCR-a variable region comprises a CDR1, CDR2, and CDR3 of SEQ ID NOs:303, 304, and 625, respectively.

The polypeptide or engineered TCR may comprise a TCR-b variable region and a TCR-a variable region, wherein the TCR-b variable region comprises a CDR1, CDR2, and CDR3 of SEQ ID NOs:263, 629, and 630, respectively, and the TCR-a variable region comprises a CDR1, CDR2, and CDR3 of SEQ ID NOs:594, 595, and 636, respectively.

The polypeptide or engineered TCR may comprise a TCR-b variable region and a TCR-a variable region, wherein the TCR-b variable region comprises a CDR1, CDR2, and CDR3 of SEQ ID NOs:639, 640, and 641, respectively, and the TCR-a variable region comprises a CDR1, CDR2, and CDR3 of SEQ ID NOs:450, 451, and 647, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 650, respectively, and the TCR-a variable region comprises a CDR1, CDR2, and CDR3 of SEQ ID NOs:314, 315, and 653, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 656, respectively, and the TCR-a variable region comprises a CDR1, CDR2, and CDR3 of SEQ ID NOs:659, 660, and 661, respectively.

The polypeptide or engineered TCR may comprise a TCR-b variable region and a TCR-a variable region, wherein the TCR-b variable region comprises a CDR1, CDR2, and CDR3 of SEQ ID NOs:665, 666, and 667, respectively, and the TCR-a variable region comprises a CDR1, CDR2, and CDR3 of SEQ ID NOs:673, 674, and 675, respectively.

The polypeptide or engineered TCR may comprise a TCR-b variable region and a TCR-a variable region, wherein the TCR-b variable region comprises a CDR1, CDR2, and CDR3 of SEQ ID NOs:199, 200, and 681, respectively, and the TCR-a variable region comprises a CDR1, CDR2, and CDR3 of SEQ ID NOs:427, 428, and 684, respectively.

The polypeptide or engineered TCR may comprise a TCR-b variable region and a TCR-a variable region, wherein the TCR-b variable region comprises a CDR1, CDR2, and CDR3 of SEQ ID NOs:468, 469, and 688, respectively, and the TCR-a variable region comprises a CDR1, CDR2, and CDR3 of SEQ ID NOs:118, 119, and 691, respectively.

The polypeptide or engineered TCR may comprise a TCR-b variable region and a TCR-a variable region, wherein the TCR-b variable region comprises a CDR1, CDR2, and CDR3 of SEQ ID NOs:665, 666, and 694, respectively, and the TCR-a variable region comprises a CDR1, CDR2, and CDR3 of SEQ ID NOs:427, 428, and 697, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 700, respectively, and the TCR-a variable region comprises a CDR1, CDR2, and CDR3 of SEQ ID NOs:594, 595, and 703, respectively.

The polypeptide or engineered TCR may comprise a TCR-b variable region and a TCR-a variable region, wherein the TCR-b variable region comprises a CDR1, CDR2, and CDR3 of SEQ ID NOs:169, 706, and 707, respectively, and the TCR-a variable region comprises a CDR1, CDR2, and CDR3 of SEQ ID NOs:506, 507, and 711, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 714, respectively, and the TCR-a variable region comprises a CDR1, CDR2, and CDR3 of SEQ ID NOs:207, 208, and 717, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 777, respectively, and the TCR-a variable region comprises a CDR1, CDR2, and CDR3 of SEQ ID NOs:207, 208, and 780, respectively.

The polypeptide or engineered TCR may comprise a TCR-b variable region and a TCR-a variable region, wherein the TCR-b variable region comprises a CDR1, CDR2, and CDR3 of SEQ ID NOs:665, 666, and 783, respectively, and the TCR-a variable region comprises a CDR1, CDR2, and CDR3 of SEQ ID NOs:118, 119, and 786, respectively.

The polypeptide or engineered TCR may comprise a TCR-b variable region and a TCR-a variable region, wherein the TCR-b variable region comprises a CDR1, CDR2, and CDR3 of SEQ ID NOs:275, 443, and 790, respectively, and the TCR-a variable region comprises a CDR1, CDR2, and CDR3 of SEQ ID NOs:314, 315, and 793, respectively.

The polypeptide or engineered TCR may comprise a TCR-b variable region and a TCR-a variable region, wherein the TCR-b variable region comprises a CDR1, CDR2, and CDR3 of SEQ ID NOs:199, 200, and 799, respectively, and the TCR-a variable region comprises a CDR1, CDR2, and CDR3 of SEQ ID NOs:450, 451, and 802, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 805, respectively, and the TCR-a variable region comprises a CDR1, CDR2, and CDR3 of SEQ ID NOs:12, 13, and 808, respectively.

The polypeptide or engineered TCR may comprise a TCR-b variable region and a TCR-a variable region, wherein the TCR-b variable region comprises a CDR1, CDR2, and CDR3 of SEQ ID NOs:97, 98, and 805, respectively, and the TCR-a variable region comprises a CDR1, CDR2, and CDR3 of SEQ ID NOs:286, 287, and 811, respectively.

The polypeptide or engineered TCR may comprise a TCR-b variable region and a TCR-a variable region, wherein the TCR-b variable region comprises a CDR1, CDR2, and CDR3 of SEQ ID NOs:144, 145, and 815, respectively, and the TCR-a variable region comprises a CDR1, CDR2, and CDR3 of SEQ ID NOs:88, 89, and 819, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 822, respectively, and the TCR-a variable region comprises a CDR1, CDR2, and CDR3 of SEQ ID NOs:403, 404, and 825, respectively.

The polypeptide or engineered TCR may comprise a TCR-b variable region and a TCR-a variable region, wherein the TCR-b variable region comprises a CDR1, CDR2, and CDR3 of SEQ ID NOs:21, 22, and 828, respectively, and the TCR-a variable region comprises a CDR1, CDR2, and CDR3 of SEQ ID NOs:831, 832, and 833, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 906, respectively, and the TCR-a variable region comprises a CDR1, CDR2, and CDR3 of SEQ ID NOs:48, 49, and 909, respectively.

The polypeptide or engineered TCR may comprise a TCR-b variable region and a TCR-a variable region, wherein the TCR-b variable region comprises a CDR1, CDR2, and CDR3 of SEQ ID NOs:57, 58, and 913, respectively, and the TCR-a variable region comprises a CDR1, CDR2, and CDR3 of SEQ ID NOs:506, 507, and 916, respectively.

The polypeptide or engineered TCR may comprise a TCR-b variable region and a TCR-a variable region, wherein the TCR-b variable region comprises a CDR1, CDR2, and CDR3 of SEQ ID NOs:199, 200, and 919, respectively, and the TCR-a variable region comprises a CDR1, CDR2, and CDR3 of SEQ ID NOs:236, 237, and 922, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 925, respectively, and the TCR-a variable region comprises a CDR1, CDR2, and CDR3 of SEQ ID NOs:533, 534, and 928, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 931, respectively, and the TCR-a variable region comprises a CDR1, CDR2, and CDR3 of SEQ ID NOs:207, 208, and 937, respectively.

The polypeptide or engineered TCR may comprise a TCR-b variable region and a TCR-a variable region, wherein the TCR-b variable region comprises a CDR1, CDR2, and CDR3 of SEQ ID NOs:639, 640, and 934, respectively, and the TCR-a variable region comprises a CDR1, CDR2, and CDR3 of SEQ ID NOs:207, 208, and 937, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 940, respectively, and the TCR-a variable region comprises a CDR1, CDR2, and CDR3 of SEQ ID NOs:286, 287, and 943, respectively.

The polypeptide or engineered TCR may comprise a TCR-b variable region and a TCR-a variable region, wherein the TCR-b variable region comprises a CDR1, CDR2, and CDR3 of SEQ ID NOs:199, 200, and 681, respectively, and the TCR-a variable region comprises a CDR1, CDR2, and CDR3 of SEQ ID NOs:427, 428, and 684, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 1021, respectively, and the TCR-a variable region comprises a CDR1, CDR2, and CDR3 of SEQ ID NOs:153, 154, and 1024, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 1028, respectively, and the TCR-a variable region comprises a CDR1, CDR2, and CDR3 of SEQ ID NOs:831, 832, and 1031, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 1034, respectively, and the TCR-a variable region comprises a CDR1, CDR2, and CDR3 of SEQ ID NOs:303, 304, and 1037, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 1040, respectively, and the TCR-a variable region comprises a CDR1, CDR2, and CDR3 of SEQ ID NOs:403, 404, and 1043, respectively.

The polypeptide or engineered TCR may comprise a TCR-b variable region and a TCR-a variable region, wherein the TCR-b variable region comprises a CDR1, CDR2, and CDR3 of SEQ ID NOs:252, 1046, and 1047, respectively, and the TCR-a variable region comprises a CDR1, CDR2, and CDR3 of SEQ ID NOs:506, 507, and 1053, respectively.

The polypeptide or engineered TCR may comprise a TCR-b variable region and a TCR-a variable region, wherein the TCR-b variable region comprises a CDR1, CDR2, and CDR3 of SEQ ID NOs:169, 706, and 1056, respectively, and the TCR-a variable region comprises a CDR1, CDR2, and CDR3 of SEQ ID NOs:533, 534, and 1059, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1062, respectively, and the TCR-a variable region comprises a CDR1, CDR2, and CDR3 of SEQ ID NOs:106, 107, and 1066, respectively.

The polypeptide or engineered TCR may comprise a TCR-b variable region and a TCR-a variable region, wherein the TCR-b variable region comprises a CDR1, CDR2, and CDR3 of SEQ ID NOs:525, 526, and 1069, respectively, and the TCR-a variable region comprises a CDR1, CDR2, and CDR3 of SEQ ID NOs:1072, 1073, and 1074, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 1080, respectively, and the TCR-a variable region comprises a CDR1, CDR2, and CDR3 of SEQ ID NOs:673, 674, and 1084, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 1087, respectively, and the TCR-a variable region comprises a CDR1, CDR2, and CDR3 of SEQ ID NOs:12, 13, and 1090, respectively.

The polypeptide or engineered TCR may comprise a TCR-b variable region and a TCR-a variable region, wherein the TCR-b variable region comprises a CDR1, CDR2, and CDR3 of SEQ ID NOs:252, 253, and 1093, respectively, and the TCR-a variable region comprises a CDR1, CDR2, and CDR3 of SEQ ID NOs:552, 553, and 1096, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 1099, respectively, and the TCR-a variable region comprises a CDR1, CDR2, and CDR3 of SEQ ID NOs:70, 71, and 1102, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 1106, respectively, and the TCR-a variable region comprises a CDR1, CDR2, and CDR3 of SEQ ID NOs:48, 49, and 1109, respectively.

The polypeptide or engineered TCR may comprise a TCR-b variable region and a TCR-a variable region, wherein the TCR-b variable region comprises a CDR1, CDR2, and CDR3 of SEQ ID NOs:442, 443, and 1112, respectively, and the TCR-a variable region comprises a CDR1, CDR2, and CDR3 of SEQ ID NOs:1115, 287, and 1116, respectively.

The polypeptide or engineered TCR may comprise a TCR-b variable region and a TCR-a variable region, wherein the TCR-b variable region comprises a CDR1, CDR2, and CDR3 of SEQ ID NOs:468, 469, and 1119, respectively, and the TCR-a variable region comprises a CDR1, CDR2, and CDR3 of SEQ ID NOs:552, 553, and 1122, respectively.

The polypeptide or engineered TCR may comprise a TCR-b variable region and a TCR-a variable region, wherein the TCR-b variable region comprises a CDR1, CDR2, and CDR3 of SEQ ID NOs:1125, 1126, and 1127, respectively, and the TCR-a variable region comprises a CDR1, CDR2, and CDR3 of SEQ ID NOs:886, 887, and 1133, respectively.

The polypeptide or engineered TCR may comprise a TCR-b variable region and a TCR-a variable region, wherein the TCR-b variable region comprises a CDR1, CDR2, and CDR3 of SEQ ID NOs:383, 384, and 1137, respectively, and the TCR-a variable region comprises a CDR1, CDR2, and CDR3 of SEQ ID NOs:48, 49, and 1140, respectively.

The polypeptide or engineered TCR may comprise a TCR-b variable region and a TCR-a variable region, wherein the TCR-b variable region comprises a CDR1, CDR2, and CDR3 of SEQ ID NOs:252, 366, and 1143, respectively, and the TCR-a variable region comprises a CDR1, CDR2, and CDR3 of SEQ ID NOs:118, 119, and 1146, respectively.

The polypeptide or engineered TCR may comprise a TCR-b variable region and a TCR-a variable region, wherein the TCR-b variable region comprises a CDR1, CDR2, and CDR3 of SEQ ID NOs:252, 1046, and 1264, respectively, and the TCR-a variable region comprises a CDR1, CDR2, and CDR3 of SEQ ID NOs:427, 428, and 1267, respectively.

The polypeptide or engineered TCR may comprise a TCR-b variable region and a TCR-a variable region, wherein the TCR-b variable region comprises a CDR1, CDR2, and CDR3 of SEQ ID NOs:639, 640, and 1270, respectively, and the TCR-a variable region comprises a CDR1, CDR2, and CDR3 of SEQ ID NOs:314, 315, and 1273, respectively.

The polypeptide or engineered TCR may comprise a TCR-b variable region and a TCR-a variable region, wherein the TCR-b variable region comprises a CDR1, CDR2, and CDR3 of SEQ ID NOs:468, 469, and 1276, respectively, and the TCR-a variable region comprises a CDR1, CDR2, and CDR3 of SEQ ID NOs:224, 225, and 1279, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1282, respectively, and the TCR-a variable region comprises a CDR1, CDR2, and CDR3 of SEQ ID NOs:673, 674, and 1285, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1288, respectively, and the TCR-a variable region comprises a CDR1, CDR2, and CDR3 of SEQ ID NOs:506, 507, and 1291, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1294, respectively, and the TCR-a variable region comprises a CDR1, CDR2, and CDR3 of SEQ ID NOs:450, 451, and 1297, respectively.

The polypeptide or engineered TCR may comprise a TCR-b variable region and a TCR-a variable region, wherein the TCR-b variable region comprises a CDR1, CDR2, and CDR3 of SEQ ID NOs:21, 22, and 1300, respectively, and the TCR-a variable region comprises a CDR1, CDR2, and CDR3 of SEQ ID NOs:12, 13, and 1303, respectively.

The polypeptide or engineered TCR may comprise a TCR-b variable region and a TCR-a variable region, wherein the TCR-b variable region comprises a CDR1, CDR2, and CDR3 of SEQ ID NOs:216, 217, and 1307, respectively, and the TCR-a variable region comprises a CDR1, CDR2, and CDR3 of SEQ ID NOs:673, 674, and 1310, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1313, respectively, and the TCR-a variable region comprises a CDR1, CDR2, and CDR3 of SEQ ID NOs:12, 13, and 1316, respectively.

The polypeptide or engineered TCR may comprise a TCR-b variable region and a TCR-a variable region, wherein the TCR-b variable region comprises a CDR1, CDR2, and CDR3 of SEQ ID NOs:79, 80, and 1319, respectively, and the TCR-a variable region comprises a CDR1, CDR2, and CDR3 of SEQ ID NOs:286, 287, and 1322, respectively. The polypeptide or engineered TCR may comprise a TCR-b variable region and a TCR-a variable region, wherein the TCR-b variable region and TCR-a variable region comprises SEQ ID NOs: 2 and 11; SEQ ID NOs: 20 and 29; SEQ ID NOs: 38 and 47; SEQ ID NOs: 56 and 69; SEQ ID NOs: 65 and 69; SEQ ID NOs: 78 and 87; SEQ ID NOs: 96 and 105; SEQ ID NOs: 114 and 117; SEQ ID NOs: 126 and 134; SEQ ID NOs: 143 and 152; SEQ ID NOs: 160 and 176; SEQ ID NOs: 168 and 176; SEQ ID NOs: 180 and 188; SEQ ID NOs: 192 and 195; SEQ ID NOs: 198 and 206; SEQ ID NOs: 215 and 223; SEQ ID NOs: 232 and 235; SEQ ID NOs: 244 and 248; SEQ ID NOs: 251 and 258; SEQ ID NOs: 262 and 270; SEQ ID NOs: 274 and 285; SEQ ID NOs: 282 and 285; SEQ ID NOs: 294 and 302; SEQ ID NOs: 310 and 313; SEQ ID NOs: 322 and 325; SEQ ID NOs: 328 and 337; SEQ ID NOs: 330 and 337; SEQ ID NOs: 341 and 349; SEQ ID NOs: 358 and 361; SEQ ID NOs: 365 and 372; SEQ ID NOs: 376 and 379; SEQ ID NOs: 382 and 390; SEQ ID NOs: 394 and 402; SEQ ID NOs: 411 and 414; SEQ ID NOs: 422 and 426; SEQ ID NOs: 434 and 437; SEQ ID NOs: 441 and 449; SEQ ID NOs: 458 and 464; SEQ ID NOs: 461 and 464; SEQ ID NOs: 467 and 248; SEQ ID NOs: 475 and 478; SEQ ID NOs: 482 and 485; SEQ ID NOs: 491 and 494; SEQ ID NOs: 502 and 505; SEQ ID NOs: 513 and 521; SEQ ID NOs: 524 and 532; SEQ ID NOs: 541 and 544; SEQ ID NOs: 548 and 551; SEQ ID NOs: 559 and 562; SEQ ID NOs: 565 and 568; SEQ ID NOs: 571 and 574; SEQ ID NOs: 582 and 585; SEQ ID NOs: 589 and 593; SEQ ID NOs: 602 and 605; SEQ ID NOs: 609 and 612; SEQ ID NOs: 615 and 618; SEQ ID NOs: 621 and 624; SEQ ID NOs: 628 and 635; SEQ ID NOs: 638 and 646; SEQ ID NOs: 649 and 652; SEQ ID NOs: 655 and 658; SEQ ID NOs: 664 and 672; SEQ ID NOs: 680 and 683; SEQ ID NOs: 687 and 690; SEQ ID NOs: 693 and 696; SEQ ID NOs: 699 and 702; SEQ ID NOs: 705 and 710; SEQ ID NOs: 713 and 716; SEQ ID NOs: 719 and 722; SEQ ID NOs: 726 and 729; SEQ ID NOs: 732 and 735; SEQ ID NOs: 738 and 741; SEQ ID NOs: 744 and 749; SEQ ID NOs: 752 and 755; SEQ ID NOs: 758 and 761; SEQ ID NOs: 769 and 772; SEQ ID NOs: 776 and 779; SEQ ID NOs: 782 and 785; SEQ ID NOs: 789 and 795; SEQ ID NOs: 798 and 801; SEQ ID NOs: 804 and 807; SEQ ID NOs: 804 and 810; SEQ ID NOs: 814 and 818; SEQ ID NOs: 821 and 824; SEQ ID NOs: 827 and 830; SEQ ID NOs: 838 and 841; SEQ ID NOs: 844 and 852; SEQ ID NOs: 857 and 865; SEQ ID NOs: 869 and 873; SEQ ID NOs: 876 and 879; SEQ ID NOs: 882 and 885; SEQ ID NOs: 893 and 901; SEQ ID NOs: 905 and 908; SEQ ID NOs: 912 and 915; SEQ ID NOs: 918 and 921; SEQ ID NOs: 924 and 927; SEQ ID NOs: 930 and 936; SEQ ID NOs: 933 and 936; SEQ ID NOs: 939 and 942; SEQ ID NOs: 945 and 948; SEQ ID NOs: 951 and 954; SEQ ID NOs: 957 and 960; SEQ ID NOs: 963 and 966; SEQ ID NOs: 969 and 972; SEQ ID NOs: 980 and 983; SEQ ID NOs: 986 and 989; SEQ ID NOs: 992 and 995; SEQ ID NOs: 998 and 1001; SEQ ID NOs: 1004 and 1011; SEQ ID NOs: 1014 and 1017; SEQ ID NOs: 680 and 683; SEQ ID NOs: 1020 and 1023; SEQ ID NOs: 1027 and 1030; SEQ ID NOs: 1033 and 1036; SEQ ID NOs: 1039 and 1042; SEQ ID NOs: 1045 and 1052; SEQ ID NOs: 1055 and 1058; SEQ ID NOs: 1061 and 1065; SEQ ID NOs: 1068 and 1071; SEQ ID NOs: 1079 and 1083; SEQ ID NOs: 1086 and 1089; SEQ ID NOs: 1092 and 1095; SEQ ID NOs: 1098 and 1101; SEQ ID NOs: 1105 and 1108; SEQ ID NOs: 1111 and 1114; SEQ ID NOs: 1118 and 1121; SEQ ID NOs: 1124 and 1132; SEQ ID NOs: 1136 and 1139; SEQ ID NOs: 1142 and 1145; SEQ ID NOs: 1148 and 1151; SEQ ID NOs: 1154 and 1157; SEQ ID NOs: 1160 and 1163; SEQ ID NOs: 1166 and 1173; SEQ ID NOs: 1176 and 1179; SEQ ID NOs: 1183 and 1186; SEQ ID NOs: 1189 and 1192; SEQ ID NOs: 1195 and 1198; SEQ ID NOs: 1201 and 1204; SEQ ID NOs: 1207 and 1210; SEQ ID NOs: 1213 and 1216; SEQ ID NOs: 1219 and 1222; SEQ ID NOs: 1225 and 1228; SEQ ID NOs: 1231 and 1234; SEQ ID NOs: 1237 and 1240; SEQ ID NOs: 1243 and 1246; SEQ ID NOs: 1250 and 1253; SEQ ID NOs: 1256 and 1259; SEQ ID NOs: 1263 and 1266; SEQ ID NOs: 1269 and 1272; SEQ ID NOs: 1275 and 1278; SEQ ID NOs: 1281 and 1284; SEQ ID NOs: 1287 and 1290; SEQ ID NOs: 1293 and 1296; SEQ ID NOs: 1299 and 1302; SEQ ID NOs: 1306 and 1309; SEQ ID NOs: 1312 and 1315; SEQ ID NOs: 1318 and 1321; SEQ ID NOs: 1324 and 1327; SEQ ID NOs: 1330 and 1333; SEQ ID NOs: 1336 and 1339; SEQ ID NOs: 1342 and 1345; SEQ ID NOs: 1342 and 1348; SEQ ID NOs: 502 and 505; SEQ ID NOs: 513 and 521; SEQ ID NOs: 524 and 532; SEQ ID NOs: 541 and 544; SEQ ID NOs: 548 and 551; SEQ ID NOs: 559 and 562; SEQ ID NOs: 565 and 568; SEQ ID NOs: 571 and 574; SEQ ID NOs: 582 and 585; SEQ ID NOs: 589 and 593; SEQ ID NOs: 602 and 605; SEQ ID NOs: 609 and 612; SEQ ID NOs: 615 and 618; SEQ ID NOs: 621 and 624; SEQ ID NOs: 628 and 635; SEQ ID NOs: 638 and 646; SEQ ID NOs: 649 and 652; SEQ ID NOs: 655 and 658; SEQ ID NOs: 664 and 672; SEQ ID NOs: 680 and 683; SEQ ID NOs: 687 and 690; SEQ ID NOs: 693 and 696; SEQ ID NOs: 699 and 702; SEQ ID NOs: 705 and 710; SEQ ID NOs: 713 and 716; SEQ ID NOs: 776 and 779; SEQ ID NOs: 782 and 785; SEQ ID NOs: 789 and 795; SEQ ID NOs: 798 and 801; SEQ ID NOs: 804 and 807; SEQ ID NOs: 804 and 810; SEQ ID NOs: 814 and 818; SEQ ID NOs: 821 and 824; SEQ ID NOs: 827 and 830; SEQ ID NOs: 905 and 908; SEQ ID NOs: 912 and 915; SEQ ID NOs: 918 and 921; SEQ ID NOs: 924 and 927; SEQ ID NOs: 930 and 936; SEQ ID NOs: 933 and 936; SEQ ID NOs: 939 and 942; SEQ ID NOs: 680 and 683; SEQ ID NOs: 1020 and 1023; SEQ ID NOs: 1027 and 1030; SEQ ID NOs: 1033 and 1036; SEQ ID NOs: 1039 and 1042; SEQ ID NOs: 1045 and 1052; SEQ ID NOs: 1055 and 1058; SEQ ID NOs: 1061 and 1065; SEQ ID NOs: 1068 and 1071; SEQ ID NOs: 1079 and 1083; SEQ ID NOs: 1086 and 1089; SEQ ID NOs: 1092 and 1095; SEQ ID NOs: 1098 and 1101; SEQ ID NOs: 1105 and 1108; SEQ ID NOs: 1111 and 1114; SEQ ID NOs: 1118 and 1121; SEQ ID NOs: 1124 and 1132; SEQ ID NOs: 1136 and 1139; SEQ ID NOs: 1142 and 1145; SEQ ID NOs: 1263 and 1266; SEQ ID NOs: 1269 and 1272; SEQ ID NOs: 1275 and 1278; SEQ ID NOs: 1281 and 1284; SEQ ID NOs: 1287 and 1290; SEQ ID NOs: 1293 and 1296; SEQ ID NOs: 1299 and 1302; SEQ ID NOs: 1306 and 1309; SEQ ID NOs: 1312 and 1315; or SEQ ID NOs: 1318 and 1321.

“Treatment” or treating may refer to any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some aspects, the treatment may exclude prevention of the disease.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### DETAILED DESCRIPTION OF THE INVENTION

### I. Engineered T Cell Receptors

T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T-cell receptor differ from the membrane-bound immunoglobulin that serves as the B-cell receptor: a T-cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Cα domain, where the fold is unlike that of any other immunoglobulin-like domain. The half of the domain that is juxtaposed with the Cβ domain forms a β sheet similar to that found in other immunoglobulin-like domains, but the other half of the domain is formed of loosely packed strands and a short segment of α helix. The intramolecular disulfide bond, which in immunoglobulin-like domains normally joins two β strands, in a Cα domain joins a β strand to this segment of a helix.

There are also differences in the way in which the domains interact. The interface between the V and C domains of both T-cell receptor chains is more extensive than in antibodies, which may make the hinge joint between the domains less flexible. And the interaction between the Cα and Cβ domains is distinctive in being assisted by carbohydrate, with a sugar group from the Cα domain making a number of hydrogen bonds to the Cβ domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the β strand that anchors one end of the loop from one face of the domain to the other. A strand displacement also causes a change in the orientation of the Vβ CDR2 loop in two of the seven Vβ domains whose structures are known. As yet, the crystallographic structures of seven T-cell receptors have been solved to this level of resolution.

Aspects of the disclosure relate to engineered T cell receptors. The term “engineered” refers to T cell receptors that have TCR variable regions grafted onto TCR constant regions to make a chimeric polypeptide that binds to peptides and antigens of the disclosure. In certain aspects, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some aspects, the TCR comprises non-TCR sequences. Accordingly, certain aspects relate to TCRs with sequences that are not from a TCR gene. In some aspects, the TCR is chimeric, in that it contains sequences normally found in a TCR gene, but contains sequences from at least two TCR genes that are not necessarily found together in nature.

In some aspects the engineered TCRs of the disclosure comprise a variable as shown below. TCRB refers to a beta chain, and TCRA refers to an alpha chain. b-variable nucleotide refers to the nucleotide sequence of the beta chain variable region, and variable-b refers to the amino acid sequence of the beta chain variable region. CDR1-b, CDR2-b, and CDR3-b refer to the CDRs of the beta chain, and FWR1-b, FWR2-b, FWR3-b, and FWR4-b refer to the framework regions of the beta chain variable region. a-variable nucleotide refers to the nucleotide sequence of the alpha chain variable region, and variable-a refers to the amino acid sequence of the alpha chain variable region. CDR1-a, CDR2-a, and CDR3-a refer to the CDRs of the alpha chain, and FWR1-a, FWR2-a, FWR3-a, and FWR4-a refer to the framework regions of the alpha chain variable region. In some instances, the clone # has a X.1 or X.2, where X is the clone number. The “0.1” or “0.2” refers to alternative chains that can be used with the corresponding alpha or beta chain. For example, the table below lists a TCRB-4.1, TCR1B4.2, and TCRA-4. The TCR may comprise a TCRB-4.1 and TCRA-4 or a TCRB-4.2 and TCRA-4. Furthermore, aspects that discuss the “corresponding clone of Table 1” refer to the clone # in the Table below. In the example above, the clone # is 4. In the Table below, the clone # follows the TCR gene in the format of TCRB-X, TCRA-X, TCRB-X.1, TCRB-X.2, TCRA-X.1, or TCRA-X.2, wherein X is the clone number.

TABLE 1
TCR Clones

| TCR | | | SEQ |
|---|---|---|---|
| gene- | | | ID |
| clone# | Description | Sequence | NO: |
| TCRB- | b-Variable | GATGCCATGGTCATCCAGAACCCAAGATACCAGGTTACCCAGTTT | 1 |
| 1 | nucleotide | GGAAAGCCAGTGACCCTGAGTTGTTCTCAGACTTTGAACCATAAC | |
| | | GTCATGTACTGGTACCAGCAGAAGTCAAGTCAGGCCCCAAAGCT | |
| | | GCTGTTCCACTACTATGACAAAGATTTTAACAATGAAGCAGACAC | |
| | | CCCTGATAACTTCCAATCCAGGAGGCCGAACACTTCTTTCTGCTTT | |
| | | CTTGACATCCGCTCACCAGGCCTGGGGGACGCAGCCATGTACCTG | |
| | | TGTGCCACCAGCAGAGGCCCGGGGGCCTCGGAAGATACGCAGTA | |
| | | TTTTGGCCCAGGCACCCGGCTGACAGTGCTCG | |
| | Variable-b | DAMVIQNPRYQVTQFGKPVTLSCSQTLNHNVMYWYQQKSSQAPKL | 2 |
| | | LFHYYDKDENNEADTPDNFQSRRPNTSFCFLDIRSPGLGDAAMYLCA | |
| | | TSRGPGASEDTQYFGPGTRLTVL | |
| | CDR1-b | LNHNV | 3 |
| | CDR2-b | YYDKDF | 4 |
| | CDR3-b | CATSRGPGASEDTQYF | 5 |
| | FWR1-b | DAMVIQNPRYQVTQFGKPVTLSCSQT | 6 |
| | FWR2-b | MYWYQQKSSQAPKLLFH | 7 |
| | FWR3-b | NNEADTPDNFQSRRPNTSFCFLDIRSPGLGDAAMYL | 8 |
| | FWR4-b | GPGTRLTVL | 9 |
| TCRA- | a-Variable | CAGAAGGAGGTGGAGCAGAATTCTGGACCCCTCAGTGTTCCAGA | 10 |
| 1 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTTCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAATGAGTATGGAAACAAACTGGTCTTTGGCGCAGGAACC | |
| | | ATTCTGAGAGTCAAGTCCT | |
| | Variable-a | QKEVEQNSGPLSVPEGAIASLNCTYSDRGSQSFFWYRQYSGKSPELI | 11 |
| | | MFIYSNGDKEDGRFTAQLNKASQYVSLLIRDSQPSDSATYLCAVNEY | |
| | | GNKLVFGAGTILRVKS | |
| | CDR1-a | DRGSQS | 12 |
| | CDR2-a | IYSNGD | 13 |
| | CDR3-a | CAVNEYGNKLVF | 14 |
| | FWR1-a | QKEVEQNSGPLSVPEGAIASLNCTYS | 15 |
| | FWR2-a | FFWYRQYSGKSPELIMF | 16 |
| | FWR3-a | KEDGRFTAQLNKASQYVSLLIRDSQPSDSATYL | 17 |
| | FWR4-a | GAGTILRVKS | 18 |
| TCRB- | b-Variable | GATGCTGATGTTACCCAGACCCCAAGGAATAGGATCACAAAGAC | 19 |
| 2 | nucleotide | AGGAAAGAGGATTATGCTGGAATGTTCTCAGACTAAGGGTCATG | |
| | | ATAGAATGTACTGGTATCGACAAGACCCAGGACTGGGCCTACGG | |
| | | TTGATCTATTACTCCTTTGATGTCAAAGATATAAACAAAGGAGAG | |
| | | ATCTCTGATGGATACAGTGTCTCTCGACAGGCACAGGCTAAATTC | |
| | | TCCCTGTCCCTAGAGTCTGCCATCCCCAACCAGACAGCTCTTTACT | |
| | | TCTGTGCCACCAGTGATCTAAGTATCAATCAGCCCCAGCATTTTG | |
| | | GTGATGGGACTCGACTCTCCATCCTAG | |
| | Variable-b | DADVTQTPRNRITKTGKRIMLECSQTKGHDRMYWYRQDPGLGLRLI | 20 |
| | | YYSFDVKDINKGEISDGYSVSRQAQAKFSLSLESAIPNQTALYFCATS | |
| | | DLSINQPQHFGDGTRLSIL | |
| | CDR1-b | KGHDR | 21 |
| | CDR2-b | SFDVKD | 22 |
| | CDR3-b | CATSDLSINQPQHF | 23 |
| | FWR1-b | DADVTQTPRNRITKTGKRIMLECSQT | 24 |
| | FWR2-b | MYWYRQDPGLGLRLIYY | 25 |
| | FWR3-b | INKGEISDGYSVSRQAQAKFSLSLESAIPNQTALYF | 26 |
| | FWR4-b | GDGTRLSIL | 27 |
| TCRA- | a-Variable | CAACAACCAGTGCAGAGTCCTCAAGCCGTGATCCTCCGAGAAGG | 28 |
| 2 | nucleotide | GGAAGATGCTGTCATCAACTGCAGTTCCTCCAAGGCTTTATATTC | |
| | | TGTACACTGGTACAGGCAGAAGCATGGTGAAGCACCCATCTTCCT | |
| | | GATGATATTACTGAAGGGTGGAGAACAGAAGGGTCATGACAAAA | |
| | | TATCTGCTTCATTTAATGAAAAAAAGCAGCAAAGCTCCCTGTACC | |
| | | TTACGGCCTCCCAGCTCAGTTACTCAGGAACCTACTTCTGTGGCA | |
| | | CAGAGATAGGGACCGACAAGCTCATCTTTGGGACTGGGACCAGA | |
| | | TTACAAGTCTTTCCAA | |
| | Variable-a | QQPVQSPQAVILREGEDA VINCSSSKALYSVHWYRQKHGEAPIFLMI | 29 |
| | | LLKGGEQKGHDKISASFNEKKQQSSLYLTASQLSYSGTYFCGTEIGT | |
| | | DKLIFGTGTRLQVFP | |
| | CDR1-a | KALYS | 30 |
| | CDR2-a | LLKGGEQ | 31 |
| | CDR3-a | CGTEIGTDKLIF | 32 |
| | FWR1-a | QQPVQSPQA VILREGEDA VINCSSS | 33 |
| | FWR2-a | VHWYRQKHGEAPIFLMI | 34 |
| | FWR3-a | KGHDKISASFNEKKQQSSLYLTASQLSYSGTYF | 35 |
| | FWR4-a | GTGTRLQVFP | 36 |
| TCRB- | b-Variable | AATGCTGGTGTCACTCAGACCCCAAAATTCCAGGTCCTGAAGACA | 37 |
| 3 | nucleotide | GGACAGAGCATGACACTGCAGTGTGCCCAGGATATGAACCATAA | |
| | | CTCCATGTACTGGTATCGACAAGACCCAGGCATGGGACTGAGGCT | |
| | | GATTTATTACTCAGCTTCTGAGGGTACCACTGACAAAGGAGAAGT | |
| | | CCCCAATGGCTACAATGTCTCCAGATTAAACAAACGGGAGTTCTC | |
| | | GCTCAGGCTGGAGTCGGCTGCTCCCTCCCAGACATCTGTGTACTT | |
| | | CTGTGCCAGCAGTTACCTCATCGGGGCGGCCGATGAAAAACTGTT | |
| | | TTTTGGCAGTGGAACCCAGCTCTCTGTCTTGG | |
| | Variable-b | NAGVTQTPKFQVLKTGQSMTLQCAQDMNHNSMYWYRQDPGMGL | 38 |
| | | RLIYYSASEGTTDKGEVPNGYNVSRLNKREFSLRLESAAPSQTSVYF | |
| | | CASSYLIGAADEKLFFGSGTQLSVL | |
| | CDR1-b | MNHNS | 39 |
| | CDR2-b | SASEGT | 40 |
| | CDR3-b | CASSYLIGAADEKLFF | 41 |
| | FWR1-b | NAGVTQTPKFQVLKTGQSMTLQCAQD | 42 |
| | FWR2-b | MYWYRQDPGMGLRLIYY | 43 |
| | FWR3-b | TDKGEVPNGYNVSRLNKREFSLRLESAAPSQTSVYF | 44 |
| | FWR4-b | GSGTQLSVL | 45 |
| TCRA- | a-Variable | GCTCAGAAGGTAACTCAAGCGCAGACTGAAATTTCTGTGGTGGA | 46 |
| 3 | nucleotide | GAAGGAGGATGTGACCTTGGACTGTGTGTATGAAACCCGTGATA | |
| | | CTACTTATTACTTATTCTGGTACAAGCAACCACCAAGTGGAGAAT | |
| | | TGGTTTTCCTTATTCGTCGGAACTCTTTTGATGAGCAAAATGAAAT | |
| | | AAGTGGTCGGTATTCTTGGAACTTCCAGAAATCCACCAGTTCCTT | |
| | | CAACTTCACCATCACAGCCTCACAAGTCGTGGACTCAGCAGTATA | |
| | | CTTCTGTGCTCTGAGTGAGAAGGAGACAACTGACAGCTGGGGGA | |
| | | AATTGCAGTTTGGAGCAGGGACCCAGGTTGTGGTCACCCCAG | |
| | Variable-a | AQKVTQAQTEISVVEKEDVTLDCVYETRDTTYYLFWYKQPPSGELV | 47 |
| | | FLIRRNSFDEQNEISGRYSWNFQKSTSSFNFTITASQVVDSAVYFCAL | |
| | | SEKETTDSWGKLQFGAGTQVVVTP | |
| | CDR1-a | TRDTTYY | 48 |
| | CDR2-a | RNSFDEQN | 49 |
| | CDR3-a | CALSEKETTDSWGKLQF | 50 |
| | FWR1-a | AQKVTQAQTEISVVEKEDVTLDCVYE | 51 |
| | FWR2-a | LFWYKQPPSGEL VFLIR | 52 |
| | FWR3-a | EISGRYSWNFQKSTSSFNFTITASQVVDSAVYF | 53 |
| | FWR4-a | GAGTQVVVTP | 54 |
| TCRB- | b-Variable | GAAGCTGGAGTTACTCAGTTCCCCAGCCACAGCGTAATAGAGAA | 55 |
| 4.1 | nucleotide | GGGCCAGACTGTGACTCTGAGATGTGACCCAATTTCTGGACATGA | |
| | | TAATCTTTATTGGTATCGACGTGTTATGGGAAAAGAAATAAAATT | |
| | | TCTGTTACATTTTGTGAAAGAGTCTAAACAGGATGAGTCCGGTAT | |
| | | GCCCAACAATCGATTCTTAGCTGAAAGGACTGGAGGGACGTATTC | |
| | | TACTCTGAAGGTGCAGCCTGCAGAACTGGAGGATTCTGGAGTTTA | |
| | | TTTCTGTGCCAGCAGCCAAGATGGGGGCCGCAGGGGGCTGTGGA | |
| | | CCGGGGAGCTGTTTTTTGGAGAAGGCTCTAGGCTGACCGTACTGG | |
| | Variable-b | EAGVTQFPSHSVIEKGQTVTLRCDPISGHDNLYWYRRVMGKEIKFLL | 56 |
| | | HFVKESKQDESGMPNNRFLAERTGGTYSTLKVQPAELEDSGVYFCA | |
| | | SSQDGGRRGLWTGELFFGEGSRLTVL | |
| | CDR1-b | SGHDN | 57 |
| | CDR2-b | FVKESK | 58 |
| | CDR3-b | CASSQDGGRRGLWTGELFF | 59 |
| | FWR1-b | EAGVTQFPSHSVIEKGQTVTLRCDPI | 60 |
| | FWR2-b | LYWYRRVMGKEIKFLLH | 61 |
| | FWR3-b | QDESGMPNNRFLAERTGGTYSTLKVQPAELEDSGVYF | 62 |
| | FWR4-b | GEGSRLTVL | 63 |
| TCRB- | b-Variable | GATGCTGATGTTACCCAGACCCCAAGGAATAGGATCACAAAGAC | 64 |
| 4.2 | nucleotide | AGGAAAGAGGATTATGCTGGAATGTTCTCAGACTAAGGGTCATG | |
| | | ATAGAATGTACTGGTATCGACAAGACCCAGGACTGGGCCTACGG | |
| | | TTGATCTATTACTCCTTTGATGTCAAAGATATAAACAAAGGAGAG | |
| | | ATCTCTGATGGATACAGTGTCTCTCGACAGGCACAGGCTAAATTC | |
| | | TCCCTGTCCCTAGAGTCTGCCATCCCCAACCAGACAGCTCTTTACT | |
| | | TCTGTGCCACCAGTGATCCCCACGGGACTAGCGGGGTCAGACAA | |
| | | GAGACCCAGTACTTCGGGCCAGGCACGCGGCTCCTGGTGCTCG | |
| | Variable-b | DADVTQTPRNRITKTGKRIMLECSQTKGHDRMYWYRQDPGLGLRLI | 65 |
| | | YYSFDVKDINKGEISDGYSVSRQAQAKFSLSLESAIPNQTALYFCATS | |
| | | DPHGTSGVRQETQYFGPGTRLLVL | |
| | CDR1-b | KGHDR | 21 |
| | CDR2-b | SFDVKD | 22 |
| | CDR3-b | CATSDPHGTSGVRQETQYF | 66 |
| | FWR1-b | DADVTQTPRNRITKTGKRIMLECSQT | 24 |
| | FWR2-b | MYWYRQDPGLGLRLIYY | 25 |
| | FWR3-b | INKGEISDGYSVSRQAQAKFSLSLESAIPNQTALYF | 26 |
| | FWR4-b | GPGTRLLVL | 67 |
| TCRA- | a-Variable | GCCCAGAGAGTGACTCAGCCCGAGAAGCTCCTCTCTGTCTTTAAA | 68 |
| 4 | nucleotide | GGGGCCCCAGTGGAGCTGAAGTGCAACTATTCCTATTCTGGGAGT | |
| | | CCTGAACTCTTCTGGTATGTCCAGTACTCCAGACAACGCCTCCAG | |
| | | TTACTCTTGAGACACATCTCTAGAGAGAGCATCAAAGGCTTCACT | |
| | | GCTGACCTTAACAAAGGCGAGACATCTTTCCACCTGAAGAAACC | |
| | | ATTTGCTCAAGAGGAAGACTCAGCCATGTATTACTGTGCTGCCAC | |
| | | TAATGCTGGTGGTACTAGCTATGGAAAGCTGACATTTGGACAAGG | |
| | | GACCATCTTGACTGTCCATCCAA | |
| | Variable-a | AQRVTQPEKLLSVFKGAPVELKCNYSYSGSPELFWYVQYSRQRLQL | 69 |
| | | LLRHISRESIKGFTADLNKGETSFHLKKPFAQEEDSAMYYCAATNAG | |
| | | GTSYGKLTFGQGTILTVHP | |
| | CDR1-a | YSGSPE | 70 |
| | CDR2-a | HISR | 71 |
| | CDR3-a | CAATNAGGTSYGKLTF | 72 |
| | FWR1-a | AQRVTQPEKLLSVFKGAPVELKCNYS | 73 |
| | FWR2-a | LFWYVQYSRQRLQLLLR | 74 |
| | FWR3-a | ESIKGFTADLNKGETSFHLKKPFAQEEDSAMYY | 75 |
| | FWR4-a | GQGTILTVHP | 76 |
| TCRB- | b-Variable | GGTGCTGTCGTCTCTCAACATCCGAGCTGGGTTATCTGTAAGAGT | 77 |
| 5 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTGATAGCGGGGGAACCCCCTACGAGC | |
| | | AGTACTTCGGGCCGGGCACCAGGCTCACGGTCACAG | |
| | Variable-b | GAVVSQHPSWVICKSGTSVKIECRSLDFQATTMFWYRQFPKQSLML | 78 |
| | | MATSNEGSKATYEQGVEKDKFLINHASLTLSTLTVTSAHPEDSSFYIC | |
| | | SADSGGTPYEQYFGPGTRLTVT | |
| | CDR1-b | DFQATT | 79 |
| | CDR2-b | SNEGSKA | 80 |
| | CDR3-b | CSADSGGTPYEQYF | 81 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVT | 85 |
| TCRA- | a-Variable | GATGCTAAGACCACCCAGCCCACCTCCATGGATTGCGCTGAAGG | 86 |
| 5 | nucleotide | AAGAGCTGCAAACCTGCCTTGTAATCACTCTACCATCAGTGGAAA | |
| | | TGAGTATGTGTATTGGTATCGACAGATTCACTCCCAGGGGCCACA | |
| | | GTATATCATTCATGGTCTAAAAAACAATGAAACCAATGAAATGG | |
| | | CCTCTCTGATCATCACAGAAGACAGAAAGTCCAGCACCTTGATCC | |
| | | TGCCCCACGCTACGCTGAGAGACACTGCTGTGTACTATTGCATCG | |
| | | TCAGAGCGAACACCGGTAACCAGTTCTATTTTGGGACAGGGACA | |
| | | AGTTTGACGGTCATTCCAA | |
| | Variable-a | DAKTTQPTSMDCAEGRAANLPCNHSTISGNEYVYWYRQIHSQGPQY | 87 |
| | | IIHGLKNNETNEMASLIITEDRKSSTLILPHATLRDTAVYYCIVRANTG | |
| | | NQFYFGTGTSLTVIP | |
| | CDR1-a | TISGNEY | 88 |
| | CDR2-a | GLKNN | 89 |
| | CDR3-a | CIVRANTGNQFYF | 90 |
| | FWR1-a | DAKTTQPTSMDCAEGRAANLPCNHS | 91 |
| | FWR2-a | VYWYRQIHSQGPQYIIH | 92 |
| | FWR3-a | ETNEMASLIITEDRKSSTLILPHATLRDTAVYY | 93 |
| | FWR4-a | GTGTSLTVIP | 94 |
| TCRB- | b-Variable | AGTGCTGTCATCTCTCAAAAGCCAAGCAGGGATATCTGTCAACGT | 95 |
| 6 | nucleotide | GGAACCTCCCTGACGATCCAGTGTCAAGTCGATAGCCAAGTCACC | |
| | | ATGATGTTCTGGTACCGTCAGCAACCTGGACAGAGCCTGACACTG | |
| | | ATCGCAACTGCAAATCAGGGCTCTGAGGCCACATATGAGAGTGG | |
| | | ATTTGTCATTGACAAGTTTCCCATCAGCCGCCCAAACCTAACATT | |
| | | CTCAACTCTGACTGTGAGCAACATGAGCCCTGAAGACAGCAGCA | |
| | | TATATCTCTGCAGCGTTGAAGAGGGGGACAGATACTATGGCTACA | |
| | | CCTTCGGTTCGGGGACCAGGTTAACCGTTGTAG | |
| | Variable-b | SAVISQKPSRDICQRGTSLTIQCQVDSQVTMMFWYRQQPGQSLTLIA | 96 |
| | | TANQGSEATYESGFVIDKFPISRPNLTFSTLTVSNMSPEDSSIYLCSVE | |
| | | EGDRYYGYTFGSGTRLTVV | |
| | CDR1-b | SQVTM | 97 |
| | CDR2-b | ANQGSEA | 98 |
| | CDR3-b | CSVEEGDRYYGYTF | 99 |
| | FWR1-b | SAVISQKPSRDICQRGTSLTIQCQVD | 100 |
| | FWR2-b | MFWYRQQPGQSLTLIAT | 101 |
| | FWR3-b | TYESGFVIDKFPISRPNLTFSTLTVSNMSPEDSSIYL | 102 |
| | FWR4-b | GSGTRLTVV | 103 |
| TCRA- | a-Variable | GGAGATTCAGTGACCCAGATGGAAGGGCCAGTGACTCTCTCAGA | 104 |
| 6 | nucleotide | AGAGGCCTTCCTGACTATAAACTGCACGTACACAGCCACAGGAT | |
| | | ACCCTTCCCTTTTCTGGTATGTCCAATATCCTGGAGAAGGTCTACA | |
| | | GCTCCTCCTGAAAGCCACGAAGGCTGATGACAAGGGAAGCAACA | |
| | | AAGGTTTTGAAGCCACATACCGTAAAGAAACCACTTCTTTCCACT | |
| | | TGGAGAAAGGCTCAGTTCAAGTGTCAGACTCAGCGGTGTACTTCT | |
| | | GTGCTCTGAGCTGGTCGGATGGTGGAGGCTTCAAAACTATCTTTG | |
| | | GAGCAGGAACAAGACTATTTGTTAAAGCAA | |
| | Variable-a | GDSVTQMEGPVTLSEEAFLTINCTYTATGYPSLFWYVQYPGEGLQLL | 105 |
| | | LKATKADDKGSNKGFEATYRKETTSFHLEKGSVQVSDSAVYFCALS | |
| | | WSDGGGFKTIFGAGTRLFVKA | |
| | CDR1-a | ATGYPS | 106 |
| | CDR2-a | ATKADDK | 107 |
| | CDR3-a | CALSWSDGGGFKTIF | 108 |
| | FWR1-a | GDSVTQMEGPVTLSEEAFLTINCTYT | 109 |
| | FWR2-a | LFWYVQYPGEGLQLLLK | 110 |
| | FWR3-a | GSNKGFEATYRKETTSFHLEKGSVQVSDSAVYF | 111 |
| | FWR4-a | GAGTRLFVKA | 112 |
| TCRB- | b-Variable | GGTGCTGTCGTCTCTCAACATCCGAGCTGGGTTATCTGTAAGAGT | 113 |
| 7 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCCAGGGGGGTGGGGTCCCCTTCCTACG | |
| | | AGCAGTACTTCGGGCCGGGCACCAGGCTCACGGTCACAG | |
| | Variable-b | GAVVSQHPSWVICKSGTSVKIECRSLDFQATTMFWYRQFPKQSLML | 114 |
| | | MATSNEGSKATYEQGVEKDKFLINHASLTLSTLTVTSAHPEDSSFYIC | |
| | | SARGVGSPSYEQYFGPGTRLTVT | |
| | CDR1-b | DFQATT | 79 |
| | CDR2-b | SNEGSKA | 80 |
| | CDR3-b | CSARGVGSPSYEQYF | 115 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVT | 85 |
| TCRA- | a-Variable | AAACAGGAGGTGACGCAGATTCCTGCAGCTCTGAGTGTCCCAGA | 116 |
| 7 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGCAGGGAAGCCAAGGAAATCTCATCTTTGGAAAAGGCA | |
| | | CTAAACTCTCTGTTAAACCAA | |
| | Variable-a | KQEVTQIPAALSVPEGENLVLNCSFTDSAIYNLQWFRQDPGKGLTSL | 117 |
| | | LLIQSSQREQTSGRLNASLDKSSGRSTLYIAASQPGDSATYLCAVQGS | |
| | | QGNLIFGKGTKLSVKP | |
| | CDR1-a | DSAIYN | 118 |
| | CDR2-a | IQSSQRE | 119 |
| | CDR3-a | CAVQGSQGNLIF | 120 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GKGTKLSVKP | 124 |
| TCRB- | b-Variable | GAGACTGGAGTCACCCAAAGTCCCACACACCTGATCAAAACGAG | 125 |
| 8 | nucleotide | AGGACAGCAAGTGACTCTGAGATGCTCTTCTCAGTCTGGGCACAA | |
| | | CACTGTGTCCTGGTACCAACAGGCCCTGGGTCAGGGGCCCCAGTT | |
| | | TATCTTTCAGTATTATAGGGAGGAAGAGAATGGCAGAGGAAACT | |
| | | TCCCTCCTAGATTCTCAGGTCTCCAGTTCCCTAATTATAGCTCTGA | |
| | | GCTGAATGTGAACGCCTTGGAGCTGGACGACTCGGCCCTGTATCT | |
| | | CTGTGCCAGCAGCTCGGATAAGGCCCCTTCCCAGTACTTCGGGCC | |
| | | AGGCACGCGGCTCCTGGTGCTCG | |
| | Variable-b | ETGVTQSPTHLIKTRGQQVTLRCSSQSGHNTVSWYQQALGQGPQFIF | 126 |
| | | QYYREEENGRGNFPPRFSGLQFPNYSSELNVNALELDDSALYLCASS | |
| | | SDKAPSQYFGPGTRLLVL | |
| | CDR1-b | SGHNT | 127 |
| | CDR2-b | YYREEE | 128 |
| | CDR3-b | CASSSDKAPSQYF | 129 |
| | FWR1-b | ETGVTQSPTHLIKTRGQQVTLRCSSQ | 130 |
| | FWR2-b | VSWYQQALGQGPQFIFQ | 131 |
| | FWR3-b | NGRGNFPPRFSGLQFPNYSSELNVNALELDDSALYL | 132 |
| | FWR4-b | GPGTRLLVL | 67 |
| TCRA- | a-Variable | AAAAACCAAGTGGAGCAGAGTCCTCAGTCCCTGATCATCCTGGA | 133 |
| 8 | nucleotide | GGGAAAGAACTGCACTCTTCAATGCAATTATACAGTGAGCCCCTT | |
| | | CAGCAACTTAAGGTGGTATAAGCAAGATACTGGGAGAGGTCCTG | |
| | | TTTCCCTGACAATCATGACTTTCAGTGAGAACACAAAGTCGAACG | |
| | | GAAGATATACAGCAACTCTGGATGCAGACACAAAGCAAAGCTCT | |
| | | CTGCACATCACAGCCTCCCAGCTCAGCGATTCAGCCTCCTACATC | |
| | | TGTGTGGTGAACAATGCAGGCAACATGCTCACCTTTGGAGGGGG | |
| | | AACAAGGTTAATGGTCAAACCCC | |
| | Variable-a | KNQVEQSPQSLIILEGKNCTLQCNYTVSPFSNLRWYKQDTGRGPVSL | 134 |
| | | TIMTFSENTKSNGRYTATLDADTKQSSLHITASQLSDSASYICVVNNA | |
| | | GNMLTFGGGTRLMVKP | |
| | CDR1-a | VSPFSN | 135 |
| | CDR2-a | MTFSENT | 136 |
| | CDR3-a | CVVNNAGNMLTF | 137 |
| | FWR1-a | KNQVEQSPQSLIILEGKNCTLQCNYT | 138 |
| | FWR2-a | LRWYKQDTGRGPVSLTI | 139 |
| | FWR3-a | KSNGRYTATLDADTKQSSLHITASQLSDSASYI | 140 |
| | FWR4-a | GGGTRLMVKP | 141 |
| TCRB- | b-Variable | AATGCCGGCGTCATGCAGAACCCAAGACACCTGGTCAGGAGGAG | 142 |
| 9 | nucleotide | GGGACAGGAGGCAAGACTGAGATGCAGCCCAATGAAAGGACAC | |
| | | AGTCATGTTTACTGGTATCGGCAGCTCCCAGAGGAAGGTCTGAAA | |
| | | TTCATGGTTTATCTCCAGAAAGAAAATATCATAGATGAGTCAGGA | |
| | | ATGCCAAAGGAACGATTTTCTGCTGAATTTCCCAAAGAGGGCCCC | |
| | | AGCATCCTGAGGATCCAGCAGGTAGTGCGAGGAGATTCGGCAGC | |
| | | TTATTTCTGTGCCAGCTCACTAGGGACGGGAAATTCACCCCTCCA | |
| | | CTTTGGGAACGGGACCAGGCTCACTGTGACAG | |
| | Variable-b | NAGVMQNPRHLVRRRGQEARLRCSPMKGHSHVYWYRQLPEEGLKF | 143 |
| | | MVYLQKENIIDESGMPKERFSAEFPKEGPSILRIQQVVRGDSAAYFCA | |
| | | SSLGTGNSPLHFGNGTRLTVT | |
| | CDR1-b | KGHSH | 144 |
| | CDR2-b | LQKENI | 145 |
| | CDR3-b | CASSLGTGNSPLHF | 146 |
| | FWR1-b | NAGVMQNPRHLVRRRGQEARLRCSPM | 147 |
| | FWR2-b | VYWYRQLPEEGLKFMVY | 148 |
| | FWR3-b | IDESGMPKERFSAEFPKEGPSILRIQQVVRGDSAAYF | 149 |
| | FWR4-b | GNGTRLTVT | 150 |
| TCRA- | a-Variable | GATGCTAAGACCACACAGCCAAATTCAATGGAGAGTAACGAAGA | 151 |
| 9 | nucleotide | AGAGCCTGTTCACTTGCCTTGTAACCACTCCACAATCAGTGGAAC | |
| | | TGATTACATACATTGGTATCGACAGCTTCCCTCCCAGGGTCCAGA | |
| | | GTACGTGATTCATGGTCTTACAAGCAATGTGAACAACAGAATGGC | |
| | | CTCTCTGGCAATCGCTGAAGACAGAAAGTCCAGTACCTTGATCCT | |
| | | GCACCGTGCTACCTTGAGAGATGCTGCTGTGTACTACTGCATCCT | |
| | | GAGAGACTATGATAATGCAGGCAACATGCTCACCTTTGGAGGGG | |
| | | GAACAAGGTTAATGGTCAAACCCC | |
| | Variable-a | DAKTTQPNSMESNEEEPVHLPCNHSTISGTDYIHWYRQLPSQGPEYV | 152 |
| | | IHGLTSNVNNRMASLAIAEDRKSSTLILHRATLRDAAVYYCILRDYD | |
| | | NAGNMLTFGGGTRLMVKP | |
| | CDR1-a | TISGTDY | 153 |
| | CDR2-a | GLTSN | 154 |
| | CDR3-a | CILRDYDNAGNMLTF | 155 |
| | FWR1-a | DAKTTQPNSMESNEEEPVHLPCNHS | 156 |
| | FWR2-a | IHWYRQLPSQGPEYVIH | 157 |
| | FWR3-a | VNNRMASLAIAEDRKSSTLILHRATLRDAAVYY | 158 |
| | FWR4-a | GGGTRLMVKP | 141 |
| TCRB- | b-Variable | GATGCTGGAATCACCCAGAGCCCAAGATACAAGATCACAGAGAC | 159 |
| 10.1 | nucleotide | AGGAAGGCAGGTGACCTTGATGTGTCACCAGACTTGGAGCCACA | |
| | | GCTATATGTTCTGGTATCGACAAGACCTGGGACATGGGCTGAGGC | |
| | | TGATCTATTACTCAGCAGCTGCTGATATTACAGATAAAGGAGAAG | |
| | | TCCCCGATGGCTACGTTGTCTCCAGATCCAAGACAGAGAATTTCC | |
| | | CCCTCACTCTGGAGTCAGCTACCCGCTCCCAGACATCTGTGTATTT | |
| | | CTGCGCCAGCAGCCCCGGCGAAGGGGTTGGGGGCTACACCTTCG | |
| | | GTTCGGGGACCAGGTTAACCGTTGTAG | |
| | Variable-b | DAGITQSPRYKITETGRQVTLMCHQTWSHSYMFWYRQDLGHGLRLI | 160 |
| | | YYSAAADITDKGEVPDGYVVSRSKTENFPLTLESATRSQTSVYFCAS | |
| | | SPGEGVGGYTFGSGTRLTVV | |
| | CDR1-b | WSHSY | 161 |
| | CDR2-b | SAAADI | 162 |
| | CDR3-b | CASSPGEGVGGYTF | 163 |
| | FWR1-b | DAGITQSPRYKITETGRQVTLMCHQT | 164 |
| | FWR2-b | MFWYRQDLGHGLRLIYY | 165 |
| | FWR3-b | TDKGEVPDGYVVSRSKTENFPLTLESATRSQTSVYF | 166 |
| | FWR4-b | GSGTRLTVV | 103 |
| TCRB- | b-Variable | GAAACGGGAGTTACGCAGACACCAAGACACCTGGTCATGGGAAT | 167 |
| 10.2 | nucleotide | GACAAATAAGAAGTCTTTGAAATGTGAACAACATCTGGGGCATA | |
| | | ACGCTATGTATTGGTACAAGCAAAGTGCTAAGAAGCCACTGGAG | |
| | | CTCATGTTTGTCTACAACTTTAAAGAACAGACTGAAAACAACAGT | |
| | | GTGCCAAGTCGCTTCTCACCTGAATGCCCCAACAGCTCTCACTTA | |
| | | TTCCTTCACCTACACACCCTGCAGCCAGAAGACTCGGCCCTGTAT | |
| | | CTCTGTGCCAGCAGCCAAACCACGACAGGGGGAGGGGACTACAC | |
| | | CTTCGGTTCGGGGACCAGGTTAACCGTTGTAG | |
| | Variable-b | ETGVTQTPRHLVMGMTNKKSLKCEQHLGHNAMYWYKQSAKKPLE | 168 |
| | | LMFVYNFKEQTENNSVPSRFSPECPNSSHLFLHLHTLQPEDSALYLC | |
| | | ASSQTTTGGGDYTFGSGTRLTVV | |
| | CDR1-b | LGHNA | 169 |
| | CDR2-b | YNFKEQ | 170 |
| | CDR3-b | CASSQTTTGGGDYTF | 171 |
| | FWR1-b | ETGVTQTPRHLVMGMTNKKSLKCEQH | 172 |
| | FWR2-b | MYWYKQSAKKPLELMFV | 173 |
| | FWR3-b | TENNSVPSRFSPECPNSSHLFLHLHTLQPEDSALYL | 174 |
| | FWR4-b | GSGTRLTVV | 103 |
| TCRA- | a-Variable | CAGAAGGAGGTGGAGCAGAATTCTGGACCCCTCAGTGTTCCAGA | 175 |
| 10 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTTCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAACGCCGGGGGTTACCAGAAAGTTACCTTTGGAATTGGA | |
| | | ACAAAGCTCCAAGTCATCCCAA | |
| | Variable-a | QKEVEQNSGPLSVPEGAIASLNCTYSDRGSQSFFWYRQYSGKSPELI | 176 |
| | | MFIYSNGDKEDGRFTAQLNKASQYVSLLIRDSQPSDSATYLCAVNA | |
| | | GGYQKVTFGIGTKLQVIP | |
| | CDR1-a | DRGSQS | 12 |
| | CDR2-a | IYSNGD | 13 |
| | CDR3-a | CAVNAGGYQKVTF | 177 |
| | FWR1-a | QKEVEQNSGPLSVPEGAIASLNCTYS | 15 |
| | FWR2-a | FFWYRQYSGKSPELIMF | 16 |
| | FWR3-a | KEDGRFTAQLNKASQYVSLLIRDSQPSDSATYL | 17 |
| | FWR4-a | GIGTKLQVIP | 178 |
| TCRB- | b-Variable | GATGCTGGAATCACCCAGAGCCCAAGACACAAGGTCACAGAGAC | 179 |
| 11 | nucleotide | AGGAACACCAGTGACTCTGAGATGTCATCAGACTGAGAACCACC | |
| | | GCTATATGTACTGGTATCGACAAGACCCGGGGCATGGGCTGAGG | |
| | | CTGATCCATTACTCATATGGTGTTAAAGATACTGACAAAGGAGAA | |
| | | GTCTCAGATGGCTATAGTGTCTCTAGATCAAAGACAGAGGATTTC | |
| | | CTCCTCACTCTGGAGTCCGCTACCAGCTCCCAGACATCTGTGTAC | |
| | | TTCTGTGCCATCAAGGGAGACGCGAGCAATCAGCCCCAGCATTTT | |
| | | GGTGATGGGACTCGACTCTCCATCCTAG | |
| | Variable-b | DAGITQSPRHKVTETGTPVTLRCHQTENHRYMYWYRQDPGHGLRLI | 180 |
| | | HYSYGVKDTDKGEVSDGYSVSRSKTEDFLLTLESATSSQTSVYFCAI | |
| | | KGDASNQPQHFGDGTRLSIL | |
| | CDR1-b | ENHRY | 181 |
| | CDR2-b | SYGVKD | 182 |
| | CDR3-b | CAIKGDASNQPQHF | 183 |
| | FWR1-b | DAGITQSPRHKVTETGTPVTLRCHQT | 184 |
| | FWR2-b | MYWYRQDPGHGLRLIHY | 185 |
| | FWR3-b | TDKGEVSDGYSVSRSKTEDFLLTLESATSSQTSVYF | 186 |
| | FWR4-b | GDGTRLSIL | 27 |
| TCRA- | a-Variable | AAACAGGAGGTGACGCAGATTCCTGCAGCTCTGAGTGTCCCAGA | 187 |
| 11 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGAGCTCTGACAGAGGCTCAACCCTGGGGAGGCTATACTT | |
| | | TGGAAGAGGAACTCAGTTGACTGTCTGGCCTG | |
| | Variable-a | KQEVTQIPAALSVPEGENLVLNCSFTDSAIYNLQWFRQDPGKGLTSL | 188 |
| | | LLIQSSQREQTSGRLNASLDKSSGRSTLYIAASQPGDSATYLCAVSSD | |
| | | RGSTLGRLYFGRGTQLTVWP | |
| | CDR1-a | DSAIYN | 118 |
| | CDR2-a | IQSSQRE | 119 |
| | CDR3-a | CAVSSDRGSTLGRLYF | 189 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GRGTQLTVWP | 190 |
| TCRB- | b-Variable | GGTGCTGTCGTCTCTCAACATCCGAGCTGGGTTATCTGTAAGAGT | 191 |
| 12 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGAGATATGGGACAGAAAATTAGCA | |
| | | ATCAGCCCCAGCATTTTGGTGATGGGACTCGACTCTCCATCCTAG | |
| | Variable-b | GAVVSQHPSWVICKSGTSVKIECRSLDFQATTMFWYRQFPKQSLML | 192 |
| | | MATSNEGSKATYEQGVEKDKFLINHASLTLSTLTVTSAHPEDSSFYIC | |
| | | SARDMGQKISNQPQHFGDGTRLSIL | |
| | CDR1-b | DFQATT | 79 |
| | CDR2-b | SNEGSKA | 80 |
| | CDR3-b | CSARDMGQKISNQPQHF | 193 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GDGTRLSIL | 27 |
| TCRA- | a-Variable | AAACAGGAGGTGACGCAGATTCCTGCAGCTCTGAGTGTCCCAGA | 194 |
| 12 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGGATGCAGGCAACATGCTCACCTTTGGAGGGGGAACAA | |
| | | GGTTAATGGTCAAACCCC | |
| | Variable-a | KQEVTQIPAALSVPEGENLVLNCSFTDSAIYNLQWFRQDPGKGLTSL | 195 |
| | | LLIQSSQREQTSGRLNASLDKSSGRSTLYIAASQPGDSATYLCAVDA | |
| | | GNMLTFGGGTRLMVKP | |
| | CDR1-a | DSAIYN | 118 |
| | CDR2-a | IQSSQRE | 119 |
| | CDR3-a | CAVDAGNMLTF | 196 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GGGTRLMVKP | 141 |
| TCRB- | b-Variable | GATGTGAAAGTAACCCAGAGCTCGAGATATCTAGTCAAAAGGAC | 197 |
| 13 | nucleotide | GGGAGAGAAAGTTTTTCTGGAATGTGTCCAGGATATGGACCATG | |
| | | AAAATATGTTCTGGTATCGACAAGACCCAGGTCTGGGGCTACGGC | |
| | | TGATCTATTTCTCATATGATGTTAAAATGAAAGAAAAAGGAGATA | |
| | | TTCCTGAGGGGTACAGTGTCTCTAGAGAGAAGAAGGAGCGCTTCT | |
| | | CCCTGATTCTGGAGTCCGCCAGCACCAACCAGACATCTATGTACC | |
| | | TCTGTGCCAGCGGCCCTGGGTATAGTTATAATTCACCCCTCCACTT | |
| | | TGGGAACGGGACCAGGCTCACTGTGACAG | |
| | Variable-b | DVKVTQSSRYLVKRTGEKVFLECVQDMDHENMFWYRQDPGLGLRL | 198 |
| | | IYFSYDVKMKEKGDIPEGYSVSREKKERFSLILESASTNQTSMYLCAS | |
| | | GPGYSYNSPLHFGNGTRLTVT | |
| | CDR1-b | MDHEN | 199 |
| | CDR2-b | SYDVKM | 200 |
| | CDR3-b | CASGPGYSYNSPLHF | 201 |
| | FWR1-b | DVKVTQSSRYLVKRTGEKVFLECVQD | 202 |
| | FWR2-b | MFWYRQDPGLGLRLIYF | 203 |
| | FWR3-b | KEKGDIPEGYSVSREKKERFSLILESASTNQTSMYL | 204 |
| | FWR4-b | GNGTRLTVT | 150 |
| TCRA- | a-Variable | GGAGAGAATGTGGAGCAGCATCCTTCAACCCTGAGTGTCCAGGA | 205 |
| 13 | nucleotide | GGGAGACAGCGCTGTTATCAAGTGTACTTATTCAGACAGTGCCTC | |
| | | AAACTACTTCCCTTGGTATAAGCAAGAACTTGGAAAAAGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGTCACTGGTTCTGCAAGGCAACTGACCTTTGGATCTGGGACAC | |
| | | AATTGACTGTTTTACCTG | |
| | Variable-a | GENVEQHPSTLSVQEGDSAVIKCTYSDSASNYFPWYKQELGKRPQLI | 206 |
| | | IDIRSNVGEKKDQRIA VTLNKTAKHFSLHITETQPEDSAVYFCVTGSA | |
| | | RQLTFGSGTQLTVLP | |
| | CDR1-a | DSASNY | 207 |
| | CDR2-a | IRSNVGE | 208 |
| | CDR3-a | CVTGSARQLTF | 209 |
| | FWR1-a | GENVEQHPSTLSVQEGDSAVIKCTYS | 210 |
| | FWR2-a | FPWYKQELGKRPQLIID | 211 |
| | FWR3-a | KKDQRIAVTLNKTAKHFSLHITETQPEDSAVYF | 212 |
| | FWR4-a | GSGTQLTVLP | 213 |
| TCRB- | b-Variable | GATGGTGGAATCACTCAGTCCCCAAAGTACCTGTTCAGAAAGGA | 214 |
| 14 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGCCCAGGGTGGCAAAGCAGATCCTATGGCTA | |
| | | CACCTTCGGTTCGGGGACCAGGTTAACCGTTGTAG | |
| | Variable-b | DGGITQSPKYLFRKEGQNVTLSCEQNLNHDAMYWYRQDPGQGLRLI | 215 |
| | | YYSQIVNDFQKGDIAEGYSVSREKKESFPLTVTSAQKNPTAFYLCAS | |
| | | SPGWQSRSYGYTFGSGTRLTVV | |
| | CDR1-b | LNHDA | 216 |
| | CDR2-b | SQIVND | 217 |
| | CDR3-b | CASSPGWQSRSYGYTF | 218 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GSGTRLTVV | 103 |
| TCRA- | a-Variable | CGGAAGGAGGTGGAGCAGGATCCTGGACCCTTCAATGTTCCAGA | 222 |
| 14 | nucleotide | GGGAGCCACTGTCGCTTTCAACTGTACTTACAGCAACAGTGCTTC | |
| | | TCAGTCTTTCTTCTGGTACAGACAGGATTGCAGGAAAGAACCTAA | |
| | | GTTGCTGATGTCCGTATACTCCAGTGGTAATGAAGATGGAAGGTT | |
| | | TACAGCACAGCTCAATAGAGCCAGCCAGTATATTTCCCTGCTCAT | |
| | | CAGAGACTCCAAGCTCAGTGATTCAGCCACCTACCTCTGTGTGGT | |
| | | GAACAATGCCAGACTCATGTTTGGAGATGGAACTCAGCTGGTGGT | |
| | | GAAGCCCA | |
| | Variable-a | RKEVEQDPGPFNVPEGATVAFNCTYSNSASQSFFWYRQDCRKEPKL | 223 |
| | | LMSVYSSGNEDGRFTAQLNRASQYISLLIRDSKLSDSATYLCVVNNA | |
| | | RLMFGDGTQLVVKP | |
| | CDR1-a | NSASQS | 224 |
| | CDR2-a | VYSSG | 225 |
| | CDR3-a | CVVNNARLMF | 226 |
| | FWR1-a | RKEVEQDPGPFNVPEGATVAFNCTYS | 227 |
| | FWR2-a | FFWYRQDCRKEPKLLMS | 228 |
| | FWR3-a | NEDGRFTAQLNRASQYISLLIRDSKLSDSATYL | 229 |
| | FWR4-a | GDGTQLVVKP | 230 |
| TCRB- | b-Variable | GATGCTGATGTTACCCAGACCCCAAGGAATAGGATCACAAAGAC | 231 |
| 15 | nucleotide | AGGAAAGAGGATTATGCTGGAATGTTCTCAGACTAAGGGTCATG | |
| | | ATAGAATGTACTGGTATCGACAAGACCCAGGACTGGGCCTACGG | |
| | | TTGATCTATTACTCCTTTGATGTCAAAGATATAAACAAAGGAGAG | |
| | | ATCTCTGATGGATACAGTGTCTCTCGACAGGCACAGGCTAAATTC | |
| | | TCCCTGTCCCTAGAGTCTGCCATCCCCAACCAGACAGCTCTTTACT | |
| | | TCTGTGCCACCAGTGATTTGTCCTCCCGGGACAGCCAAGAGACCC | |
| | | AGTACTTCGGGCCAGGCACGCGGCTCCTGGTGCTCG | |
| | Variable-b | DADVTQTPRNRITKTGKRIMLECSQTKGHDRMYWYRQDPGLGLRLI | 232 |
| | | YYSFDVKDINKGEISDGYSVSRQAQAKFSLSLESAIPNQTALYFCATS | |
| | | DLSSRDSQETQYFGPGTRLLVL | |
| | CDR1-b | KGHDR | 21 |
| | CDR2-b | SFDVKD | 22 |
| | CDR3-b | CATSDLSSRDSQETQYF | 233 |
| | FWR1-b | DADVTQTPRNRITKTGKRIMLECSQT | 24 |
| | FWR2-b | MYWYRQDPGLGLRLIYY | 25 |
| | FWR3-b | INKGEISDGYSVSRQAQAKFSLSLESAIPNQTALYF | 26 |
| | FWR4-b | GPGTRLLVL | 67 |
| TCRA- | a-Variable | AAGGACCAAGTGTTTCAGCCTTCCACAGTGGCATCTTCAGAGGGA | 234 |
| 15 | nucleotide | GCTGTGGTGGAAATCTTCTGTAATCACTCTGTGTCCAATGCTTAC | |
| | | AACTTCTTCTGGTACCTTCACTTCCCGGGATGTGCACCAAGACTC | |
| | | CTTGTTAAAGGCTCAAAGCCTTCTCAGCAGGGACGATACAACATG | |
| | | ACCTATGAACGGTTCTCTTCATCGCTGCTCATCCTCCAGGTGCGG | |
| | | GAGGCAGATGCTGCTGTTTACTACTGTGCTCCCGGGGCTGGCAAC | |
| | | AACCGTAAGCTGATTTGGGGATTGGGAACAAGCCTGGCAGTAAA | |
| | | TCCGA | |
| | Variable-a | KDQVFQPSTVASSEGAVVEIFCNHSVSNAYNFFWYLHFPGCAPRLLV | 235 |
| | | KGSKPSQQGRYNMTYERFSSSLLILQVREADAAVYYCAPGAGNNRK | |
| | | LIWGLGTSLAVNP | |
| | CDR1-a | VSNAYN | 236 |
| | CDR2-a | GSKP | 237 |
| | CDR3-a | CAPGAGNNRKLIW | 238 |
| | FWR1-a | KDQVFQPSTVASSEGAVVEIFCNHS | 239 |
| | FWR2-a | FFWYLHFPGCAPRLLVK | 240 |
| | FWR3-a | SQQGRYNMTYERFSSSLLILQVREADAAVYY | 241 |
| | FWR4-a | GLGTSLAVNP | 242 |
| TCRB- | b-Variable | GATGGTGGAATCACTCAGTCCCCAAAGTACCTGTTCAGAAAGGA | 243 |
| 16 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGTCGGACAGGTAACACTGAAGCTTTCTTTGG | |
| | | ACAAGGCACCAGACTCACAGTTGTAG | |
| | Variable-b | DGGITQSPKYLFRKEGQNVTLSCEQNLNHDAMYWYRQDPGQGLRLI | 244 |
| | | YYSQIVNDFQKGDIAEGYSVSREKKESFPLTVTSAQKNPTAFYLCAS | |
| | | SRTGNTEAFFGQGTRLTVV | |
| | CDR1-b | LNHDA | 216 |
| | CDR2-b | SQIVND | 217 |
| | CDR3-b | CASSRTGNTEAFF | 245 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GQGTRLTVV | 246 |
| TCRA- | a-Variable | AAACAGGAGGTGACGCAGATTCCTGCAGCTCTGAGTGTCCCAGA | 247 |
| 16 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGAGGCCTCCACGGGAATATGGAAACAAACTGGTCTTTGG | |
| | | CGCAGGAACCATTCTGAGAGTCAAGTCCT | |
| | Variable-a | KQEVTQIPAALSVPEGENLVLNCSFTDSAIYNLQWFRQDPGKGLTSL | 248 |
| | | LLIQSSQREQTSGRLNASLDKSSGRSTLYIAASQPGDSATYLCAVRPP | |
| | | REYGNKLVFGAGTILRVKS | |
| | CDR1-a | DSAIYN | 118 |
| | CDR2-a | IQSSQRE | 119 |
| | CDR3-a | CAVRPPREYGNKLVF | 249 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GAGTILRVKS | 18 |
| TCRB- | b-Variable | AATGCTGGTGTCACTCAGACCCCAAAATTCCAGGTCCTGAAGACA | 250 |
| 17 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTTACGGGGAGGGGAACGAGCAGTACTTCGGGC | |
| | | CGGGCACCAGGCTCACGGTCACAG | |
| | Variable-b | NAGVTQTPKFQVLKTGQSMTLQCAQDMNHEYMSWYRQDPGMGLR | 251 |
| | | LIHYSVGAGITDQGEVPNGYNVSRSTTEDFPLRLLSAAPSQTSVYFCA | |
| | | SSYGEGNEQYFGPGTRLTVT | |
| | CDR1-b | MNHEY | 252 |
| | CDR2-b | SVGAGI | 253 |
| | CDR3-b | CASSYGEGNEQYF | 254 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GPGTRLTVT | 85 |
| TCRA- | a-Variable | AAACAGGAGGTGACACAGATTCCTGCAGCTCTGAGTGTCCCAGA | 257 |
| 17 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGAGGGGAGAGGGCACGGGCAGGAGAGCACTTACTTTTG | |
| | | GGAGTGGAACAAGACTCCAAGTGCAACCAA | |
| | Variable-a | KQEVTQIPAALSVPEGENLVLNCSFTDSAIYNLQWFRQDPGKGLTSL | 258 |
| | | LLIQSSQREQTSGRLNASLDKSSGRSTLYIAASQPGDSATYLCAVRGE | |
| | | GTGRRALTFGSGTRLQVQP | |
| | CDR1-a | DSAIYN | 118 |
| | CDR2-a | IQSSQRE | 119 |
| | CDR3-a | CAVRGEGTGRRALTF | 259 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GSGTRLQVQP | 260 |
| TCRB- | b-Variable | GGTGCTGGAGTCTCCCAGACCCCCAGTAACAAGGTCACAGAGAA | 261 |
| 18 | nucleotide | GGGAAAATATGTAGAGCTCAGGTGTGATCCAATTTCAGGTCATAC | |
| | | TGCCCTTTACTGGTACCGACAAAGCCTGGGGCAGGGCCCAGAGTT | |
| | | TCTAATTTACTTCCAAGGCACGGGTGCGGCAGATGACTCAGGGCT | |
| | | GCCCAACGATCGGTTCTTTGCAGTCAGGCCTGAGGGATCCGTCTC | |
| | | TACTCTGAAGATCCAGCGCACAGAGCGGGGGGACTCAGCCGTGT | |
| | | ATCTCTGTGCCAGCAGCAATAGCGGGACATGGGGAGAGACCCAG | |
| | | TACTTCGGGCCAGGCACGCGGCTCCTGGTGCTCG | |
| | Variable-b | GAGVSQTPSNKVTEKGKYVELRCDPISGHTALYWYRQSLGQGPEFLI | 262 |
| | | YFQGTGAADDSGLPNDRFFAVRPEGSVSTLKIQRTERGDSAVYLCAS | |
| | | SNSGTWGETQYFGPGTRLLVL | |
| | CDR1-b | SGHTA | 263 |
| | CDR2-b | FQGTGA | 264 |
| | CDR3-b | CASSNSGTWGETQYF | 265 |
| | FWR1-b | GAGVSQTPSNKVTEKGKYVELRCDPI | 266 |
| | FWR2-b | LYWYRQSLGQGPEFLIY | 267 |
| | FWR3-b | ADDSGLPNDRFFAVRPEGSVSTLKIQRTERGDSAVYL | 268 |
| | FWR4-b | GPGTRLLVL | 67 |
| TCRA- | a-Variable | AAACAGGAGGTGACGCAGATTCCTGCAGCTCTGAGTGTCCCAGA | 269 |
| 18 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTCAGAGATAACTATGGTCAGAATTTTGTCTTTGGTCCCGG | |
| | | AACCAGATTGTCCGTGCTGCCCT | |
| | Variable-a | KQEVTQIPAALSVPEGENLVLNCSFTDSAIYNLQWFRQDPGKGLTSL | 270 |
| | | LLIQSSQREQTSGRLNASLDKSSGRSTLYIAASQPGDSATYLCAVRDN | |
| | | YGQNFVFGPGTRLSVLP | |
| | CDR1-a | DSAIYN | 118 |
| | CDR2-a | IQSSQRE | 119 |
| | CDR3-a | CAVRDNYGQNFVF | 271 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GPGTRLSVLP | 272 |
| TCRB- | b-Variable | GGTGCTGGAGTCTCCCAGTCTCCCAGGTACAAAGTCACAAAGAG | 273 |
| 19.1 | nucleotide | GGGACAGGATGTAGCTCTCAGGTGTGATCCAATTTCGGGTCATGT | |
| | | ATCCCTTTATTGGTACCGACAGGCCCTGGGGCAGGGCCCAGAGTT | |
| | | TCTGACTTACTTCAATTATGAAGCCCAACAAGACAAATCAGGGCT | |
| | | GCCCAATGATCGGTTCTCTGCAGAGAGGCCTGAGGGATCCATCTC | |
| | | CACTCTGACGATCCAGCGCACAGAGCAGCGGGACTCGGCCATGT | |
| | | ATCGCTGTGCCAGCAGCTTAGGGACCAGCGGGGACTACGAGCAG | |
| | | TACTTCGGGCCGGGCACCAGGCTCACGGTCACAG | |
| | Variable-b | GAGVSQSPRYKVTKRGQDVALRCDPISGHVSLYWYRQALGQGPEFL | 274 |
| | | TYFNYEAQQDKSGLPNDRFSAERPEGSISTLTIQRTEQRDSAMYRCA | |
| | | SSLGTSGDYEQYFGPGTRLTVT | |
| | CDR1-b | SGHVS | 275 |
| | CDR2-b | FNYEAQ | 276 |
| | CDR3-b | CASSLGTSGDYEQYF | 277 |
| | FWR1-b | GAGVSQSPRYKVTKRGQDVALRCDPI | 278 |
| | FWR2-b | LYWYRQALGQGPEFLTY | 279 |
| | FWR3-b | QDKSGLPNDRFSAERPEGSISTLTIQRTEQRDSAMYR | 280 |
| | FWR4-b | GPGTRLTVT | 85 |
| TCRB- | b-Variable | GGTGCTGTCGTCTCTCAACATCCGAGCTGGGTTATCTGTAAGAGT | 281 |
| 19.2 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGCATAGCGGGGGGACAAGAGACC | |
| | | CAGTACTTCGGGCCAGGCACGCGGCTCCTGGTGCTCG | |
| | Variable-b | GAVVSQHPSWVICKSGTSVKIECRSLDFQATTMFWYRQFPKQSLML | 282 |
| | | MATSNEGSKATYEQGVEKDKFLINHASLTLSTLTVTSAHPEDSSFYIC | |
| | | SASIAGGQETQYFGPGTRLLVL | |
| | CDR1-b | DFQATT | 79 |
| | CDR2-b | SNEGSKA | 80 |
| | CDR3-b | CSASIAGGQETQYF | 283 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLLVL | 67 |
| TCRA- | a-Variable | GCTCAGACAGTCACTCAGTCTCAACCAGAGATGTCTGTGCAGGAG | 284 |
| 19 | nucleotide | GCAGAGACCGTGACCCTGAGCTGCACATATGACACCAGTGAGAG | |
| | | TGATTATTATTTATTCTGGTACAAGCAGCCTCCCAGCAGGCAGAT | |
| | | GATTCTCGTTATTCGCCAAGAAGCTTATAAGCAACAGAATGCAAC | |
| | | AGAGAATCGTTTCTCTGTGAACTTCCAGAAAGCAGCCAAATCCTT | |
| | | CAGTCTCAAGATCTCAGACTCACAGCTGGGGGATGCCGCGATGTA | |
| | | TTTCTGTGCTTATAGGAGCGCGTCGTTTGGCTTTGGAAATGAGAA | |
| | | ATTAACCTTTGGGACTGGAACAAGACTCACCATCATACCCA | |
| | Variable-a | AQTVTQSQPEMSVQEAETVTLSCTYDTSESDYYLFWYKQPPSRQMI | 285 |
| | | LVIRQEAYKQQNATENRFSVNFQKAAKSFSLKISDSQLGDAAMYFC | |
| | | AYRSASFGFGNEKLTFGTGTRLTIIP | |
| | CDR1-a | TSESDYY | 286 |
| | CDR2-a | QEAYKQQN | 287 |
| | CDR3-a | CAYRSASFGFGNEKLTF | 288 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDAAMYF | 291 |
| | FWR4-a | GTGTRLTIIP | 292 |
| TCRB- | b-Variable | ACTGCTGGGATCACCCAGGCACCAACATCTCAGATCCTGGCAGCA | 293 |
| 20 | nucleotide | GGACGGAGCATGACACTGAGATGTACCCAGGATATGAGACATAA | |
| | | TGCCATGTACTGGTATAGACAAGATCTAGGACTGGGGCTAAGGCT | |
| | | CATCCATTATTCAAATACTGCAGGTACCACTGGCAAAGGAGAAGT | |
| | | CCCTGATGGTTATAGTGTCTCCAGAGCAAACACAGATGATTTCCC | |
| | | CCTCACGTTGGCGTCTGCTGTACCCTCTCAGACATCTGTGTACTTC | |
| | | TGTGCCAGCATCCCGAACAGGTTCTCTAACTATGGCTACACCTTC | |
| | | GGTTCGGGGACCAGGTTAACCGTTGTAG | |
| | Variable-b | TAGITQAPTSQILAAGRSMTLRCTQDMRHNAMYWYRQDLGLGLRLI | 294 |
| | | HYSNTAGTTGKGEVPDGYSVSRANTDDFPLTLASAVPSQTSVYFCAS | |
| | | IPNRFSNYGYTFGSGTRLTVV | |
| | CDR1-b | MRHNA | 295 |
| | CDR2-b | SNTAGT | 296 |
| | CDR3-b | CASIPNRFSNYGYTF | 297 |
| | FWR1-b | TAGITQAPTSQILAAGRSMTLRCTQD | 298 |
| | FWR2-b | MYWYRQDLGLGLRLIHY | 299 |
| | FWR3-b | TGKGEVPDGYSVSRANTDDFPLTLASAVPSQTSVYF | 300 |
| | FWR4-b | GSGTRLTVV | 103 |
| TCRA- | a-Variable | GACCAGCAAGTTAAGCAAAATTCACCATCCCTGAGCGTCCAGGA | 301 |
| 20 | nucleotide | AGGAAGAATTTCTATTCTGAACTGTGACTATACTAACAGCATGTT | |
| | | TGATTATTTCCTATGGTACAAAAAATACCCTGCTGAAGGTCCTAC | |
| | | ATTCCTGATATCTATAAGTTCCATTAAGGATAAAAATGAAGATGG | |
| | | AAGATTCACTGTCTTCTTAAACAAAAGTGCCAAGCACCTCTCTCT | |
| | | GCACATTGTGCCCTCCCAGCCTGGAGACTCTGCAGTGTACTTCTG | |
| | | TGCAGCAAGGGAGAATTCTGGTTCTGCAAGGCAACTGACCTTTGG | |
| | | ATCTGGGACACAATTGACTGTTTTACCTG | |
| | Variable-a | DQQVKQNSPSLSVQEGRISILNCDYTNSMFDYFLWYKKYPAEGPTFL | 302 |
| | | ISISSIKDKNEDGRFTVFLNKSAKHLSLHIVPSQPGDSA VYFCAARENS | |
| | | GSARQLTFGSGTQLTVLP | |
| | CDR1-a | NSMFDY | 303 |
| | CDR2-a | ISSIKDK | 304 |
| | CDR3-a | CAARENSGSARQLTF | 305 |
| | FWR1-a | DQQVKQNSPSLSVQEGRISILNCDYT | 306 |
| | FWR2-a | FLWYKKYPAEGPTFLIS | 307 |
| | FWR3-a | NEDGRFTVFLNKSAKHLSLHIVPSQPGDSAVYF | 308 |
| | FWR4-a | GSGTQLTVLP | 213 |
| TCRB- | b-Variable | GATGCTGATGTTACCCAGACCCCAAGGAATAGGATCACAAAGAC | 309 |
| 21 | nucleotide | AGGAAAGAGGATTATGCTGGAATGTTCTCAGACTAAGGGTCATG | |
| | | ATAGAATGTACTGGTATCGACAAGACCCAGGACTGGGCCTACGG | |
| | | TTGATCTATTACTCCTTTGATGTCAAAGATATAAACAAAGGAGAG | |
| | | ATCTCTGATGGATACAGTGTCTCTCGACAGGCACAGGCTAAATTC | |
| | | TCCCTGTCCCTAGAGTCTGCCATCCCCAACCAGACAGCTCTTTACT | |
| | | TCTGTGCCACCAGTGACAACAAAGAGCAGTACTTCGGGCCGGGC | |
| | | ACCAGGCTCACGGTCACAG | |
| | Variable-b | DADVTQTPRNRITKTGKRIMLECSQTKGHDRMYWYRQDPGLGLRLI | 310 |
| | | YYSFDVKDINKGEISDGYSVSRQAQAKFSLSLESAIPNQTALYFCATS | |
| | | DNKEQYFGPGTRLTVT | |
| | CDR1-b | KGHDR | 21 |
| | CDR2-b | SFDVKD | 22 |
| | CDR3-b | CATSDNKEQYF | 311 |
| | FWR1-b | DADVTQTPRNRITKTGKRIMLECSQT | 24 |
| | FWR2-b | MYWYRQDPGLGLRLIYY | 25 |
| | FWR3-b | INKGEISDGYSVSRQAQAKFSLSLESAIPNQTALYF | 26 |
| | FWR4-b | GPGTRLTVT | 85 |
| TCRA- | a-Variable | GGAGAGGATGTGGAGCAGAGTCTTTTCCTGAGTGTCCGAGAGGG | 312 |
| 21 | nucleotide | AGACAGCTCCGTTATAAACTGCACTTACACAGACAGCTCCTCCAC | |
| | | CTACTTATACTGGTATAAGCAAGAACCTGGAGCAGGTCTCCAGTT | |
| | | GCTGACGTATATTTTTTCAAATATGGACATGAAACAAGACCAAAG | |
| | | ACTCACTGTTCTATTGAATAAAAAGGATAAACATCTGTCTCTGCG | |
| | | CATTGCAGACACCCAGACTGGGGACTCAGCTATCTACTTCTGTGC | |
| | | AGAGGACCGATACAGCACCCTCACCTTTGGGAAGGGGACTATGC | |
| | | TTCTAGTCTCTCCAG | |
| | Variable-a | GEDVEQSLFLSVREGDSSVINCTYTDSSSTYLYWYKQEPGAGLQLLT | 313 |
| | | YIFSNMDMKQDQRLTVLLNKKDKHLSLRIADTQTGDSAIYFCAEDR | |
| | | YSTLTFGKGTMLLVSP | |
| | CDR1-a | DSSSTY | 314 |
| | CDR2-a | IFSNMDM | 315 |
| | CDR3-a | CAEDRYSTLTF | 316 |
| | FWR1-a | GEDVEQSLFLSVREGDSSVINCTYT | 317 |
| | FWR2-a | LYWYKQEPGAGLQLLTY | 318 |
| | FWR3-a | KQDQRLTVLLNKKDKHLSLRIADTQTGDSAIYF | 319 |
| | FWR4-a | GKGTMLLVSP | 320 |
| TCRB- | b-Variable | GATGGTGGAATCACTCAGTCCCCAAAGTACCTGTTCAGAAAGGA | 321 |
| 22 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTGAGGCAGATACGCAGTATTTTGGCCCAGGCAC | |
| | | CCGGCTGACAGTGCTCG | |
| | Variable-b | DGGITQSPKYLFRKEGQNVTLSCEQNLNHDAMYWYRQDPGQGLRLI | 322 |
| | | YYSQIVNDFQKGDIAEGYSVSREKKESFPLTVTSAQKNPTAFYLCAS | |
| | | EADTQYFGPGTRLTVL | |
| | CDR1-b | LNHDA | 216 |
| | CDR2-b | SQIVND | 217 |
| | CDR3-b | CASEADTQYF | 323 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVL | 9 |
| TCRA- | a-Variable | CAGAAGGAGGTGGAGCAGAATTCTGGACCCCTCAGTGTTCCAGA | 324 |
| 22 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTTCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAACAGGGGAAGGGATAAATCTGTGAACACCGGTAACCAG | |
| | | TTCTATTTTGGGACAGGGACAAGTTTGACGGTCATTCCAA | |
| | Variable-a | QKEVEQNSGPLSVPEGAIASLNCTYSDRGSQSFFWYRQYSGKSPELI | 325 |
| | | MFIYSNGDKEDGRFTAQLNKASQYVSLLIRDSQPSDSATYLCAVNRG | |
| | | RDKSVNTGNQFYFGTGTSLTVIP | |
| | CDR1-a | DRGSQS | 12 |
| | CDR2-a | IYSNGD | 13 |
| | CDR3-a | CAVNRGRDKSVNTGNQFYF | 326 |
| | FWR1-a | QKEVEQNSGPLSVPEGAIASLNCTYS | 15 |
| | FWR2-a | FFWYRQYSGKSPELIMF | 16 |
| | FWR3-a | KEDGRFTAQLNKASQYVSLLIRDSQPSDSATYL | 17 |
| | FWR4-a | GTGTSLTVIP | 94 |
| TCRB- | b-Variable | GATGGTGGAATCACTCAGTCCCCAAAGTACCTGTTCAGAAAGGA | 327 |
| 23.1 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTGAGGCAGATACGCAGTATTTTGGCCCAGGCAC | |
| | | CCGGCTGACAGTGCTCG | |
| | Variable-b | DGGITQSPKYLFRKEGQNVTLSCEQNLNHDAMYWYRQDPGQGLRLI | 328 |
| | | YYSQIVNDFQKGDIAEGYSVSREKKESFPLTVTSAQKNPTAFYLCAS | |
| | | EADTQYFGPGTRLTVL | |
| | CDR1-b | LNHDA | 216 |
| | CDR2-b | SQIVND | 217 |
| | CDR3-b | CASEADTQYF | 323 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVL | 9 |
| TCRB- | b-Variable | GACGCTGGAGTCACCCAAAGTCCCACACACCTGATCAAAACGAG | 329 |
| 23.2 | nucleotide | AGGACAGCAAGTGACTCTGAGATGCTCTCCTAAGTCTGGGCATGA | |
| | | CACTGTGTCCTGGTACCAACAGGCCCTGGGTCAGGGGCCCCAGTT | |
| | | TATCTTTCAGTATTATGAGGAGGAAGAGAGACAGAGAGGCAACT | |
| | | TCCCTGATCGATTCTCAGGTCACCAGTTCCCTAACTATAGCTCTGA | |
| | | GCTGAATGTGAACGCCTTGTTGCTGGGGGACTCGGCCCTCTATCT | |
| | | CTGTGCCAGCAGGGGACAGGAGGAGGGCTACACCTTCGGTTCGG | |
| | | GGACCAGGTTAACCGTTGTAG | |
| | Variable-b | DAGVTQSPTHLIKTRGQQVTLRCSPKSGHDTVSWYQQALGQGPQFIF | 330 |
| | | QYYEEEERQRGNFPDRFSGHQFPNYSSELNVNALLLGDSALYLCASR | |
| | | GQEEGYTFGSGTRLTVV | |
| | CDR1-b | SGHDT | 331 |
| | CDR2-b | YYEEEE | 332 |
| | CDR3-b | CASRGQEEGYTF | 333 |
| | FWR1-b | DAGVTQSPTHLIKTRGQQVTLRCSPK | 334 |
| | FWR2-b | VSWYQQALGQGPQFIFQ | 131 |
| | FWR3-b | RQRGNFPDRFSGHQFPNYSSELNVNALLLGDSALYL | 335 |
| | FWR4-b | GSGTRLTVV | 103 |
| TCRA- | a-Variable | GACCAGCAAGTTAAGCAAAATTCACCATCCCTGAGCGTCCAGGA | 336 |
| 23 | nucleotide | AGGAAGAATTTCTATTCTGAACTGTGACTATACTAACAGCATGTT | |
| | | TGATTATTTCCTATGGTACAAAAAATACCCTGCTGAAGGTCCTAC | |
| | | ATTCCTGATATCTATAAGTTCCATTAAGGATAAAAATGAAGATGG | |
| | | AAGATTCACTGTCTTCTTAAACAAAAGTGCCAAGCACCTCTCTCT | |
| | | GCACATTGTGCCCTCCCAGCCTGGAGACTCTGCAGTGTACTTCTG | |
| | | TGCAGCAACCCCCGGCCAGGGCGGATCTGAAAAGCTGGTCTTTG | |
| | | GAAAGGGAACGAAACTGACAGTAAACCCAT | |
| | Variable-a | DQQVKQNSPSLSVQEGRISILNCDYTNSMFDYFLWYKKYPAEGPTFL | 337 |
| | | ISISSIKDKNEDGRFTVFLNKSAKHLSLHIVPSQPGDSAVYFCAATPG | |
| | | QGGSEKLVFGKGTKLTVNP | |
| | CDR1-a | NSMFDY | 303 |
| | CDR2-a | ISSIKDK | 304 |
| | CDR3-a | CAATPGQGGSEKLVF | 338 |
| | FWR1-a | DQQVKQNSPSLSVQEGRISILNCDYT | 306 |
| | FWR2-a | FLWYKKYPAEGPTFLIS | 307 |
| | FWR3-a | NEDGRFTVFLNKSAKHLSLHIVPSQPGDSAVYF | 308 |
| | FWR4-a | GKGTKLTVNP | 339 |
| TCRB- | b-Variable | GACACAGCTGTTTCCCAGACTCCAAAATACCTGGTCACACAGATG | 340 |
| 24 | nucleotide | GGAAACGACAAGTCCATTAAATGTGAACAAAATCTGGGCCATGA | |
| | | TACTATGTATTGGTATAAACAGGACTCTAAGAAATTTCTGAAGAT | |
| | | AATGTTTAGCTACAATAATAAGGAGCTCATTATAAATGAAACAGT | |
| | | TCCAAATCGCTTCTCACCTAAATCTCCAGACAAAGCTCACTTAAA | |
| | | TCTTCACATCAATTCCCTGGAGCTTGGTGACTCTGCTGTGTATTTC | |
| | | TGTGCCAGCAGCCAAGTGGCGGGGGGGAGCGATGAGCAGTTCTT | |
| | | CGGGCCAGGGACACGGCTCACCGTGCTAG | |
| | Variable-b | DTAVSQTPKYLVTQMGNDKSIKCEQNLGHDTMYWYKQDSKKFLKI | 341 |
| | | MFSYNNKELIINETVPNRFSPKSPDKAHLNLHINSLELGDSAVYFCAS | |
| | | SQVAGGSDEQFFGPGTRLTVL | |
| | CDR1-b | LGHDT | 342 |
| | CDR2-b | YNNKEL | 343 |
| | CDR3-b | CASSQVAGGSDEQFF | 344 |
| | FWR1-b | DTAVSQTPKYLVTQMGNDKSIKCEQN | 345 |
| | FWR2-b | MYWYKQDSKKFLKIMFS | 346 |
| | FWR3-b | IINETVPNRFSPKSPDKAHLNLHINSLELGDSAVYF | 347 |
| | FWR4-b | GPGTRLTVL | 9 |
| TCRA- | a-Variable | GGACAAAGCCTTGAGCAGCCCTCTGAAGTGACAGCTGTGGAAGG | 348 |
| 24 | nucleotide | AGCCATTGTCCAGATAAACTGCACGTACCAGACATCTGGGTTTTA | |
| | | TGGGCTGTCCTGGTACCAGCAACATGATGGCGGAGCACCCACATT | |
| | | TCTTTCTTACAATGCTCTGGATGGTTTGGAGGAGACAGGTCGTTTT | |
| | | TCTTCATTCCTTAGTCGCTCTGATAGTTATGGTTACCTCCTTCTAC | |
| | | AGGAGCTCCAGATGAAAGACTCTGCCTCTTACTTCTGCGCTGAAC | |
| | | ATGCAGGTAGCAACTATCAGTTAATCTGGGGCGCTGGGACCAAG | |
| | | CTAATTATAAAGCCAG | |
| | Variable-a | GQSLEQPSEVTAVEGAIVQINCTYQTSGFYGLSWYQQHDGGAPTFLS | 349 |
| | | YNALDGLEETGRESSFLSRSDSYGYLLLQELQMKDSASYFCAEHAGS | |
| | | NYQLIWGAGTKLIIKP | |
| | CDR1-a | TSGFYG | 350 |
| | CDR2-a | NALDGL | 351 |
| | CDR3-a | CAEHAGSNYQLIW | 352 |
| | FWR1-a | GQSLEQPSEVTAVEGAIVQINCTYQ | 353 |
| | FWR2-a | LSWYQQHDGGAPTFLSY | 354 |
| | FWR3-a | EETGRESSFLSRSDSYGYLLLQELQMKDSASYF | 355 |
| | FWR4-a | GAGTKLIIKP | 356 |
| TCRB- | b-Variable | AATGCCGGCGTCATGCAGAACCCAAGACACCTGGTCAGGAGGAG | 357 |
| 25 | nucleotide | GGGACAGGAGGCAAGACTGAGATGCAGCCCAATGAAAGGACAC | |
| | | AGTCATGTTTACTGGTATCGGCAGCTCCCAGAGGAAGGTCTGAAA | |
| | | TTCATGGTTTATCTCCAGAAAGAAAATATCATAGATGAGTCAGGA | |
| | | ATGCCAAAGGAACGATTTTCTGCTGAATTTCCCAAAGAGGGCCCC | |
| | | AGCATCCTGAGGATCCAGCAGGTAGTGCGAGGAGATTCGGCAGC | |
| | | TTATTTCTGTGCCAGCTCACCGACCGGGACAGGGGATAACACCGG | |
| | | GGAGCTGTTTTTTGGAGAAGGCTCTAGGCTGACCGTACTGG | |
| | Variable-b | NAGVMQNPRHLVRRRGQEARLRCSPMKGHSHVYWYRQLPEEGLKF | 358 |
| | | MVYLQKENIIDESGMPKERFSAEFPKEGPSILRIQQVVRGDSAAYFCA | |
| | | SSPTGTGDNTGELFFGEGSRLTVL | |
| | CDR1-b | KGHSH | 144 |
| | CDR2-b | LQKENI | 145 |
| | CDR3-b | CASSPTGTGDNTGELFF | 359 |
| | FWR1-b | NAGVMQNPRHLVRRRGQEARLRCSPM | 147 |
| | FWR2-b | VYWYRQLPEEGLKFMVY | 148 |
| | FWR3-b | IDESGMPKERFSAEFPKEGPSILRIQQVVRGDSAAYF | 149 |
| | FWR4-b | GEGSRLTVL | 63 |
| TCRA- | a-Variable | GACCAGCAAGTTAAGCAAAATTCACCATCCCTGAGCGTCCAGGA | 360 |
| 25 | nucleotide | AGGAAGAATTTCTATTCTGAACTGTGACTATACTAACAGCATGTT | |
| | | TGATTATTTCCTATGGTACAAAAAATACCCTGCTGAAGGTCCTAC | |
| | | ATTCCTGATATCTATAAGTTCCATTAAGGATAAAAATGAAGATGG | |
| | | AAGATTCACTGTCTTCTTAAACAAAAGTGCCAAGCACCTCTCTCT | |
| | | GCACATTGTGCCCTCCCAGCCTGGAGACTCTGCAGTGTACTTCTG | |
| | | TGCAGCAAGCGAGGGCCCGGGAGGAGGTGCTGACGGACTCACCT | |
| | | TTGGCAAAGGGACTCATCTAATCATCCAGCCCT | |
| | Variable-a | DQQVKQNSPSLSVQEGRISILNCDYTNSMFDYFLWYKKYPAEGPTFL | 361 |
| | | ISISSIKDKNEDGRFTVFLNKSAKHLSLHIVPSQPGDSAVYFCAASEGP | |
| | | GGGADGLTFGKGTHLIIQP | |
| | CDR1-a | NSMFDY | 303 |
| | CDR2-a | ISSIKDK | 304 |
| | CDR3-a | CAASEGPGGGADGLTF | 362 |
| | FWR1-a | DQQVKQNSPSLSVQEGRISILNCDYT | 306 |
| | FWR2-a | FLWYKKYPAEGPTFLIS | 307 |
| | FWR3-a | NEDGRFTVFLNKSAKHLSLHIVPSQPGDSAVYF | 308 |
| | FWR4-a | GKGTHLIIQP | 363 |
| TCRB- | b-Variable | GAAGCCCAAGTGACCCAGAACCCAAGATACCTCATCACAGTGAC | 364 |
| 26 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTTTTTCGGGACAGGGAATCGAAGCTTTCTTTG | |
| | | GACAAGGCACCAGACTCACAGTTGTAG | |
| | Variable-b | EAQVTQNPRYLITVTGKKLTVTCSQNMNHEYMSWYRQDPGLGLRQ | 365 |
| | | IYYSMNVEVTDKGDVPEGYKVSRKEKRNFPLILESPSPNQTSLYFCA | |
| | | SSFSGQGIEAFFGQGTRLTVV | |
| | CDR1-b | MNHEY | 252 |
| | CDR2-b | SMNVEV | 366 |
| | CDR3-b | CASSFSGQGIEAFF | 367 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GQGTRLTVV | 246 |
| TCRA- | a-Variable | GGAGAGAATGTGGAGCAGCATCCTTCAACCCTGAGTGTCCAGGA | 371 |
| 26 | nucleotide | GGGAGACAGCGCTGTTATCAAGTGTACTTATTCAGACAGTGCCTC | |
| | | AAACTACTTCCCTTGGTATAAGCAAGAACTTGGAAAAGGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCAGTTAACTCTGGTTCTGCAAGGCAACTGACCTTTGGAT | |
| | | CTGGGACACAATTGACTGTTTTACCTG | |
| | Variable-a | GENVEQHPSTLSVQEGDSAVIKCTYSDSASNYFPWYKQELGKGPQLI | 372 |
| | | IDIRSNVGEKKDQRIA VTLNKTAKHFSLHITETQPEDSAVYFCAAVNS | |
| | | GSARQLTFGSGTQLTVLP | |
| | CDR1-a | DSASNY | 207 |
| | CDR2-a | IRSNVGE | 208 |
| | CDR3-a | CAAVNSGSARQLTF | 373 |
| | FWR1-a | GENVEQHPSTLSVQEGDSAVIKCTYS | 210 |
| | FWR2-a | FPWYKQELGKGPQLIID | 374 |
| | FWR3-a | KKDQRIAVTLNKTAKHFSLHITETQPEDSAVYF | 212 |
| | FWR4-a | GSGTQLTVLP | 213 |
| TCRB- | b-Variable | GATGGTGGAATCACTCAGTCCCCAAAGTACCTGTTCAGAAAGGA | 375 |
| 27 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTACCCTGACTAGCGGAGACTACAATGAGCAGTT | |
| | | CTTCGGGCCAGGGACACGGCTCACCGTGCTAG | |
| | Variable-b | DGGITQSPKYLFRKEGQNVTLSCEQNLNHDAMYWYRQDPGQGLRLI | 376 |
| | | YYSQIVNDFQKGDIAEGYSVSREKKESFPLTVTSAQKNPTAFYLCAS | |
| | | TLTSGDYNEQFFGPGTRLTVL | |
| | CDR1-b | LNHDA | 216 |
| | CDR2-b | SQIVND | 217 |
| | CDR3-b | CASTLTSGDYNEQFF | 377 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVL | 9 |
| TCRA- | a-Variable | CGGAAGGAGGTGGAGCAGGATCCTGGACCCTTCAATGTTCCAGA | 378 |
| 27 | nucleotide | GGGAGCCACTGTCGCTTTCAACTGTACTTACAGCAACAGTGCTTC | |
| | | TCAGTCTTTCTTCTGGTACAGACAGGATTGCAGGAAAGAACCTAA | |
| | | GTTGCTGATGTCCGTATACTCCAGTGGTAATGAAGATGGAAGGTT | |
| | | TACAGCACAGCTCAATAGAGCCAGCCAGTATATTTCCCTGCTCAT | |
| | | CAGAGACTCCAAGCTCAGTGATTCAGCCACCTACCTCTGTGTGGT | |
| | | GGATTTTTATAACACCGACAAGCTCATCTTTGGGACTGGGACCAG | |
| | | ATTACAAGTCTTTCCAA | |
| | Variable-a | RKEVEQDPGPFNVPEGATVAFNCTYSNSASQSFFWYRQDCRKEPKL | 379 |
| | | LMSVYSSGNEDGRFTAQLNRASQYISLLIRDSKLSDSATYLCVVDFY | |
| | | NTDKLIFGTGTRLQVFP | |
| | CDR1-a | NSASQS | 224 |
| | CDR2-a | VYSSG | 225 |
| | CDR3-a | CVVDFYNTDKLIF | 380 |
| | FWR1-a | RKEVEQDPGPFNVPEGATVAFNCTYS | 227 |
| | FWR2-a | FFWYRQDCRKEPKLLMS | 228 |
| | FWR3-a | NEDGRFTAQLNRASQYISLLIRDSKLSDSATYL | 229 |
| | FWR4-a | GTGTRLQVFP | 36 |
| TCRB- | b-Variable | GATTCTGGAGTCACACAAACCCCAAAGCACCTGATCACAGCAAC | 381 |
| 28 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCCTCAGTAAGGGGGCGGGAACCGGGGAGCTGT | |
| | | TTTTTGGAGAAGGCTCTAGGCTGACCGTACTGG | |
| | Variable-b | DSGVTQTPKHLITATGQRVTLRCSPRSGDLSVYWYQQSLDQGLQFLI | 382 |
| | | QYYNGEERAKGNILERFSAQQFPDLHSELNLSSLELGDSALYFCASSL | |
| | | SKGAGTGELFFGEGSRLTVL | |
| | CDR1-b | SGDLS | 383 |
| | CDR2-b | YYNGEE | 384 |
| | CDR3-b | CASSLSKGAGTGELFF | 385 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GEGSRLTVL | 63 |
| TCRA- | a-Variable | GGAGAGAATGTGGAGCAGCATCCTTCAACCCTGAGTGTCCAGGA | 389 |
| 28 | nucleotide | GGGAGACAGCGCTGTTATCAAGTGTACTTATTCAGACAGTGCCTC | |
| | | AAACTACTTCCCTTGGTATAAGCAAGAACTTGGAAAAAGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCAACAGATGGCCAGAAGCTGCTCTTTGCAAGGGGGACC | |
| | | ATGTTAAAGGTGGATCTTA | |
| | Variable-a | GENVEQHPSTLSVQEGDSAVIKCTYSDSASNYFPWYKQELGKRPQLI | 390 |
| | | IDIRSNVGEKKDQRIA VTLNKTAKHFSLHITETQPEDSAVYFCAATDG | |
| | | QKLLFARGTMLKVDL | |
| | CDR1-a | DSASNY | 207 |
| | CDR2-a | IRSNVGE | 208 |
| | CDR3-a | CAATDGQKLLF | 391 |
| | FWR1-a | GENVEQHPSTLSVQEGDSAVIKCTYS | 210 |
| | FWR2-a | FPWYKQELGKRPQLIID | 211 |
| | FWR3-a | KKDQRIAVTLNKTAKHFSLHITETQPEDSAVYF | 212 |
| | FWR4-a | ARGTMLKVDL | 392 |
| TCRB- | b-Variable | TCTCAGACTATTCATCAATGGCCAGCGACCCTGGTGCAGCCTGTG | 393 |
| 29 | nucleotide | GGCAGCCCGCTCTCTCTGGAGTGCACTGTGGAGGGAACATCAAA | |
| | | CCCCAACCTATACTGGTACCGACAGGCTGCAGGCAGGGGCCTCC | |
| | | AGCTGCTCTTCTACTCCGTTGGTATTGGCCAGATCAGCTCTGAGG | |
| | | TGCCCCAGAATCTCTCAGCCTCCAGACCCCAGGACCGGCAGTTCA | |
| | | TCCTGAGTTCTAAGAAGCTCCTTCTCAGTGACTCTGGCTTCTATCT | |
| | | CTGTGCCTGGGAGGGGGTAAGGGGCTACACCTTCGGTTCGGGGA | |
| | | CCAGGTTAACCGTTGTAG | |
| | Variable-b | SQTIHQWPATLVQPVGSPLSLECTVEGTSNPNLYWYRQAAGRGLQL | 394 |
| | | LFYSVGIGQISSEVPQNLSASRPQDRQFILSSKKLLLSDSGFYLCAWE | |
| | | GVRGYTFGSGTRLTVV | |
| | CDR1-b | GTSNPN | 395 |
| | CDR2-b | SVGIG | 396 |
| | CDR3-b | CAWEGVRGYTF | 397 |
| | FWR1-b | SQTIHQWPATLVQPVGSPLSLECTVE | 398 |
| | FWR2-b | LYWYRQAAGRGLQLLFY | 399 |
| | FWR3-b | QISSEVPQNLSASRPQDRQFILSSKKLLLSDSGFYL | 400 |
| | FWR4-b | GSGTRLTVV | 103 |
| TCRA- | a-Variable | GCCCAGAAGATAACTCAAACCCAACCAGGAATGTTCGTGCAGGA | 401 |
| 29 | nucleotide | AAAGGAGGCTGTGACTCTGGACTGCACATATGACACCAGTGATC | |
| | | AAAGTTATGGTCTCTTCTGGTACAAGCAGCCCAGCAGTGGGGAA | |
| | | ATGATTTTTCTTATTTATCAGGGGTCTTATGACGAGCAAAATGCA | |
| | | ACAGAAGGTCGCTACTCATTGAATTTCCAGAAGGCAAGAAAATC | |
| | | CGCCAACCTTGTCATCTCCGCTTCACAACTGGGGGACTCAGCAAT | |
| | | GTATTTCTGTGCAATGACCTCTTCTTATAACCAGGGAGGAAAGCT | |
| | | TATCTTCGGACAGGGAACGGAGTTATCTGTGAAACCCA | |
| | Variable-a | AQKITQTQPGMFVQEKEAVTLDCTYDTSDQSYGLFWYKQPSSGEMI | 402 |
| | | FLIYQGSYDEQNATEGRYSLNFQKARKSANLVISASQLGDSAMYFC | |
| | | AMTSSYNQGGKLIFGQGTELSVKP | |
| | CDR1-a | TSDQSYG | 403 |
| | CDR2-a | QGSYDEQN | 404 |
| | CDR3-a | CAMTSSYNQGGKLIF | 405 |
| | FWR1-a | AQKITQTQPGMFVQEKEAVTLDCTYD | 406 |
| | FWR2-a | LFWYKQPSSGEMIFLIY | 407 |
| | FWR3-a | ATEGRYSLNFQKARKSANLVISASQLGDSAMYF | 408 |
| | FWR4-a | GQGTELSVKP | 409 |
| TCRB- | b-Variable | GGTGCTGTCGTCTCTCAACATCCGAGCTGGGTTATCTGTAAGAGT | 410 |
| 30 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGAGAGGGGACAGTCCGAGCTTTCT | |
| | | TTGGACAAGGCACCAGACTCACAGTTGTAG | |
| | Variable-b | GAVVSQHPSWVICKSGTSVKIECRSLDFQATTMFWYRQFPKQSLML | 411 |
| | | MATSNEGSKATYEQGVEKDKFLINHASLTLSTLTVTSAHPEDSSFYIC | |
| | | SAREGTVRAFFGQGTRLTVV | |
| | CDR1-b | DFQATT | 79 |
| | CDR2-b | SNEGSKA | 80 |
| | CDR3-b | CSAREGTVRAFF | 412 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GQGTRLTVV | 246 |
| TCRA- | a-Variable | GAAGACCAGGTGACGCAGAGTCCCGAGGCCCTGAGACTCCAGGA | 413 |
| 30 | nucleotide | GGGAGAGAGTAGCAGTCTCAACTGCAGTTACACAGTCAGCGGTT | |
| | | TAAGAGGGCTGTTCTGGTATAGGCAAGATCCTGGGAAAGGCCCT | |
| | | GAATTCCTCTTCACCCTGTATTCAGCTGGGGAAGAAAAGGAGAA | |
| | | AGAAAGGCTAAAAGCCACATTAACAAAGAAGGAAAGCTTTCTGC | |
| | | ACATCACAGCCCCTAAACCTGAAGACTCAGCCACTTATCTCTGTG | |
| | | CTGTGCGTCTTTCTTCTGGTTCTGCAAGGCAACTGACCTTTGGATC | |
| | | TGGGACACAATTGACTGTTTTACCTG | |
| | Variable-a | EDQVTQSPEALRLQEGESSSLNCSYTVSGLRGLFWYRQDPGKGPEFL | 414 |
| | | FTLYSAGEEKEKERLKATLTKKESFLHITAPKPEDSATYLCAVRLSSG | |
| | | SARQLTFGSGTQLTVLP | |
| | CDR1-a | VSGLRG | 415 |
| | CDR2-a | LYSAGEE | 416 |
| | CDR3-a | CAVRLSSGSARQLTF | 417 |
| | FWR1-a | EDQVTQSPEALRLQEGESSSLNCSYT | 418 |
| | FWR2-a | LFWYRQDPGKGPEFLFT | 419 |
| | FWR3-a | KEKERLKATLTKKESFLHITAPKPEDSATYL | 420 |
| | FWR4-a | GSGTQLTVLP | 213 |
| TCRB- | b-Variable | GATTCTGGAGTCACACAAACCCCAAAGCACCTGATCACAGCAAC | 421 |
| 31 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCACTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCGACTCTCATACGCAGTATTTTGGCCCAGGCAC | |
| | | CCGGCTGACAGTGCTCG | |
| | Variable-b | DSGVTQTPKHLITATGQRVTLRCSPRSGDLSVYWYQQSLDQGLQFLI | 422 |
| | | HYYNGEERAKGNILERFSAQQFPDLHSELNLSSLELGDSALYFCASS | |
| | | DSHTQYFGPGTRLTVL | |
| | CDR1-b | SGDLS | 383 |
| | CDR2-b | YYNGEE | 384 |
| | CDR3-b | CASSDSHTQYF | 423 |
| | FWR1-b | DSGVTQTPKHLITATGQRVTLRCSPR | 386 |
| | FWR2-b | VYWYQQSLDQGLQFLIH | 424 |
| | FWR3-b | RAKGNILERFSAQQFPDLHSELNLSSLELGDSALYF | 388 |
| | FWR4-b | GPGTRLTVL | 9 |
| TCRA- | a-Variable | GCTCAGTCAGTGGCTCAGCCGGAAGATCAGGTCAACGTTGCTGA | 425 |
| 31 | nucleotide | AGGGAATCCTCTGACTGTGAAATGCACCTATTCAGTCTCTGGAAA | |
| | | CCCTTATCTTTTTTGGTATGTTCAATACCCCAACCGAGGCCTCCAG | |
| | | TTCCTTCTGAAATACATCACAGGGGATAACCTGGTTAAAGGCAGC | |
| | | TATGGCTTTGAAGCTGAATTTAACAAGAGCCAAACCTCCTTCCAC | |
| | | CTGAAGAAACCATCTGCCCTTGTGAGCGACTCCGCTTTGTACTTC | |
| | | TGTGCTGTGAGAGACTCCCCATATTCAGGAGGAGGTGCTGACGG | |
| | | ACTCACCTTTGGCAAAGGGACTCATCTAATCATCCAGCCCT | |
| | Variable-a | AQSVAQPEDQVNVAEGNPLTVKCTYSVSGNPYLFWYVQYPNRGLQ | 426 |
| | | FLLKYITGDNLVKGSYGFEAEFNKSQTSFHLKKPSALVSDSALYFCA | |
| | | VRDSPYSGGGADGLTFGKGTHLIIQP | |
| | CDR1-a | VSGNPY | 427 |
| | CDR2-a | YITGDNLV | 428 |
| | CDR3-a | CAVRDSPYSGGGADGLTF | 429 |
| | FWR1-a | AQSVAQPEDQVNVAEGNPLTVKCTYS | 430 |
| | FWR2-a | LFWYVQYPNRGLQFLLK | 431 |
| | FWR3-a | KGSYGFEAEFNKSQTSFHLKKPSALVSDSALYF | 432 |
| | FWR4-a | GKGTHLIIQP | 363 |
| TCRB- | b-Variable | AGTGCTGTCATCTCTCAAAAGCCAAGCAGGGATATCTGTCAACGT | 433 |
| 32 | nucleotide | GGAACCTCCCTGACGATCCAGTGTCAAGTCGATAGCCAAGTCACC | |
| | | ATGATGTTCTGGTACCGTCAGCAACCTGGACAGAGCCTGACACTG | |
| | | ATCGCAACTGCAAATCAGGGCTCTGAGGCCACATATGAGAGTGG | |
| | | ATTTGTCATTGACAAGTTTCCCATCAGCCGCCCAAACCTAACATT | |
| | | CTCAACTCTGACTGTGAGCAACATGAGCCCTGAAGACAGCAGCA | |
| | | TATATCTCTGCAGCGTCGGTGGACCGCGACAGGGCGAAAAACTG | |
| | | TTTTTTGGCAGTGGAACCCAGCTCTCTGTCTTGG | |
| | Variable-b | SAVISQKPSRDICQRGTSLTIQCQVDSQVTMMFWYRQQPGQSLTLIA | 434 |
| | | TANQGSEATYESGFVIDKFPISRPNLTFSTLTVSNMSPEDSSIYLCSVG | |
| | | GPRQGEKLFFGSGTQLSVL | |
| | CDR1-b | SQVTM | 97 |
| | CDR2-b | ANQGSEA | 98 |
| | CDR3-b | CSVGGPRQGEKLFF | 435 |
| | FWR1-b | SAVISQKPSRDICQRGTSLTIQCQVD | 100 |
| | FWR2-b | MFWYRQQPGQSLTLIAT | 101 |
| | FWR3-b | TYESGFVIDKFPISRPNLTFSTLTVSNMSPEDSSIYL | 102 |
| | FWR4-b | GSGTQLSVL | 45 |
| TCRA- | a-Variable | GATGCTAAGACCACCCAGCCCCCCTCCATGGATTGCGCTGAAGGA | 436 |
| 32 | nucleotide | AGAGCTGCAAACCTGCCTTGTAATCACTCTACCATCAGTGGAAAT | |
| | | GAGTATGTGTATTGGTATCGACAGATTCACTCCCAGGGGCCACAG | |
| | | TATATCATTCATGGTCTAAAAAACAATGAAACCAATGAAATGGCC | |
| | | TCTCTGATCATCACAGAAGACAGAAAGTCCAGCACCTTGATCCTG | |
| | | CCCCACGCTACGCTGAGAGACACTGCTGTGTACTATTGCATCGTC | |
| | | AGATTCTCGTCTGGTTCTGCAAGGCAACTGACCTTTGGATCTGGG | |
| | | ACACAATTGACTGTTTTACCTG | |
| | Variable-a | DAKTTQPPSMDCAEGRAANLPCNHSTISGNEYVYWYRQIHSQGPQY | 437 |
| | | IIHGLKNNETNEMASLIITEDRKSSTLILPHATLRDTAVYYCIVRFSSG | |
| | | SARQLTFGSGTQLTVLP | |
| | CDR1-a | TISGNEY | 88 |
| | CDR2-a | GLKNN | 89 |
| | CDR3-a | CIVRFSSGSARQLTF | 438 |
| | FWR1-a | DAKTTQPPSMDCAEGRAANLPCNHS | 439 |
| | FWR2-a | VYWYRQIHSQGPQYIIH | 92 |
| | FWR3-a | ETNEMASLIITEDRKSSTLILPHATLRDTAVYY | 93 |
| | FWR4-a | GSGTQLTVLP | 213 |
| TCRB- | b-Variable | GATACTGGAGTCTCCCAGAACCCCAGACACAAGATCACAAAGAG | 440 |
| 33 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCTCCCCCGAAACGGCGGGCACTGAAGCTT | |
| | | TCTTTGGACAAGGCACCAGACTCACAGTTGTAG | |
| | Variable-b | DTGVSQNPRHKITKRGQNVTFRCDPISEHNRLYWYRQTLGQGPEFLT | 441 |
| | | YFQNEAQLEKSRLLSDRFSAERPKGSFSTLEIQRTEQGDSAMYLCASS | |
| | | SPETAGTEAFFGQGTRLTVV | |
| | CDR1-b | SEHNR | 442 |
| | CDR2-b | FQNEAQ | 443 |
| | CDR3-b | CASSSPETAGTEAFF | 444 |
| | FWR1-b | DTGVSQNPRHKITKRGQNVTFRCDPI | 445 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GQGTRLTVV | 246 |
| TCRA- | a-Variable | GGACAAAACATTGACCAGCCCACTGAGATGACAGCTACGGAAGG | 448 |
| 33 | nucleotide | TGCCATTGTCCAGATCAACTGCACGTACCAGACATCTGGGTTCAA | |
| | | CGGGCTGTTCTGGTACCAGCAACATGCTGGCGAAGCACCCACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTG | |
| | | AGAGATAGGACGGGAGGAGGAAACAAACTCACCTTTGGGACAGG | |
| | | CACTCAGCTAAAAGTGGAACTCA | |
| | Variable-a | GQNIDQPTEMTATEGAIVQINCTYQTSGFNGLFWYQQHAGEAPTFLS | 449 |
| | | YNVLDGLEEKGRFSSFLSRSKGYSYLLLKELQMKDSASYLCAVRDR | |
| | | TGGGNKLTFGTGTQLKVEL | |
| | CDR1-a | TSGFNG | 450 |
| | CDR2-a | NVLDGL | 451 |
| | CDR3-a | CAVRDRTGGGNKLTF | 452 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GTGTQLKVEL | 456 |
| TCRB- | b-Variable | GACACAGCTGTTTCCCAGACTCCAAAATACCTGGTCACACAGATG | 457 |
| 34.1 | nucleotide | GGAAACGACAAGTCCATTAAATGTGAACAAAATCTGGGCCATGA | |
| | | TACTATGTATTGGTATAAACAGGACTCTAAGAAATTTCTGAAGAT | |
| | | AATGTTTAGCTACAATAATAAGGAGCTCATTATAAATGAAACAGT | |
| | | TCCAAATCGCTTCTCACCTAAATCTCCAGACAAAGCTCACTTAAA | |
| | | TCTTCACATCAATTCCCTGGAGCTTGGTGACTCTGCTGTGTATTTC | |
| | | TGTGCCAGCAGCCACGAGGGAGGCTATGGCTACACCTTCGGTTCG | |
| | | GGGACCAGGTTAACCGTTGTAG | |
| | Variable-b | DTAVSQTPKYLVTQMGNDKSIKCEQNLGHDTMYWYKQDSKKFLKI | 458 |
| | | MFSYNNKELIINETVPNRFSPKSPDKAHLNLHINSLELGDSAVYFCAS | |
| | | SHEGGYGYTFGSGTRLTVV | |
| | CDR1-b | LGHDT | 342 |
| | CDR2-b | YNNKEL | 343 |
| | CDR3-b | CASSHEGGYGYTF | 459 |
| | FWR1-b | DTAVSQTPKYLVTQMGNDKSIKCEQN | 345 |
| | FWR2-b | MYWYKQDSKKFLKIMFS | 346 |
| | FWR3-b | IINETVPNRFSPKSPDKAHLNLHINSLELGDSAVYF | 347 |
| | FWR4-b | GSGTRLTVV | 103 |
| TCRB- | b-Variable | GATGGTGGAATCACTCAGTCCCCAAAGTACCTGTTCAGAAAGGA | 460 |
| 34.2 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGCCCGGGACAGGGTTTCGAGCAGTACTTCGG | |
| | | GCCGGGCACCAGGCTCACGGTCACAG | |
| | Variable-b | DGGITQSPKYLFRKEGQNVTLSCEQNLNHDAMYWYRQDPGQGLRLI | 461 |
| | | YYSQIVNDFQKGDIAEGYSVSREKKESFPLTVTSAQKNPTAFYLCAS | |
| | | SPGQGFEQYFGPGTRLTVT | |
| | CDR1-b | LNHDA | 216 |
| | CDR2-b | SQIVND | 217 |
| | CDR3-b | CASSPGQGFEQYF | 462 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVT | 85 |
| TCRA- | a-Variable | AAACAGGAGGTGACACAGATTCCTGCAGCTCTGAGTGTCCCAGA | 463 |
| 34 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTTCCCGATAACTATGGTCAGAATTTTGTCTTTGGTCCCGGA | |
| | | ACCAGATTGTCCGTGCTGCCCT | |
| | Variable-a | KQEVTQIPAALSVPEGENLVLNCSFTDSAIYNLQWFRQDPGKGLTSL | 464 |
| | | LLIQSSQREQTSGRLNASLDKSSGRSTLYIAASQPGDSATYLCAVPDN | |
| | | YGQNFVFGPGTRLSVLP | |
| | CDR1-a | DSAIYN | 118 |
| | CDR2-a | IQSSQRE | 119 |
| | CDR3-a | CAVPDNYGQNFVF | 465 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GPGTRLSVLP | 272 |
| TCRB- | b-Variable | GATGCTGGAGTTATCCAGTCACCCCGGCACGAGGTGACAGAGAT | 466 |
| 35 | nucleotide | GGGACAAGAAGTGACTCTGAGATGTAAACCAATTTCAGGACACG | |
| | | ACTACCTTTTCTGGTACAGACAGACCATGATGCGGGGACTGGAGT | |
| | | TGCTCATTTACTTTAACAACAACGTTCCGATAGATGATTCAGGGA | |
| | | TGCCCGAGGATCGATTCTCAGCTAAGATGCCTAATGCATCATTCT | |
| | | CCACTCTGAAGATCCAGCCCTCAGAACCCAGGGACTCAGCTGTGT | |
| | | ACTTCTGTGCCAGCAGTTTAAACGGGAGAGAGCTGTTTTTTGGAG | |
| | | AAGGCTCTAGGCTGACCGTACTGG | |
| | Variable-b | DAGVIQSPRHEVTEMGQEVTLRCKPISGHDYLFWYRQTMMRGLELL | 467 |
| | | IYFNNNVPIDDSGMPEDRFSAKMPNASFSTLKIQPSEPRDSAVYFCAS | |
| | | SLNGRELFFGEGSRLTVL | |
| | CDR1-b | SGHDY | 468 |
| | CDR2-b | FNNNVP | 469 |
| | CDR3-b | CASSLNGRELFF | 470 |
| | FWR1-b | DAGVIQSPRHEVTEMGQEVTLRCKPI | 471 |
| | FWR2-b | LFWYRQTMMRGLELLIY | 472 |
| | FWR3-b | IDDSGMPEDRFSAKMPNASFSTLKIQPSEPRDSAVYF | 473 |
| | FWR4-b | GEGSRLTVL | 63 |
| TCRA- | a-Variable | AAACAGGAGGTGACGCAGATTCCTGCAGCTCTGAGTGTCCCAGA | 247 |
| 35 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGAGGCCTCCACGGGAATATGGAAACAAACTGGTCTTTGG | |
| | | CGCAGGAACCATTCTGAGAGTCAAGTCCT | |
| | Variable-a | KQEVTQIPAALSVPEGENLVLNCSFTDSAIYNLQWFRQDPGKGLTSL | 248 |
| | | LLIQSSQREQTSGRLNASLDKSSGRSTLYIAASQPGDSATYLCAVRPP | |
| | | REYGNKLVFGAGTILRVKS | |
| | CDR1-a | DSAIYN | 118 |
| | CDR2-a | IQSSQRE | 119 |
| | CDR3-a | CAVRPPREYGNKLVF | 249 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GAGTILRVKS | 18 |
| TCRB- | b-Variable | GACGCTGGAGTCACCCAAAGTCCCACACACCTGATCAAAACGAG | 474 |
| 36 | nucleotide | AGGACAGCAAGTGACTCTGAGATGCTCTCCTAAGTCTGGGCATGA | |
| | | CACTGTGTCCTGGTACCAACAGGCCCTGGGTCAGGGGCCCCAGTT | |
| | | TATCTTTCAGTATTATGAGGAGGAAGAGAGACAGAGAGGCAACT | |
| | | TCCCTGATCGATTCTCAGGTCACCAGTTCCCTAACTATAGCTCTGA | |
| | | GCTGAATGTGAACGCCTTGTTGCTGGGGGACTCGGCCCTCTATCT | |
| | | CTGTGCCAGCCTCACTAGGGGGATAGTGGGCTACACCTTCGGTTC | |
| | | GGGGACCAGGTTAACCGTTGTAG | |
| | Variable-b | DAGVTQSPTHLIKTRGQQVTLRCSPKSGHDTVSWYQQALGQGPQFIF | 475 |
| | | QYYEEEERQRGNFPDRFSGHQFPNYSSELNVNALLLGDSALYLCASL | |
| | | TRGIVGYTFGSGTRLTVV | |
| | CDR1-b | SGHDT | 331 |
| | CDR2-b | YYEEEE | 332 |
| | CDR3-b | CASLTRGIVGYTF | 476 |
| | FWR1-b | DAGVTQSPTHLIKTRGQQVTLRCSPK | 334 |
| | FWR2-b | VSWYQQALGQGPQFIFQ | 131 |
| | FWR3-b | RQRGNFPDRFSGHQFPNYSSELNVNALLLGDSALYL | 335 |
| | FWR4-b | GSGTRLTVV | 103 |
| TCRA- | a-Variable | GCTCAGTCAGTGGCTCAGCCGGAAGATCAGGTCAACGTTGCTGA | 477 |
| 36 | nucleotide | AGGGAATCCTCTGACTGTGAAATGCACCTATTCAGTCTCTGGAAA | |
| | | CCCTTATCTTTTTTGGTATGTTCAATACCCCAACCGAGGCCTCCAG | |
| | | TTCCTTCTGAAATACATCACAGGGGATAACCTGGTTAAAGGCAGC | |
| | | TATGGCTTTGAAGCTGAATTTAACAAGAGCCAAACCTCCTTCCAC | |
| | | CTGAAGAAACCATCTGCCCTTGTGAGCGACTCCGCTTTGTACTTC | |
| | | TGTGCTGTGAGAGACGGGAGCTATGGTGGTGCTACAAACAAGCT | |
| | | CATCTTTGGAACTGGCACTCTGCTTGCTGTCCAGCCAA | |
| | Variable-a | AQSVAQPEDQVNVAEGNPLTVKCTYSVSGNPYLFWYVQYPNRGLQ | 478 |
| | | FLLKYITGDNLVKGSYGFEAEFNKSQTSFHLKKPSALVSDSALYFCA | |
| | | VRDGSYGGATNKLIFGTGTLLAVQP | |
| | CDR1-a | VSGNPY | 427 |
| | CDR2-a | YITGDNLV | 428 |
| | CDR3-a | CAVRDGSYGGATNKLIF | 479 |
| | FWR1-a | AQSVAQPEDQVNVAEGNPLTVKCTYS | 430 |
| | FWR2-a | LFWYVQYPNRGLQFLLK | 431 |
| | FWR3-a | KGSYGFEAEFNKSQTSFHLKKPSALVSDSALYF | 432 |
| | FWR4-a | GTGTLLAVQP | 480 |
| TCRB- | b-Variable | GAAACGGGAGTTACGCAGACACCAAGACACCTGGTCATGGGAAT | 481 |
| 37 | nucleotide | GACAAATAAGAAGTCTTTGAAATGTGAACAACATCTGGGGCATA | |
| | | ACGCTATGTATTGGTACAAGCAAAGTGCTAAGAAGCCACTGGAG | |
| | | CTCATGTTTGTCTACAACTTTAAAGAACAGACTGAAAACAACAGT | |
| | | GTGCCAAGTCGCTTCTCACCTGAATGCCCCAACAGCTCTCACTTA | |
| | | TTCCTTCACCTACACACCCTGCAGCCAGAAGACTCGGCCCTGTAT | |
| | | CTCTGTGCCAGCAGCCGTTTGGGACTAGCGGGGTTACGGGAGACC | |
| | | CAGTACTTCGGGCCAGGCACGCGGCTCCTGGTGCTCG | |
| | Variable-b | ETGVTQTPRHLVMGMTNKKSLKCEQHLGHNAMYWYKQSAKKPLE | 482 |
| | | LMFVYNFKEQTENNSVPSRFSPECPNSSHLFLHLHTLQPEDSALYLC | |
| | | ASSRLGLAGLRETQYFGPGTRLLVL | |
| | CDR1-b | LGHNA | 169 |
| | CDR2-b | YNFKEQ | 170 |
| | CDR3-b | CASSRLGLAGLRETQYF | 483 |
| | FWR1-b | ETGVTQTPRHLVMGMTNKKSLKCEQH | 172 |
| | FWR2-b | MYWYKQSAKKPLELMFV | 173 |
| | FWR3-b | TENNSVPSRFSPECPNSSHLFLHLHTLQPEDSALYL | 174 |
| | FWR4-b | GPGTRLLVL | 67 |
| TCRA- | a-Variable | GCCCAGACAGTCACTCAGTCTCAACCAGAGATGTCTGTGCAGGA | 484 |
| 37 | nucleotide | GGCAGAGACTGTGACCCTGAGTTGCACATATGACACCAGTGAGA | |
| | | ATAATTATTATTTGTTCTGGTACAAGCAGCCTCCCAGCAGGCAGA | |
| | | TGATTCTCGTTATTCGCCAAGAAGCTTATAAGCAACAGAATGCAA | |
| | | CGGAGAATCGTTTCTCTGTGAACTTCCAGAAAGCAGCCAAATCCT | |
| | | TCAGTCTCAAGATCTCAGACTCACAGCTGGGGGACACTGCGATGT | |
| | | ATTTCTGTGCTTTCATGACCCTCCCTATTACTCGGTCTGGGGCTGG | |
| | | GAGTTACCAACTCACTTTCGGGAAGGGGACCAAACTCTCGGTCAT | |
| | | ACCAA | |
| | Variable-a | AQTVTQSQPEMSVQEAETVTLSCTYDTSENNYYLFWYKQPPSRQMI | 485 |
| | | LVIRQEAYKQQNATENRFSVNFQKAAKSFSLKISDSQLGDTAMYFC | |
| | | AFMTLPITRSGAGSYQLTFGKGTKLSVIP | |
| | CDR1-a | TSENNYY | 486 |
| | CDR2-a | QEAYKQQN | 287 |
| | CDR3-a | CAFMTLPITRSGAGSYQLTF | 487 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDTAMYF | 488 |
| | FWR4-a | GKGTKLSVIP | 489 |
| TCRB- | b-Variable | GAAGCCCAAGTGACCCAGAACCCAAGATACCTCATCACAGTGAC | 490 |
| 38 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGGTTCACAGGGGGATATAACTACGAGCAGTACT | |
| | | TCGGGCCGGGCACCAGGCTCACGGTCACAG | |
| | Variable-b | EAQVTQNPRYLITVTGKKLTVTCSQNMNHEYMSWYRQDPGLGLRQ | 491 |
| | | IYYSMNVEVTDKGDVPEGYKVSRKEKRNFPLILESPSPNQTSLYFCA | |
| | | SRFTGGYNYEQYFGPGTRLTVT | |
| | CDR1-b | MNHEY | 252 |
| | CDR2-b | SMNVEV | 366 |
| | CDR3-b | CASRFTGGYNYEQYF | 492 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GPGTRLTVT | 85 |
| TCRA- | a-Variable | GCCCAGTCTGTGACCCAGCTTGACAGCCAAGTCCCTGTCTTTGAA | 493 |
| 38 | nucleotide | GAAGCCCCTGTGGAGCTGAGGTGCAACTACTCATCGTCTGTTTCA | |
| | | GTGTATCTCTTCTGGTATGTGCAATACCCCAACCAAGGACTCCAG | |
| | | CTTCTCCTGAAGTATTTATCAGGATCCACCCTGGTTAAAGGCATC | |
| | | AACGGTTTTGAGGCTGAATTTAACAAGAGTCAAACTTCCTTCCAC | |
| | | TTGAGGAAACCCTCAGTCCATATAAGCGACACGGCTGAGTACTTC | |
| | | TGTGCTGTGATTGAGGGAGAGGCTGGTGGTACTAGCTATGGAAA | |
| | | GCTGACATTTGGACAAGGGACCATCTTGACTGTCCATCCAA | |
| | Variable-a | AQSVTQLDSQVPVFEEAPVELRCNYSSSVSVYLFWYVQYPNQGLQL | 494 |
| | | LLKYLSGSTLVKGINGFEAEFNKSQTSFHLRKPSVHISDTAEYFCAVI | |
| | | EGEAGGTSYGKLTFGQGTILTVHP | |
| | CDR1-a | SSVSVY | 495 |
| | CDR2-a | YLSGSTLV | 496 |
| | CDR3-a | CAVIEGEAGGTSYGKLTF | 497 |
| | FWR1-a | AQSVTQLDSQVPVFEEAPVELRCNYS | 498 |
| | FWR2-a | LFWYVQYPNQGLQLLLK | 499 |
| | FWR3-a | KGINGFEAEFNKSQTSFHLRKPSVHISDTAEYF | 500 |
| | FWR4-a | GQGTILTVHP | 76 |
| TCRB- | b-Variable | GAAGCCCAAGTGACCCAGAACCCAAGATACCTCATCACAGTGAC | 501 |
| 39 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTCCTCACGCCGGGGAGCTGTTTTTTGGAGAAG | |
| | | GCTCTAGGCTGACCGTACTGG | |
| | Variable-b | EAQVTQNPRYLITVTGKKLTVTCSQNMNHEYMSWYRQDPGLGLRQ | 502 |
| | | IYYSMNVEVTDKGDVPEGYKVSRKEKRNFPLILESPSPNQTSLYFCA | |
| | | SSPHAGELFFGEGSRLTVL | |
| | CDR1-b | MNHEY | 252 |
| | CDR2-b | SMNVEV | 366 |
| | CDR3-b | CASSPHAGELFF | 503 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GEGSRLTVL | 63 |
| TCRA- | a-Variable | GGAATACAAGTGGAGCAGAGTCCTCCAGACCTGATTCTCCAGGA | 504 |
| 39 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTCCCA | |
| | | CCGATGACAACTGACAGCTGGGGGAAATTGCAGTTTGGAGCAGG | |
| | | GACCCAGGTTGTGGTCACCCCAG | |
| | Variable-a | GIQVEQSPPDLILQEGANSTLRCNFSDSVNNLQWFHQNPWGQLINLF | 505 |
| | | YIPSGTKQNGRLSATTVATERYSLLYISSSQTTDSGVYFCAVPPMTTD | |
| | | SWGKLQFGAGTQVVVTP | |
| | CDR1-a | DSVNN | 506 |
| | CDR2-a | IPSGT | 507 |
| | CDR3-a | CAVPPMTTDSWGKLQF | 508 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GAGTQVVVTP | 54 |
| TCRB- | b-Variable | AAGGCTGGAGTCACTCAAACTCCAAGATATCTGATCAAAACGAG | 512 |
| 40 | nucleotide | AGGACAGCAAGTGACACTGAGCTGCTCCCCTATCTCTGGGCATAG | |
| | | GAGTGTATCCTGGTACCAACAGACCCCAGGACAGGGCCTTCAGTT | |
| | | CCTCTTTGAATACTTCAGTGAGACACAGAGAAACAAAGGAAACT | |
| | | TCCCTGGTCGATTCTCAGGGCGCCAGTTCTCTAACTCTCGCTCTGA | |
| | | GATGAATGTGAGCACCTTGGAGCTGGGGGACTCGGCCCTTTATCT | |
| | | TTGCGCCAGCAGCTTACGGCGGGGGCAATCCTACGAGCAGTACTT | |
| | | CGGGCCGGGCACCAGGCTCACGGTCACAG | |
| | Variable-b | KAGVTQTPRYLIKTRGQQVTLSCSPISGHRSVSWYQQTPGQGLQFLF | 513 |
| | | EYFSETQRNKGNFPGRFSGRQFSNSRSEMNVSTLELGDSALYLCASS | |
| | | LRRGQSYEQYFGPGTRLTVT | |
| | CDR1-b | SGHRS | 514 |
| | CDR2-b | YFSETQ | 515 |
| | CDR3-b | CASSLRRGQSYEQYF | 516 |
| | FWR1-b | KAGVTQTPRYLIKTRGQQVTLSCSPI | 517 |
| | FWR2-b | VSWYQQTPGQGLQFLFE | 518 |
| | FWR3-b | RNKGNFPGRFSGRQFSNSRSEMNVSTLELGDSALYL | 519 |
| | FWR4-b | GPGTRLTVT | 85 |
| TCRA- | a-Variable | GATGCTAAGACCACACAGCCAAATTCAATGGAGAGTAACGAAGA | 520 |
| 40 | nucleotide | AGAGCCTGTTCACTTGCCTTGTAACCACTCCACAATCAGTGGAAC | |
| | | TGATTACATACATTGGTATCGACAGCTTCCCTCCCAGGGTCCAGA | |
| | | GTACGTGATTCATGGTCTTACAAGCAATGTGAACAACAGAATGGC | |
| | | CTCTCTGGCAATCGCTGAAGACAGAAAGTCCAGTACCTTGATCCT | |
| | | GCACCGTGCTACCTTGAGAGATGCTGCTGTGTACTACTGCATCCT | |
| | | GGATGCAGGCAACATGCTCACCTTTGGAGGGGGAACAAGGTTAA | |
| | | TGGTCAAACCCC | |
| | Variable-a | DAKTTQPNSMESNEEEPVHLPCNHSTISGTDYIHWYRQLPSQGPEYV | 521 |
| | | IHGLTSNVNNRMASLAIAEDRKSSTLILHRATLRDAAVYYCILDAGN | |
| | | MLTFGGGTRLMVKP | |
| | CDR1-a | TISGTDY | 153 |
| | CDR2-a | GLTSN | 154 |
| | CDR3-a | CILDAGNMLTF | 522 |
| | FWR1-a | DAKTTQPNSMESNEEEPVHLPCNHS | 156 |
| | FWR2-a | IHWYRQLPSQGPEYVIH | 157 |
| | FWR3-a | VNNRMASLAIAEDRKSSTLILHRATLRDAAVYY | 158 |
| | FWR4-a | GGGTRLMVKP | 141 |
| TCRB- | b-Variable | GAACCTGAAGTCACCCAGACTCCCAGCCATCAGGTCACACAGAT | 523 |
| 41 | nucleotide | GGGACAGGAAGTGATCTTGCGCTGTGTCCCCATCTCTAATCACTT | |
| | | ATACTTCTATTGGTACAGACAAATCTTGGGGCAGAAAGTCGAGTT | |
| | | TCTGGTTTCCTTTTATAATAATGAAATCTCAGAGAAGTCTGAAAT | |
| | | ATTCGATGATCAATTCTCAGTTGAAAGGCCTGATGGATCAAATTT | |
| | | CACTCTGAAGATCCGGTCCACAAAGCTGGAGGACTCAGCCATGT | |
| | | ACTTCTGTGCCAGCAAGGAGGGTTGGGGGGACACTGAAGCTTTCT | |
| | | TTGGACAAGGCACCAGACTCACAGTTGTAG | |
| | Variable-b | EPEVTQTPSHQVTQMGQEVILRCVPISNHLYFYWYRQILGQKVEFLV | 524 |
| | | SFYNNEISEKSEIFDDQFSVERPDGSNFTLKIRSTKLEDSAMYFCASKE | |
| | | GWGDTEAFFGQGTRLTVV | |
| | CDR1-b | SNHLY | 525 |
| | CDR2-b | FYNNEI | 526 |
| | CDR3-b | CASKEGWGDTEAFF | 527 |
| | FWR1-b | EPEVTQTPSHQVTQMGQEVILRCVPI | 528 |
| | FWR2-b | FYWYRQILGQKVEFLVS | 529 |
| | FWR3-b | SEKSEIFDDQFSVERPDGSNFTLKIRSTKLEDSAMYF | 530 |
| | FWR4-b | GQGTRLTVV | 246 |
| TCRA- | a-Variable | ACCCAGCTGCTGGAGCAGAGCCCTCAGTTTCTAAGCATCCAAGAG | 531 |
| 41 | nucleotide | GGAGAAAATCTCACTGTGTACTGCAACTCCTCAAGTGTTTTTTCC | |
| | | AGCTTACAATGGTACAGACAGGAGCCTGGGGAAGGTCCTGTCCT | |
| | | CCTGGTGACAGTAGTTACGGGTGGAGAAGTGAAGAAGCTGAAGA | |
| | | GACTAACCTTTCAGTTTGGTGATGCAAGAAAGGACAGTTCTCTCC | |
| | | ACATCACTGCGGCCCAGCCTGGTGATACAGGCCTCTACCTCTGTG | |
| | | CAGGAGTAGGAATGGATAGCAGCTATAAATTGATCTTCGGGAGT | |
| | | GGGACCAGACTGCTGGTCAGGCCTG | |
| | Variable-a | TQLLEQSPQFLSIQEGENLTVYCNSSSVFSSLQWYRQEPGEGPVLLVT | 532 |
| | | VVTGGEVKKLKRLTFQFGDARKDSSLHITAAQPGDTGLYLCAGVG | |
| | | MDSSYKLIFGSGTRLLVRP | |
| | CDR1-a | SVFSS | 533 |
| | CDR2-a | VVTGGEV | 534 |
| | CDR3-a | CAGVGMDSSYKLIF | 535 |
| | FWR1-a | TQLLEQSPQFLSIQEGENLTVYCNSS | 536 |
| | FWR2-a | LQWYRQEPGEGPVLLVT | 537 |
| | FWR3-a | KKLKRLTFQFGDARKDSSLHITAAQPGDTGLYL | 538 |
| | FWR4-a | GSGTRLLVRP | 539 |
| TCRB- | b-Variable | AATGCTGGTGTCACTCAGACCCCAAAATTCCAGGTCCTGAAGACA | 540 |
| 42 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTTCCCGACAGGGTTATCAGCCCCAGCATTTTGG | |
| | | TGATGGGACTCGACTCTCCATCCTAG | |
| | Variable-b | NAGVTQTPKFQVLKTGQSMTLQCAQDMNHEYMSWYRQDPGMGLR | 541 |
| | | LIHYSVGAGITDQGEVPNGYNVSRSTTEDFPLRLLSAAPSQTSVYFCA | |
| | | SSSRQGYQPQHFGDGTRLSIL | |
| | CDR1-b | MNHEY | 252 |
| | CDR2-b | SVGAGI | 253 |
| | CDR3-b | CASSSRQGYQPQHF | 542 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GDGTRLSIL | 27 |
| TCRA- | a-Variable | GCTCAGACAGTCACTCAGTCTCAACCAGAGATGTCTGTGCAGGAG | 543 |
| 42 | nucleotide | GCAGAGACCGTGACCCTGAGCTGCACATATGACACCAGTGAGAG | |
| | | TGATTATTATTTATTCTGGTACAAGCAGCCTCCCAGCAGGCAGAT | |
| | | GATTCTCGTTATTCGCCAAGAAGCTTATAAGCAACAGAATGCAAC | |
| | | AGAGAATCGTTTCTCTGTGAACTTCCAGAAAGCAGCCAAATCCTT | |
| | | CAGTCTCAAGATCTCAGACTCACAGCTGGGGGATGCCGCGATGTA | |
| | | TTTCTGTGCTTATAGGAGCGGAAACACACCTCTTGTCTTTGGAAA | |
| | | GGGCACAAGACTTTCTGTGATTGCAA | |
| | Variable-a | AQTVTQSQPEMSVQEAETVTLSCTYDTSESDYYLFWYKQPPSRQMI | 544 |
| | | LVIRQEAYKQQNATENRFSVNFQKAAKSFSLKISDSQLGDAAMYFC | |
| | | AYRSGNTPLVFGKGTRLSVIA | |
| | CDR1-a | TSESDYY | 286 |
| | CDR2-a | QEAYKQQN | 287 |
| | CDR3-a | CAYRSGNTPLVF | 545 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDAAMYF | 291 |
| | FWR4-a | GKGTRLSVIA | 546 |
| TCRB- | b-Variable | AATGCTGGTGTCACTCAGACCCCAAAATTCCAGGTCCTGAAGACA | 547 |
| 43 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTTACTCGCTGCTGTCGCCCTCCTACGAGCAGTA | |
| | | CTTCGGGCCGGGCACCAGGCTCACGGTCACAG | |
| | Variable-b | NAGVTQTPKFQVLKTGQSMTLQCAQDMNHEYMSWYRQDPGMGLR | 548 |
| | | LIHYSVGAGITDQGEVPNGYNVSRSTTEDFPLRLLSAAPSQTSVYFCA | |
| | | SSYSLLSPSYEQYFGPGTRLTVT | |
| | CDR1-b | MNHEY | 252 |
| | CDR2-b | SVGAGI | 253 |
| | CDR3-b | CASSYSLLSPSYEQYF | 549 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GPGTRLTVT | 85 |
| TCRA- | a-Variable | AGTCAACAGGGAGAAGAGGATCCTCAGGCCTTGAGCATCCAGGA | 550 |
| 43 | nucleotide | GGGTGAAAATGCCACCATGAACTGCAGTTACAAAACTAGTATAA | |
| | | ACAATTTACAGTGGTATAGACAAAATTCAGGTAGAGGCCTTGTCC | |
| | | ACCTAATTTTAATACGTTCAAATGAAAGAGAGAAACACAGTGGA | |
| | | AGATTAAGAGTCACGCTTGACACTTCCAAGAAAAGCAGTTCCTTG | |
| | | TTGATCACGGCTTCCCGGGCAGCAGACACTGCTTCTTACTTCTGT | |
| | | GCTACGGACCACCGAACTAGCTATGGAAAGCTGACATTTGGACA | |
| | | AGGGACCATCTTGACTGTCCATCCAA | |
| | Variable-a | SQQGEEDPQALSIQEGENATMNCSYKTSINNLQWYRQNSGRGLVHL | 551 |
| | | ILIRSNEREKHSGRLRVTLDTSKKSSSLLITASRAADTASYFCATDHR | |
| | | TSYGKLTFGQGTILTVHP | |
| | CDR1-a | TSINN | 552 |
| | CDR2-a | IRSNERE | 553 |
| | CDR3-a | CATDHRTSYGKLTF | 554 |
| | FWR1-a | SQQGEEDPQALSIQEGENATMNCSYK | 555 |
| | FWR2-a | LQWYRQNSGRGLVHLIL | 556 |
| | FWR3-a | KHSGRLRVTLDTSKKSSSLLITASRAADTASYF | 557 |
| | FWR4-a | GQGTILTVHP | 76 |
| TCRB- | b-Variable | GAGACTGGAGTCACCCAAAGTCCCACACACCTGATCAAAACGAG | 558 |
| 44 | nucleotide | AGGACAGCAAGTGACTCTGAGATGCTCTTCTCAGTCTGGGCACAA | |
| | | CACTGTGTCCTGGTACCAACAGGCCCTGGGTCAGGGGCCCCAGTT | |
| | | TATCTTTCAGTATTATAGGGAGGAAGAGAATGGCAGAGGAAACT | |
| | | TCCCTCCTAGATTCTCAGGTCTCCAGTTCCCTAATTATAGCTCTGA | |
| | | GCTGAATGTGAACGCCTTGGAGCTGGACGACTCGGCCCTGTATCT | |
| | | CTGTGCCAGCAGCTCAGAACGGGGACATAAGGGCTACACCTTCG | |
| | | GTTCGGGGACCAGGTTAACCGTTGTAG | |
| | Variable-b | ETGVTQSPTHLIKTRGQQVTLRCSSQSGHNTVSWYQQALGQGPQFIF | 559 |
| | | QYYREEENGRGNFPPRFSGLQFPNYSSELNVNALELDDSALYLCASS | |
| | | SERGHKGYTFGSGTRLTVV | |
| | CDR1-b | SGHNT | 127 |
| | CDR2-b | YYREEE | 128 |
| | CDR3-b | CASSSERGHKGYTF | 560 |
| | FWR1-b | ETGVTQSPTHLIKTRGQQVTLRCSSQ | 130 |
| | FWR2-b | VSWYQQALGQGPQFIFQ | 131 |
| | FWR3-b | NGRGNFPPRFSGLQFPNYSSELNVNALELDDSALYL | 132 |
| | FWR4-b | GSGTRLTVV | 103 |
| TCRA- | a-Variable | AAAAACCAAGTGGAGCAGAGTCCTCAGTCCCTGATCATCCTGGA | 561 |
| 44 | nucleotide | GGGAAAGAACTGCACTCTTCAATGCAATTATACAGTGAGCCCCTT | |
| | | CAGCAACTTAAGGTGGTATAAGCAAGATACTGGGAGAGGTCCTG | |
| | | TTTCCCTGACAATCATGACTTTCAGTGAGAACACAAAGTCGAACG | |
| | | GAAGATATACAGCAACTCTGGATGCAGACACAAAGCAAAGCTCT | |
| | | CTGCACATCACAGCCTCCCAGCTCAGCGATTCAGCCTCCTACATC | |
| | | TGTGTGTTATCCGGTGGTGCTACAAACAAGCTCATCTTTGGAACT | |
| | | GGCACTCTGCTTGCTGTCCAGCCAA | |
| | Variable-a | KNQVEQSPQSLIILEGKNCTLQCNYTVSPFSNLRWYKQDTGRGPVSL | 562 |
| | | TIMTFSENTKSNGRYTATLDADTKQSSLHITASQLSDSASYICVLSGG | |
| | | ATNKLIFGTGTLLAVQP | |
| | CDR1-a | VSPFSN | 135 |
| | CDR2-a | MTFSENT | 136 |
| | CDR3-a | CVLSGGATNKLIF | 563 |
| | FWR1-a | KNQVEQSPQSLIILEGKNCTLQCNYT | 138 |
| | FWR2-a | LRWYKQDTGRGPVSLTI | 139 |
| | FWR3-a | KSNGRYTATLDADTKQSSLHITASQLSDSASYI | 140 |
| | FWR4-a | GTGTLLAVQP | 480 |
| TCRB- | b-Variable | GATGGTGGAATCACTCAGTCCCCAAAGTACCTGTTCAGAAAGGA | 564 |
| 45 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGTACGACTAGCGGGAGTTTGGAGACCCAGTA | |
| | | CTTCGGGCCAGGCACGCGGCTCCTGGTGCTCG | |
| | Variable-b | DGGITQSPKYLFRKEGQNVTLSCEQNLNHDAMYWYRQDPGQGLRLI | 565 |
| | | YYSQIVNDFQKGDIAEGYSVSREKKESFPLTVTSAQKNPTAFYLCAS | |
| | | STTSGSLETQYFGPGTRLLVL | |
| | CDR1-b | LNHDA | 216 |
| | CDR2-b | SQIVND | 217 |
| | CDR3-b | CASSTTSGSLETQYF | 566 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLLVL | 67 |
| TCRA- | a-Variable | AAAAACCAAGTGGAGCAGAGTCCTCAGTCCCTGATCATCCTGGA | 567 |
| 45 | nucleotide | GGGAAAGAACTGCACTCTTCAATGCAATTATACAGTGAGCCCCTT | |
| | | CAGCAACTTAAGGTGGTATAAGCAAGATACTGGGAGAGGTCCTG | |
| | | TTTCCCTGACAATCATGACTTTCAGTGAGAACACAAAGTCGAACG | |
| | | GAAGATATACAGCAACTCTGGATGCAGACACAAAGCAAAGCTCT | |
| | | CTGCACATCACAGCCTCCCAGCTCAGCGATTCAGCCTCCTACATC | |
| | | TGTGTGGTGAGCGCGGGCAGGGAATATGGAAACAAGCTGGTCTT | |
| | | TGGCGCAGGAACCATTCTGAGAGTCAAGTCCT | |
| | Variable-a | KNQVEQSPQSLIILEGKNCTLQCNYTVSPFSNLRWYKQDTGRGPVSL | 568 |
| | | TIMTFSENTKSNGRYTATLDADTKQSSLHITASQLSDSASYICVVSAG | |
| | | REYGNKLVFGAGTILRVKS | |
| | CDR1-a | VSPFSN | 135 |
| | CDR2-a | MTFSENT | 136 |
| | CDR3-a | CVVSAGREYGNKLVF | 569 |
| | FWR1-a | KNQVEQSPQSLIILEGKNCTLQCNYT | 138 |
| | FWR2-a | LRWYKQDTGRGPVSLTI | 139 |
| | FWR3-a | KSNGRYTATLDADTKQSSLHITASQLSDSASYI | 140 |
| | FWR4-a | GAGTILRVKS | 18 |
| TCRB- | b-Variable | GAAGCCCAAGTGACCCAGAACCCAAGATACCTCATCACAGTGAC | 570 |
| 46 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTTCGACAGGCATGAACACTGAAGCTTTCTTTG | |
| | | GACAAGGCACCAGACTCACAGTTGTAG | |
| | Variable-b | EAQVTQNPRYLITVTGKKLTVTCSQNMNHEYMSWYRQDPGLGLRQ | 571 |
| | | IYYSMNVEVTDKGDVPEGYKVSRKEKRNFPLILESPSPNQTSLYFCA | |
| | | SSSTGMNTEAFFGQGTRLTVV | |
| | CDR1-b | MNHEY | 252 |
| | CDR2-b | SMNVEV | 366 |
| | CDR3-b | CASSSTGMNTEAFF | 572 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GQGTRLTVV | 246 |
| TCRA- | a-Variable | GGACAACAGGTAATGCAAATTCCTCAGTACCAGCATGTACAAGA | 573 |
| 46 | nucleotide | AGGAGAAGACTTCACCACGTACTGCAATTCCTCAACTACTTTAAG | |
| | | CAATATACAGTGGTATAAGCAAAGGCCTGGTGGACATCCCGTTTT | |
| | | TTTGATACAGTTAGTGAAGAGTGGAGAAGTGAAGAAGCAGAAAA | |
| | | GACTGACATTTCAGTTTGGAGAAGCAAAAAAGAACAGCTCCCTG | |
| | | CACATCACAGCCACCCAGACTACAGATGTAGGAACCTACTTCTGT | |
| | | GCAGGGCTAACTCGGGATAGCAACTATCAGTTAATCTGGGGCGCT | |
| | | GGGACCAAGCTAATTATAAAGCCAG | |
| | Variable-a | GQQVMQIPQYQHVQEGEDFTTYCNSSTTLSNIQWYKQRPGGHPVFLI | 574 |
| | | QLVKSGEVKKQKRLTFQFGEAKKNSSLHITATQTTDVGTYFCAGLT | |
| | | RDSNYQLIWGAGTKLIIKP | |
| | CDR1-a | TTLSN | 575 |
| | CDR2-a | LVKSGEV | 576 |
| | CDR3-a | CAGLTRDSNYQLIW | 577 |
| | FWR1-a | GQQVMQIPQYQHVQEGEDFTTYCNSS | 578 |
| | FWR2-a | IQWYKQRPGGHPVFLIQ | 579 |
| | FWR3-a | KKQKRLTFQFGEAKKNSSLHITATQTTDVGTYF | 580 |
| | FWR4-a | GAGTKLIIKP | 356 |
| TCRB- | b-Variable | GATGGTGGAATCACTCAGTCCCCAAAGTACCTGTTCAGAAAGGA | 581 |
| 47 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGTTCGTCCCCCTTTGGCTACACCTTCGGTTCG | |
| | | GGGACCAGGTTAACCGTTGTAG | |
| | Variable-b | DGGITQSPKYLFRKEGQNVTLSCEQNLNHDAMYWYRQDPGQGLRLI | 582 |
| | | YYSQIVNDFQKGDIAEGYSVSREKKESFPLTVTSAQKNPTAFYLCAS | |
| | | SSSPFGYTFGSGTRLTVV | |
| | CDR1-b | LNHDA | 216 |
| | CDR2-b | SQIVND | 217 |
| | CDR3-b | CASSSSPFGYTF | 583 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GSGTRLTVV | 103 |
| TCRA- | a-Variable | AAACAGGAGGTGACGCAGATTCCTGCAGCTCTGAGTGTCCCAGA | 584 |
| 47 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGAGCGATACCTCAGGAACCTACAAATACATCTTTGGAAC | |
| | | AGGCACCAGGCTGAAGGTTTTAGCAA | |
| | Variable-a | KQEVTQIPAALSVPEGENLVLNCSFTDSAIYNLQWFRQDPGKGLTSL | 585 |
| | | LLIQSSQREQTSGRLNASLDKSSGRSTLYIAASQPGDSATYLCAVSDT | |
| | | SGTYKYIFGTGTRLKVLA | |
| | CDR1-a | DSAIYN | 118 |
| | CDR2-a | IQSSQRE | 119 |
| | CDR3-a | CAVSDTSGTYKYIF | 586 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GTGTRLKVLA | 587 |
| TCRB- | b-Variable | GATACTGGAGTCTCCCAGGACCCCAGACACAAGATCACAAAGAG | 588 |
| 48 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCCACGTCGATGGGGGCGGTGAGACCCAG | |
| | | TACTTCGGGCCAGGCACGCGGCTCCTGGTGCTCG | |
| | Variable-b | DTGVSQDPRHKITKRGQNVTFRCDPISEHNRLYWYRQTLGQGPEFLT | 589 |
| | | YFQNEAQLEKSRLLSDRFSAERPKGSFSTLEIQRTEQGDSAMYLCASS | |
| | | HVDGGGETQYFGPGTRLLVL | |
| | CDR1-b | SEHNR | 442 |
| | CDR2-b | FQNEAQ | 443 |
| | CDR3-b | CASSHVDGGGETQYF | 590 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GPGTRLLVL | 67 |
| TCRA- | a-Variable | CTTGCTAAGACCACCCAGCCCATCTCCATGGACTCATATGAAGGA | 592 |
| 48 | nucleotide | CAAGAAGTGAACATAACCTGTAGCCACAACAACATTGCTACAAA | |
| | | TGATTATATCACGTGGTACCAACAGTTTCCCAGCCAAGGACCACG | |
| | | ATTTATTATTCAAGGATACAAGACAAAAGTTACAAACGAAGTGG | |
| | | CCTCCCTGTTTATCCCTGCCGACAGAAAGTCCAGCACTCTGAGCC | |
| | | TGCCCCGGGTTTCCCTGAGCGACACTGCTGTGTACTACTGCCTCG | |
| | | CTACAGGAGGAAGCTACATACCTACATTTGGAAGAGGAACCAGC | |
| | | CTTATTGTTCATCCGT | |
| | Variable-a | LAKTTQPISMDSYEGQEVNITCSHNNIATNDYITWYQQFPSQGPRFII | 593 |
| | | QGYKTKVTNEVASLFIPADRKSSTLSLPRVSLSDTAVYYCLATGGSYI | |
| | | PTFGRGTSLIVHP | |
| | CDR1-a | NIATNDY | 594 |
| | CDR2-a | GYKTK | 595 |
| | CDR3-a | CLATGGSYIPTF | 596 |
| | FWR1-a | LAKTTQPISMDSYEGQEVNITCSHN | 597 |
| | FWR2-a | ITWYQQFPSQGPRFIIQ | 598 |
| | FWR3-a | VTNEVASLFIPADRKSSTLSLPRVSLSDTAVYY | 599 |
| | FWR4-a | GRGTSLIVHP | 600 |
| TCRB- | b-Variable | AGTGCTGTCATCTCTCAAAAGCCAAGCAGGGATATCTGTCAACGT | 601 |
| 49 | nucleotide | GGAACCTCCCTGACGATCCAGTGTCAAGTCGATAGCCAAGTCACC | |
| | | ATGATGTTCTGGTACCGTCAGCAACCTGGACAGAGCCTGACACTG | |
| | | ATCGCAACTGCAAATCAGGGCTCTGAGGCCACATATGAGAGTGG | |
| | | ATTTGTCATTGACAAGTTTCCCATCAGCCGCCCAAACCTAACATT | |
| | | CTCAACTCTGACTGTGAGCAACATGAGCCCTGAAGACAGCAGCA | |
| | | TATATCTCTGCAGCGTTGAAATGGGGCGGAGCTCCTACAATGAGC | |
| | | AGTTCTTCGGGCCAGGGACACGGCTCACCGTGCTAG | |
| | Variable-b | SAVISQKPSRDICQRGTSLTIQCQVDSQVTMMFWYRQQPGQSLTLIA | 602 |
| | | TANQGSEATYESGFVIDKFPISRPNLTFSTLTVSNMSPEDSSIYLCSVE | |
| | | MGRSSYNEQFFGPGTRLTVL | |
| | CDR1-b | SQVTM | 97 |
| | CDR2-b | ANQGSEA | 98 |
| | CDR3-b | CSVEMGRSSYNEQFF | 603 |
| | FWR1-b | SAVISQKPSRDICQRGTSLTIQCQVD | 100 |
| | FWR2-b | MFWYRQQPGQSLTLIAT | 101 |
| | FWR3-b | TYESGFVIDKFPISRPNLTFSTLTVSNMSPEDSSIYL | 102 |
| | FWR4-b | GPGTRLTVL | 9 |
| TCRA- | a-Variable | GGACAACAGGTAATGCAAATTCCTCAGTACCAGCATGTACAAGA | 604 |
| 49 | nucleotide | AGGAGAAGACTTCACCACGTACTGCAATTCCTCAACTACTTTAAG | |
| | | CAATATACAGTGGTATAAGCAAAGGCCTGGTGGACATCCCGTTTT | |
| | | TTTGATACAGTTAGTGAAGAGTGGAGAAGTGAAGAAGCAGAAAA | |
| | | GACTGACATTTCAGTTTGGAGAAGCAAAAAAGAACAGCTCCCTG | |
| | | CACATCACAGCCACCCAGACTACAGATGTAGGAACCTACTTCTGT | |
| | | GCAGGGACGTATGACAAGATCATCTTTGGAAAAGGGACACGACT | |
| | | TCATATTCTCCCCA | |
| | Variable-a | GQQVMQIPQYQHVQEGEDFTTYCNSSTTLSNIQWYKQRPGGHPVFLI | 605 |
| | | QLVKSGEVKKQKRLTFQFGEAKKNSSLHITATQTTDVGTYFCAGTY | |
| | | DKIIFGKGTRLHILP | |
| | CDR1-a | TTLSN | 575 |
| | CDR2-a | LVKSGEV | 576 |
| | CDR3-a | CAGTYDKIIF | 606 |
| | FWR1-a | GQQVMQIPQYQHVQEGEDFTTYCNSS | 578 |
| | FWR2-a | IQWYKQRPGGHPVFLIQ | 579 |
| | FWR3-a | KKQKRLTFQFGEAKKNSSLHITATQTTDVGTYF | 580 |
| | FWR4-a | GKGTRLHILP | 607 |
| TCRB- | b-Variable | GAAGCCCAAGTGACCCAGAACCCAAGATACCTCATCACAGTGAC | 608 |
| 50 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTTTATCAACCCCCTACGAGCAGTACTTCGGGC | |
| | | CGGGCACCAGGCTCACGGTCACAG | |
| | Variable-b | EAQVTQNPRYLITVTGKKLTVTCSQNMNHEYMSWYRQDPGLGLRQ | 609 |
| | | IYYSMNVEVTDKGDVPEGYKVSRKEKRNFPLILESPSPNQTSLYFCA | |
| | | SSLSTPYEQYFGPGTRLTVT | |
| | CDR1-b | MNHEY | 252 |
| | CDR2-b | SMNVEV | 366 |
| | CDR3-b | CASSLSTPYEQYF | 610 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GPGTRLTVT | 85 |
| TCRA- | a-Variable | GACCAGCAAGTTAAGCAAAATTCACCATCCCTGAGCGTCCAGGA | 611 |
| 50 | nucleotide | AGGAAGAATTTCTATTCTGAACTGTGACTATACTAACAGCATGTT | |
| | | TGATTATTTCCTATGGTACAAAAAATACCCTGCTGAAGGTCCTAC | |
| | | ATTCCTGATATCTATAAGTTCCATTAAGGATAAAAATGAAGATGG | |
| | | AAGATTCACTGTTTTCTTAAACAAAAGTGCCAAGCACCTCTCTCT | |
| | | GCACATTGTGCCCTCCCAGCCTGGAGACTCTGCAGTGTACTTCTG | |
| | | TGCAGCAAGCGGGGTGGCAGGTGAATATGGAAACAAGCTGGTCT | |
| | | TTGGCGCAGGAACCATTCTGAGAGTCAAGTCCT | |
| | Variable-a | DQQVKQNSPSLSVQEGRISILNCDYTNSMFDYFLWYKKYPAEGPTFL | 612 |
| | | ISISSIKDKNEDGRFTVFLNKSAKHLSLHIVPSQPGDSAVYFCAASGV | |
| | | AGEYGNKLVFGAGTILRVKS | |
| | CDR1-a | NSMFDY | 303 |
| | CDR2-a | ISSIKDK | 304 |
| | CDR3-a | CAASGVAGEYGNKLVF | 613 |
| | FWR1-a | DQQVKQNSPSLSVQEGRISILNCDYT | 306 |
| | FWR2-a | FLWYKKYPAEGPTFLIS | 307 |
| | FWR3-a | NEDGRFTVFLNKSAKHLSLHIVPSQPGDSAVYF | 308 |
| | FWR4-a | GAGTILRVKS | 18 |
| TCRB- | b-Variable | ACTGCTGGGATCACCCAGGCACCAACATCTCAGATCCTGGCAGCA | 614 |
| 51 | nucleotide | GGACGGAGCATGACACTGAGATGTACCCAGGATATGAGACATAA | |
| | | TGCCATGTACTGGTATAGACAAGATCTAGGACTGGGGCTAAGGCT | |
| | | CATCCATTATTCAAATACTGCAGGTACCACTGGCAAAGGAGAAGT | |
| | | CCCTGATGGTTATAGTGTCTCCAGAGCAAACACAGATGATTTCCC | |
| | | CCTCACGTTGGCGTCTGCTGTACCCTCTCAGACATCTGTGTACTTC | |
| | | TGTGCCAGCAGCGCGGGACAGGGGGCCGGCGATGAGCAGTTCTT | |
| | | CGGGCCAGGGACACGGCTCACCGTGCTAG | |
| | Variable-b | TAGITQAPTSQILAAGRSMTLRCTQDMRHNAMYWYRQDLGLGLRLI | 615 |
| | | HYSNTAGTTGKGEVPDGYSVSRANTDDFPLTLASAVPSQTSVYFCAS | |
| | | SAGQGAGDEQFFGPGTRLTVL | |
| | CDR1-b | MRHNA | 295 |
| | CDR2-b | SNTAGT | 296 |
| | CDR3-b | CASSAGQGAGDEQFF | 616 |
| | FWR1-b | TAGITQAPTSQILAAGRSMTLRCTQD | 298 |
| | FWR2-b | MYWYRQDLGLGLRLIHY | 299 |
| | FWR3-b | TGKGEVPDGYSVSRANTDDFPLTLASAVPSQTSVYF | 300 |
| | FWR4-b | GPGTRLTVL | 9 |
| TCRA- | a-Variable | GGACAAAACATTGACCAGCCCACTGAGATGACAGCTACGGAAGG | 617 |
| 51 | nucleotide | TGCCATTGTCCAGATCAACTGCACGTACCAGACATCTGGGTTCAA | |
| | | CGGGCTGTTCTGGTACCAGCAACATGCTGGCGAAGCACCCACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTG | |
| | | AGATCCCCGCATGGCTCTGGGGCTGGGAGTTACCAACTCACTTTC | |
| | | GGGAAGGGGACCAAACTCTCGGTCATACCAA | |
| | Variable-a | GQNIDQPTEMTATEGAIVQINCTYQTSGFNGLFWYQQHAGEAPTFLS | 618 |
| | | YNVLDGLEEKGRFSSFLSRSKGYSYLLLKELQMKDSASYLCAVRSPH | |
| | | GSGAGSYQLTFGKGTKLSVIP | |
| | CDR1-a | TSGFNG | 450 |
| | CDR2-a | NVLDGL | 451 |
| | CDR3-a | CAVRSPHGSGAGSYQLTF | 619 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GKGTKLSVIP | 489 |
| TCRB- | b-Variable | GATACTGGAGTCTCCCAGGACCCCAGACACAAGATCACAAAGAG | 620 |
| 52 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCGACGACGGTAGCTCCTACGAGCAGTACT | |
| | | TCGGGCCGGGCACCAGGCTCACGGTCACAG | |
| | Variable-b | DTGVSQDPRHKITKRGQNVTFRCDPISEHNRLYWYRQTLGQGPEFLT | 621 |
| | | YFQNEAQLEKSRLLSDRFSAERPKGSFSTLEIQRTEQGDSAMYLCASS | |
| | | DDGSSYEQYFGPGTRLTVT | |
| | CDR1-b | SEHNR | 442 |
| | CDR2-b | FQNEAQ | 443 |
| | CDR3-b | CASSDDGSSYEQYF | 622 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GPGTRLTVT | 85 |
| TCRA- | a-Variable | GACCAGCAAGTTAAGCAAAATTCACCATCCCTGAGCGTCCAGGA | 623 |
| 52 | nucleotide | AGGAAGAATTTCTATTCTGAACTGTGACTATACTAACAGCATGTT | |
| | | TGATTATTTCCTATGGTACAAAAAATACCCTGCTGAAGGTCCTAC | |
| | | ATTCCTGATATCTATAAGTTCCATTAAGGATAAAAATGAAGATGG | |
| | | AAGATTCACTGTTTTCTTAAACAAAAGTGCCAAGCACCTCTCTCT | |
| | | GCACATTGTGCCCTCCCAGCCTGGAGACTCTGCAGTGTACTTCTG | |
| | | TGCAGCAAGCGGAGGGGTAGGCACCAATGCAGGCAAATCAACCT | |
| | | TTGGGGATGGGACTACGCTCACTGTGAAGCCAA | |
| | Variable-a | DQQVKQNSPSLSVQEGRISILNCDYTNSMFDYFLWYKKYPAEGPTFL | 624 |
| | | ISISSIKDKNEDGRFTVFLNKSAKHLSLHIVPSQPGDSAVYFCAASGG | |
| | | VGTNAGKSTFGDGTTLTVKP | |
| | CDR1-a | NSMFDY | 303 |
| | CDR2-a | ISSIKDK | 304 |
| | CDR3-a | CAASGGVGTNAGKSTF | 625 |
| | FWR1-a | DQQVKQNSPSLSVQEGRISILNCDYT | 306 |
| | FWR2-a | FLWYKKYPAEGPTFLIS | 307 |
| | FWR3-a | NEDGRFTVFLNKSAKHLSLHIVPSQPGDSAVYF | 308 |
| | FWR4-a | GDGTTLTVKP | 626 |
| TCRB- | b-Variable | GGAGCTGGAGTCTCCCAGTCCCCCAGTAACAAGGTCACAGAGAA | 627 |
| 53 | nucleotide | GGGAAAGGATGTAGAGCTCAGGTGTGATCCAATTTCAGGTCATA | |
| | | CTGCCCTTTACTGGTACCGACAGAGCCTGGGGCAGGGCCTGGAGT | |
| | | TTTTAATTTACTTCCAAGGCAACAGTGCACCAGACAAATCAGGGC | |
| | | TGCCCAGTGATCGCTTCTCTGCAGAGAGGACTGGGGGATCCGTCT | |
| | | CCACTCTGACGATCCAGCGCACACAGCAGGAGGACTCGGCCGTG | |
| | | TATCTCTGTGCCAGCAGCTTCGGGGGGGACCCACGCACTCAGCCC | |
| | | CAGCATTTTGGTGATGGGACTCGACTCTCCATCCTAG | |
| | Variable-b | GAGVSQSPSNKVTEKGKDVELRCDPISGHTALYWYRQSLGQGLEFLI | 628 |
| | | YFQGNSAPDKSGLPSDRFSAERTGGSVSTLTIQRTQQEDSAVYLCAS | |
| | | SFGGDPRTQPQHFGDGTRLSIL | |
| | CDR1-b | SGHTA | 263 |
| | CDR2-b | FQGNSA | 629 |
| | CDR3-b | CASSFGGDPRTQPQHF | 630 |
| | FWR1-b | GAGVSQSPSNKVTEKGKDVELRCDPI | 631 |
| | FWR2-b | LYWYRQSLGQGLEFLIY | 632 |
| | FWR3-b | PDKSGLPSDRFSAERTGGSVSTLTIQRTQQEDSAVYL | 633 |
| | FWR4-b | GDGTRLSIL | 27 |
| TCRA- | a-Variable | CTTGCTAAGACCACCCAGCCCATCTCCATGGACTCATATGAAGGA | 634 |
| 53 | nucleotide | CAAGAAGTGAACATAACCTGTAGCCACAACAACATTGCTACAAA | |
| | | TGATTATATCACGTGGTACCAACAGTTTCCCAGCCAAGGACCACG | |
| | | ATTTATTATTCAAGGATACAAGACAAAAGTTACAAACGAAGTGG | |
| | | CCTCCCTGTTTATCCCTGCCGACAGAAAGTCCAGCACTCTGAGCC | |
| | | TGCCCCGGGTTTCCCTGAGCGACACTGCTGTGTACTACTGCCTCG | |
| | | TGGGTCTATCTAACTTTGGAAATGAGAAATTAACCTTTGGGACTG | |
| | | GAACAAGACTCACCATCATACCCA | |
| | Variable-a | LAKTTQPISMDSYEGQEVNITCSHNNIATNDYITWYQQFPSQGPRFII | 635 |
| | | QGYKTKVTNEVASLFIPADRKSSTLSLPRVSLSDTAVYYCLVGLSNF | |
| | | GNEKLTFGTGTRLTIIP | |
| | CDR1-a | NIATNDY | 594 |
| | CDR2-a | GYKTK | 595 |
| | CDR3-a | CLVGLSNFGNEKLTF | 636 |
| | FWR1-a | LAKTTQPISMDSYEGQEVNITCSHN | 597 |
| | FWR2-a | ITWYQQFPSQGPRFIIQ | 598 |
| | FWR3-a | VTNEVASLFIPADRKSSTLSLPRVSLSDTAVYY | 599 |
| | FWR4-a | GTGTRLTIIP | 292 |
| TCRB- | b-Variable | GACACTGAAGTTACCCAGACACCAAAACACCTGGTCATGGGAAT | 637 |
| 54 | nucleotide | GACAAATAAGAAGTCTTTGAAATGTGAACAACATATGGGGCACA | |
| | | GGGCTATGTATTGGTACAAGCAGAAAGCTAAGAAGCCACCGGAG | |
| | | CTCATGTTTGTCTACAGCTATGAGAAACTCTCTATAAATGAAAGT | |
| | | GTGCCAAGTCGCTTCTCACCTGAATGCCCCAACAGCTCTCTCTTA | |
| | | AACCTTCACCTACACGCCCTGCAGCCAGAAGACTCAGCCCTGTAT | |
| | | CTCTGCGCCAGCAGCCAAGGTCAGGGTCTGAACACTGAAGCTTTC | |
| | | TTTGGACAAGGCACCAGACTCACAGTTGTAG | |
| | Variable-b | DTEVTQTPKHLVMGMTNKKSLKCEQHMGHRAMYWYKQKAKKPP | 638 |
| | | ELMFVYSYEKLSINESVPSRESPECPNSSLLNLHLHALQPEDSALYLC | |
| | | ASSQGQGLNTEAFFGQGTRLTVV | |
| | CDR1-b | MGHRA | 639 |
| | CDR2-b | YSYEKL | 640 |
| | CDR3-b | CASSQGQGLNTEAFF | 641 |
| | FWR1-b | DTEVTQTPKHLVMGMTNKKSLKCEQH | 642 |
| | FWR2-b | MYWYKQKAKKPPELMFV | 643 |
| | FWR3-b | SINESVPSRESPECPNSSLLNLHLHALQPEDSALYL | 644 |
| | FWR4-b | GQGTRLTVV | 246 |
| TCRA- | a-Variable | GGACAAAACATTGACCAGCCCACTGAGATGACAGCTACGGAAGG | 645 |
| 54 | nucleotide | TGCCATTGTCCAGATCAACTGCACGTACCAGACATCTGGGTTCAA | |
| | | CGGGCTGTTCTGGTACCAGCAACATGCTGGCGAAGCACCTACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTG | |
| | | AGAGCATACTCTGGGGCTGGGAGTTACCAACTCACTTTCGGGAAG | |
| | | GGGACCAAACTCTCGGTCATACCAA | |
| | Variable-a | GQNIDQPTEMTATEGAIVQINCTYQTSGFNGLFWYQQHAGEAPTFLS | 646 |
| | | YNVLDGLEEKGRFSSFLSRSKGYSYLLLKELQMKDSASYLCAVRAY | |
| | | SGAGSYQLTFGKGTKLSVIP | |
| | CDR1-a | TSGFNG | 450 |
| | CDR2-a | NVLDGL | 451 |
| | CDR3-a | CAVRAYSGAGSYQLTF | 647 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GKGTKLSVIP | 489 |
| TCRB- | b-Variable | GGTGCTGTCGTCTCTCAACATCCGAGCTGGGTTATCTGTAAGAGT | 648 |
| 55 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGAGATTGGGGCAGGGCCCCAGATA | |
| | | CGCAGTATTTTGGCCCAGGCACCCGGCTGACAGTGCTCG | |
| | Variable-b | GAVVSQHPSWVICKSGTSVKIECRSLDFQATTMFWYRQFPKQSLML | 649 |
| | | MATSNEGSKATYEQGVEKDKFLINHASLTLSTLTVTSAHPEDSSFYIC | |
| | | SARDWGRAPDTQYFGPGTRLTVL | |
| | CDR1-b | DFQATT | 79 |
| | CDR2-b | SNEGSKA | 80 |
| | CDR3-b | CSARDWGRAPDTQYF | 650 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVL | 9 |
| TCRA- | a-Variable | GGAGAGGATGTGGAGCAGAGTCTTTTCCTGAGTGTCCGAGAGGG | 651 |
| 55 | nucleotide | AGACAGCTCCGTTATAAACTGCACTTACACAGACAGCTCCTCCAC | |
| | | CTACTTATACTGGTATAAGCAAGAACCTGGAGCAGGTCTCCAGTT | |
| | | GCTGACGTATATTTTTTCAAATATGGACATGAAACAAGACCAAAG | |
| | | ACTCACTGTTCTATTGAATAAAAAGGATAAACATCTGTCTCTGCG | |
| | | CATTGCAGACACCCAGACTGGGGACTCAGCTATCTACTTCTGTGC | |
| | | AGAGATTAACACCGGTAACCAGTTCTATTTTGGGACAGGGACAA | |
| | | GTTTGACGGTCATTCCAA | |
| | Variable-a | GEDVEQSLFLSVREGDSSVINCTYTDSSSTYLYWYKQEPGAGLQLLT | 652 |
| | | YIFSNMDMKQDQRLTVLLNKKDKHLSLRIADTQTGDSAIYFCAEINT | |
| | | GNQFYFGTGTSLTVIP | |
| | CDR1-a | DSSSTY | 314 |
| | CDR2-a | IFSNMDM | 315 |
| | CDR3-a | CAEINTGNQFYF | 653 |
| | FWR1-a | GEDVEQSLFLSVREGDSSVINCTYT | 317 |
| | FWR2-a | LYWYKQEPGAGLQLLTY | 318 |
| | FWR3-a | KQDQRLTVLLNKKDKHLSLRIADTQTGDSAIYF | 319 |
| | FWR4-a | GTGTSLTVIP | 94 |
| TCRB- | b-Variable | GATTCTGGAGTCACACAAACCCCAAAGCACCTGATCACAGCAAC | 654 |
| 56 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCGCACAAGGGGACAGGGGGGATGGAGCTTTCT | |
| | | TTGGACAAGGCACCAGACTCACAGTTGTAG | |
| | Variable-b | DSGVTQTPKHLITATGQRVTLRCSPRSGDLSVYWYQQSLDQGLQFLI | 655 |
| | | QYYNGEERAKGNILERFSAQQFPDLHSELNLSSLELGDSALYFCASS | |
| | | AQGDRGDGAFFGQGTRLTVV | |
| | CDR1-b | SGDLS | 383 |
| | CDR2-b | YYNGEE | 384 |
| | CDR3-b | CASSAQGDRGDGAFF | 656 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GQGTRLTVV | 246 |
| TCRA- | a-Variable | GCCCAGAAGATAACTCAAACCCAACCAGGAATGTTCGTGCAGGA | 657 |
| 56 | nucleotide | AAAGGAGGCTGTGACTCTGGACTGCACATATGACACCAGTGATC | |
| | | CAAGTTATGGTCTATTCTGGTACAAGCAGCCCAGCAGTGGGGAA | |
| | | ATGATTTTTCTTATTTATCAGGGGTCTTATGACCAGCAAAATGCA | |
| | | ACAGAAGGTCGCTACTCATTGAATTTCCAGAAGGCAAGAAAATC | |
| | | CGCCAACCTTGTCATCTCCGCTTCACAACTGGGGGACTCAGCAAT | |
| | | GTATTTCTGTGCAATGAGAGAGACGGGTAGCAACTATAAACTGA | |
| | | CATTTGGAAAAGGAACTCTCTTAACCGTGAATCCAA | |
| | Variable-a | AQKITQTQPGMFVQEKEAVTLDCTYDTSDPSYGLFWYKQPSSGEMI | 658 |
| | | FLIYQGSYDQQNATEGRYSLNFQKARKSANLVISASQLGDSAMYFC | |
| | | AMRETGSNYKLTFGKGTLLTVNP | |
| | CDR1-a | TSDPSYG | 659 |
| | CDR2-a | QGSYDQQN | 660 |
| | CDR3-a | CAMRETGSNYKLTF | 661 |
| | FWR1-a | AQKITQTQPGMFVQEKEAVTLDCTYD | 406 |
| | FWR2-a | LFWYKQPSSGEMIFLIY | 407 |
| | FWR3-a | ATEGRYSLNFQKARKSANLVISASQLGDSAMYF | 408 |
| | FWR4-a | GKGTLLTVNP | 662 |
| TCRB- | b-Variable | GCTGCTGGAGTCATCCAGTCCCCAAGACATCTGATCAAAGAAAA | 663 |
| 57 | nucleotide | GAGGGAAACAGCCACTCTGAAATGCTATCCTATCCCTAGACACG | |
| | | ACACTGTCTACTGGTACCAGCAGGGTCCAGGTCAGGACCCCCAGT | |
| | | TCCTCATTTCGTTTTATGAAAAGATGCAGAGCGATAAAGGAAGCA | |
| | | TCCCTGATCGATTCTCAGCTCAACAGTTCAGTGACTATCATTCTGA | |
| | | ACTGAACATGAGCTCCTTGGAGCTGGGGGACTCAGCCCTGTACTT | |
| | | CTGTGCCAGCAGCTTAGCAGGGGAGGGTGAAGCTTTCTTTGGACA | |
| | | AGGCACCAGACTCACAGTTGTAG | |
| | Variable-b | AAGVIQSPRHLIKEKRETATLKCYPIPRHDTVYWYQQGPGQDPQFLI | 664 |
| | | SFYEKMQSDKGSIPDRFSAQQFSDYHSELNMSSLELGDSALYFCASS | |
| | | LAGEGEAFFGQGTRLTVV | |
| | CDR1-b | PRHDT | 665 |
| | CDR2-b | FYEKMQ | 666 |
| | CDR3-b | CASSLAGEGEAFF | 667 |
| | FWR1-b | AAGVIQSPRHLIKEKRETATLKCYPI | 668 |
| | FWR2-b | VYWYQQGPGQDPQFLIS | 669 |
| | FWR3-b | SDKGSIPDRFSAQQFSDYHSELNMSSLELGDSALYF | 670 |
| | FWR4-b | GQGTRLTVV | 246 |
| TCRA- | a-Variable | CAGAAGGAGGTGGAGCAGGATCCTGGACCACTCAGTGTTCCAGA | 671 |
| 57 | nucleotide | GGGAGCCATTGTTTCTCTCAACTGCACTTACAGCAACAGTGCTTT | |
| | | TCAATACTTCATGTGGTACAGACAGTATTCCAGAAAAGGCCCTGA | |
| | | GTTGCTGATGTACACATACTCCAGTGGTAACAAAGAAGATGGAA | |
| | | GGTTTACAGCACAGGTCGATAAATCCAGCAAGTATATCTCCTTGT | |
| | | TCATCAGAGACTCACAGCCCAGTGATTCAGCCACCTACCTCTGTG | |
| | | CAGTCGGGGGCGGATCTGAAAAGCTGGTCTTTGGAAAGGGAACG | |
| | | AAACTGACAGTAAACCCAT | |
| | Variable-a | QKEVEQDPGPLSVPEGAIVSLNCTYSNSAFQYFMWYRQYSRKGPEL | 672 |
| | | LMYTYSSGNKEDGRFTAQVDKSSKYISLFIRDSQPSDSATYLCAVGG | |
| | | GSEKLVFGKGTKLTVNP | |
| | CDR1-a | NSAFQY | 673 |
| | CDR2-a | TYSSGN | 674 |
| | CDR3-a | CAVGGGSEKLVF | 675 |
| | FWR1-a | QKEVEQDPGPLSVPEGAIVSLNCTYS | 676 |
| | FWR2-a | FMWYRQYSRKGPELLMY | 677 |
| | FWR3-a | KEDGRFTAQVDKSSKYISLFIRDSQPSDSATYL | 678 |
| | FWR4-a | GKGTKLTVNP | 339 |
| TCRB- | b-Variable | GATGTGAAAGTAACCCAGAGCTCGAGATATCTAGTCAAAAGGAC | 679 |
| 58 | nucleotide | GGGAGAGAAAGTTTTTCTGGAATGTGTCCAGGATATGGACCATG | |
| | | AAAATATGTTCTGGTATCGACAAGACCCAGGTCTGGGGCTACGGC | |
| | | TGATCTATTTCTCATATGATGTTAAAATGAAAGAAAAAGGAGATA | |
| | | TTCCTGAGGGGTACAGTGTCTCTAGAGAGAAGAAGGAGCGCTTCT | |
| | | CCCTGATTCTGGAGTCCGCCAGCACCAACCAGACATCTATGTACC | |
| | | TCTGTGCCAGCAGTTGGACAGAAGAGACCTACGAGCAGTACTTC | |
| | | GGGCCGGGCACCAGGCTCACGGTCACAG | |
| | Variable-b | DVKVTQSSRYLVKRTGEKVFLECVQDMDHENMFWYRQDPGLGLRL | 680 |
| | | IYFSYDVKMKEKGDIPEGYSVSREKKERFSLILESASTNQTSMYLCAS | |
| | | SWTEETYEQYFGPGTRLTVT | |
| | CDR1-b | MDHEN | 199 |
| | CDR2-b | SYDVKM | 200 |
| | CDR3-b | CASSWTEETYEQYF | 681 |
| | FWR1-b | DVKVTQSSRYLVKRTGEKVFLECVQD | 202 |
| | FWR2-b | MFWYRQDPGLGLRLIYF | 203 |
| | FWR3-b | KEKGDIPEGYSVSREKKERFSLILESASTNQTSMYL | 204 |
| | FWR4-b | GPGTRLTVT | 85 |
| TCRA- | a-Variable | GCTCAGTCAGTGGCTCAGCCGGAAGATCAGGTCAACGTTGCTGA | 682 |
| 58 | nucleotide | AGGGAATCCTCTGACTGTGAAATGCACCTATTCAGTCTCTGGAAA | |
| | | CCCTTATCTTTTTTGGTATGTTCAATACCCCAACCGAGGCCTCCAG | |
| | | TTCCTTCTGAAATACATCACAGGGGATAACCTGGTTAAAGGCAGC | |
| | | TATGGCTTTGAAGCTGAATTTAACAAGAGCCAAACCTCCTTCCAC | |
| | | CTGAAGAAACCATCTGCCCTTGTGAGCGACTCCGCTTTGTACTTC | |
| | | TGTGCTGTGAGAGCGTACAATAACAATGACATGCGCTTTGGAGCA | |
| | | GGGACCAGACTGACAGTAAAACCAA | |
| | Variable-a | AQSVAQPEDQVNVAEGNPLTVKCTYSVSGNPYLFWYVQYPNRGLQ | 683 |
| | | FLLKYITGDNLVKGSYGFEAEFNKSQTSFHLKKPSALVSDSALYFCA | |
| | | VRAYNNNDMRFGAGTRLTVKP | |
| | CDR1-a | VSGNPY | 427 |
| | CDR2-a | YITGDNLV | 428 |
| | CDR3-a | CAVRAYNNNDMRF | 684 |
| | FWR1-a | AQSVAQPEDQVNVAEGNPLTVKCTYS | 430 |
| | FWR2-a | LFWYVQYPNRGLQFLLK | 431 |
| | FWR3-a | KGSYGFEAEFNKSQTSFHLKKPSALVSDSALYF | 432 |
| | FWR4-a | GAGTRLTVKP | 685 |
| TCRB- | b-Variable | GATGCTGGAGTTATCCAGTCACCCCGGCACGAGGTGACAGAGAT | 686 |
| 59 | nucleotide | GGGACAAGAAGTGACTCTGAGATGTAAACCAATTTCAGGACACG | |
| | | ACTACCTTTTCTGGTACAGACAGACCATGATGCGGGGACTGGAGT | |
| | | TGCTCATTTACTTTAACAACAACGTTCCGATAGATGATTCAGGGA | |
| | | TGCCCGAGGATCGATTCTCAGCTAAGATGCCTAATGCATCATTCT | |
| | | CCACTCTGAAGATCCAGCCCTCAGAACCCAGGGACTCAGCTGTGT | |
| | | ACTTCTGTGCCAGCAGTTTAGGGACAGGTAGTGAGCAGTTCTTCG | |
| | | GGCCAGGGACACGGCTCACCGTGCTAG | |
| | Variable-b | DAGVIQSPRHEVTEMGQEVTLRCKPISGHDYLFWYRQTMMRGLELL | 687 |
| | | IYFNNNVPIDDSGMPEDRFSAKMPNASFSTLKIQPSEPRDSAVYFCAS | |
| | | SLGTGSEQFFGPGTRLTVL | |
| | CDR1-b | SGHDY | 468 |
| | CDR2-b | FNNNVP | 469 |
| | CDR3-b | CASSLGTGSEQFF | 688 |
| | FWR1-b | DAGVIQSPRHEVTEMGQEVTLRCKPI | 471 |
| | FWR2-b | LFWYRQTMMRGLELLIY | 472 |
| | FWR3-b | IDDSGMPEDRFSAKMPNASFSTLKIQPSEPRDSAVYF | 473 |
| | FWR4-b | GPGTRLTVL | 9 |
| TCRA- | a-Variable | AAACAGGAGGTGACACAGATTCCTGCAGCTCTGAGTGTCCCAGA | 689 |
| 59 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTACGTACCATTTCAGATGGCCAGAAGCTGCTCTTTGCAAG | |
| | | GGGGACCATGTTAAAGGTGGATCTTA | |
| | Variable-a | KQEVTQIPAALSVPEGENLVLNCSFTDSAIYNLQWFRQDPGKGLTSL | 690 |
| | | LLIQSSQREQTSGRLNASLDKSSGRSTLYIAASQPGDSATYLCAVRTI | |
| | | SDGQKLLFARGTMLKVDL | |
| | CDR1-a | DSAIYN | 118 |
| | CDR2-a | IQSSQRE | 119 |
| | CDR3-a | CAVRTISDGQKLLF | 691 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | ARGTMLKVDL | 392 |
| TCRB- | b-Variable | GCTGCTGGAGTCATCCAGTCCCCAAGACATCTGATCAAAGAAAA | 692 |
| 60 | nucleotide | GAGGGAAACAGCCACTCTGAAATGCTATCCTATCCCTAGACACG | |
| | | ACACTGTCTACTGGTACCAGCAGGGTCCAGGTCAGGACCCCCAGT | |
| | | TCCTCATTTCGTTTTATGAAAAGATGCAGAGCGATAAAGGAAGCA | |
| | | TCCCTGATCGATTCTCAGCTCAACAGTTCAGTGACTATCATTCTGA | |
| | | ACTGAACATGAGCTCCTTGGAGCTGGGGGACTCAGCCCTGTACTT | |
| | | CTGTGCCAGCCGGATACGGGGGGGAATGAACACTGAAGCTTTCTT | |
| | | TGGACAAGGCACCAGACTCACAGTTGTAG | |
| | Variable-b | AAGVIQSPRHLIKEKRETATLKCYPIPRHDTVYWYQQGPGQDPQFLI | 693 |
| | | SFYEKMQSDKGSIPDRFSAQQFSDYHSELNMSSLELGDSALYFCASRI | |
| | | RGGMNTEAFFGQGTRLTVV | |
| | CDR1-b | PRHDT | 665 |
| | CDR2-b | FYEKMQ | 666 |
| | CDR3-b | CASRIRGGMNTEAFF | 694 |
| | FWR1-b | AAGVIQSPRHLIKEKRETATLKCYPI | 668 |
| | FWR2-b | VYWYQQGPGQDPQFLIS | 669 |
| | FWR3-b | SDKGSIPDRFSAQQFSDYHSELNMSSLELGDSALYF | 670 |
| | FWR4-b | GQGTRLTVV | 246 |
| TCRA- | a-Variable | GCTCAGTCAGTGGCTCAGCCGGAAGATCAGGTCAACGTTGCTGA | 695 |
| 60 | nucleotide | AGGGAATCCTCTGACTGTGAAATGCACCTATTCAGTCTCTGGAAA | |
| | | CCCTTATCTTTTTTGGTATGTTCAATACCCCAACCGAGGCCTCCAG | |
| | | TTCCTTCTGAAATACATCACAGGGGATAACCTGGTTAAAGGCAGC | |
| | | TATGGCTTTGAAGCTGAATTTAACAAGAGCCAAACCTCCTTCCAC | |
| | | CTGAAGAAACCATCTGCCCTTGTGAGCGACTCCGCTTTGTACTTC | |
| | | TGTGCTGTGAGAGCTCCTCTTTCAGATGGCCAGAAGCTGCTCTTT | |
| | | GCAAGGGGAACCATGTTAAAGGTGGATCTTA | |
| | Variable-a | AQSVAQPEDQVNVAEGNPLTVKCTYSVSGNPYLFWYVQYPNRGLQ | 696 |
| | | FLLKYITGDNLVKGSYGFEAEFNKSQTSFHLKKPSALVSDSALYFCA | |
| | | VRAPLSDGQKLLFARGTMLKVDL | |
| | CDR1-a | VSGNPY | 427 |
| | CDR2-a | YITGDNLV | 428 |
| | CDR3-a | CAVRAPLSDGQKLLF | 697 |
| | FWR1-a | AQSVAQPEDQVNVAEGNPLTVKCTYS | 430 |
| | FWR2-a | LFWYVQYPNRGLQFLLK | 431 |
| | FWR3-a | KGSYGFEAEFNKSQTSFHLKKPSALVSDSALYF | 432 |
| | FWR4-a | ARGTMLKVDL | 392 |
| TCRB- | b-Variable | GGTGCTGTCGTCTCTCAACATCCGAGCTGGGTTATCTGTAAGAGT | 698 |
| 61 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGAGACAGGACTAGCGGGGGGAGC | |
| | | TCCTACGAGCAGTACTTCGGGCCGGGCACCAGGCTCACGGTCACA | |
| | | G | |
| | Variable-b | GAVVSQHPSWVICKSGTSVKIECRSLDFQATTMFWYRQFPKQSLML | 699 |
| | | MATSNEGSKATYEQGVEKDKFLINHASLTLSTLTVTSAHPEDSSFYIC | |
| | | SARDRTSGGSSYEQYFGPGTRLTVT | |
| | CDR1-b | DFQATT | 79 |
| | CDR2-b | SNEGSKA | 80 |
| | CDR3-b | CSARDRTSGGSSYEQYF | 700 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVT | 85 |
| TCRA- | a-Variable | CTTGCTAAGACCACCCAGCCCATCTCCATGGACTCATATGAAGGA | 701 |
| 61 | nucleotide | CAAGAAGTGAACATAACCTGTAGCCACAACAACATTGCTACAAA | |
| | | TGATTATATCACGTGGTACCAACAGTTTCCCAGCCAAGGACCACG | |
| | | ATTTATTATTCAAGGATACAAGACAAAAGTTACAAACGAAGTGG | |
| | | CCTCCCTGTTTATCCCTGCCGACAGAAAGTCCAGCACTCTGAGCC | |
| | | TGCCCCGGGTTTCCCTGAGCGACACTGCTGTGTACTACTGCCTCG | |
| | | TGGGTTCCCTCAACAAACTGGTCTTTGGCGCAGGAACCATTCTGA | |
| | | GAGTCAAGTCCT | |
| | Variable-a | LAKTTQPISMDSYEGQEVNITCSHNNIATNDYITWYQQFPSQGPRFII | 702 |
| | | QGYKTKVTNEVASLFIPADRKSSTLSLPRVSLSDTAVYYCLVGSLNK | |
| | | LVFGAGTILRVKS | |
| | CDR1-a | NIATNDY | 594 |
| | CDR2-a | GYKTK | 595 |
| | CDR3-a | CLVGSLNKLVF | 703 |
| | FWR1-a | LAKTTQPISMDSYEGQEVNITCSHN | 597 |
| | FWR2-a | ITWYQQFPSQGPRFIIQ | 598 |
| | FWR3-a | VTNEVASLFIPADRKSSTLSLPRVSLSDTAVYY | 599 |
| | FWR4-a | GAGTILRVKS | 18 |
| TCRB- | b-Variable | GAAACGGGAGTTACGCAGACACCAAGACACCTGGTCATGGGAAT | 704 |
| 62 | nucleotide | GACAAATAAGAAGTCTTTGAAATGTGAACAACATCTGGGTCATA | |
| | | ACGCTATGTATTGGTACAAGCAAAGTGCTAAGAAGCCACTGGAG | |
| | | CTCATGTTTGTCTACAGTCTTGAAGAACGGGTTGAAAACAACAGT | |
| | | GTGCCAAGTCGCTTCTCACCTGAATGCCCCAACAGCTCTCACTTA | |
| | | TTCCTTCACCTACACACCCTGCAGCCAGAAGACTCGGCCCTGTAT | |
| | | CTCTGCGCCAGCAGCCAATGGGGGGGCGGGAGCTCCTACAATGA | |
| | | GCAGTTCTTCGGGCCAGGGACACGGCTCACCGTGCTAG | |
| | Variable-b | ETGVTQTPRHLVMGMTNKKSLKCEQHLGHNAMYWYKQSAKKPLE | 705 |
| | | LMFVYSLEERVENNSVPSRFSPECPNSSHLFLHLHTLQPEDSALYLCA | |
| | | SSQWGGGSSYNEQFFGPGTRLTVL | |
| | CDR1-b | LGHNA | 169 |
| | CDR2-b | YSLEER | 706 |
| | CDR3-b | CASSQWGGGSSYNEQFF | 707 |
| | FWR1-b | ETGVTQTPRHLVMGMTNKKSLKCEQH | 172 |
| | FWR2-b | MYWYKQSAKKPLELMFV | 173 |
| | FWR3-b | VENNSVPSRFSPECPNSSHLFLHLHTLQPEDSALYL | 708 |
| | FWR4-b | GPGTRLTVL | 9 |
| TCRA- | a-Variable | GGAATACAAGTGGAGCAGAGTCCTCCAGACCTGATTCTCCAGGA | 709 |
| 62 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTGATT | |
| | | ACCTCAGGAACCTACAAATACATCTTTGGAACAGGCACCAGGCT | |
| | | GAAGGTTTTAGCAA | |
| | Variable-a | GIQVEQSPPDLILQEGANSTLRCNFSDSVNNLQWFHQNPWGQLINLF | 710 |
| | | YIPSGTKQNGRLSATTVATERYSLLYISSSQTTDSGVYFCAVITSGTY | |
| | | KYIFGTGTRLKVLA | |
| | CDR1-a | DSVNN | 506 |
| | CDR2-a | IPSGT | 507 |
| | CDR3-a | CAVITSGTYKYIF | 711 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GTGTRLKVLA | 587 |
| TCRB- | b-Variable | AATGCTGGTGTCACTCAGACCCCAAAATTCCAGGTCCTGAAGACA | 712 |
| 63 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTACGACCGGGACACTCTACGAGCAGTACTTCGG | |
| | | GCCGGGCACCAGGCTCACGGTCACAG | |
| | Variable-b | NAGVTQTPKFQVLKTGQSMTLQCAQDMNHEYMSWYRQDPGMGLR | 713 |
| | | LIHYSVGAGITDQGEVPNGYNVSRSTTEDFPLRLLSAAPSQTSVYFCA | |
| | | SSTTGTLYEQYFGPGTRLTVT | |
| | CDR1-b | MNHEY | 252 |
| | CDR2-b | SVGAGI | 253 |
| | CDR3-b | CASSTTGTLYEQYF | 714 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GPGTRLTVT | 85 |
| TCRA- | a-Variable | GGAGAGAATGTGGAGCAGCATCCTTCAACCCTGAGTGTCCAGGA | 715 |
| 63 | nucleotide | GGGAGACAGCGCTGTTATCAAGTGTACTTATTCAGACAGTGCCTC | |
| | | AAACTACTTCCCTTGGTATAAGCAAGAACTTGGAAAAAGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCAACTGGGGGGTATTCAGGAGGAGGTGCTGACGGACTC | |
| | | ACCTTTGGCAAAGGGACTCATCTAATCATCCAGCCCT | |
| | Variable-a | GENVEQHPSTLSVQEGDSAVIKCTYSDSASNYFPWYKQELGKRPQLI | 716 |
| | | IDIRSNVGEKKDQRIA VTLNKTAKHFSLHITETQPEDSAVYFCAATGG | |
| | | YSGGGADGLTFGKGTHLIIQP | |
| | CDR1-a | DSASNY | 207 |
| | CDR2-a | IRSNVGE | 208 |
| | CDR3-a | CAATGGYSGGGADGLTF | 717 |
| | FWR1-a | GENVEQHPSTLSVQEGDSAVIKCTYS | 210 |
| | FWR2-a | FPWYKQELGKRPQLIID | 211 |
| | FWR3-a | KKDQRIAVTLNKTAKHFSLHITETQPEDSAVYF | 212 |
| | FWR4-a | GKGTHLIIQP | 363 |
| TCRB- | b-Variable | GACGCTGGAGTCACCCAAAGTCCCACACACCTGATCAAAACGAG | 718 |
| 64 | nucleotide | AGGACAGCAAGTGACTCTGAGATGCTCTCCTAAGTCTGGGCATGA | |
| | | CACTGTGTCCTGGTACCAACAGGCCCTGGGTCAGGGGCCCCAGTT | |
| | | TATCTTTCAGTATTATGAGGAGGAAGAGAGACAGAGAGGCAACT | |
| | | TCCCTGATCGATTCTCAGGTCACCAGTTCCCTAACTATAGCTCTGA | |
| | | GCTGAATGTGAACGCCTTGTTGCTGGGGGACTCGGCCCTCTATCT | |
| | | CTGTGCCAGCAGCTCTACCGGGACAGCCTCCTACAATGAGCAGTT | |
| | | CTTCGGGCCAGGGACACGGCTCACCGTGCTAG | |
| | Variable-b | DAGVTQSPTHLIKTRGQQVTLRCSPKSGHDTVSWYQQALGQGPQFIF | 719 |
| | | QYYEEEERQRGNFPDRFSGHQFPNYSSELNVNALLLGDSALYLCASS | |
| | | STGTASYNEQFFGPGTRLTVL | |
| | CDR1-b | SGHDT | 331 |
| | CDR2-b | YYEEEE | 332 |
| | CDR3-b | CASSSTGTASYNEQFF | 720 |
| | FWR1-b | DAGVTQSPTHLIKTRGQQVTLRCSPK | 334 |
| | FWR2-b | VSWYQQALGQGPQFIFQ | 131 |
| | FWR3-b | RQRGNFPDRFSGHQFPNYSSELNVNALLLGDSALYL | 335 |
| | FWR4-b | GPGTRLTVL | 9 |
| TCRA- | a-Variable | GGAAATTCAGTGACCCAGATGGAAGGGCCAGTGACTCTCTCAGA | 721 |
| 64 | nucleotide | AGAGGCCTTCCTGACTATAAACTGCACGTACACAGCCACAGGAT | |
| | | ACCCTTCCCTTTTCTGGTATGTCCAATATCCTGGAGAAGGTCTACA | |
| | | GCTCCTCCTGAAAGCCACGAAGGCTGATGACAAGGGAAGCAACA | |
| | | AAGGTTTTGAAGCCACATACCGTAAAGAAACCACTTCTTTCCACT | |
| | | TGGAGAAAGGCTCAGTTCAAGTGTCAGACTCAGCGGTGTACTTCT | |
| | | GTGCTCTGAGTGATCGGAGAGGAAGCTACATACCTACATTTGGAA | |
| | | GAGGAACCAGCCTTATTGTTCATCCGT | |
| | Variable-a | GNSVTQMEGPVTLSEEAFLTINCTYTATGYPSLFWYVQYPGEGLQLL | 722 |
| | | LKATKADDKGSNKGFEATYRKETTSFHLEKGSVQVSDSAVYFCALS | |
| | | DRRGSYIPTFGRGTSLIVHP | |
| | CDR1-a | ATGYPS | 106 |
| | CDR2-a | ATKADDK | 107 |
| | CDR3-a | CALSDRRGSYIPTF | 723 |
| | FWR1-a | GNSVTQMEGPVTLSEEAFLTINCTYT | 724 |
| | FWR2-a | LFWYVQYPGEGLQLLLK | 110 |
| | FWR3-a | GSNKGFEATYRKETTSFHLEKGSVQVSDSAVYF | 111 |
| | FWR4-a | GRGTSLIVHP | 600 |
| TCRB- | b-Variable | GAGACTGGAGTCACCCAAAGTCCCACACACCTGATCAAAACGAG | 725 |
| 65 | nucleotide | AGGACAGCAAGTGACTCTGAGATGCTCTTCTCAGTCTGGGCACAA | |
| | | CACTGTGTCCTGGTACCAACAGGCCCTGGGTCAGGGGCCCCAGTT | |
| | | TATCTTTCAGTATTATAGGGAGGAAGAGAATGGCAGAGGAAACT | |
| | | TCCCTCCTAGATTCTCAGGTCTCCAGTTCCCTAATTATAGCTCTGA | |
| | | GCTGAATGTGAACGCCTTGGAGCTGGACGACTCGGCCCTGTATCT | |
| | | CTGTGCCAGCAGCCCTCCGGGCGGGAGGACCACAGATACGCAGT | |
| | | ATTTTGGCCCAGGCACCCGGCTGACAGTGCTCG | |
| | Variable-b | ETGVTQSPTHLIKTRGQQVTLRCSSQSGHNTVSWYQQALGQGPQFIF | 726 |
| | | QYYREEENGRGNFPPRFSGLQFPNYSSELNVNALELDDSALYLCASS | |
| | | PPGGRTTDTQYFGPGTRLTVL | |
| | CDR1-b | SGHNT | 127 |
| | CDR2-b | YYREEE | 128 |
| | CDR3-b | CASSPPGGRTTDTQYF | 727 |
| | FWR1-b | ETGVTQSPTHLIKTRGQQVTLRCSSQ | 130 |
| | FWR2-b | VSWYQQALGQGPQFIFQ | 131 |
| | FWR3-b | NGRGNFPPRFSGLQFPNYSSELNVNALELDDSALYL | 132 |
| | FWR4-b | GPGTRLTVL | 9 |
| TCRA- | a-Variable | AAACAGGAGGTGACACAGATTCCTGCAGCTCTGAGTGTCCCAGA | 728 |
| 65 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGAGGCCCGCACTCACGGGAGGAGGAAACAAACTCACCT | |
| | | TTGGGACAGGCACTCAGCTAAAAGTGGAACTCA | |
| | Variable-a | KQEVTQIPAALSVPEGENLVLNCSFTDSAIYNLQWFRQDPGKGLTSL | 729 |
| | | LLIQSSQREQTSGRLNASLDKSSGRSTLYIAASQPGDSATYLCAVRPA | |
| | | LTGGGNKLTFGTGTQLKVEL | |
| | CDR1-a | DSAIYN | 118 |
| | CDR2-a | IQSSQRE | 119 |
| | CDR3-a | CAVRPALTGGGNKLTF | 730 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GTGTQLKVEL | 456 |
| TCRB- | b-Variable | GATACTGGAGTCTCCCAGGACCCCAGACACAAGATCACAAAGAG | 731 |
| 66 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCCCCACAAAGATTACTTTCTTTGGACAAG | |
| | | GCACCAGACTCACAGTTGTAG | |
| | Variable-b | DTGVSQDPRHKITKRGQNVTFRCDPISEHNRLYWYRQTLGQGPEFLT | 732 |
| | | YFQNEAQLEKSRLLSDRFSAERPKGSFSTLEIQRTEQGDSAMYLCASS | |
| | | PTKITFFGQGTRLTVV | |
| | CDR1-b | SEHNR | 442 |
| | CDR2-b | FQNEAQ | 443 |
| | CDR3-b | CASSPTKITFF | 733 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GQGTRLTVV | 246 |
| TCRA- | a-Variable | AGTCAACAGGGAGAAGAGGATCCTCAGGCCTTGAGCATCCAGGA | 734 |
| 66 | nucleotide | GGGTGAAAATGCCACCATGAACTGCAGTTACAAAACTAGTATAA | |
| | | ACAATTTACAGTGGTATAGACAAAATTCAGGTAGAGGCCTTGTCC | |
| | | ACCTAATTTTAATACGTTCAAATGAAAGAGAGAAACACAGTGGA | |
| | | AGATTAAGAGTCACGCTTGACACTTCCAAGAAAAGCAGTTCCTTG | |
| | | TTGATCACGGCTTCCCGGGCAGCAGACACTGCTTCTTACTTCTGT | |
| | | GCTACGGACCCGGATTCAGGAAACACACCTCTTGTCTTTGGAAAG | |
| | | GGCACAAGACTTTCTGTGATTGCAA | |
| | Variable-a | SQQGEEDPQALSIQEGENATMNCSYKTSINNLQWYRQNSGRGLVHL | 735 |
| | | ILIRSNEREKHSGRLRVTLDTSKKSSSLLITASRAADTASYFCATDPDS | |
| | | GNTPLVFGKGTRLSVIA | |
| | CDR1-a | TSINN | 552 |
| | CDR2-a | IRSNERE | 553 |
| | CDR3-a | CATDPDSGNTPLVF | 736 |
| | FWR1-a | SQQGEEDPQALSIQEGENATMNCSYK | 555 |
| | FWR2-a | LQWYRQNSGRGLVHLIL | 556 |
| | FWR3-a | KHSGRLRVTLDTSKKSSSLLITASRAADTASYF | 557 |
| | FWR4-a | GKGTRLSVIA | 546 |
| TCRB- | b-Variable | GATACTGGAGTCTCCCAGGACCCCAGACACAAGATCACAAAGAG | 737 |
| 67 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGGCCGGGACTCTTATACGAGCAGTACTTCG | |
| | | GGCCGGGCACCAGGCTCACGGTCACAG | |
| | Variable-b | DTGVSQDPRHKITKRGQNVTFRCDPISEHNRLYWYRQTLGQGPEFLT | 738 |
| | | YFQNEAQLEKSRLLSDRFSAERPKGSFSTLEIQRTEQGDSAMYLCAS | |
| | | RPGLLYEQYFGPGTRLTVT | |
| | CDR1-b | SEHNR | 442 |
| | CDR2-b | FQNEAQ | 443 |
| | CDR3-b | CASRPGLLYEQYF | 739 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GPGTRLTVT | 85 |
| TCRA- | a-Variable | GGAGAGAATGTGGAGCAGCATCCTTCAACCCTGAGTGTCCAGGA | 740 |
| 67 | nucleotide | GGGAGACAGCGCTGTTATCAAGTGTACTTATTCAGACAGTGCCTC | |
| | | AAACTACTTCCCTTGGTATAAGCAAGAACTTGGAAAAGGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCAAGTATGGGAAACACACCTCTTGTCTTTGGAAAGGGC | |
| | | ACAAGACTTTCTGTGATTGCAA | |
| | Variable-a | GENVEQHPSTLSVQEGDSAVIKCTYSDSASNYFPWYKQELGKGPQLI | 741 |
| | | IDIRSNVGEKKDQRIA VTLNKTAKHFSLHITETQPEDSAVYFCAASM | |
| | | GNTPLVFGKGTRLSVIA | |
| | CDR1-a | DSASNY | 207 |
| | CDR2-a | IRSNVGE | 208 |
| | CDR3-a | CAASMGNTPLVF | 742 |
| | FWR1-a | GENVEQHPSTLSVQEGDSAVIKCTYS | 210 |
| | FWR2-a | FPWYKQELGKGPQLIID | 374 |
| | FWR3-a | KKDQRIAVTLNKTAKHFSLHITETQPEDSAVYF | 212 |
| | FWR4-a | GKGTRLSVIA | 546 |
| TCRB- | b-Variable | GGAGCTGGAGTCTCCCAGTCCCCCAGTAACAAGGTCACAGAGAA | 743 |
| 68 | nucleotide | GGGAAAGGATGTAGAGCTCAGGTGTGATCCAATTTCAGGTCATA | |
| | | CTGCCCTTTACTGGTACCGACAGAGGCTGGGGCAGGGCCTGGAGT | |
| | | TTTTAATTTACTTCCAAGGCAACAGTGCACCAGACAAATCAGGGC | |
| | | TGCCCAGTGATCGCTTCTCTGCAGAGAGGACTGGGGAATCCGTCT | |
| | | CCACTCTGACGATCCAGCGCACACAGCAGGAGGACTCGGCCGTG | |
| | | TATCTCTGTGCCAGCAGCCCAACTAGCGGGTACGAGCAGTACTTC | |
| | | GGGCCGGGCACCAGGCTCACGGTCACAG | |
| | Variable-b | GAGVSQSPSNKVTEKGKDVELRCDPISGHTALYWYRQRLGQGLEFL | 744 |
| | | IYFQGNSAPDKSGLPSDRFSAERTGESVSTLTIQRTQQEDSAVYLCAS | |
| | | SPTSGYEQYFGPGTRLTVT | |
| | CDR1-b | SGHTA | 263 |
| | CDR2-b | FQGNSA | 629 |
| | CDR3-b | CASSPTSGYEQYF | 745 |
| | FWR1-b | GAGVSQSPSNKVTEKGKDVELRCDPI | 631 |
| | FWR2-b | LYWYRQRLGQGLEFLIY | 746 |
| | FWR3-b | PDKSGLPSDRFSAERTGESVSTLTIQRTQQEDSAVYL | 747 |
| | FWR4-b | GPGTRLTVT | 85 |
| TCRA- | a-Variable | GGAGAGAATGTGGAGCAGCATCCTTCAACCCTGAGTGTCCAGGA | 748 |
| 68 | nucleotide | GGGAGACAGCGCTGTTATCAAGTGTACTTATTCAGACAGTGCCTC | |
| | | AAACTACTTCCCTTGGTATAAGCAAGAACTTGGAAAAGGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCAAGCTCCTATTATAACCAGGGAGGAAAGCTTATCTTCG | |
| | | GACAGGGAACGGAGTTATCTGTGAAACCCA | |
| | Variable-a | GENVEQHPSTLSVQEGDSAVIKCTYSDSASNYFPWYKQELGKGPQLI | 749 |
| | | IDIRSNVGEKKDQRIA VTLNKTAKHFSLHITETQPEDSAVYFCAASSY | |
| | | YNQGGKLIFGQGTELSVKP | |
| | CDR1-a | DSASNY | 207 |
| | CDR2-a | IRSNVGE | 208 |
| | CDR3-a | CAASSYYNQGGKLIF | 750 |
| | FWR1-a | GENVEQHPSTLSVQEGDSAVIKCTYS | 210 |
| | FWR2-a | FPWYKQELGKGPQLIID | 374 |
| | FWR3-a | KKDQRIAVTLNKTAKHFSLHITETQPEDSAVYF | 212 |
| | FWR4-a | GQGTELSVKP | 409 |
| TCRB- | b-Variable | GATTCTGGAGTCACACAAACCCCAAAGCACCTGATCACAGCAAC | 751 |
| 69 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCGTGGGCGGACAGGGCAATCAGCCCCAGCATT | |
| | | TTGGTGATGGGACTCGACTCTCCATCCTAG | |
| | Variable-b | DSGVTQTPKHLITATGQRVTLRCSPRSGDLSVYWYQQSLDQGLQFLI | 752 |
| | | QYYNGEERAKGNILERFSAQQFPDLHSELNLSSLELGDSALYFCASS | |
| | | VGGQGNQPQHFGDGTRLSIL | |
| | CDR1-b | SGDLS | 383 |
| | CDR2-b | YYNGEE | 384 |
| | CDR3-b | CASSVGGQGNQPQHF | 753 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GDGTRLSIL | 27 |
| TCRA- | a-Variable | GGACAAAACATTGACCAGCCCACTGAGATGACAGCTACGGAAGG | 754 |
| 69 | nucleotide | TGCCATTGTCCAGATCAACTGCACGTACCAGACATCTGGGTTCAA | |
| | | CGGGCTGTTCTGGTACCAGCAACATGCTGGCGAAGCACCCACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTACA | |
| | | TATGCTGGCAACAACCGTAAGCTGATTTGGGGATTGGGAACAAG | |
| | | CCTGGCAGTAAATCCGA | |
| | Variable-a | GQNIDQPTEMTATEGAIVQINCTYQTSGFNGLFWYQQHAGEAPTFLS | 755 |
| | | YNVLDGLEEKGRFSSFLSRSKGYSYLLLKELQMKDSASYLCATYAG | |
| | | NNRKLIWGLGTSLAVNP | |
| | CDR1-a | TSGFNG | 450 |
| | CDR2-a | NVLDGL | 451 |
| | CDR3-a | CATYAGNNRKLIW | 756 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GLGTSLAVNP | 242 |
| TCRB- | b-Variable | GACGCTGGAGTCACCCAAAGTCCCACACACCTGATCAAAACGAG | 757 |
| 70 | nucleotide | AGGACAGCAAGTGACTCTGAGATGCTCTCCTAAGTCTGGGCATGA | |
| | | CACTGTGTCCTGGTACCAACAGGCCCTGGGTCAGGGGCCCCAGTT | |
| | | TATCTTTCAGTATTATGAGGAGGAAGAGAGACAGAGAGGCAACT | |
| | | TCCCTGATCGATTCTCAGGTCACCAGTTCCCTAACTATAGCTCTGA | |
| | | GCTGAATGTGAACGCCTTGTTGCTGGGGGACTCGGCCCTCTATCT | |
| | | CTGTGCCAGCAGCCCATTGGAGTCGAACACCGGGGAGCTGTTTTT | |
| | | TGGAGAAGGCTCTAGGCTGACCGTACTGG | |
| | Variable-b | DAGVTQSPTHLIKTRGQQVTLRCSPKSGHDTVSWYQQALGQGPQFIF | 758 |
| | | QYYEEEERQRGNFPDRFSGHQFPNYSSELNVNALLLGDSALYLCASS | |
| | | PLESNTGELFFGEGSRLTVL | |
| | CDR1-b | SGHDT | 331 |
| | CDR2-b | YYEEEE | 332 |
| | CDR3-b | CASSPLESNTGELFF | 759 |
| | FWR1-b | DAGVTQSPTHLIKTRGQQVTLRCSPK | 334 |
| | FWR2-b | VSWYQQALGQGPQFIFQ | 131 |
| | FWR3-b | RQRGNFPDRFSGHQFPNYSSELNVNALLLGDSALYL | 335 |
| | FWR4-b | GEGSRLTVL | 63 |
| TCRA- | a-Variable | ATACTGAACGTGGAACAAAGTCCTCAGTCACTGCATGTTCAGGAG | 760 |
| 70 | nucleotide | GGAGACAGCACCAATTTCACCTGCAGCTTCCCTTCCAGCAATTTT | |
| | | TATGCCTTACACTGGTACAGATGGGAAACTGCAAAAAGCCCCGA | |
| | | GGCCTTGTTTGTAATGACTTTAAATGGGGATGAAAAGAAGAAAG | |
| | | GACGAATAAGTGCCACTCTTAATACCAAGGAGGGTTACAGCTATT | |
| | | TGTACATCAAAGGATCCCAGCCTGAAGACTCAGCCACATACCTCT | |
| | | GTGCCCTCCTTAACACCGGTAACCAGTTCTATTTTGGGACAGGGA | |
| | | CAAGTTTGACGGTCATTCCAA | |
| | Variable-a | ILNVEQSPQSLHVQEGDSTNFTCSFPSSNFYALHWYRWETAKSPEAL | 761 |
| | | FVMTLNGDEKKKGRISATLNTKEGYSYLYIKGSQPEDSATYLCALLN | |
| | | TGNQFYFGTGTSLTVIP | |
| | CDR1-a | SSNFYA | 762 |
| | CDR2-a | MTLNGDE | 763 |
| | CDR3-a | CALLNTGNQFYF | 764 |
| | FWR1-a | ILNVEQSPQSLHVQEGDSTNFTCSFP | 765 |
| | FWR2-a | LHWYRWETAKSPEALFV | 766 |
| | FWR3-a | KKKGRISATLNTKEGYSYLYIKGSQPEDSATYL | 767 |
| | FWR4-a | GTGTSLTVIP | 94 |
| TCRB- | b-Variable | GAAACGGGAGTTACGCAGACACCAAGACACCTGGTCATGGGAAT | 768 |
| 71 | nucleotide | GACAAATAAGAAGTCTTTGAAATGTGAACAACATCTGGGGCATA | |
| | | ACGCTATGTATTGGTACAAGCAAAGTGCTAAGAAGCCACTGGAG | |
| | | CTCATGTTTGTCTACAACTTTAAAGAACAGACTGAAAACAACAGT | |
| | | GTGCCAAGTCGCTTCTCACCTGAATGCCCCAACAGCTCTCACTTA | |
| | | TTCCTTCACCTACACACCCTGCAGCCAGAAGACTCGGCCCTGTAT | |
| | | CTCTGTGCCAGCAGCCCAGGGACAGGGGGCGCGGCCGGGGAGCT | |
| | | GTTTTTTGGAGAAGGCTCTAGGCTGACCGTACTGG | |
| | Variable-b | ETGVTQTPRHLVMGMTNKKSLKCEQHLGHNAMYWYKQSAKKPLE | 769 |
| | | LMFVYNFKEQTENNSVPSRFSPECPNSSHLFLHLHTLQPEDSALYLC | |
| | | ASSPGTGGAAGELFFGEGSRLTVL | |
| | CDR1-b | LGHNA | 169 |
| | CDR2-b | YNFKEQ | 170 |
| | CDR3-b | CASSPGTGGAAGELFF | 770 |
| | FWR1-b | ETGVTQTPRHLVMGMTNKKSLKCEQH | 172 |
| | FWR2-b | MYWYKQSAKKPLELMFV | 173 |
| | FWR3-b | TENNSVPSRFSPECPNSSHLFLHLHTLQPEDSALYL | 174 |
| | FWR4-b | GEGSRLTVL | 63 |
| TCRA- | a-Variable | GGAGAGGATGTGGAGCAGAGTCTTTTCCTGAGTGTCCGAGAGGG | 771 |
| 71 | nucleotide | AGACAGCTCCGTTATAAACTGCACTTACACAGACAGCTCCTCCAC | |
| | | CTACTTATACTGGTATAAGCAAGAACCTGGAGCAGGTCTCCAGTT | |
| | | GCTGACGTATATTTTTTCAAATATGGACATGAAACAAGACCAAAG | |
| | | ACTCACTGTTCTATTGAATAAAAAGGATAAACATCTGTCTCTGCG | |
| | | CATTGCAGACACCCAGACTGGGGACTCAGCTATCTACTTCTGTGC | |
| | | GGCTATAGGATTTTCTGGTGGCTACAATAAGCTGATTTTTGGAGC | |
| | | AGGGACCAGGCTGGCTGTACACCCAT | |
| | Variable-a | GEDVEQSLFLSVREGDSSVINCTYTDSSSTYLYWYKQEPGAGLQLLT | 772 |
| | | YIFSNMDMKQDQRLTVLLNKKDKHLSLRIADTQTGDSAIYFCAAIGF | |
| | | SGGYNKLIFGAGTRLAVHP | |
| | CDR1-a | DSSSTY | 314 |
| | CDR2-a | IFSNMDM | 315 |
| | CDR3-a | CAAIGFSGGYNKLIF | 773 |
| | FWR1-a | GEDVEQSLFLSVREGDSSVINCTYT | 317 |
| | FWR2-a | LYWYKQEPGAGLQLLTY | 318 |
| | FWR3-a | KQDQRLTVLLNKKDKHLSLRIADTQTGDSAIYF | 319 |
| | FWR4-a | GAGTRLAVHP | 774 |
| TCRB- | b-Variable | GATGGTGGAATCACTCAGTCCCCAAAGTACCTGTTCAGAAAGGA | 775 |
| 72 | nucleotide | AGGACAGAATGTGACCCTGAGTIGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGTATTGCAGGGGCGGACTACGAGCAGTACTT | |
| | | CGGGCCGGGCACCAGGCTCACGGTCACAG | |
| | Variable-b | DGGITQSPKYLFRKEGQNVTLSCEQNLNHDAMYWYRQDPGQGLRLI | 776 |
| | | YYSQIVNDFQKGDIAEGYSVSREKKESFPLTVTSAQKNPTAFYLCAS | |
| | | SIAGADYEQYFGPGTRLTVT | |
| | CDR1-b | LNHDA | 216 |
| | CDR2-b | SQIVND | 217 |
| | CDR3-b | CASSIAGADYEQYF | 777 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVT | 85 |
| TCRA- | a-Variable | GGAGAGAATGTGGAGCAGCATCCTTCAACCCTGAGTGTCCAGGA | 778 |
| 72 | nucleotide | GGGAGACAGCGCTGTTATCAAGTGTACTTATTCAGACAGTGCCTC | |
| | | AAACTACTTCCCTTGGTATAAGCAAGAACTTGGAAAAAGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCACAGCCACCAACGCCAACGAACACCGGTAACCAGTTC | |
| | | TATTTTGGGACAGGGACAAGTTTGACGGTCATTCCAA | |
| | Variable-a | GENVEQHPSTLSVQEGDSAVIKCTYSDSASNYFPWYKQELGKRPQLI | 779 |
| | | IDIRSNVGEKKDQRIA VTLNKTAKHFSLHITETQPEDSAVYFCAAQPP | |
| | | TPTNTGNQFYFGTGTSLTVIP | |
| | CDR1-a | DSASNY | 207 |
| | CDR2-a | IRSNVGE | 208 |
| | CDR3-a | CAAQPPTPTNTGNQFYF | 780 |
| | FWR1-a | GENVEQHPSTLSVQEGDSAVIKCTYS | 210 |
| | FWR2-a | FPWYKQELGKRPQLIID | 211 |
| | FWR3-a | KKDQRIAVTLNKTAKHFSLHITETQPEDSAVYF | 212 |
| | FWR4-a | GTGTSLTVIP | 94 |
| TCRB- | b-Variable | GCTGCTGGAGTCATCCAGTCCCCAAGACATCTGATCAAAGAAAA | 781 |
| 73 | nucleotide | GAGGGAAACAGCCACTCTGAAATGCTATCCTATCCCTAGACACG | |
| | | ACACTGTCTACTGGTACCAGCAGGGTCCAGGTCAGGACCCCCAGT | |
| | | TCCTCATTTCGTTTTATGAAAAGATGCAGAGCGATAAAGGAAGCA | |
| | | TCCCTGATCGATTCTCAGCTCAACAGTTCAGTGACTATCATTCTGA | |
| | | ACTGAACATGAGCTCCTTGGAGCTGGGGGACTCAGCCCTGTACTT | |
| | | CTGTGCCAGCAGACCACAGGGAACTAATGAAAAACTGTTTTTTGG | |
| | | CAGTGGAACCCAGCTCTCTGTCTTGG | |
| | Variable-b | AAGVIQSPRHLIKEKRETATLKCYPIPRHDTVYWYQQGPGQDPQFLI | 782 |
| | | SFYEKMQSDKGSIPDRFSAQQFSDYHSELNMSSLELGDSALYFCASR | |
| | | PQGTNEKLFFGSGTQLSVL | |
| | CDR1-b | PRHDT | 665 |
| | CDR2-b | FYEKMQ | 666 |
| | CDR3-b | CASRPQGTNEKLFF | 783 |
| | FWR1-b | AAGVIQSPRHLIKEKRETATLKCYPI | 668 |
| | FWR2-b | VYWYQQGPGQDPQFLIS | 669 |
| | FWR3-b | SDKGSIPDRFSAQQFSDYHSELNMSSLELGDSALYF | 670 |
| | FWR4-b | GSGTQLSVL | 45 |
| TCRA- | a-Variable | AAACAGGAGGTGACGCAGATTCCTGCAGCTCTGAGTGTCCCAGA | 784 |
| 73 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGGAAACCAGTGGCTCTAGGTTGACCTTTGGGGAAGGAAC | |
| | | ACAGCTCACAGTGAATCCTG | |
| | Variable-a | KQEVTQIPAALSVPEGENLVLNCSFTDSAIYNLQWFRQDPGKGLTSL | 785 |
| | | LLIQSSQREQTSGRLNASLDKSSGRSTLYIAASQPGDSATYLCAVETS | |
| | | GSRLTFGEGTQLTVNP | |
| | CDR1-a | DSAIYN | 118 |
| | CDR2-a | IQSSQRE | 119 |
| | CDR3-a | CAVETSGSRLTF | 786 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GEGTQLTVNP | 787 |
| TCRB- | b-Variable | GGTGCTGGAGTCTCCCAGTCCCCTAGGTACAAAGTCGCAAAGAG | 788 |
| 74 | nucleotide | AGGACAGGATGTAGCTCTCAGGTGTGATCCAATTTCGGGTCATGT | |
| | | ATCCCTTTTTTGGTACCAACAGGCCCTGGGGCAGGGGCCAGAGTT | |
| | | TCTGACTTATTTCCAGAATGAAGCTCAACTAGACAAATCGGGGCT | |
| | | GCCCAGTGATCGCTTCTTTGCAGAAAGGCCTGAGGGATCCGTCTC | |
| | | CACTCTGAAGATCCAGCGCACACAGCAGGAGGACTCCGCCGTGT | |
| | | ATCTCTGTGCCAGCGGAGCTAGCGGGGGGGCTCACACCGGGGAG | |
| | | CTGTTTTTTGGAGAAGGCTCTAGGCTGACCGTACTGG | |
| | Variable-b | GAGVSQSPRYKVAKRGQDVALRCDPISGHVSLFWYQQALGQGPEFL | 789 |
| | | TYFQNEAQLDKSGLPSDRFFAERPEGSVSTLKIQRTQQEDSAVYLCA | |
| | | SGASGGAHTGELFFGEGSRLTVL | |
| | CDR1-b | SGHVS | 275 |
| | CDR2-b | FQNEAQ | 443 |
| | CDR3-b | CASGASGGAHTGELFF | 790 |
| | FWR1-b | GAGVSQSPRYKVAKRGQDVALRCDPI | 791 |
| | FWR2-b | LFWYQQALGQGPEFLTY | 792 |
| | FWR3-b | LDKSGLPSDRFFAERPEGSVSTLKIQRTQQEDSAVYL | 793 |
| | FWR4-b | GEGSRLTVL | 63 |
| TCRA- | a-Variable | GGAGAGGATGTGGAGCAGAGTCTTTTCCTGAGTGTCCGAGAGGG | 794 |
| 74 | nucleotide | AGACAGCTCCGTTATAAACTGCACTTACACAGACAGCTCCTCCAC | |
| | | CTACTTATACTGGTATAAGCAAGAACCTGGAGCAGGTCTCCAGTT | |
| | | GCTGACGTATATTTTTTCAAATATGGACATGAAACAAGACCAAAG | |
| | | ACTCACTGTTCTATTGAATAAAAAGGATAAACATCTGTCTCTGCG | |
| | | CATTGCAGACACCCAGACTGGGGACTCAGCTATCTACTTCTGTGC | |
| | | AGAGAGCGAGGGAGGAAAGCTTATCTTCGGACAGGGAACGGAGT | |
| | | TATCTGTGAAACCCA | |
| | Variable-a | GEDVEQSLFLSVREGDSSVINCTYTDSSSTYLYWYKQEPGAGLQLLT | 795 |
| | | YIFSNMDMKQDQRLTVLLNKKDKHLSLRIADTQTGDSAIYFCAESEG | |
| | | GKLIFGQGTELSVKP | |
| | CDR1-a | DSSSTY | 314 |
| | CDR2-a | IFSNMDM | 315 |
| | CDR3-a | CAESEGGKLIF | 796 |
| | FWR1-a | GEDVEQSLFLSVREGDSSVINCTYT | 317 |
| | FWR2-a | LYWYKQEPGAGLQLLTY | 318 |
| | FWR3-a | KQDQRLTVLLNKKDKHLSLRIADTQTGDSAIYF | 319 |
| | FWR4-a | GQGTELSVKP | 409 |
| TCRB- | b-Variable | GATGTGAAAGTAACCCAGAGCTCGAGATATCTAGTCAAAAGGAC | 797 |
| 75 | nucleotide | GGGAGAGAAAGTTTTTCTGGAATGTGTCCAGGATATGGACCATG | |
| | | AAAATATGTTCTGGTATCGACAAGACCCAGGTCTGGGGCTACGGC | |
| | | TGATCTATTTCTCATATGATGTTAAAATGAAAGAAAAAGGAGATA | |
| | | TTCCTGAGGGGTACAGTGTCTCTAGAGAGAAGAAGGAGCGCTTCT | |
| | | CCCTGATTCTGGAGTCCGCCAGCACCAACCAGACATCTATGTACC | |
| | | TCTGTGCCAGCAGACCTTTGAGGAGCAATCAGCCCCAGCATTTTG | |
| | | GTGATGGGACTCGACTCTCCATCCTAG | |
| | Variable-b | DVKVTQSSRYLVKRTGEKVFLECVQDMDHENMFWYRQDPGLGLRL | 798 |
| | | IYFSYDVKMKEKGDIPEGYSVSREKKERFSLILESASTNQTSMYLCAS | |
| | | RPLRSNQPQHFGDGTRLSIL | |
| | CDR1-b | MDHEN | 199 |
| | CDR2-b | SYDVKM | 200 |
| | CDR3-b | CASRPLRSNQPQHF | 799 |
| | FWR1-b | DVKVTQSSRYLVKRTGEKVFLECVQD | 202 |
| | FWR2-b | MFWYRQDPGLGLRLIYF | 203 |
| | FWR3-b | KEKGDIPEGYSVSREKKERFSLILESASTNQTSMYL | 204 |
| | FWR4-b | GDGTRLSIL | 27 |
| TCRA- | a-Variable | GGACAAAACATTGACCAGCCCACTGAGATGACAGCTACGGAAGG | 800 |
| 75 | nucleotide | TGCCATTGTCCAGATCAACTGCACGTACCAGACATCTGGGTTCAA | |
| | | CGGGCTGTTCTGGTACCAGCAACATGCTGGCGAAGCACCCACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTG | |
| | | AGAGAAGCTAATAATGCAGGCAACATGCTCACCTTTGGAGGGGG | |
| | | AACAAGGTTAATGGTCAAACCCC | |
| | Variable-a | GQNIDQPTEMTATEGAIVQINCTYQTSGFNGLFWYQQHAGEAPTFLS | 801 |
| | | YNVLDGLEEKGRESSFLSRSKGYSYLLLKELQMKDSASYLCAVREA | |
| | | NNAGNMLTFGGGTRLMVKP | |
| | CDR1-a | TSGFNG | 450 |
| | CDR2-a | NVLDGL | 451 |
| | CDR3-a | CAVREANNAGNMLTF | 802 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GGGTRLMVKP | 141 |
| TCRB- | b-Variable | AGTGCTGTCATCTCTCAAAAGCCAAGCAGGGATATCTGTCAACGT | 803 |
| 76 | nucleotide | GGAACCTCCCTGACGATCCAGTGTCAAGTCGATAGCCAAGTCACC | |
| | | ATGATGTTCTGGTACCGTCAGCAACCTGGACAGAGCCTGACACTG | |
| | | ATCGCAACTGCAAATCAGGGCTCTGAGGCCACATATGAGAGTGG | |
| | | ATTTGTCATTGACAAGTTTCCCATCAGCCGCCCAAACCTAACATT | |
| | | CTCAACTCTGACTGTGAGCAACATGAGCCCTGAAGACAGCAGCA | |
| | | TATATCTCTGCAGCGTTGAGTATGAGCAGTTCTTCGGGCCAGGGA | |
| | | CACGGCTCACCGTGCTAG | |
| | Variable-b | SAVISQKPSRDICQRGTSLTIQCQVDSQVTMMFWYRQQPGQSLTLIA | 804 |
| | | TANQGSEATYESGFVIDKFPISRPNLTFSTLTVSNMSPEDSSIYLCSVE | |
| | | YEQFFGPGTRLTVL | |
| | CDR1-b | SQVTM | 97 |
| | CDR2-b | ANQGSEA | 98 |
| | CDR3-b | CSVEYEQFF | 805 |
| | FWR1-b | SAVISQKPSRDICQRGTSLTIQCQVD | 100 |
| | FWR2-b | MFWYRQQPGQSLTLIAT | 101 |
| | FWR3-b | TYESGFVIDKFPISRPNLTFSTLTVSNMSPEDSSIYL | 102 |
| | FWR4-b | GPGTRLTVL | 9 |
| TCRA- | a-Variable | CAGAAGGAGGTGGAGCAGAATTCTGGACCCCTCAGTGTTCCAGA | 806 |
| 76.1 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTTCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAACTCGATTGAAAACTTTGGAAATGAGAAATTAACCTTTG | |
| | | GGACTGGAACAAGACTCACCATCATACCCA | |
| | Variable-a | QKEVEQNSGPLSVPEGAIASLNCTYSDRGSQSFFWYRQYSGKSPELI | 807 |
| | | MFIYSNGDKEDGRFTAQLNKASQYVSLLIRDSQPSDSATYLCAVNSI | |
| | | ENFGNEKLTFGTGTRLTIIP | |
| | CDR1-a | DRGSQS | 12 |
| | CDR2-a | IYSNGD | 13 |
| | CDR3-a | CAVNSIENFGNEKLTF | 808 |
| | FWR1-a | QKEVEQNSGPLSVPEGAIASLNCTYS | 15 |
| | FWR2-a | FFWYRQYSGKSPELIMF | 16 |
| | FWR3-a | KEDGRFTAQLNKASQYVSLLIRDSQPSDSATYL | 17 |
| | FWR4-a | GTGTRLTIIP | 292 |
| TCRA- | a-Variable | GCTCAGACAGTCACTCAGTCTCAACCAGAGATGTCTGTGCAGGAG | 809 |
| 76.2 | nucleotide | GCAGAGACCGTGACCCTGAGCTGCACATATGACACCAGTGAGAG | |
| | | TGATTATTATTTATTCTGGTACAAGCAGCCTCCCAGCAGGCAGAT | |
| | | GATTCTCGTTATTCGCCAAGAAGCTTATAAGCAACAGAATGCAAC | |
| | | AGAGAATCGTTTCTCTGTGAACTTCCAGAAAGCAGCCAAATCCTT | |
| | | CAGTCTCAAGATCTCAGACTCACAGCTGGGGGATGCCGCGATGTA | |
| | | TTTCTGTGCTTATAGGATCTCCTGGTCAAATTCCGGGTATGCACTC | |
| | | AACTTCGGCAAAGGCACCTCGCTGTTGGTCACACCCC | |
| | Variable-a | AQTVTQSQPEMSVQEAETVTLSCTYDTSESDYYLFWYKQPPSRQMI | 810 |
| | | LVIRQEAYKQQNATENRFSVNFQKAAKSFSLKISDSQLGDAAMYFC | |
| | | AYRISWSNSGYALNFGKGTSLLVTP | |
| | CDR1-a | TSESDYY | 286 |
| | CDR2-a | QEAYKQQN | 287 |
| | CDR3-a | CAYRISWSNSGYALNF | 811 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDAAMYF | 291 |
| | FWR4-a | GKGTSLLVTP | 812 |
| TCRB- | b-Variable | AATGCCGGCGTCATGCAGAACCCAAGACACCTGGTCAGGAGGAG | 813 |
| 77 | nucleotide | GGGACAGGAGGCAAGACTGAGATGCAGCCCAATGAAAGGACAC | |
| | | AGTCATGTTTACTGGTATCGGCAGCTCCCAGAGGAAGGTCTGAAA | |
| | | TTCATGGTTTATCTCCAGAAAGAAAATATCATAGATGAGTCAGGA | |
| | | ATGCCAAAGGAACGATTTTCTGCTGAATTTCCCAAAGAGGGCCCC | |
| | | AGCATCCTGAGGATCCAGCAGGTAGTGCGAGGAGATTCGGCAGC | |
| | | TTATTTCTGTGCCAGCTCACAAGACAGGGCCGGGGCCAACGTCCT | |
| | | GACTTTCGGGGCCGGCAGCAGGCTGACCGTGCTGG | |
| | Variable-b | NAGVMQNPRHLVRRRGQEARLRCSPMKGHSHVYWYRQLPEEGLKF | 814 |
| | | MVYLQKENIIDESGMPKERFSAEFPKEGPSILRIQQVVRGDSAAYFCA | |
| | | SSQDRAGANVLTFGAGSRLTVL | |
| | CDR1-b | KGHSH | 144 |
| | CDR2-b | LQKENI | 145 |
| | CDR3-b | CASSQDRAGANVLTF | 815 |
| | FWR1-b | NAGVMQNPRHLVRRRGQEARLRCSPM | 147 |
| | FWR2-b | VYWYRQLPEEGLKFMVY | 148 |
| | FWR3-b | IDESGMPKERFSAEFPKEGPSILRIQQVVRGDSAAYF | 149 |
| | FWR4-b | GAGSRLTVL | 816 |
| TCRA- | a-Variable | GATGCTAAGACCACCCAGCCCCCCTCCATGGATTGCGCTGAAGGA | 817 |
| 77 | nucleotide | AGAGCTGCAAACCTGCCTTGTAATCACTCTACCATCAGTGGAAAT | |
| | | GAGTATGTGTATTGGTATCGACAGATTCACTCCCAGGGGCCACAG | |
| | | TATATCATTCATGGTCTAAAAAACAATGAAACCAATGAAATGGCC | |
| | | TCTCTGATCATCACAGAAGACAGAAAGTCCAGCACCTTGATCCTG | |
| | | CCCCACGCTACGCTGAGAGACACTGCTGTGTACTATTGCATCGTC | |
| | | CGAGGAGGTGCTGACGGACTCACCTTTGGCAAAGGGACTCATCT | |
| | | AATCATCCAGCCCT | |
| | Variable-a | DAKTTQPPSMDCAEGRAANLPCNHSTISGNEYVYWYRQIHSQGPQY | 818 |
| | | IIHGLKNNETNEMASLIITEDRKSSTLILPHATLRDTAVYYCIVRGGA | |
| | | DGLTFGKGTHLIIQP | |
| | CDR1-a | TISGNEY | 88 |
| | CDR2-a | GLKNN | 89 |
| | CDR3-a | CIVRGGADGLTF | 819 |
| | FWR1-a | DAKTTQPPSMDCAEGRAANLPCNHS | 439 |
| | FWR2-a | VYWYRQIHSQGPQYIIH | 92 |
| | FWR3-a | ETNEMASLIITEDRKSSTLILPHATLRDTAVYY | 93 |
| | FWR4-a | GKGTHLIIQP | 363 |
| TCRB- | b-Variable | GATACTGGAGTCTCCCAGGACCCCAGACACAAGATCACAAAGAG | 820 |
| 78 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAATCGGCAACGAGATAATTCACCCCTCCACT | |
| | | TTGGGAATGGGACCAGGCTCACTGTGACAG | |
| | Variable-b | DTGVSQDPRHKITKRGQNVTFRCDPISEHNRLYWYRQTLGQGPEFLT | 821 |
| | | YFQNEAQLEKSRLLSDRFSAERPKGSFSTLEIQRTEQGDSAMYLCAS | |
| | | NRQRDNSPLHFGNGTRLTVT | |
| | CDR1-b | SEHNR | 442 |
| | CDR2-b | FQNEAQ | 443 |
| | CDR3-b | CASNRQRDNSPLHF | 822 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GNGTRLTVT | 150 |
| TCRA- | a-Variable | GCCCAGAAGATAACTCAAACCCAACCAGGAATGTTCGTGCAGGA | 823 |
| 78 | nucleotide | AAAGGAGGCTGTGACTCTGGACTGCACATATGACACCAGTGATC | |
| | | AAAGTTATGGTCTATTCTGGTACAAGCAGCCCAGCAGTGGGGAA | |
| | | ATGATTTTTCTTATTTATCAGGGGTCTTATGACGAGCAAAATGCA | |
| | | ACAGAAGGTCGCTACTCATTGAATTTCCAGAAGGCAAGAAAATC | |
| | | CGCCAACCTTGTCATCTCCGCTTCACAACTGGGGGACTCAGCAAT | |
| | | GTATTTCTGTGCAATGAGAGAGTGGTTTTCAGATGGCCAGAAGCT | |
| | | GCTCTTTGCAAGGGGGACCATGTTAAAGGTGGATCTTA | |
| | Variable-a | AQKITQTQPGMFVQEKEAVTLDCTYDTSDQSYGLFWYKQPSSGEMI | 824 |
| | | FLIYQGSYDEQNATEGRYSLNFQKARKSANLVISASQLGDSAMYFC | |
| | | AMREWFSDGQKLLFARGTMLKVDL | |
| | CDR1-a | TSDQSYG | 403 |
| | CDR2-a | QGSYDEQN | 404 |
| | CDR3-a | CAMREWFSDGQKLLF | 825 |
| | FWR1-a | AQKITQTQPGMFVQEKEAVTLDCTYD | 406 |
| | FWR2-a | LFWYKQPSSGEMIFLIY | 407 |
| | FWR3-a | ATEGRYSLNFQKARKSANLVISASQLGDSAMYF | 408 |
| | FWR4-a | ARGTMLKVDL | 392 |
| TCRB- | b-Variable | GATGCTGATGTTACCCAGACCCCAAGGAATAGGATCACAAAGAC | 826 |
| 79 | nucleotide | AGGAAAGAGGATTATGCTGGAATGTTCTCAGACTAAGGGTCATG | |
| | | ATAGAATGTACTGGTATCGACAAGACCCAGGACTGGGCCTACGG | |
| | | TTGATCTATTACTCCTTTGATGTCAAAGATATAAACAAAGGAGAG | |
| | | ATCTCTGATGGATACAGTGTCTCTCGACAGGCACAGGCTAAATTC | |
| | | TCCCTGTCCCTAGAGTCTGCCATCCCCAACCAGACAGCTCTTTACT | |
| | | TCTGTGCCACCAGTGATGGTCAAGGGAAGCAGCCCCAGCATTTTG | |
| | | GTGATGGGACTCGACTCTCCATCCTAG | |
| | Variable-b | DADVTQTPRNRITKTGKRIMLECSQTKGHDRMYWYRQDPGLGLRLI | 827 |
| | | YYSFDVKDINKGEISDGYSVSRQAQAKFSLSLESAIPNQTALYFCATS | |
| | | DGQGKQPQHFGDGTRLSIL | |
| | CDR1-b | KGHDR | 21 |
| | CDR2-b | SFDVKD | 22 |
| | CDR3-b | CATSDGQGKQPQHF | 828 |
| | FWR1-b | DADVTQTPRNRITKTGKRIMLECSQT | 24 |
| | FWR2-b | MYWYRQDPGLGLRLIYY | 25 |
| | FWR3-b | INKGEISDGYSVSRQAQAKFSLSLESAIPNQTALYF | 26 |
| | FWR4-b | GDGTRLSIL | 27 |
| TCRA- | a-Variable | AGCCAAAAGATAGAACAGAATTCCGAGGCCCTGAACATTCAGGA | 829 |
| 79 | nucleotide | GGGTAAAACGGCCACCCTGACCTGCAACTATACAAACTATTCCCC | |
| | | AGCATACTTACAGTGGTACCGACAAGATCCAGGAAGAGGCCCTG | |
| | | TTTTCTTGCTACTCATACGTGAAAATGAGAAAGAAAAAAGGAAA | |
| | | GAAAGACTGAAGGTCACCTTTGATACCACCCTTAAACAGAGTTTG | |
| | | TTTCATATCACAGCCTCCCAGCCTGCAGACTCAGCTACCTACCTCT | |
| | | GTGCTCTAGAGGGTAATGCAGGCAACATGCTCACCTTTGGAGGG | |
| | | GGAACAAGGTTAATGGTCAAACCCC | |
| | Variable-a | SQKIEQNSEALNIQEGKTATLTCNYTNYSPAYLQWYRQDPGRGPVFL | 830 |
| | | LLIRENEKEKRKERLKVTFDTTLKQSLFHITASQPADSATYLCALEGN | |
| | | AGNMLTFGGGTRLMVKP | |
| | CDR1-a | NYSPAY | 831 |
| | CDR2-a | IRENEKE | 832 |
| | CDR3-a | CALEGNAGNMLTF | 833 |
| | FWR1-a | SQKIEQNSEALNIQEGKTATLTCNYT | 834 |
| | FWR2-a | LQWYRQDPGRGPVFLLL | 835 |
| | FWR3-a | KRKERLKVTFDTTLKQSLFHITASQPADSATYL | 836 |
| | FWR4-a | GGGTRLMVKP | 141 |
| TCRB- | b-Variable | GATTCTGGAGTCACACAAACCCCAAAGCACCTGATCACAGCAAC | 837 |
| 80 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCGTAGGGGGGGCGCGACAGCCTCGTGGCTACA | |
| | | CCTTCGGTTCGGGGACCAGGTTAACCGTTGTAG | |
| | Variable-b | DSGVTQTPKHLITATGQRVTLRCSPRSGDLSVYWYQQSLDQGLQFLI | 838 |
| | | QYYNGEERAKGNILERFSAQQFPDLHSELNLSSLELGDSALYFCASS | |
| | | VGGARQPRGYTFGSGTRLTVV | |
| | CDR1-b | SGDLS | 383 |
| | CDR2-b | YYNGEE | 384 |
| | CDR3-b | CASSVGGARQPRGYTF | 839 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GSGTRLTVV | 103 |
| TCRA- | a-Variable | ATACTGAACGTGGAACAAAGTCCTCAGTCACTGCATGTTCAGGAG | 840 |
| 80 | nucleotide | GGAGACAGCACCAATTTCACCTGCAGCTTCCCTTCCAGCAATTTT | |
| | | TATGCCTTACACTGGTACAGATGGGAAACTGCAAAAAGCCCCGA | |
| | | GGCCTTGTTTGTAATGACTTTAAATGGGGATGAAAAGAAGAAAG | |
| | | GACGAATAAGTGCCACTCTTAATACCAAGGAGGGTTACAGCTATT | |
| | | TGTACATCAAAGGATCCCAGCCTGAAGACTCAGCCACATACCTCT | |
| | | GTGCCTTAATTTCAGGAAACACACCTCTTGTCTTTGGAAAGGGCA | |
| | | CAAGACTTTCTGTGATTGCAA | |
| | Variable-a | ILNVEQSPQSLHVQEGDSTNFTCSFPSSNFYALHWYRWETAKSPEAL | 841 |
| | | FVMTLNGDEKKKGRISATLNTKEGYSYLYIKGSQPEDSATYLCALIS | |
| | | GNTPLVFGKGTRLSVIA | |
| | CDR1-a | SSNFYA | 762 |
| | CDR2-a | MTLNGDE | 763 |
| | CDR3-a | CALISGNTPLVF | 842 |
| | FWR1-a | ILNVEQSPQSLHVQEGDSTNFTCSFP | 765 |
| | FWR2-a | LHWYRWETAKSPEALFV | 766 |
| | FWR3-a | KKKGRISATLNTKEGYSYLYIKGSQPEDSATYL | 767 |
| | FWR4-a | GKGTRLSVIA | 546 |
| TCRB- | b-Variable | GAGGCTGGAGTCACACAAAGTCCCACACACCTGATCAAAACGAG | 843 |
| 81 | nucleotide | AGGACAGCAAGCGACTCTGAGATGCTCTCCTATCTCTGGGCACAC | |
| | | CAGTGTGTACTGGTACCAACAGGCCCTGGGTCTGGGCCTCCAGTT | |
| | | CCTCCTTTGGTATGACGAGGGTGAAGAGAGAAACAGAGGAAACT | |
| | | TCCCTCCTAGATTTTCAGGTCGCCAGTTCCCTAATTATAGCTCTGA | |
| | | GCTGAATGTGAACGCCTTGGAGCTGGAGGACTCGGCCCTGTATCT | |
| | | CTGTGCCAGCAGCCAGACCGGGACAGGGGGCACGTTCACCGGGG | |
| | | AGCTGTTTTTTGGAGAAGGCTCTAGGCTGACCGTACTGG | |
| | Variable-b | EAGVTQSPTHLIKTRGQQATLRCSPISGHTSVYWYQQALGLGLQFLL | 844 |
| | | WYDEGEERNRGNFPPRFSGRQFPNYSSELNVNALELEDSALYLCASS | |
| | | QTGTGGTFTGELFFGEGSRLTVL | |
| | CDR1-b | SGHTS | 845 |
| | CDR2-b | YDEGEE | 846 |
| | CDR3-b | CASSQTGTGGTFTGELFF | 847 |
| | FWR1-b | EAGVTQSPTHLIKTRGQQATLRCSPI | 848 |
| | FWR2-b | VYWYQQALGLGLQFLLW | 849 |
| | FWR3-b | RNRGNFPPRFSGRQFPNYSSELNVNALELEDSALYL | 850 |
| | FWR4-b | GEGSRLTVL | 63 |
| TCRA- | a-Variable | CAACAACCAGTGCAGAGTCCTCAAGCCGTGATCCTCCGAGAAGG | 851 |
| 81 | nucleotide | GGAAGATGCTGTCATCAACTGCAGTTCCTCCAAGGCTTTATATTC | |
| | | TGTACACTGGTACAGGCAGAAGCATGGTGAAGCACCCGTCTTCCT | |
| | | GATGATATTACTGAAGGGTGGAGAACAGAAGGGTCATGAAAAAA | |
| | | TATCTGCTTCATTTAATGAAAAAAAGCAGCAAAGCTCCCTGTACC | |
| | | TTACGGCCTCCCAGCTCAGTTACTCAGGAACCTACTTCTGCGGCA | |
| | | CACCCAGATTTAACACCGACAAGCTCATCTTTGGGACTGGGACCA | |
| | | GATTACAAGTCTTTCCAA | |
| | Variable-a | QQPVQSPQA VILREGEDA VINCSSSKALYSVHWYRQKHGEAPVFLMI | 852 |
| | | LLKGGEQKGHEKISASFNEKKQQSSLYLTASQLSYSGTYFCGTPRFN | |
| | | TDKLIFGTGTRLQVFP | |
| | CDR1-a | KALYS | 30 |
| | CDR2-a | LLKGGEQ | 31 |
| | CDR3-a | CGTPRFNTDKLIF | 853 |
| | FWR1-a | QQPVQSPQA VILREGEDA VINCSSS | 33 |
| | FWR2-a | VHWYRQKHGEAPVFLMI | 854 |
| | FWR3-a | KGHEKISASFNEKKQQSSLYLTASQLSYSGTYF | 855 |
| | FWR4-a | GTGTRLQVFP | 36 |
| TCRB- | b-Variable | GAAGCTGACATCTACCAGACCCCAAGATACCTTGTTATAGGGACA | 856 |
| 82 | nucleotide | GGAAAGAAGATCACTCTGGAATGTTCTCAAACCATGGGCCATGA | |
| | | CAAAATGTACTGGTATCAACAAGATCCAGGAATGGAACTACACC | |
| | | TCATCCACTATTCCTATGGAGTTAATTCCACAGAGAAGGGAGATC | |
| | | TTTCCTCTGAGTCAACAGTCTCCAGAATAAGGACGGAGCATTTTC | |
| | | CCCTGACCCTGGAGTCTGCCAGGCCCTCACATACCTCTCAGTACC | |
| | | TCTGTGCCAGCAGTGGTTCCTATAGCAATCAGCCCCAGCATTTTG | |
| | | GTGATGGGACTCGACTCTCCATCCTAG | |
| | Variable-b | EADIYQTPRYL VIGTGKKITLECSQTMGHDKMYWYQQDPGMELHLI | 857 |
| | | HYSYGVNSTEKGDLSSESTVSRIRTEHFPLTLESARPSHTSQYLCASS | |
| | | GSYSNQPQHFGDGTRLSIL | |
| | CDR1-b | MGHDK | 858 |
| | CDR2-b | SYGVNS | 859 |
| | CDR3-b | CASSGSYSNQPQHF | 860 |
| | FWR1-b | EADIYQTPRYL VIGTGKKITLECSQT | 861 |
| | FWR2-b | MYWYQQDPGMELHLIHY | 862 |
| | FWR3-b | TEKGDLSSESTVSRIRTEHFPLTLESARPSHTSQYL | 863 |
| | FWR4-b | GDGTRLSIL | 27 |
| TCRA- | a-Variable | CAGAAGGAGGTGGAGCAGGATCCTGGACCACTCAGTGTTCCAGA | 864 |
| 82 | nucleotide | GGGAGCCATTGTTTCTCTCAACTGCACTTACAGCAACAGTGCTTT | |
| | | TCAATACTTCATGTGGTACAGACAGTATTCCAGAAAAGGCCCTGA | |
| | | GTTGCTGATGTACACATACTCCAGTGGTAACAAAGAAGATGGAA | |
| | | GGTTTACAGCACAGGTCGATAAATCCAGCAAGTATATCTCCTTGT | |
| | | TCATCAGAGACTCACAGCCCAGTGATTCAGCCACCTACCTCTGTG | |
| | | CAATCAAAATTCAGGGAGCCCAGAAGCTGGTATTTGGCCAAGGA | |
| | | ACCAGGCTGACTATCAACCCAA | |
| | Variable-a | QKEVEQDPGPLSVPEGAIVSLNCTYSNSAFQYFMWYRQYSRKGPEL | 865 |
| | | LMYTYSSGNKEDGRFTAQVDKSSKYISLFIRDSQPSDSATYLCAIKIQ | |
| | | GAQKLVFGQGTRLTINP | |
| | CDR1-a | NSAFQY | 673 |
| | CDR2-a | TYSSGN | 674 |
| | CDR3-a | CAIKIQGAQKLVF | 866 |
| | FWR1-a | QKEVEQDPGPLSVPEGAIVSLNCTYS | 676 |
| | FWR2-a | FMWYRQYSRKGPELLMY | 677 |
| | FWR3-a | KEDGRFTAQVDKSSKYISLFIRDSQPSDSATYL | 678 |
| | FWR4-a | GQGTRLTINP | 867 |
| TCRB- | b-Variable | GACACAGCTGTTTCCCAGACTCCAAAATACCTGGTCACACAGATG | 868 |
| 83 | nucleotide | GGAAACGACAAGTCCATTAAATGTGAACAAAATCTGGGCCATGA | |
| | | TACTATGTATTGGTATAAACAGGACTCTAAGAAATTTCTGAAGAT | |
| | | AATGTTTAGCTACAATAATAAGGAGCTCATTATAAATGAAACAGT | |
| | | TCCAAATCGCTTCTCACCTAAATCTCCAGACAAAGCTCACTTAAA | |
| | | TCTTCACATCAATTCCCTGGAGCTTGGTGACTCTGCTGTGTATTTC | |
| | | TGTGCCAGCAGCCAAGAAGACAGGTCTGGAAACACCATATATTTT | |
| | | GGAGAGGGAAGTTGGCTCACTGTTGTAG | |
| | Variable-b | DTAVSQTPKYLVTQMGNDKSIKCEQNLGHDTMYWYKQDSKKFLKI | 869 |
| | | MFSYNNKELIINETVPNRFSPKSPDKAHLNLHINSLELGDSAVYFCAS | |
| | | SQEDRSGNTIYFGEGSWLTVV | |
| | CDR1-b | LGHDT | 342 |
| | CDR2-b | YNNKEL | 343 |
| | CDR3-b | CASSQEDRSGNTIYF | 870 |
| | FWR1-b | DTAVSQTPKYLVTQMGNDKSIKCEQN | 345 |
| | FWR2-b | MYWYKQDSKKFLKIMFS | 346 |
| | FWR3-b | IINETVPNRFSPKSPDKAHLNLHINSLELGDSAVYF | 347 |
| | FWR4-b | GEGSWLTVV | 871 |
| TCRA- | a-Variable | GGAGAGGATGTGGAGCAGAGTCTTTTCCTGAGTGTCCGAGAGGG | 872 |
| 83 | nucleotide | AGACAGCTCCGTTATAAACTGCACTTACACAGACAGCTCCTCCAC | |
| | | CTACTTATACTGGTATAAGCAAGAACCTGGAGCAGGTCTCCAGTT | |
| | | GCTGACGTATATTTTTTCAAATATGGACATGAAACAAGACCAAAG | |
| | | ACTCACTGTTCTATTGAATAAAAAGGATAAACATCTGTCTCTGCG | |
| | | CATTGCAGACACCCAGACTGGGGACTCAGCTATCTACTTCTGTGC | |
| | | AGAGATCCTTTTAAGGGGAGGCTTCAAAACTATCTTTGGAGCAGG | |
| | | AACAAGACTATTTGTTAAAGCAA | |
| | Variable-a | GEDVEQSLFLSVREGDSSVINCTYTDSSSTYLYWYKQEPGAGLQLLT | 873 |
| | | YIFSNMDMKQDQRLTVLLNKKDKHLSLRIADTQTGDSAIYFCAEILL | |
| | | RGGFKTIFGAGTRLFVKA | |
| | CDR1-a | DSSSTY | 314 |
| | CDR2-a | IFSNMDM | 315 |
| | CDR3-a | CAEILLRGGFKTIF | 874 |
| | FWR1-a | GEDVEQSLFLSVREGDSSVINCTYT | 317 |
| | FWR2-a | LYWYKQEPGAGLQLLTY | 318 |
| | FWR3-a | KQDQRLTVLLNKKDKHLSLRIADTQTGDSAIYF | 319 |
| | FWR4-a | GAGTRLFVKA | 112 |
| TCRB- | b-Variable | GATGGTGGAATCACTCAGTCCCCAAAGTACCTGTTCAGAAAGGA | 875 |
| 84 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGTATCATCTCAGGTTCCTACGAGCAGTACTTC | |
| | | GGGCCGGGCACCAGGCTCACGGTCACAG | |
| | Variable-b | DGGITQSPKYLFRKEGQNVTLSCEQNLNHDAMYWYRQDPGQGLRLI | 876 |
| | | YYSQIVNDFQKGDIAEGYSVSREKKESFPLTVTSAQKNPTAFYLCAS | |
| | | SIISGSYEQYFGPGTRLTVT | |
| | CDR1-b | LNHDA | 216 |
| | CDR2-b | SQIVND | 217 |
| | CDR3-b | CASSIISGSYEQYF | 877 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVT | 85 |
| TCRA- | a-Variable | GACCAGCAAGTTAAGCAAAATTCACCATCCCTGAGCGTCCAGGA | 878 |
| 84 | nucleotide | AGGAAGAATTTCTATTCTGAACTGTGACTATACTAACAGCATGTT | |
| | | TGATTATTTCCTATGGTACAAAAAATACCCTGCTGAAGGTCCTAC | |
| | | ATTCCTGATATCTATAAGTTCCATTAAGGATAAAAATGAAGATGG | |
| | | AAGATTCACTGTCTTCTTAAACAAAAGTGCCAAGCACCTCTCTCT | |
| | | GCACATTGTGCCCTCCCAGCCTGGAGACTCTGCAGTGTACTTCTG | |
| | | TGCAGCAAGCGCGGTGAGCAACTATAAACTGACATTTGGAAAAG | |
| | | GAACTCTCTTAACCGTGAATCCAA | |
| | Variable-a | DQQVKQNSPSLSVQEGRISILNCDYTNSMFDYFLWYKKYPAEGPTFL | 879 |
| | | ISISSIKDKNEDGRFTVFLNKSAKHLSLHIVPSQPGDSAVYFCAASAVS | |
| | | NYKLTFGKGTLLTVNP | |
| | CDR1-a | NSMFDY | 303 |
| | CDR2-a | ISSIKDK | 304 |
| | CDR3-a | CAASAVSNYKLTF | 880 |
| | FWR1-a | DQQVKQNSPSLSVQEGRISILNCDYT | 306 |
| | FWR2-a | FLWYKKYPAEGPTFLIS | 307 |
| | FWR3-a | NEDGRFTVFLNKSAKHLSLHIVPSQPGDSAVYF | 308 |
| | FWR4-a | GKGTLLTVNP | 662 |
| TCRB- | b-Variable | GATGGTGGAATCACTCAGTCCCCAAAGTACCTGTTCAGAAAGGA | 881 |
| 85 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGCGGGAGGGCCACGTCCTACAATGAGCAGTT | |
| | | CTTCGGGCCAGGGACACGGCTCACCGTGCTAG | |
| | Variable-b | DGGITQSPKYLFRKEGQNVTLSCEQNLNHDAMYWYRQDPGQGLRLI | 882 |
| | | YYSQIVNDFQKGDIAEGYSVSREKKESFPLTVTSAQKNPTAFYLCAS | |
| | | SGRATSYNEQFFGPGTRLTVL | |
| | CDR1-b | LNHDA | 216 |
| | CDR2-b | SQIVND | 217 |
| | CDR3-b | CASSGRATSYNEQFF | 883 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVL | 9 |
| TCRA- | a-Variable | GCCCAGTCAGTGACCCAGCCTGACATCCACATCACTGTCTCTGAA | 884 |
| 85 | nucleotide | GGAGCCTCACTGGAGTTGAGATGTAACTATTCCTATGGGGCAACA | |
| | | CCTTATCTCTTCTGGTATGTCCAGTCCCCCGGCCAAGGCCTCCAGC | |
| | | TGCTCCTGAAGTACTTTTCAGGAGACACTCTGGTTCAAGGCATTA | |
| | | AAGGCTTTGAGGCTGAATTTAAGAGGAGTCAATCTTCCTTCAATC | |
| | | TGAGGAAACCCTCTGTGCATTGGAGTGATGCTGCTGAGTACTTCT | |
| | | GTGCTGTGGGGGACGGTGGTGCTACAAACAAGCTCATCTTTGGAA | |
| | | CTGGCACTCTGCTTGCTGTCCAGCCAA | |
| | Variable-a | AQSVTQPDIHITVSEGASLELRCNYSYGATPYLFWYVQSPGQGLQLL | 885 |
| | | LKYFSGDTLVQGIKGFEAEFKRSQSSFNLRKPSVHWSDAAEYFCAVG | |
| | | DGGATNKLIFGTGTLLAVQP | |
| | CDR1-a | YGATPY | 886 |
| | CDR2-a | YFSGDTLV | 887 |
| | CDR3-a | CAVGDGGATNKLIF | 888 |
| | FWR1-a | AQSVTQPDIHITVSEGASLELRCNYS | 889 |
| | FWR2-a | LFWYVQSPGQGLQLLLK | 890 |
| | FWR3-a | QGIKGFEAEFKRSQSSFNLRKPSVHWSDAAEYF | 891 |
| | FWR4-a | GTGTLLAVQP | 480 |
| TCRB- | b-Variable | GAAGCTGGAGTTGCCCAGTCTCCCAGATATAAGATTATAGAGAA | 892 |
| 86 | nucleotide | AAGGCAGAGTGTGGCTTTTTGGTGCAATCCTATATCTGGCCATGC | |
| | | TACCCTTTACTGGTACCAGCAGATCCTGGGACAGGGCCCAAAGCT | |
| | | TCTGATTCAGTTTCAGAATAACGGTGTAGTGGATGATTCACAGTT | |
| | | GCCTAAGGATCGATTTTCTGCAGAGAGGCTCAAAGGAGTAGACT | |
| | | CCACTCTCAAGATCCAGCCTGCAAAGCTTGAGGACTCGGCCGTGT | |
| | | ATCTCTGTGCCAGCAGCCCCCTCGATCCCTACGAGCAGTACTTCG | |
| | | GGCCGGGCACCAGGCTCACGGTCACAG | |
| | Variable-b | EAGVAQSPRYKIIEKRQSVAFWCNPISGHATLYWYQQILGQGPKLLI | 893 |
| | | QFQNNGVVDDSQLPKDRFSAERLKGVDSTLKIQPAKLEDSAVYLCA | |
| | | SSPLDPYEQYFGPGTRLTVT | |
| | CDR1-b | SGHAT | 894 |
| | CDR2-b | FQNNGV | 895 |
| | CDR3-b | CASSPLDPYEQYF | 896 |
| | FWR1-b | EAGVAQSPRYKIIEKRQSVAFWCNPI | 897 |
| | FWR2-b | LYWYQQILGQGPKLLIQ | 898 |
| | FWR3-b | VDDSQLPKDRFSAERLKGVDSTLKIQPAKLEDSAVYL | 899 |
| | FWR4-b | GPGTRLTVT | 85 |
| TCRA- | a-Variable | GGAGAGAATGTGGAGCAGCATCCTTCAACCCTGAGTGTCCAGGA | 900 |
| 86 | nucleotide | GGGAGACAGCGCTGTTATCAAGTGTACTTATTCAGACAGTGCCTC | |
| | | AAACTACTTCCCTTGGTATAAGCAAGAACTTGGAAAAAGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCAGGGTCTAAATCCGGGGGTTACCAGAAAGTTACCTTTG | |
| | | GAACTGGAACAAAGCTCCAAGTCATCCCAA | |
| | Variable-a | GENVEQHPSTLSVQEGDSAVIKCTYSDSASNYFPWYKQELGKRPQLI | 901 |
| | | IDIRSNVGEKKDQRIA VTLNKTAKHFSLHITETQPEDSAVYFCAAGSK | |
| | | SGGYQKVTFGTGTKLQVIP | |
| | CDR1-a | DSASNY | 207 |
| | CDR2-a | IRSNVGE | 208 |
| | CDR3-a | CAAGSKSGGYQKVTF | 902 |
| | FWR1-a | GENVEQHPSTLSVQEGDSAVIKCTYS | 210 |
| | FWR2-a | FPWYKQELGKRPQLIID | 211 |
| | FWR3-a | KKDQRIAVTLNKTAKHFSLHITETQPEDSAVYF | 212 |
| | FWR4-a | GTGTKLQVIP | 903 |
| TCRB- | b-Variable | GATGGTGGAATCACTCAGTCCCCAAAGTACCTGTTCAGAAAGGA | 904 |
| 87 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTACCCCTAGGGCTAGCGGGGAGATCGCCGATGA | |
| | | GCAGTTCTTCGGGCCAGGGACACGGCTCACCGTGCTAG | |
| | Variable-b | DGGITQSPKYLFRKEGQNVTLSCEQNLNHDAMYWYRQDPGQGLRLI | 905 |
| | | YYSQIVNDFQKGDIAEGYSVSREKKESFPLTVTSAQKNPTAFYLCAS | |
| | | TPRASGEIADEQFFGPGTRLTVL | |
| | CDR1-b | LNHDA | 216 |
| | CDR2-b | SQIVND | 217 |
| | CDR3-b | CASTPRASGEIADEQFF | 906 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVL | 9 |
| TCRA- | a-Variable | GCTCAGAAGGTAACTCAAGCGCAGACTGAAATTTCTGTGGTGGA | 907 |
| 87 | nucleotide | GAAGGAGGATGTGACCTTGGACTGTGTGTATGAAACCCGTGATA | |
| | | CTACTTATTACTTATTCTGGTACAAGCAACCACCAAGTGGAGAAT | |
| | | TGGTTTTCCTTATTCGTCGGAACTCTTTTGATGAGCAAAATGAAAT | |
| | | AAGTGGTCGGTATTCTTGGAACTTCCAGAAATCCACCAGTTCCTT | |
| | | CAACTTCACCATCACAGCCTCACAAGTCGTGGACTCAGCAGTATA | |
| | | CTTCTGTGCTCTGAGTGAGGGGGGATACAACTTCAACAAATTTTA | |
| | | CTTTGGATCTGGGACCAAACTCAATGTAAAACCAA | |
| | Variable-a | AQKVTQAQTEISVVEKEDVTLDCVYETRDTTYYLFWYKQPPSGELV | 908 |
| | | FLIRRNSFDEQNEISGRYSWNFQKSTSSFNFTITASQVVDSAVYFCAL | |
| | | SEGGYNFNKFYFGSGTKLNVKP | |
| | CDR1-a | TRDTTYY | 48 |
| | CDR2-a | RNSFDEQN | 49 |
| | CDR3-a | CALSEGGYNFNKFYF | 909 |
| | FWR1-a | AQKVTQAQTEISVVEKEDVTLDCVYE | 51 |
| | FWR2-a | LFWYKQPPSGEL VFLIR | 52 |
| | FWR3-a | EISGRYSWNFQKSTSSFNFTITASQVVDSAVYF | 53 |
| | FWR4-a | GSGTKLNVKP | 910 |
| TCRB- | b-Variable | GAAGCTGGAGTTACTCAGTTCCCCAGCCACAGCGTAATAGAGAA | 911 |
| 88 | nucleotide | GGGCCAGACTGTGACTCTGAGATGTGACCCAATTTCTGGACATGA | |
| | | TAATCTTTATTGGTATCGACGTGTTATGGGAAAAGAAATAAAATT | |
| | | TCTGTTACATTTTGTGAAAGAGTCTAAACAGGATGAGTCCGGTAT | |
| | | GCCCAACAATCGATTCTTAGCTGAAAGGACTGGAGGGACGTATTC | |
| | | TACTCTGAAGGTGCAGCCTGCAGAACTGGAGGATTCTGGAGTTTA | |
| | | TTTCTGTGCCAGCAGCCAAGAAGTCAGCCGCCCACACAATGAGC | |
| | | AGTTCTTCGGGCCAGGGACACGGCTCACCGTGCTAG | |
| | Variable-b | EAGVTQFPSHSVIEKGQTVTLRCDPISGHDNLYWYRRVMGKEIKFLL | 912 |
| | | HFVKESKQDESGMPNNRFLAERTGGTYSTLKVQPAELEDSGVYFCA | |
| | | SSQEVSRPHNEQFFGPGTRLTVL | |
| | CDR1-b | SGHDN | 57 |
| | CDR2-b | FVKESK | 58 |
| | CDR3-b | CASSQEVSRPHNEQFF | 913 |
| | FWR1-b | EAGVTQFPSHSVIEKGQTVTLRCDPI | 60 |
| | FWR2-b | LYWYRRVMGKEIKFLLH | 61 |
| | FWR3-b | QDESGMPNNRFLAERTGGTYSTLKVQPAELEDSGVYF | 62 |
| | FWR4-b | GPGTRLTVL | 9 |
| TCRA- | a-Variable | GGAATACAAGTGGAGCAGAGTCCTCCAGACCTGATTCTCCAGGA | 914 |
| 88 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTGGA | |
| | | GCCTCTCACGGGAGGAGGAAACAAACTCACCTTTGGGACAGGCA | |
| | | CTCAGCTAAAAGTGGAACTCA | |
| | Variable-a | GIQVEQSPPDLILQEGANSTLRCNFSDSVNNLQWFHQNPWGQLINLF | 915 |
| | | YIPSGTKQNGRLSATTVATERYSLLYISSSQTTDSGVYFCAVEPLTGG | |
| | | GNKLTFGTGTQLKVEL | |
| | CDR1-a | DSVNN | 506 |
| | CDR2-a | IPSGT | 507 |
| | CDR3-a | CAVEPLTGGGNKLTF | 916 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GTGTQLKVEL | 456 |
| TCRB- | b-Variable | GATGTGAAAGTAACCCAGAGCTCGAGATATCTAGTCAAAAGGAC | 917 |
| 89 | nucleotide | GGGAGAGAAAGTTTTTCTGGAATGTGTCCAGGATATGGACCATG | |
| | | AAAATATGTTCTGGTATCGACAAGACCCAGGTCTGGGGCTACGGC | |
| | | TGATCTATTTCTCATATGATGTTAAAATGAAAGAAAAAGGAGATA | |
| | | TTCCTGAGGGGTACAGTGTCTCTAGAGAGAAGAAGGAGCGCTTCT | |
| | | CCCTGATTCTGGAGTCCGCCAGCACCAACCAGACATCTATGTACC | |
| | | TCTGTGCCAGCAGTTTATTACCCAGAGGGGGTGGTGGTGGCTACA | |
| | | CCTTCGGTTCGGGGACCAGGTTAACCGTTGTAG | |
| | Variable-b | DVKVTQSSRYLVKRTGEKVFLECVQDMDHENMFWYRQDPGLGLRL | 918 |
| | | IYFSYDVKMKEKGDIPEGYSVSREKKERFSLILESASTNQTSMYLCAS | |
| | | SLLPRGGGGGYTFGSGTRLTVV | |
| | CDR1-b | MDHEN | 199 |
| | CDR2-b | SYDVKM | 200 |
| | CDR3-b | CASSLLPRGGGGGYTF | 919 |
| | FWR1-b | DVKVTQSSRYLVKRTGEKVFLECVQD | 202 |
| | FWR2-b | MFWYRQDPGLGLRLIYF | 203 |
| | FWR3-b | KEKGDIPEGYSVSREKKERFSLILESASTNQTSMYL | 204 |
| | FWR4-b | GSGTRLTVV | 103 |
| TCRA- | a-Variable | AAGGACCAAGTGTTTCAGCCTTCCACAGTGGCATCTTCAGAGGGA | 920 |
| 89 | nucleotide | GCTGTGGTGGAAATCTTCTGTAATCACTCTGTGTCCAATGCTTAC | |
| | | AACTTCTTCTGGTACCTTCACTTCCCGGGATGTGCACCAAGACTC | |
| | | CTTGTTAAAGGCTCAAAGCCTTCTCAGCAGGGACGATACAACATG | |
| | | ACCTATGAACGGTTCTCTTCATCGCTGCTCATCCTCCAGGTGCGG | |
| | | GAGGCAGATGCTGCTGTTTACTACTGTGCTGTTGCAGATTCAGGA | |
| | | AACACACCTCTTGTCTTTGGAAAGGGCACAAGACTTTCTGTGATT | |
| | | GCAA | |
| | Variable-a | KDQVFQPSTVASSEGAVVEIFCNHSVSNAYNFFWYLHFPGCAPRLLV | 921 |
| | | KGSKPSQQGRYNMTYERFSSSLLILQVREADAAVYYCAVADSGNTP | |
| | | LVFGKGTRLSVIA | |
| | CDR1-a | VSNAYN | 236 |
| | CDR2-a | GSKP | 237 |
| | CDR3-a | CAVADSGNTPLVF | 922 |
| | FWR1-a | KDQVFQPSTVASSEGAVVEIFCNHS | 239 |
| | FWR2-a | FFWYLHFPGCAPRLLVK | 240 |
| | FWR3-a | SQQGRYNMTYERFSSSLLILQVREADAAVYY | 241 |
| | FWR4-a | GKGTRLSVIA | 546 |
| TCRB- | b-Variable | AATGCTGGTGTCACTCAGACCCCAAAATTCCAGGTCCTGAAGACA | 923 |
| 90 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTTACGCTAGCCTCACGGACCAGTTCTTCGGGCC | |
| | | AGGGACACGGCTCACCGTGCTAG | |
| | Variable-b | NAGVTQTPKFQVLKTGQSMTLQCAQDMNHEYMSWYRQDPGMGLR | 924 |
| | | LIHYSVGAGITDQGEVPNGYNVSRSTTEDFPLRLLSAAPSQTSVYFCA | |
| | | SSYASLTDQFFGPGTRLTVL | |
| | CDR1-b | MNHEY | 252 |
| | CDR2-b | SVGAGI | 253 |
| | CDR3-b | CASSYASLTDQFF | 925 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GPGTRLTVL | 9 |
| TCRA- | a-Variable | ACCCAGCTGCTGGAGCAGAGCCCTCAGTTTCTAAGCATCCAAGAG | 926 |
| 90 | nucleotide | GGAGAAAATCTCACTGTGTACTGCAACTCCTCAAGTGTTTTTTCC | |
| | | AGCTTACAATGGTACAGACAGGAGCCTGGGGAAGGTCCTGTCCT | |
| | | CCTGGTGACAGTAGTTACGGGTGGAGAAGTGAAGAAGCTGAAGA | |
| | | GACTAACCTTTCAGTTTGGTGATGCAAGAAAGGACAGTTCTCTCC | |
| | | ACATCACTGCGGCCCAGCCTGGTGATACAGGCCTCTACCTCTGTG | |
| | | CAGGAGCTGAGGATGCCAGACTCATGTTTGGAGATGGAACTCAG | |
| | | CTGGTGGTGAAGCCCA | |
| | Variable-a | TQLLEQSPQFLSIQEGENLTVYCNSSSVFSSLQWYRQEPGEGPVLLVT | 927 |
| | | VVTGGEVKKLKRLTFQFGDARKDSSLHITAAQPGDTGLYLCAGAED | |
| | | ARLMFGDGTQLVVKP | |
| | CDR1-a | SVFSS | 533 |
| | CDR2-a | VVTGGEV | 534 |
| | CDR3-a | CAGAEDARLMF | 928 |
| | FWR1-a | TQLLEQSPQFLSIQEGENLTVYCNSS | 536 |
| | FWR2-a | LQWYRQEPGEGPVLLVT | 537 |
| | FWR3-a | KKLKRLTFQFGDARKDSSLHITAAQPGDTGLYL | 538 |
| | FWR4-a | GDGTQLVVKP | 230 |
| TCRB- | b-Variable | GATACTGGAGTCTCCCAGGACCCCAGACACAAGATCACAAAGAG | 929 |
| 91.1 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCTTGAGTCGGGGACAGGGGGCTTTTCAGC | |
| | | CCCAGCATTTTGGTGATGGGACTCGACTCTCCATCCTAG | |
| | Variable-b | DTGVSQDPRHKITKRGQNVTFRCDPISEHNRLYWYRQTLGQGPEFLT | 930 |
| | | YFQNEAQLEKSRLLSDRFSAERPKGSFSTLEIQRTEQGDSAMYLCASS | |
| | | LSRGQGAFQPQHFGDGTRLSIL | |
| | CDR1-b | SEHNR | 442 |
| | CDR2-b | FQNEAQ | 443 |
| | CDR3-b | CASSLSRGQGAFQPQHF | 931 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GDGTRLSIL | 27 |
| TCRB- | b-Variable | GACACTGAAGTTACCCAGACACCAAAACACCTGGTCATGGGAAT | 932 |
| 91.2 | nucleotide | GACAAATAAGAAGTCTTTGAAATGTGAACAACATATGGGGCACA | |
| | | GGGCTATGTATTGGTACAAGCAGAAAGCTAAGAAGCCACCGGAG | |
| | | CTCATGTTTGTCTACAGCTATGAGAAACTCTCTATAAATGAAAGT | |
| | | GTGCCAAGTCGCTTCTCACCTGAATGCCCCAACAGCTCTCTCTTA | |
| | | AACCTTCACCTACACGCCCTGCAGCCAGAAGACTCAGCCCTGTAT | |
| | | CTCTGCGCCAGCAGCCAGGCAACTAATGAAAAACTGTTTTTTGGC | |
| | | AGTGGAACCCAGCTCTCTGTCTTGG | |
| | Variable-b | DTEVTQTPKHLVMGMTNKKSLKCEQHMGHRAMYWYKQKAKKPP | 933 |
| | | ELMFVYSYEKLSINESVPSRFSPECPNSSLLNLHLHALQPEDSALYLC | |
| | | ASSQATNEKLFFGSGTQLSVL | |
| | CDR1-b | MGHRA | 639 |
| | CDR2-b | YSYEKL | 640 |
| | CDR3-b | CASSQATNEKLFF | 934 |
| | FWR1-b | DTEVTQTPKHLVMGMTNKKSLKCEQH | 642 |
| | FWR2-b | MYWYKQKAKKPPELMFV | 643 |
| | FWR3-b | SINESVPSRESPECPNSSLLNLHLHALQPEDSALYL | 644 |
| | FWR4-b | GSGTQLSVL | 45 |
| TCRA- | a-Variable | GGAGAGAATGTGGAGCAGCATCCTTCAACCCTGAGTGTCCAGGA | 935 |
| 91 | nucleotide | GGGAGACAGCGCTGTTATCAAGTGTACTTATTCAGACAGTGCCTC | |
| | | AAACTACTTCCCTTGGTATAAGCAAGAACTTGGAAAAGGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCATATGGTACTAGCTATGGAAAGCTGACATTTGGACAA | |
| | | GGGACCATCTTGACTGTCCATCCAA | |
| | Variable-a | GENVEQHPSTLSVQEGDSAVIKCTYSDSASNYFPWYKQELGKGPQLI | 936 |
| | | IDIRSNVGEKKDQRIA VTLNKTAKHFSLHITETQPEDSAVYFCAAYGT | |
| | | SYGKLTFGQGTILTVHP | |
| | CDR1-a | DSASNY | 207 |
| | CDR2-a | IRSNVGE | 208 |
| | CDR3-a | CAAYGTSYGKLTF | 937 |
| | FWR1-a | GENVEQHPSTLSVQEGDSAVIKCTYS | 210 |
| | FWR2-a | FPWYKQELGKGPQLIID | 374 |
| | FWR3-a | KKDQRIAVTLNKTAKHFSLHITETQPEDSAVYF | 212 |
| | FWR4-a | GQGTILTVHP | 76 |
| TCRB- | b-Variable | GGTGCTGTCGTCTCTCAACATCCGAGCTGGGTTATCTGTAAGAGT | 938 |
| 92 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTTGCCGTGACAGGGGCTACACTGAAG | |
| | | CTTTCTTTGGACAAGGCACCAGACTCACAGTTGTAG | |
| | Variable-b | GAVVSQHPSWVICKSGTSVKIECRSLDFQATTMFWYRQFPKQSLML | 939 |
| | | MATSNEGSKATYEQGVEKDKFLINHASLTLSTLTVTSAHPEDSSFYIC | |
| | | SACRDRGYTEAFFGQGTRLTVV | |
| | CDR1-b | DFQATT | 79 |
| | CDR2-b | SNEGSKA | 80 |
| | CDR3-b | CSACRDRGYTEAFF | 940 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GQGTRLTVV | 246 |
| TCRA- | a-Variable | GCTCAGACAGTCACTCAGTCTCAACCAGAGATGTCTGTGCAGGAG | 941 |
| 92 | nucleotide | GCAGAGACCGTGACCCTGAGCTGCACATATGACACCAGTGAGAG | |
| | | TGATTATTATTTATTCTGGTACAAGCAGCCTCCCAGCAGGCAGAT | |
| | | GATTCTCGTTATTCGCCAAGAAGCTTATAAGCAACAGAATGCAAC | |
| | | AGAGAATCGTTTCTCTGTGAACTTCCAGAAAGCAGCCAAATCCTT | |
| | | CAGTCTCAAGATCTCAGACTCACAGCTGGGGGATGCCGCGATGTA | |
| | | TTTCTGTGCTTATAGGTTCAATAACAATGACATGCGCTTTGGAGC | |
| | | AGGGACCAGACTGACAGTAAAACCAA | |
| | Variable-a | AQTVTQSQPEMSVQEAETVTLSCTYDTSESDYYLFWYKQPPSRQMI | 942 |
| | | LVIRQEAYKQQNATENRFSVNFQKAAKSFSLKISDSQLGDAAMYFC | |
| | | AYRFNNNDMRFGAGTRLTVKP | |
| | CDR1-a | TSESDYY | 286 |
| | CDR2-a | QEAYKQQN | 287 |
| | CDR3-a | CAYRFNNNDMRF | 943 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDAAMYF | 291 |
| | FWR4-a | GAGTRLTVKP | 685 |
| TCRB- | b-Variable | TCTCAGACTATTCATCAATGGCCAGCGACCCTGGTGCAGCCTGTG | 944 |
| 93 | nucleotide | GGCAGCCCGCTCTCTCTGGAGTGCACTGTGGAGGGAACATCAAA | |
| | | CCCCAACCTATACTGGTACCGACAGGCTGCAGGCAGGGGCCTCC | |
| | | AGCTGCTCTTCTACTCCGTTGGTATTGGCCAGATCAGCTCTGAGG | |
| | | TGCCCCAGAATCTCTCAGCCTCCAGACCCCAGGACCGGCAGTTCA | |
| | | TCCTGAGTTCTAAGAAGCTCCTTCTCAGTGACTCTGGCTTCTATCT | |
| | | CTGTGCCTGGACCTCCGGACCCAGCACAGATACGCAGTATTTTGG | |
| | | CCCAGGCACCCGGCTGACAGTGCTCG | |
| | Variable-b | SQTIHQWPATLVQPVGSPLSLECTVEGTSNPNLYWYRQAAGRGLQL | 945 |
| | | LFYSVGIGQISSEVPQNLSASRPQDRQFILSSKKLLLSDSGFYLCAWTS | |
| | | GPSTDTQYFGPGTRLTVL | |
| | CDR1-b | GTSNPN | 395 |
| | CDR2-b | SVGIG | 396 |
| | CDR3-b | CAWTSGPSTDTQYF | 946 |
| | FWR1-b | SQTIHQWPATLVQPVGSPLSLECTVE | 398 |
| | FWR2-b | LYWYRQAAGRGLQLLFY | 399 |
| | FWR3-b | QISSEVPQNLSASRPQDRQFILSSKKLLLSDSGFYL | 400 |
| | FWR4-b | GPGTRLTVL | 9 |
| TCRA- | a-Variable | GGAGAGAATGTGGAGCAGCATCCTTCAACCCTGAGTGTCCAGGA | 947 |
| 93 | nucleotide | GGGAGACAGCGCTGTTATCAAGTGTACTTATTCAGACAGTGCCTC | |
| | | AAACTACTTCCCTTGGTATAAGCAAGAACTTGGAAAAGGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCGGCCCCCTCTCGAGGCTTCAAAACTATCTTTGGAGCAGGAA | |
| | | CAAGACTATTTGTTAAAGCAA | |
| | Variable-a | GENVEQHPSTLSVQEGDSAVIKCTYSDSASNYFPWYKQELGKGPQLI | 948 |
| | | IDIRSNVGEKKDQRIA VTLNKTAKHFSLHITETQPEDSAVYFCAAPSR | |
| | | GFKTIFGAGTRLFVKA | |
| | CDR1-a | DSASNY | 207 |
| | CDR2-a | IRSNVGE | 208 |
| | CDR3-a | CAAPSRGFKTIF | 949 |
| | FWR1-a | GENVEQHPSTLSVQEGDSAVIKCTYS | 210 |
| | FWR2-a | FPWYKQELGKGPQLIID | 374 |
| | FWR3-a | KKDQRIAVTLNKTAKHFSLHITETQPEDSAVYF | 212 |
| | FWR4-a | GAGTRLFVKA | 112 |
| TCRB- | b-Variable | GGTGCTGGAGTCTCCCAGTCTCCCAGGTACAAAGTCACAAAGAG | 950 |
| 94 | nucleotide | GGGACAGGATGTAGCTCTCAGGTGTGATCCAATTTCGGGTCATGT | |
| | | ATCCCTTTATTGGTACCGACAGGCCCTGGGGCAGGGCCCAGAGTT | |
| | | TCTGACTTACTTCAATTATGAAGCCCAACAAGACAAATCAGGGCT | |
| | | GCCCAATGATCGGTTCTCTGCAGAGAGGCCTGAGGGATCCATCTC | |
| | | CACTCTGACGATCCAGCGCACAGAGCAGCGGGACTCGGCCATGT | |
| | | ATCGCTGTGCCAGCAGCTCAGGACTCACAGATACGCAGTATTTTG | |
| | | GCCCAGGCACCCGGCTGACAGTGCTCG | |
| | Variable-b | GAGVSQSPRYKVTKRGQDVALRCDPISGHVSLYWYRQALGQGPEFL | 951 |
| | | TYFNYEAQQDKSGLPNDRFSAERPEGSISTLTIQRTEQRDSAMYRCA | |
| | | SSSGLTDTQYFGPGTRLTVL | |
| | CDR1-b | SGHVS | 275 |
| | CDR2-b | FNYEAQ | 276 |
| | CDR3-b | CASSSGLTDTQYF | 952 |
| | FWR1-b | GAGVSQSPRYKVTKRGQDVALRCDPI | 278 |
| | FWR2-b | LYWYRQALGQGPEFLTY | 279 |
| | FWR3-b | QDKSGLPNDRFSAERPEGSISTLTIQRTEQRDSAMYR | 280 |
| | FWR4-b | GPGTRLTVL | 9 |
| TCRA- | a-Variable | AAACAGGAGGTGACACAGATTCCTGCAGCTCTGAGTGTCCCAGA | 953 |
| 94 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGCCCTAGATAACTATGGTCAGAATTTTGTCTTTGGTCCCGG | |
| | | AACCAGATTGTCCGTGCTGCCCT | |
| | Variable-a | KQEVTQIPAALSVPEGENLVLNCSFTDSAIYNLQWFRQDPGKGLTSL | 954 |
| | | LLIQSSQREQTSGRLNASLDKSSGRSTLYIAASQPGDSATYLCAALDN | |
| | | YGQNFVFGPGTRLSVLP | |
| | CDR1-a | DSAIYN | 118 |
| | CDR2-a | IQSSQRE | 119 |
| | CDR3-a | CAALDNYGQNFVF | 955 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GPGTRLSVLP | 272 |
| TCRB- | b-Variable | GACACTGAAGTTACCCAGACACCAAAACACCTGGTCATGGGAAT | 956 |
| 95 | nucleotide | GACAAATAAGAAGTCTTTGAAATGTGAACAACATATGGGGCACA | |
| | | GGGCTATGTATTGGTACAAGCAGAAAGCTAAGAAGCCACCGGAG | |
| | | CTCATGTTTGTCTACAGCTATGAGAAACTCTCTATAAATGAAAGT | |
| | | GTGCCAAGTCGCTTCTCACCTGAATGCCCCAACAGCTCTCTCTTA | |
| | | AACCTTCACCTACACGCCCTGCAGCCAGAAGACTCAGCCCTGTAT | |
| | | CTCTGCGCCAGCAGCCAAGGGGGGGGGGGGTACAATGAGCAGTT | |
| | | CTTCGGGCCAGGGACACGGCTCACCGTGCTAG | |
| | Variable-b | DTEVTQTPKHLVMGMTNKKSLKCEQHMGHRAMYWYKQKAKKPP | 957 |
| | | ELMFVYSYEKLSINESVPSRFSPECPNSSLLNLHLHALQPEDSALYLC | |
| | | ASSQGAGGYNEQFFGPGTRLTVL | |
| | CDR1-b | MGHRA | 639 |
| | CDR2-b | YSYEKL | 640 |
| | CDR3-b | CASSQGAGGYNEQFF | 958 |
| | FWR1-b | DTEVTQTPKHLVMGMTNKKSLKCEQH | 642 |
| | FWR2-b | MYWYKQKAKKPPELMFV | 643 |
| | FWR3-b | SINESVPSRFSPECPNSSLLNLHLHALQPEDSALYL | 644 |
| | FWR4-b | GPGTRLTVL | 9 |
| TCRA- | a-Variable | GGAGAGAATGTGGAGCAGCATCCTTCAACCCTGAGTGTCCAGGA | 959 |
| 95 | nucleotide | GGGAGACAGCGCTGTTATCAAGTGTACTTATTCAGACAGTGCCTC | |
| | | AAACTACTTCCCTTGGTATAAGCAAGAACTTGGAAAAAGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCAAGTATAGCCAAGCTCATCTTTGGGACTGGGACCAGAT | |
| | | TACAAGTCTTTCCAA | |
| | Variable-a | GENVEQHPSTLSVQEGDSAVIKCTYSDSASNYFPWYKQELGKRPQLI | 960 |
| | | IDIRSNVGEKKDQRIA VTLNKTAKHFSLHITETQPEDSAVYFCAASIA | |
| | | KLIFGTGTRLQVFP | |
| | CDR1-a | DSASNY | 207 |
| | CDR2-a | IRSNVGE | 208 |
| | CDR3-a | CAASIAKLIF | 961 |
| | FWR1-a | GENVEQHPSTLSVQEGDSAVIKCTYS | 210 |
| | FWR2-a | FPWYKQELGKRPQLIID | 211 |
| | FWR3-a | KKDQRIAVTLNKTAKHFSLHITETQPEDSAVYF | 212 |
| | FWR4-a | GTGTRLQVFP | 36 |
| TCRB- | b-Variable | GATTCTGGAGTCACACAAACCCCAAAGCACCTGATCACAGCAAC | 962 |
| 96 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCACTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCTTATCCGGGACAGGGCGAGAGACCCAGTACT | |
| | | TCGGGCCAGGCACGCGGCTCCTGGTGCTCG | |
| | Variable-b | DSGVTQTPKHLITATGQRVTLRCSPRSGDLSVYWYQQSLDQGLQFLI | 963 |
| | | HYYNGEERAKGNILERFSAQQFPDLHSELNLSSLELGDSALYFCASSL | |
| | | SGTGRETQYFGPGTRLLVL | |
| | CDR1-b | SGDLS | 383 |
| | CDR2-b | YYNGEE | 384 |
| | CDR3-b | CASSLSGTGRETQYF | 964 |
| | FWR1-b | DSGVTQTPKHLITATGQRVTLRCSPR | 386 |
| | FWR2-b | VYWYQQSLDQGLQFLIH | 424 |
| | FWR3-b | RAKGNILERFSAQQFPDLHSELNLSSLELGDSALYF | 388 |
| | FWR4-b | GPGTRLLVL | 67 |
| TCRA- | a-Variable | GAAGACCAGGTGACGCAGAGTCCCGAGGCCCTGAGACTCCAGGA | 965 |
| 96 | nucleotide | GGGAGAGAGTAGCAGTCTCAACTGCAGTTACACAGTCAGCGGTT | |
| | | TAAGAGGGCTGTTCTGGTATAGGCAAGATCCTGGGAAAGGCCCT | |
| | | GAATTCCTCTTCACCCTGTATTCAGCTGGGGAAGAAAAGGAGAA | |
| | | AGAAAGGCTAAAAGCCACATTAACAAAGAAGGAAAGCTTTCTGC | |
| | | ACATCACAGCCCCTAAACCTGAAGACTCAGCCACTTATCTCTGTG | |
| | | CTGTGCCGATAGATAGCAACTATCAGTTAATCTGGGGCGCTGGGA | |
| | | CCAAGCTAATTATAAAGCCAG | |
| | Variable-a | EDQVTQSPEALRLQEGESSSLNCSYTVSGLRGLFWYRQDPGKGPEFL | 966 |
| | | FTLYSAGEEKEKERLKATLTKKESFLHITAPKPEDSATYLCAVPIDSN | |
| | | YQLIWGAGTKLIIKP | |
| | CDR1-a | VSGLRG | 415 |
| | CDR2-a | LYSAGEE | 416 |
| | CDR3-a | CAVPIDSNYQLIW | 967 |
| | FWR1-a | EDQVTQSPEALRLQEGESSSLNCSYT | 418 |
| | FWR2-a | LFWYRQDPGKGPEFLFT | 419 |
| | FWR3-a | KEKERLKATLTKKESFLHITAPKPEDSATYL | 420 |
| | FWR4-a | GAGTKLIIKP | 356 |
| TCRB- | b-Variable | GAAGCTGGAGTTACTCAGTTCCCCAGCCACAGCGTAATAGAGAA | 968 |
| 97 | nucleotide | GGGCCAGACTGTGACTCTGAGATGTGACCCAATTTCTGGACATGA | |
| | | TAATCTTTATTGGTATCGACGTGTTATGGGAAAAGAAATAAAATT | |
| | | TCTGTTACATTTTGTGAAAGAGTCTAAACAGGATGAGTCCGGTAT | |
| | | GCCCAACAATCGATTCTTAGCTGAAAGGACTGGAGGGACGTATTC | |
| | | TACTCTGAAGGTGCAGCCTGCAGAACTGGAGGATTCTGGAGTTTA | |
| | | TTTCTGTGCCAGCAGCCCGTACTTTGGGTCCTACAATGAGCAGTT | |
| | | CTTCGGGCCAGGGACACGGCTCACCGTGCTAG | |
| | Variable-b | EAGVTQFPSHSVIEKGQTVTLRCDPISGHDNLYWYRRVMGKEIKFLL | 969 |
| | | HFVKESKQDESGMPNNRFLAERTGGTYSTLKVQPAELEDSGVYFCA | |
| | | SSPYFGSYNEQFFGPGTRLTVL | |
| | CDR1-b | SGHDN | 57 |
| | CDR2-b | FVKESK | 58 |
| | CDR3-b | CASSPYFGSYNEQFF | 970 |
| | FWR1-b | EAGVTQFPSHSVIEKGQTVTLRCDPI | 60 |
| | FWR2-b | LYWYRRVMGKEIKFLLH | 61 |
| | FWR3-b | QDESGMPNNRFLAERTGGTYSTLKVQPAELEDSGVYF | 62 |
| | FWR4-b | GPGTRLTVL | 9 |
| TCRA- | a-Variable | GAGCTGAAAGTGGAACAAAACCCTCTGTTCCTGAGCATGCAGGA | 971 |
| 97 | nucleotide | GGGAAAAAACTATACCATCTACTGCAATTATTCAACCACTTCAGA | |
| | | CAGACTGTATTGGTACAGGCAGGATCCTGGGAAAAGTCTGGAAT | |
| | | CTCTGTTTGTGTTGCTATCAAATGGAGCAGTGAAGCAGGAGGGAC | |
| | | GATTAATGGCCTCACTTGATACCAAAGCCCGTCTCAGCACCCTCC | |
| | | ACATCACAGCTGCCGTGCATGACCTCTCTGCCACCTACTTCTGTG | |
| | | CCGTGGACATGCGGCAGGGAGCCCAGAAGCTGGTATTTGGCCAA | |
| | | GGAACCAGGCTGACTATCAACCCAA | |
| | Variable-a | ELKVEQNPLFLSMQEGKNYTIYCNYSTTSDRLYWYRQDPGKSLESLF | 972 |
| | | VLLSNGAVKQEGRLMASLDTKARLSTLHITAAVHDLSATYFCAVDM | |
| | | RQGAQKLVFGQGTRLTINP | |
| | CDR1-a | TTSDR | 973 |
| | CDR2-a | LLSNGAV | 974 |
| | CDR3-a | CAVDMRQGAQKLVF | 975 |
| | FWR1-a | ELKVEQNPLFLSMQEGKNYTIYCNYS | 976 |
| | FWR2-a | LYWYRQDPGKSLESLFV | 977 |
| | FWR3-a | KQEGRLMASLDTKARLSTLHITAAVHDLSATYF | 978 |
| | FWR4-a | GQGTRLTINP | 867 |
| TCRB- | b-Variable | AAGGCTGGAGTCACTCAAACTCCAAGATATCTGATCAAAACGAG | 979 |
| 98 | nucleotide | AGGACAGCAAGTGACACTGAGCTGCTCCCCTATCTCTGGGCATAG | |
| | | GAGTGTATCCTGGTACCAACAGACCCCAGGACAGGGCCTTCAGTT | |
| | | CCTCTTTGAATACTTCAGTGAGACACAGAGAAACAAAGGAAACT | |
| | | TCCCTGGTCGATTCTCAGGGCGCCAGTTCTCTAACTCTCGCTCTGA | |
| | | GATGAATGTGAGCACCTTGGAGCTGGGGGACTCGGCCCTTTATCT | |
| | | TTGCGCCAGCAGCTTGGGGTCCTACGAGCAGTACTTCGGGCCGGG | |
| | | CACCAGGCTCACGGTCACAG | |
| | Variable-b | KAGVTQTPRYLIKTRGQQVTLSCSPISGHRSVSWYQQTPGQGLQFLF | 980 |
| | | EYFSETQRNKGNFPGRFSGRQFSNSRSEMNVSTLELGDSALYLCASS | |
| | | LGSYEQYFGPGTRLTVT | |
| | CDR1-b | SGHRS | 514 |
| | CDR2-b | YFSETQ | 515 |
| | CDR3-b | CASSLGSYEQYF | 981 |
| | FWR1-b | KAGVTQTPRYLIKTRGQQVTLSCSPI | 517 |
| | FWR2-b | VSWYQQTPGQGLQFLFE | 518 |
| | FWR3-b | RNKGNFPGRFSGRQFSNSRSEMNVSTLELGDSALYL | 519 |
| | FWR4-b | GPGTRLTVT | 85 |
| TCRA- | a-Variable | AAACAGGAGGTGACGCAGATTCCTGCAGCTCTGAGTGTCCCAGA | 982 |
| 98 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCCCCTCTCTATGGTCAGAATTTTGTCTTTGGTCCCGGAACCAGA | |
| | | TTGTCCGTGCTGCCCT | |
| | Variable-a | KQEVTQIPAALSVPEGENLVLNCSFTDSAIYNLQWFRQDPGKGLTSL | 983 |
| | | LLIQSSQREQTSGRLNASLDKSSGRSTLYIAASQPGDSATYLCAPLYG | |
| | | QNFVFGPGTRLSVLP | |
| | CDR1-a | DSAIYN | 118 |
| | CDR2-a | IQSSQRE | 119 |
| | CDR3-a | CAPLYGQNFVF | 984 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GPGTRLSVLP | 272 |
| TCRB- | b-Variable | AAGGCTGGAGTCACTCAAACTCCAAGATATCTGATCAAAACGAG | 985 |
| 99 | nucleotide | AGGACAGCAAGTGACACTGAGCTGCTCCCCTATCTCTGGGCATAG | |
| | | GAGTGTATCCTGGTACCAACAGACCCCAGGACAGGGCCTTCAGTT | |
| | | CCTCTTTGAATACTTCAGTGAGACACAGAGAAACAAAGGAAACT | |
| | | TCCCTGGTCGATTCTCAGGGCGCCAGTTCTCTAACTCTCGCTCTGA | |
| | | GATGAATGTGAGCACCTTGGAGCTGGGGGACTCGGCCCTTTATCT | |
| | | TTGCGCCAGCAGCTTGGGCAGTGGGATAGGTGAGCAGTACTTCG | |
| | | GGCCGGGCACCAGGCTCACGGTCACAG | |
| | Variable-b | KAGVTQTPRYLIKTRGQQVTLSCSPISGHRSVSWYQQTPGQGLQFLF | 986 |
| | | EYFSETQRNKGNFPGRFSGRQFSNSRSEMNVSTLELGDSALYLCASS | |
| | | LGSGIGEQYFGPGTRLTVT | |
| | CDR1-b | SGHRS | 514 |
| | CDR2-b | YFSETQ | 515 |
| | CDR3-b | CASSLGSGIGEQYF | 987 |
| | FWR1-b | KAGVTQTPRYLIKTRGQQVTLSCSPI | 517 |
| | FWR2-b | VSWYQQTPGQGLQFLFE | 518 |
| | FWR3-b | RNKGNFPGRFSGRQFSNSRSEMNVSTLELGDSALYL | 519 |
| | FWR4-b | GPGTRLTVT | 85 |
| TCRA- | a-Variable | GCCCAGAGAGTGACTCAGCCCGAGAAGCTCCTCTCTGTCTTTAAA | 988 |
| 99 | nucleotide | GGGGCCCCAGTGGAGCTGAAGTGCAACTATTCCTATTCTGGGAGT | |
| | | CCTGAACTCTTCTGGTATGTCCAGTACTCCAGACAACGCCTCCAG | |
| | | TTACTCTTGAGACACATCTCTAGAGAGAGCATCAAAGGCTTCACT | |
| | | GCTGACCTTAACAAAGGCGAGACATCTTTCCACCTGAAGAAACC | |
| | | ATTTGCTCAAGAGGAAGACTCAGCCATGTATTACTGTGCTCTAAT | |
| | | TATGTACACCGGTAACCAGTTCTATTTTGGGACAGGGACAAGTTT | |
| | | GACGGTCATTCCAA | |
| | Variable-a | AQRVTQPEKLLSVFKGAPVELKCNYSYSGSPELFWYVQYSRQRLQL | 989 |
| | | LLRHISRESIKGFTADLNKGETSFHLKKPFAQEEDSAMYYCALIMYT | |
| | | GNQFYFGTGTSLTVIP | |
| | CDR1-a | YSGSPE | 70 |
| | CDR2-a | HISR | 71 |
| | CDR3-a | CALIMYTGNQFYF | 990 |
| | FWR1-a | AQRVTQPEKLLSVFKGAPVELKCNYS | 73 |
| | FWR2-a | LFWYVQYSRQRLQLLLR | 74 |
| | FWR3-a | ESIKGFTADLNKGETSFHLKKPFAQEEDSAMYY | 75 |
| | FWR4-a | GTGTSLTVIP | 94 |
| TCRB- | b-Variable | GATTCTGGAGTCACACAAACCCCAAAGCACCTGATCACAGCAAC | 991 |
| 100 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCACTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCGGGGGACAAACCGTCTATGGCTACACCTTCGG | |
| | | TTCGGGGACCAGGTTAACCGTTGTAG | |
| | Variable-b | DSGVTQTPKHLITATGQRVTLRCSPRSGDLSVYWYQQSLDQGLQFLI | 992 |
| | | HYYNGEERAKGNILERFSAQQFPDLHSELNLSSLELGDSALYFCASS | |
| | | GGQTVYGYTFGSGTRLTVV | |
| | CDR1-b | SGDLS | 383 |
| | CDR2-b | YYNGEE | 384 |
| | CDR3-b | CASSGGQTVYGYTF | 993 |
| | FWR1-b | DSGVTQTPKHLITATGQRVTLRCSPR | 386 |
| | FWR2-b | VYWYQQSLDQGLQFLIH | 424 |
| | FWR3-b | RAKGNILERFSAQQFPDLHSELNLSSLELGDSALYF | 388 |
| | FWR4-b | GSGTRLTVV | 103 |
| TCRA- | a-Variable | GGAATACAAGTGGAGCAGAGTCCTCCAGACCTGATTCTCCAGGA | 994 |
| 100 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTGTGG | |
| | | CCGGATAGCAGCTATAAATTGATCTTCGGGAGTGGGACCAGACT | |
| | | GCTGGTCAGGCCTG | |
| | Variable-a | GIQVEQSPPDLILQEGANSTLRCNFSDSVNNLQWFHQNPWGQLINLF | 995 |
| | | YIPSGTKQNGRLSATTVATERYSLLYISSSQTTDSGVYFCAVWPDSSY | |
| | | KLIFGSGTRLL VRP | |
| | CDR1-a | DSVNN | 506 |
| | CDR2-a | IPSGT | 507 |
| | CDR3-a | CAVWPDSSYKLIF | 996 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GSGTRLLVRP | 539 |
| TCRB- | b-Variable | GAAGCCCAAGTGACCCAGAACCCAAGATACCTCATCACAGTGAC | 997 |
| 101 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTTTATCCCCTGGACAGGGCCCTATGGGCGACG | |
| | | AGCAGTACTTCGGGCCGGGCACCAGGCTCACGGTCACAG | |
| | Variable-b | EAQVTQNPRYLITVTGKKLTVTCSQNMNHEYMSWYRQDPGLGLRQ | 998 |
| | | IYYSMNVEVTDKGDVPEGYKVSRKEKRNFPLILESPSPNQTSLYFCA | |
| | | SSLSPGQGPMGDEQYFGPGTRLTVT | |
| | CDR1-b | MNHEY | 252 |
| | CDR2-b | SMNVEV | 366 |
| | CDR3-b | CASSLSPGQGPMGDEQYF | 999 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GPGTRLTVT | 85 |
| TCRA- | a-Variable | AAACAGGAGGTGACGCAGATTCCTGCAGCTCTGAGTGTCCCAGA | 1000 |
| 101 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGGTTTTACCTCAGGAACCTACAAATACATCTTTGGAACAGG | |
| | | CACCAGGCTGAAGGTTTTAGCAA | |
| | Variable-a | KQEVTQIPAALSVPEGENLVLNCSFTDSAIYNLQWFRQDPGKGLTSL | 1001 |
| | | LLIQSSQREQTSGRLNASLDKSSGRSTLYIAASQPGDSATYLCAGFTS | |
| | | GTYKYIFGTGTRLKVLA | |
| | CDR1-a | DSAIYN | 118 |
| | CDR2-a | IQSSQRE | 119 |
| | CDR3-a | CAGFTSGTYKYIF | 1002 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GTGTRLKVLA | 587 |
| TCRB- | b-Variable | GGTGCTGGAGTCTCCCAGTCTCCCAGGTACAAAGTCACAAAGAG | 1003 |
| 102 | nucleotide | GGGACAGGATGTAACTCTCAGGTGTGATCCAATTTCGAGTCATGC | |
| | | AACCCTTTATTGGTATCAACAGGCCCTGGGGCAGGGCCCAGAGTT | |
| | | TCTGACTTACTTCAATTATGAAGCTCAACCAGACAAATCAGGGCT | |
| | | GCCCAGTGATCGGTTCTCTGCAGAGAGGCCTGAGGGATCCATCTC | |
| | | CACTCTGACGATTCAGCGCACAGAGCAGCGGGACTCAGCCATGT | |
| | | ATCGCTGTGCTAGCAGCCCTTGGACTAGCGGGAGTATTGAGCAGT | |
| | | TCTTCGGGCCAGGGACACGGCTCACCGTGCTAG | |
| | Variable-b | GAGVSQSPRYKVTKRGQDVTLRCDPISSHATLYWYQQALGQGPEFL | 1004 |
| | | TYFNYEAQPDKSGLPSDRFSAERPEGSISTLTIQRTEQRDSAMYRCAS | |
| | | SPWTSGSIEQFFGPGTRLTVL | |
| | CDR1-b | SSHAT | 1005 |
| | CDR2-b | FNYEAQ | 276 |
| | CDR3-b | CASSPWTSGSIEQFF | 1006 |
| | FWR1-b | GAGVSQSPRYKVTKRGQDVTLRCDPI | 1007 |
| | FWR2-b | LYWYQQALGQGPEFLTY | 1008 |
| | FWR3-b | PDKSGLPSDRFSAERPEGSISTLTIQRTEQRDSAMYR | 1009 |
| | FWR4-b | GPGTRLTVL | 9 |
| TCRA- | a-Variable | GGAATACAAGTGGAGCAGAGTCCTCCAGACCTGATTCTCCAGGA | 1010 |
| 102 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTTGAC | |
| | | TCAGGAACCTACAAATACATCTTTGGAACAGGCACCAGGCTGAA | |
| | | GGTTTTAGCAA | |
| | Variable-a | GIQVEQSPPDLILQEGANSTLRCNFSDSVNNLQWFHQNPWGQLINLF | 1011 |
| | | YIPSGTKQNGRLSATTVATERYSLLYISSSQTTDSGVYFCAVDSGTYK | |
| | | YIFGTGTRLKVLA | |
| | CDR1-a | DSVNN | 506 |
| | CDR2-a | IPSGT | 507 |
| | CDR3-a | CAVDSGTYKYIF | 1012 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GTGTRLKVLA | 587 |
| TCRB- | b-Variable | AGTGCTGTCATCTCTCAAAAGCCAAGCAGGGATATCTGTCAACGT | 1013 |
| 103 | nucleotide | GGAACCTCCCTGACGATCCAGTGTCAAGTCGATAGCCAAGTCACC | |
| | | ATGATGTTCTGGTACCGTCAGCAACCTGGACAGAGCCTGACACTG | |
| | | ATCGCAACTGCAAATCAGGGCTCTGAGGCCACATATGAGAGTGG | |
| | | ATTTGTCATTGACAAGTTTCCCATCAGCCGCCCAAACCTAACATT | |
| | | CTCAACTCTGACTGTGAGCAACATGAGCCCTGAAGACAGCAGCA | |
| | | TATATCTCTGCAGCGTTGTGGGTGATCACGAGCAGTACTTCGGGC | |
| | | CGGGCACCAGGCTCACGGTCACAG | |
| | Variable-b | SAVISQKPSRDICQRGTSLTIQCQVDSQVTMMFWYRQQPGQSLTLIA | 1014 |
| | | TANQGSEATYESGFVIDKFPISRPNLTFSTLTVSNMSPEDSSIYLCSVV | |
| | | GDHEQYFGPGTRLTVT | |
| | CDR1-b | SQVTM | 97 |
| | CDR2-b | ANQGSEA | 98 |
| | CDR3-b | CSVVGDHEQYF | 1015 |
| | FWR1-b | SAVISQKPSRDICQRGTSLTIQCQVD | 100 |
| | FWR2-b | MFWYRQQPGQSLTLIAT | 101 |
| | FWR3-b | TYESGFVIDKFPISRPNLTFSTLTVSNMSPEDSSIYL | 102 |
| | FWR4-b | GPGTRLTVT | 85 |
| TCRA- | a-Variable | CAGAAGGAGGTGGAGCAGAATTCTGGACCCCTCAGTGTTCCAGA | 1016 |
| 103 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTTCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTCCCCGGGGGGAAAAACAATGACATGCGCTTTGGAGCAGGG | |
| | | ACCAGACTGACAGTAAAACCAA | |
| | Variable-a | QKEVEQNSGPLSVPEGAIASLNCTYSDRGSQSFFWYRQYSGKSPELI | 1017 |
| | | MFIYSNGDKEDGRFTAQLNKASQYVSLLIRDSQPSDSATYLCAVPGG | |
| | | KNNDMRFGAGTRLTVKP | |
| | CDR1-a | DRGSQS | 12 |
| | CDR2-a | IYSNGD | 13 |
| | CDR3-a | CAVPGGKNNDMRF | 1018 |
| | FWR1-a | QKEVEQNSGPLSVPEGAIASLNCTYS | 15 |
| | FWR2-a | FFWYRQYSGKSPELIMF | 16 |
| | FWR3-a | KEDGRFTAQLNKASQYVSLLIRDSQPSDSATYL | 17 |
| | FWR4-a | GAGTRLTVKP | 685 |
| TCRB- | b-Variable | GATGTGAAAGTAACCCAGAGCTCGAGATATCTAGTCAAAAGGAC | 679 |
| 104 | nucleotide | GGGAGAGAAAGTTTTTCTGGAATGTGTCCAGGATATGGACCATG | |
| | | AAAATATGTTCTGGTATCGACAAGACCCAGGTCTGGGGCTACGGC | |
| | | TGATCTATTTCTCATATGATGTTAAAATGAAAGAAAAAGGAGATA | |
| | | TTCCTGAGGGGTACAGTGTCTCTAGAGAGAAGAAGGAGCGCTTCT | |
| | | CCCTGATTCTGGAGTCCGCCAGCACCAACCAGACATCTATGTACC | |
| | | TCTGTGCCAGCAGTTGGACAGAAGAGACCTACGAGCAGTACTTC | |
| | | GGGCCGGGCACCAGGCTCACGGTCACAG | |
| | Variable-b | DVKVTQSSRYLVKRTGEKVFLECVQDMDHENMFWYRQDPGLGLRL | 680 |
| | | IYFSYDVKMKEKGDIPEGYSVSREKKERFSLILESASTNQTSMYLCAS | |
| | | SWTEETYEQYFGPGTRLTVT | |
| | CDR1-b | MDHEN | 199 |
| | CDR2-b | SYDVKM | 200 |
| | CDR3-b | CASSWTEETYEQYF | 681 |
| | FWR1-b | DVKVTQSSRYLVKRTGEKVFLECVQD | 202 |
| | FWR2-b | MFWYRQDPGLGLRLIYF | 203 |
| | FWR3-b | KEKGDIPEGYSVSREKKERFSLILESASTNQTSMYL | 204 |
| | FWR4-b | GPGTRLTVT | 85 |
| TCRA- | a-Variable | GCTCAGTCAGTGGCTCAGCCGGAAGATCAGGTCAACGTTGCTGA | 682 |
| 104 | nucleotide | AGGGAATCCTCTGACTGTGAAATGCACCTATTCAGTCTCTGGAAA | |
| | | CCCTTATCTTTTTTGGTATGTTCAATACCCCAACCGAGGCCTCCAG | |
| | | TTCCTTCTGAAATACATCACAGGGGATAACCTGGTTAAAGGCAGC | |
| | | TATGGCTTTGAAGCTGAATTTAACAAGAGCCAAACCTCCTTCCAC | |
| | | CTGAAGAAACCATCTGCCCTTGTGAGCGACTCCGCTTTGTACTTC | |
| | | TGTGCTGTGAGAGCGTACAATAACAATGACATGCGCTTTGGAGCA | |
| | | GGGACCAGACTGACAGTAAAACCAA | |
| | Variable-a | AQSVAQPEDQVNVAEGNPLTVKCTYSVSGNPYLFWYVQYPNRGLQ | 683 |
| | | FLLKYITGDNLVKGSYGFEAEFNKSQTSFHLKKPSALVSDSALYFCA | |
| | | VRAYNNNDMRFGAGTRLTVKP | |
| | CDR1-a | VSGNPY | 427 |
| | CDR2-a | YITGDNLV | 428 |
| | CDR3-a | CAVRAYNNNDMRF | 684 |
| | FWR1-a | AQSVAQPEDQVNVAEGNPLTVKCTYS | 430 |
| | FWR2-a | LFWYVQYPNRGLQFLLK | 431 |
| | FWR3-a | KGSYGFEAEFNKSQTSFHLKKPSALVSDSALYF | 432 |
| | FWR4-a | GAGTRLTVKP | 685 |
| TCRB- | b-Variable | GATTCTGGAGTCACACAAACCCCAAAGCACCTGATCACAGCAAC | 1019 |
| 105 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCCCGAAGGACGTCAGTACTGGGGCCAACGTCC | |
| | | TGACTTTCGGGGCCGGCAGCAGGCTGACCGTGCTGG | |
| | Variable-b | DSGVTQTPKHLITATGQRVTLRCSPRSGDLSVYWYQQSLDQGLQFLI | 1020 |
| | | QYYNGEERAKGNILERFSAQQFPDLHSELNLSSLELGDSALYFCASSP | |
| | | KDVSTGANVLTFGAGSRLTVL | |
| | CDR1-b | SGDLS | 383 |
| | CDR2-b | YYNGEE | 384 |
| | CDR3-b | CASSPKDVSTGANVLTF | 1021 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GAGSRLTVL | 816 |
| TCRA- | a-Variable | GATGCTAAGACCACACAGCCAAATTCAATGGAGAGTAACGAAGA | 1022 |
| 105 | nucleotide | AGAGCCTGTTCACTTGCCTTGTAACCACTCCACAATCAGTGGAAC | |
| | | TGATTACATACATTGGTATCGACAGCTTCCCTCCCAGGGTCCAGA | |
| | | GTACGTGATTCATGGTCTTACAAGCAATGTGAACAACAGAATGGC | |
| | | CTCTCTGGCAATCGCTGAAGACAGAAAGTCCAGTACCTTGATCCT | |
| | | GCACCGTGCTACCTTGAGAGATGCTGCTGTGTACTACTGCATCCT | |
| | | GAAAACCTCCTACGACAAGGTGATATTTGGGCCAGGGACAAGCT | |
| | | TATCAGTCATTCCAA | |
| | Variable-a | DAKTTQPNSMESNEEEPVHLPCNHSTISGTDYIHWYRQLPSQGPEYV | 1023 |
| | | IHGLTSNVNNRMASLAIAEDRKSSTLILHRATLRDAAVYYCILKTSY | |
| | | DKVIFGPGTSLSVIP | |
| | CDR1-a | TISGTDY | 153 |
| | CDR2-a | GLTSN | 154 |
| | CDR3-a | CILKTSYDKVIF | 1024 |
| | FWR1-a | DAKTTQPNSMESNEEEPVHLPCNHS | 156 |
| | FWR2-a | IHWYRQLPSQGPEYVIH | 157 |
| | FWR3-a | VNNRMASLAIAEDRKSSTLILHRATLRDAAVYY | 158 |
| | FWR4-a | GPGTSLSVIP | 1025 |
| TCRB- | b-Variable | GAAGCCCAAGTGACCCAGAACCCAAGATACCTCATCACAGTGAC | 1026 |
| 106 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTTTAGAGGGTAGCGGAAGTGCCGGGGAGCTG | |
| | | TTTTTTGGAGAAGGCTCTAGGCTGACCGTACTGG | |
| | Variable-b | EAQVTQNPRYLITVTGKKLTVTCSQNMNHEYMSWYRQDPGLGLRQ | 1027 |
| | | IYYSMNVEVTDKGDVPEGYKVSRKEKRNFPLILESPSPNQTSLYFCA | |
| | | SSLEGSGSAGELFFGEGSRLTVL | |
| | CDR1-b | MNHEY | 252 |
| | CDR2-b | SMNVEV | 366 |
| | CDR3-b | CASSLEGSGSAGELFF | 1028 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GEGSRLTVL | 63 |
| TCRA- | a-Variable | AGCCAAAAGATAGAACAGAATTCCGAGGCCCTGAACATTCAGGA | 1029 |
| 106 | nucleotide | GGGTAAAACGGCCACCCTGACCTGCAACTATACAAACTATTCTCC | |
| | | AGCATACTTACAGTGGTACCGACAAGATCCAGGAAGAGGCCCTG | |
| | | TTTTCTTGCTACTCATACGTGAAAATGAGAAAGAAAAAAGGAAA | |
| | | GAAAGACTGAAGGTCACCTTTGATACCACCCTTAAACAGAGTTTG | |
| | | TTTCATATCACAGCCTCCCAGCCTGCAGACTCAGCTACCTACCTCT | |
| | | GTGCTCTAGAAGAAACTGACAGCTGGGGGAAATTGCAGTTTGGA | |
| | | GCAGGGACCCAGGTTGTGGTCACCCCAG | |
| | Variable-a | SQKIEQNSEALNIQEGKTATLTCNYTNYSPAYLQWYRQDPGRGPVFL | 1030 |
| | | LLIRENEKEKRKERLKVTFDTTLKQSLFHITASQPADSATYLCALEET | |
| | | DSWGKLQFGAGTQVVVTP | |
| | CDR1-a | NYSPAY | 831 |
| | CDR2-a | IRENEKE | 832 |
| | CDR3-a | CALEETDSWGKLQF | 1031 |
| | FWR1-a | SQKIEQNSEALNIQEGKTATLTCNYT | 834 |
| | FWR2-a | LQWYRQDPGRGPVFLLL | 835 |
| | FWR3-a | KRKERLKVTFDTTLKQSLFHITASQPADSATYL | 836 |
| | FWR4-a | GAGTQVVVTP | 54 |
| TCRB- | b-Variable | GATTCTGGAGTCACACAAACCCCAAAGCACCTGATCACAGCAAC | 1032 |
| 107 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCCGGCGAAGCTTGAGCTCCTACAATGAGCAGTTCTT | |
| | | CGGGCCAGGGACACGGCTCACCGTGCTAG | |
| | Variable-b | DSGVTQTPKHLITATGQRVTLRCSPRSGDLSVYWYQQSLDQGLQFLI | 1033 |
| | | QYYNGEERAKGNILERFSAQQFPDLHSELNLSSLELGDSALYFCASR | |
| | | RSLSSYNEQFFGPGTRLTVL | |
| | CDR1-b | SGDLS | 383 |
| | CDR2-b | YYNGEE | 384 |
| | CDR3-b | CASRRSLSSYNEQFF | 1034 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GPGTRLTVL | 9 |
| TCRA- | a-Variable | GACCAGCAAGTTAAGCAAAATTCACCATCCCTGAGCGTCCAGGA | 1035 |
| 107 | nucleotide | AGGAAGAATTTCTATTCTGAACTGTGACTATACTAACAGCATGTT | |
| | | TGATTATTTCCTATGGTACAAAAAATACCCTGCTGAAGGTCCTAC | |
| | | ATTCCTGATATCTATAAGTTCCATTAAGGATAAAAATGAAGATGG | |
| | | AAGATTCACTGTTTTCTTAAACAAAAGTGCCAAGCACCTCTCTCT | |
| | | GCACATTGTGCCCTCCCAGCCTGGAGACTCTGCAGTGTACTTCTG | |
| | | TGCAGCAACCTATAATAATGCAGGCAACATGCTCACCTTTGGAGG | |
| | | GGGAACAAGGTTAATGGTCAAACCCC | |
| | Variable-a | DQQVKQNSPSLSVQEGRISILNCDYTNSMFDYFLWYKKYPAEGPTFL | 1036 |
| | | ISISSIKDKNEDGRFTVFLNKSAKHLSLHIVPSQPGDSA VYFCAATYN | |
| | | NAGNMLTFGGGTRLMVKP | |
| | CDR1-a | NSMFDY | 303 |
| | CDR2-a | ISSIKDK | 304 |
| | CDR3-a | CAATYNNAGNMLTF | 1037 |
| | FWR1-a | DQQVKQNSPSLSVQEGRISILNCDYT | 306 |
| | FWR2-a | FLWYKKYPAEGPTFLIS | 307 |
| | FWR3-a | NEDGRFTVFLNKSAKHLSLHIVPSQPGDSAVYF | 308 |
| | FWR4-a | GGGTRLMVKP | 141 |
| TCRB- | b-Variable | GATTCTGGAGTCACACAAACCCCAAAGCACCTGATCACAGCAAC | 1038 |
| 108 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCGTAGGGAGTGTGGGGGGACTAGCGGACTTCA | |
| | | ATGAGCAGTTCTTCGGGCCAGGGACACGGCTCACCGTGCTAG | |
| | Variable-b | DSGVTQTPKHLITATGQRVTLRCSPRSGDLSVYWYQQSLDQGLQFLI | 1039 |
| | | QYYNGEERAKGNILERFSAQQFPDLHSELNLSSLELGDSALYFCASS | |
| | | VGSVGGLADFNEQFFGPGTRLTVL | |
| | CDR1-b | SGDLS | 383 |
| | CDR2-b | YYNGEE | 384 |
| | CDR3-b | CASSVGSVGGLADFNEQFF | 1040 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GPGTRLTVL | 9 |
| TCRA- | a-Variable | GCCCAGAAGATAACTCAAACCCAACCAGGAATGTTCGTGCAGGA | 1041 |
| 108 | nucleotide | AAAGGAGGCTGTGACTCTGGACTGCACATATGACACCAGTGATC | |
| | | AAAGTTATGGTCTATTCTGGTACAAGCAGCCCAGCAGTGGGGAA | |
| | | ATGATTTTTCTTATTTATCAGGGGTCTTATGACGAGCAAAATGCA | |
| | | ACAGAAGGTCGCTACTCATTGAATTTCCAGAAGGCAAGAAAATC | |
| | | CGCCAACCTTGTCATCTCCGCTTCACAACTGGGGGACTCAGCAAT | |
| | | GTATTTCTGTGCAATGAGAGAGGGCTGGGTTAGTGACATGCGCTT | |
| | | TGGAGCAGGGACCAGACTGACAGTAAAACCAA | |
| | Variable-a | AQKITQTQPGMFVQEKEAVTLDCTYDTSDQSYGLFWYKQPSSGEMI | 1042 |
| | | FLIYQGSYDEQNATEGRYSLNFQKARKSANLVISASQLGDSAMYFC | |
| | | AMREGWVSDMRFGAGTRLTVKP | |
| | CDR1-a | TSDQSYG | 403 |
| | CDR2-a | QGSYDEQN | 404 |
| | CDR3-a | CAMREGWVSDMRF | 1043 |
| | FWR1-a | AQKITQTQPGMFVQEKEAVTLDCTYD | 406 |
| | FWR2-a | LFWYKQPSSGEMIFLIY | 407 |
| | FWR3-a | ATEGRYSLNFQKARKSANLVISASQLGDSAMYF | 408 |
| | FWR4-a | GAGTRLTVKP | 685 |
| TCRB- | b-Variable | AATGCTGGTGTCACTCAGACCCCAAAATTCCGGGTCCTGAAGACA | 1044 |
| 109 | nucleotide | GGACAGAGCATGACACTGCTGTGTGCCCAGGATATGAACCATGA | |
| | | ATACATGTACTGGTATCGACAAGACCCAGGCATGGGGCTGAGGC | |
| | | TGATTCATTACTCAGTTGGTGAGGGTACAACTGCCAAAGGAGAG | |
| | | GTCCCTGATGGCTACAATGTCTCCAGATTAAAAAAACAGAATTTC | |
| | | CTGCTGGGGTTGGAGTCGGCTGCTCCCTCCCAAACATCTGTGTAC | |
| | | TTCTGTGCCAGCAGTTCCCCTTTCTACGAGCAGTACTTCGGGCCG | |
| | | GGCACCAGGCTCACGGTCACAG | |
| | Variable-b | NAGVTQTPKFRVLKTGQSMTLLCAQDMNHEYMYWYRQDPGMGLR | 1045 |
| | | LIHYSVGEGTTAKGEVPDGYNVSRLKKQNFLLGLESAAPSQTSVYFC | |
| | | ASSSPFYEQYFGPGTRLTVT | |
| | CDR1-b | MNHEY | 252 |
| | CDR2-b | SVGEGT | 1046 |
| | CDR3-b | CASSSPFYEQYF | 1047 |
| | FWR1-b | NAGVTQTPKFRVLKTGQSMTLLCAQD | 1048 |
| | FWR2-b | MYWYRQDPGMGLRLIHY | 1049 |
| | FWR3-b | TAKGEVPDGYNVSRLKKQNFLLGLESAAPSQTSVYF | 1050 |
| | FWR4-b | GPGTRLTVT | 85 |
| TCRA- | a-Variable | GGAATACAAGTGGAGCAGAGTCCTCCAGACCTGATTCTCCAGGA | 1051 |
| 109 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTGGA | |
| | | AGAATGGGGAAGCCAAGGAAATCTCATCTTTGGAAAAGGCACTA | |
| | | AACTCTCTGTTAAACCAA | |
| | Variable-a | GIQVEQSPPDLILQEGANSTLRCNFSDSVNNLQWFHQNPWGQLINLF | 1052 |
| | | YIPSGTKQNGRLSATTVATERYSLLYISSSQTTDSGVYFCAVEEWGS | |
| | | QGNLIFGKGTKLSVKP | |
| | CDR1-a | DSVNN | 506 |
| | CDR2-a | IPSGT | 507 |
| | CDR3-a | CAVEEWGSQGNLIF | 1053 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GKGTKLSVKP | 124 |
| TCRB- | b-Variable | GAAACGGGAGTTACGCAGACACCAAGACACCTGGTCATGGGAAT | 1054 |
| 110 | nucleotide | GACAAATAAGAAGTCTTTGAAATGTGAACAACATCTGGGTCATA | |
| | | ACGCTATGTATTGGTACAAGCAAAGTGCTAAGAAGCCACTGGAG | |
| | | CTCATGTTTGTCTACAGTCTTGAAGAACGGGTTGAAAACAACAGT | |
| | | GTGCCAAGTCGCTTCTCACCTGAATGCCCCAACAGCTCTCACTTA | |
| | | TTCCTTCACCTACACACCCTGCAGCCAGAAGACTCGGCCCTGTAT | |
| | | CTCTGCGCCAGCAGCCAAGGAAGGGCGTTTCTGGGCCAGGGGTT | |
| | | GTACGAGCAGTACTTCGGGCCGGGCACCAGGCTCACGGTCACAG | |
| | Variable-b | ETGVTQTPRHLVMGMTNKKSLKCEQHLGHNAMYWYKQSAKKPLE | 1055 |
| | | LMFVYSLEERVENNSVPSRFSPECPNSSHLFLHLHTLQPEDSALYLCA | |
| | | SSQGRAFLGQGLYEQYFGPGTRLTVT | |
| | CDR1-b | LGHNA | 169 |
| | CDR2-b | YSLEER | 706 |
| | CDR3-b | CASSQGRAFLGQGLYEQYF | 1056 |
| | FWR1-b | ETGVTQTPRHLVMGMTNKKSLKCEQH | 172 |
| | FWR2-b | MYWYKQSAKKPLELMFV | 173 |
| | FWR3-b | VENNSVPSRFSPECPNSSHLFLHLHTLQPEDSALYL | 708 |
| | FWR4-b | GPGTRLTVT | 85 |
| TCRA- | a-Variable | ACCCAGCTGCTGGAGCAGAGCCCTCAGTTTCTAAGCATCCAAGAG | 1057 |
| 110 | nucleotide | GGAGAAAATCTCACTGTGTACTGCAACTCCTCAAGTGTTTTTTCC | |
| | | AGCTTACAATGGTACAGACAGGAGCCTGGGGAAGGTCCTGTCCT | |
| | | CCTGGTGACAGTAGTTACGGGTGGAGAAGTGAAGAAGCTGAAGA | |
| | | GACTAACCTTTCAGTTTGGTGATGCAAGAAAGGACAGTTCTCTCC | |
| | | ACATCACTGCAGCCCAGCCTGGTGATACAGGCCTCTACCTCTGTG | |
| | | CAGGAGTAGGAAGAGATGACAAGATCATCTTTGGAAAAGGGACA | |
| | | CGACTTCATATTCTCCCCA | |
| | Variable-a | TQLLEQSPQFLSIQEGENLTVYCNSSSVFSSLQWYRQEPGEGPVLLVT | 1058 |
| | | VVTGGEVKKLKRLTFQFGDARKDSSLHITAAQPGDTGLYLCAGVGR | |
| | | DDKIIFGKGTRLHILP | |
| | CDR1-a | SVFSS | 533 |
| | CDR2-a | VVTGGEV | 534 |
| | CDR3-a | CAGVGRDDKIIF | 1059 |
| | FWR1-a | TQLLEQSPQFLSIQEGENLTVYCNSS | 536 |
| | FWR2-a | LQWYRQEPGEGPVLLVT | 537 |
| | FWR3-a | KKLKRLTFQFGDARKDSSLHITAAQPGDTGLYL | 538 |
| | FWR4-a | GKGTRLHILP | 607 |
| TCRB- | b-Variable | GGTGCTGTCGTCTCTCAACATCCGAGCAGGGTTATCTGTAAGAGT | 1060 |
| 111 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGAGATCGGGGGGCCTACGAGCAGT | |
| | | ACTTCGGGCCGGGCACCAGGCTCACGGTCACAG | |
| | Variable-b | GAVVSQHPSRVICKSGTSVKIECRSLDFQATTMFWYRQFPKQSLML | 1061 |
| | | MATSNEGSKATYEQGVEKDKFLINHASLTLSTLTVTSAHPEDSSFYIC | |
| | | SARDRGAYEQYFGPGTRLTVT | |
| | CDR1-b | DFQATT | 79 |
| | CDR2-b | SNEGSKA | 80 |
| | CDR3-b | CSARDRGAYEQYF | 1062 |
| | FWR1-b | GAVVSQHPSRVICKSGTSVKIECRSL | 1063 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVT | 85 |
| TCRA- | a-Variable | GGAAATTCAGTGACCCAGATGGAAGGGCCAGTGACTCTCTCAGA | 1064 |
| 111 | nucleotide | AGAGGCCTTCCTGACTATAAACTGCACGTACACAGCCACAGGAT | |
| | | ACCCTTCCCTTTTCTGGTATGTCCAATATCCTGGAGAAGGTCTACA | |
| | | GCTCCTCCTGAAAGCCACGAAGGCTGATGACAAGGGAAGCAACA | |
| | | AAGGTTTTGAAGCCACATACCGTAAAGAAACCACTTCTTTCCACT | |
| | | TGGAGAAAGGCTCAGTTCAAGTGTCAGACTCAGCGGTGTACTTCT | |
| | | GTGCTCTGAGTGATCTGAATAACAATGCCAGACTCATGTTTGGAG | |
| | | ATGGAACTCAGCTGGTGGTGAAGCCCA | |
| | Variable-a | GNSVTQMEGPVTLSEEAFLTINCTYTATGYPSLFWYVQYPGEGLQLL | 1065 |
| | | LKATKADDKGSNKGFEATYRKETTSFHLEKGSVQVSDSAVYFCALS | |
| | | DLNNNARLMFGDGTQLVVKP | |
| | CDR1-a | ATGYPS | 106 |
| | CDR2-a | ATKADDK | 107 |
| | CDR3-a | CALSDLNNNARLMF | 1066 |
| | FWR1-a | GNSVTQMEGPVTLSEEAFLTINCTYT | 724 |
| | FWR2-a | LFWYVQYPGEGLQLLLK | 110 |
| | FWR3-a | GSNKGFEATYRKETTSFHLEKGSVQVSDSAVYF | 111 |
| | FWR4-a | GDGTQLVVKP | 230 |
| TCRB- | b-Variable | GAACCTGAAGTCACCCAGACTCCCAGCCATCAGGTCACACAGAT | 1067 |
| 112 | nucleotide | GGGACAGGAAGTGATCTTGCGCTGTGTCCCCATCTCTAATCACTT | |
| | | ATACTTCTATTGGTACAGACAAATCTTGGGGCAGAAAGTCGAGTT | |
| | | TCTGGTTTCCTTTTATAATAATGAAATCTCAGAGAAGTCTGAAAT | |
| | | ATTCGATGATCAATTCTCAGTTGAAAGGCCTGATGGATCAAATTT | |
| | | CACTCTGAAGATCCGGTCCACAAAGCTGGAGGACTCAGCCATGT | |
| | | ACTTCTGTGCCAGCAGTGCGAGCAGCGGGGGGTTCTACAATGAG | |
| | | CAGTTCTTCGGGCCAGGGACACGGCTCACCGTGCTAG | |
| | Variable-b | EPEVTQTPSHQVTQMGQEVILRCVPISNHLYFYWYRQILGQKVEFLV | 1068 |
| | | SFYNNEISEKSEIFDDQFSVERPDGSNFTLKIRSTKLEDSAMYFCASSA | |
| | | SSGGFYNEQFFGPGTRLTVL | |
| | CDR1-b | SNHLY | 525 |
| | CDR2-b | FYNNEI | 526 |
| | CDR3-b | CASSASSGGFYNEQFF | 1069 |
| | FWR1-b | EPEVTQTPSHQVTQMGQEVILRCVPI | 528 |
| | FWR2-b | FYWYRQILGQKVEFLVS | 529 |
| | FWR3-b | SEKSEIFDDQFSVERPDGSNFTLKIRSTKLEDSAMYF | 530 |
| | FWR4-b | GPGTRLTVL | 9 |
| TCRA- | a-Variable | GCCCAGTCTGTGAGCCAGCATAACCACCACGTAATTCTCTCTGAA | 1070 |
| 112 | nucleotide | GCAGCCTCACTGGAGTTGGGATGCAACTATTCCTATGGTGGAACT | |
| | | GTTAATCTCTTCTGGTATGTCCAGTACCCTGGTCAACACCTTCAGC | |
| | | TTCTCCTCAAGTACTTTTCAGGGGATCCACTGGTTAAAGGCATCA | |
| | | AGGGCTTTGAGGCTGAATTTATAAAGAGTAAATTCTCCTTTAATC | |
| | | TGAGGAAACCCTCTGTGCAGTGGAGTGACACAGCTGAGTACTTCT | |
| | | GTGCCGTGAATGGGGCAGGAGGAGGTGCTGACGGACTCACCTTT | |
| | | GGCAAAGGGACTCATCTAATCATCCAGCCCT | |
| | Variable-a | AQSVSQHNHHVILSEAASLELGCNYSYGGTVNLFWYVQYPGQHLQL | 1071 |
| | | LLKYFSGDPLVKGIKGFEAEFIKSKFSFNLRKPSVQWSDTAEYFCAV | |
| | | NGAGGGADGLTFGKGTHLIIQP | |
| | CDR1-a | YGGTVN | 1072 |
| | CDR2-a | YFSGDPLV | 1073 |
| | CDR3-a | CAVNGAGGGADGLTF | 1074 |
| | FWR1-a | AQSVSQHNHHVILSEAASLELGCNYS | 1075 |
| | FWR2-a | LFWYVQYPGQHLQLLLK | 1076 |
| | FWR3-a | KGIKGFEAEFIKSKFSFNLRKPSVQWSDTAEYF | 1077 |
| | FWR4-a | GKGTHLIIQP | 363 |
| TCRB- | b-Variable | GATACTGGAGTCTCCCAGGACCCCAGACACAAGATCACAAAGAG | 1078 |
| 113 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCTTAGTGTCCGCGGTAGGGAACATTCAGT | |
| | | ACTTCGGCGCCGGGACCCGGCTCTCAGTGCTGG | |
| | Variable-b | DTGVSQDPRHKITKRGQNVTFRCDPISEHNRLYWYRQTLGQGPEFLT | 1079 |
| | | YFQNEAQLEKSRLLSDRFSAERPKGSFSTLEIQRTEQGDSAMYLCASS | |
| | | LVSAVGNIQYFGAGTRLSVL | |
| | CDR1-b | SEHNR | 442 |
| | CDR2-b | FQNEAQ | 443 |
| | CDR3-b | CASSLVSAVGNIQYF | 1080 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GAGTRLSVL | 1081 |
| TCRA- | a-Variable | CAGAAGGAGGTGGAGCAGGATCCTGGACCACTCAGTGTTCCAGA | 1082 |
| 113 | nucleotide | GGGAGCCATTGTTTCTCTCAACTGCACTTACAGCAACAGTGCTTT | |
| | | TCAATACTTCATGTGGTACAGACAGTATTCCAGAAAAGGCCCTGA | |
| | | GTTGCTGATGTACACATACTCCAGTGGTAACAAAGAAGATGGAA | |
| | | GGTTTACAGCACAGGTCGATAAATCCAGCAAGTATATCTCCTTGT | |
| | | TCATCAGAGACTCACAGCCCAGTGATTCAGCCACCTACCTCTGTG | |
| | | CAATGAGCAATTATAACACCGACAAGCTCATCTTTGGGACTGGGA | |
| | | CCAGATTACAAGTCTTTCCAA | |
| | Variable-a | QKEVEQDPGPLSVPEGAIVSLNCTYSNSAFQYFMWYRQYSRKGPEL | 1083 |
| | | LMYTYSSGNKEDGRFTAQVDKSSKYISLFIRDSQPSDSATYLCAMSN | |
| | | YNTDKLIFGTGTRLQVFP | |
| | CDR1-a | NSAFQY | 673 |
| | CDR2-a | TYSSGN | 674 |
| | CDR3-a | CAMSNYNTDKLIF | 1084 |
| | FWR1-a | QKEVEQDPGPLSVPEGAIVSLNCTYS | 676 |
| | FWR2-a | FMWYRQYSRKGPELLMY | 677 |
| | FWR3-a | KEDGRFTAQVDKSSKYISLFIRDSQPSDSATYL | 678 |
| | FWR4-a | GTGTRLQVFP | 36 |
| TCRB- | b-Variable | GATGGTGGAATCACTCAGTCCCCAAAGTACCTGTTCAGAAAGGA | 1085 |
| 114 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTACCACCGGGGGGGACAGTCTTAGCAATCAGCC | |
| | | CCAGCATTTTGGTGATGGGACTCGACTCTCCATCCTAG | |
| | Variable-b | DGGITQSPKYLFRKEGQNVTLSCEQNLNHDAMYWYRQDPGQGLRLI | 1086 |
| | | YYSQIVNDFQKGDIAEGYSVSREKKESFPLTVTSAQKNPTAFYLCAS | |
| | | TTGGDSLSNQPQHFGDGTRLSIL | |
| | CDR1-b | LNHDA | 216 |
| | CDR2-b | SQIVND | 217 |
| | CDR3-b | CASTTGGDSLSNQPQHF | 1087 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GDGTRLSIL | 27 |
| TCRA- | a-Variable | CAGAAGGAGGTGGAGCAGAATTCTGGACCCCTCAGTGTTCCAGA | 1088 |
| 114 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTTCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAAACGCCCCAGGGATGCTGGTGGTACTAGCTATGGAAAG | |
| | | CTGACATTTGGACAAGGGACCATCTTGACTGTCCATCCAA | |
| | Variable-a | QKEVEQNSGPLSVPEGAIASLNCTYSDRGSQSFFWYRQYSGKSPELI | 1089 |
| | | MFIYSNGDKEDGRFTAQLNKASQYVSLLIRDSQPSDSATYLCAVKRP | |
| | | RDAGGTSYGKLTFGQGTILTVHP | |
| | CDR1-a | DRGSQS | 12 |
| | CDR2-a | IYSNGD | 13 |
| | CDR3-a | CAVKRPRDAGGTSYGKLTF | 1090 |
| | FWR1-a | QKEVEQNSGPLSVPEGAIASLNCTYS | 15 |
| | FWR2-a | FFWYRQYSGKSPELIMF | 16 |
| | FWR3-a | KEDGRFTAQLNKASQYVSLLIRDSQPSDSATYL | 17 |
| | FWR4-a | GQGTILTVHP | 76 |
| TCRB- | b-Variable | AATGCTGGTGTCACTCAGACCCCAAAATTCCAGGTCCTGAAGACA | 1091 |
| 115 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTTACTGGACAGGGACCTACGAGCAGTACTTCGG | |
| | | GCCGGGCACCAGGCTCACGGTCACAG | |
| | Variable-b | NAGVTQTPKFQVLKTGQSMTLQCAQDMNHEYMSWYRQDPGMGLR | 1092 |
| | | LIHYSVGAGITDQGEVPNGYNVSRSTTEDFPLRLLSAAPSQTSVYFCA | |
| | | SSYWTGTYEQYFGPGTRLTVT | |
| | CDR1-b | MNHEY | 252 |
| | CDR2-b | SVGAGI | 253 |
| | CDR3-b | CASSYWTGTYEQYF | 1093 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GPGTRLTVT | 85 |
| TCRA- | a-Variable | AGTCAACAGGGAGAAGAGGATCCTCAGGCCTTGAGCATCCAGGA | 1094 |
| 115 | nucleotide | GGGTGAAAATGCCACCATGAACTGCAGTTACAAAACTAGTATAA | |
| | | ACAATTTACAGTGGTATAGACAAAATTCAGGTAGAGGCCTTGTCC | |
| | | ACCTAATTTTAATACGTTCAAATGAAAGAGAGAAACACAGTGGA | |
| | | AGATTAAGAGTCACGCTTGACACTTCCAAGAAAAGCAGTTCCTTG | |
| | | TTGATCACGGCTTCCCGGGCAGCAGACACTGCTTCTTACTTCTGT | |
| | | GCTACGGTCTATTCAGGAGGAGGTGCTGACGGACTCACCTTTGGC | |
| | | AAAGGGACTCATCTAATCATCCAGCCCT | |
| | Variable-a | SQQGEEDPQALSIQEGENATMNCSYKTSINNLQWYRQNSGRGL VHL | 1095 |
| | | ILIRSNEREKHSGRLRVTLDTSKKSSSLLITASRAADTASYFCATVYS | |
| | | GGGADGLTFGKGTHLIIQP | |
| | CDR1-a | TSINN | 552 |
| | CDR2-a | IRSNERE | 553 |
| | CDR3-a | CATVYSGGGADGLTF | 1096 |
| | FWR1-a | SQQGEEDPQALSIQEGENATMNCSYK | 555 |
| | FWR2-a | LQWYRQNSGRGLVHLIL | 556 |
| | FWR3-a | KHSGRLRVTLDTSKKSSSLLITASRAADTASYF | 557 |
| | FWR4-a | GKGTHLIIQP | 363 |
| TCRB- | b-Variable | GATGGTGGAATCACTCAGTCCCCAAAGTACCTGTTCAGAAAGGA | 1097 |
| 116 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGTAGCGGAACCTACAATGAGCAGTTCTTCGG | |
| | | GCCAGGGACACGGCTCACCGTGCTAG | |
| | Variable-b | DGGITQSPKYLFRKEGQNVTLSCEQNLNHDAMYWYRQDPGQGLRLI | 1098 |
| | | YYSQIVNDFQKGDIAEGYSVSREKKESFPLTVTSAQKNPTAFYLCAS | |
| | | SSGTYNEQFFGPGTRLTVL | |
| | CDR1-b | LNHDA | 216 |
| | CDR2-b | SQIVND | 217 |
| | CDR3-b | CASSSGTYNEQFF | 1099 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVL | 9 |
| TCRA- | a-Variable | GCCCAGAGAGTGACTCAGCCCGAGAAGCTCCTCTCTGTCTTTAAA | 1100 |
| 116 | nucleotide | GGGGCCCCAGTGGAGCTGAAGTGCAACTATTCCTATTCTGGGAGT | |
| | | CCTGAACTCTTCTGGTATGTCCAGTACTCCAGACAACGCCTCCAG | |
| | | TTACTCTTGAGACACATCTCTAGAGAGAGCATCAAAGGCTTCACT | |
| | | GCTGACCTTAACAAAGGCGAGACATCTTTCCACCTGAAGAAACC | |
| | | ATTTGCTCAAGAGGAAGACTCAGCCATGTATTACTGTGCTTCGCC | |
| | | GAGCAAAGCTGCAGGCAACAAGCTAACTTTTGGAGGAGGAACCA | |
| | | GGGTGCTAGTTAAACCAA | |
| | Variable-a | AQRVTQPEKLLSVFKGAPVELKCNYSYSGSPELFWYVQYSRQRLQL | 1101 |
| | | LLRHISRESIKGFTADLNKGETSFHLKKPFAQEEDSAMYYCASPSKA | |
| | | AGNKLTFGGGTRVLVKP | |
| | CDR1-a | YSGSPE | 70 |
| | CDR2-a | HISR | 71 |
| | CDR3-a | CASPSKAAGNKLTF | 1102 |
| | FWR1-a | AQRVTQPEKLLSVFKGAPVELKCNYS | 73 |
| | FWR2-a | LFWYVQYSRQRLQLLLR | 74 |
| | FWR3-a | ESIKGFTADLNKGETSFHLKKPFAQEEDSAMYY | 75 |
| | FWR4-a | GGGTRVLVKP | 1103 |
| TCRB- | b-Variable | GATACTGGAGTCTCCCAGAACCCCAGACACAAGATCACAAAGAG | 1104 |
| 117 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCTTAGAGGCCAGGGCCCCCTCTGGAAACA | |
| | | CCATATATTTTGGAGAGGGAAGTTGGCTCACTGTTGTAG | |
| | Variable-b | DTGVSQNPRHKITKRGQNVTFRCDPISEHNRLYWYRQTLGQGPEFLT | 1105 |
| | | YFQNEAQLEKSRLLSDRFSAERPKGSFSTLEIQRTEQGDSAMYLCASS | |
| | | LEARAPSGNTIYFGEGSWLTVV | |
| | CDR1-b | SEHNR | 442 |
| | CDR2-b | FQNEAQ | 443 |
| | CDR3-b | CASSLEARAPSGNTIYF | 1106 |
| | FWR1-b | DTGVSQNPRHKITKRGQNVTFRCDPI | 445 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GEGSWLTVV | 871 |
| TCRA- | a-Variable | GCTCAGAAGGTAACTCAAGCGCAGACTGAAATTTCTGTGGTGGA | 1107 |
| 117 | nucleotide | GAAGGAGGATGTGACCTTGGACTGTGTGTATGAAACCCGTGATA | |
| | | CTACTTATTACTTATTCTGGTACAAGCAACCACCAAGTGGAGAAT | |
| | | TGGTTTTCCTTATTCGTCGGAACTCTTTTGATGAGCAAAATGAAAT | |
| | | AAGTGGTCGGTATTCTTGGAACTTCCAGAAATCCACCAGTTCCTT | |
| | | CAACTTCACCATCACAGCCTCACAAGTCGTGGACTCAGCAGTATA | |
| | | CTTCTGTGCTCTATGGGAGGGTCAGGGCGGATCTGAAAAGCTGGT | |
| | | CTTTGGAAAGGGAACGAAACTGACAGTAAACCCAT | |
| | Variable-a | AQKVTQAQTEISVVEKEDVTLDCVYETRDTTYYLFWYKQPPSGELV | 1108 |
| | | FLIRRNSFDEQNEISGRYSWNFQKSTSSFNFTITASQVVDSAVYFCAL | |
| | | WEGQGGSEKLVFGKGTKLTVNP | |
| | CDR1-a | TRDTTYY | 48 |
| | CDR2-a | RNSFDEQN | 49 |
| | CDR3-a | CALWEGQGGSEKLVF | 1109 |
| | FWR1-a | AQKVTQAQTEISVVEKEDVTLDCVYE | 51 |
| | FWR2-a | LFWYKQPPSGELVFLIR | 52 |
| | FWR3-a | EISGRYSWNFQKSTSSFNFTITASQVVDSAVYF | 53 |
| | FWR4-a | GKGTKLTVNP | 339 |
| TCRB- | b-Variable | GATACTGGAGTCTCCCAGAACCCCAGACACAAGATCACAAAGAG | 1110 |
| 118 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCACCACACTCCCGGGGGACACTGAAGCTTTCTTTG | |
| | | GACAAGGCACCAGACTCACAGTTGTAG | |
| | Variable-b | DTGVSQNPRHKITKRGQNVTFRCDPISEHNRLYWYRQTLGQGPEFLT | 1111 |
| | | YFQNEAQLEKSRLLSDRFSAERPKGSFSTLEIQRTEQGDSAMYLCAT | |
| | | TLPGDTEAFFGQGTRLTVV | |
| | CDR1-b | SEHNR | 442 |
| | CDR2-b | FQNEAQ | 443 |
| | CDR3-b | CATTLPGDTEAFF | 1112 |
| | FWR1-b | DTGVSQNPRHKITKRGQNVTFRCDPI | 445 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GQGTRLTVV | 246 |
| TCRA- | a-Variable | GCCCAGACAGTCACTCAGTCTCAACCAGAGATGTCTGTGCAGGA | 1113 |
| 118 | nucleotide | GGCAGAGACTGTGACCCTGAGTTGCACATATGACACCAGTGAGA | |
| | | GTAATTATTATTTGTTCTGGTACAAACAGCCTCCCAGCAGGCAGA | |
| | | TGATTCTCGTTATTCGCCAAGAAGCTTATAAGCAACAGAATGCAA | |
| | | CGGAGAATCGTTTCTCTGTGAACTTCCAGAAAGCAGCCAAATCCT | |
| | | TCAGTCTCAAGATCTCAGACTCACAGCTGGGGGACACTGCGATGT | |
| | | ATTTCTGTGCTTTCATGAAGGGGAGAGATGACAAGATCATCTTTG | |
| | | GAAAAGGGACACGACTTCATATTCTCCCCA | |
| | Variable-a | AQTVTQSQPEMSVQEAETVTLSCTYDTSESNYYLFWYKQPPSRQMI | 1114 |
| | | LVIRQEAYKQQNATENRFSVNFQKAAKSFSLKISDSQLGDTAMYFC | |
| | | AFMKGRDDKIIFGKGTRLHILP | |
| | CDR1-a | TSESNYY | 1115 |
| | CDR2-a | QEAYKQQN | 287 |
| | CDR3-a | CAFMKGRDDKIIF | 1116 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDTAMYF | 488 |
| | FWR4-a | GKGTRLHILP | 607 |
| TCRB- | b-Variable | GATGCTGGAGTTATCCAGTCACCCCGGCACGAGGTGACAGAGAT | 1117 |
| 119 | nucleotide | GGGACAAGAAGTGACTCTGAGATGTAAACCAATTTCAGGACACG | |
| | | ACTACCTTTTCTGGTACAGACAGACCATGATGCGGGGACTGGAGT | |
| | | TGCTCATTTACTTTAACAACAACGTTCCGATAGATGATTCAGGGA | |
| | | TGCCCGAGGATCGATTCTCAGCTAAGATGCCTAATGCATCATTCT | |
| | | CCACTCTGAAGATCCAGCCCTCAGAACCCAGGGACTCAGCTGTGT | |
| | | ACTTCTGTGCCAGCAGTTTGGACCGCCATCAGCCCCAGCATTTTG | |
| | | GTGATGGGACTCGACTCTCCATCCTAG | |
| | Variable-b | DAGVIQSPRHEVTEMGQEVTLRCKPISGHDYLFWYRQTMMRGLELL | 1118 |
| | | IYFNNNVPIDDSGMPEDRFSAKMPNASFSTLKIQPSEPRDSAVYFCAS | |
| | | SLDRHQPQHFGDGTRLSIL | |
| | CDR1-b | SGHDY | 468 |
| | CDR2-b | FNNNVP | 469 |
| | CDR3-b | CASSLDRHQPQHF | 1119 |
| | FWR1-b | DAGVIQSPRHEVTEMGQEVTLRCKPI | 471 |
| | FWR2-b | LFWYRQTMMRGLELLIY | 472 |
| | FWR3-b | IDDSGMPEDRFSAKMPNASFSTLKIQPSEPRDSAVYF | 473 |
| | FWR4-b | GDGTRLSIL | 27 |
| TCRA- | a-Variable | AGTCAACAGGGAGAAGAGGATCCTCAGGCCTTGAGCATCCAGGA | 1120 |
| 119 | nucleotide | GGGTGAAAATGCCACCATGAACTGCAGTTACAAAACTAGTATAA | |
| | | ACAATTTACAGTGGTATAGACAAAATTCAGGTAGAGGCCTTGTCC | |
| | | ACCTAATTTTAATACGTTCAAATGAAAGAGAGAAACACAGTGGA | |
| | | AGATTAAGAGTCACGCTTGACACTTCCAAGAAAAGCAGTTCCTTG | |
| | | TTGATCACGGCTTCCCGGGCAGCAGACACTGCTTCTTACTTCTGT | |
| | | GCTACGGCAAATAATGCAGGCAACATGCTCACCTTTGGAGGGGG | |
| | | AACAAGGTTAATGGTCAAACCCC | |
| | Variable-a | SQQGEEDPQALSIQEGENATMNCSYKTSINNLQWYRQNSGRGLVHL | 1121 |
| | | ILIRSNEREKHSGRLRVTLDTSKKSSSLLITASRAADTASYFCATANN | |
| | | AGNMLTFGGGTRLMVKP | |
| | CDR1-a | TSINN | 552 |
| | CDR2-a | IRSNERE | 553 |
| | CDR3-a | CATANNAGNMLTF | 1122 |
| | FWR1-a | SQQGEEDPQALSIQEGENATMNCSYK | 555 |
| | FWR2-a | LQWYRQNSGRGLVHLIL | 556 |
| | FWR3-a | KHSGRLRVTLDTSKKSSSLLITASRAADTASYF | 557 |
| | FWR4-a | GGGTRLMVKP | 141 |
| TCRB- | b-Variable | GACGCTGGAGTCACCCAAAGTCCCACACACCTGATCAAAACGAG | 1123 |
| 120 | nucleotide | AGGACAGCACGTGACTCTGAGATGCTCTCCTATCTCTGGGCACAA | |
| | | GAGTGTGTCCTGGTACCAACAGGTCCTGGGTCAGGGGCCCCAGTT | |
| | | TATCTTTCAGTATTATGAGAAAGAAGAGAGAGGAAGAGGAAACT | |
| | | TCCCTGATCGATTCTCAGCTCGCCAGTTCCCTAACTATAGCTCTGA | |
| | | GCTGAATGTGAACGCCTTGTTGCTGGGGGACTCGGCCCTGTATCT | |
| | | CTGTGCCAGCAGCTTGGAACTAGCGGGAGGGCGTGATACGCAGT | |
| | | ATTTTGGCCCAGGCACCCGGCTGACAGTGCTCG | |
| | Variable-b | DAGVTQSPTHLIKTRGQHVTLRCSPISGHKSVSWYQQVLGQGPQFIF | 1124 |
| | | QYYEKEERGRGNFPDRFSARQFPNYSSELNVNALLLGDSALYLCASS | |
| | | LELAGGRDTQYFGPGTRLTVL | |
| | CDR1-b | SGHKS | 1125 |
| | CDR2-b | YYEKEE | 1126 |
| | CDR3-b | CASSLELAGGRDTQYF | 1127 |
| | FWR1-b | DAGVTQSPTHLIKTRGQHVTLRCSPI | 1128 |
| | FWR2-b | VSWYQQVLGQGPQFIFQ | 1129 |
| | FWR3-b | RGRGNFPDRFSARQFPNYSSELNVNALLLGDSALYL | 1130 |
| | FWR4-b | GPGTRLTVL | 9 |
| TCRA- | a-Variable | GCCCAGTCAGTGACCCAGCCTGACATCCACATCACTGTCTCTGAA | 1131 |
| 120 | nucleotide | GGAGCCTCACTGGAGTTGAGATGTAACTATTCCTATGGGGCAACA | |
| | | CCTTATCTCTTCTGGTATGTCCAGTCCCCCGGCCAAGGCCTCCAGC | |
| | | TGCTCCTGAAGTACTTTTCAGGAGACACTCTGGTTCAAGGCATTA | |
| | | AAGGCTTTGAGGCTGAATTTAAGAGGAGTCAATCTTCCTTCAACC | |
| | | TGAGGAAACCCTCTGTGCATTGGAGTGATGCTGCTGAGTACTTCT | |
| | | GTGCTGTGGGTGCCGGAACCGGCACTGCCAGTAAACTCACCTTTG | |
| | | GGACTGGAACAAGACTTCAGGTCACGCTCG | |
| | Variable-a | AQSVTQPDIHITVSEGASLELRCNYSYGATPYLFWYVQSPGQGLQLL | 1132 |
| | | LKYFSGDTL VQGIKGFEAEFKRSQSSFNLRKPSVHWSDAAEYFCAVG | |
| | | AGTGTASKLTFGTGTRLQVTL | |
| | CDR1-a | YGATPY | 886 |
| | CDR2-a | YFSGDTLV | 887 |
| | CDR3-a | CAVGAGTGTASKLTF | 1133 |
| | FWR1-a | AQSVTQPDIHITVSEGASLELRCNYS | 889 |
| | FWR2-a | LFWYVQSPGQGLQLLLK | 890 |
| | FWR3-a | QGIKGFEAEFKRSQSSFNLRKPSVHWSDAAEYF | 891 |
| | FWR4-a | GTGTRLQVTL | 1134 |
| TCRB- | b-Variable | GATTCTGGAGTCACACAAACCCCAAAGCACCTGATCACAGCAAC | 1135 |
| 121 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCGAGGGGACAGTCCTTGACGAGCAGTACTTCG | |
| | | GGCCGGGCACCAGGCTCACGGTCACAG | |
| | Variable-b | DSGVTQTPKHLITATGQRVTLRCSPRSGDLSVYWYQQSLDQGLQFLI | 1136 |
| | | QYYNGEERAKGNILERFSAQQFPDLHSELNLSSLELGDSALYFCASSE | |
| | | GTVLDEQYFGPGTRLTVT | |
| | CDR1-b | SGDLS | 383 |
| | CDR2-b | YYNGEE | 384 |
| | CDR3-b | CASSEGTVLDEQYF | 1137 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GPGTRLTVT | 85 |
| TCRA- | a-Variable | GCTCAGAAGGTAACTCAAGCGCAGACTGAAATTTCTGTGGTGGA | 1138 |
| 121 | nucleotide | GAAGGAGGATGTGACCTTGGACTGTGTGTATGAAACCCGTGATA | |
| | | CTACTTATTACTTATTCTGGTACAAGCAACCACCAAGTGGAGAAT | |
| | | TGGTTTTCCTTATTCGTCGGAACTCTTTTGATGAGCAAAATGAAAT | |
| | | AAGTGGTCGGTATTCTTGGAACTTCCAGAAATCCACCAGTTCCTT | |
| | | CAACTTCACCATCACAGCCTCACAAGTCGTGGACTCAGCAGTATA | |
| | | CTTCTGTGCTCTGAGTGAAGGATCTAACTTTGGAAATGAGAAATT | |
| | | AACCTTTGGGACTGGAACAAGACTCACCATCATACCCA | |
| | Variable-a | AQKVTQAQTEISVVEKEDVTLDCVYETRDTTYYLFWYKQPPSGELV | 1139 |
| | | FLIRRNSFDEQNEISGRYSWNFQKSTSSFNFTITASQVVDSAVYFCAL | |
| | | SEGSNFGNEKLTFGTGTRLTIIP | |
| | CDR1-a | TRDTTYY | 48 |
| | CDR2-a | RNSFDEQN | 49 |
| | CDR3-a | CALSEGSNFGNEKLTF | 1140 |
| | FWR1-a | AQKVTQAQTEISVVEKEDVTLDCVYE | 51 |
| | FWR2-a | LFWYKQPPSGEL VFLIR | 52 |
| | FWR3-a | EISGRYSWNFQKSTSSFNFTITASQVVDSAVYF | 53 |
| | FWR4-a | GTGTRLTIIP | 292 |
| TCRB- | b-Variable | GAAGCCCAAGTGACCCAGAACCCAAGATACCTCATCACAGTGAC | 1141 |
| 122 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTTTGGGGTCGTCTACGCAGTATTTTGGCCCAG | |
| | | GCACCCGGCTGACAGTGCTCG | |
| | Variable-b | EAQVTQNPRYLITVTGKKLTVTCSQNMNHEYMSWYRQDPGLGLRQ | 1142 |
| | | IYYSMNVEVTDKGDVPEGYKVSRKEKRNFPLILESPSPNQTSLYFCA | |
| | | SSLGSSTQYFGPGTRLTVL | |
| | CDR1-b | MNHEY | 252 |
| | CDR2-b | SMNVEV | 366 |
| | CDR3-b | CASSLGSSTQYF | 1143 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GPGTRLTVL | 9 |
| TCRA- | a-Variable | AAACAGGAGGTGACGCAGATTCCTGCAGCTCTGAGTGTCCCAGA | 1144 |
| 122 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGCGGGGGGGAATTATGGAGGAAGCCAAGGAAATCTCATCT | |
| | | TTGGAAAAGGCACTAAACTCTCTGTTAAACCAA | |
| | Variable-a | KQEVTQIPAALSVPEGENLVLNCSFTDSAIYNLQWFRQDPGKGLTSL | 1145 |
| | | LLIQSSQREQTSGRLNASLDKSSGRSTLYIAASQPGDSATYLCAAGG | |
| | | NYGGSQGNLIFGKGTKLSVKP | |
| | CDR1-a | DSAIYN | 118 |
| | CDR2-a | IQSSQRE | 119 |
| | CDR3-a | CAAGGNYGGSQGNLIF | 1146 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GKGTKLSVKP | 124 |
| TCRB- | b-Variable | AAGGCTGGAGTCACTCAAACTCCAAGATATCTGATCAAAACGAG | 1147 |
| 123 | nucleotide | AGGACAGCAAGTGACACTGAGCTGCTCCCCTATCTCTGGGCATAG | |
| | | GAGTGTATCCTGGTACCAACAGACCCCAGGACAGGGCCTTCAGTT | |
| | | CCTCTTTGAATACTTCAGTGAGACACAGAGAAACAAAGGAAACT | |
| | | TCCCTGGTCGATTCTCAGGGCGCCAGTTCTCTAACTCTCGCTCTGA | |
| | | GATGAATGTGAGCACCTTGGAGCTGGGGGACTCGGCCCTTTATCT | |
| | | TTGCGCCAGCAGCTTGGAGCCTAGCGGGGGGCGGAGGTGGGACA | |
| | | ATGAGCAGTTCTTCGGGCCAGGGACACGGCTCACCGTGCTAG | |
| | Variable-b | KAGVTQTPRYLIKTRGQQVTLSCSPISGHRSVSWYQQTPGQGLQFLF | 1148 |
| | | EYFSETQRNKGNFPGRFSGRQFSNSRSEMNVSTLELGDSALYLCASS | |
| | | LEPSGGRRWDNEQFFGPGTRLTVL | |
| | CDR1-b | SGHRS | 514 |
| | CDR2-b | YFSETQ | 515 |
| | CDR3-b | CASSLEPSGGRRWDNEQFF | 1149 |
| | FWR1-b | KAGVTQTPRYLIKTRGQQVTLSCSPI | 517 |
| | FWR2-b | VSWYQQTPGQGLQFLFE | 518 |
| | FWR3-b | RNKGNFPGRFSGRQFSNSRSEMNVSTLELGDSALYL | 519 |
| | FWR4-b | GPGTRLTVL | 9 |
| TCRA- | a-Variable | GGAAATTCAGTGACCCAGATGGAAGGGCCAGTGACTCTCTCAGA | 1150 |
| 123 | nucleotide | AGAGGCCTTCCTGACTATAAACTGCACGTACACAGCCACAGGAT | |
| | | ACCCTTCCCTTTTCTGGTATGTCCAATATCCTGGAGAAGGTCTACA | |
| | | GCTCCTCCTGAAAGCCACGAAGGCTGATGACAAGGGAAGCAACA | |
| | | AAGGTTTTGAAGCCACATACCGTAAAGAAACCACTTCTTTCCACT | |
| | | TGGAGAAAGGCTCAGTTCAAGTGTCAGACTCAGCGGTGTACTTCT | |
| | | GTGCTCTGACTGGGCCCCAAGCTAATGCTGGTGGTACTAGCTATG | |
| | | GAAAGCTGACATTTGGACAAGGGACCATCTTGACTGTCCATCCAA | |
| | Variable-a | GNSVTQMEGPVTLSEEAFLTINCTYTATGYPSLFWYVQYPGEGLQLL | 1151 |
| | | LKATKADDKGSNKGFEATYRKETTSFHLEKGSVQVSDSAVYFCALT | |
| | | GPQANAGGTSYGKLTFGQGTILTVHP | |
| | CDR1-a | ATGYPS | 106 |
| | CDR2-a | ATKADDK | 107 |
| | CDR3-a | CALTGPQANAGGTSYGKLTF | 1152 |
| | FWR1-a | GNSVTQMEGPVTLSEEAFLTINCTYT | 724 |
| | FWR2-a | LFWYVQYPGEGLQLLLK | 110 |
| | FWR3-a | GSNKGFEATYRKETTSFHLEKGSVQVSDSAVYF | 111 |
| | FWR4-a | GQGTILTVHP | 76 |
| TCRB- | b-Variable | AAGGCTGGAGTCACTCAAACTCCAAGATATCTGATCAAAACGAG | 1153 |
| 124 | nucleotide | AGGACAGCAAGTGACACTGAGCTGCTCCCCTATCTCTGGGCATAG | |
| | | GAGTGTATCCTGGTACCAACAGACCCCAGGACAGGGCCTTCAGTT | |
| | | CCTCTTTGAATACTTCAGTGAGACACAGAGAAACAAAGGAAACT | |
| | | TCCCTGGTCGATTCTCAGGGCGCCAGTTCTCTAACTCTCGCTCTGA | |
| | | GATGAATGTGAGCACCTTGGAGCTGGGGGACTCGGCCCTTTATCT | |
| | | TTGCGCCAGCAGCGAGGATACCTATGGCTACACCTTCGGTTCGGG | |
| | | GACCAGGTTAACCGTTGTAG | |
| | Variable-b | KAGVTQTPRYLIKTRGQQVTLSCSPISGHRSVSWYQQTPGQGLQFLF | 1154 |
| | | EYFSETQRNKGNFPGRFSGRQFSNSRSEMNVSTLELGDSALYLCASS | |
| | | EDTYGYTFGSGTRLTVV | |
| | CDR1-b | SGHRS | 514 |
| | CDR2-b | YFSETQ | 515 |
| | CDR3-b | CASSEDTYGYTF | 1155 |
| | FWR1-b | KAGVTQTPRYLIKTRGQQVTLSCSPI | 517 |
| | FWR2-b | VSWYQQTPGQGLQFLFE | 518 |
| | FWR3-b | RNKGNFPGRFSGRQFSNSRSEMNVSTLELGDSALYL | 519 |
| | FWR4-b | GSGTRLTVV | 103 |
| TCRA- | a-Variable | AAACAGGAGGTGACGCAGATTCCTGCAGCTCTGAGTGTCCCAGA | 1156 |
| 124 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGATCGTCACCGGTAACCAGTTCTATTTTGGGACAGGGAC | |
| | | AAGTTTGACGGTCATTCCAA | |
| | Variable-a | KQEVTQIPAALSVPEGENLVLNCSFTDSAIYNLQWFRQDPGKGLTSL | 1157 |
| | | LLIQSSQREQTSGRLNASLDKSSGRSTLYIAASQPGDSATYLCAVIVT | |
| | | GNQFYFGTGTSLTVIP | |
| | CDR1-a | DSAIYN | 118 |
| | CDR2-a | IQSSQRE | 119 |
| | CDR3-a | CAVIVTGNQFYF | 1158 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GTGTSLTVIP | 94 |
| TCRB- | b-Variable | GACGCTGGAGTCACCCAAAGTCCCACACACCTGATCAAAACGAG | 1159 |
| 125 | nucleotide | AGGACAGCAAGTGACTCTGAGATGCTCTCCTAAGTCTGGGCATGA | |
| | | CACTGTGTCCTGGTACCAACAGGCCCTGGGTCAGGGGCCCCAGTT | |
| | | TATCTTTCAGTATTATGAGGAGGAAGAGAGACAGAGAGGCAACT | |
| | | TCCCTGATCGATTCTCAGGTCACCAGTTCCCTAACTATAGCTCTGA | |
| | | GCTGAATGTGAACGCCTTGTTGCTGGGGGACTCGGCCCTCTATCT | |
| | | CTGTGCCAGCAGCTTCCTAACTGACTCCTACAATGAGCAGTTCTT | |
| | | CGGGCCAGGGACACGGCTCACCGTGCTAG | |
| | Variable-b | DAGVTQSPTHLIKTRGQQVTLRCSPKSGHDTVSWYQQALGQGPQFIF | 1160 |
| | | QYYEEEERQRGNFPDRFSGHQFPNYSSELNVNALLLGDSALYLCASS | |
| | | FLTDSYNEQFFGPGTRLTVL | |
| | CDR1-b | SGHDT | 331 |
| | CDR2-b | YYEEEE | 332 |
| | CDR3-b | CASSFLTDSYNEQFF | 1161 |
| | FWR1-b | DAGVTQSPTHLIKTRGQQVTLRCSPK | 334 |
| | FWR2-b | VSWYQQALGQGPQFIFQ | 131 |
| | FWR3-b | RQRGNFPDRFSGHQFPNYSSELNVNALLLGDSALYL | 335 |
| | FWR4-b | GPGTRLTVL | 9 |
| TCRA- | a-Variable | AGTCAACAGGGAGAAGAGGATCCTCAGGCCTTGAGCATCCAGGA | 1162 |
| 125 | nucleotide | GGGTGAAAATGCCACCATGAACTGCAGTTACAAAACTAGTATAA | |
| | | ACAATTTACAGTGGTATAGACAAAATTCAGGTAGAGGCCTTGTCC | |
| | | ACCTAATTTTAATACGTTCAAATGAAAGAGAGAAACACAGTGGA | |
| | | AGATTAAGAGTCACGCTTGACACTTCCAAGAAAAGCAGTTCCTTG | |
| | | TTGATCACGGCTTCCCGGGCAGCAGACACTGCTTCTTACTTCTGT | |
| | | GCTACGGACTACCGGACAGGAGGAAGCTACATACCTACATTTGG | |
| | | AAGAGGAACCAGCCTTATTGTTCATCCGT | |
| | Variable-a | SQQGEEDPQALSIQEGENATMNCSYKTSINNLQWYRQNSGRGLVHL | 1163 |
| | | ILIRSNEREKHSGRLRVTLDTSKKSSSLLITASRAADTASYFCATDYR | |
| | | TGGSYIPTFGRGTSLIVHP | |
| | CDR1-a | TSINN | 552 |
| | CDR2-a | IRSNERE | 553 |
| | CDR3-a | CATDYRTGGSYIPTF | 1164 |
| | FWR1-a | SQQGEEDPQALSIQEGENATMNCSYK | 555 |
| | FWR2-a | LQWYRQNSGRGLVHLIL | 556 |
| | FWR3-a | KHSGRLRVTLDTSKKSSSLLITASRAADTASYF | 557 |
| | FWR4-a | GRGTSLIVHP | 600 |
| TCRB- | b-Variable | GAAGCTGAAGTTGCCCAGTCCCCCAGATATAAGATTACAGAGAA | 1165 |
| 126 | nucleotide | AAGCCAGGCTGTGGCTTTTTGGTGTGATCCTATTTCTGGCCATGCT | |
| | | ACCCTTTACTGGTACCGGCAGATCCTGGGACAGGGCCCGGAGCTT | |
| | | CTGGTTCAATTTCAGGATGAGAGTGTAGTAGATGATTCACAGTTG | |
| | | CCTAAGGATCGATTTTCTGCAGAGAGGCTCAAAGGAGTAGACTCC | |
| | | ACTCTCAAGATCCAGCCTGCAGAGCTTGGGGACTCGGCCATGTAT | |
| | | CTCTGTGCCAGCAGCTACCTAGCGGGAGGGTTTAGGGATGAGCA | |
| | | GTTCTTCGGGCCAGGGACACGGCTCACCGTGCTAG | |
| | Variable-b | EAEVAQSPRYKITEKSQAVAFWCDPISGHATLYWYRQILGQGPELLV | 1166 |
| | | QFQDESVVDDSQLPKDRFSAERLKGVDSTLKIQPAELGDSAMYLCA | |
| | | SSYLAGGFRDEQFFGPGTRLTVL | |
| | CDR1-b | SGHAT | 894 |
| | CDR2-b | FQDESV | 1167 |
| | CDR3-b | CASSYLAGGFRDEQFF | 1168 |
| | FWR1-b | EAEVAQSPRYKITEKSQAVAFWCDPI | 1169 |
| | FWR2-b | LYWYRQILGQGPELLVQ | 1170 |
| | FWR3-b | VDDSQLPKDRFSAERLKGVDSTLKIQPAELGDSAMYL | 1171 |
| | FWR4-b | GPGTRLTVL | 9 |
| TCRA- | a-Variable | GGAAATTCAGTGACCCAGATGGAAGGGCCAGTGACTCTCTCAGA | 1172 |
| 126 | nucleotide | AGAGGCCTTCCTGACTATAAACTGCACGTACACAGCCACAGGAT | |
| | | ACCCTTCCCTTTTCTGGTATGTCCAATATCCTGGAGAAGGTCTACA | |
| | | GCTCCTCCTGAAAGCCACGAAGGCTGATGACAAGGGAAGCAACA | |
| | | AAGGTTTTGAAGCCACATACCGTAAAGAAACCACTTCTTTCCACT | |
| | | TGGAGAAAGGCTCAGTTCAAGTGTCAGACTCAGCGGTGTACTTCT | |
| | | GTGCTCTGAGTGGCAGGGAATATGGAAACAAGCTGGTCTTTGGC | |
| | | GCAGGAACCATTCTGAGAGTCAAGTCCT | |
| | Variable-a | GNSVTQMEGPVTLSEEAFLTINCTYTATGYPSLFWYVQYPGEGLQLL | 1173 |
| | | LKATKADDKGSNKGFEATYRKETTSFHLEKGSVQVSDSAVYFCALS | |
| | | GREYGNKLVFGAGTILRVKS | |
| | CDR1-a | ATGYPS | 106 |
| | CDR2-a | ATKADDK | 107 |
| | CDR3-a | CALSGREYGNKLVF | 1174 |
| | FWR1-a | GNSVTQMEGPVTLSEEAFLTINCTYT | 724 |
| | FWR2-a | LFWYVQYPGEGLQLLLK | 110 |
| | FWR3-a | GSNKGFEATYRKETTSFHLEKGSVQVSDSAVYF | 111 |
| | FWR4-a | GAGTILRVKS | 18 |
| TCRB- | b-Variable | GATTCTGGAGTCACACAAACCCCAAAGCACCTGATCACAGCAAC | 1175 |
| 127 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCCCCCCGGACAGCTATGGCTACACCTTCGGTTC | |
| | | GGGGACCAGGTTAACCGTTGTAG | |
| | Variable-b | DSGVTQTPKHLITATGQRVTLRCSPRSGDLSVYWYQQSLDQGLQFLI | 1176 |
| | | QYYNGEERAKGNILERFSAQQFPDLHSELNLSSLELGDSALYFCASSP | |
| | | PDSYGYTFGSGTRLTVV | |
| | CDR1-b | SGDLS | 383 |
| | CDR2-b | YYNGEE | 384 |
| | CDR3-b | CASSPPDSYGYTF | 1177 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GSGTRLTVV | 103 |
| TCRA- | a-Variable | GGACAAAACATTGACCAGCCCACTGAGATGACAGCTACGGAAGG | 1178 |
| 127 | nucleotide | TGCCATTGTCCAGATCAACTGCACGTACCAGACATCTGGGTTCAA | |
| | | CGGGCTGTTCTGGTACCAGCAACATGCTGGCGAAGCACCTACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTG | |
| | | AGGGGCGCAACAAAGCTCAGCTTTGGAGCCGGAACCACAGTAAC | |
| | | TGTAAGAGCAA | |
| | Variable-a | GQNIDQPTEMTATEGAIVQINCTYQTSGFNGLFWYQQHAGEAPTFLS | 1179 |
| | | YNVLDGLEEKGRESSFLSRSKGYSYLLLKELQMKDSASYLCAVRGA | |
| | | TKLSFGAGTTVTVRA | |
| | CDR1-a | TSGFNG | 450 |
| | CDR2-a | NVLDGL | 451 |
| | CDR3-a | CAVRGATKLSF | 1180 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GAGTTVTVRA | 1181 |
| TCRB- | b-Variable | AATGCTGGTGTCACTCAGACCCCAAAATTCCGGGTCCTGAAGACA | 1182 |
| 128 | nucleotide | GGACAGAGCATGACACTGCTGTGTGCCCAGGATATGAACCATGA | |
| | | ATACATGTACTGGTATCGACAAGACCCAGGCATGGGGCTGAGGC | |
| | | TGATTCATTACTCAGTTGGTGAGGGTACAACTGCCAAAGGAGAG | |
| | | GTCCCTGATGGCTACAATGTCTCCAGATTAAAAAAACAGAATTTC | |
| | | CTGCTGGGGTTGGAGTCGGCTGCTCCCTCCCAAACATCTGTGTAC | |
| | | TTCTGTGCCAGCAGTCCCGTCGGTTGGCGCGAGCAGTTCTTCGGG | |
| | | CCAGGGACACGGCTCACCGTGCTAG | |
| | Variable-b | NAGVTQTPKFRVLKTGQSMTLLCAQDMNHEYMYWYRQDPGMGLR | 1183 |
| | | LIHYSVGEGTTAKGEVPDGYNVSRLKKQNFLLGLESAAPSQTSVYFC | |
| | | ASSPVGWREQFFGPGTRLTVL | |
| | CDR1-b | MNHEY | 252 |
| | CDR2-b | SVGEGT | 1046 |
| | CDR3-b | CASSPVGWREQFF | 1184 |
| | FWR1-b | NAGVTQTPKFRVLKTGQSMTLLCAQD | 1048 |
| | FWR2-b | MYWYRQDPGMGLRLIHY | 1049 |
| | FWR3-b | TAKGEVPDGYNVSRLKKQNFLLGLESAAPSQTSVYF | 1050 |
| | FWR4-b | GPGTRLTVL | 9 |
| TCRA- | a-Variable | GGAGAGGATGTGGAGCAGAGTCTTTTCCTGAGTGTCCGAGAGGG | 1185 |
| 128 | nucleotide | AGACAGCTCCGTTATAAACTGCACTTACACAGACAGCTCCTCCAC | |
| | | CTACTTATACTGGTATAAGCAAGAACCTGGAGCAGGTCTCCAGTT | |
| | | GCTGACGTATATTTTTTCAAATATGGACATGAAACAAGACCAAAG | |
| | | ACTCACTGTTCTATTGAATAAAAAGGATAAACATCTGTCTCTGCG | |
| | | CATTGCAGACACCCAGACTGGGGACTCAGCTATCTACTTCTGTGC | |
| | | AGAGAGCCCGTCAGGAGGAAGCTACATACCTACATTTGGAAGAG | |
| | | GAACCAGCCTTATTGTTCATCCGT | |
| | Variable-a | GEDVEQSLFLSVREGDSSVINCTYTDSSSTYLYWYKQEPGAGLQLLT | 1186 |
| | | YIFSNMDMKQDQRLTVLLNKKDKHLSLRIADTQTGDSAIYFCAESPS | |
| | | GGSYIPTFGRGTSLIVHP | |
| | CDR1-a | DSSSTY | 314 |
| | CDR2-a | IFSNMDM | 315 |
| | CDR3-a | CAESPSGGSYIPTF | 1187 |
| | FWR1-a | GEDVEQSLFLSVREGDSSVINCTYT | 317 |
| | FWR2-a | LYWYKQEPGAGLQLLTY | 318 |
| | FWR3-a | KQDQRLTVLLNKKDKHLSLRIADTQTGDSAIYF | 319 |
| | FWR4-a | GRGTSLIVHP | 600 |
| TCRB- | b-Variable | GGTGCTGTCGTCTCTCAACATCCGAGCAGGGTTATCTGTAAGAGT | 1188 |
| 129 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGGCCCCCAGGGGGGGGCTACACCT | |
| | | TCGGTTCGGGGACCAGGTTAACCGTTGTAG | |
| | Variable-b | GAVVSQHPSRVICKSGTSVKIECRSLDFQATTMFWYRQFPKQSLML | 1189 |
| | | MATSNEGSKATYEQGVEKDKFLINHASLTLSTLTVTSAHPEDSSFYIC | |
| | | SARPPGGGYTFGSGTRLTVV | |
| | CDR1-b | DFQATT | 79 |
| | CDR2-b | SNEGSKA | 80 |
| | CDR3-b | CSARPPGGGYTF | 1190 |
| | FWR1-b | GAVVSQHPSRVICKSGTSVKIECRSL | 1063 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GSGTRLTVV | 103 |
| TCRA- | a-Variable | ATACTGAACGTGGAACAAAGTCCTCAGTCACTGCATGTTCAGGAG | 1191 |
| 129 | nucleotide | GGAGACAGCACCAATTTCACCTGCAGCTTCCCTTCCAGCAATTTT | |
| | | TATGCCTTACACTGGTACAGATGGGAAACTGCAAAAAGCCCCGA | |
| | | GGCCTTGTTTGTAATGACTTTAAATGGGGATGAAAAGAAGAAAG | |
| | | GACGAATAAGTGCCACTCTTAATACCAAGGAGGGTTACAGCTATT | |
| | | TGTACATCAAAGGATCCCAGCCTGAAGACTCAGCCACATACCTCT | |
| | | GTGCCTTCGATTCAGGATACAGCACCCTCACCTTTGGGAAGGGGA | |
| | | CTATGCTTCTAGTCTCTCCAG | |
| | Variable-a | ILNVEQSPQSLHVQEGDSTNFTCSFPSSNFYALHWYRWETAKSPEAL | 1192 |
| | | FVMTLNGDEKKKGRISATLNTKEGYSYLYIKGSQPEDSATYLCAFDS | |
| | | GYSTLTFGKGTMLLVSP | |
| | CDR1-a | SSNFYA | 762 |
| | CDR2-a | MTLNGDE | 763 |
| | CDR3-a | CAFDSGYSTLTF | 1193 |
| | FWR1-a | ILNVEQSPQSLHVQEGDSTNFTCSFP | 765 |
| | FWR2-a | LHWYRWETAKSPEALFV | 766 |
| | FWR3-a | KKKGRISATLNTKEGYSYLYIKGSQPEDSATYL | 767 |
| | FWR4-a | GKGTMLLVSP | 320 |
| TCRB- | b-Variable | GATGCTGGAGTTATCCAGTCACCCCGGCACGAGGTGACAGAGAT | 1194 |
| 130 | nucleotide | GGGACAAGAAGTGACTCTGAGATGTAAACCAATTTCAGGACACG | |
| | | ACTACCTTTTCTGGTACAGACAGACCATGATGCGGGGACTGGAGT | |
| | | TGCTCATTTACTTTAACAACAACGTTCCGATAGATGATTCAGGGA | |
| | | TGCCCGAGGATCGATTCTCAGCTAAGATGCCTAATGCATCATTCT | |
| | | CCACTCTGAAGATCCAGCCCTCAGAACCCAGGGACTCAGCTGTGT | |
| | | ACTTCTGTGCCAGCAGTTTCCAGACTGTCGGGTCAGGGTCCTACG | |
| | | AGCAGTACTTCGGGCCGGGCACCAGGCTCACGGTCACAG | |
| | Variable-b | DAGVIQSPRHEVTEMGQEVTLRCKPISGHDYLFWYRQTMMRGLELL | 1195 |
| | | IYFNNNVPIDDSGMPEDRFSAKMPNASFSTLKIQPSEPRDSAVYFCAS | |
| | | SFQTVGSGSYEQYFGPGTRLTVT | |
| | CDR1-b | SGHDY | 468 |
| | CDR2-b | FNNNVP | 469 |
| | CDR3-b | CASSFQTVGSGSYEQYF | 1196 |
| | FWR1-b | DAGVIQSPRHEVTEMGQEVTLRCKPI | 471 |
| | FWR2-b | LFWYRQTMMRGLELLIY | 472 |
| | FWR3-b | IDDSGMPEDRFSAKMPNASFSTLKIQPSEPRDSAVYF | 473 |
| | FWR4-b | GPGTRLTVT | 85 |
| TCRA- | a-Variable | GCTCAGAAGGTAACTCAAGCGCAGACTGAAATTTCTGTGGTGGA | 1197 |
| 130 | nucleotide | GAAGGAGGATGTGACCTTGGACTGTGTGTATGAAACCCGTGATA | |
| | | CTACTTATTACTTATTCTGGTACAAGCAACCACCAAGTGGAGAAT | |
| | | TGGTTTTCCTTATTCGTCGGAACTCTTTTGATGAGCAAAATGAAAT | |
| | | AAGTGGTCGGTATTCTTGGAACTTCCAGAAATCCACCAGTTCCTT | |
| | | CAACTTCACCATCACAGCCTCACAAGTCGTGGACTCAGCAGTATA | |
| | | CTTCTGTGCTCTGAGTGAGGTCTCCTCTGGGGCTGGGAGTTACCA | |
| | | ACTCACTTTCGGGAAGGGGACCAAACTCTCGGTCATACCAA | |
| | Variable-a | AQKVTQAQTEISVVEKEDVTLDCVYETRDTTYYLFWYKQPPSGELV | 1198 |
| | | FLIRRNSFDEQNEISGRYSWNFQKSTSSFNFTITASQVVDSAVYFCAL | |
| | | SEVSSGAGSYQLTFGKGTKLSVIP | |
| | CDR1-a | TRDTTYY | 48 |
| | CDR2-a | RNSFDEQN | 49 |
| | CDR3-a | CALSEVSSGAGSYQLTF | 1199 |
| | FWR1-a | AQKVTQAQTEISVVEKEDVTLDCVYE | 51 |
| | FWR2-a | LFWYKQPPSGEL VFLIR | 52 |
| | FWR3-a | EISGRYSWNFQKSTSSFNFTITASQVVDSAVYF | 53 |
| | FWR4-a | GKGTKLSVIP | 489 |
| TCRB- | b-Variable | AATGCTGGTGTCACTCAGACCCCAAAATTCCAGGTCCTGAAGACA | 1200 |
| 131 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTTACGACGGGGAGAACACTGAAGCTTTCTTTGG | |
| | | ACAAGGCACCAGACTCACAGTTGTAG | |
| | Variable-b | NAGVTQTPKFQVLKTGQSMTLQCAQDMNHEYMSWYRQDPGMGLR | 1201 |
| | | LIHYSVGAGITDQGEVPNGYNVSRSTTEDFPLRLLSAAPSQTSVYFCA | |
| | | SSYDGENTEAFFGQGTRLTVV | |
| | CDR1-b | MNHEY | 252 |
| | CDR2-b | SVGAGI | 253 |
| | CDR3-b | CASSYDGENTEAFF | 1202 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GQGTRLTVV | 246 |
| TCRA- | a-Variable | GGAGAGAATGTGGAGCAGCATCCTTCAACCCTGAGTGTCCAGGA | 1203 |
| 131 | nucleotide | GGGAGACAGCGCTGTTATCAAGTGTACTTATTCAGACAGTGCCTC | |
| | | AAACTACTTCCCTTGGTATAAGCAAGAACTTGGAAAAGGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCAAGTACACCGGGTGAGAAATTAACCTTTGGGACTGGA | |
| | | ACAAGACTCACCATCATACCCA | |
| | Variable-a | GENVEQHPSTLSVQEGDSAVIKCTYSDSASNYFPWYKQELGKGPQLI | 1204 |
| | | IDIRSNVGEKKDQRIA VTLNKTAKHFSLHITETQPEDSAVYFCAASTP | |
| | | GEKLTFGTGTRLTIIP | |
| | CDR1-a | DSASNY | 207 |
| | CDR2-a | IRSNVGE | 208 |
| | CDR3-a | CAASTPGEKLTF | 1205 |
| | FWR1-a | GENVEQHPSTLSVQEGDSAVIKCTYS | 210 |
| | FWR2-a | FPWYKQELGKGPQLIID | 374 |
| | FWR3-a | KKDQRIAVTLNKTAKHFSLHITETQPEDSAVYF | 212 |
| | FWR4-a | GTGTRLTIIP | 292 |
| TCRB- | b-Variable | AATGCTGGTGTCACTCAGACCCCAAAATTCCAGGTCCTGAAGACA | 1206 |
| 132 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAAACCGACTAGCGGGAGCGTGAATGAGCAGTTCT | |
| | | TCGGGCCAGGGACACGGCTCACCGTGCTAG | |
| | Variable-b | NAGVTQTPKFQVLKTGQSMTLQCAQDMNHEYMSWYRQDPGMGLR | 1207 |
| | | LIHYSVGAGITDQGEVPNGYNVSRSTTEDFPLRLLSAAPSQTSVYFCA | |
| | | SKPTSGSVNEQFFGPGTRLTVL | |
| | CDR1-b | MNHEY | 252 |
| | CDR2-b | SVGAGI | 253 |
| | CDR3-b | CASKPTSGSVNEQFF | 1208 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GPGTRLTVL | 9 |
| TCRA- | a-Variable | AAACAGGAGGTGACGCAGATTCCTGCAGCTCTGAGTGTCCCAGA | 1209 |
| 132 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGGGTAATACTGGAGGCTTCAAAACTATCTTTGGAGCAGG | |
| | | AACAAGACTATTTGTTAAAGCAA | |
| | Variable-a | KQEVTQIPAALSVPEGENLVLNCSFTDSAIYNLQWFRQDPGKGLTSL | 1210 |
| | | LLIQSSQREQTSGRLNASLDKSSGRSTLYIAASQPGDSATYLCAVGNT | |
| | | GGFKTIFGAGTRLFVKA | |
| | CDR1-a | DSAIYN | 118 |
| | CDR2-a | IQSSQRE | 119 |
| | CDR3-a | CAVGNTGGFKTIF | 1211 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GAGTRLFVKA | 112 |
| TCRB- | b-Variable | AGTGCTGTCATCTCTCAAAAGCCAAGCAGGGATATCTGTCAACGT | 1212 |
| 133 | nucleotide | GGAACCTCCCTGACGATCCAGTGTCAAGTCGATAGCCAAGTCACC | |
| | | ATGATGTTCTGGTACCGTCAGCAACCTGGACAGAGCCTGACACTG | |
| | | ATCGCAACTGCAAATCAGGGCTCTGAGGCCACATATGAGAGTGG | |
| | | ATTTGTCATTGACAAGTTTCCCATCAGCCGCCCAAACCTAACATT | |
| | | CTCAACTCTGACTGTGAGCAACATGAGCCCTGAAGACAGCAGCA | |
| | | TATATCTCTGCAGCGTTGAGGGATCACTGGCCCAGTACTTCGGGC | |
| | | CAGGCACGCGGCTCCTGGTGCTCG | |
| | Variable-b | SAVISQKPSRDICQRGTSLTIQCQVDSQVTMMFWYRQQPGQSLTLIA | 1213 |
| | | TANQGSEATYESGFVIDKFPISRPNLTFSTLTVSNMSPEDSSIYLCSVE | |
| | | GSLAQYFGPGTRLLVL | |
| | CDR1-b | SQVTM | 97 |
| | CDR2-b | ANQGSEA | 98 |
| | CDR3-b | CSVEGSLAQYF | 1214 |
| | FWR1-b | SAVISQKPSRDICQRGTSLTIQCQVD | 100 |
| | FWR2-b | MFWYRQQPGQSLTLIAT | 101 |
| | FWR3-b | TYESGFVIDKFPISRPNLTFSTLTVSNMSPEDSSIYL | 102 |
| | FWR4-b | GPGTRLLVL | 67 |
| TCRA- | a-Variable | GAGCTGAAAGTGGAACAAAACCCTCTGTTCCTGAGCATGCAGGA | 1215 |
| 133 | nucleotide | GGGAAAAAACTATACCATCTACTGCAATTATTCAACCACTTCAGA | |
| | | CAGACTGTATTGGTACAGGCAGGATCCTGGGAAAAGTCTGGAAT | |
| | | CTCTGTTTGTGTTGCTATCAAATGGAGCAGTGAAGCAGGAGGGAC | |
| | | GATTAATGGCCTCACTTGATACCAAAGCCCGTCTCAGCACCCTCC | |
| | | ACATCACAGCTGCCGTGCATGACCTCTCTGCCACCTACTTCTGTG | |
| | | CCGTGGACAGGGACCAAGGAAATCTCATCTTTGGAAAAGGCACT | |
| | | AAACTCTCTGTTAAACCAA | |
| | Variable-a | ELKVEQNPLFLSMQEGKNYTIYCNYSTTSDRLYWYRQDPGKSLESLF | 1216 |
| | | VLLSNGAVKQEGRLMASLDTKARLSTLHITAAVHDLSATYFCAVDR | |
| | | DQGNLIFGKGTKLSVKP | |
| | CDR1-a | TTSDR | 973 |
| | CDR2-a | LLSNGAV | 974 |
| | CDR3-a | CAVDRDQGNLIF | 1217 |
| | FWR1-a | ELKVEQNPLFLSMQEGKNYTIYCNYS | 976 |
| | FWR2-a | LYWYRQDPGKSLESLFV | 977 |
| | FWR3-a | KQEGRLMASLDTKARLSTLHITAAVHDLSATYF | 978 |
| | FWR4-a | GKGTKLSVKP | 124 |
| TCRB- | b-Variable | AATGCTGGTGTCACTCAGACCCCAAAATTCCAGGTCCTGAAGACA | 1218 |
| 134 | nucleotide | GGACAGAGCATGACACTGCAGTGTGCCCAGGATATGAACCATAA | |
| | | CTCCATGTACTGGTATCGACAAGACCCAGGCATGGGACTGAGGCT | |
| | | GATTTATTACTCAGCTTCTGAGGGTACCACTGACAAAGGAGAAGT | |
| | | CCCCAATGGCTACAATGTCTCCAGATTAAACAAACGGGAGTTCTC | |
| | | GCTCAGGCTGGAGTCGGCTGCTCCCTCCCAGACATCTGTGTACTT | |
| | | CTGTGCCAGCAGTGACGGGGGTGAGCAGTTCTTCGGGCCAGGGA | |
| | | CACGGCTCACCGTGCTAG | |
| | Variable-b | NAGVTQTPKFQVLKTGQSMTLQCAQDMNHNSMYWYRQDPGMGL | 1219 |
| | | RLIYYSASEGTTDKGEVPNGYNVSRLNKREFSLRLESAAPSQTSVYF | |
| | | CASSDGGEQFFGPGTRLTVL | |
| | CDR1-b | MNHNS | 39 |
| | CDR2-b | SASEGT | 40 |
| | CDR3-b | CASSDGGEQFF | 1220 |
| | FWR1-b | NAGVTQTPKFQVLKTGQSMTLQCAQD | 42 |
| | FWR2-b | MYWYRQDPGMGLRLIYY | 43 |
| | FWR3-b | TDKGEVPNGYNVSRLNKREFSLRLESAAPSQTSVYF | 44 |
| | FWR4-b | GPGTRLTVL | 9 |
| TCRA- | a-Variable | GGACAAAACATTGACCAGCCCACTGAGATGACAGCTACGGAAGG | 1221 |
| 134 | nucleotide | TGCCATTGTCCAGATCAACTGCACGTACCAGACATCTGGGTTCAA | |
| | | CGGGCTGTTCTGGTACCAGCAACATGCTGGCGAAGCACCTACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTG | |
| | | AGAGATGGGGCTAGCAACTATCAGTTAATCTGGGGCGCTGGGAC | |
| | | CAAGCTAATTATAAAGCCAG | |
| | Variable-a | GQNIDQPTEMTATEGAIVQINCTYQTSGFNGLFWYQQHAGEAPTFLS | 1222 |
| | | YNVLDGLEEKGRFSSFLSRSKGYSYLLLKELQMKDSASYLCAVRDG | |
| | | ASNYQLIWGAGTKLIIKP | |
| | CDR1-a | TSGFNG | 450 |
| | CDR2-a | NVLDGL | 451 |
| | CDR3-a | CAVRDGASNYQLIW | 1223 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GAGTKLIIKP | 356 |
| TCRB- | b-Variable | GATGCCATGGTCATCCAGAACCCAAGATACCAGGTTACCCAGTTT | 1224 |
| 135 | nucleotide | GGAAAGCCAGTGACCCTGAGTTGTTCTCAGACTTTGAACCATAAC | |
| | | GTCATGTACTGGTACCAGCAGAAGTCAAGTCAGGCCCCAAAGCT | |
| | | GCTGTTCCACTACTATGACAAAGATTTTAACAATGAAGCAGACAC | |
| | | CCCTGATAACTTCCAATCCAGGAGGCCGAACACTTCTTTCTGCTTT | |
| | | CTTGACATCCGCTCACCAGGCCTGGGGGACGCAGCCATGTACCTG | |
| | | TGTGCCACCAGCAGTAACGGGTACAATGAGCAGTTCTTCGGGCCA | |
| | | GGGACACGGCTCACCGTGCTAG | |
| | Variable-b | DAMVIQNPRYQVTQFGKPVTLSCSQTLNHNVMYWYQQKSSQAPKL | 1225 |
| | | LFHYYDKDFNNEADTPDNFQSRRPNTSFCFLDIRSPGLGDAAMYLCA | |
| | | TSSNGYNEQFFGPGTRLTVL | |
| | CDR1-b | LNHNV | 3 |
| | CDR2-b | YYDKDF | 4 |
| | CDR3-b | CATSSNGYNEQFF | 1226 |
| | FWR1-b | DAMVIQNPRYQVTQFGKPVTLSCSQT | 6 |
| | FWR2-b | MYWYQQKSSQAPKLLFH | 7 |
| | FWR3-b | NNEADTPDNFQSRRPNTSFCFLDIRSPGLGDAAMYL | 8 |
| | FWR4-b | GPGTRLTVL | 9 |
| TCRA- | a-Variable | ACCCAGCTGCTGGAGCAGAGCCCTCAGTTTCTAAGCATCCAAGAG | 1227 |
| 135 | nucleotide | GGAGAAAATCTCACTGTGTACTGCAACTCCTCAAGTGTTTTTTCC | |
| | | AGCTTACAATGGTACAGACAGGAGCCTGGGGAAGGTCCTGTCCT | |
| | | CCTGGTGACAGTAGTTACGGGTGGAGAAGTGAAGAAGCTGAAGA | |
| | | GACTAACCTTTCAGTTTGGTGATGCAAGAAAGGACAGTTCTCTCC | |
| | | ACATCACTGCGGCCCAGCCTGGTGATACAGGCCTCTACCTCTGTG | |
| | | CAGGAGAGTTTTACTATTTTGGGACAGGGACAAGTTTGACGGTCA | |
| | | TTCCAA | |
| | Variable-a | TQLLEQSPQFLSIQEGENLTVYCNSSSVFSSLQWYRQEPGEGPVLLVT | 1228 |
| | | VVTGGEVKKLKRLTFQFGDARKDSSLHITAAQPGDTGLYLCAGEFY | |
| | | YFGTGTSLTVIP | |
| | CDR1-a | SVFSS | 533 |
| | CDR2-a | VVTGGEV | 534 |
| | CDR3-a | CAGEFYYF | 1229 |
| | FWR1-a | TQLLEQSPQFLSIQEGENLTVYCNSS | 536 |
| | FWR2-a | LQWYRQEPGEGPVLLVT | 537 |
| | FWR3-a | KKLKRLTFQFGDARKDSSLHITAAQPGDTGLYL | 538 |
| | FWR4-a | GTGTSLTVIP | 94 |
| TCRB- | b-Variable | AAGGCTGGAGTCACTCAAACTCCAAGATATCTGATCAAAACGAG | 1230 |
| 136 | nucleotide | AGGACAGCAAGTGACACTGAGCTGCTCCCCTATCTCTGGGCATAG | |
| | | GAGTGTATCCTGGTACCAACAGACCCCAGGACAGGGCCTTCAGTT | |
| | | CCTCTTTGAATACTTCAGTGAGACACAGAGAAACAAAGGAAACT | |
| | | TCCCTGGTCGATTCTCAGGGCGCCAGTTCTCTAACTCTCGCTCTGA | |
| | | GATGAATGTGAGCACCTTGGAGCTGGGGGACTCGGCCCTTTATCT | |
| | | TTGCGCCAGCAGCCGGGAGGGCGATCAGCCCCAGCATTTTGGTG | |
| | | ATGGGACTCGACTCTCCATCCTAG | |
| | Variable-b | KAGVTQTPRYLIKTRGQQVTLSCSPISGHRSVSWYQQTPGQGLQFLF | 1231 |
| | | EYFSETQRNKGNFPGRFSGRQFSNSRSEMNVSTLELGDSALYLCASS | |
| | | REGDQPQHFGDGTRLSIL | |
| | CDR1-b | SGHRS | 514 |
| | CDR2-b | YFSETQ | 515 |
| | CDR3-b | CASSREGDQPQHF | 1232 |
| | FWR1-b | KAGVTQTPRYLIKTRGQQVTLSCSPI | 517 |
| | FWR2-b | VSWYQQTPGQGLQFLFE | 518 |
| | FWR3-b | RNKGNFPGRFSGRQFSNSRSEMNVSTLELGDSALYL | 519 |
| | FWR4-b | GDGTRLSIL | 27 |
| TCRA- | a-Variable | GGAATACAAGTGGAGCAGAGTCCTCCAGACCTGATTCTCCAGGA | 1233 |
| 136 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTCCGC | |
| | | TATGGTCAGAATTTTGTCTTTGGTCCCGGAACCAGATTGTCCGTG | |
| | | CTGCCCT | |
| | Variable-a | GIQVEQSPPDLILQEGANSTLRCNFSDSVNNLQWFHQNPWGQLINLF | 1234 |
| | | YIPSGTKQNGRLSATTVATERYSLLYISSSQTTDSGVYFCAVRYGQN | |
| | | FVFGPGTRLSVLP | |
| | CDR1-a | DSVNN | 506 |
| | CDR2-a | IPSGT | 507 |
| | CDR3-a | CAVRYGQNFVF | 1235 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GPGTRLSVLP | 272 |
| TCRB- | b-Variable | AGTGCTGTCATCTCTCAAAAGCCAAGCAGGGATATCTGTCAACGT | 1236 |
| 137 | nucleotide | GGAACCTCCCTGACGATCCAGTGTCAAGTCGATAGCCAAGTCACC | |
| | | ATGATGTTCTGGTACCGTCAGCAACCTGGACAGAGCCTGACACTG | |
| | | ATCGCAACTGCAAATCAGGGCTCTGAGGCCACATATGAGAGTGG | |
| | | ATTTGTCATTGACAAGTTTCCCATCAGCCGCCCAAACCTAACATT | |
| | | CTCAACTCTGACTGTGAGCAACATGAGCCCTGAAGACAGCAGCA | |
| | | TATATCTCTGCAGCGTAGTCGGGACAGCCTCCTCTGGAAACACCA | |
| | | TATATTTTGGAGAGGGAAGTTGGCTCACTGTTGTAG | |
| | Variable-b | SAVISQKPSRDICQRGTSLTIQCQVDSQVTMMFWYRQQPGQSLTLIA | 1237 |
| | | TANQGSEATYESGFVIDKFPISRPNLTFSTLTVSNMSPEDSSIYLCSVV | |
| | | GTASSGNTIYFGEGSWLTVV | |
| | CDR1-b | SQVTM | 97 |
| | CDR2-b | ANQGSEA | 98 |
| | CDR3-b | CSVVGTASSGNTIYF | 1238 |
| | FWR1-b | SAVISQKPSRDICQRGTSLTIQCQVD | 100 |
| | FWR2-b | MFWYRQQPGQSLTLIAT | 101 |
| | FWR3-b | TYESGFVIDKFPISRPNLTFSTLTVSNMSPEDSSIYL | 102 |
| | FWR4-b | GEGSWLTVV | 871 |
| TCRA- | a-Variable | GCCCAGTCTGTGAGCCAGCATAACCACCATGTAATTCTCTCTGAA | 1239 |
| 137 | nucleotide | GCAGCCTCACTGGAGTTGGGATGCAACTATTCCTATGGTGGAACT | |
| | | GTTAATCTCTTCTGGTATGTCCAGTACCCTGGTCAACACCTTCAGC | |
| | | TTCTCCTCAAGTACTTTTCAGGGGATCCACTGGTTAAAGGCATCA | |
| | | AGGGCTTTGAGGCTGAATTTATAAAGAGTAAATTCTCCTTTAATC | |
| | | TGAGGAAACCCTCTGTGCAGTGGAGTGACACAGCTGAGTACTTCT | |
| | | GTGCCGTGAGGGGAACCGGTAACCAGTTCTATTTTGGGACAGGG | |
| | | ACAAGTTTGACGGTCATTCCAA | |
| | Variable-a | AQSVSQHNHHVILSEAASLELGCNYSYGGTVNLFWYVQYPGQHLQL | 1240 |
| | | LLKYFSGDPLVKGIKGFEAEFIKSKFSFNLRKPSVQWSDTAEYFCAV | |
| | | RGTGNQFYFGTGTSLTVIP | |
| | CDR1-a | YGGTVN | 1072 |
| | CDR2-a | YFSGDPLV | 1073 |
| | CDR3-a | CAVRGTGNQFYF | 1241 |
| | FWR1-a | AQSVSQHNHHVILSEAASLELGCNYS | 1075 |
| | FWR2-a | LFWYVQYPGQHLQLLLK | 1076 |
| | FWR3-a | KGIKGFEAEFIKSKFSFNLRKPSVQWSDTAEYF | 1077 |
| | FWR4-a | GTGTSLTVIP | 94 |
| TCRB- | b-Variable | AATGCTGGTGTCACTCAGACCCCAAAATTCCAGGTCCTGAAGACA | 1242 |
| 138 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGCCCCGGACAGGGAAGCTATGGCTACACCTTCG | |
| | | GTTCGGGGACCAGGTTAACCGTTGTAG | |
| | Variable-b | NAGVTQTPKFQVLKTGQSMTLQCAQDMNHEYMSWYRQDPGMGLR | 1243 |
| | | LIHYSVGAGITDQGEVPNGYNVSRSTTEDFPLRLLSAAPSQTSVYFCA | |
| | | SSPGQGSYGYTFGSGTRLTVV | |
| | CDR1-b | MNHEY | 252 |
| | CDR2-b | SVGAGI | 253 |
| | CDR3-b | CASSPGQGSYGYTF | 1244 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GSGTRLTVV | 103 |
| TCRA- | a-Variable | GCCCAGTCTGTGACCCAGCTTGACAGCCAAGTCCCTGTCTTTGAA | 1245 |
| 138 | nucleotide | GAAGCCCCTGTGGAGCTGAGGTGCAACTACTCATCGTCTGTTTCA | |
| | | GTGTATCTCTTCTGGTATGTGCAATACCCCAACCAAGGACTCCAG | |
| | | CTTCTCCTGAAGTATTTATCAGGATCCACCCTGGTTGAAAGCATC | |
| | | AACGGTTTTGAGGCTGAATTTAACAAGAGTCAAACTTCCTTCCAC | |
| | | TTGAGGAAACCCTCAGTCCATATAAGCGACACGGCTGAGTACTTC | |
| | | TGTGCTGTGATTCCGGATAGCAACTATCAGTTAATCTGGGGCGCT | |
| | | GGGACCAAGCTAATTATAAAGCCAG | |
| | Variable-a | AQSVTQLDSQVPVFEEAPVELRCNYSSSVSVYLFWYVQYPNQGLQL | 1246 |
| | | LLKYLSGSTLVESINGFEAEFNKSQTSFHLRKPSVHISDTAEYFCA VIP | |
| | | DSNYQLIWGAGTKLIIKP | |
| | CDR1-a | SSVSVY | 495 |
| | CDR2-a | YLSGSTLV | 496 |
| | CDR3-a | CAVIPDSNYQLIW | 1247 |
| | FWR1-a | AQSVTQLDSQVPVFEEAPVELRCNYS | 498 |
| | FWR2-a | LFWYVQYPNQGLQLLLK | 499 |
| | FWR3-a | ESINGFEAEFNKSQTSFHLRKPSVHISDTAEYF | 1248 |
| | FWR4-a | GAGTKLIIKP | 356 |
| TCRB- | b-Variable | GATGGTGGAATCACTCAGTCCCCAAAGTACCTGTTCAGAAAGGA | 1249 |
| 139 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGCCAAACCCGGACAGGCAATCAGCCCCAGC | |
| | | ATTTTGGTGATGGGACTCGACTCTCCATCCTAG | |
| | Variable-b | DGGITQSPKYLFRKEGQNVTLSCEQNLNHDAMYWYRQDPGQGLRLI | 1250 |
| | | YYSQIVNDFQKGDIAEGYSVSREKKESFPLTVTSAQKNPTAFYLCAS | |
| | | SQTRTGNQPQHFGDGTRLSIL | |
| | CDR1-b | LNHDA | 216 |
| | CDR2-b | SQIVND | 217 |
| | CDR3-b | CASSQTRTGNQPQHF | 1251 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GDGTRLSIL | 27 |
| TCRA- | a-Variable | GGAGAGAATGTGGAGCAGCATCCTTCAACCCTGAGTGTCCAGGA | 1252 |
| 139 | nucleotide | GGGAGACAGCGCTGTTATCAAGTGTACTTATTCAGACAGTGCCTC | |
| | | AAACTACTTCCCTTGGTATAAGCAAGAACTTGGAAAAGGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCAAGTACTGGTGCTACAAACAAGCTCATCTTTGGAACTG | |
| | | GCACTCTGCTTGCTGTCCAGCCAA | |
| | Variable-a | GENVEQHPSTLSVQEGDSAVIKCTYSDSASNYFPWYKQELGKGPQLI | 1253 |
| | | IDIRSNVGEKKDQRIA VTLNKTAKHFSLHITETQPEDSAVYFCAASTG | |
| | | ATNKLIFGTGTLLAVQP | |
| | CDR1-a | DSASNY | 207 |
| | CDR2-a | IRSNVGE | 208 |
| | CDR3-a | CAASTGATNKLIF | 1254 |
| | FWR1-a | GENVEQHPSTLSVQEGDSAVIKCTYS | 210 |
| | FWR2-a | FPWYKQELGKGPQLIID | 374 |
| | FWR3-a | KKDQRIAVTLNKTAKHFSLHITETQPEDSAVYF | 212 |
| | FWR4-a | GTGTLLAVQP | 480 |
| TCRB- | b-Variable | GAAGCTGGAGTTGCCCAGTCTCCCAGATATAAGATTATAGAGAA | 1255 |
| 140 | nucleotide | AAGGCAGAGTGTGGCTTTTTGGTGCAATCCTATATCTGGCCATGC | |
| | | TACCCTTTACTGGTACCAGCAGATCCTGGGACAGGGCCCAAAGCT | |
| | | TCTGATTCAGTTTCAGAATAACGGTGTAGTGGATGATTCACAGTT | |
| | | GCCTAAGGATCGATTTTCTGCAGAGAGGCTCAAAGGAGTAGACT | |
| | | CCACTCTCAAGATCCAGCCTGCAAAGCTTGAGGACTCGGCCGTGT | |
| | | ATCTCTGTGCCAGCAGCTTAGTTGGACAGGGGGCCTACAATGAGC | |
| | | AGTTCTTCGGGCCAGGGACACGGCTCACCGTGCTAG | |
| | Variable-b | EAGVAQSPRYKIIEKRQSVAFWCNPISGHATLYWYQQILGQGPKLLI | 1256 |
| | | QFQNNGVVDDSQLPKDRFSAERLKGVDSTLKIQPAKLEDSAVYLCA | |
| | | SSLVGQGAYNEQFFGPGTRLTVL | |
| | CDR1-b | SGHAT | 894 |
| | CDR2-b | FQNNGV | 895 |
| | CDR3-b | CASSLVGQGAYNEQFF | 1257 |
| | FWR1-b | EAGVAQSPRYKIIEKRQSVAFWCNPI | 897 |
| | FWR2-b | LYWYQQILGQGPKLLIQ | 898 |
| | FWR3-b | VDDSQLPKDRFSAERLKGVDSTLKIQPAKLEDSAVYL | 899 |
| | FWR4-b | GPGTRLTVL | 9 |
| TCRA- | a-Variable | CGGAAGGAGGTGGAGCAGGATCCTGGACCCTTCAATGTTCCAGA | 1258 |
| 140 | nucleotide | GGGAGCCACTGTCGCTTTCAACTGTACTTACAGCAACAGTGCTTC | |
| | | TCAGTCTTTCTTCTGGTACAGACAGGATTGCAGGAAAGAACCTAA | |
| | | GTTGCTGATGTCCGTATACTCCAGTGGTAATGAAGATGGAAGGTT | |
| | | TACAGCACAGCTCAATAGAGCCAGCCAGTATATTTCCCTGCTCAT | |
| | | CAGAGACTCCAAGCTCAGTGATTCAGCCACCTACCTCTGTGTGGT | |
| | | GAACCCTCATGGCTCTAGCAACACAGGCAAACTAATCTTTGGGCA | |
| | | AGGGACAACTTTACAAGTAAAACCAG | |
| | Variable-a | RKEVEQDPGPFNVPEGATVAFNCTYSNSASQSFFWYRQDCRKEPKL | 1259 |
| | | LMSVYSSGNEDGRFTAQLNRASQYISLLIRDSKLSDSATYLCVVNPH | |
| | | GSSNTGKLIFGQGTTLQVKP | |
| | CDR1-a | NSASQS | 224 |
| | CDR2-a | VYSSG | 225 |
| | CDR3-a | CVVNPHGSSNTGKLIF | 1260 |
| | FWR1-a | RKEVEQDPGPFNVPEGATVAFNCTYS | 227 |
| | FWR2-a | FFWYRQDCRKEPKLLMS | 228 |
| | FWR3-a | NEDGRFTAQLNRASQYISLLIRDSKLSDSATYL | 229 |
| | FWR4-a | GQGTTLQVKP | 1261 |
| TCRB- | b-Variable | AATGCTGGTGTCACTCAGACCCCAAAATTCCGGGTCCTGAAGACA | 1262 |
| 141 | nucleotide | GGACAGAGCATGACACTGCTGTGTGCCCAGGATATGAACCATGA | |
| | | ATACATGTACTGGTATCGACAAGACCCAGGCATGGGGCTGAGGC | |
| | | TGATTCATTACTCAGTTGGTGAGGGTACAACTGCCAAAGGAGAG | |
| | | GTCCCTGATGGCTACAATGTCTCCAGATTAAAAAAACAGAATTTC | |
| | | CTGCTGGGGTTGGAGTCGGCTGCTCCCTCCCAAACATCTGTGTAC | |
| | | TTCTGTGCCAGCAGCTTCATCGGTTGGGGCTTAGCACCCCGGGGG | |
| | | GCCCAGTACTTCGGGCCAGGCACGCGGCTCCTGGTGCTCG | |
| | Variable-b | NAGVTQTPKFRVLKTGQSMTLLCAQDMNHEYMYWYRQDPGMGLR | 1263 |
| | | LIHYSVGEGTTAKGEVPDGYNVSRLKKQNFLLGLESAAPSQTSVYFC | |
| | | ASSFIGWGLAPRGAQYFGPGTRLLVL | |
| | CDR1-b | MNHEY | 252 |
| | CDR2-b | SVGEGT | 1046 |
| | CDR3-b | CASSFIGWGLAPRGAQYF | 1264 |
| | FWR1-b | NAGVTQTPKFRVLKTGQSMTLLCAQD | 1048 |
| | FWR2-b | MYWYRQDPGMGLRLIHY | 1049 |
| | FWR3-b | TAKGEVPDGYNVSRLKKQNFLLGLESAAPSQTSVYF | 1050 |
| | FWR4-b | GPGTRLLVL | 67 |
| TCRA- | a-Variable | GCTCAGTCAGTGGCTCAGCCGGAAGATCAGGTCAACGTTGCTGA | 1265 |
| 141 | nucleotide | AGGGAATCCTCTGACTGTGAAATGCACCTATTCAGTCTCTGGAAA | |
| | | CCCTTATCTTTTTTGGTATGTTCAATACCCCAACCGAGGCCTCCAG | |
| | | TTCCTTCTGAAATACATCACAGGGGATAACCTGGTTAAAGGCAGC | |
| | | TATGGCTTTGAAGCTGAATTTAACAAGAGCCAAACCTCCTTCCAC | |
| | | CTGAAGAAACCATCTGCCCTTGTGAGCGACTCCGCTTTGTACTTC | |
| | | TGTGCTGTGAGATTCGACAGAGGCTCAACCCTGGGGAGGCTATAC | |
| | | TTTGGAAGAGGAACTCAGTTGACTGTCTGGCCTG | |
| | Variable-a | AQSVAQPEDQVNVAEGNPLTVKCTYSVSGNPYLFWYVQYPNRGLQ | 1266 |
| | | FLLKYITGDNLVKGSYGFEAEFNKSQTSFHLKKPSALVSDSALYFCA | |
| | | VRFDRGSTLGRLYFGRGTQLTVWP | |
| | CDR1-a | VSGNPY | 427 |
| | CDR2-a | YITGDNLV | 428 |
| | CDR3-a | CAVRFDRGSTLGRLYF | 1267 |
| | FWR1-a | AQSVAQPEDQVNVAEGNPLTVKCTYS | 430 |
| | FWR2-a | LFWYVQYPNRGLQFLLK | 431 |
| | FWR3-a | KGSYGFEAEFNKSQTSFHLKKPSALVSDSALYF | 432 |
| | FWR4-a | GRGTQLTVWP | 190 |
| TCRB- | b-Variable | GACACTGAAGTTACCCAGACACCAAAACACCTGGTCATGGGAAT | 1268 |
| 142 | nucleotide | GACAAATAAGAAGTCTTTGAAATGTGAACAACATATGGGGCACA | |
| | | GGGCTATGTATTGGTACAAGCAGAAAGCTAAGAAGCCACCGGAG | |
| | | CTCATGTTTGTCTACAGCTATGAGAAACTCTCTATAAATGAAAGT | |
| | | GTGCCAAGTCGCTTCTCACCTGAATGCCCCAACAGCTCTCTCTTA | |
| | | AACCTTCACCTACACGCCCTGCAGCCAGAAGACTCAGCCCTGTAT | |
| | | CTCTGCGCCAGCAGCCTGAGAGAAAAACTGTTTTTTGGCAGTGGA | |
| | | ACCCAGCTCTCTGTCTTGG | |
| | Variable-b | DTEVTQTPKHLVMGMTNKKSLKCEQHMGHRAMYWYKQKAKKPP | 1269 |
| | | ELMFVYSYEKLSINESVPSRFSPECPNSSLLNLHLHALQPEDSALYLC | |
| | | ASSLREKLFFGSGTQLSVL | |
| | CDR1-b | MGHRA | 639 |
| | CDR2-b | YSYEKL | 640 |
| | CDR3-b | CASSLREKLFF | 1270 |
| | FWR1-b | DTEVTQTPKHLVMGMTNKKSLKCEQH | 642 |
| | FWR2-b | MYWYKQKAKKPPELMFV | 643 |
| | FWR3-b | SINESVPSRFSPECPNSSLLNLHLHALQPEDSALYL | 644 |
| | FWR4-b | GSGTQLSVL | 45 |
| TCRA- | a-Variable | GGAGAGGATGTGGAGCAGAGTCTTTTCCTGAGTGTCCGAGAGGG | 1271 |
| 142 | nucleotide | AGACAGCTCCGTTATAAACTGCACTTACACAGACAGCTCCTCCAC | |
| | | CTACTTATACTGGTATAAGCAAGAACCTGGAGCAGGTCTCCAGTT | |
| | | GCTGACGTATATTTTTTCAAATATGGACATGAAACAAGACCAAAG | |
| | | ACTCACTGTTCTATTGAATAAAAAGGATAAACATCTGTCTCTGCG | |
| | | CATTGCAGACACCCAGACTGGGGACTCAGCTATCTACTTCTGTGC | |
| | | AGAAAATGCTGGCAACAACCGTAAGCTGATTTGGGGATTGGGAA | |
| | | CAAGCCTGGCAGTAAATCCGA | |
| | Variable-a | GEDVEQSLFLSVREGDSSVINCTYTDSSSTYLYWYKQEPGAGLQLLT | 1272 |
| | | YIFSNMDMKQDQRLTVLLNKKDKHLSLRIADTQTGDSAIYFCAENA | |
| | | GNNRKLIWGLGTSLAVNP | |
| | CDR1-a | DSSSTY | 314 |
| | CDR2-a | IFSNMDM | 315 |
| | CDR3-a | CAENAGNNRKLIW | 1273 |
| | FWR1-a | GEDVEQSLFLSVREGDSSVINCTYT | 317 |
| | FWR2-a | LYWYKQEPGAGLQLLTY | 318 |
| | FWR3-a | KQDQRLTVLLNKKDKHLSLRIADTQTGDSAIYF | 319 |
| | FWR4-a | GLGTSLAVNP | 242 |
| TCRB- | b-Variable | GATGCTGGAGTTATCCAGTCACCCCGGCACGAGGTGACAGAGAT | 1274 |
| 143 | nucleotide | GGGACAAGAAGTGACTCTGAGATGTAAACCAATTTCAGGACACG | |
| | | ACTACCTTTTCTGGTACAGACAGACCATGATGCGGGGACTGGAGT | |
| | | TGCTCATTTACTTTAACAACAACGTTCCGATAGATGATTCAGGGA | |
| | | TGCCCGAGGATCGATTCTCAGCTAAGATGCCTAATGCATCATTCT | |
| | | CCACTCTGAAGATCCAGCCCTCAGAACCCAGGGACTCAGCTGTGT | |
| | | ACTTCTGTGCCAGCAGTCGGGGACAGGGTGTCTCCTACGAGCAGT | |
| | | ACTTCGGGCCGGGCACCAGGCTCACGGTCACAG | |
| | Variable-b | DAGVIQSPRHEVTEMGQEVTLRCKPISGHDYLFWYRQTMMRGLELL | 1275 |
| | | IYFNNNVPIDDSGMPEDRFSAKMPNASFSTLKIQPSEPRDSAVYFCAS | |
| | | SRGQGVSYEQYFGPGTRLTVT | |
| | CDR1-b | SGHDY | 468 |
| | CDR2-b | FNNNVP | 469 |
| | CDR3-b | CASSRGQGVSYEQYF | 1276 |
| | FWR1-b | DAGVIQSPRHEVTEMGQEVTLRCKPI | 471 |
| | FWR2-b | LFWYRQTMMRGLELLIY | 472 |
| | FWR3-b | IDDSGMPEDRFSAKMPNASFSTLKIQPSEPRDSAVYF | 473 |
| | FWR4-b | GPGTRLTVT | 85 |
| TCRA- | a-Variable | CGGAAGGAGGTGGAGCAGGATCCTGGACCCTTCAATGTTCCAGA | 1277 |
| 143 | nucleotide | GGGAGCCACTGTCGCTTTCAACTGTACTTACAGCAACAGTGCTTC | |
| | | TCAGTCTTTCTTCTGGTACAGACAGGATTGCAGGAAAGAACCTAA | |
| | | GTTGCTGATGTCCGTATACTCCAGTGGTAATGAAGATGGAAGGTT | |
| | | TACAGCACAGCTCAATAGAGCCAGCCAGTATATTTCCCTGCTCAT | |
| | | CAGAGACTCCAAGCTCAGTGATTCAGCCACCTACCTCTGTGTGGT | |
| | | GAGGGCTAACTTTGGAAATGAGAAATTAACCTTTGGGACTGGAA | |
| | | CAAGACTCACCATCATACCCA | |
| | Variable-a | RKEVEQDPGPFNVPEGATVAFNCTYSNSASQSFFWYRQDCRKEPKL | 1278 |
| | | LMSVYSSGNEDGRFTAQLNRASQYISLLIRDSKLSDSATYLCVVRAN | |
| | | FGNEKLTFGTGTRLTIIP | |
| | CDR1-a | NSASQS | 224 |
| | CDR2-a | VYSSG | 225 |
| | CDR3-a | CVVRANFGNEKLTF | 1279 |
| | FWR1-a | RKEVEQDPGPFNVPEGATVAFNCTYS | 227 |
| | FWR2-a | FFWYRQDCRKEPKLLMS | 228 |
| | FWR3-a | NEDGRFTAQLNRASQYISLLIRDSKLSDSATYL | 229 |
| | FWR4-a | GTGTRLTIIP | 292 |
| TCRB- | b-Variable | GGTGCTGTCGTCTCTCAACATCCGAGCTGGGTTATCTGTAAGAGT | 1280 |
| 144 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGTGGACAGACAGATACGCAGTATT | |
| | | TTGGCCCAGGCACCCGGCTGACAGTGCTCG | |
| | Variable-b | GAVVSQHPSWVICKSGTSVKIECRSLDFQATTMFWYRQFPKQSLML | 1281 |
| | | MATSNEGSKATYEQGVEKDKFLINHASLTLSTLTVTSAHPEDSSFYIC | |
| | | SASGQTDTQYFGPGTRLTVL | |
| | CDR1-b | DFQATT | 79 |
| | CDR2-b | SNEGSKA | 80 |
| | CDR3-b | CSASGQTDTQYF | 1282 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVL | 9 |
| TCRA- | a-Variable | CAGAAGGAGGTGGAGCAGGATCCTGGACCACTCAGTGTTCCAGA | 1283 |
| 144 | nucleotide | GGGAGCCATTGTTTCTCTCAACTGCACTTACAGCAACAGTGCTTT | |
| | | TCAATACTTCATGTGGTACAGACAGTATTCCAGAAAAGGCCCTGA | |
| | | GTTGCTGATGTACACATACTCCAGTGGTAACAAAGAAGATGGAA | |
| | | GGTTTACAGCACAGGTCGATAAATCCAGCAAGTATATCTCCTTGT | |
| | | TCATCAGAGACTCACAGCCCAGTGATTCAGCCACCTACCTCTGTG | |
| | | CAATGAGGGCTTATACTGGAGGCTTCAAAACTATCTTTGGAGCAG | |
| | | GAACAAGACTATTTGTTAAAGCAA | |
| | Variable-a | QKEVEQDPGPLSVPEGAIVSLNCTYSNSAFQYFMWYRQYSRKGPEL | 1284 |
| | | LMYTYSSGNKEDGRFTAQVDKSSKYISLFIRDSQPSDSATYLCAMRA | |
| | | YTGGFKTIFGAGTRLFVKA | |
| | CDR1-a | NSAFQY | 673 |
| | CDR2-a | TYSSGN | 674 |
| | CDR3-a | CAMRAYTGGFKTIF | 1285 |
| | FWR1-a | QKEVEQDPGPLSVPEGAIVSLNCTYS | 676 |
| | FWR2-a | FMWYRQYSRKGPELLMY | 677 |
| | FWR3-a | KEDGRFTAQVDKSSKYISLFIRDSQPSDSATYL | 678 |
| | FWR4-a | GAGTRLFVKA | 112 |
| TCRB- | b-Variable | GGTGCTGTCGTCTCTCAACATCCGAGCTGGGTTATCTGTAAGAGT | 1286 |
| 145 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGGCCGAGGGGCAACACTGAAGCTT | |
| | | TCTTTGGACAAGGCACCAGACTCACAGTTGTAG | |
| | Variable-b | GAVVSQHPSWVICKSGTSVKIECRSLDFQATTMFWYRQFPKQSLML | 1287 |
| | | MATSNEGSKATYEQGVEKDKFLINHASLTLSTLTVTSAHPEDSSFYIC | |
| | | SARPRGNTEAFFGQGTRLTVV | |
| | CDR1-b | DFQATT | 79 |
| | CDR2-b | SNEGSKA | 80 |
| | CDR3-b | CSARPRGNTEAFF | 1288 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GQGTRLTVV | 246 |
| TCRA- | a-Variable | GGAATACAAGTGGAGCAGAGTCCTCCAGACCTGATTCTCCAGGA | 1289 |
| 145 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTGCG | |
| | | GCTGAATTATGGTGGTGCTACAAACAAGCTCATCTTTGGAACTGG | |
| | | CACTCTGCTTGCTGTCCAGCCAA | |
| | Variable-a | GIQVEQSPPDLILQEGANSTLRCNFSDSVNNLQWFHQNPWGQLINLF | 1290 |
| | | YIPSGTKQNGRLSATTVATERYSLLYISSSQTTDSGVYFCAVRLNYG | |
| | | GATNKLIFGTGTLLAVQP | |
| | CDR1-a | DSVNN | 506 |
| | CDR2-a | IPSGT | 507 |
| | CDR3-a | CAVRLNYGGATNKLIF | 1291 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GTGTLLAVQP | 480 |
| TCRB- | b-Variable | GGTGCTGTCGTCTCTCAACATCCGAGCTGGGTTATCTGTAAGAGT | 1292 |
| 146 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGACCGCCTCAGGGGGACACTGAAG | |
| | | CTTTCTTTGGACAAGGCACCAGACTCACAGTTGTAG | |
| | Variable-b | GAVVSQHPSWVICKSGTSVKIECRSLDFQATTMFWYRQFPKQSLML | 1293 |
| | | MATSNEGSKATYEQGVEKDKFLINHASLTLSTLTVTSAHPEDSSFYIC | |
| | | SARPPQGDTEAFFGQGTRLTVV | |
| | CDR1-b | DFQATT | 79 |
| | CDR2-b | SNEGSKA | 80 |
| | CDR3-b | CSARPPQGDTEAFF | 1294 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GQGTRLTVV | 246 |
| TCRA- | a-Variable | GGACAAAACATTGACCAGCCCACTGAGATGACAGCTACGGAAGG | 1295 |
| 146 | nucleotide | TGCCATTGTCCAGATCAACTGCACGTACCAGACATCTGGGTTCAA | |
| | | CGGGCTGTTCTGGTACCAGCAACATGCTGGCGAAGCACCCACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTT | |
| | | ATGGATAGCAACTATCAGTTAATCTGGGGCGCTGGGACCAAGCT | |
| | | AATTATAAAGCCAG | |
| | Variable-a | GQNIDQPTEMTATEGAIVQINCTYQTSGFNGLFWYQQHAGEAPTFLS | 1296 |
| | | YNVLDGLEEKGRESSFLSRSKGYSYLLLKELQMKDSASYLCAVMDS | |
| | | NYQLIWGAGTKLIIKP | |
| | CDR1-a | TSGFNG | 450 |
| | CDR2-a | NVLDGL | 451 |
| | CDR3-a | CAVMDSNYQLIW | 1297 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GAGTKLIIKP | 356 |
| TCRB- | b-Variable | GATGCTGATGTTACCCAGACCCCAAGGAATAGGATCACAAAGAC | 1298 |
| 147 | nucleotide | AGGAAAGAGGATTATGCTGGAATGTTCTCAGACTAAGGGTCATG | |
| | | ATAGAATGTACTGGTATCGACAAGACCCAGGACTGGGCCTACGG | |
| | | TTGATCTATTACTCCTTTGATGTCAAAGATATAAACAAAGGAGAG | |
| | | ATCTCTGATGGATACAGTGTCTCTCGACAGGCACAGGCTAAATTC | |
| | | TCCCTGTCCCTAGAGTCTGCCATCCCCAACCAGACAGCTCTTTACT | |
| | | TCTGTGCCACTCAGGGTAGCAATCAGCCCCAGCATTTTGGTGATG | |
| | | GGACTCGACTCTCCATCCTAG | |
| | Variable-b | DADVTQTPRNRITKTGKRIMLECSQTKGHDRMYWYRQDPGLGLRLI | 1299 |
| | | YYSFDVKDINKGEISDGYSVSRQAQAKFSLSLESAIPNQTALYFCATQ | |
| | | GSNQPQHFGDGTRLSIL | |
| | CDR1-b | KGHDR | 21 |
| | CDR2-b | SFDVKD | 22 |
| | CDR3-b | CATQGSNQPQHF | 1300 |
| | FWR1-b | DADVTQTPRNRITKTGKRIMLECSQT | 24 |
| | FWR2-b | MYWYRQDPGLGLRLIYY | 25 |
| | FWR3-b | INKGEISDGYSVSRQAQAKFSLSLESAIPNQTALYF | 26 |
| | FWR4-b | GDGTRLSIL | 27 |
| TCRA- | a-Variable | CAGAAGGAGGTGGAGCAGAATTCTGGACCCCTCAGTGTTCCAGA | 1301 |
| 147 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTCCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAACGCTGCAGGCAACAAGCTAACTTTTGGAGGAGGAACC | |
| | | AGGGTGCTAGTTAAACCAA | |
| | Variable-a | QKEVEQNSGPLSVPEGAIASLNCTYSDRGSQSFFWYRQYSGKSPELI | 1302 |
| | | MSIYSNGDKEDGRFTAQLNKASQYVSLLIRDSQPSDSATYLCAVNA | |
| | | AGNKLTFGGGTRVLVKP | |
| | CDR1-a | DRGSQS | 12 |
| | CDR2-a | IYSNGD | 13 |
| | CDR3-a | CAVNAAGNKLTF | 1303 |
| | FWR1-a | QKEVEQNSGPLSVPEGAIASLNCTYS | 15 |
| | FWR2-a | FFWYRQYSGKSPELIMS | 1304 |
| | FWR3-a | KEDGRFTAQLNKASQYVSLLIRDSQPSDSATYL | 17 |
| | FWR4-a | GGGTRVLVKP | 1103 |
| TCRB- | b-Variable | GATGGTGGAATCACTCAGTCCCCAAAGTACCTGTTCAGAAAGGA | 1305 |
| 148 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCACTTCGGTTTATTACAATGAGCAGTTCTTCGGGCCA | |
| | | GGGACACGGCTCACCGTGCTAG | |
| | Variable-b | DGGITQSPKYLFRKEGQNVTLSCEQNLNHDAMYWYRQDPGQGLRLI | 1306 |
| | | YYSQIVNDFQKGDIAEGYSVSREKKESFPLTVTSAQKNPTAFYLCAT | |
| | | SVYYNEQFFGPGTRLTVL | |
| | CDR1-b | LNHDA | 216 |
| | CDR2-b | SQIVND | 217 |
| | CDR3-b | CATSVYYNEQFF | 1307 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVL | 9 |
| TCRA- | a-Variable | CAGAAGGAGGTGGAGCAGGATCCTGGACCACTCAGTGTTCCAGA | 1308 |
| 148 | nucleotide | GGGAGCCATTGTTTCTCTCAACTGCACTTACAGCAACAGTGCTTT | |
| | | TCAATACTTCATGTGGTACAGACAGTATTCCAGAAAAGGCCCTGA | |
| | | GTTGCTGATGTACACATACTCCAGTGGTAACAAAGAAGATGGAA | |
| | | GGTTTACAGCACAGGTCGATAAATCCAGCAAGTATATCTCCTTGT | |
| | | TCATCAGAGACTCACAGCCCAGTGATTCAGCCACCTACCTCTGTG | |
| | | CAATGACCGATAACACCGACAAGCTCATCTTTGGGACTGGGACC | |
| | | AGATTACAAGTCTTTCCAA | |
| | Variable-a | QKEVEQDPGPLSVPEGAIVSLNCTYSNSAFQYFMWYRQYSRKGPEL | 1309 |
| | | LMYTYSSGNKEDGRFTAQVDKSSKYISLFIRDSQPSDSATYLCAMTD | |
| | | NTDKLIFGTGTRLQVFP | |
| | CDR1-a | NSAFQY | 673 |
| | CDR2-a | TYSSGN | 674 |
| | CDR3-a | CAMTDNTDKLIF | 1310 |
| | FWR1-a | QKEVEQDPGPLSVPEGAIVSLNCTYS | 676 |
| | FWR2-a | FMWYRQYSRKGPELLMY | 677 |
| | FWR3-a | KEDGRFTAQVDKSSKYISLFIRDSQPSDSATYL | 678 |
| | FWR4-a | GTGTRLQVFP | 36 |
| TCRB- | b-Variable | GGTGCTGTCGTCTCTCAACATCCGAGCTGGGTTATCTGTAAGAGT | 1311 |
| 149 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTCGACTGTGGGGGCTAACCTACGAGC | |
| | | AGTACTTCGGGCCGGGCACCAGGCTCACGGTCACAG | |
| | Variable-b | GAVVSQHPSWVICKSGTSVKIECRSLDFQATTMFWYRQFPKQSLML | 1312 |
| | | MATSNEGSKATYEQGVEKDKFLINHASLTLSTLTVTSAHPEDSSFYIC | |
| | | SARLWGLTYEQYFGPGTRLTVT | |
| | CDR1-b | DFQATT | 79 |
| | CDR2-b | SNEGSKA | 80 |
| | CDR3-b | CSARLWGLTYEQYF | 1313 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVT | 85 |
| TCRA- | a-Variable | CAGAAGGAGGTGGAGCAGAATTCTGGACCCCTCAGTGTTCCAGA | 1314 |
| 149 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTCCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAACACACCATATGGAAACAAACTGGTCTTTGGCGCAGGA | |
| | | ACCATTCTGAGAGTCAAGTCCT | |
| | Variable-a | QKEVEQNSGPLSVPEGAIASLNCTYSDRGSQSFFWYRQYSGKSPELI | 1315 |
| | | MSIYSNGDKEDGRFTAQLNKASQYVSLLIRDSQPSDSATYLCAVNTP | |
| | | YGNKLVFGAGTILRVKS | |
| | CDR1-a | DRGSQS | 12 |
| | CDR2-a | IYSNGD | 13 |
| | CDR3-a | CAVNTPYGNKLVF | 1316 |
| | FWR1-a | QKEVEQNSGPLSVPEGAIASLNCTYS | 15 |
| | FWR2-a | FFWYRQYSGKSPELIMS | 1304 |
| | FWR3-a | KEDGRFTAQLNKASQYVSLLIRDSQPSDSATYL | 17 |
| | FWR4-a | GAGTILRVKS | 18 |
| TCRB- | b-Variable | GGTGCTGTCGTCTCTCAACATCCGAGCTGGGTTATCTGTAAGAGT | 1317 |
| 150 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGAGATCGGTGGGCAGGCTATGGCT | |
| | | ACACCTTCGGTTCGGGGACCAGGTTAACCGTTGTAG | |
| | Variable-b | GAVVSQHPSWVICKSGTSVKIECRSLDFQATTMFWYRQFPKQSLML | 1318 |
| | | MATSNEGSKATYEQGVEKDKFLINHASLTLSTLTVTSAHPEDSSFYIC | |
| | | SARDRWAGYGYTFGSGTRLTVV | |
| | CDR1-b | DFQATT | 79 |
| | CDR2-b | SNEGSKA | 80 |
| | CDR3-b | CSARDRWAGYGYTF | 1319 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GSGTRLTVV | 103 |
| TCRA- | a-Variable | GCTCAGACAGTCACTCAGTCTCAACCAGAGATGTCTGTGCAGGAG | 1320 |
| 150 | nucleotide | GCAGAGACCGTGACCCTGAGCTGCACATATGACACCAGTGAGAG | |
| | | TGATTATTATTTATTCTGGTACAAGCAGCCTCCCAGCAGGCAGAT | |
| | | GATTCTCGTTATTCGCCAAGAAGCTTATAAGCAACAGAATGCAAC | |
| | | AGAGAATCGTTTCTCTGTGAACTTCCAGAAAGCAGCCAAATCCTT | |
| | | CAGTCTCAAGATCTCAGACTCACAGCTGGGGGATGCCGCGATGTA | |
| | | TTTCTGTGCCCAATACAGTAACAATAACAATGACATGCGCTTTGG | |
| | | AGCAGGGACCAGACTGACAGTAAAACCAA | |
| | Variable-a | AQTVTQSQPEMSVQEAETVTLSCTYDTSESDYYLFWYKQPPSRQMI | 1321 |
| | | LVIRQEAYKQQNATENRFSVNFQKAAKSFSLKISDSQLGDAAMYFC | |
| | | AQYSNNNNDMRFGAGTRLTVKP | |
| | CDR1-a | TSESDYY | 286 |
| | CDR2-a | QEAYKQQN | 287 |
| | CDR3-a | CAQYSNNNNDMRF | 1322 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDAAMYF | 291 |
| | FWR4-a | GAGTRLTVKP | 685 |
| TCRB- | b-Variable | GATGGTGGAATCACTCAGTCCCCAAAGTACCTGTTCAGAAAGGA | 1323 |
| 151 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGCCTACAGGGGACTAGAGGCTACACCTTCGG | |
| | | TTCGGGGACCAGGTTAACCGTTGTAG | |
| | Variable-b | DGGITQSPKYLFRKEGQNVTLSCEQNLNHDAMYWYRQDPGQGLRLI | 1324 |
| | | YYSQIVNDFQKGDIAEGYSVSREKKESFPLTVTSAQKNPTAFYLCAS | |
| | | SLQGTRGYTFGSGTRLTVV | |
| | CDR1-b | LNHDA | 216 |
| | CDR2-b | SQIVND | 217 |
| | CDR3-b | CASSLQGTRGYTF | 1325 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GSGTRLTVV | 103 |
| TCRA- | a-Variable | GCCCAGAGAGTGACTCAGCCCGAGAAGCTCCTCTCTGTCTTTAAA | 1326 |
| 151 | nucleotide | GGGGCCCCAGTGGAGCTGAAGTGCAACTATTCCTATTCTGGGAGT | |
| | | CCTGAACTCTTCTGGTATGTCCAGTACTCCAGACAACGCCTCCAG | |
| | | TTACTCTTGAGACACATCTCTAGAGAGAGCATCAAAGGCTTCACT | |
| | | GCTGACCTTAACAAAGGCGAGACATCTTTCCACCTGAAGAAACC | |
| | | ATTTGCTCAAGAGGAAGACTCAGCCATGTATTACTGTGCTCTCCT | |
| | | ATATGGAAACAAGCTGGTCTTTGGCGCAGGAACCATTCTGAGAGT | |
| | | CAAGTCCT | |
| | Variable-a | AQRVTQPEKLLSVFKGAPVELKCNYSYSGSPELFWYVQYSRQRLQL | 1327 |
| | | LLRHISRESIKGFTADLNKGETSFHLKKPFAQEEDSAMYYCALLYGN | |
| | | KLVFGAGTILRVKS | |
| | CDR1-a | YSGSPE | 70 |
| | CDR2-a | HISR | 71 |
| | CDR3-a | CALLYGNKLVF | 1328 |
| | FWR1-a | AQRVTQPEKLLSVFKGAPVELKCNYS | 73 |
| | FWR2-a | LFWYVQYSRQRLQLLLR | 74 |
| | FWR3-a | ESIKGFTADLNKGETSFHLKKPFAQEEDSAMYY | 75 |
| | FWR4-a | GAGTILRVKS | 18 |
| TCRB- | b-Variable | GAAACGGGAGTTACGCAGACACCAAGACACCTGGTCATGGGAAT | 1329 |
| 152 | nucleotide | GACAAATAAGAAGTCTTTGAAATGTGAACAACATCTGGGGCATA | |
| | | ACGCTATGTATTGGTACAAGCAAAGTGCTAAGAAGCCACTGGAG | |
| | | CTCATGTTTGTCTACAACTTTAAAGAACAGACTGAAAACAACAGT | |
| | | GTGCCAAGTCGCTTCTCACCTGAATGCCCCAACAGCTCTCACTTA | |
| | | TTCCTTCACCTACACACCCTGCAGCCAGAAGACTCGGCCCTGTAT | |
| | | CTCTGTGCCAGCAGCCAAGTGTCCACCGGGGAGCTGTTTTTTGGA | |
| | | GAAGGCTCTAGGCTGACCGTACTGG | |
| | Variable-b | ETGVTQTPRHLVMGMTNKKSLKCEQHLGHNAMYWYKQSAKKPLE | 1330 |
| | | LMFVYNFKEQTENNSVPSRFSPECPNSSHLFLHLHTLQPEDSALYLC | |
| | | ASSQVSTGELFFGEGSRLTVL | |
| | CDR1-b | LGHNA | 169 |
| | CDR2-b | YNFKEQ | 170 |
| | CDR3-b | CASSQVSTGELFF | 1331 |
| | FWR1-b | ETGVTQTPRHLVMGMTNKKSLKCEQH | 172 |
| | FWR2-b | MYWYKQSAKKPLELMFV | 173 |
| | FWR3-b | TENNSVPSRFSPECPNSSHLFLHLHTLQPEDSALYL | 174 |
| | FWR4-b | GEGSRLTVL | 63 |
| TCRA- | a-Variable | AAGGACCAAGTGTTTCAGCCTTCCACAGTGGCATCTTCAGAGGGA | 1332 |
| 152 | nucleotide | GCTGTGGTGGAAATCTTCTGTAATCACTCTGTGTCCAATGCTTAC | |
| | | AACTTCTTCTGGTACCTTCACTTCCCGGGATGTGCACCAAGACTC | |
| | | CTTGTTAAAGGCTCAAAGCCTTCTCAGCAGGGACGATACAACATG | |
| | | ACCTATGAACGGTTCTCTTCATCGCTGCTCATCCTCCAGGTGCGG | |
| | | GAGGCAGATGCTGCTGTTTACTACTGTGCTGTTGTTTCTGGTGGCT | |
| | | ACAATAAGCTGATTTTTGGAGCAGGGACCAGGCTGGCTGTACACC | |
| | | CAT | |
| | Variable-a | KDQVFQPSTVASSEGAVVEIFCNHSVSNAYNFFWYLHFPGCAPRLLV | 1333 |
| | | KGSKPSQQGRYNMTYERFSSSLLILQVREADAAVYYCAVVSGGYNK | |
| | | LIFGAGTRLAVHP | |
| | CDR1-a | VSNAYN | 236 |
| | CDR2-a | GSKP | 237 |
| | CDR3-a | CAVVSGGYNKLIF | 1334 |
| | FWR1-a | KDQVFQPSTVASSEGAVVEIFCNHS | 239 |
| | FWR2-a | FFWYLHFPGCAPRLLVK | 240 |
| | FWR3-a | SQQGRYNMTYERFSSSLLILQVREADAAVYY | 241 |
| | FWR4-a | GAGTRLAVHP | 774 |
| TCRB- | b-Variable | GAAGCTGGAGTTGCCCAGTCTCCCAGATATAAGATTATAGAGAA | 1335 |
| 153 | nucleotide | AAGGCAGAGTGTGGCTTTTTGGTGCAATCCTATATCTGGCCATGC | |
| | | TACCCTTTACTGGTACCAGCAGATCCTGGGACAGGGCCCAAAGCT | |
| | | TCTGATTCAGTTTCAGAATAACGGTGTAGTGGATGATTCACAGTT | |
| | | GCCTAAGGATCGATTTTCTGCAGAGAGGCTCAAAGGAGTAGACT | |
| | | CCACTCTCAAGATCCAACCTGCAAAGCTTGAGGACTCGGCCGTGT | |
| | | ATCTCTGTGCCAGCAGCCTAGGGTACGATACGCAGTATTTTGGCC | |
| | | CAGGCACCCGGCTGACAGTGCTCG | |
| | Variable-b | EAGVAQSPRYKIIEKRQSVAFWCNPISGHATLYWYQQILGQGPKLLI | 1336 |
| | | QFQNNGVVDDSQLPKDRFSAERLKGVDSTLKIQPAKLEDSAVYLCA | |
| | | SSLGYDTQYFGPGTRLTVL | |
| | CDR1-b | SGHAT | 894 |
| | CDR2-b | FQNNGV | 895 |
| | CDR3-b | CASSLGYDTQYF | 1337 |
| | FWR1-b | EAGVAQSPRYKIIEKRQSVAFWCNPI | 897 |
| | FWR2-b | LYWYQQILGQGPKLLIQ | 898 |
| | FWR3-b | VDDSQLPKDRFSAERLKGVDSTLKIQPAKLEDSAVYL | 899 |
| | FWR4-b | GPGTRLTVL | 9 |
| TCRA- | a-Variable | GGACAACAGGTAATGCAAATTCCTCAGTACCAGCATGTACAAGA | 1338 |
| 153 | nucleotide | AGGAGAGGACTTCACCACGTACTGCAATTCCTCAACTACTTTAAG | |
| | | CAATATACAGTGGTATAAGCAAAGGCCTGGTGGACATCCCGTTTT | |
| | | TTTGATACAGTTAGTGAAGAGTGGAGAAGTGAAGAAGCAGAAAA | |
| | | GACTGACATTTCAGTTTGGAGAAGCAAAAAAGAACAGCTCCCTG | |
| | | CACATCACAGCCACCCAGACTACAGATGTAGGAACCTACTTCTGT | |
| | | GCAGGGCGCGGGAACTCTGGGGCTGGGAGTTACCAACTCACTTTC | |
| | | GGGAAGGGGACCAAACTCTCGGTCATACCAA | |
| | Variable-a | GQQVMQIPQYQHVQEGEDFTTYCNSSTTLSNIQWYKQRPGGHPVFLI | 1339 |
| | | QLVKSGEVKKQKRLTFQFGEAKKNSSLHITATQTTDVGTYFCAGRG | |
| | | NSGAGSYQLTFGKGTKLSVIP | |
| | CDR1-a | TTLSN | 575 |
| | CDR2-a | LVKSGEV | 576 |
| | CDR3-a | CAGRGNSGAGSYQLTF | 1340 |
| | FWR1-a | GQQVMQIPQYQHVQEGEDFTTYCNSS | 578 |
| | FWR2-a | IQWYKQRPGGHPVFLIQ | 579 |
| | FWR3-a | KKQKRLTFQFGEAKKNSSLHITATQTTDVGTYF | 580 |
| | FWR4-a | GKGTKLSVIP | 489 |
| TCRB- | b-Variable | AAGGCTGGAGTCACTCAAACTCCAAGATATCTGATCAAAACGAG | 1341 |
| 154 | nucleotide | AGGACAGCAAGTGACACTGAGCTGCTCCCCTATCTCTGGGCATAG | |
| | | GAGTGTATCCTGGTACCAACAGACCCCAGGACAGGGCCTTCAGTT | |
| | | CCTCTTTGAATACTTCAGTGAGACACAGAGAAACAAAGGAAACT | |
| | | TCCCTGGTCGATTCTCAGGGCGCCAGTTCTCTAACTCTCGCTCTGA | |
| | | GATGAATGTGAGCACCTTGGAGCTGGGGGACTCGGCCCTTTATCT | |
| | | TTGCGCCAGCAGTCCCAGGGGCTACAATGAGCAGTTCTTCGGGCC | |
| | | AGGGACACGGCTCACCGTGCTAG | |
| | Variable-b | KAGVTQTPRYLIKTRGQQVTLSCSPISGHRSVSWYQQTPGQGLQFLF | 1342 |
| | | EYFSETQRNKGNFPGRFSGRQFSNSRSEMNVSTLELGDSALYLCASS | |
| | | PRGYNEQFFGPGTRLTVL | |
| | CDR1-b | SGHRS | 514 |
| | CDR2-b | YFSETQ | 515 |
| | CDR3-b | CASSPRGYNEQFF | 1343 |
| | FWR1-b | KAGVTQTPRYLIKTRGQQVTLSCSPI | 517 |
| | FWR2-b | VSWYQQTPGQGLQFLFE | 518 |
| | FWR3-b | RNKGNFPGRFSGRQFSNSRSEMNVSTLELGDSALYL | 519 |
| | FWR4-b | GPGTRLTVL | 9 |
| TCRA- | a-Variable | AAACAGGAGGTGACGCAGATTCCTGCAGCTCTGAGTGTCCCAGA | 1344 |
| 154.1 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGCCGCATACTCTGGGGCTGGGAGTTACCAACTCACTTTCGG | |
| | | GAAGGGGACCAAACTCTCGGTCATACCAA | |
| | Variable-a | KQEVTQIPAALSVPEGENLVLNCSFTDSAIYNLQWFRQDPGKGLTSL | 1345 |
| | | LLIQSSQREQTSGRLNASLDKSSGRSTLYIAASQPGDSATYLCAAAYS | |
| | | GAGSYQLTFGKGTKLSVIP | |
| | CDR1-a | DSAIYN | 118 |
| | CDR2-a | IQSSQRE | 119 |
| | CDR3-a | CAAAYSGAGSYQLTF | 1346 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GKGTKLSVIP | 489 |
| TCRA- | a-Variable | AGTCAACAGGGAGAAGAGGATCCTCAGGCCTTGAGCATCCAGGA | 1347 |
| 154.2 | nucleotide | GGGTGAAAATGCCACCATGAACTGCAGTTACAAAACTAGTATAA | |
| | | ACAATTTACAGTGGTATAGACAAAATTCAGGTAGAGGCCTTGTCC | |
| | | ACCTAATTTTAATACGTTCAAATGAAAGAGAGAAACACAGTGGA | |
| | | AGATTAAGAGTCACGCTTGACACTTCCAAGAAAAGCAGTTCCTTG | |
| | | TTGATCACGGCTTCCCGGGCAGCAGACACTGCTTCTTACTTCTGT | |
| | | GCTACGGACGCCCCCCTAAACGACTACAAGCTCAGCTTTGGAGCC | |
| | | GGAACCACAGTAACTGTAAGAGCAA | |
| | Variable-a | SQQGEEDPQALSIQEGENATMNCSYKTSINNLQWYRQNSGRGLVHL | 1348 |
| | | ILIRSNEREKHSGRLRVTLDTSKKSSSLLITASRAADTASYFCATDAPL | |
| | | NDYKLSFGAGTTVTVRA | |
| | CDR1-a | TSINN | 552 |
| | CDR2-a | IRSNERE | 553 |
| | CDR3-a | CATDAPLNDYKLSF | 1349 |
| | FWR1-a | SQQGEEDPQALSIQEGENATMNCSYK | 555 |
| | FWR2-a | LQWYRQNSGRGLVHLIL | 556 |
| | FWR3-a | KHSGRLRVTLDTSKKSSSLLITASRAADTASYF | 557 |
| | FWR4-a | GAGTTVTVRA | 1181 |
| TCRB- | b-Variable | GAAGCCCAAGTGACCCAGAACCCAAGATACCTCATCACAGTGAC | 501 |
| 155 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTCCTCACGCCGGGGAGCTGTTTTTTGGAGAAG | |
| | | GCTCTAGGCTGACCGTACTGG | |
| | Variable-b | EAQVTQNPRYLITVTGKKLTVTCSQNMNHEYMSWYRQDPGLGLRQ | 502 |
| | | IYYSMNVEVTDKGDVPEGYKVSRKEKRNFPLILESPSPNQTSLYFCA | |
| | | SSPHAGELFFGEGSRLTVL | |
| | CDR1-b | MNHEY | 252 |
| | CDR2-b | SMNVEV | 366 |
| | CDR3-b | CASSPHAGELFF | 503 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GEGSRLTVL | 63 |
| TCRA- | a-Variable | GGAATACAAGTGGAGCAGAGTCCTCCAGACCTGATTCTCCAGGA | 504 |
| 155 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTCCCA | |
| | | CCGATGACAACTGACAGCTGGGGGAAATTGCAGTTTGGAGCAGG | |
| | | GACCCAGGTTGTGGTCACCCCAG | |
| | Variable-a | GIQVEQSPPDLILQEGANSTLRCNFSDSVNNLQWFHQNPWGQLINLF | 505 |
| | | YIPSGTKQNGRLSATTVATERYSLLYISSSQTTDSGVYFCAVPPMTTD | |
| | | SWGKLQFGAGTQVVVTP | |
| | CDR1-a | DSVNN | 506 |
| | CDR2-a | IPSGT | 507 |
| | CDR3-a | CAVPPMTTDSWGKLQF | 508 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GAGTQVVVTP | 54 |
| TCRB- | b-Variable | AAGGCTGGAGTCACTCAAACTCCAAGATATCTGATCAAAACGAG | 512 |
| 156 | nucleotide | AGGACAGCAAGTGACACTGAGCTGCTCCCCTATCTCTGGGCATAG | |
| | | GAGTGTATCCTGGTACCAACAGACCCCAGGACAGGGCCTTCAGTT | |
| | | CCTCTTTGAATACTTCAGTGAGACACAGAGAAACAAAGGAAACT | |
| | | TCCCTGGTCGATTCTCAGGGCGCCAGTTCTCTAACTCTCGCTCTGA | |
| | | GATGAATGTGAGCACCTTGGAGCTGGGGGACTCGGCCCTTTATCT | |
| | | TTGCGCCAGCAGCTTACGGCGGGGGCAATCCTACGAGCAGTACTT | |
| | | CGGGCCGGGCACCAGGCTCACGGTCACAG | |
| | Variable-b | KAGVTQTPRYLIKTRGQQVTLSCSPISGHRSVSWYQQTPGQGLQFLF | 513 |
| | | EYFSETQRNKGNFPGRFSGRQFSNSRSEMNVSTLELGDSALYLCASS | |
| | | LRRGQSYEQYFGPGTRLTVT | |
| | CDR1-b | SGHRS | 514 |
| | CDR2-b | YFSETQ | 515 |
| | CDR3-b | CASSLRRGQSYEQYF | 516 |
| | FWR1-b | KAGVTQTPRYLIKTRGQQVTLSCSPI | 517 |
| | FWR2-b | VSWYQQTPGQGLQFLFE | 518 |
| | FWR3-b | RNKGNFPGRFSGRQFSNSRSEMNVSTLELGDSALYL | 519 |
| | FWR4-b | GPGTRLTVT | 85 |
| TCRA- | a-Variable | GATGCTAAGACCACACAGCCAAATTCAATGGAGAGTAACGAAGA | 520 |
| 156 | nucleotide | AGAGCCTGTTCACTTGCCTTGTAACCACTCCACAATCAGTGGAAC | |
| | | TGATTACATACATTGGTATCGACAGCTTCCCTCCCAGGGTCCAGA | |
| | | GTACGTGATTCATGGTCTTACAAGCAATGTGAACAACAGAATGGC | |
| | | CTCTCTGGCAATCGCTGAAGACAGAAAGTCCAGTACCTTGATCCT | |
| | | GCACCGTGCTACCTTGAGAGATGCTGCTGTGTACTACTGCATCCT | |
| | | GGATGCAGGCAACATGCTCACCTTTGGAGGGGGAACAAGGTTAA | |
| | | TGGTCAAACCCC | |
| | Variable-a | DAKTTQPNSMESNEEEPVHLPCNHSTISGTDYIHWYRQLPSQGPEYV | 521 |
| | | IHGLTSNVNNRMASLAIAEDRKSSTLILHRATLRDAAVYYCILDAGN | |
| | | MLTFGGGTRLMVKP | |
| | CDR1-a | TISGTDY | 153 |
| | CDR2-a | GLTSN | 154 |
| | CDR3-a | CILDAGNMLTF | 522 |
| | FWR1-a | DAKTTQPNSMESNEEEPVHLPCNHS | 156 |
| | FWR2-a | IHWYRQLPSQGPEYVIH | 157 |
| | FWR3-a | VNNRMASLAIAEDRKSSTLILHRATLRDAAVYY | 158 |
| | FWR4-a | GGGTRLMVKP | 141 |
| TCRB- | b-Variable | GAACCTGAAGTCACCCAGACTCCCAGCCATCAGGTCACACAGAT | 523 |
| 157 | nucleotide | GGGACAGGAAGTGATCTTGCGCTGTGTCCCCATCTCTAATCACTT | |
| | | ATACTTCTATTGGTACAGACAAATCTTGGGGCAGAAAGTCGAGTT | |
| | | TCTGGTTTCCTTTTATAATAATGAAATCTCAGAGAAGTCTGAAAT | |
| | | ATTCGATGATCAATTCTCAGTTGAAAGGCCTGATGGATCAAATTT | |
| | | CACTCTGAAGATCCGGTCCACAAAGCTGGAGGACTCAGCCATGT | |
| | | ACTTCTGTGCCAGCAAGGAGGGTTGGGGGGACACTGAAGCTTTCT | |
| | | TTGGACAAGGCACCAGACTCACAGTTGTAG | |
| | Variable-b | EPEVTQTPSHQVTQMGQEVILRCVPISNHLYFYWYRQILGQKVEFLV | 524 |
| | | SFYNNEISEKSEIFDDQFSVERPDGSNFTLKIRSTKLEDSAMYFCASKE | |
| | | GWGDTEAFFGQGTRLTVV | |
| | CDR1-b | SNHLY | 525 |
| | CDR2-b | FYNNEI | 526 |
| | CDR3-b | CASKEGWGDTEAFF | 527 |
| | FWR1-b | EPEVTQTPSHQVTQMGQEVILRCVPI | 528 |
| | FWR2-b | FYWYRQILGQKVEFLVS | 529 |
| | FWR3-b | SEKSEIFDDQFSVERPDGSNFTLKIRSTKLEDSAMYF | 530 |
| | FWR4-b | GQGTRLTVV | 246 |
| TCRA- | a-Variable | ACCCAGCTGCTGGAGCAGAGCCCTCAGTTTCTAAGCATCCAAGAG | 531 |
| 157 | nucleotide | GGAGAAAATCTCACTGTGTACTGCAACTCCTCAAGTGTTTTTTCC | |
| | | AGCTTACAATGGTACAGACAGGAGCCTGGGGAAGGTCCTGTCCT | |
| | | CCTGGTGACAGTAGTTACGGGTGGAGAAGTGAAGAAGCTGAAGA | |
| | | GACTAACCTTTCAGTTTGGTGATGCAAGAAAGGACAGTTCTCTCC | |
| | | ACATCACTGCGGCCCAGCCTGGTGATACAGGCCTCTACCTCTGTG | |
| | | CAGGAGTAGGAATGGATAGCAGCTATAAATTGATCTTCGGGAGT | |
| | | GGGACCAGACTGCTGGTCAGGCCTG | |
| | Variable-a | TQLLEQSPQFLSIQEGENLTVYCNSSSVFSSLQWYRQEPGEGPVLLVT | 532 |
| | | VVTGGEVKKLKRLTFQFGDARKDSSLHITAAQPGDTGLYLCAGVG | |
| | | MDSSYKLIFGSGTRLL VRP | |
| | CDR1-a | SVFSS | 533 |
| | CDR2-a | VVTGGEV | 534 |
| | CDR3-a | CAGVGMDSSYKLIF | 535 |
| | FWR1-a | TQLLEQSPQFLSIQEGENLTVYCNSS | 536 |
| | FWR2-a | LQWYRQEPGEGPVLLVT | 537 |
| | FWR3-a | KKLKRLTFQFGDARKDSSLHITAAQPGDTGLYL | 538 |
| | FWR4-a | GSGTRLLVRP | 539 |
| TCRB- | b-Variable | AATGCTGGTGTCACTCAGACCCCAAAATTCCAGGTCCTGAAGACA | 540 |
| 158 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTTCCCGACAGGGTTATCAGCCCCAGCATTTTGG | |
| | | TGATGGGACTCGACTCTCCATCCTAG | |
| | Variable-b | NAGVTQTPKFQVLKTGQSMTLQCAQDMNHEYMSWYRQDPGMGLR | 541 |
| | | LIHYSVGAGITDQGEVPNGYNVSRSTTEDFPLRLLSAAPSQTSVYFCA | |
| | | SSSRQGYQPQHFGDGTRLSIL | |
| | CDR1-b | MNHEY | 252 |
| | CDR2-b | SVGAGI | 253 |
| | CDR3-b | CASSSRQGYQPQHF | 542 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GDGTRLSIL | 27 |
| TCRA- | a-Variable | GCTCAGACAGTCACTCAGTCTCAACCAGAGATGTCTGTGCAGGAG | 543 |
| 158 | nucleotide | GCAGAGACCGTGACCCTGAGCTGCACATATGACACCAGTGAGAG | |
| | | TGATTATTATTTATTCTGGTACAAGCAGCCTCCCAGCAGGCAGAT | |
| | | GATTCTCGTTATTCGCCAAGAAGCTTATAAGCAACAGAATGCAAC | |
| | | AGAGAATCGTTTCTCTGTGAACTTCCAGAAAGCAGCCAAATCCTT | |
| | | CAGTCTCAAGATCTCAGACTCACAGCTGGGGGATGCCGCGATGTA | |
| | | TTTCTGTGCTTATAGGAGCGGAAACACACCTCTTGTCTTTGGAAA | |
| | | GGGCACAAGACTTTCTGTGATTGCAA | |
| | Variable-a | AQTVTQSQPEMSVQEAETVTLSCTYDTSESDYYLFWYKQPPSRQMI | 544 |
| | | LVIRQEAYKQQNATENRFSVNFQKAAKSFSLKISDSQLGDAAMYFC | |
| | | AYRSGNTPLVFGKGTRLSVIA | |
| | CDR1-a | TSESDYY | 286 |
| | CDR2-a | QEAYKQQN | 287 |
| | CDR3-a | CAYRSGNTPLVF | 545 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDAAMYF | 291 |
| | FWR4-a | GKGTRLSVIA | 546 |
| TCRB- | b-Variable | AATGCTGGTGTCACTCAGACCCCAAAATTCCAGGTCCTGAAGACA | 547 |
| 159 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTTACTCGCTGCTGTCGCCCTCCTACGAGCAGTA | |
| | | CTTCGGGCCGGGCACCAGGCTCACGGTCACAG | |
| | Variable-b | NAGVTQTPKFQVLKTGQSMTLQCAQDMNHEYMSWYRQDPGMGLR | 548 |
| | | LIHYSVGAGITDQGEVPNGYNVSRSTTEDFPLRLLSAAPSQTSVYFCA | |
| | | SSYSLLSPSYEQYFGPGTRLTVT | |
| | CDR1-b | MNHEY | 252 |
| | CDR2-b | SVGAGI | 253 |
| | CDR3-b | CASSYSLLSPSYEQYF | 549 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GPGTRLTVT | 85 |
| TCRA- | a-Variable | AGTCAACAGGGAGAAGAGGATCCTCAGGCCTTGAGCATCCAGGA | 550 |
| 159 | nucleotide | GGGTGAAAATGCCACCATGAACTGCAGTTACAAAACTAGTATAA | |
| | | ACAATTTACAGTGGTATAGACAAAATTCAGGTAGAGGCCTTGTCC | |
| | | ACCTAATTTTAATACGTTCAAATGAAAGAGAGAAACACAGTGGA | |
| | | AGATTAAGAGTCACGCTTGACACTTCCAAGAAAAGCAGTTCCTTG | |
| | | TTGATCACGGCTTCCCGGGCAGCAGACACTGCTTCTTACTTCTGT | |
| | | GCTACGGACCACCGAACTAGCTATGGAAAGCTGACATTTGGACA | |
| | | AGGGACCATCTTGACTGTCCATCCAA | |
| | Variable-a | SQQGEEDPQALSIQEGENATMNCSYKTSINNLQWYRQNSGRGLVHL | 551 |
| | | ILIRSNEREKHSGRLRVTLDTSKKSSSLLITASRAADTASYFCATDHR | |
| | | TSYGKLTFGQGTILTVHP | |
| | CDR1-a | TSINN | 552 |
| | CDR2-a | IRSNERE | 553 |
| | CDR3-a | CATDHRTSYGKLTF | 554 |
| | FWR1-a | SQQGEEDPQALSIQEGENATMNCSYK | 555 |
| | FWR2-a | LQWYRQNSGRGLVHLIL | 556 |
| | FWR3-a | KHSGRLRVTLDTSKKSSSLLITASRAADTASYF | 557 |
| | FWR4-a | GQGTILTVHP | 76 |
| TCRB- | b-Variable | GAGACTGGAGTCACCCAAAGTCCCACACACCTGATCAAAACGAG | 558 |
| 160 | nucleotide | AGGACAGCAAGTGACTCTGAGATGCTCTTCTCAGTCTGGGCACAA | |
| | | CACTGTGTCCTGGTACCAACAGGCCCTGGGTCAGGGGCCCCAGTT | |
| | | TATCTTTCAGTATTATAGGGAGGAAGAGAATGGCAGAGGAAACT | |
| | | TCCCTCCTAGATTCTCAGGTCTCCAGTTCCCTAATTATAGCTCTGA | |
| | | GCTGAATGTGAACGCCTTGGAGCTGGACGACTCGGCCCTGTATCT | |
| | | CTGTGCCAGCAGCTCAGAACGGGGACATAAGGGCTACACCTTCG | |
| | | GTTCGGGGACCAGGTTAACCGTTGTAG | |
| | Variable-b | ETGVTQSPTHLIKTRGQQVTLRCSSQSGHNTVSWYQQALGQGPQFIF | 559 |
| | | QYYREEENGRGNFPPRFSGLQFPNYSSELNVNALELDDSALYLCASS | |
| | | SERGHKGYTFGSGTRLTVV | |
| | CDR1-b | SGHNT | 127 |
| | CDR2-b | YYREEE | 128 |
| | CDR3-b | CASSSERGHKGYTF | 560 |
| | FWR1-b | ETGVTQSPTHLIKTRGQQVTLRCSSQ | 130 |
| | FWR2-b | VSWYQQALGQGPQFIFQ | 131 |
| | FWR3-b | NGRGNFPPRFSGLQFPNYSSELNVNALELDDSALYL | 132 |
| | FWR4-b | GSGTRLTVV | 103 |
| TCRA- | a-Variable | AAAAACCAAGTGGAGCAGAGTCCTCAGTCCCTGATCATCCTGGA | 561 |
| 160 | nucleotide | GGGAAAGAACTGCACTCTTCAATGCAATTATACAGTGAGCCCCTT | |
| | | CAGCAACTTAAGGTGGTATAAGCAAGATACTGGGAGAGGTCCTG | |
| | | TTTCCCTGACAATCATGACTTTCAGTGAGAACACAAAGTCGAACG | |
| | | GAAGATATACAGCAACTCTGGATGCAGACACAAAGCAAAGCTCT | |
| | | CTGCACATCACAGCCTCCCAGCTCAGCGATTCAGCCTCCTACATC | |
| | | TGTGTGTTATCCGGTGGTGCTACAAACAAGCTCATCTTTGGAACT | |
| | | GGCACTCTGCTTGCTGTCCAGCCAA | |
| | Variable-a | KNQVEQSPQSLIILEGKNCTLQCNYTVSPFSNLRWYKQDTGRGPVSL | 562 |
| | | TIMTFSENTKSNGRYTATLDADTKQSSLHITASQLSDSASYICVLSGG | |
| | | ATNKLIFGTGTLLAVQP | |
| | CDR1-a | VSPFSN | 135 |
| | CDR2-a | MTFSENT | 136 |
| | CDR3-a | CVLSGGATNKLIF | 563 |
| | FWR1-a | KNQVEQSPQSLIILEGKNCTLQCNYT | 138 |
| | FWR2-a | LRWYKQDTGRGPVSLTI | 139 |
| | FWR3-a | KSNGRYTATLDADTKQSSLHITASQLSDSASYI | 140 |
| | FWR4-a | GTGTLLAVQP | 480 |
| TCRB- | b-Variable | GATGGTGGAATCACTCAGTCCCCAAAGTACCTGTTCAGAAAGGA | 564 |
| 161 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGTACGACTAGCGGGAGTTTGGAGACCCAGTA | |
| | | CTTCGGGCCAGGCACGCGGCTCCTGGTGCTCG | |
| | Variable-b | DGGITQSPKYLFRKEGQNVTLSCEQNLNHDAMYWYRQDPGQGLRLI | 565 |
| | | YYSQIVNDFQKGDIAEGYSVSREKKESFPLTVTSAQKNPTAFYLCAS | |
| | | STTSGSLETQYFGPGTRLLVL | |
| | CDR1-b | LNHDA | 216 |
| | CDR2-b | SQIVND | 217 |
| | CDR3-b | CASSTTSGSLETQYF | 566 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLLVL | 67 |
| TCRA- | a-Variable | AAAAACCAAGTGGAGCAGAGTCCTCAGTCCCTGATCATCCTGGA | 567 |
| 161 | nucleotide | GGGAAAGAACTGCACTCTTCAATGCAATTATACAGTGAGCCCCTT | |
| | | CAGCAACTTAAGGTGGTATAAGCAAGATACTGGGAGAGGTCCTG | |
| | | TTTCCCTGACAATCATGACTTTCAGTGAGAACACAAAGTCGAACG | |
| | | GAAGATATACAGCAACTCTGGATGCAGACACAAAGCAAAGCTCT | |
| | | CTGCACATCACAGCCTCCCAGCTCAGCGATTCAGCCTCCTACATC | |
| | | TGTGTGGTGAGCGCGGGCAGGGAATATGGAAACAAGCTGGTCTT | |
| | | TGGCGCAGGAACCATTCTGAGAGTCAAGTCCT | |
| | Variable-a | KNQVEQSPQSLIILEGKNCTLQCNYTVSPFSNLRWYKQDTGRGPVSL | 568 |
| | | TIMTFSENTKSNGRYTATLDADTKQSSLHITASQLSDSASYICVVSAG | |
| | | REYGNKLVFGAGTILRVKS | |
| | CDR1-a | VSPFSN | 135 |
| | CDR2-a | MTFSENT | 136 |
| | CDR3-a | CVVSAGREYGNKLVF | 569 |
| | FWR1-a | KNQVEQSPQSLIILEGKNCTLQCNYT | 138 |
| | FWR2-a | LRWYKQDTGRGPVSLTI | 139 |
| | FWR3-a | KSNGRYTATLDADTKQSSLHITASQLSDSASYI | 140 |
| | FWR4-a | GAGTILRVKS | 18 |
| TCRB- | b-Variable | GAAGCCCAAGTGACCCAGAACCCAAGATACCTCATCACAGTGAC | 570 |
| 162 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTTCGACAGGCATGAACACTGAAGCTTTCTTTG | |
| | | GACAAGGCACCAGACTCACAGTTGTAG | |
| | Variable-b | EAQVTQNPRYLITVTGKKLTVTCSQNMNHEYMSWYRQDPGLGLRQ | 571 |
| | | IYYSMNVEVTDKGDVPEGYKVSRKEKRNFPLILESPSPNQTSLYFCA | |
| | | SSSTGMNTEAFFGQGTRLTVV | |
| | CDR1-b | MNHEY | 252 |
| | CDR2-b | SMNVEV | 366 |
| | CDR3-b | CASSSTGMNTEAFF | 572 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GQGTRLTVV | 246 |
| TCRA- | a-Variable | GGACAACAGGTAATGCAAATTCCTCAGTACCAGCATGTACAAGA | 573 |
| 162 | nucleotide | AGGAGAAGACTTCACCACGTACTGCAATTCCTCAACTACTTTAAG | |
| | | CAATATACAGTGGTATAAGCAAAGGCCTGGTGGACATCCCGTTTT | |
| | | TTTGATACAGTTAGTGAAGAGTGGAGAAGTGAAGAAGCAGAAAA | |
| | | GACTGACATTTCAGTTTGGAGAAGCAAAAAAGAACAGCTCCCTG | |
| | | CACATCACAGCCACCCAGACTACAGATGTAGGAACCTACTTCTGT | |
| | | GCAGGGCTAACTCGGGATAGCAACTATCAGTTAATCTGGGGCGCT | |
| | | GGGACCAAGCTAATTATAAAGCCAG | |
| | Variable-a | GQQVMQIPQYQHVQEGEDFTTYCNSSTTLSNIQWYKQRPGGHPVFLI | 574 |
| | | QLVKSGEVKKQKRLTFQFGEAKKNSSLHITATQTTDVGTYFCAGLT | |
| | | RDSNYQLIWGAGTKLIIKP | |
| | CDR1-a | TTLSN | 575 |
| | CDR2-a | LVKSGEV | 576 |
| | CDR3-a | CAGLTRDSNYQLIW | 577 |
| | FWR1-a | GQQVMQIPQYQHVQEGEDFTTYCNSS | 578 |
| | FWR2-a | IQWYKQRPGGHPVFLIQ | 579 |
| | FWR3-a | KKQKRLTFQFGEAKKNSSLHITATQTTDVGTYF | 580 |
| | FWR4-a | GAGTKLIIKP | 356 |
| TCRB- | b-Variable | GATGGTGGAATCACTCAGTCCCCAAAGTACCTGTTCAGAAAGGA | 581 |
| 163 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGTTCGTCCCCCTTTGGCTACACCTTCGGTTCG | |
| | | GGGACCAGGTTAACCGTTGTAG | |
| | Variable-b | DGGITQSPKYLFRKEGQNVTLSCEQNLNHDAMYWYRQDPGQGLRLI | 582 |
| | | YYSQIVNDFQKGDIAEGYSVSREKKESFPLTVTSAQKNPTAFYLCAS | |
| | | SSSPFGYTFGSGTRLTVV | |
| | CDR1-b | LNHDA | 216 |
| | CDR2-b | SQIVND | 217 |
| | CDR3-b | CASSSSPFGYTF | 583 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GSGTRLTVV | 103 |
| TCRA- | a-Variable | AAACAGGAGGTGACGCAGATTCCTGCAGCTCTGAGTGTCCCAGA | 584 |
| 163 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGAGCGATACCTCAGGAACCTACAAATACATCTTTGGAAC | |
| | | AGGCACCAGGCTGAAGGTTTTAGCAA | |
| | Variable-a | KQEVTQIPAALSVPEGENLVLNCSFTDSAIYNLQWFRQDPGKGLTSL | 585 |
| | | LLIQSSQREQTSGRLNASLDKSSGRSTLYIAASQPGDSATYLCAVSDT | |
| | | SGTYKYIFGTGTRLKVLA | |
| | CDR1-a | DSAIYN | 118 |
| | CDR2-a | IQSSQRE | 119 |
| | CDR3-a | CAVSDTSGTYKYIF | 586 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GTGTRLKVLA | 587 |
| TCRB- | b-Variable | GATACTGGAGTCTCCCAGGACCCCAGACACAAGATCACAAAGAG | 588 |
| 164 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCCACGTCGATGGGGGCGGTGAGACCCAG | |
| | | TACTTCGGGCCAGGCACGCGGCTCCTGGTGCTCG | |
| | Variable-b | DTGVSQDPRHKITKRGQNVTFRCDPISEHNRLYWYRQTLGQGPEFLT | 589 |
| | | YFQNEAQLEKSRLLSDRFSAERPKGSFSTLEIQRTEQGDSAMYLCASS | |
| | | HVDGGGETQYFGPGTRLLVL | |
| | CDR1-b | SEHNR | 442 |
| | CDR2-b | FQNEAQ | 443 |
| | CDR3-b | CASSHVDGGGETQYF | 590 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GPGTRLLVL | 67 |
| TCRA- | a-Variable | CTTGCTAAGACCACCCAGCCCATCTCCATGGACTCATATGAAGGA | 592 |
| 164 | nucleotide | CAAGAAGTGAACATAACCTGTAGCCACAACAACATTGCTACAAA | |
| | | TGATTATATCACGTGGTACCAACAGTTTCCCAGCCAAGGACCACG | |
| | | ATTTATTATTCAAGGATACAAGACAAAAGTTACAAACGAAGTGG | |
| | | CCTCCCTGTTTATCCCTGCCGACAGAAAGTCCAGCACTCTGAGCC | |
| | | TGCCCCGGGTTTCCCTGAGCGACACTGCTGTGTACTACTGCCTCG | |
| | | CTACAGGAGGAAGCTACATACCTACATTTGGAAGAGGAACCAGC | |
| | | CTTATTGTTCATCCGT | |
| | Variable-a | LAKTTQPISMDSYEGQEVNITCSHNNIATNDYITWYQQFPSQGPRFII | 593 |
| | | QGYKTKVTNEVASLFIPADRKSSTLSLPRVSLSDTAVYYCLATGGSYI | |
| | | PTFGRGTSLIVHP | |
| | CDR1-a | NIATNDY | 594 |
| | CDR2-a | GYKTK | 595 |
| | CDR3-a | CLATGGSYIPTF | 596 |
| | FWR1-a | LAKTTQPISMDSYEGQEVNITCSHN | 597 |
| | FWR2-a | ITWYQQFPSQGPRFIIQ | 598 |
| | FWR3-a | VTNEVASLFIPADRKSSTLSLPRVSLSDTAVYY | 599 |
| | FWR4-a | GRGTSLIVHP | 600 |
| TCRB- | b-Variable | AGTGCTGTCATCTCTCAAAAGCCAAGCAGGGATATCTGTCAACGT | 601 |
| 165 | nucleotide | GGAACCTCCCTGACGATCCAGTGTCAAGTCGATAGCCAAGTCACC | |
| | | ATGATGTTCTGGTACCGTCAGCAACCTGGACAGAGCCTGACACTG | |
| | | ATCGCAACTGCAAATCAGGGCTCTGAGGCCACATATGAGAGTGG | |
| | | ATTTGTCATTGACAAGTTTCCCATCAGCCGCCCAAACCTAACATT | |
| | | CTCAACTCTGACTGTGAGCAACATGAGCCCTGAAGACAGCAGCA | |
| | | TATATCTCTGCAGCGTTGAAATGGGGCGGAGCTCCTACAATGAGC | |
| | | AGTTCTTCGGGCCAGGGACACGGCTCACCGTGCTAG | |
| | Variable-b | SAVISQKPSRDICQRGTSLTIQCQVDSQVTMMFWYRQQPGQSLTLIA | 602 |
| | | TANQGSEATYESGFVIDKFPISRPNLTFSTLTVSNMSPEDSSIYLCSVE | |
| | | MGRSSYNEQFFGPGTRLTVL | |
| | CDR1-b | SQVTM | 97 |
| | CDR2-b | ANQGSEA | 98 |
| | CDR3-b | CSVEMGRSSYNEQFF | 603 |
| | FWR1-b | SAVISQKPSRDICQRGTSLTIQCQVD | 100 |
| | FWR2-b | MFWYRQQPGQSLTLIAT | 101 |
| | FWR3-b | TYESGFVIDKFPISRPNLTFSTLTVSNMSPEDSSIYL | 102 |
| | FWR4-b | GPGTRLTVL | 9 |
| TCRA- | a-Variable | GGACAACAGGTAATGCAAATTCCTCAGTACCAGCATGTACAAGA | 604 |
| 165 | nucleotide | AGGAGAAGACTTCACCACGTACTGCAATTCCTCAACTACTTTAAG | |
| | | CAATATACAGTGGTATAAGCAAAGGCCTGGTGGACATCCCGTTTT | |
| | | TTTGATACAGTTAGTGAAGAGTGGAGAAGTGAAGAAGCAGAAAA | |
| | | GACTGACATTTCAGTTTGGAGAAGCAAAAAAGAACAGCTCCCTG | |
| | | CACATCACAGCCACCCAGACTACAGATGTAGGAACCTACTTCTGT | |
| | | GCAGGGACGTATGACAAGATCATCTTTGGAAAAGGGACACGACT | |
| | | TCATATTCTCCCCA | |
| | Variable-a | GQQVMQIPQYQHVQEGEDFTTYCNSSTTLSNIQWYKQRPGGHPVFLI | 605 |
| | | QLVKSGEVKKQKRLTFQFGEAKKNSSLHITATQTTDVGTYFCAGTY | |
| | | DKIIFGKGTRLHILP | |
| | CDR1-a | TTLSN | 575 |
| | CDR2-a | LVKSGEV | 576 |
| | CDR3-a | CAGTYDKIIF | 606 |
| | FWR1-a | GQQVMQIPQYQHVQEGEDFTTYCNSS | 578 |
| | FWR2-a | IQWYKQRPGGHPVFLIQ | 579 |
| | FWR3-a | KKQKRLTFQFGEAKKNSSLHITATQTTDVGTYF | 580 |
| | FWR4-a | GKGTRLHILP | 607 |
| TCRB- | b-Variable | GAAGCCCAAGTGACCCAGAACCCAAGATACCTCATCACAGTGAC | 608 |
| 166 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTTTATCAACCCCCTACGAGCAGTACTTCGGGC | |
| | | CGGGCACCAGGCTCACGGTCACAG | |
| | Variable-b | EAQVTQNPRYLITVTGKKLTVTCSQNMNHEYMSWYRQDPGLGLRQ | 609 |
| | | IYYSMNVEVTDKGDVPEGYKVSRKEKRNFPLILESPSPNQTSLYFCA | |
| | | SSLSTPYEQYFGPGTRLTVT | |
| | CDR1-b | MNHEY | 252 |
| | CDR2-b | SMNVEV | 366 |
| | CDR3-b | CASSLSTPYEQYF | 610 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GPGTRLTVT | 85 |
| TCRA- | a-Variable | GACCAGCAAGTTAAGCAAAATTCACCATCCCTGAGCGTCCAGGA | 611 |
| 166 | nucleotide | AGGAAGAATTTCTATTCTGAACTGTGACTATACTAACAGCATGTT | |
| | | TGATTATTTCCTATGGTACAAAAAATACCCTGCTGAAGGTCCTAC | |
| | | ATTCCTGATATCTATAAGTTCCATTAAGGATAAAAATGAAGATGG | |
| | | AAGATTCACTGTTTTCTTAAACAAAAGTGCCAAGCACCTCTCTCT | |
| | | GCACATTGTGCCCTCCCAGCCTGGAGACTCTGCAGTGTACTTCTG | |
| | | TGCAGCAAGCGGGGTGGCAGGTGAATATGGAAACAAGCTGGTCT | |
| | | TTGGCGCAGGAACCATTCTGAGAGTCAAGTCCT | |
| | Variable-a | DQQVKQNSPSLSVQEGRISILNCDYTNSMFDYFLWYKKYPAEGPTFL | 612 |
| | | ISISSIKDKNEDGRFTVFLNKSAKHLSLHIVPSQPGDSAVYFCAASGV | |
| | | AGEYGNKLVFGAGTILRVKS | |
| | CDR1-a | NSMFDY | 303 |
| | CDR2-a | ISSIKDK | 304 |
| | CDR3-a | CAASGVAGEYGNKLVF | 613 |
| | FWR1-a | DQQVKQNSPSLSVQEGRISILNCDYT | 306 |
| | FWR2-a | FLWYKKYPAEGPTFLIS | 307 |
| | FWR3-a | NEDGRFTVFLNKSAKHLSLHIVPSQPGDSAVYF | 308 |
| | FWR4-a | GAGTILRVKS | 18 |
| TCRB- | b-Variable | ACTGCTGGGATCACCCAGGCACCAACATCTCAGATCCTGGCAGCA | 614 |
| 167 | nucleotide | GGACGGAGCATGACACTGAGATGTACCCAGGATATGAGACATAA | |
| | | TGCCATGTACTGGTATAGACAAGATCTAGGACTGGGGCTAAGGCT | |
| | | CATCCATTATTCAAATACTGCAGGTACCACTGGCAAAGGAGAAGT | |
| | | CCCTGATGGTTATAGTGTCTCCAGAGCAAACACAGATGATTTCCC | |
| | | CCTCACGTTGGCGTCTGCTGTACCCTCTCAGACATCTGTGTACTTC | |
| | | TGTGCCAGCAGCGCGGGACAGGGGGCCGGCGATGAGCAGTTCTT | |
| | | CGGGCCAGGGACACGGCTCACCGTGCTAG | |
| | Variable-b | TAGITQAPTSQILAAGRSMTLRCTQDMRHNAMYWYRQDLGLGLRLI | 615 |
| | | HYSNTAGTTGKGEVPDGYSVSRANTDDFPLTLASAVPSQTSVYFCAS | |
| | | SAGQGAGDEQFFGPGTRLTVL | |
| | CDR1-b | MRHNA | 295 |
| | CDR2-b | SNTAGT | 296 |
| | CDR3-b | CASSAGQGAGDEQFF | 616 |
| | FWR1-b | TAGITQAPTSQILAAGRSMTLRCTQD | 298 |
| | FWR2-b | MYWYRQDLGLGLRLIHY | 299 |
| | FWR3-b | TGKGEVPDGYSVSRANTDDFPLTLASA VPSQTSVYF | 300 |
| | FWR4-b | GPGTRLTVL | 9 |
| TCRA- | a-Variable | GGACAAAACATTGACCAGCCCACTGAGATGACAGCTACGGAAGG | 617 |
| 167 | nucleotide | TGCCATTGTCCAGATCAACTGCACGTACCAGACATCTGGGTTCAA | |
| | | CGGGCTGTTCTGGTACCAGCAACATGCTGGCGAAGCACCCACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTG | |
| | | AGATCCCCGCATGGCTCTGGGGCTGGGAGTTACCAACTCACTTTC | |
| | | GGGAAGGGGACCAAACTCTCGGTCATACCAA | |
| | Variable-a | GQNIDQPTEMTATEGAIVQINCTYQTSGFNGLFWYQQHAGEAPTFLS | 618 |
| | | YNVLDGLEEKGRFSSFLSRSKGYSYLLLKELQMKDSASYLCAVRSPH | |
| | | GSGAGSYQLTFGKGTKLSVIP | |
| | CDR1-a | TSGFNG | 450 |
| | CDR2-a | NVLDGL | 451 |
| | CDR3-a | CAVRSPHGSGAGSYQLTF | 619 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GKGTKLSVIP | 489 |
| TCRB- | b-Variable | GATACTGGAGTCTCCCAGGACCCCAGACACAAGATCACAAAGAG | 620 |
| 168 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCGACGACGGTAGCTCCTACGAGCAGTACT | |
| | | TCGGGCCGGGCACCAGGCTCACGGTCACAG | |
| | Variable-b | DTGVSQDPRHKITKRGQNVTFRCDPISEHNRLYWYRQTLGQGPEFLT | 621 |
| | | YFQNEAQLEKSRLLSDRFSAERPKGSFSTLEIQRTEQGDSAMYLCASS | |
| | | DDGSSYEQYFGPGTRLTVT | |
| | CDR1-b | SEHNR | 442 |
| | CDR2-b | FQNEAQ | 443 |
| | CDR3-b | CASSDDGSSYEQYF | 622 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GPGTRLTVT | 85 |
| TCRA- | a-Variable | GACCAGCAAGTTAAGCAAAATTCACCATCCCTGAGCGTCCAGGA | 623 |
| 168 | nucleotide | AGGAAGAATTTCTATTCTGAACTGTGACTATACTAACAGCATGTT | |
| | | TGATTATTTCCTATGGTACAAAAAATACCCTGCTGAAGGTCCTAC | |
| | | ATTCCTGATATCTATAAGTTCCATTAAGGATAAAAATGAAGATGG | |
| | | AAGATTCACTGTTTTCTTAAACAAAAGTGCCAAGCACCTCTCTCT | |
| | | GCACATTGTGCCCTCCCAGCCTGGAGACTCTGCAGTGTACTTCTG | |
| | | TGCAGCAAGCGGAGGGGTAGGCACCAATGCAGGCAAATCAACCT | |
| | | TTGGGGATGGGACTACGCTCACTGTGAAGCCAA | |
| | Variable-a | DQQVKQNSPSLSVQEGRISILNCDYTNSMFDYFLWYKKYPAEGPTFL | 624 |
| | | ISISSIKDKNEDGRFTVFLNKSAKHLSLHIVPSQPGDSA VYFCAASGG | |
| | | VGTNAGKSTFGDGTTLTVKP | |
| | CDR1-a | NSMFDY | 303 |
| | CDR2-a | ISSIKDK | 304 |
| | CDR3-a | CAASGGVGTNAGKSTF | 625 |
| | FWR1-a | DQQVKQNSPSLSVQEGRISILNCDYT | 306 |
| | FWR2-a | FLWYKKYPAEGPTFLIS | 307 |
| | FWR3-a | NEDGRFTVFLNKSAKHLSLHIVPSQPGDSAVYF | 308 |
| | FWR4-a | GDGTTLTVKP | 626 |
| TCRB- | b-Variable | GGAGCTGGAGTCTCCCAGTCCCCCAGTAACAAGGTCACAGAGAA | 627 |
| 169 | nucleotide | GGGAAAGGATGTAGAGCTCAGGTGTGATCCAATTTCAGGTCATA | |
| | | CTGCCCTTTACTGGTACCGACAGAGCCTGGGGCAGGGCCTGGAGT | |
| | | TTTTAATTTACTTCCAAGGCAACAGTGCACCAGACAAATCAGGGC | |
| | | TGCCCAGTGATCGCTTCTCTGCAGAGAGGACTGGGGGATCCGTCT | |
| | | CCACTCTGACGATCCAGCGCACACAGCAGGAGGACTCGGCCGTG | |
| | | TATCTCTGTGCCAGCAGCTTCGGGGGGGACCCACGCACTCAGCCC | |
| | | CAGCATTTTGGTGATGGGACTCGACTCTCCATCCTAG | |
| | Variable-b | GAGVSQSPSNKVTEKGKDVELRCDPISGHTALYWYRQSLGQGLEFLI | 628 |
| | | YFQGNSAPDKSGLPSDRFSAERTGGSVSTLTIQRTQQEDSAVYLCAS | |
| | | SFGGDPRTQPQHFGDGTRLSIL | |
| | CDR1-b | SGHTA | 263 |
| | CDR2-b | FQGNSA | 629 |
| | CDR3-b | CASSFGGDPRTQPQHF | 630 |
| | FWR1-b | GAGVSQSPSNKVTEKGKDVELRCDPI | 631 |
| | FWR2-b | LYWYRQSLGQGLEFLIY | 632 |
| | FWR3-b | PDKSGLPSDRFSAERTGGSVSTLTIQRTQQEDSAVYL | 633 |
| | FWR4-b | GDGTRLSIL | 27 |
| TCRA- | a-Variable | CTTGCTAAGACCACCCAGCCCATCTCCATGGACTCATATGAAGGA | 634 |
| 169 | nucleotide | CAAGAAGTGAACATAACCTGTAGCCACAACAACATTGCTACAAA | |
| | | TGATTATATCACGTGGTACCAACAGTTTCCCAGCCAAGGACCACG | |
| | | ATTTATTATTCAAGGATACAAGACAAAAGTTACAAACGAAGTGG | |
| | | CCTCCCTGTTTATCCCTGCCGACAGAAAGTCCAGCACTCTGAGCC | |
| | | TGCCCCGGGTTTCCCTGAGCGACACTGCTGTGTACTACTGCCTCG | |
| | | TGGGTCTATCTAACTTTGGAAATGAGAAATTAACCTTTGGGACTG | |
| | | GAACAAGACTCACCATCATACCCA | |
| | Variable-a | LAKTTQPISMDSYEGQEVNITCSHNNIATNDYITWYQQFPSQGPRFII | 635 |
| | | QGYKTKVTNEVASLFIPADRKSSTLSLPRVSLSDTAVYYCLVGLSNF | |
| | | GNEKLTFGTGTRLTIIP | |
| | CDR1-a | NIATNDY | 594 |
| | CDR2-a | GYKTK | 595 |
| | CDR3-a | CLVGLSNFGNEKLTF | 636 |
| | FWR1-a | LAKTTQPISMDSYEGQEVNITCSHN | 597 |
| | FWR2-a | ITWYQQFPSQGPRFIIQ | 598 |
| | FWR3-a | VTNEVASLFIPADRKSSTLSLPRVSLSDTAVYY | 599 |
| | FWR4-a | GTGTRLTIIP | 292 |
| TCRB- | b-Variable | GACACTGAAGTTACCCAGACACCAAAACACCTGGTCATGGGAAT | 637 |
| 170 | nucleotide | GACAAATAAGAAGTCTTTGAAATGTGAACAACATATGGGGCACA | |
| | | GGGCTATGTATTGGTACAAGCAGAAAGCTAAGAAGCCACCGGAG | |
| | | CTCATGTTTGTCTACAGCTATGAGAAACTCTCTATAAATGAAAGT | |
| | | GTGCCAAGTCGCTTCTCACCTGAATGCCCCAACAGCTCTCTCTTA | |
| | | AACCTTCACCTACACGCCCTGCAGCCAGAAGACTCAGCCCTGTAT | |
| | | CTCTGCGCCAGCAGCCAAGGTCAGGGTCTGAACACTGAAGCTTTC | |
| | | TTTGGACAAGGCACCAGACTCACAGTTGTAG | |
| | Variable-b | DTEVTQTPKHLVMGMTNKKSLKCEQHMGHRAMYWYKQKAKKPP | 638 |
| | | ELMFVYSYEKLSINESVPSRFSPECPNSSLLNLHLHALQPEDSALYLC | |
| | | ASSQGQGLNTEAFFGQGTRLTVV | |
| | CDR1-b | MGHRA | 639 |
| | CDR2-b | YSYEKL | 640 |
| | CDR3-b | CASSQGQGLNTEAFF | 641 |
| | FWR1-b | DTEVTQTPKHLVMGMTNKKSLKCEQH | 642 |
| | FWR2-b | MYWYKQKAKKPPELMFV | 643 |
| | FWR3-b | SINESVPSRESPECPNSSLLNLHLHALQPEDSALYL | 644 |
| | FWR4-b | GQGTRLTVV | 246 |
| TCRA- | a-Variable | GGACAAAACATTGACCAGCCCACTGAGATGACAGCTACGGAAGG | 645 |
| 170 | nucleotide | TGCCATTGTCCAGATCAACTGCACGTACCAGACATCTGGGTTCAA | |
| | | CGGGCTGTTCTGGTACCAGCAACATGCTGGCGAAGCACCTACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTG | |
| | | AGAGCATACTCTGGGGCTGGGAGTTACCAACTCACTTTCGGGAAG | |
| | | GGGACCAAACTCTCGGTCATACCAA | |
| | Variable-a | GQNIDQPTEMTATEGAIVQINCTYQTSGFNGLFWYQQHAGEAPTFLS | 646 |
| | | YNVLDGLEEKGRFSSFLSRSKGYSYLLLKELQMKDSASYLCAVRAY | |
| | | SGAGSYQLTFGKGTKLSVIP | |
| | CDR1-a | TSGFNG | 450 |
| | CDR2-a | NVLDGL | 451 |
| | CDR3-a | CAVRAYSGAGSYQLTF | 647 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GKGTKLSVIP | 489 |
| TCRB- | b-Variable | GGTGCTGTCGTCTCTCAACATCCGAGCTGGGTTATCTGTAAGAGT | 648 |
| 171 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGAGATTGGGGCAGGGCCCCAGATA | |
| | | CGCAGTATTTTGGCCCAGGCACCCGGCTGACAGTGCTCG | |
| | Variable-b | GAVVSQHPSWVICKSGTSVKIECRSLDFQATTMFWYRQFPKQSLML | 649 |
| | | MATSNEGSKATYEQGVEKDKFLINHASLTLSTLTVTSAHPEDSSFYIC | |
| | | SARDWGRAPDTQYFGPGTRLTVL | |
| | CDR1-b | DFQATT | 79 |
| | CDR2-b | SNEGSKA | 80 |
| | CDR3-b | CSARDWGRAPDTQYF | 650 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVL | 9 |
| TCRA- | a-Variable | GGAGAGGATGTGGAGCAGAGTCTTTTCCTGAGTGTCCGAGAGGG | 651 |
| 171 | nucleotide | AGACAGCTCCGTTATAAACTGCACTTACACAGACAGCTCCTCCAC | |
| | | CTACTTATACTGGTATAAGCAAGAACCTGGAGCAGGTCTCCAGTT | |
| | | GCTGACGTATATTTTTTCAAATATGGACATGAAACAAGACCAAAG | |
| | | ACTCACTGTTCTATTGAATAAAAAGGATAAACATCTGTCTCTGCG | |
| | | CATTGCAGACACCCAGACTGGGGACTCAGCTATCTACTTCTGTGC | |
| | | AGAGATTAACACCGGTAACCAGTTCTATTTTGGGACAGGGACAA | |
| | | GTTTGACGGTCATTCCAA | |
| | Variable-a | GEDVEQSLFLSVREGDSSVINCTYTDSSSTYLYWYKQEPGAGLQLLT | 652 |
| | | YIFSNMDMKQDQRLTVLLNKKDKHLSLRIADTQTGDSAIYFCAEINT | |
| | | GNQFYFGTGTSLTVIP | |
| | CDR1-a | DSSSTY | 314 |
| | CDR2-a | IFSNMDM | 315 |
| | CDR3-a | CAEINTGNQFYF | 653 |
| | FWR1-a | GEDVEQSLFLSVREGDSSVINCTYT | 317 |
| | FWR2-a | LYWYKQEPGAGLQLLTY | 318 |
| | FWR3-a | KQDQRLTVLLNKKDKHLSLRIADTQTGDSAIYF | 319 |
| | FWR4-a | GTGTSLTVIP | 94 |
| TCRB- | b-Variable | GATTCTGGAGTCACACAAACCCCAAAGCACCTGATCACAGCAAC | 654 |
| 172 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCGCACAAGGGGACAGGGGGGATGGAGCTTTCT | |
| | | TTGGACAAGGCACCAGACTCACAGTTGTAG | |
| | Variable-b | DSGVTQTPKHLITATGQRVTLRCSPRSGDLSVYWYQQSLDQGLQFLI | 655 |
| | | QYYNGEERAKGNILERFSAQQFPDLHSELNLSSLELGDSALYFCASS | |
| | | AQGDRGDGAFFGQGTRLTVV | |
| | CDR1-b | SGDLS | 383 |
| | CDR2-b | YYNGEE | 384 |
| | CDR3-b | CASSAQGDRGDGAFF | 656 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GQGTRLTVV | 246 |
| TCRA- | a-Variable | GCCCAGAAGATAACTCAAACCCAACCAGGAATGTTCGTGCAGGA | 657 |
| 172 | nucleotide | AAAGGAGGCTGTGACTCTGGACTGCACATATGACACCAGTGATC | |
| | | CAAGTTATGGTCTATTCTGGTACAAGCAGCCCAGCAGTGGGGAA | |
| | | ATGATTTTTCTTATTTATCAGGGGTCTTATGACCAGCAAAATGCA | |
| | | ACAGAAGGTCGCTACTCATTGAATTTCCAGAAGGCAAGAAAATC | |
| | | CGCCAACCTTGTCATCTCCGCTTCACAACTGGGGGACTCAGCAAT | |
| | | GTATTTCTGTGCAATGAGAGAGACGGGTAGCAACTATAAACTGA | |
| | | CATTTGGAAAAGGAACTCTCTTAACCGTGAATCCAA | |
| | Variable-a | AQKITQTQPGMFVQEKEAVTLDCTYDTSDPSYGLFWYKQPSSGEMI | 658 |
| | | FLIYQGSYDQQNATEGRYSLNFQKARKSANLVISASQLGDSAMYFC | |
| | | AMRETGSNYKLTFGKGTLLTVNP | |
| | CDR1-a | TSDPSYG | 659 |
| | CDR2-a | QGSYDQQN | 660 |
| | CDR3-a | CAMRETGSNYKLTF | 661 |
| | FWR1-a | AQKITQTQPGMFVQEKEAVTLDCTYD | 406 |
| | FWR2-a | LFWYKQPSSGEMIFLIY | 407 |
| | FWR3-a | ATEGRYSLNFQKARKSANLVISASQLGDSAMYF | 408 |
| | FWR4-a | GKGTLLTVNP | 662 |
| TCRB- | b-Variable | GCTGCTGGAGTCATCCAGTCCCCAAGACATCTGATCAAAGAAAA | 663 |
| 173 | nucleotide | GAGGGAAACAGCCACTCTGAAATGCTATCCTATCCCTAGACACG | |
| | | ACACTGTCTACTGGTACCAGCAGGGTCCAGGTCAGGACCCCCAGT | |
| | | TCCTCATTTCGTTTTATGAAAAGATGCAGAGCGATAAAGGAAGCA | |
| | | TCCCTGATCGATTCTCAGCTCAACAGTTCAGTGACTATCATTCTGA | |
| | | ACTGAACATGAGCTCCTTGGAGCTGGGGGACTCAGCCCTGTACTT | |
| | | CTGTGCCAGCAGCTTAGCAGGGGAGGGTGAAGCTTTCTTTGGACA | |
| | | AGGCACCAGACTCACAGTTGTAG | |
| | Variable-b | AAGVIQSPRHLIKEKRETATLKCYPIPRHDTVYWYQQGPGQDPQFLI | 664 |
| | | SFYEKMQSDKGSIPDRFSAQQFSDYHSELNMSSLELGDSALYFCASS | |
| | | LAGEGEAFFGQGTRLTVV | |
| | CDR1-b | PRHDT | 665 |
| | CDR2-b | FYEKMQ | 666 |
| | CDR3-b | CASSLAGEGEAFF | 667 |
| | FWR1-b | AAGVIQSPRHLIKEKRETATLKCYPI | 668 |
| | FWR2-b | VYWYQQGPGQDPQFLIS | 669 |
| | FWR3-b | SDKGSIPDRFSAQQFSDYHSELNMSSLELGDSALYF | 670 |
| | FWR4-b | GQGTRLTVV | 246 |
| TCRA- | a-Variable | CAGAAGGAGGTGGAGCAGGATCCTGGACCACTCAGTGTTCCAGA | 671 |
| 173 | nucleotide | GGGAGCCATTGTTTCTCTCAACTGCACTTACAGCAACAGTGCTTT | |
| | | TCAATACTTCATGTGGTACAGACAGTATTCCAGAAAAGGCCCTGA | |
| | | GTTGCTGATGTACACATACTCCAGTGGTAACAAAGAAGATGGAA | |
| | | GGTTTACAGCACAGGTCGATAAATCCAGCAAGTATATCTCCTTGT | |
| | | TCATCAGAGACTCACAGCCCAGTGATTCAGCCACCTACCTCTGTG | |
| | | CAGTCGGGGGCGGATCTGAAAAGCTGGTCTTTGGAAAGGGAACG | |
| | | AAACTGACAGTAAACCCAT | |
| | Variable-a | QKEVEQDPGPLSVPEGAIVSLNCTYSNSAFQYFMWYRQYSRKGPEL | 672 |
| | | LMYTYSSGNKEDGRFTAQVDKSSKYISLFIRDSQPSDSATYLCAVGG | |
| | | GSEKLVFGKGTKLTVNP | |
| | CDR1-a | NSAFQY | 673 |
| | CDR2-a | TYSSGN | 674 |
| | CDR3-a | CAVGGGSEKLVF | 675 |
| | FWR1-a | QKEVEQDPGPLSVPEGAIVSLNCTYS | 676 |
| | FWR2-a | FMWYRQYSRKGPELLMY | 677 |
| | FWR3-a | KEDGRFTAQVDKSSKYISLFIRDSQPSDSATYL | 678 |
| | FWR4-a | GKGTKLTVNP | 339 |
| TCRB- | b-Variable | GATGTGAAAGTAACCCAGAGCTCGAGATATCTAGTCAAAAGGAC | 679 |
| 174 | nucleotide | GGGAGAGAAAGTTTTTCTGGAATGTGTCCAGGATATGGACCATG | |
| | | AAAATATGTTCTGGTATCGACAAGACCCAGGTCTGGGGCTACGGC | |
| | | TGATCTATTTCTCATATGATGTTAAAATGAAAGAAAAAGGAGATA | |
| | | TTCCTGAGGGGTACAGTGTCTCTAGAGAGAAGAAGGAGCGCTTCT | |
| | | CCCTGATTCTGGAGTCCGCCAGCACCAACCAGACATCTATGTACC | |
| | | TCTGTGCCAGCAGTTGGACAGAAGAGACCTACGAGCAGTACTTC | |
| | | GGGCCGGGCACCAGGCTCACGGTCACAG | |
| | Variable-b | DVKVTQSSRYLVKRTGEKVFLECVQDMDHENMFWYRQDPGLGLRL | 680 |
| | | IYFSYDVKMKEKGDIPEGYSVSREKKERFSLILESASTNQTSMYLCAS | |
| | | SWTEETYEQYFGPGTRLTVT | |
| | CDR1-b | MDHEN | 199 |
| | CDR2-b | SYDVKM | 200 |
| | CDR3-b | CASSWTEETYEQYF | 681 |
| | FWR1-b | DVKVTQSSRYLVKRTGEKVFLECVQD | 202 |
| | FWR2-b | MFWYRQDPGLGLRLIYF | 203 |
| | FWR3-b | KEKGDIPEGYSVSREKKERFSLILESASTNQTSMYL | 204 |
| | FWR4-b | GPGTRLTVT | 85 |
| TCRA- | a-Variable | GCTCAGTCAGTGGCTCAGCCGGAAGATCAGGTCAACGTTGCTGA | 682 |
| 174 | nucleotide | AGGGAATCCTCTGACTGTGAAATGCACCTATTCAGTCTCTGGAAA | |
| | | CCCTTATCTTTTTTGGTATGTTCAATACCCCAACCGAGGCCTCCAG | |
| | | TTCCTTCTGAAATACATCACAGGGGATAACCTGGTTAAAGGCAGC | |
| | | TATGGCTTTGAAGCTGAATTTAACAAGAGCCAAACCTCCTTCCAC | |
| | | CTGAAGAAACCATCTGCCCTTGTGAGCGACTCCGCTTTGTACTTC | |
| | | TGTGCTGTGAGAGCGTACAATAACAATGACATGCGCTTTGGAGCA | |
| | | GGGACCAGACTGACAGTAAAACCAA | |
| | Variable-a | AQSVAQPEDQVNVAEGNPLTVKCTYSVSGNPYLFWYVQYPNRGLQ | 683 |
| | | FLLKYITGDNLVKGSYGFEAEFNKSQTSFHLKKPSALVSDSALYFCA | |
| | | VRAYNNNDMRFGAGTRLTVKP | |
| | CDR1-a | VSGNPY | 427 |
| | CDR2-a | YITGDNLV | 428 |
| | CDR3-a | CAVRAYNNNDMRF | 684 |
| | FWR1-a | AQSVAQPEDQVNVAEGNPLTVKCTYS | 430 |
| | FWR2-a | LFWYVQYPNRGLQFLLK | 431 |
| | FWR3-a | KGSYGFEAEFNKSQTSFHLKKPSALVSDSALYF | 432 |
| | FWR4-a | GAGTRLTVKP | 685 |
| TCRB- | b-Variable | GATGCTGGAGTTATCCAGTCACCCCGGCACGAGGTGACAGAGAT | 686 |
| 175 | nucleotide | GGGACAAGAAGTGACTCTGAGATGTAAACCAATTTCAGGACACG | |
| | | ACTACCTTTTCTGGTACAGACAGACCATGATGCGGGGACTGGAGT | |
| | | TGCTCATTTACTTTAACAACAACGTTCCGATAGATGATTCAGGGA | |
| | | TGCCCGAGGATCGATTCTCAGCTAAGATGCCTAATGCATCATTCT | |
| | | CCACTCTGAAGATCCAGCCCTCAGAACCCAGGGACTCAGCTGTGT | |
| | | ACTTCTGTGCCAGCAGTTTAGGGACAGGTAGTGAGCAGTTCTTCG | |
| | | GGCCAGGGACACGGCTCACCGTGCTAG | |
| | Variable-b | DAGVIQSPRHEVTEMGQEVTLRCKPISGHDYLFWYRQTMMRGLELL | 687 |
| | | IYFNNNVPIDDSGMPEDRFSAKMPNASFSTLKIQPSEPRDSAVYFCAS | |
| | | SLGTGSEQFFGPGTRLTVL | |
| | CDR1-b | SGHDY | 468 |
| | CDR2-b | FNNNVP | 469 |
| | CDR3-b | CASSLGTGSEQFF | 688 |
| | FWR1-b | DAGVIQSPRHEVTEMGQEVTLRCKPI | 471 |
| | FWR2-b | LFWYRQTMMRGLELLIY | 472 |
| | FWR3-b | IDDSGMPEDRFSAKMPNASFSTLKIQPSEPRDSAVYF | 473 |
| | FWR4-b | GPGTRLTVL | 9 |
| TCRA- | a-Variable | AAACAGGAGGTGACACAGATTCCTGCAGCTCTGAGTGTCCCAGA | 689 |
| 175 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTACGTACCATTTCAGATGGCCAGAAGCTGCTCTTTGCAAG | |
| | | GGGGACCATGTTAAAGGTGGATCTTA | |
| | Variable-a | KQEVTQIPAALSVPEGENLVLNCSFTDSAIYNLQWFRQDPGKGLTSL | 690 |
| | | LLIQSSQREQTSGRLNASLDKSSGRSTLYIAASQPGDSATYLCAVRTI | |
| | | SDGQKLLFARGTMLKVDL | |
| | CDR1-a | DSAIYN | 118 |
| | CDR2-a | IQSSQRE | 119 |
| | CDR3-a | CAVRTISDGQKLLF | 691 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | ARGTMLKVDL | 392 |
| TCRB- | b-Variable | GCTGCTGGAGTCATCCAGTCCCCAAGACATCTGATCAAAGAAAA | 692 |
| 176 | nucleotide | GAGGGAAACAGCCACTCTGAAATGCTATCCTATCCCTAGACACG | |
| | | ACACTGTCTACTGGTACCAGCAGGGTCCAGGTCAGGACCCCCAGT | |
| | | TCCTCATTTCGTTTTATGAAAAGATGCAGAGCGATAAAGGAAGCA | |
| | | TCCCTGATCGATTCTCAGCTCAACAGTTCAGTGACTATCATTCTGA | |
| | | ACTGAACATGAGCTCCTTGGAGCTGGGGGACTCAGCCCTGTACTT | |
| | | CTGTGCCAGCCGGATACGGGGGGGAATGAACACTGAAGCTTTCTT | |
| | | TGGACAAGGCACCAGACTCACAGTTGTAG | |
| | Variable-b | AAGVIQSPRHLIKEKRETATLKCYPIPRHDTVYWYQQGPGQDPQFLI | 693 |
| | | SFYEKMQSDKGSIPDRFSAQQFSDYHSELNMSSLELGDSALYFCASRI | |
| | | RGGMNTEAFFGQGTRLTVV | |
| | CDR1-b | PRHDT | 665 |
| | CDR2-b | FYEKMQ | 666 |
| | CDR3-b | CASRIRGGMNTEAFF | 694 |
| | FWR1-b | AAGVIQSPRHLIKEKRETATLKCYPI | 668 |
| | FWR2-b | VYWYQQGPGQDPQFLIS | 669 |
| | FWR3-b | SDKGSIPDRFSAQQFSDYHSELNMSSLELGDSALYF | 670 |
| | FWR4-b | GQGTRLTVV | 246 |
| TCRA- | a-Variable | GCTCAGTCAGTGGCTCAGCCGGAAGATCAGGTCAACGTTGCTGA | 695 |
| 176 | nucleotide | AGGGAATCCTCTGACTGTGAAATGCACCTATTCAGTCTCTGGAAA | |
| | | CCCTTATCTTTTTTGGTATGTTCAATACCCCAACCGAGGCCTCCAG | |
| | | TTCCTTCTGAAATACATCACAGGGGATAACCTGGTTAAAGGCAGC | |
| | | TATGGCTTTGAAGCTGAATTTAACAAGAGCCAAACCTCCTTCCAC | |
| | | CTGAAGAAACCATCTGCCCTTGTGAGCGACTCCGCTTTGTACTTC | |
| | | TGTGCTGTGAGAGCTCCTCTTTCAGATGGCCAGAAGCTGCTCTTT | |
| | | GCAAGGGGAACCATGTTAAAGGTGGATCTTA | |
| | Variable-a | AQSVAQPEDQVNVAEGNPLTVKCTYSVSGNPYLFWYVQYPNRGLQ | 696 |
| | | FLLKYITGDNLVKGSYGFEAEFNKSQTSFHLKKPSALVSDSALYFCA | |
| | | VRAPLSDGQKLLFARGTMLKVDL | |
| | CDR1-a | VSGNPY | 427 |
| | CDR2-a | YITGDNLV | 428 |
| | CDR3-a | CAVRAPLSDGQKLLF | 697 |
| | FWR1-a | AQSVAQPEDQVNVAEGNPLTVKCTYS | 430 |
| | FWR2-a | LFWYVQYPNRGLQFLLK | 431 |
| | FWR3-a | KGSYGFEAEFNKSQTSFHLKKPSALVSDSALYF | 432 |
| | FWR4-a | ARGTMLKVDL | 392 |
| TCRB- | b-Variable | GGTGCTGTCGTCTCTCAACATCCGAGCTGGGTTATCTGTAAGAGT | 698 |
| 177 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGAGACAGGACTAGCGGGGGGAGC | |
| | | TCCTACGAGCAGTACTTCGGGCCGGGCACCAGGCTCACGGTCACA | |
| | | G | |
| | Variable-b | GAVVSQHPSWVICKSGTSVKIECRSLDFQATTMFWYRQFPKQSLML | 699 |
| | | MATSNEGSKATYEQGVEKDKFLINHASLTLSTLTVTSAHPEDSSFYIC | |
| | | SARDRTSGGSSYEQYFGPGTRLTVT | |
| | CDR1-b | DFQATT | 79 |
| | CDR2-b | SNEGSKA | 80 |
| | CDR3-b | CSARDRTSGGSSYEQYF | 700 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVT | 85 |
| TCRA- | a-Variable | CTTGCTAAGACCACCCAGCCCATCTCCATGGACTCATATGAAGGA | 701 |
| 177 | nucleotide | CAAGAAGTGAACATAACCTGTAGCCACAACAACATTGCTACAAA | |
| | | TGATTATATCACGTGGTACCAACAGTTTCCCAGCCAAGGACCACG | |
| | | ATTTATTATTCAAGGATACAAGACAAAAGTTACAAACGAAGTGG | |
| | | CCTCCCTGTTTATCCCTGCCGACAGAAAGTCCAGCACTCTGAGCC | |
| | | TGCCCCGGGTTTCCCTGAGCGACACTGCTGTGTACTACTGCCTCG | |
| | | TGGGTTCCCTCAACAAACTGGTCTTTGGCGCAGGAACCATTCTGA | |
| | | GAGTCAAGTCCT | |
| | Variable-a | LAKTTQPISMDSYEGQEVNITCSHNNIATNDYITWYQQFPSQGPRFII | 702 |
| | | QGYKTKVTNEVASLFIPADRKSSTLSLPRVSLSDTAVYYCLVGSLNK | |
| | | LVFGAGTILRVKS | |
| | CDR1-a | NIATNDY | 594 |
| | CDR2-a | GYKTK | 595 |
| | CDR3-a | CLVGSLNKLVF | 703 |
| | FWR1-a | LAKTTQPISMDSYEGQEVNITCSHN | 597 |
| | FWR2-a | ITWYQQFPSQGPRFIIQ | 598 |
| | FWR3-a | VTNEVASLFIPADRKSSTLSLPRVSLSDTAVYY | 599 |
| | FWR4-a | GAGTILRVKS | 18 |
| TCRB- | b-Variable | GAAACGGGAGTTACGCAGACACCAAGACACCTGGTCATGGGAAT | 704 |
| 178 | nucleotide | GACAAATAAGAAGTCTTTGAAATGTGAACAACATCTGGGTCATA | |
| | | ACGCTATGTATTGGTACAAGCAAAGTGCTAAGAAGCCACTGGAG | |
| | | CTCATGTTTGTCTACAGTCTTGAAGAACGGGTTGAAAACAACAGT | |
| | | GTGCCAAGTCGCTTCTCACCTGAATGCCCCAACAGCTCTCACTTA | |
| | | TTCCTTCACCTACACACCCTGCAGCCAGAAGACTCGGCCCTGTAT | |
| | | CTCTGCGCCAGCAGCCAATGGGGGGGCGGGAGCTCCTACAATGA | |
| | | GCAGTTCTTCGGGCCAGGGACACGGCTCACCGTGCTAG | |
| | Variable-b | ETGVTQTPRHLVMGMTNKKSLKCEQHLGHNAMYWYKQSAKKPLE | 705 |
| | | LMFVYSLEERVENNSVPSRFSPECPNSSHLFLHLHTLQPEDSALYLCA | |
| | | SSQWGGGSSYNEQFFGPGTRLTVL | |
| | CDR1-b | LGHNA | 169 |
| | CDR2-b | YSLEER | 706 |
| | CDR3-b | CASSQWGGGSSYNEQFF | 707 |
| | FWR1-b | ETGVTQTPRHLVMGMTNKKSLKCEQH | 172 |
| | FWR2-b | MYWYKQSAKKPLELMFV | 173 |
| | FWR3-b | VENNSVPSRFSPECPNSSHLFLHLHTLQPEDSALYL | 708 |
| | FWR4-b | GPGTRLTVL | 9 |
| TCRA- | a-Variable | GGAATACAAGTGGAGCAGAGTCCTCCAGACCTGATTCTCCAGGA | 709 |
| 178 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTGATT | |
| | | ACCTCAGGAACCTACAAATACATCTTTGGAACAGGCACCAGGCT | |
| | | GAAGGTTTTAGCAA | |
| | Variable-a | GIQVEQSPPDLILQEGANSTLRCNFSDSVNNLQWFHQNPWGQLINLF | 710 |
| | | YIPSGTKQNGRLSATTVATERYSLLYISSSQTTDSGVYFCAVITSGTY | |
| | | KYIFGTGTRLKVLA | |
| | CDR1-a | DSVNN | 506 |
| | CDR2-a | IPSGT | 507 |
| | CDR3-a | CAVITSGTYKYIF | 711 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GTGTRLKVLA | 587 |
| TCRB- | b-Variable | AATGCTGGTGTCACTCAGACCCCAAAATTCCAGGTCCTGAAGACA | 712 |
| 179 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTACGACCGGGACACTCTACGAGCAGTACTTCGG | |
| | | GCCGGGCACCAGGCTCACGGTCACAG | |
| | Variable-b | NAGVTQTPKFQVLKTGQSMTLQCAQDMNHEYMSWYRQDPGMGLR | 713 |
| | | LIHYSVGAGITDQGEVPNGYNVSRSTTEDFPLRLLSAAPSQTSVYFCA | |
| | | SSTTGTLYEQYFGPGTRLTVT | |
| | CDR1-b | MNHEY | 252 |
| | CDR2-b | SVGAGI | 253 |
| | CDR3-b | CASSTTGTLYEQYF | 714 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GPGTRLTVT | 85 |
| TCRA- | a-Variable | GGAGAGAATGTGGAGCAGCATCCTTCAACCCTGAGTGTCCAGGA | 715 |
| 179 | nucleotide | GGGAGACAGCGCTGTTATCAAGTGTACTTATTCAGACAGTGCCTC | |
| | | AAACTACTTCCCTTGGTATAAGCAAGAACTTGGAAAAAGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCAACTGGGGGGTATTCAGGAGGAGGTGCTGACGGACTC | |
| | | ACCTTTGGCAAAGGGACTCATCTAATCATCCAGCCCT | |
| | Variable-a | GENVEQHPSTLSVQEGDSAVIKCTYSDSASNYFPWYKQELGKRPQLI | 716 |
| | | IDIRSNVGEKKDQRIA VTLNKTAKHFSLHITETQPEDSAVYFCAATGG | |
| | | YSGGGADGLTFGKGTHLIIQP | |
| | CDR1-a | DSASNY | 207 |
| | CDR2-a | IRSNVGE | 208 |
| | CDR3-a | CAATGGYSGGGADGLTF | 717 |
| | FWR1-a | GENVEQHPSTLSVQEGDSAVIKCTYS | 210 |
| | FWR2-a | FPWYKQELGKRPQLIID | 211 |
| | FWR3-a | KKDQRIAVTLNKTAKHFSLHITETQPEDSAVYF | 212 |
| | FWR4-a | GKGTHLIIQP | 363 |
| TCRB- | b-Variable | GATGGTGGAATCACTCAGTCCCCAAAGTACCTGTTCAGAAAGGA | 775 |
| 180 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGTATTGCAGGGGCGGACTACGAGCAGTACTT | |
| | | CGGGCCGGGCACCAGGCTCACGGTCACAG | |
| | Variable-b | DGGITQSPKYLFRKEGQNVTLSCEQNLNHDAMYWYRQDPGQGLRLI | 776 |
| | | YYSQIVNDFQKGDIAEGYSVSREKKESFPLTVTSAQKNPTAFYLCAS | |
| | | SIAGADYEQYFGPGTRLTVT | |
| | CDR1-b | LNHDA | 216 |
| | CDR2-b | SQIVND | 217 |
| | CDR3-b | CASSIAGADYEQYF | 777 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVT | 85 |
| TCRA- | a-Variable | GGAGAGAATGTGGAGCAGCATCCTTCAACCCTGAGTGTCCAGGA | 778 |
| 180 | nucleotide | GGGAGACAGCGCTGTTATCAAGTGTACTTATTCAGACAGTGCCTC | |
| | | AAACTACTTCCCTTGGTATAAGCAAGAACTTGGAAAAAGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCACAGCCACCAACGCCAACGAACACCGGTAACCAGTTC | |
| | | TATTTTGGGACAGGGACAAGTTTGACGGTCATTCCAA | |
| | Variable-a | GENVEQHPSTLSVQEGDSAVIKCTYSDSASNYFPWYKQELGKRPQLI | 779 |
| | | IDIRSNVGEKKDQRIA VTLNKTAKHFSLHITETQPEDSAVYFCAAQPP | |
| | | TPTNTGNQFYFGTGTSLTVIP | |
| | CDR1-a | DSASNY | 207 |
| | CDR2-a | IRSNVGE | 208 |
| | CDR3-a | CAAQPPTPTNTGNQFYF | 780 |
| | FWR1-a | GENVEQHPSTLSVQEGDSAVIKCTYS | 210 |
| | FWR2-a | FPWYKQELGKRPQLIID | 211 |
| | FWR3-a | KKDQRIAVTLNKTAKHFSLHITETQPEDSAVYF | 212 |
| | FWR4-a | GTGTSLTVIP | 94 |
| TCRB- | b-Variable | GCTGCTGGAGTCATCCAGTCCCCAAGACATCTGATCAAAGAAAA | 781 |
| 181 | nucleotide | GAGGGAAACAGCCACTCTGAAATGCTATCCTATCCCTAGACACG | |
| | | ACACTGTCTACTGGTACCAGCAGGGTCCAGGTCAGGACCCCCAGT | |
| | | TCCTCATTTCGTTTTATGAAAAGATGCAGAGCGATAAAGGAAGCA | |
| | | TCCCTGATCGATTCTCAGCTCAACAGTTCAGTGACTATCATTCTGA | |
| | | ACTGAACATGAGCTCCTTGGAGCTGGGGGACTCAGCCCTGTACTT | |
| | | CTGTGCCAGCAGACCACAGGGAACTAATGAAAAACTGTTTTTTGG | |
| | | CAGTGGAACCCAGCTCTCTGTCTTGG | |
| | Variable-b | AAGVIQSPRHLIKEKRETATLKCYPIPRHDTVYWYQQGPGQDPQFLI | 782 |
| | | SFYEKMQSDKGSIPDRFSAQQFSDYHSELNMSSLELGDSALYFCASR | |
| | | PQGTNEKLFFGSGTQLSVL | |
| | CDR1-b | PRHDT | 665 |
| | CDR2-b | FYEKMQ | 666 |
| | CDR3-b | CASRPQGTNEKLFF | 783 |
| | FWR1-b | AAGVIQSPRHLIKEKRETATLKCYPI | 668 |
| | FWR2-b | VYWYQQGPGQDPQFLIS | 669 |
| | FWR3-b | SDKGSIPDRFSAQQFSDYHSELNMSSLELGDSALYF | 670 |
| | FWR4-b | GSGTQLSVL | 45 |
| TCRA- | a-Variable | AAACAGGAGGTGACGCAGATTCCTGCAGCTCTGAGTGTCCCAGA | 784 |
| 181 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGTGGAAACCAGTGGCTCTAGGTTGACCTTTGGGGAAGGAAC | |
| | | ACAGCTCACAGTGAATCCTG | |
| | Variable-a | KQEVTQIPAALSVPEGENLVLNCSFTDSAIYNLQWFRQDPGKGLTSL | 785 |
| | | LLIQSSQREQTSGRLNASLDKSSGRSTLYIAASQPGDSATYLCAVETS | |
| | | GSRLTFGEGTQLTVNP | |
| | CDR1-a | DSAIYN | 118 |
| | CDR2-a | IQSSQRE | 119 |
| | CDR3-a | CAVETSGSRLTF | 786 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GEGTQLTVNP | 787 |
| TCRB- | b-Variable | GGTGCTGGAGTCTCCCAGTCCCCTAGGTACAAAGTCGCAAAGAG | 788 |
| 182 | nucleotide | AGGACAGGATGTAGCTCTCAGGTGTGATCCAATTTCGGGTCATGT | |
| | | ATCCCTTTTTTGGTACCAACAGGCCCTGGGGCAGGGGCCAGAGTT | |
| | | TCTGACTTATTTCCAGAATGAAGCTCAACTAGACAAATCGGGGCT | |
| | | GCCCAGTGATCGCTTCTTTGCAGAAAGGCCTGAGGGATCCGTCTC | |
| | | CACTCTGAAGATCCAGCGCACACAGCAGGAGGACTCCGCCGTGT | |
| | | ATCTCTGTGCCAGCGGAGCTAGCGGGGGGGCTCACACCGGGGAG | |
| | | CTGTTTTTTGGAGAAGGCTCTAGGCTGACCGTACTGG | |
| | Variable-b | GAGVSQSPRYKVAKRGQDVALRCDPISGHVSLFWYQQALGQGPEFL | 789 |
| | | TYFQNEAQLDKSGLPSDRFFAERPEGSVSTLKIQRTQQEDSAVYLCA | |
| | | SGASGGAHTGELFFGEGSRLTVL | |
| | CDR1-b | SGHVS | 275 |
| | CDR2-b | FQNEAQ | 443 |
| | CDR3-b | CASGASGGAHTGELFF | 790 |
| | FWR1-b | GAGVSQSPRYKVAKRGQDVALRCDPI | 791 |
| | FWR2-b | LFWYQQALGQGPEFLTY | 792 |
| | FWR3-b | LDKSGLPSDRFFAERPEGSVSTLKIQRTQQEDSAVYL | 793 |
| | FWR4-b | GEGSRLTVL | 63 |
| TCRA- | a-Variable | GGAGAGGATGTGGAGCAGAGTCTTTTCCTGAGTGTCCGAGAGGG | 794 |
| 182 | nucleotide | AGACAGCTCCGTTATAAACTGCACTTACACAGACAGCTCCTCCAC | |
| | | CTACTTATACTGGTATAAGCAAGAACCTGGAGCAGGTCTCCAGTT | |
| | | GCTGACGTATATTTTTTCAAATATGGACATGAAACAAGACCAAAG | |
| | | ACTCACTGTTCTATTGAATAAAAAGGATAAACATCTGTCTCTGCG | |
| | | CATTGCAGACACCCAGACTGGGGACTCAGCTATCTACTTCTGTGC | |
| | | AGAGAGCGAGGGAGGAAAGCTTATCTTCGGACAGGGAACGGAGT | |
| | | TATCTGTGAAACCCA | |
| | Variable-a | GEDVEQSLFLSVREGDSSVINCTYTDSSSTYLYWYKQEPGAGLQLLT | 795 |
| | | YIFSNMDMKQDQRLTVLLNKKDKHLSLRIADTQTGDSAIYFCAESEG | |
| | | GKLIFGQGTELSVKP | |
| | CDR1-a | DSSSTY | 314 |
| | CDR2-a | IFSNMDM | 315 |
| | CDR3-a | CAESEGGKLIF | 793 |
| | FWR1-a | GEDVEQSLFLSVREGDSSVINCTYT | 317 |
| | FWR2-a | LYWYKQEPGAGLQLLTY | 318 |
| | FWR3-a | KQDQRLTVLLNKKDKHLSLRIADTQTGDSAIYF | 319 |
| | FWR4-a | GQGTELSVKP | 409 |
| TCRB- | b-Variable | GATGTGAAAGTAACCCAGAGCTCGAGATATCTAGTCAAAAGGAC | 797 |
| 183 | nucleotide | GGGAGAGAAAGTTTTTCTGGAATGTGTCCAGGATATGGACCATG | |
| | | AAAATATGTTCTGGTATCGACAAGACCCAGGTCTGGGGCTACGGC | |
| | | TGATCTATTTCTCATATGATGTTAAAATGAAAGAAAAAGGAGATA | |
| | | TTCCTGAGGGGTACAGTGTCTCTAGAGAGAAGAAGGAGCGCTTCT | |
| | | CCCTGATTCTGGAGTCCGCCAGCACCAACCAGACATCTATGTACC | |
| | | TCTGTGCCAGCAGACCTTTGAGGAGCAATCAGCCCCAGCATTTTG | |
| | | GTGATGGGACTCGACTCTCCATCCTAG | |
| | Variable-b | DVKVTQSSRYLVKRTGEKVFLECVQDMDHENMFWYRQDPGLGLRL | 798 |
| | | IYFSYDVKMKEKGDIPEGYSVSREKKERFSLILESASTNQTSMYLCAS | |
| | | RPLRSNQPQHFGDGTRLSIL | |
| | CDR1-b | MDHEN | 199 |
| | CDR2-b | SYDVKM | 200 |
| | CDR3-b | CASRPLRSNQPQHF | 799 |
| | FWR1-b | DVKVTQSSRYLVKRTGEKVFLECVQD | 202 |
| | FWR2-b | MFWYRQDPGLGLRLIYF | 203 |
| | FWR3-b | KEKGDIPEGYSVSREKKERFSLILESASTNQTSMYL | 204 |
| | FWR4-b | GDGTRLSIL | 27 |
| TCRA- | a-Variable | GGACAAAACATTGACCAGCCCACTGAGATGACAGCTACGGAAGG | 800 |
| 183 | nucleotide | TGCCATTGTCCAGATCAACTGCACGTACCAGACATCTGGGTTCAA | |
| | | CGGGCTGTTCTGGTACCAGCAACATGCTGGCGAAGCACCCACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTG | |
| | | AGAGAAGCTAATAATGCAGGCAACATGCTCACCTTTGGAGGGGG | |
| | | AACAAGGTTAATGGTCAAACCCC | |
| | Variable-a | GQNIDQPTEMTATEGAIVQINCTYQTSGFNGLFWYQQHAGEAPTFLS | 801 |
| | | YNVLDGLEEKGRFSSFLSRSKGYSYLLLKELQMKDSASYLCAVREA | |
| | | NNAGNMLTFGGGTRLMVKP | |
| | CDR1-a | TSGFNG | 450 |
| | CDR2-a | NVLDGL | 451 |
| | CDR3-a | CAVREANNAGNMLTF | 802 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GGGTRLMVKP | 141 |
| TCRB- | b-Variable | AGTGCTGTCATCTCTCAAAAGCCAAGCAGGGATATCTGTCAACGT | 803 |
| 184 | nucleotide | GGAACCTCCCTGACGATCCAGTGTCAAGTCGATAGCCAAGTCACC | |
| | | ATGATGTTCTGGTACCGTCAGCAACCTGGACAGAGCCTGACACTG | |
| | | ATCGCAACTGCAAATCAGGGCTCTGAGGCCACATATGAGAGTGG | |
| | | ATTTGTCATTGACAAGTTTCCCATCAGCCGCCCAAACCTAACATT | |
| | | CTCAACTCTGACTGTGAGCAACATGAGCCCTGAAGACAGCAGCA | |
| | | TATATCTCTGCAGCGTTGAGTATGAGCAGTTCTTCGGGCCAGGGA | |
| | | CACGGCTCACCGTGCTAG | |
| | Variable-b | SAVISQKPSRDICQRGTSLTIQCQVDSQVTMMFWYRQQPGQSLTLIA | 804 |
| | | TANQGSEATYESGFVIDKFPISRPNLTFSTLTVSNMSPEDSSIYLCSVE | |
| | | YEQFFGPGTRLTVL | |
| | CDR1-b | SQVTM | 97 |
| | CDR2-b | ANQGSEA | 98 |
| | CDR3-b | CSVEYEQFF | 805 |
| | FWR1-b | SAVISQKPSRDICQRGTSLTIQCQVD | 100 |
| | FWR2-b | MFWYRQQPGQSLTLIAT | 101 |
| | FWR3-b | TYESGFVIDKFPISRPNLTFSTLTVSNMSPEDSSIYL | 102 |
| | FWR4-b | GPGTRLTVL | 9 |
| TCRA- | a-Variable | CAGAAGGAGGTGGAGCAGAATTCTGGACCCCTCAGTGTTCCAGA | 806 |
| 184.1 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTTCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAACTCGATTGAAAACTTTGGAAATGAGAAATTAACCTTTG | |
| | | GGACTGGAACAAGACTCACCATCATACCCA | |
| | Variable-a | QKEVEQNSGPLSVPEGAIASLNCTYSDRGSQSFFWYRQYSGKSPELI | 807 |
| | | MFIYSNGDKEDGRFTAQLNKASQYVSLLIRDSQPSDSATYLCAVNSI | |
| | | ENFGNEKLTFGTGTRLTIIP | |
| | CDR1-a | DRGSQS | 12 |
| | CDR2-a | IYSNGD | 13 |
| | CDR3-a | CAVNSIENFGNEKLTF | 808 |
| | FWR1-a | QKEVEQNSGPLSVPEGAIASLNCTYS | 15 |
| | FWR2-a | FFWYRQYSGKSPELIMF | 16 |
| | FWR3-a | KEDGRFTAQLNKASQYVSLLIRDSQPSDSATYL | 17 |
| | FWR4-a | GTGTRLTIIP | 292 |
| TCRA- | a-Variable | GCTCAGACAGTCACTCAGTCTCAACCAGAGATGTCTGTGCAGGAG | 809 |
| 184.2 | nucleotide | GCAGAGACCGTGACCCTGAGCTGCACATATGACACCAGTGAGAG | |
| | | TGATTATTATTTATTCTGGTACAAGCAGCCTCCCAGCAGGCAGAT | |
| | | GATTCTCGTTATTCGCCAAGAAGCTTATAAGCAACAGAATGCAAC | |
| | | AGAGAATCGTTTCTCTGTGAACTTCCAGAAAGCAGCCAAATCCTT | |
| | | CAGTCTCAAGATCTCAGACTCACAGCTGGGGGATGCCGCGATGTA | |
| | | TTTCTGTGCTTATAGGATCTCCTGGTCAAATTCCGGGTATGCACTC | |
| | | AACTTCGGCAAAGGCACCTCGCTGTTGGTCACACCCC | |
| | Variable-a | AQTVTQSQPEMSVQEAETVTLSCTYDTSESDYYLFWYKQPPSRQMI | 810 |
| | | LVIRQEAYKQQNATENRFSVNFQKAAKSFSLKISDSQLGDAAMYFC | |
| | | AYRISWSNSGYALNFGKGTSLL VTP | |
| | CDR1-a | TSESDYY | 286 |
| | CDR2-a | QEAYKQQN | 287 |
| | CDR3-a | CAYRISWSNSGYALNF | 811 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDAAMYF | 291 |
| | FWR4-a | GKGTSLLVTP | 812 |
| TCRB- | b-Variable | AATGCCGGCGTCATGCAGAACCCAAGACACCTGGTCAGGAGGAG | 813 |
| 185 | nucleotide | GGGACAGGAGGCAAGACTGAGATGCAGCCCAATGAAAGGACAC | |
| | | AGTCATGTTTACTGGTATCGGCAGCTCCCAGAGGAAGGTCTGAAA | |
| | | TTCATGGTTTATCTCCAGAAAGAAAATATCATAGATGAGTCAGGA | |
| | | ATGCCAAAGGAACGATTTTCTGCTGAATTTCCCAAAGAGGGCCCC | |
| | | AGCATCCTGAGGATCCAGCAGGTAGTGCGAGGAGATTCGGCAGC | |
| | | TTATTTCTGTGCCAGCTCACAAGACAGGGCCGGGGCCAACGTCCT | |
| | | GACTTTCGGGGCCGGCAGCAGGCTGACCGTGCTGG | |
| | Variable-b | NAGVMQNPRHLVRRRGQEARLRCSPMKGHSHVYWYRQLPEEGLKF | 814 |
| | | MVYLQKENIIDESGMPKERFSAEFPKEGPSILRIQQVVRGDSAAYFCA | |
| | | SSQDRAGANVLTFGAGSRLTVL | |
| | CDR1-b | KGHSH | 144 |
| | CDR2-b | LQKENI | 145 |
| | CDR3-b | CASSQDRAGANVLTF | 815 |
| | FWR1-b | NAGVMQNPRHLVRRRGQEARLRCSPM | 147 |
| | FWR2-b | VYWYRQLPEEGLKFMVY | 148 |
| | FWR3-b | IDESGMPKERFSAEFPKEGPSILRIQQVVRGDSAAYF | 149 |
| | FWR4-b | GAGSRLTVL | 816 |
| TCRA- | a-Variable | GATGCTAAGACCACCCAGCCCCCCTCCATGGATTGCGCTGAAGGA | 817 |
| 185 | nucleotide | AGAGCTGCAAACCTGCCTTGTAATCACTCTACCATCAGTGGAAAT | |
| | | GAGTATGTGTATTGGTATCGACAGATTCACTCCCAGGGGCCACAG | |
| | | TATATCATTCATGGTCTAAAAAACAATGAAACCAATGAAATGGCC | |
| | | TCTCTGATCATCACAGAAGACAGAAAGTCCAGCACCTTGATCCTG | |
| | | CCCCACGCTACGCTGAGAGACACTGCTGTGTACTATTGCATCGTC | |
| | | CGAGGAGGTGCTGACGGACTCACCTTTGGCAAAGGGACTCATCT | |
| | | AATCATCCAGCCCT | |
| | Variable-a | DAKTTQPPSMDCAEGRAANLPCNHSTISGNEYVYWYRQIHSQGPQY | 818 |
| | | IIHGLKNNETNEMASLIITEDRKSSTLILPHATLRDTAVYYCIVRGGA | |
| | | DGLTFGKGTHLIIQP | |
| | CDR1-a | TISGNEY | 88 |
| | CDR2-a | GLKNN | 89 |
| | CDR3-a | CIVRGGADGLTF | 819 |
| | FWR1-a | DAKTTQPPSMDCAEGRAANLPCNHS | 439 |
| | FWR2-a | VYWYRQIHSQGPQYIIH | 92 |
| | FWR3-a | ETNEMASLIITEDRKSSTLILPHATLRDTAVYY | 93 |
| | FWR4-a | GKGTHLIIQP | 363 |
| TCRB- | b-Variable | GATACTGGAGTCTCCCAGGACCCCAGACACAAGATCACAAAGAG | 820 |
| 186 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAATCGGCAACGAGATAATTCACCCCTCCACT | |
| | | TTGGGAATGGGACCAGGCTCACTGTGACAG | |
| | Variable-b | DTGVSQDPRHKITKRGQNVTFRCDPISEHNRLYWYRQTLGQGPEFLT | 821 |
| | | YFQNEAQLEKSRLLSDRFSAERPKGSFSTLEIQRTEQGDSAMYLCAS | |
| | | NRQRDNSPLHFGNGTRLTVT | |
| | CDR1-b | SEHNR | 442 |
| | CDR2-b | FQNEAQ | 443 |
| | CDR3-b | CASNRQRDNSPLHF | 822 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GNGTRLTVT | 150 |
| TCRA- | a-Variable | GCCCAGAAGATAACTCAAACCCAACCAGGAATGTTCGTGCAGGA | 823 |
| 186 | nucleotide | AAAGGAGGCTGTGACTCTGGACTGCACATATGACACCAGTGATC | |
| | | AAAGTTATGGTCTATTCTGGTACAAGCAGCCCAGCAGTGGGGAA | |
| | | ATGATTTTTCTTATTTATCAGGGGTCTTATGACGAGCAAAATGCA | |
| | | ACAGAAGGTCGCTACTCATTGAATTTCCAGAAGGCAAGAAAATC | |
| | | CGCCAACCTTGTCATCTCCGCTTCACAACTGGGGGACTCAGCAAT | |
| | | GTATTTCTGTGCAATGAGAGAGTGGTTTTCAGATGGCCAGAAGCT | |
| | | GCTCTTTGCAAGGGGGACCATGTTAAAGGTGGATCTTA | |
| | Variable-a | AQKITQTQPGMFVQEKEAVTLDCTYDTSDQSYGLFWYKQPSSGEMI | 824 |
| | | FLIYQGSYDEQNATEGRYSLNFQKARKSANL VISASQLGDSAMYFC | |
| | | AMREWFSDGQKLLFARGTMLKVDL | |
| | CDR1-a | TSDQSYG | 403 |
| | CDR2-a | QGSYDEQN | 404 |
| | CDR3-a | CAMREWFSDGQKLLF | 825 |
| | FWR1-a | AQKITQTQPGMFVQEKEAVTLDCTYD | 406 |
| | FWR2-a | LFWYKQPSSGEMIFLIY | 407 |
| | FWR3-a | ATEGRYSLNFQKARKSANLVISASQLGDSAMYF | 408 |
| | FWR4-a | ARGTMLKVDL | 392 |
| TCRB- | b-Variable | GATGCTGATGTTACCCAGACCCCAAGGAATAGGATCACAAAGAC | 826 |
| 187 | nucleotide | AGGAAAGAGGATTATGCTGGAATGTTCTCAGACTAAGGGTCATG | |
| | | ATAGAATGTACTGGTATCGACAAGACCCAGGACTGGGCCTACGG | |
| | | TTGATCTATTACTCCTTTGATGTCAAAGATATAAACAAAGGAGAG | |
| | | ATCTCTGATGGATACAGTGTCTCTCGACAGGCACAGGCTAAATTC | |
| | | TCCCTGTCCCTAGAGTCTGCCATCCCCAACCAGACAGCTCTTTACT | |
| | | TCTGTGCCACCAGTGATGGTCAAGGGAAGCAGCCCCAGCATTTTG | |
| | | GTGATGGGACTCGACTCTCCATCCTAG | |
| | Variable-b | DADVTQTPRNRITKTGKRIMLECSQTKGHDRMYWYRQDPGLGLRLI | 827 |
| | | YYSFDVKDINKGEISDGYSVSRQAQAKFSLSLESAIPNQTALYFCATS | |
| | | DGQGKQPQHFGDGTRLSIL | |
| | CDR1-b | KGHDR | 21 |
| | CDR2-b | SFDVKD | 22 |
| | CDR3-b | CATSDGQGKQPQHF | 828 |
| | FWR1-b | DADVTQTPRNRITKTGKRIMLECSQT | 24 |
| | FWR2-b | MYWYRQDPGLGLRLIYY | 25 |
| | FWR3-b | INKGEISDGYSVSRQAQAKFSLSLESAIPNQTALYF | 26 |
| | FWR4-b | GDGTRLSIL | 27 |
| TCRA- | a-Variable | AGCCAAAAGATAGAACAGAATTCCGAGGCCCTGAACATTCAGGA | 829 |
| 187 | nucleotide | GGGTAAAACGGCCACCCTGACCTGCAACTATACAAACTATTCCCC | |
| | | AGCATACTTACAGTGGTACCGACAAGATCCAGGAAGAGGCCCTG | |
| | | TTTTCTTGCTACTCATACGTGAAAATGAGAAAGAAAAAAGGAAA | |
| | | GAAAGACTGAAGGTCACCTTTGATACCACCCTTAAACAGAGTTTG | |
| | | TTTCATATCACAGCCTCCCAGCCTGCAGACTCAGCTACCTACCTCT | |
| | | GTGCTCTAGAGGGTAATGCAGGCAACATGCTCACCTTTGGAGGG | |
| | | GGAACAAGGTTAATGGTCAAACCCC | |
| | Variable-a | SQKIEQNSEALNIQEGKTATLTCNYTNYSPAYLQWYRQDPGRGPVFL | 830 |
| | | LLIRENEKEKRKERLKVTFDTTLKQSLFHITASQPADSATYLCALEGN | |
| | | AGNMLTFGGGTRLMVKP | |
| | CDR1-a | NYSPAY | 831 |
| | CDR2-a | IRENEKE | 832 |
| | CDR3-a | CALEGNAGNMLTF | 833 |
| | FWR1-a | SQKIEQNSEALNIQEGKTATLTCNYT | 834 |
| | FWR2-a | LQWYRQDPGRGPVFLLL | 835 |
| | FWR3-a | KRKERLKVTFDTTLKQSLFHITASQPADSATYL | 836 |
| | FWR4-a | GGGTRLMVKP | 141 |
| TCRB- | b-Variable | GATGGTGGAATCACTCAGTCCCCAAAGTACCTGTTCAGAAAGGA | 904 |
| 188 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTACCCCTAGGGCTAGCGGGGAGATCGCCGATGA | |
| | | GCAGTTCTTCGGGCCAGGGACACGGCTCACCGTGCTAG | |
| | Variable-b | DGGITQSPKYLFRKEGQNVTLSCEQNLNHDAMYWYRQDPGQGLRLI | 905 |
| | | YYSQIVNDFQKGDIAEGYSVSREKKESFPLTVTSAQKNPTAFYLCAS | |
| | | TPRASGEIADEQFFGPGTRLTVL | |
| | CDR1-b | LNHDA | 216 |
| | CDR2-b | SQIVND | 217 |
| | CDR3-b | CASTPRASGEIADEQFF | 906 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVL | 9 |
| TCRA- | a-Variable | GCTCAGAAGGTAACTCAAGCGCAGACTGAAATTTCTGTGGTGGA | 907 |
| 188 | nucleotide | GAAGGAGGATGTGACCTTGGACTGTGTGTATGAAACCCGTGATA | |
| | | CTACTTATTACTTATTCTGGTACAAGCAACCACCAAGTGGAGAAT | |
| | | TGGTTTTCCTTATTCGTCGGAACTCTTTTGATGAGCAAAATGAAAT | |
| | | AAGTGGTCGGTATTCTTGGAACTTCCAGAAATCCACCAGTTCCTT | |
| | | CAACTTCACCATCACAGCCTCACAAGTCGTGGACTCAGCAGTATA | |
| | | CTTCTGTGCTCTGAGTGAGGGGGGATACAACTTCAACAAATTTTA | |
| | | CTTTGGATCTGGGACCAAACTCAATGTAAAACCAA | |
| | Variable-a | AQKVTQAQTEISVVEKEDVTLDCVYETRDTTYYLFWYKQPPSGELV | 908 |
| | | FLIRRNSFDEQNEISGRYSWNFQKSTSSFNFTITASQVVDSAVYFCAL | |
| | | SEGGYNFNKFYFGSGTKLNVKP | |
| | CDR1-a | TRDTTYY | 48 |
| | CDR2-a | RNSFDEQN | 49 |
| | CDR3-a | CALSEGGYNFNKFYF | 909 |
| | FWR1-a | AQKVTQAQTEISVVEKEDVTLDCVYE | 51 |
| | FWR2-a | LFWYKQPPSGEL VFLIR | 52 |
| | FWR3-a | EISGRYSWNFQKSTSSFNFTITASQVVDSAVYF | 53 |
| | FWR4-a | GSGTKLNVKP | 910 |
| TCRB- | b-Variable | GAAGCTGGAGTTACTCAGTTCCCCAGCCACAGCGTAATAGAGAA | 911 |
| 189 | nucleotide | GGGCCAGACTGTGACTCTGAGATGTGACCCAATTTCTGGACATGA | |
| | | TAATCTTTATTGGTATCGACGTGTTATGGGAAAAGAAATAAAATT | |
| | | TCTGTTACATTTTGTGAAAGAGTCTAAACAGGATGAGTCCGGTAT | |
| | | GCCCAACAATCGATTCTTAGCTGAAAGGACTGGAGGGACGTATTC | |
| | | TACTCTGAAGGTGCAGCCTGCAGAACTGGAGGATTCTGGAGTTTA | |
| | | TTTCTGTGCCAGCAGCCAAGAAGTCAGCCGCCCACACAATGAGC | |
| | | AGTTCTTCGGGCCAGGGACACGGCTCACCGTGCTAG | |
| | Variable-b | EAGVTQFPSHSVIEKGQTVTLRCDPISGHDNLYWYRRVMGKEIKFLL | 912 |
| | | HFVKESKQDESGMPNNRFLAERTGGTYSTLKVQPAELEDSGVYFCA | |
| | | SSQEVSRPHNEQFFGPGTRLTVL | |
| | CDR1-b | SGHDN | 57 |
| | CDR2-b | FVKESK | 58 |
| | CDR3-b | CASSQEVSRPHNEQFF | 913 |
| | FWR1-b | EAGVTQFPSHSVIEKGQTVTLRCDPI | 60 |
| | FWR2-b | LYWYRRVMGKEIKFLLH | 61 |
| | FWR3-b | QDESGMPNNRFLAERTGGTYSTLKVQPAELEDSGVYF | 62 |
| | FWR4-b | GPGTRLTVL | 9 |
| TCRA- | a-Variable | GGAATACAAGTGGAGCAGAGTCCTCCAGACCTGATTCTCCAGGA | 914 |
| 189 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTGGA | |
| | | GCCTCTCACGGGAGGAGGAAACAAACTCACCTTTGGGACAGGCA | |
| | | CTCAGCTAAAAGTGGAACTCA | |
| | Variable-a | GIQVEQSPPDLILQEGANSTLRCNFSDSVNNLQWFHQNPWGQLINLF | 915 |
| | | YIPSGTKQNGRLSATTVATERYSLLYISSSQTTDSGVYFCAVEPLTGG | |
| | | GNKLTFGTGTQLKVEL | |
| | CDR1-a | DSVNN | 506 |
| | CDR2-a | IPSGT | 507 |
| | CDR3-a | CAVEPLTGGGNKLTF | 916 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GTGTQLKVEL | 456 |
| TCRB- | b-Variable | GATGTGAAAGTAACCCAGAGCTCGAGATATCTAGTCAAAAGGAC | 917 |
| 190 | nucleotide | GGGAGAGAAAGTTTTTCTGGAATGTGTCCAGGATATGGACCATG | |
| | | AAAATATGTTCTGGTATCGACAAGACCCAGGTCTGGGGCTACGGC | |
| | | TGATCTATTTCTCATATGATGTTAAAATGAAAGAAAAAGGAGATA | |
| | | TTCCTGAGGGGTACAGTGTCTCTAGAGAGAAGAAGGAGCGCTTCT | |
| | | CCCTGATTCTGGAGTCCGCCAGCACCAACCAGACATCTATGTACC | |
| | | TCTGTGCCAGCAGTTTATTACCCAGAGGGGGTGGTGGTGGCTACA | |
| | | CCTTCGGTTCGGGGACCAGGTTAACCGTTGTAG | |
| | Variable-b | DVKVTQSSRYLVKRTGEKVFLECVQDMDHENMFWYRQDPGLGLRL | 918 |
| | | IYFSYDVKMKEKGDIPEGYSVSREKKERFSLILESASTNQTSMYLCAS | |
| | | SLLPRGGGGGYTFGSGTRLTVV | |
| | CDR1-b | MDHEN | 199 |
| | CDR2-b | SYDVKM | 200 |
| | CDR3-b | CASSLLPRGGGGGYTF | 919 |
| | FWR1-b | DVKVTQSSRYLVKRTGEKVFLECVQD | 202 |
| | FWR2-b | MFWYRQDPGLGLRLIYF | 203 |
| | FWR3-b | KEKGDIPEGYSVSREKKERFSLILESASTNQTSMYL | 204 |
| | FWR4-b | GSGTRLTVV | 103 |
| TCRA- | a-Variable | AAGGACCAAGTGTTTCAGCCTTCCACAGTGGCATCTTCAGAGGGA | 920 |
| 190 | nucleotide | GCTGTGGTGGAAATCTTCTGTAATCACTCTGTGTCCAATGCTTAC | |
| | | AACTTCTTCTGGTACCTTCACTTCCCGGGATGTGCACCAAGACTC | |
| | | CTTGTTAAAGGCTCAAAGCCTTCTCAGCAGGGACGATACAACATG | |
| | | ACCTATGAACGGTTCTCTTCATCGCTGCTCATCCTCCAGGTGCGG | |
| | | GAGGCAGATGCTGCTGTTTACTACTGTGCTGTTGCAGATTCAGGA | |
| | | AACACACCTCTTGTCTTTGGAAAGGGCACAAGACTTTCTGTGATT | |
| | | GCAA | |
| | Variable-a | KDQVFQPSTVASSEGAVVEIFCNHSVSNAYNFFWYLHFPGCAPRLLV | 921 |
| | | KGSKPSQQGRYNMTYERFSSSLLILQVREADAAVYYCAVADSGNTP | |
| | | LVFGKGTRLSVIA | |
| | CDR1-a | VSNAYN | 236 |
| | CDR2-a | GSKP | 237 |
| | CDR3-a | CAVADSGNTPLVF | 922 |
| | FWR1-a | KDQVFQPSTVASSEGAVVEIFCNHS | 239 |
| | FWR2-a | FFWYLHFPGCAPRLLVK | 240 |
| | FWR3-a | SQQGRYNMTYERFSSSLLILQVREADAAVYY | 241 |
| | FWR4-a | GKGTRLSVIA | 546 |
| TCRB- | b-Variable | AATGCTGGTGTCACTCAGACCCCAAAATTCCAGGTCCTGAAGACA | 923 |
| 191 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTTACGCTAGCCTCACGGACCAGTTCTTCGGGCC | |
| | | AGGGACACGGCTCACCGTGCTAG | |
| | Variable-b | NAGVTQTPKFQVLKTGQSMTLQCAQDMNHEYMSWYRQDPGMGLR | 924 |
| | | LIHYSVGAGITDQGEVPNGYNVSRSTTEDFPLRLLSAAPSQTSVYFCA | |
| | | SSYASLTDQFFGPGTRLTVL | |
| | CDR1-b | MNHEY | 252 |
| | CDR2-b | SVGAGI | 253 |
| | CDR3-b | CASSYASLTDQFF | 925 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GPGTRLTVL | 9 |
| TCRA- | a-Variable | ACCCAGCTGCTGGAGCAGAGCCCTCAGTTTCTAAGCATCCAAGAG | 926 |
| 191 | nucleotide | GGAGAAAATCTCACTGTGTACTGCAACTCCTCAAGTGTTTTTTCC | |
| | | AGCTTACAATGGTACAGACAGGAGCCTGGGGAAGGTCCTGTCCT | |
| | | CCTGGTGACAGTAGTTACGGGTGGAGAAGTGAAGAAGCTGAAGA | |
| | | GACTAACCTTTCAGTTTGGTGATGCAAGAAAGGACAGTTCTCTCC | |
| | | ACATCACTGCGGCCCAGCCTGGTGATACAGGCCTCTACCTCTGTG | |
| | | CAGGAGCTGAGGATGCCAGACTCATGTTTGGAGATGGAACTCAG | |
| | | CTGGTGGTGAAGCCCA | |
| | Variable-a | TQLLEQSPQFLSIQEGENLTVYCNSSSVFSSLQWYRQEPGEGPVLLVT | 927 |
| | | VVTGGEVKKLKRLTFQFGDARKDSSLHITAAQPGDTGLYLCAGAED | |
| | | ARLMFGDGTQLVVKP | |
| | CDR1-a | SVFSS | 533 |
| | CDR2-a | VVTGGEV | 534 |
| | CDR3-a | CAGAEDARLMF | 928 |
| | FWR1-a | TQLLEQSPQFLSIQEGENLTVYCNSS | 536 |
| | FWR2-a | LQWYRQEPGEGPVLLVT | 537 |
| | FWR3-a | KKLKRLTFQFGDARKDSSLHITAAQPGDTGLYL | 538 |
| | FWR4-a | GDGTQLVVKP | 230 |
| TCRB- | b-Variable | GATACTGGAGTCTCCCAGGACCCCAGACACAAGATCACAAAGAG | 929 |
| 192.1 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCTTGAGTCGGGGACAGGGGGCTTTTCAGC | |
| | | CCCAGCATTTTGGTGATGGGACTCGACTCTCCATCCTAG | |
| | Variable-b | DTGVSQDPRHKITKRGQNVTFRCDPISEHNRLYWYRQTLGQGPEFLT | 930 |
| | | YFQNEAQLEKSRLLSDRFSAERPKGSFSTLEIQRTEQGDSAMYLCASS | |
| | | LSRGQGAFQPQHFGDGTRLSIL | |
| | CDR1-b | SEHNR | 442 |
| | CDR2-b | FQNEAQ | 443 |
| | CDR3-b | CASSLSRGQGAFQPQHF | 931 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GDGTRLSIL | 27 |
| TCRB- | b-Variable | GACACTGAAGTTACCCAGACACCAAAACACCTGGTCATGGGAAT | 932 |
| 192.2 | nucleotide | GACAAATAAGAAGTCTTTGAAATGTGAACAACATATGGGGCACA | |
| | | GGGCTATGTATTGGTACAAGCAGAAAGCTAAGAAGCCACCGGAG | |
| | | CTCATGTTTGTCTACAGCTATGAGAAACTCTCTATAAATGAAAGT | |
| | | GTGCCAAGTCGCTTCTCACCTGAATGCCCCAACAGCTCTCTCTTA | |
| | | AACCTTCACCTACACGCCCTGCAGCCAGAAGACTCAGCCCTGTAT | |
| | | CTCTGCGCCAGCAGCCAGGCAACTAATGAAAAACTGTTTTTTGGC | |
| | | AGTGGAACCCAGCTCTCTGTCTTGG | |
| | Variable-b | DTEVTQTPKHLVMGMTNKKSLKCEQHMGHRAMYWYKQKAKKPP | 933 |
| | | ELMFVYSYEKLSINESVPSRFSPECPNSSLLNLHLHALQPEDSALYLC | |
| | | ASSQATNEKLFFGSGTQLSVL | |
| | CDR1-b | MGHRA | 639 |
| | CDR2-b | YSYEKL | 640 |
| | CDR3-b | CASSQATNEKLFF | 934 |
| | FWR1-b | DTEVTQTPKHLVMGMTNKKSLKCEQH | 642 |
| | FWR2-b | MYWYKQKAKKPPELMFV | 643 |
| | FWR3-b | SINESVPSRFSPECPNSSLLNLHLHALQPEDSALYL | 644 |
| | FWR4-b | GSGTQLSVL | 45 |
| TCRA- | a-Variable | GGAGAGAATGTGGAGCAGCATCCTTCAACCCTGAGTGTCCAGGA | 935 |
| 192 | nucleotide | GGGAGACAGCGCTGTTATCAAGTGTACTTATTCAGACAGTGCCTC | |
| | | AAACTACTTCCCTTGGTATAAGCAAGAACTTGGAAAAGGACCTCA | |
| | | GCTTATTATAGACATTCGTTCAAATGTGGGCGAAAAGAAAGACC | |
| | | AACGAATTGCTGTTACATTGAACAAGACAGCCAAACATTTCTCCC | |
| | | TGCACATCACAGAGACCCAACCTGAAGACTCGGCTGTCTACTTCT | |
| | | GTGCAGCATATGGTACTAGCTATGGAAAGCTGACATTTGGACAA | |
| | | GGGACCATCTTGACTGTCCATCCAA | |
| | Variable-a | GENVEQHPSTLSVQEGDSAVIKCTYSDSASNYFPWYKQELGKGPQLI | 936 |
| | | IDIRSNVGEKKDQRIA VTLNKTAKHFSLHITETQPEDSAVYFCAAYGT | |
| | | SYGKLTFGQGTILTVHP | |
| | CDR1-a | DSASNY | 207 |
| | CDR2-a | IRSNVGE | 208 |
| | CDR3-a | CAAYGTSYGKLTF | 937 |
| | FWR1-a | GENVEQHPSTLSVQEGDSAVIKCTYS | 210 |
| | FWR2-a | FPWYKQELGKGPQLIID | 374 |
| | FWR3-a | KKDQRIAVTLNKTAKHFSLHITETQPEDSAVYF | 212 |
| | FWR4-a | GQGTILTVHP | 76 |
| TCRB- | b-Variable | GGTGCTGTCGTCTCTCAACATCCGAGCTGGGTTATCTGTAAGAGT | 938 |
| 193 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTTGCCGTGACAGGGGCTACACTGAAG | |
| | | CTTTCTTTGGACAAGGCACCAGACTCACAGTTGTAG | |
| | Variable-b | GAVVSQHPSWVICKSGTSVKIECRSLDFQATTMFWYRQFPKQSLML | 939 |
| | | MATSNEGSKATYEQGVEKDKFLINHASLTLSTLTVTSAHPEDSSFYIC | |
| | | SACRDRGYTEAFFGQGTRLTVV | |
| | CDR1-b | DFQATT | 79 |
| | CDR2-b | SNEGSKA | 80 |
| | CDR3-b | CSACRDRGYTEAFF | 940 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GQGTRLTVV | 246 |
| TCRA- | a-Variable | GCTCAGACAGTCACTCAGTCTCAACCAGAGATGTCTGTGCAGGAG | 941 |
| 193 | nucleotide | GCAGAGACCGTGACCCTGAGCTGCACATATGACACCAGTGAGAG | |
| | | TGATTATTATTTATTCTGGTACAAGCAGCCTCCCAGCAGGCAGAT | |
| | | GATTCTCGTTATTCGCCAAGAAGCTTATAAGCAACAGAATGCAAC | |
| | | AGAGAATCGTTTCTCTGTGAACTTCCAGAAAGCAGCCAAATCCTT | |
| | | CAGTCTCAAGATCTCAGACTCACAGCTGGGGGATGCCGCGATGTA | |
| | | TTTCTGTGCTTATAGGTTCAATAACAATGACATGCGCTTTGGAGC | |
| | | AGGGACCAGACTGACAGTAAAACCAA | |
| | Variable-a | AQTVTQSQPEMSVQEAETVTLSCTYDTSESDYYLFWYKQPPSRQMI | 942 |
| | | LVIRQEAYKQQNATENRFSVNFQKAAKSFSLKISDSQLGDAAMYFC | |
| | | AYRFNNNDMRFGAGTRLTVKP | |
| | CDR1-a | TSESDYY | 286 |
| | CDR2-a | QEAYKQQN | 287 |
| | CDR3-a | CAYRFNNNDMRF | 943 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDAAMYF | 291 |
| | FWR4-a | GAGTRLTVKP | 685 |
| TCRB- | b-Variable | GATGTGAAAGTAACCCAGAGCTCGAGATATCTAGTCAAAAGGAC | 679 |
| 194 | nucleotide | GGGAGAGAAAGTTTTTCTGGAATGTGTCCAGGATATGGACCATG | |
| | | AAAATATGTTCTGGTATCGACAAGACCCAGGTCTGGGGCTACGGC | |
| | | TGATCTATTTCTCATATGATGTTAAAATGAAAGAAAAAGGAGATA | |
| | | TTCCTGAGGGGTACAGTGTCTCTAGAGAGAAGAAGGAGCGCTTCT | |
| | | CCCTGATTCTGGAGTCCGCCAGCACCAACCAGACATCTATGTACC | |
| | | TCTGTGCCAGCAGTTGGACAGAAGAGACCTACGAGCAGTACTTC | |
| | | GGGCCGGGCACCAGGCTCACGGTCACAG | |
| | Variable-b | DVKVTQSSRYLVKRTGEKVFLECVQDMDHENMFWYRQDPGLGLRL | 680 |
| | | IYFSYDVKMKEKGDIPEGYSVSREKKERFSLILESASTNQTSMYLCAS | |
| | | SWTEETYEQYFGPGTRLTVT | |
| | CDR1-b | MDHEN | 199 |
| | CDR2-b | SYDVKM | 200 |
| | CDR3-b | CASSWTEETYEQYF | 681 |
| | FWR1-b | DVKVTQSSRYLVKRTGEKVFLECVQD | 202 |
| | FWR2-b | MFWYRQDPGLGLRLIYF | 203 |
| | FWR3-b | KEKGDIPEGYSVSREKKERFSLILESASTNQTSMYL | 204 |
| | FWR4-b | GPGTRLTVT | 85 |
| TCRA- | a-Variable | GCTCAGTCAGTGGCTCAGCCGGAAGATCAGGTCAACGTTGCTGA | 682 |
| 194 | nucleotide | AGGGAATCCTCTGACTGTGAAATGCACCTATTCAGTCTCTGGAAA | |
| | | CCCTTATCTTTTTTGGTATGTTCAATACCCCAACCGAGGCCTCCAG | |
| | | TTCCTTCTGAAATACATCACAGGGGATAACCTGGTTAAAGGCAGC | |
| | | TATGGCTTTGAAGCTGAATTTAACAAGAGCCAAACCTCCTTCCAC | |
| | | CTGAAGAAACCATCTGCCCTTGTGAGCGACTCCGCTTTGTACTTC | |
| | | TGTGCTGTGAGAGCGTACAATAACAATGACATGCGCTTTGGAGCA | |
| | | GGGACCAGACTGACAGTAAAACCAA | |
| | Variable-a | AQSVAQPEDQVNVAEGNPLTVKCTYSVSGNPYLFWYVQYPNRGLQ | 683 |
| | | FLLKYITGDNLVKGSYGFEAEFNKSQTSFHLKKPSALVSDSALYFCA | |
| | | VRAYNNNDMRFGAGTRLTVKP | |
| | CDR1-a | VSGNPY | 427 |
| | CDR2-a | YITGDNLV | 428 |
| | CDR3-a | CAVRAYNNNDMRF | 684 |
| | FWR1-a | AQSVAQPEDQVNVAEGNPLTVKCTYS | 430 |
| | FWR2-a | LFWYVQYPNRGLQFLLK | 431 |
| | FWR3-a | KGSYGFEAEFNKSQTSFHLKKPSALVSDSALYF | 432 |
| | FWR4-a | GAGTRLTVKP | 685 |
| TCRB- | b-Variable | GATTCTGGAGTCACACAAACCCCAAAGCACCTGATCACAGCAAC | 1019 |
| 195 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCCCGAAGGACGTCAGTACTGGGGCCAACGTCC | |
| | | TGACTTTCGGGGCCGGCAGCAGGCTGACCGTGCTGG | |
| | Variable-b | DSGVTQTPKHLITATGQRVTLRCSPRSGDLSVYWYQQSLDQGLQFLI | 1020 |
| | | QYYNGEERAKGNILERFSAQQFPDLHSELNLSSLELGDSALYFCASSP | |
| | | KDVSTGANVLTFGAGSRLTVL | |
| | CDR1-b | SGDLS | 383 |
| | CDR2-b | YYNGEE | 384 |
| | CDR3-b | CASSPKDVSTGANVLTF | 1021 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GAGSRLTVL | 816 |
| TCRA- | a-Variable | GATGCTAAGACCACACAGCCAAATTCAATGGAGAGTAACGAAGA | 1022 |
| 195 | nucleotide | AGAGCCTGTTCACTTGCCTTGTAACCACTCCACAATCAGTGGAAC | |
| | | TGATTACATACATTGGTATCGACAGCTTCCCTCCCAGGGTCCAGA | |
| | | GTACGTGATTCATGGTCTTACAAGCAATGTGAACAACAGAATGGC | |
| | | CTCTCTGGCAATCGCTGAAGACAGAAAGTCCAGTACCTTGATCCT | |
| | | GCACCGTGCTACCTTGAGAGATGCTGCTGTGTACTACTGCATCCT | |
| | | GAAAACCTCCTACGACAAGGTGATATTTGGGCCAGGGACAAGCT | |
| | | TATCAGTCATTCCAA | |
| | Variable-a | DAKTTQPNSMESNEEEPVHLPCNHSTISGTDYIHWYRQLPSQGPEYV | 1023 |
| | | IHGLTSNVNNRMASLAIAEDRKSSTLILHRATLRDAAVYYCILKTSY | |
| | | DKVIFGPGTSLSVIP | |
| | CDR1-a | TISGTDY | 153 |
| | CDR2-a | GLTSN | 154 |
| | CDR3-a | CILKTSYDKVIF | 1024 |
| | FWR1-a | DAKTTQPNSMESNEEEPVHLPCNHS | 156 |
| | FWR2-a | IHWYRQLPSQGPEYVIH | 157 |
| | FWR3-a | VNNRMASLAIAEDRKSSTLILHRATLRDAAVYY | 158 |
| | FWR4-a | GPGTSLSVIP | 1025 |
| TCRB- | b-Variable | GAAGCCCAAGTGACCCAGAACCCAAGATACCTCATCACAGTGAC | 1026 |
| 196 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTTTAGAGGGTAGCGGAAGTGCCGGGGAGCTG | |
| | | TTTTTTGGAGAAGGCTCTAGGCTGACCGTACTGG | |
| | Variable-b | EAQVTQNPRYLITVTGKKLTVTCSQNMNHEYMSWYRQDPGLGLRQ | 1027 |
| | | IYYSMNVEVTDKGDVPEGYKVSRKEKRNFPLILESPSPNQTSLYFCA | |
| | | SSLEGSGSAGELFFGEGSRLTVL | |
| | CDR1-b | MNHEY | 252 |
| | CDR2-b | SMNVEV | 366 |
| | CDR3-b | CASSLEGSGSAGELFF | 1028 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GEGSRLTVL | 63 |
| TCRA- | a-Variable | AGCCAAAAGATAGAACAGAATTCCGAGGCCCTGAACATTCAGGA | 1029 |
| 196 | nucleotide | GGGTAAAACGGCCACCCTGACCTGCAACTATACAAACTATTCTCC | |
| | | AGCATACTTACAGTGGTACCGACAAGATCCAGGAAGAGGCCCTG | |
| | | TTTTCTTGCTACTCATACGTGAAAATGAGAAAGAAAAAAGGAAA | |
| | | GAAAGACTGAAGGTCACCTTTGATACCACCCTTAAACAGAGTTTG | |
| | | TTTCATATCACAGCCTCCCAGCCTGCAGACTCAGCTACCTACCTCT | |
| | | GTGCTCTAGAAGAAACTGACAGCTGGGGGAAATTGCAGTTTGGA | |
| | | GCAGGGACCCAGGTTGTGGTCACCCCAG | |
| | Variable-a | SQKIEQNSEALNIQEGKTATLTCNYTNYSPAYLQWYRQDPGRGPVFL | 1030 |
| | | LLIRENEKEKRKERLKVTFDTTLKQSLFHITASQPADSATYLCALEET | |
| | | DSWGKLQFGAGTQVVVTP | |
| | CDR1-a | NYSPAY | 831 |
| | CDR2-a | IRENEKE | 832 |
| | CDR3-a | CALEETDSWGKLQF | 1031 |
| | FWR1-a | SQKIEQNSEALNIQEGKTATLTCNYT | 834 |
| | FWR2-a | LQWYRQDPGRGPVFLLL | 835 |
| | FWR3-a | KRKERLKVTFDTTLKQSLFHITASQPADSATYL | 836 |
| | FWR4-a | GAGTQVVVTP | 54 |
| TCRB- | b-Variable | GATTCTGGAGTCACACAAACCCCAAAGCACCTGATCACAGCAAC | 1032 |
| 197 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCCGGCGAAGCTTGAGCTCCTACAATGAGCAGTTCTT | |
| | | CGGGCCAGGGACACGGCTCACCGTGCTAG | |
| | Variable-b | DSGVTQTPKHLITATGQRVTLRCSPRSGDLSVYWYQQSLDQGLQFLI | 1033 |
| | | QYYNGEERAKGNILERFSAQQFPDLHSELNLSSLELGDSALYFCASR | |
| | | RSLSSYNEQFFGPGTRLTVL | |
| | CDR1-b | SGDLS | 383 |
| | CDR2-b | YYNGEE | 384 |
| | CDR3-b | CASRRSLSSYNEQFF | 1034 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GPGTRLTVL | 9 |
| TCRA- | a-Variable | GACCAGCAAGTTAAGCAAAATTCACCATCCCTGAGCGTCCAGGA | 1035 |
| 197 | nucleotide | AGGAAGAATTTCTATTCTGAACTGTGACTATACTAACAGCATGTT | |
| | | TGATTATTTCCTATGGTACAAAAAATACCCTGCTGAAGGTCCTAC | |
| | | ATTCCTGATATCTATAAGTTCCATTAAGGATAAAAATGAAGATGG | |
| | | AAGATTCACTGTTTTCTTAAACAAAAGTGCCAAGCACCTCTCTCT | |
| | | GCACATTGTGCCCTCCCAGCCTGGAGACTCTGCAGTGTACTTCTG | |
| | | TGCAGCAACCTATAATAATGCAGGCAACATGCTCACCTTTGGAGG | |
| | | GGGAACAAGGTTAATGGTCAAACCCC | |
| | Variable-a | DQQVKQNSPSLSVQEGRISILNCDYTNSMFDYFLWYKKYPAEGPTFL | 1036 |
| | | ISISSIKDKNEDGRFTVFLNKSAKHLSLHIVPSQPGDSAVYFCAATYN | |
| | | NAGNMLTFGGGTRLMVKP | |
| | CDR1-a | NSMFDY | 303 |
| | CDR2-a | ISSIKDK | 304 |
| | CDR3-a | CAATYNNAGNMLTF | 1037 |
| | FWR1-a | DQQVKQNSPSLSVQEGRISILNCDYT | 306 |
| | FWR2-a | FLWYKKYPAEGPTFLIS | 307 |
| | FWR3-a | NEDGRFTVFLNKSAKHLSLHIVPSQPGDSAVYF | 308 |
| | FWR4-a | GGGTRLMVKP | 141 |
| TCRB- | b-Variable | GATTCTGGAGTCACACAAACCCCAAAGCACCTGATCACAGCAAC | 1038 |
| 198 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCGTAGGGAGTGTGGGGGGACTAGCGGACTTCA | |
| | | ATGAGCAGTTCTTCGGGCCAGGGACACGGCTCACCGTGCTAG | |
| | Variable-b | DSGVTQTPKHLITATGQRVTLRCSPRSGDLSVYWYQQSLDQGLQFLI | 1039 |
| | | QYYNGEERAKGNILERFSAQQFPDLHSELNLSSLELGDSALYFCASS | |
| | | VGSVGGLADFNEQFFGPGTRLTVL | |
| | CDR1-b | SGDLS | 383 |
| | CDR2-b | YYNGEE | 384 |
| | CDR3-b | CASSVGSVGGLADFNEQFF | 1040 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GPGTRLTVL | 9 |
| TCRA- | a-Variable | GCCCAGAAGATAACTCAAACCCAACCAGGAATGTTCGTGCAGGA | 1041 |
| 198 | nucleotide | AAAGGAGGCTGTGACTCTGGACTGCACATATGACACCAGTGATC | |
| | | AAAGTTATGGTCTATTCTGGTACAAGCAGCCCAGCAGTGGGGAA | |
| | | ATGATTTTTCTTATTTATCAGGGGTCTTATGACGAGCAAAATGCA | |
| | | ACAGAAGGTCGCTACTCATTGAATTTCCAGAAGGCAAGAAAATC | |
| | | CGCCAACCTTGTCATCTCCGCTTCACAACTGGGGGACTCAGCAAT | |
| | | GTATTTCTGTGCAATGAGAGAGGGCTGGGTTAGTGACATGCGCTT | |
| | | TGGAGCAGGGACCAGACTGACAGTAAAACCAA | |
| | Variable-a | AQKITQTQPGMFVQEKEAVTLDCTYDTSDQSYGLFWYKQPSSGEMI | 1042 |
| | | FLIYQGSYDEQNATEGRYSLNFQKARKSANLVISASQLGDSAMYFC | |
| | | AMREGWVSDMRFGAGTRLTVKP | |
| | CDR1-a | TSDQSYG | 403 |
| | CDR2-a | QGSYDEQN | 404 |
| | CDR3-a | CAMREGWVSDMRF | 1043 |
| | FWR1-a | AQKITQTQPGMFVQEKEAVTLDCTYD | 406 |
| | FWR2-a | LFWYKQPSSGEMIFLIY | 407 |
| | FWR3-a | ATEGRYSLNFQKARKSANLVISASQLGDSAMYF | 408 |
| | FWR4-a | GAGTRLTVKP | 685 |
| TCRB- | b-Variable | AATGCTGGTGTCACTCAGACCCCAAAATTCCGGGTCCTGAAGACA | 1044 |
| 199 | nucleotide | GGACAGAGCATGACACTGCTGTGTGCCCAGGATATGAACCATGA | |
| | | ATACATGTACTGGTATCGACAAGACCCAGGCATGGGGCTGAGGC | |
| | | TGATTCATTACTCAGTTGGTGAGGGTACAACTGCCAAAGGAGAG | |
| | | GTCCCTGATGGCTACAATGTCTCCAGATTAAAAAAACAGAATTTC | |
| | | CTGCTGGGGTTGGAGTCGGCTGCTCCCTCCCAAACATCTGTGTAC | |
| | | TTCTGTGCCAGCAGTTCCCCTTTCTACGAGCAGTACTTCGGGCCG | |
| | | GGCACCAGGCTCACGGTCACAG | |
| | Variable-b | NAGVTQTPKFRVLKTGQSMTLLCAQDMNHEYMYWYRQDPGMGLR | 1045 |
| | | LIHYSVGEGTTAKGEVPDGYNVSRLKKQNFLLGLESAAPSQTSVYFC | |
| | | ASSSPFYEQYFGPGTRLTVT | |
| | CDR1-b | MNHEY | 252 |
| | CDR2-b | SVGEGT | 1046 |
| | CDR3-b | CASSSPFYEQYF | 1047 |
| | FWR1-b | NAGVTQTPKFRVLKTGQSMTLLCAQD | 1048 |
| | FWR2-b | MYWYRQDPGMGLRLIHY | 1049 |
| | FWR3-b | TAKGEVPDGYNVSRLKKQNFLLGLESAAPSQTSVYF | 1050 |
| | FWR4-b | GPGTRLTVT | 85 |
| TCRA- | a-Variable | GGAATACAAGTGGAGCAGAGTCCTCCAGACCTGATTCTCCAGGA | 1051 |
| 199 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTGGA | |
| | | AGAATGGGGAAGCCAAGGAAATCTCATCTTTGGAAAAGGCACTA | |
| | | AACTCTCTGTTAAACCAA | |
| | Variable-a | GIQVEQSPPDLILQEGANSTLRCNFSDSVNNLQWFHQNPWGQLINLF | 1052 |
| | | YIPSGTKQNGRLSATTVATERYSLLYISSSQTTDSGVYFCAVEEWGS | |
| | | QGNLIFGKGTKLSVKP | |
| | CDR1-a | DSVNN | 506 |
| | CDR2-a | IPSGT | 507 |
| | CDR3-a | CAVEEWGSQGNLIF | 1053 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GKGTKLSVKP | 124 |
| TCRB- | b-Variable | GAAACGGGAGTTACGCAGACACCAAGACACCTGGTCATGGGAAT | 1054 |
| 200 | nucleotide | GACAAATAAGAAGTCTTTGAAATGTGAACAACATCTGGGTCATA | |
| | | ACGCTATGTATTGGTACAAGCAAAGTGCTAAGAAGCCACTGGAG | |
| | | CTCATGTTTGTCTACAGTCTTGAAGAACGGGTTGAAAACAACAGT | |
| | | GTGCCAAGTCGCTTCTCACCTGAATGCCCCAACAGCTCTCACTTA | |
| | | TTCCTTCACCTACACACCCTGCAGCCAGAAGACTCGGCCCTGTAT | |
| | | CTCTGCGCCAGCAGCCAAGGAAGGGCGTTTCTGGGCCAGGGGTT | |
| | | GTACGAGCAGTACTTCGGGCCGGGCACCAGGCTCACGGTCACAG | |
| | Variable-b | ETGVTQTPRHLVMGMTNKKSLKCEQHLGHNAMYWYKQSAKKPLE | 1055 |
| | | LMFVYSLEERVENNSVPSRFSPECPNSSHLFLHLHTLQPEDSALYLCA | |
| | | SSQGRAFLGQGLYEQYFGPGTRLTVT | |
| | CDR1-b | LGHNA | 169 |
| | CDR2-b | YSLEER | 706 |
| | CDR3-b | CASSQGRAFLGQGLYEQYF | 1056 |
| | FWR1-b | ETGVTQTPRHLVMGMTNKKSLKCEQH | 172 |
| | FWR2-b | MYWYKQSAKKPLELMFV | 173 |
| | FWR3-b | VENNSVPSRFSPECPNSSHLFLHLHTLQPEDSALYL | 708 |
| | FWR4-b | GPGTRLTVT | 85 |
| TCRA- | a-Variable | ACCCAGCTGCTGGAGCAGAGCCCTCAGTTTCTAAGCATCCAAGAG | 1057 |
| 200 | nucleotide | GGAGAAAATCTCACTGTGTACTGCAACTCCTCAAGTGTTTTTTCC | |
| | | AGCTTACAATGGTACAGACAGGAGCCTGGGGAAGGTCCTGTCCT | |
| | | CCTGGTGACAGTAGTTACGGGTGGAGAAGTGAAGAAGCTGAAGA | |
| | | GACTAACCTTTCAGTTTGGTGATGCAAGAAAGGACAGTTCTCTCC | |
| | | ACATCACTGCAGCCCAGCCTGGTGATACAGGCCTCTACCTCTGTG | |
| | | CAGGAGTAGGAAGAGATGACAAGATCATCTTTGGAAAAGGGACA | |
| | | CGACTTCATATTCTCCCCA | |
| | Variable-a | TQLLEQSPQFLSIQEGENLTVYCNSSSVFSSLQWYRQEPGEGPVLLVT | 1058 |
| | | VVTGGEVKKLKRLTFQFGDARKDSSLHITAAQPGDTGLYLCAGVGR | |
| | | DDKIIFGKGTRLHILP | |
| | CDR1-a | SVFSS | 533 |
| | CDR2-a | VVTGGEV | 534 |
| | CDR3-a | CAGVGRDDKIIF | 1059 |
| | FWR1-a | TQLLEQSPQFLSIQEGENLTVYCNSS | 536 |
| | FWR2-a | LQWYRQEPGEGPVLLVT | 537 |
| | FWR3-a | KKLKRLTFQFGDARKDSSLHITAAQPGDTGLYL | 538 |
| | FWR4-a | GKGTRLHILP | 607 |
| TCRB- | b-Variable | GGTGCTGTCGTCTCTCAACATCCGAGCAGGGTTATCTGTAAGAGT | 1060 |
| 201 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGAGATCGGGGGGCCTACGAGCAGT | |
| | | ACTTCGGGCCGGGCACCAGGCTCACGGTCACAG | |
| | Variable-b | GAVVSQHPSRVICKSGTSVKIECRSLDFQATTMFWYRQFPKQSLML | 1061 |
| | | MATSNEGSKATYEQGVEKDKFLINHASLTLSTLTVTSAHPEDSSFYIC | |
| | | SARDRGAYEQYFGPGTRLTVT | |
| | CDR1-b | DFQATT | 79 |
| | CDR2-b | SNEGSKA | 80 |
| | CDR3-b | CSARDRGAYEQYF | 1062 |
| | FWR1-b | GAVVSQHPSRVICKSGTSVKIECRSL | 1063 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVT | 85 |
| TCRA- | a-Variable | GGAAATTCAGTGACCCAGATGGAAGGGCCAGTGACTCTCTCAGA | 1064 |
| 201 | nucleotide | AGAGGCCTTCCTGACTATAAACTGCACGTACACAGCCACAGGAT | |
| | | ACCCTTCCCTTTTCTGGTATGTCCAATATCCTGGAGAAGGTCTACA | |
| | | GCTCCTCCTGAAAGCCACGAAGGCTGATGACAAGGGAAGCAACA | |
| | | AAGGTTTTGAAGCCACATACCGTAAAGAAACCACTTCTTTCCACT | |
| | | TGGAGAAAGGCTCAGTTCAAGTGTCAGACTCAGCGGTGTACTTCT | |
| | | GTGCTCTGAGTGATCTGAATAACAATGCCAGACTCATGTTTGGAG | |
| | | ATGGAACTCAGCTGGTGGTGAAGCCCA | |
| | Variable-a | GNSVTQMEGPVTLSEEAFLTINCTYTATGYPSLFWYVQYPGEGLQLL | 1065 |
| | | LKATKADDKGSNKGFEATYRKETTSFHLEKGSVQVSDSAVYFCALS | |
| | | DLNNNARLMFGDGTQLVVKP | |
| | CDR1-a | ATGYPS | 106 |
| | CDR2-a | ATKADDK | 107 |
| | CDR3-a | CALSDLNNNARLMF | 1066 |
| | FWR1-a | GNSVTQMEGPVTLSEEAFLTINCTYT | 724 |
| | FWR2-a | LFWYVQYPGEGLQLLLK | 110 |
| | FWR3-a | GSNKGFEATYRKETTSFHLEKGSVQVSDSAVYF | 111 |
| | FWR4-a | GDGTQLVVKP | 230 |
| TCRB- | b-Variable | GAACCTGAAGTCACCCAGACTCCCAGCCATCAGGTCACACAGAT | 1067 |
| 202 | nucleotide | GGGACAGGAAGTGATCTTGCGCTGTGTCCCCATCTCTAATCACTT | |
| | | ATACTTCTATTGGTACAGACAAATCTTGGGGCAGAAAGTCGAGTT | |
| | | TCTGGTTTCCTTTTATAATAATGAAATCTCAGAGAAGTCTGAAAT | |
| | | ATTCGATGATCAATTCTCAGTTGAAAGGCCTGATGGATCAAATTT | |
| | | CACTCTGAAGATCCGGTCCACAAAGCTGGAGGACTCAGCCATGT | |
| | | ACTTCTGTGCCAGCAGTGCGAGCAGCGGGGGGTTCTACAATGAG | |
| | | CAGTTCTTCGGGCCAGGGACACGGCTCACCGTGCTAG | |
| | Variable-b | EPEVTQTPSHQVTQMGQEVILRCVPISNHLYFYWYRQILGQKVEFLV | 1068 |
| | | SFYNNEISEKSEIFDDQFSVERPDGSNFTLKIRSTKLEDSAMYFCASSA | |
| | | SSGGFYNEQFFGPGTRLTVL | |
| | CDR1-b | SNHLY | 525 |
| | CDR2-b | FYNNEI | 526 |
| | CDR3-b | CASSASSGGFYNEQFF | 1069 |
| | FWR1-b | EPEVTQTPSHQVTQMGQEVILRCVPI | 528 |
| | FWR2-b | FYWYRQILGQKVEFLVS | 529 |
| | FWR3-b | SEKSEIFDDQFSVERPDGSNFTLKIRSTKLEDSAMYF | 530 |
| | FWR4-b | GPGTRLTVL | 9 |
| TCRA- | a-Variable | GCCCAGTCTGTGAGCCAGCATAACCACCACGTAATTCTCTCTGAA | 1070 |
| 202 | nucleotide | GCAGCCTCACTGGAGTTGGGATGCAACTATTCCTATGGTGGAACT | |
| | | GTTAATCTCTTCTGGTATGTCCAGTACCCTGGTCAACACCTTCAGC | |
| | | TTCTCCTCAAGTACTTTTCAGGGGATCCACTGGTTAAAGGCATCA | |
| | | AGGGCTTTGAGGCTGAATTTATAAAGAGTAAATTCTCCTTTAATC | |
| | | TGAGGAAACCCTCTGTGCAGTGGAGTGACACAGCTGAGTACTTCT | |
| | | GTGCCGTGAATGGGGCAGGAGGAGGTGCTGACGGACTCACCTTT | |
| | | GGCAAAGGGACTCATCTAATCATCCAGCCCT | |
| | Variable-a | AQSVSQHNHHVILSEAASLELGCNYSYGGTVNLFWYVQYPGQHLQL | 1071 |
| | | LLKYFSGDPLVKGIKGFEAEFIKSKFSFNLRKPSVQWSDTAEYFCAV | |
| | | NGAGGGADGLTFGKGTHLIIQP | |
| | CDR1-a | YGGTVN | 1072 |
| | CDR2-a | YFSGDPLV | 1073 |
| | CDR3-a | CAVNGAGGGADGLTF | 1074 |
| | FWR1-a | AQSVSQHNHHVILSEAASLELGCNYS | 1075 |
| | FWR2-a | LFWYVQYPGQHLQLLLK | 1076 |
| | FWR3-a | KGIKGFEAEFIKSKFSFNLRKPSVQWSDTAEYF | 1077 |
| | FWR4-a | GKGTHLIIQP | 363 |
| TCRB- | b-Variable | GATACTGGAGTCTCCCAGGACCCCAGACACAAGATCACAAAGAG | 1078 |
| 203 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCTTAGTGTCCGCGGTAGGGAACATTCAGT | |
| | | ACTTCGGCGCCGGGACCCGGCTCTCAGTGCTGG | |
| | Variable-b | DTGVSQDPRHKITKRGQNVTFRCDPISEHNRLYWYRQTLGQGPEFLT | 1079 |
| | | YFQNEAQLEKSRLLSDRFSAERPKGSFSTLEIQRTEQGDSAMYLCASS | |
| | | LVSAVGNIQYFGAGTRLSVL | |
| | CDR1-b | SEHNR | 442 |
| | CDR2-b | FQNEAQ | 443 |
| | CDR3-b | CASSLVSAVGNIQYF | 1080 |
| | FWR1-b | DTGVSQDPRHKITKRGQNVTFRCDPI | 591 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GAGTRLSVL | 1081 |
| TCRA- | a-Variable | CAGAAGGAGGTGGAGCAGGATCCTGGACCACTCAGTGTTCCAGA | 1082 |
| 203 | nucleotide | GGGAGCCATTGTTTCTCTCAACTGCACTTACAGCAACAGTGCTTT | |
| | | TCAATACTTCATGTGGTACAGACAGTATTCCAGAAAAGGCCCTGA | |
| | | GTTGCTGATGTACACATACTCCAGTGGTAACAAAGAAGATGGAA | |
| | | GGTTTACAGCACAGGTCGATAAATCCAGCAAGTATATCTCCTTGT | |
| | | TCATCAGAGACTCACAGCCCAGTGATTCAGCCACCTACCTCTGTG | |
| | | CAATGAGCAATTATAACACCGACAAGCTCATCTTTGGGACTGGGA | |
| | | CCAGATTACAAGTCTTTCCAA | |
| | Variable-a | QKEVEQDPGPLSVPEGAIVSLNCTYSNSAFQYFMWYRQYSRKGPEL | 1083 |
| | | LMYTYSSGNKEDGRFTAQVDKSSKYISLFIRDSQPSDSATYLCAMSN | |
| | | YNTDKLIFGTGTRLQVFP | |
| | CDR1-a | NSAFQY | 673 |
| | CDR2-a | TYSSGN | 674 |
| | CDR3-a | CAMSNYNTDKLIF | 1084 |
| | FWR1-a | QKEVEQDPGPLSVPEGAIVSLNCTYS | 676 |
| | FWR2-a | FMWYRQYSRKGPELLMY | 677 |
| | FWR3-a | KEDGRFTAQVDKSSKYISLFIRDSQPSDSATYL | 678 |
| | FWR4-a | GTGTRLQVFP | 36 |
| TCRB- | b-Variable | GATGGTGGAATCACTCAGTCCCCAAAGTACCTGTTCAGAAAGGA | 1085 |
| 204 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTACCACCGGGGGGGACAGTCTTAGCAATCAGCC | |
| | | CCAGCATTTTGGTGATGGGACTCGACTCTCCATCCTAG | |
| | Variable-b | DGGITQSPKYLFRKEGQNVTLSCEQNLNHDAMYWYRQDPGQGLRLI | 1086 |
| | | YYSQIVNDFQKGDIAEGYSVSREKKESFPLTVTSAQKNPTAFYLCAS | |
| | | TTGGDSLSNQPQHFGDGTRLSIL | |
| | CDR1-b | LNHDA | 216 |
| | CDR2-b | SQIVND | 217 |
| | CDR3-b | CASTTGGDSLSNQPQHF | 1087 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GDGTRLSIL | 27 |
| TCRA- | a-Variable | CAGAAGGAGGTGGAGCAGAATTCTGGACCCCTCAGTGTTCCAGA | 1088 |
| 204 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTTCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAAACGCCCCAGGGATGCTGGTGGTACTAGCTATGGAAAG | |
| | | CTGACATTTGGACAAGGGACCATCTTGACTGTCCATCCAA | |
| | Variable-a | QKEVEQNSGPLSVPEGAIASLNCTYSDRGSQSFFWYRQYSGKSPELI | 1089 |
| | | MFIYSNGDKEDGRFTAQLNKASQYVSLLIRDSQPSDSATYLCAVKRP | |
| | | RDAGGTSYGKLTFGQGTILTVHP | |
| | CDR1-a | DRGSQS | 12 |
| | CDR2-a | IYSNGD | 13 |
| | CDR3-a | CAVKRPRDAGGTSYGKLTF | 1090 |
| | FWR1-a | QKEVEQNSGPLSVPEGAIASLNCTYS | 15 |
| | FWR2-a | FFWYRQYSGKSPELIMF | 16 |
| | FWR3-a | KEDGRFTAQLNKASQYVSLLIRDSQPSDSATYL | 17 |
| | FWR4-a | GQGTILTVHP | 76 |
| TCRB- | b-Variable | AATGCTGGTGTCACTCAGACCCCAAAATTCCAGGTCCTGAAGACA | 1091 |
| 205 | nucleotide | GGACAGAGCATGACACTGCAGTGTGCCCAGGATATGAACCATGA | |
| | | ATACATGTCCTGGTATCGACAAGACCCAGGCATGGGGCTGAGGC | |
| | | TGATTCATTACTCAGTTGGTGCTGGTATCACTGACCAAGGAGAAG | |
| | | TCCCCAATGGCTACAATGTCTCCAGATCAACCACAGAGGATTTCC | |
| | | CGCTCAGGCTGCTGTCGGCTGCTCCCTCCCAGACATCTGTGTACTT | |
| | | CTGTGCCAGCAGTTACTGGACAGGGACCTACGAGCAGTACTTCGG | |
| | | GCCGGGCACCAGGCTCACGGTCACAG | |
| | Variable-b | NAGVTQTPKFQVLKTGQSMTLQCAQDMNHEYMSWYRQDPGMGLR | 1092 |
| | | LIHYSVGAGITDQGEVPNGYNVSRSTTEDFPLRLLSAAPSQTSVYFCA | |
| | | SSYWTGTYEQYFGPGTRLTVT | |
| | CDR1-b | MNHEY | 252 |
| | CDR2-b | SVGAGI | 253 |
| | CDR3-b | CASSYWTGTYEQYF | 1093 |
| | FWR1-b | NAGVTQTPKFQVLKTGQSMTLQCAQD | 42 |
| | FWR2-b | MSWYRQDPGMGLRLIHY | 255 |
| | FWR3-b | TDQGEVPNGYNVSRSTTEDFPLRLLSAAPSQTSVYF | 256 |
| | FWR4-b | GPGTRLTVT | 85 |
| TCRA- | a-Variable | AGTCAACAGGGAGAAGAGGATCCTCAGGCCTTGAGCATCCAGGA | 1094 |
| 205 | nucleotide | GGGTGAAAATGCCACCATGAACTGCAGTTACAAAACTAGTATAA | |
| | | ACAATTTACAGTGGTATAGACAAAATTCAGGTAGAGGCCTTGTCC | |
| | | ACCTAATTTTAATACGTTCAAATGAAAGAGAGAAACACAGTGGA | |
| | | AGATTAAGAGTCACGCTTGACACTTCCAAGAAAAGCAGTTCCTTG | |
| | | TTGATCACGGCTTCCCGGGCAGCAGACACTGCTTCTTACTTCTGT | |
| | | GCTACGGTCTATTCAGGAGGAGGTGCTGACGGACTCACCTTTGGC | |
| | | AAAGGGACTCATCTAATCATCCAGCCCT | |
| | Variable-a | SQQGEEDPQALSIQEGENATMNCSYKTSINNLQWYRQNSGRGLVHL | 1095 |
| | | ILIRSNEREKHSGRLRVTLDTSKKSSSLLITASRAADTASYFCATVYS | |
| | | GGGADGLTFGKGTHLIIQP | |
| | CDR1-a | TSINN | 552 |
| | CDR2-a | IRSNERE | 553 |
| | CDR3-a | CATVYSGGGADGLTF | 1096 |
| | FWR1-a | SQQGEEDPQALSIQEGENATMNCSYK | 555 |
| | FWR2-a | LQWYRQNSGRGLVHLIL | 556 |
| | FWR3-a | KHSGRLRVTLDTSKKSSSLLITASRAADTASYF | 557 |
| | FWR4-a | GKGTHLIIQP | 363 |
| TCRB- | b-Variable | GATGGTGGAATCACTCAGTCCCCAAAGTACCTGTTCAGAAAGGA | 1097 |
| 206 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCAGTAGTAGCGGAACCTACAATGAGCAGTTCTTCGG | |
| | | GCCAGGGACACGGCTCACCGTGCTAG | |
| | Variable-b | DGGITQSPKYLFRKEGQNVTLSCEQNLNHDAMYWYRQDPGQGLRLI | 1098 |
| | | YYSQIVNDFQKGDIAEGYSVSREKKESFPLTVTSAQKNPTAFYLCAS | |
| | | SSGTYNEQFFGPGTRLTVL | |
| | CDR1-b | LNHDA | 216 |
| | CDR2-b | SQIVND | 217 |
| | CDR3-b | CASSSGTYNEQFF | 1099 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVL | 9 |
| TCRA- | a-Variable | GCCCAGAGAGTGACTCAGCCCGAGAAGCTCCTCTCTGTCTTTAAA | 1100 |
| 206 | nucleotide | GGGGCCCCAGTGGAGCTGAAGTGCAACTATTCCTATTCTGGGAGT | |
| | | CCTGAACTCTTCTGGTATGTCCAGTACTCCAGACAACGCCTCCAG | |
| | | TTACTCTTGAGACACATCTCTAGAGAGAGCATCAAAGGCTTCACT | |
| | | GCTGACCTTAACAAAGGCGAGACATCTTTCCACCTGAAGAAACC | |
| | | ATTTGCTCAAGAGGAAGACTCAGCCATGTATTACTGTGCTTCGCC | |
| | | GAGCAAAGCTGCAGGCAACAAGCTAACTTTTGGAGGAGGAACCA | |
| | | GGGTGCTAGTTAAACCAA | |
| | Variable-a | AQRVTQPEKLLSVFKGAPVELKCNYSYSGSPELFWYVQYSRQRLQL | 1101 |
| | | LLRHISRESIKGFTADLNKGETSFHLKKPFAQEEDSAMYYCASPSKA | |
| | | AGNKLTFGGGTRVLVKP | |
| | CDR1-a | YSGSPE | 70 |
| | CDR2-a | HISR | 71 |
| | CDR3-a | CASPSKAAGNKLTF | 1102 |
| | FWR1-a | AQRVTQPEKLLSVFKGAPVELKCNYS | 73 |
| | FWR2-a | LFWYVQYSRQRLQLLLR | 74 |
| | FWR3-a | ESIKGFTADLNKGETSFHLKKPFAQEEDSAMYY | 75 |
| | FWR4-a | GGGTRVLVKP | 1103 |
| TCRB- | b-Variable | GATACTGGAGTCTCCCAGAACCCCAGACACAAGATCACAAAGAG | 1104 |
| 207 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCAGCAGCTTAGAGGCCAGGGCCCCCTCTGGAAACA | |
| | | CCATATATTTTGGAGAGGGAAGTTGGCTCACTGTTGTAG | |
| | Variable-b | DTGVSQNPRHKITKRGQNVTFRCDPISEHNRLYWYRQTLGQGPEFLT | 1105 |
| | | YFQNEAQLEKSRLLSDRFSAERPKGSFSTLEIQRTEQGDSAMYLCASS | |
| | | LEARAPSGNTIYFGEGSWLTVV | |
| | CDR1-b | SEHNR | 442 |
| | CDR2-b | FQNEAQ | 443 |
| | CDR3-b | CASSLEARAPSGNTIYF | 1106 |
| | FWR1-b | DTGVSQNPRHKITKRGQNVTFRCDPI | 445 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GEGSWLTVV | 871 |
| TCRA- | a-Variable | GCTCAGAAGGTAACTCAAGCGCAGACTGAAATTTCTGTGGTGGA | 1107 |
| 207 | nucleotide | GAAGGAGGATGTGACCTTGGACTGTGTGTATGAAACCCGTGATA | |
| | | CTACTTATTACTTATTCTGGTACAAGCAACCACCAAGTGGAGAAT | |
| | | TGGTTTTCCTTATTCGTCGGAACTCTTTTGATGAGCAAAATGAAAT | |
| | | AAGTGGTCGGTATTCTTGGAACTTCCAGAAATCCACCAGTTCCTT | |
| | | CAACTTCACCATCACAGCCTCACAAGTCGTGGACTCAGCAGTATA | |
| | | CTTCTGTGCTCTATGGGAGGGTCAGGGCGGATCTGAAAAGCTGGT | |
| | | CTTTGGAAAGGGAACGAAACTGACAGTAAACCCAT | |
| | Variable-a | AQKVTQAQTEISVVEKEDVTLDCVYETRDTTYYLFWYKQPPSGELV | 1108 |
| | | FLIRRNSFDEQNEISGRYSWNFQKSTSSFNFTITASQVVDSAVYFCAL | |
| | | WEGQGGSEKLVFGKGTKLTVNP | |
| | CDR1-a | TRDTTYY | 48 |
| | CDR2-a | RNSFDEQN | 49 |
| | CDR3-a | CALWEGQGGSEKLVF | 1109 |
| | FWR1-a | AQKVTQAQTEISVVEKEDVTLDCVYE | 51 |
| | FWR2-a | LFWYKQPPSGEL VFLIR | 52 |
| | FWR3-a | EISGRYSWNFQKSTSSFNFTITASQVVDSAVYF | 53 |
| | FWR4-a | GKGTKLTVNP | 339 |
| TCRB- | b-Variable | GATACTGGAGTCTCCCAGAACCCCAGACACAAGATCACAAAGAG | 1110 |
| 208 | nucleotide | GGGACAGAATGTAACTTTCAGGTGTGATCCAATTTCTGAACACAA | |
| | | CCGCCTTTATTGGTACCGACAGACCCTGGGGCAGGGCCCAGAGTT | |
| | | TCTGACTTACTTCCAGAATGAAGCTCAACTAGAAAAATCAAGGCT | |
| | | GCTCAGTGATCGGTTCTCTGCAGAGAGGCCTAAGGGATCTTTCTC | |
| | | CACCTTGGAGATCCAGCGCACAGAGCAGGGGGACTCGGCCATGT | |
| | | ATCTCTGTGCCACCACACTCCCGGGGGACACTGAAGCTTTCTTTG | |
| | | GACAAGGCACCAGACTCACAGTTGTAG | |
| | Variable-b | DTGVSQNPRHKITKRGQNVTFRCDPISEHNRLYWYRQTLGQGPEFLT | 1111 |
| | | YFQNEAQLEKSRLLSDRFSAERPKGSFSTLEIQRTEQGDSAMYLCAT | |
| | | TLPGDTEAFFGQGTRLTVV | |
| | CDR1-b | SEHNR | 442 |
| | CDR2-b | FQNEAQ | 443 |
| | CDR3-b | CATTLPGDTEAFF | 1112 |
| | FWR1-b | DTGVSQNPRHKITKRGQNVTFRCDPI | 445 |
| | FWR2-b | LYWYRQTLGQGPEFLTY | 446 |
| | FWR3-b | LEKSRLLSDRFSAERPKGSFSTLEIQRTEQGDSAMYL | 447 |
| | FWR4-b | GQGTRLTVV | 246 |
| TCRA- | a-Variable | GCCCAGACAGTCACTCAGTCTCAACCAGAGATGTCTGTGCAGGA | 1113 |
| 208 | nucleotide | GGCAGAGACTGTGACCCTGAGTTGCACATATGACACCAGTGAGA | |
| | | GTAATTATTATTTGTTCTGGTACAAACAGCCTCCCAGCAGGCAGA | |
| | | TGATTCTCGTTATTCGCCAAGAAGCTTATAAGCAACAGAATGCAA | |
| | | CGGAGAATCGTTTCTCTGTGAACTTCCAGAAAGCAGCCAAATCCT | |
| | | TCAGTCTCAAGATCTCAGACTCACAGCTGGGGGACACTGCGATGT | |
| | | ATTTCTGTGCTTTCATGAAGGGGAGAGATGACAAGATCATCTTTG | |
| | | GAAAAGGGACACGACTTCATATTCTCCCCA | |
| | Variable-a | AQTVTQSQPEMSVQEAETVTLSCTYDTSESNYYLFWYKQPPSRQMI | 1114 |
| | | LVIRQEAYKQQNATENRFSVNFQKAAKSFSLKISDSQLGDTAMYFC | |
| | | AFMKGRDDKIIFGKGTRLHILP | |
| | CDR1-a | TSESNYY | 1115 |
| | CDR2-a | QEAYKQQN | 287 |
| | CDR3-a | CAFMKGRDDKIIF | 1116 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDTAMYF | 488 |
| | FWR4-a | GKGTRLHILP | 607 |
| TCRB- | b-Variable | GATGCTGGAGTTATCCAGTCACCCCGGCACGAGGTGACAGAGAT | 1117 |
| 209 | nucleotide | GGGACAAGAAGTGACTCTGAGATGTAAACCAATTTCAGGACACG | |
| | | ACTACCTTTTCTGGTACAGACAGACCATGATGCGGGGACTGGAGT | |
| | | TGCTCATTTACTTTAACAACAACGTTCCGATAGATGATTCAGGGA | |
| | | TGCCCGAGGATCGATTCTCAGCTAAGATGCCTAATGCATCATTCT | |
| | | CCACTCTGAAGATCCAGCCCTCAGAACCCAGGGACTCAGCTGTGT | |
| | | ACTTCTGTGCCAGCAGTTTGGACCGCCATCAGCCCCAGCATTTTG | |
| | | GTGATGGGACTCGACTCTCCATCCTAG | |
| | Variable-b | DAGVIQSPRHEVTEMGQEVTLRCKPISGHDYLFWYRQTMMRGLELL | 1118 |
| | | IYFNNNVPIDDSGMPEDRFSAKMPNASFSTLKIQPSEPRDSAVYFCAS | |
| | | SLDRHQPQHFGDGTRLSIL | |
| | CDR1-b | SGHDY | 468 |
| | CDR2-b | FNNNVP | 469 |
| | CDR3-b | CASSLDRHQPQHF | 1119 |
| | FWR1-b | DAGVIQSPRHEVTEMGQEVTLRCKPI | 471 |
| | FWR2-b | LFWYRQTMMRGLELLIY | 472 |
| | FWR3-b | IDDSGMPEDRFSAKMPNASFSTLKIQPSEPRDSAVYF | 473 |
| | FWR4-b | GDGTRLSIL | 27 |
| TCRA- | a-Variable | AGTCAACAGGGAGAAGAGGATCCTCAGGCCTTGAGCATCCAGGA | 1120 |
| 209 | nucleotide | GGGTGAAAATGCCACCATGAACTGCAGTTACAAAACTAGTATAA | |
| | | ACAATTTACAGTGGTATAGACAAAATTCAGGTAGAGGCCTTGTCC | |
| | | ACCTAATTTTAATACGTTCAAATGAAAGAGAGAAACACAGTGGA | |
| | | AGATTAAGAGTCACGCTTGACACTTCCAAGAAAAGCAGTTCCTTG | |
| | | TTGATCACGGCTTCCCGGGCAGCAGACACTGCTTCTTACTTCTGT | |
| | | GCTACGGCAAATAATGCAGGCAACATGCTCACCTTTGGAGGGGG | |
| | | AACAAGGTTAATGGTCAAACCCC | |
| | Variable-a | SQQGEEDPQALSIQEGENATMNCSYKTSINNLQWYRQNSGRGLVHL | 1121 |
| | | ILIRSNEREKHSGRLRVTLDTSKKSSSLLITASRAADTASYFCATANN | |
| | | AGNMLTFGGGTRLMVKP | |
| | CDR1-a | TSINN | 552 |
| | CDR2-a | IRSNERE | 553 |
| | CDR3-a | CATANNAGNMLTF | 1122 |
| | FWR1-a | SQQGEEDPQALSIQEGENATMNCSYK | 555 |
| | FWR2-a | LQWYRQNSGRGLVHLIL | 556 |
| | FWR3-a | KHSGRLRVTLDTSKKSSSLLITASRAADTASYF | 557 |
| | FWR4-a | GGGTRLMVKP | 141 |
| TCRB- | b-Variable | GACGCTGGAGTCACCCAAAGTCCCACACACCTGATCAAAACGAG | 1123 |
| 210 | nucleotide | AGGACAGCACGTGACTCTGAGATGCTCTCCTATCTCTGGGCACAA | |
| | | GAGTGTGTCCTGGTACCAACAGGTCCTGGGTCAGGGGCCCCAGTT | |
| | | TATCTTTCAGTATTATGAGAAAGAAGAGAGAGGAAGAGGAAACT | |
| | | TCCCTGATCGATTCTCAGCTCGCCAGTTCCCTAACTATAGCTCTGA | |
| | | GCTGAATGTGAACGCCTTGTTGCTGGGGGACTCGGCCCTGTATCT | |
| | | CTGTGCCAGCAGCTTGGAACTAGCGGGAGGGCGTGATACGCAGT | |
| | | ATTTTGGCCCAGGCACCCGGCTGACAGTGCTCG | |
| | Variable-b | DAGVTQSPTHLIKTRGQHVTLRCSPISGHKSVSWYQQVLGQGPQFIF | 1124 |
| | | QYYEKEERGRGNFPDRFSARQFPNYSSELNVNALLLGDSALYLCASS | |
| | | LELAGGRDTQYFGPGTRLTVL | |
| | CDR1-b | SGHKS | 1125 |
| | CDR2-b | YYEKEE | 1126 |
| | CDR3-b | CASSLELAGGRDTQYF | 1127 |
| | FWR1-b | DAGVTQSPTHLIKTRGQHVTLRCSPI | 1128 |
| | FWR2-b | VSWYQQVLGQGPQFIFQ | 1129 |
| | FWR3-b | RGRGNFPDRFSARQFPNYSSELNVNALLLGDSALYL | 1130 |
| | FWR4-b | GPGTRLTVL | 9 |
| TCRA- | a-Variable | GCCCAGTCAGTGACCCAGCCTGACATCCACATCACTGTCTCTGAA | 1131 |
| 210 | nucleotide | GGAGCCTCACTGGAGTTGAGATGTAACTATTCCTATGGGGCAACA | |
| | | CCTTATCTCTTCTGGTATGTCCAGTCCCCCGGCCAAGGCCTCCAGC | |
| | | TGCTCCTGAAGTACTTTTCAGGAGACACTCTGGTTCAAGGCATTA | |
| | | AAGGCTTTGAGGCTGAATTTAAGAGGAGTCAATCTTCCTTCAACC | |
| | | TGAGGAAACCCTCTGTGCATTGGAGTGATGCTGCTGAGTACTTCT | |
| | | GTGCTGTGGGTGCCGGAACCGGCACTGCCAGTAAACTCACCTTTG | |
| | | GGACTGGAACAAGACTTCAGGTCACGCTCG | |
| | Variable-a | AQSVTQPDIHITVSEGASLELRCNYSYGATPYLFWYVQSPGQGLQLL | 1132 |
| | | LKYFSGDTLVQGIKGFEAEFKRSQSSFNLRKPSVHWSDAAEYFCAVG | |
| | | AGTGTASKLTFGTGTRLQVTL | |
| | CDR1-a | YGATPY | 886 |
| | CDR2-a | YFSGDTLV | 887 |
| | CDR3-a | CAVGAGTGTASKLTF | 1133 |
| | FWR1-a | AQSVTQPDIHITVSEGASLELRCNYS | 889 |
| | FWR2-a | LFWYVQSPGQGLQLLLK | 890 |
| | FWR3-a | QGIKGFEAEFKRSQSSFNLRKPSVHWSDAAEYF | 891 |
| | FWR4-a | GTGTRLQVTL | 1134 |
| TCRB- | b-Variable | GATTCTGGAGTCACACAAACCCCAAAGCACCTGATCACAGCAAC | 1135 |
| 211 | nucleotide | TGGACAGCGAGTGACGCTGAGATGCTCCCCTAGGTCTGGAGACCT | |
| | | CTCTGTGTACTGGTACCAACAGAGCCTGGACCAGGGCCTCCAGTT | |
| | | CCTCATTCAGTATTATAATGGAGAAGAGAGAGCAAAAGGAAACA | |
| | | TTCTTGAACGATTCTCCGCACAACAGTTCCCTGACTTGCACTCTGA | |
| | | ACTAAACCTGAGCTCTCTGGAGCTGGGGGACTCAGCTTTGTATTT | |
| | | CTGTGCCAGCAGCGAGGGGACAGTCCTTGACGAGCAGTACTTCG | |
| | | GGCCGGGCACCAGGCTCACGGTCACAG | |
| | Variable-b | DSGVTQTPKHLITATGQRVTLRCSPRSGDLSVYWYQQSLDQGLQFLI | 1136 |
| | | QYYNGEERAKGNILERFSAQQFPDLHSELNLSSLELGDSALYFCASSE | |
| | | GTVLDEQYFGPGTRLTVT | |
| | CDR1-b | SGDLS | 383 |
| | CDR2-b | YYNGEE | 384 |
| | CDR3-b | CASSEGTVLDEQYF | 1137 |
| | FWR1-b | DSGVTQTPKHLITATGQRVTLRCSPR | 386 |
| | FWR2-b | VYWYQQSLDQGLQFLIQ | 387 |
| | FWR3-b | RAKGNILERFSAQQFPDLHSELNLSSLELGDSALYF | 388 |
| | FWR4-b | GPGTRLTVT | 85 |
| TCRA- | a-Variable | GCTCAGAAGGTAACTCAAGCGCAGACTGAAATTTCTGTGGTGGA | 1138 |
| 211 | nucleotide | GAAGGAGGATGTGACCTTGGACTGTGTGTATGAAACCCGTGATA | |
| | | CTACTTATTACTTATTCTGGTACAAGCAACCACCAAGTGGAGAAT | |
| | | TGGTTTTCCTTATTCGTCGGAACTCTTTTGATGAGCAAAATGAAAT | |
| | | AAGTGGTCGGTATTCTTGGAACTTCCAGAAATCCACCAGTTCCTT | |
| | | CAACTTCACCATCACAGCCTCACAAGTCGTGGACTCAGCAGTATA | |
| | | CTTCTGTGCTCTGAGTGAAGGATCTAACTTTGGAAATGAGAAATT | |
| | | AACCTTTGGGACTGGAACAAGACTCACCATCATACCCA | |
| | Variable-a | AQKVTQAQTEISVVEKEDVTLDCVYETRDTTYYLFWYKQPPSGELV | 1139 |
| | | FLIRRNSFDEQNEISGRYSWNFQKSTSSFNFTITASQVVDSAVYFCAL | |
| | | SEGSNFGNEKLTFGTGTRLTIIP | |
| | CDR1-a | TRDTTYY | 48 |
| | CDR2-a | RNSFDEQN | 49 |
| | CDR3-a | CALSEGSNFGNEKLTF | 1140 |
| | FWR1-a | AQKVTQAQTEISVVEKEDVTLDCVYE | 51 |
| | FWR2-a | LFWYKQPPSGEL VFLIR | 52 |
| | FWR3-a | EISGRYSWNFQKSTSSFNFTITASQVVDSAVYF | 53 |
| | FWR4-a | GTGTRLTIIP | 292 |
| TCRB- | b-Variable | GAAGCCCAAGTGACCCAGAACCCAAGATACCTCATCACAGTGAC | 1141 |
| 212 | nucleotide | TGGAAAGAAGTTAACAGTGACTTGTTCTCAGAATATGAACCATGA | |
| | | GTATATGTCCTGGTATCGACAAGACCCAGGGCTGGGCTTAAGGCA | |
| | | GATCTACTATTCAATGAATGTTGAGGTGACTGATAAGGGAGATGT | |
| | | TCCTGAAGGGTACAAAGTCTCTCGAAAAGAGAAGAGGAATTTCC | |
| | | CCCTGATCCTGGAGTCGCCCAGCCCCAACCAGACCTCTCTGTACT | |
| | | TCTGTGCCAGCAGTTTGGGGTCGTCTACGCAGTATTTTGGCCCAG | |
| | | GCACCCGGCTGACAGTGCTCG | |
| | Variable-b | EAQVTQNPRYLITVTGKKLTVTCSQNMNHEYMSWYRQDPGLGLRQ | 1142 |
| | | IYYSMNVEVTDKGDVPEGYKVSRKEKRNFPLILESPSPNQTSLYFCA | |
| | | SSLGSSTQYFGPGTRLTVL | |
| | CDR1-b | MNHEY | 252 |
| | CDR2-b | SMNVEV | 366 |
| | CDR3-b | CASSLGSSTQYF | 1143 |
| | FWR1-b | EAQVTQNPRYLITVTGKKLTVTCSQN | 368 |
| | FWR2-b | MSWYRQDPGLGLRQIYY | 369 |
| | FWR3-b | TDKGDVPEGYKVSRKEKRNFPLILESPSPNQTSLYF | 370 |
| | FWR4-b | GPGTRLTVL | 9 |
| TCRA- | a-Variable | AAACAGGAGGTGACGCAGATTCCTGCAGCTCTGAGTGTCCCAGA | 1144 |
| 212 | nucleotide | AGGAGAAAACTTGGTTCTCAACTGCAGTTTCACTGATAGCGCTAT | |
| | | TTACAACCTCCAGTGGTTTAGGCAGGACCCTGGGAAAGGTCTCAC | |
| | | ATCTCTGTTGCTTATTCAGTCAAGTCAGAGAGAGCAAACAAGTGG | |
| | | AAGACTTAATGCCTCGCTGGATAAATCATCAGGACGTAGTACTTT | |
| | | ATACATTGCAGCTTCTCAGCCTGGTGACTCAGCCACCTACCTCTG | |
| | | TGCTGCGGGGGGGAATTATGGAGGAAGCCAAGGAAATCTCATCT | |
| | | TTGGAAAAGGCACTAAACTCTCTGTTAAACCAA | |
| | Variable-a | KQEVTQIPAALSVPEGENLVLNCSFTDSAIYNLQWFRQDPGKGLTSL | 1145 |
| | | LLIQSSQREQTSGRLNASLDKSSGRSTLYIAASQPGDSATYLCAAGG | |
| | | NYGGSQGNLIFGKGTKLSVKP | |
| | CDR1-a | DSAIYN | 118 |
| | CDR2-a | IQSSQRE | 119 |
| | CDR3-a | CAAGGNYGGSQGNLIF | 1146 |
| | FWR1-a | KQEVTQIPAALSVPEGENLVLNCSFT | 121 |
| | FWR2-a | LQWFRQDPGKGLTSLLL | 122 |
| | FWR3-a | QTSGRLNASLDKSSGRSTLYIAASQPGDSATYL | 123 |
| | FWR4-a | GKGTKLSVKP | 124 |
| TCRB- | b-Variable | AATGCTGGTGTCACTCAGACCCCAAAATTCCGGGTCCTGAAGACA | 1262 |
| 213 | nucleotide | GGACAGAGCATGACACTGCTGTGTGCCCAGGATATGAACCATGA | |
| | | ATACATGTACTGGTATCGACAAGACCCAGGCATGGGGCTGAGGC | |
| | | TGATTCATTACTCAGTTGGTGAGGGTACAACTGCCAAAGGAGAG | |
| | | GTCCCTGATGGCTACAATGTCTCCAGATTAAAAAAACAGAATTTC | |
| | | CTGCTGGGGTTGGAGTCGGCTGCTCCCTCCCAAACATCTGTGTAC | |
| | | TTCTGTGCCAGCAGCTTCATCGGTTGGGGCTTAGCACCCCGGGGG | |
| | | GCCCAGTACTTCGGGCCAGGCACGCGGCTCCTGGTGCTCG | |
| | Variable-b | NAGVTQTPKFRVLKTGQSMTLLCAQDMNHEYMYWYRQDPGMGLR | 1263 |
| | | LIHYSVGEGTTAKGEVPDGYNVSRLKKQNFLLGLESAAPSQTSVYFC | |
| | | ASSFIGWGLAPRGAQYFGPGTRLLVL | |
| | CDR1-b | MNHEY | 252 |
| | CDR2-b | SVGEGT | 1046 |
| | CDR3-b | CASSFIGWGLAPRGAQYF | 1264 |
| | FWR1-b | NAGVTQTPKFRVLKTGQSMTLLCAQD | 1048 |
| | FWR2-b | MYWYRQDPGMGLRLIHY | 1049 |
| | FWR3-b | TAKGEVPDGYNVSRLKKQNFLLGLESAAPSQTSVYF | 1050 |
| | FWR4-b | GPGTRLLVL | 67 |
| TCRA- | a-Variable | GCTCAGTCAGTGGCTCAGCCGGAAGATCAGGTCAACGTTGCTGA | 1265 |
| 213 | nucleotide | AGGGAATCCTCTGACTGTGAAATGCACCTATTCAGTCTCTGGAAA | |
| | | CCCTTATCTTTTTTGGTATGTTCAATACCCCAACCGAGGCCTCCAG | |
| | | TTCCTTCTGAAATACATCACAGGGGATAACCTGGTTAAAGGCAGC | |
| | | TATGGCTTTGAAGCTGAATTTAACAAGAGCCAAACCTCCTTCCAC | |
| | | CTGAAGAAACCATCTGCCCTTGTGAGCGACTCCGCTTTGTACTTC | |
| | | TGTGCTGTGAGATTCGACAGAGGCTCAACCCTGGGGAGGCTATAC | |
| | | TTTGGAAGAGGAACTCAGTTGACTGTCTGGCCTG | |
| | Variable-a | AQSVAQPEDQVNVAEGNPLTVKCTYSVSGNPYLFWYVQYPNRGLQ | 1266 |
| | | FLLKYITGDNLVKGSYGFEAEFNKSQTSFHLKKPSALVSDSALYFCA | |
| | | VRFDRGSTLGRLYFGRGTQLTVWP | |
| | CDR1-a | VSGNPY | 427 |
| | CDR2-a | YITGDNLV | 428 |
| | CDR3-a | CAVRFDRGSTLGRLYF | 1267 |
| | FWR1-a | AQSVAQPEDQVNVAEGNPLTVKCTYS | 430 |
| | FWR2-a | LFWYVQYPNRGLQFLLK | 431 |
| | FWR3-a | KGSYGFEAEFNKSQTSFHLKKPSALVSDSALYF | 432 |
| | FWR4-a | GRGTQLTVWP | 190 |
| TCRB- | b-Variable | GACACTGAAGTTACCCAGACACCAAAACACCTGGTCATGGGAAT | 1268 |
| 214 | nucleotide | GACAAATAAGAAGTCTTTGAAATGTGAACAACATATGGGGCACA | |
| | | GGGCTATGTATTGGTACAAGCAGAAAGCTAAGAAGCCACCGGAG | |
| | | CTCATGTTTGTCTACAGCTATGAGAAACTCTCTATAAATGAAAGT | |
| | | GTGCCAAGTCGCTTCTCACCTGAATGCCCCAACAGCTCTCTCTTA | |
| | | AACCTTCACCTACACGCCCTGCAGCCAGAAGACTCAGCCCTGTAT | |
| | | CTCTGCGCCAGCAGCCTGAGAGAAAAACTGTTTTTTGGCAGTGGA | |
| | | ACCCAGCTCTCTGTCTTGG | |
| | Variable-b | DTEVTQTPKHLVMGMTNKKSLKCEQHMGHRAMYWYKQKAKKPP | 1269 |
| | | ELMFVYSYEKLSINESVPSRESPECPNSSLLNLHLHALQPEDSALYLC | |
| | | ASSLREKLFFGSGTQLSVL | |
| | CDR1-b | MGHRA | 639 |
| | CDR2-b | YSYEKL | 640 |
| | CDR3-b | CASSLREKLFF | 1270 |
| | FWR1-b | DTEVTQTPKHLVMGMTNKKSLKCEQH | 642 |
| | FWR2-b | MYWYKQKAKKPPELMFV | 643 |
| | FWR3-b | SINESVPSRFSPECPNSSLLNLHLHALQPEDSALYL | 644 |
| | FWR4-b | GSGTQLSVL | 45 |
| TCRA- | a-Variable | GGAGAGGATGTGGAGCAGAGTCTTTTCCTGAGTGTCCGAGAGGG | 1271 |
| 214 | nucleotide | AGACAGCTCCGTTATAAACTGCACTTACACAGACAGCTCCTCCAC | |
| | | CTACTTATACTGGTATAAGCAAGAACCTGGAGCAGGTCTCCAGTT | |
| | | GCTGACGTATATTTTTTCAAATATGGACATGAAACAAGACCAAAG | |
| | | ACTCACTGTTCTATTGAATAAAAAGGATAAACATCTGTCTCTGCG | |
| | | CATTGCAGACACCCAGACTGGGGACTCAGCTATCTACTTCTGTGC | |
| | | AGAAAATGCTGGCAACAACCGTAAGCTGATTTGGGGATTGGGAA | |
| | | CAAGCCTGGCAGTAAATCCGA | |
| | Variable-a | GEDVEQSLFLSVREGDSSVINCTYTDSSSTYLYWYKQEPGAGLQLLT | 1272 |
| | | YIFSNMDMKQDQRLTVLLNKKDKHLSLRIADTQTGDSAIYFCAENA | |
| | | GNNRKLIWGLGTSLAVNP | |
| | CDR1-a | DSSSTY | 314 |
| | CDR2-a | IFSNMDM | 315 |
| | CDR3-a | CAENAGNNRKLIW | 1273 |
| | FWR1-a | GEDVEQSLFLSVREGDSSVINCTYT | 317 |
| | FWR2-a | LYWYKQEPGAGLQLLTY | 318 |
| | FWR3-a | KQDQRLTVLLNKKDKHLSLRIADTQTGDSAIYF | 319 |
| | FWR4-a | GLGTSLAVNP | 242 |
| TCRB- | b-Variable | GATGCTGGAGTTATCCAGTCACCCCGGCACGAGGTGACAGAGAT | 1274 |
| 215 | nucleotide | GGGACAAGAAGTGACTCTGAGATGTAAACCAATTTCAGGACACG | |
| | | ACTACCTTTTCTGGTACAGACAGACCATGATGCGGGGACTGGAGT | |
| | | TGCTCATTTACTTTAACAACAACGTTCCGATAGATGATTCAGGGA | |
| | | TGCCCGAGGATCGATTCTCAGCTAAGATGCCTAATGCATCATTCT | |
| | | CCACTCTGAAGATCCAGCCCTCAGAACCCAGGGACTCAGCTGTGT | |
| | | ACTTCTGTGCCAGCAGTCGGGGACAGGGTGTCTCCTACGAGCAGT | |
| | | ACTTCGGGCCGGGCACCAGGCTCACGGTCACAG | |
| | Variable-b | DAGVIQSPRHEVTEMGQEVTLRCKPISGHDYLFWYRQTMMRGLELL | 1275 |
| | | IYFNNNVPIDDSGMPEDRFSAKMPNASFSTLKIQPSEPRDSAVYFCAS | |
| | | SRGQGVSYEQYFGPGTRLTVT | |
| | CDR1-b | SGHDY | 468 |
| | CDR2-b | FNNNVP | 469 |
| | CDR3-b | CASSRGQGVSYEQYF | 1276 |
| | FWR1-b | DAGVIQSPRHEVTEMGQEVTLRCKPI | 471 |
| | FWR2-b | LFWYRQTMMRGLELLIY | 472 |
| | FWR3-b | IDDSGMPEDRFSAKMPNASFSTLKIQPSEPRDSAVYF | 473 |
| | FWR4-b | GPGTRLTVT | 85 |
| TCRA- | a-Variable | CGGAAGGAGGTGGAGCAGGATCCTGGACCCTTCAATGTTCCAGA | 1277 |
| 215 | nucleotide | GGGAGCCACTGTCGCTTTCAACTGTACTTACAGCAACAGTGCTTC | |
| | | TCAGTCTTTCTTCTGGTACAGACAGGATTGCAGGAAAGAACCTAA | |
| | | GTTGCTGATGTCCGTATACTCCAGTGGTAATGAAGATGGAAGGTT | |
| | | TACAGCACAGCTCAATAGAGCCAGCCAGTATATTTCCCTGCTCAT | |
| | | CAGAGACTCCAAGCTCAGTGATTCAGCCACCTACCTCTGTGTGGT | |
| | | GAGGGCTAACTTTGGAAATGAGAAATTAACCTTTGGGACTGGAA | |
| | | CAAGACTCACCATCATACCCA | |
| | Variable-a | RKEVEQDPGPFNVPEGATVAFNCTYSNSASQSFFWYRQDCRKEPKL | 1278 |
| | | LMSVYSSGNEDGRFTAQLNRASQYISLLIRDSKLSDSATYLCVVRAN | |
| | | FGNEKLTFGTGTRLTIIP | |
| | CDR1-a | NSASQS | 224 |
| | CDR2-a | VYSSG | 225 |
| | CDR3-a | CVVRANFGNEKLTF | 1279 |
| | FWR1-a | RKEVEQDPGPFNVPEGATVAFNCTYS | 227 |
| | FWR2-a | FFWYRQDCRKEPKLLMS | 228 |
| | FWR3-a | NEDGRFTAQLNRASQYISLLIRDSKLSDSATYL | 229 |
| | FWR4-a | GTGTRLTIIP | 292 |
| TCRB- | b-Variable | GGTGCTGTCGTCTCTCAACATCCGAGCTGGGTTATCTGTAAGAGT | 1280 |
| 216 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGTGGACAGACAGATACGCAGTATT | |
| | | TTGGCCCAGGCACCCGGCTGACAGTGCTCG | |
| | Variable-b | GAVVSQHPSWVICKSGTSVKIECRSLDFQATTMFWYRQFPKQSLML | 1281 |
| | | MATSNEGSKATYEQGVEKDKFLINHASLTLSTLTVTSAHPEDSSFYIC | |
| | | SASGQTDTQYFGPGTRLTVL | |
| | CDR1-b | DFQATT | 79 |
| | CDR2-b | SNEGSKA | 80 |
| | CDR3-b | CSASGQTDTQYF | 1282 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVL | 9 |
| TCRA- | a-Variable | CAGAAGGAGGTGGAGCAGGATCCTGGACCACTCAGTGTTCCAGA | 1283 |
| 216 | nucleotide | GGGAGCCATTGTTTCTCTCAACTGCACTTACAGCAACAGTGCTTT | |
| | | TCAATACTTCATGTGGTACAGACAGTATTCCAGAAAAGGCCCTGA | |
| | | GTTGCTGATGTACACATACTCCAGTGGTAACAAAGAAGATGGAA | |
| | | GGTTTACAGCACAGGTCGATAAATCCAGCAAGTATATCTCCTTGT | |
| | | TCATCAGAGACTCACAGCCCAGTGATTCAGCCACCTACCTCTGTG | |
| | | CAATGAGGGCTTATACTGGAGGCTTCAAAACTATCTTTGGAGCAG | |
| | | GAACAAGACTATTTGTTAAAGCAA | |
| | Variable-a | QKEVEQDPGPLSVPEGAIVSLNCTYSNSAFQYFMWYRQYSRKGPEL | 1284 |
| | | LMYTYSSGNKEDGRFTAQVDKSSKYISLFIRDSQPSDSATYLCAMRA | |
| | | YTGGFKTIFGAGTRLFVKA | |
| | CDR1-a | NSAFQY | 673 |
| | CDR2-a | TYSSGN | 674 |
| | CDR3-a | CAMRAYTGGFKTIF | 1285 |
| | FWR1-a | QKEVEQDPGPLSVPEGAIVSLNCTYS | 676 |
| | FWR2-a | FMWYRQYSRKGPELLMY | 677 |
| | FWR3-a | KEDGRFTAQVDKSSKYISLFIRDSQPSDSATYL | 678 |
| | FWR4-a | GAGTRLFVKA | 112 |
| TCRB- | b-Variable | GGTGCTGTCGTCTCTCAACATCCGAGCTGGGTTATCTGTAAGAGT | 1286 |
| 217 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGGCCGAGGGGCAACACTGAAGCTT | |
| | | TCTTTGGACAAGGCACCAGACTCACAGTTGTAG | |
| | Variable-b | GAVVSQHPSWVICKSGTSVKIECRSLDFQATTMFWYRQFPKQSLML | 1287 |
| | | MATSNEGSKATYEQGVEKDKFLINHASLTLSTLTVTSAHPEDSSFYIC | |
| | | SARPRGNTEAFFGQGTRLTVV | |
| | CDR1-b | DFQATT | 79 |
| | CDR2-b | SNEGSKA | 80 |
| | CDR3-b | CSARPRGNTEAFF | 1288 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GQGTRLTVV | 246 |
| TCRA- | a-Variable | GGAATACAAGTGGAGCAGAGTCCTCCAGACCTGATTCTCCAGGA | 1289 |
| 217 | nucleotide | GGGAGCCAATTCCACGCTGCGGTGCAATTTTTCTGACTCTGTGAA | |
| | | CAATTTGCAGTGGTTTCATCAAAACCCTTGGGGACAGCTCATCAA | |
| | | CCTGTTTTACATTCCCTCAGGGACAAAACAGAATGGAAGATTAAG | |
| | | CGCCACGACTGTCGCTACGGAACGCTACAGCTTATTGTACATTTC | |
| | | CTCTTCCCAGACCACAGACTCAGGCGTTTATTTCTGTGCTGTGCG | |
| | | GCTGAATTATGGTGGTGCTACAAACAAGCTCATCTTTGGAACTGG | |
| | | CACTCTGCTTGCTGTCCAGCCAA | |
| | Variable-a | GIQVEQSPPDLILQEGANSTLRCNFSDSVNNLQWFHQNPWGQLINLF | 1290 |
| | | YIPSGTKQNGRLSATTVATERYSLLYISSSQTTDSGVYFCAVRLNYG | |
| | | GATNKLIFGTGTLLAVQP | |
| | CDR1-a | DSVNN | 506 |
| | CDR2-a | IPSGT | 507 |
| | CDR3-a | CAVRLNYGGATNKLIF | 1291 |
| | FWR1-a | GIQVEQSPPDLILQEGANSTLRCNFS | 509 |
| | FWR2-a | LQWFHQNPWGQLINLFY | 510 |
| | FWR3-a | KQNGRLSATTVATERYSLLYISSSQTTDSGVYF | 511 |
| | FWR4-a | GTGTLLAVQP | 480 |
| TCRB- | b-Variable | GGTGCTGTCGTCTCTCAACATCCGAGCTGGGTTATCTGTAAGAGT | 1292 |
| 218 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGACCGCCTCAGGGGGACACTGAAG | |
| | | CTTTCTTTGGACAAGGCACCAGACTCACAGTTGTAG | |
| | Variable-b | GAVVSQHPSWVICKSGTSVKIECRSLDFQATTMFWYRQFPKQSLML | 1293 |
| | | MATSNEGSKATYEQGVEKDKFLINHASLTLSTLTVTSAHPEDSSFYIC | |
| | | SARPPQGDTEAFFGQGTRLTVV | |
| | CDR1-b | DFQATT | 79 |
| | CDR2-b | SNEGSKA | 80 |
| | CDR3-b | CSARPPQGDTEAFF | 1294 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GQGTRLTVV | 246 |
| TCRA- | a-Variable | GGACAAAACATTGACCAGCCCACTGAGATGACAGCTACGGAAGG | 1295 |
| 218 | nucleotide | TGCCATTGTCCAGATCAACTGCACGTACCAGACATCTGGGTTCAA | |
| | | CGGGCTGTTCTGGTACCAGCAACATGCTGGCGAAGCACCCACATT | |
| | | TCTGTCTTACAATGTTCTGGATGGTTTGGAGGAGAAAGGTCGTTT | |
| | | TTCTTCATTCCTTAGTCGGTCTAAAGGGTACAGTTACCTCCTTTTG | |
| | | AAGGAGCTCCAGATGAAAGACTCTGCCTCTTACCTCTGTGCTGTT | |
| | | ATGGATAGCAACTATCAGTTAATCTGGGGCGCTGGGACCAAGCT | |
| | | AATTATAAAGCCAG | |
| | Variable-a | GQNIDQPTEMTATEGAIVQINCTYQTSGFNGLFWYQQHAGEAPTFLS | 1296 |
| | | YNVLDGLEEKGRFSSFLSRSKGYSYLLLKELQMKDSASYLCAVMDS | |
| | | NYQLIWGAGTKLIIKP | |
| | CDR1-a | TSGFNG | 450 |
| | CDR2-a | NVLDGL | 451 |
| | CDR3-a | CAVMDSNYQLIW | 1297 |
| | FWR1-a | GQNIDQPTEMTATEGAIVQINCTYQ | 453 |
| | FWR2-a | LFWYQQHAGEAPTFLSY | 454 |
| | FWR3-a | EEKGRFSSFLSRSKGYSYLLLKELQMKDSASYL | 455 |
| | FWR4-a | GAGTKLIIKP | 356 |
| TCRB- | b-Variable | GATGCTGATGTTACCCAGACCCCAAGGAATAGGATCACAAAGAC | 1298 |
| 219 | nucleotide | AGGAAAGAGGATTATGCTGGAATGTTCTCAGACTAAGGGTCATG | |
| | | ATAGAATGTACTGGTATCGACAAGACCCAGGACTGGGCCTACGG | |
| | | TTGATCTATTACTCCTTTGATGTCAAAGATATAAACAAAGGAGAG | |
| | | ATCTCTGATGGATACAGTGTCTCTCGACAGGCACAGGCTAAATTC | |
| | | TCCCTGTCCCTAGAGTCTGCCATCCCCAACCAGACAGCTCTTTACT | |
| | | TCTGTGCCACTCAGGGTAGCAATCAGCCCCAGCATTTTGGTGATG | |
| | | GGACTCGACTCTCCATCCTAG | |
| | Variable-b | DADVTQTPRNRITKTGKRIMLECSQTKGHDRMYWYRQDPGLGLRLI | 1299 |
| | | YYSFDVKDINKGEISDGYSVSRQAQAKFSLSLESAIPNQTALYFCATQ | |
| | | GSNQPQHFGDGTRLSIL | |
| | CDR1-b | KGHDR | 21 |
| | CDR2-b | SFDVKD | 22 |
| | CDR3-b | CATQGSNQPQHF | 1300 |
| | FWR1-b | DADVTQTPRNRITKTGKRIMLECSQT | 24 |
| | FWR2-b | MYWYRQDPGLGLRLIYY | 25 |
| | FWR3-b | INKGEISDGYSVSRQAQAKFSLSLESAIPNQTALYF | 26 |
| | FWR4-b | GDGTRLSIL | 27 |
| TCRA- | a-Variable | CAGAAGGAGGTGGAGCAGAATTCTGGACCCCTCAGTGTTCCAGA | 1301 |
| 219 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTCCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAACGCTGCAGGCAACAAGCTAACTTTTGGAGGAGGAACC | |
| | | AGGGTGCTAGTTAAACCAA | |
| | Variable-a | QKEVEQNSGPLSVPEGAIASLNCTYSDRGSQSFFWYRQYSGKSPELI | 1302 |
| | | MSIYSNGDKEDGRFTAQLNKASQYVSLLIRDSQPSDSATYLCAVNA | |
| | | AGNKLTFGGGTRVLVKP | |
| | CDR1-a | DRGSQS | 12 |
| | CDR2-a | IYSNGD | 13 |
| | CDR3-a | CAVNAAGNKLTF | 1303 |
| | FWR1-a | QKEVEQNSGPLSVPEGAIASLNCTYS | 15 |
| | FWR2-a | FFWYRQYSGKSPELIMS | 1304 |
| | FWR3-a | KEDGRFTAQLNKASQYVSLLIRDSQPSDSATYL | 17 |
| | FWR4-a | GGGTRVLVKP | 1103 |
| TCRB- | b-Variable | GATGGTGGAATCACTCAGTCCCCAAAGTACCTGTTCAGAAAGGA | 1305 |
| 220 | nucleotide | AGGACAGAATGTGACCCTGAGTTGTGAACAGAATTTGAACCACG | |
| | | ATGCCATGTACTGGTACCGACAGGACCCAGGGCAAGGGCTGAGA | |
| | | TTGATCTACTACTCACAGATAGTAAATGACTTTCAGAAAGGAGAT | |
| | | ATAGCTGAAGGGTACAGCGTCTCTCGGGAGAAGAAGGAATCCTT | |
| | | TCCTCTCACTGTGACATCGGCCCAAAAGAACCCGACAGCTTTCTA | |
| | | TCTCTGTGCCACTTCGGTTTATTACAATGAGCAGTTCTTCGGGCCA | |
| | | GGGACACGGCTCACCGTGCTAG | |
| | Variable-b | DGGITQSPKYLFRKEGQNVTLSCEQNLNHDAMYWYRQDPGQGLRLI | 1306 |
| | | YYSQIVNDFQKGDIAEGYSVSREKKESFPLTVTSAQKNPTAFYLCAT | |
| | | SVYYNEQFFGPGTRLTVL | |
| | CDR1-b | LNHDA | 216 |
| | CDR2-b | SQIVND | 217 |
| | CDR3-b | CATSVYYNEQFF | 1307 |
| | FWR1-b | DGGITQSPKYLFRKEGQNVTLSCEQN | 219 |
| | FWR2-b | MYWYRQDPGQGLRLIYY | 220 |
| | FWR3-b | FQKGDIAEGYSVSREKKESFPLTVTSAQKNPTAFYL | 221 |
| | FWR4-b | GPGTRLTVL | 9 |
| TCRA- | a-Variable | CAGAAGGAGGTGGAGCAGGATCCTGGACCACTCAGTGTTCCAGA | 1308 |
| 220 | nucleotide | GGGAGCCATTGTTTCTCTCAACTGCACTTACAGCAACAGTGCTTT | |
| | | TCAATACTTCATGTGGTACAGACAGTATTCCAGAAAAGGCCCTGA | |
| | | GTTGCTGATGTACACATACTCCAGTGGTAACAAAGAAGATGGAA | |
| | | GGTTTACAGCACAGGTCGATAAATCCAGCAAGTATATCTCCTTGT | |
| | | TCATCAGAGACTCACAGCCCAGTGATTCAGCCACCTACCTCTGTG | |
| | | CAATGACCGATAACACCGACAAGCTCATCTTTGGGACTGGGACC | |
| | | AGATTACAAGTCTTTCCAA | |
| | Variable-a | QKEVEQDPGPLSVPEGAIVSLNCTYSNSAFQYFMWYRQYSRKGPEL | 1309 |
| | | LMYTYSSGNKEDGRFTAQVDKSSKYISLFIRDSQPSDSATYLCAMTD | |
| | | NTDKLIFGTGTRLQVFP | |
| | CDR1-a | NSAFQY | 673 |
| | CDR2-a | TYSSGN | 674 |
| | CDR3-a | CAMTDNTDKLIF | 1310 |
| | FWR1-a | QKEVEQDPGPLSVPEGAIVSLNCTYS | 676 |
| | FWR2-a | FMWYRQYSRKGPELLMY | 677 |
| | FWR3-a | KEDGRFTAQVDKSSKYISLFIRDSQPSDSATYL | 678 |
| | FWR4-a | GTGTRLQVFP | 36 |
| TCRB- | b-Variable | GGTGCTGTCGTCTCTCAACATCCGAGCTGGGTTATCTGTAAGAGT | 1311 |
| 221 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTCGACTGTGGGGGCTAACCTACGAGC | |
| | | AGTACTTCGGGCCGGGCACCAGGCTCACGGTCACAG | |
| | Variable-b | GAVVSQHPSWVICKSGTSVKIECRSLDFQATTMFWYRQFPKQSLML | 1312 |
| | | MATSNEGSKATYEQGVEKDKFLINHASLTLSTLTVTSAHPEDSSFYIC | |
| | | SARLWGLTYEQYFGPGTRLTVT | |
| | CDR1-b | DFQATT | 79 |
| | CDR2-b | SNEGSKA | 80 |
| | CDR3-b | CSARLWGLTYEQYF | 1313 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GPGTRLTVT | 85 |
| TCRA- | a-Variable | CAGAAGGAGGTGGAGCAGAATTCTGGACCCCTCAGTGTTCCAGA | 1314 |
| 221 | nucleotide | GGGAGCCATTGCCTCTCTCAACTGCACTTACAGTGACCGAGGTTC | |
| | | CCAGTCCTTCTTCTGGTACAGACAATATTCTGGGAAAAGCCCTGA | |
| | | GTTGATAATGTCCATATACTCCAATGGTGACAAAGAAGATGGAA | |
| | | GGTTTACAGCACAGCTCAATAAAGCCAGCCAGTATGTTTCTCTGC | |
| | | TCATCAGAGACTCCCAGCCCAGTGATTCAGCCACCTACCTCTGTG | |
| | | CCGTGAACACACCATATGGAAACAAACTGGTCTTTGGCGCAGGA | |
| | | ACCATTCTGAGAGTCAAGTCCT | |
| | Variable-a | QKEVEQNSGPLSVPEGAIASLNCTYSDRGSQSFFWYRQYSGKSPELI | 1315 |
| | | MSIYSNGDKEDGRFTAQLNKASQYVSLLIRDSQPSDSATYLCAVNTP | |
| | | YGNKLVFGAGTILRVKS | |
| | CDR1-a | DRGSQS | 12 |
| | CDR2-a | IYSNGD | 13 |
| | CDR3-a | CAVNTPYGNKLVF | 1316 |
| | FWR1-a | QKEVEQNSGPLSVPEGAIASLNCTYS | 15 |
| | FWR2-a | FFWYRQYSGKSPELIMS | 1304 |
| | FWR3-a | KEDGRFTAQLNKASQYVSLLIRDSQPSDSATYL | 17 |
| | FWR4-a | GAGTILRVKS | 18 |
| TCRB- | b-Variable | GGTGCTGTCGTCTCTCAACATCCGAGCTGGGTTATCTGTAAGAGT | 1317 |
| 222 | nucleotide | GGAACCTCTGTGAAGATCGAGTGCCGTTCCCTGGACTTTCAGGCC | |
| | | ACAACTATGTTTTGGTATCGTCAGTTCCCGAAACAGAGTCTCATG | |
| | | CTGATGGCAACTTCCAATGAGGGCTCCAAGGCCACATACGAGCA | |
| | | AGGCGTCGAGAAGGACAAGTTTCTCATCAACCATGCAAGCCTGA | |
| | | CCTTGTCCACTCTGACAGTGACCAGTGCCCATCCTGAAGACAGCA | |
| | | GCTTCTACATCTGCAGTGCTAGAGATCGGTGGGCAGGCTATGGCT | |
| | | ACACCTTCGGTTCGGGGACCAGGTTAACCGTTGTAG | |
| | Variable-b | GAVVSQHPSWVICKSGTSVKIECRSLDFQATTMFWYRQFPKQSLML | 1318 |
| | | MATSNEGSKATYEQGVEKDKFLINHASLTLSTLTVTSAHPEDSSFYIC | |
| | | SARDRWAGYGYTFGSGTRLTVV | |
| | CDR1-b | DFQATT | 79 |
| | CDR2-b | SNEGSKA | 80 |
| | CDR3-b | CSARDRWAGYGYTF | 1319 |
| | FWR1-b | GAVVSQHPSWVICKSGTSVKIECRSL | 82 |
| | FWR2-b | MFWYRQFPKQSLMLMAT | 83 |
| | FWR3-b | TYEQGVEKDKFLINHASLTLSTLTVTSAHPEDSSFYI | 84 |
| | FWR4-b | GSGTRLTVV | 103 |
| TCRA- | a-Variable | GCTCAGACAGTCACTCAGTCTCAACCAGAGATGTCTGTGCAGGAG | 1320 |
| 222 | nucleotide | GCAGAGACCGTGACCCTGAGCTGCACATATGACACCAGTGAGAG | |
| | | TGATTATTATTTATTCTGGTACAAGCAGCCTCCCAGCAGGCAGAT | |
| | | GATTCTCGTTATTCGCCAAGAAGCTTATAAGCAACAGAATGCAAC | |
| | | AGAGAATCGTTTCTCTGTGAACTTCCAGAAAGCAGCCAAATCCTT | |
| | | CAGTCTCAAGATCTCAGACTCACAGCTGGGGGATGCCGCGATGTA | |
| | | TTTCTGTGCCCAATACAGTAACAATAACAATGACATGCGCTTTGG | |
| | | AGCAGGGACCAGACTGACAGTAAAACCAA | |
| | Variable-a | AQTVTQSQPEMSVQEAETVTLSCTYDTSESDYYLFWYKQPPSRQMI | 1321 |
| | | LVIRQEAYKQQNATENRFSVNFQKAAKSFSLKISDSQLGDAAMYFC | |
| | | AQYSNNNNDMRFGAGTRLTVKP | |
| | CDR1-a | TSESDYY | 286 |
| | CDR2-a | QEAYKQQN | 287 |
| | CDR3-a | CAQYSNNNNDMRF | 1322 |
| | FWR1-a | AQTVTQSQPEMSVQEAETVTLSCTYD | 289 |
| | FWR2-a | LFWYKQPPSRQMIL VIR | 290 |
| | FWR3-a | ATENRFSVNFQKAAKSFSLKISDSQLGDAAMYF | 291 |
| | FWR4-a | GAGTRLTVKP | 685 |

TABLE 2
Summary of SEQ ID NOS:

| | Variable | Variable- | cdr1- | cdr2- | cdr3- | fwr1- | fwr2- | fwr3- | fwr4- | Variable | Variable- | cdr1- |
|---|---|---|---|---|---|---|---|---|---|---|---|---|
| Clone | nt-b | b | b | b | b | b | b | b | b | nt-a | a | a |
| TCR- | 1 | 2 | 3 | 4 | 5 | 6 | 7 | 8 | 9 | 10 | 11 | 12 |
| 1 | | | | | | | | | | | | |
| TCR- | 19 | 20 | 21 | 22 | 23 | 24 | 25 | 26 | 27 | 28 | 29 | 30 |
| 2 | | | | | | | | | | | | |
| TCR- | 37 | 38 | 39 | 40 | 41 | 42 | 43 | 44 | 45 | 46 | 47 | 48 |
| 3 | | | | | | | | | | | | |
| TCR- | 55 | 56 | 57 | 58 | 59 | 60 | 61 | 62 | 63 | 68 | 69 | 70 |
| 4.1 | | | | | | | | | | | | |
| TCR- | 64 | 65 | 21 | 22 | 66 | 24 | 25 | 26 | 67 | 68 | 69 | 70 |
| 4.2 | | | | | | | | | | | | |
| TCR- | 77 | 78 | 79 | 80 | 81 | 82 | 83 | 84 | 85 | 86 | 87 | 88 |
| 5 | | | | | | | | | | | | |
| TCR- | 95 | 96 | 97 | 98 | 99 | 100 | 101 | 102 | 103 | 104 | 105 | 106 |
| 6 | | | | | | | | | | | | |
| TCR- | 113 | 114 | 79 | 80 | 115 | 82 | 83 | 84 | 85 | 116 | 117 | 118 |
| 7 | | | | | | | | | | | | |
| TCR- | 125 | 126 | 127 | 128 | 129 | 130 | 131 | 132 | 67 | 133 | 134 | 135 |
| 8 | | | | | | | | | | | | |
| TCR- | 142 | 143 | 144 | 145 | 146 | 147 | 148 | 149 | 150 | 151 | 152 | 153 |
| 9 | | | | | | | | | | | | |
| TCR- | 159 | 160 | 161 | 162 | 163 | 164 | 165 | 166 | 103 | 175 | 176 | 12 |
| 10.1 | | | | | | | | | | | | |
| TCR- | 167 | 168 | 169 | 170 | 171 | 172 | 173 | 174 | 103 | 175 | 176 | 12 |
| 10.2 | | | | | | | | | | | | |
| TCR- | 179 | 180 | 181 | 182 | 183 | 184 | 185 | 186 | 27 | 187 | 188 | 118 |
| 11 | | | | | | | | | | | | |
| TCR- | 191 | 192 | 79 | 80 | 193 | 82 | 83 | 84 | 27 | 194 | 195 | 118 |
| 12 | | | | | | | | | | | | |
| TCR- | 197 | 198 | 199 | 200 | 201 | 202 | 203 | 204 | 150 | 205 | 206 | 207 |
| 13 | | | | | | | | | | | | |
| TCR- | 214 | 215 | 216 | 217 | 218 | 219 | 220 | 221 | 103 | 222 | 223 | 224 |
| 14 | | | | | | | | | | | | |
| TCR- | 231 | 232 | 21 | 22 | 233 | 24 | 25 | 26 | 67 | 234 | 235 | 236 |
| 15 | | | | | | | | | | | | |
| TCR- | 243 | 244 | 216 | 217 | 245 | 219 | 220 | 221 | 246 | 247 | 248 | 118 |
| 16 | | | | | | | | | | | | |
| TCR- | 250 | 251 | 252 | 253 | 254 | 42 | 255 | 256 | 85 | 257 | 258 | 118 |
| 17 | | | | | | | | | | | | |
| TCR- | 261 | 262 | 263 | 264 | 265 | 266 | 267 | 268 | 67 | 269 | 270 | 118 |
| 18 | | | | | | | | | | | | |
| TCR- | 273 | 274 | 275 | 276 | 277 | 278 | 279 | 280 | 85 | 284 | 285 | 286 |
| 19.1 | | | | | | | | | | | | |
| TCR- | 281 | 282 | 79 | 80 | 283 | 82 | 83 | 84 | 67 | 284 | 285 | 286 |
| 19.2 | | | | | | | | | | | | |
| TCR- | 293 | 294 | 295 | 296 | 297 | 298 | 299 | 300 | 103 | 301 | 302 | 303 |
| 20 | | | | | | | | | | | | |
| TCR- | 309 | 310 | 21 | 22 | 311 | 24 | 25 | 26 | 85 | 312 | 313 | 314 |
| 21 | | | | | | | | | | | | |
| TCR- | 321 | 322 | 216 | 217 | 323 | 219 | 220 | 221 | 9 | 324 | 325 | 12 |
| 22 | | | | | | | | | | | | |
| TCR- | 327 | 328 | 216 | 217 | 323 | 219 | 220 | 221 | 9 | 336 | 337 | 303 |
| 23.1 | | | | | | | | | | | | |
| TCR- | 329 | 330 | 331 | 332 | 333 | 334 | 131 | 335 | 103 | 336 | 337 | 303 |
| 23.2 | | | | | | | | | | | | |
| TCR- | 340 | 341 | 342 | 343 | 344 | 345 | 346 | 347 | 9 | 348 | 349 | 350 |
| 24 | | | | | | | | | | | | |
| TCR- | 357 | 358 | 144 | 145 | 359 | 147 | 148 | 149 | 63 | 360 | 361 | 303 |
| 25 | | | | | | | | | | | | |
| TCR- | 364 | 365 | 252 | 366 | 367 | 368 | 369 | 370 | 246 | 371 | 372 | 207 |
| 26 | | | | | | | | | | | | |
| TCR- | 375 | 376 | 216 | 217 | 377 | 219 | 220 | 221 | 9 | 378 | 379 | 224 |
| 27 | | | | | | | | | | | | |
| TCR- | 381 | 382 | 383 | 384 | 385 | 386 | 387 | 388 | 63 | 389 | 390 | 207 |
| 28 | | | | | | | | | | | | |
| TCR- | 393 | 394 | 395 | 396 | 397 | 398 | 399 | 400 | 103 | 401 | 402 | 403 |
| 29 | | | | | | | | | | | | |
| TCR- | 410 | 411 | 79 | 80 | 412 | 82 | 83 | 84 | 246 | 413 | 414 | 415 |
| 30 | | | | | | | | | | | | |
| TCR- | 421 | 422 | 383 | 384 | 423 | 386 | 424 | 388 | 9 | 425 | 426 | 427 |
| 31 | | | | | | | | | | | | |
| TCR- | 433 | 434 | 97 | 98 | 435 | 100 | 101 | 102 | 45 | 436 | 437 | 88 |
| 32 | | | | | | | | | | | | |
| TCR- | 440 | 441 | 442 | 443 | 444 | 445 | 446 | 447 | 246 | 448 | 449 | 450 |
| 33 | | | | | | | | | | | | |
| TCR- | 457 | 458 | 342 | 343 | 459 | 345 | 346 | 347 | 103 | 463 | 464 | 118 |
| 34.1 | | | | | | | | | | | | |
| TCR- | 460 | 461 | 216 | 217 | 462 | 219 | 220 | 221 | 85 | 463 | 464 | 118 |
| 34.2 | | | | | | | | | | | | |
| TCR- | 466 | 467 | 468 | 469 | 470 | 471 | 472 | 473 | 63 | 247 | 248 | 118 |
| 35 | | | | | | | | | | | | |
| TCR- | 474 | 475 | 331 | 332 | 476 | 334 | 131 | 335 | 103 | 477 | 478 | 427 |
| 36 | | | | | | | | | | | | |
| TCR- | 481 | 482 | 169 | 170 | 483 | 172 | 173 | 174 | 67 | 484 | 485 | 486 |
| 37 | | | | | | | | | | | | |
| TCR- | 490 | 491 | 252 | 366 | 492 | 368 | 369 | 370 | 85 | 493 | 494 | 495 |
| 38 | | | | | | | | | | | | |
| TCR- | 501 | 502 | 252 | 366 | 503 | 368 | 369 | 370 | 63 | 504 | 505 | 506 |
| 39 | | | | | | | | | | | | |
| TCR- | 512 | 513 | 514 | 515 | 516 | 517 | 518 | 519 | 85 | 520 | 521 | 153 |
| 40 | | | | | | | | | | | | |
| TCR- | 523 | 524 | 525 | 526 | 527 | 528 | 529 | 530 | 246 | 531 | 532 | 533 |
| 41 | | | | | | | | | | | | |
| TCR- | 540 | 541 | 252 | 253 | 542 | 42 | 255 | 256 | 27 | 543 | 544 | 286 |
| 42 | | | | | | | | | | | | |
| TCR- | 547 | 548 | 252 | 253 | 549 | 42 | 255 | 256 | 85 | 550 | 551 | 552 |
| 43 | | | | | | | | | | | | |
| TCR- | 558 | 559 | 127 | 128 | 560 | 130 | 131 | 132 | 103 | 561 | 562 | 135 |
| 44 | | | | | | | | | | | | |
| TCR- | 564 | 565 | 216 | 217 | 566 | 219 | 220 | 221 | 67 | 567 | 568 | 135 |
| 45 | | | | | | | | | | | | |
| TCR- | 570 | 571 | 252 | 366 | 572 | 368 | 369 | 370 | 246 | 573 | 574 | 575 |
| 46 | | | | | | | | | | | | |
| TCR- | 581 | 582 | 216 | 217 | 583 | 219 | 220 | 221 | 103 | 584 | 585 | 118 |
| 47 | | | | | | | | | | | | |
| TCR- | 588 | 589 | 442 | 443 | 590 | 591 | 446 | 447 | 67 | 592 | 593 | 594 |
| 48 | | | | | | | | | | | | |
| TCR- | 601 | 602 | 97 | 98 | 603 | 100 | 101 | 102 | 9 | 604 | 605 | 575 |
| 49 | | | | | | | | | | | | |
| TCR- | 608 | 609 | 252 | 366 | 610 | 368 | 369 | 370 | 85 | 611 | 612 | 303 |
| 50 | | | | | | | | | | | | |
| TCR- | 614 | 615 | 295 | 296 | 616 | 298 | 299 | 300 | 9 | 617 | 618 | 450 |
| 51 | | | | | | | | | | | | |
| TCR- | 620 | 621 | 442 | 443 | 622 | 591 | 446 | 447 | 85 | 623 | 624 | 303 |
| 52 | | | | | | | | | | | | |
| TCR- | 627 | 628 | 263 | 629 | 630 | 631 | 632 | 633 | 27 | 634 | 635 | 594 |
| 53 | | | | | | | | | | | | |
| TCR- | 637 | 638 | 639 | 640 | 641 | 642 | 643 | 644 | 246 | 645 | 646 | 450 |
| 54 | | | | | | | | | | | | |
| TCR- | 648 | 649 | 79 | 80 | 650 | 82 | 83 | 84 | 9 | 651 | 652 | 314 |
| 55 | | | | | | | | | | | | |
| TCR- | 654 | 655 | 383 | 384 | 656 | 386 | 387 | 388 | 246 | 657 | 658 | 659 |
| 56 | | | | | | | | | | | | |
| TCR- | 663 | 664 | 665 | 666 | 667 | 668 | 669 | 670 | 246 | 671 | 672 | 673 |
| 57 | | | | | | | | | | | | |
| TCR- | 679 | 680 | 199 | 200 | 681 | 202 | 203 | 204 | 85 | 682 | 683 | 427 |
| 58 | | | | | | | | | | | | |
| TCR- | 686 | 687 | 468 | 469 | 688 | 471 | 472 | 473 | 9 | 689 | 690 | 118 |
| 59 | | | | | | | | | | | | |
| TCR- | 692 | 693 | 665 | 666 | 694 | 668 | 669 | 670 | 246 | 695 | 696 | 427 |
| 60 | | | | | | | | | | | | |
| TCR- | 698 | 699 | 79 | 80 | 700 | 82 | 83 | 84 | 85 | 701 | 702 | 594 |
| 61 | | | | | | | | | | | | |
| TCR- | 704 | 705 | 169 | 706 | 707 | 172 | 173 | 708 | 9 | 709 | 710 | 506 |
| 62 | | | | | | | | | | | | |
| TCR- | 712 | 713 | 252 | 253 | 714 | 42 | 255 | 256 | 85 | 715 | 716 | 207 |
| 63 | | | | | | | | | | | | |
| TCR- | 718 | 719 | 331 | 332 | 720 | 334 | 131 | 335 | 9 | 721 | 722 | 106 |
| 64 | | | | | | | | | | | | |
| TCR- | 725 | 726 | 127 | 128 | 727 | 130 | 131 | 132 | 9 | 728 | 729 | 118 |
| 65 | | | | | | | | | | | | |
| TCR- | 731 | 732 | 442 | 443 | 733 | 591 | 446 | 447 | 246 | 734 | 735 | 552 |
| 66 | | | | | | | | | | | | |
| TCR- | 737 | 738 | 442 | 443 | 739 | 591 | 446 | 447 | 85 | 740 | 741 | 207 |
| 67 | | | | | | | | | | | | |
| TCR- | 743 | 744 | 263 | 629 | 745 | 631 | 746 | 747 | 85 | 748 | 749 | 207 |
| 68 | | | | | | | | | | | | |
| TCR- | 751 | 752 | 383 | 384 | 753 | 386 | 387 | 388 | 27 | 754 | 755 | 450 |
| 69 | | | | | | | | | | | | |
| TCR- | 757 | 758 | 331 | 332 | 759 | 334 | 131 | 335 | 63 | 760 | 761 | 762 |
| 70 | | | | | | | | | | | | |
| TCR- | 768 | 769 | 169 | 170 | 770 | 172 | 173 | 174 | 63 | 771 | 772 | 314 |
| 71 | | | | | | | | | | | | |
| TCR- | 775 | 776 | 216 | 217 | 777 | 219 | 220 | 221 | 85 | 778 | 779 | 207 |
| 72 | | | | | | | | | | | | |
| TCR- | 781 | 782 | 665 | 666 | 783 | 668 | 669 | 670 | 45 | 784 | 785 | 118 |
| 73 | | | | | | | | | | | | |
| TCR- | 788 | 789 | 275 | 443 | 790 | 791 | 792 | 793 | 63 | 794 | 795 | 314 |
| 74 | | | | | | | | | | | | |
| TCR- | 797 | 798 | 199 | 200 | 799 | 202 | 203 | 204 | 27 | 800 | 801 | 450 |
| 75 | | | | | | | | | | | | |
| TCR- | 803 | 804 | 97 | 98 | 805 | 100 | 101 | 102 | 9 | 806 | 807 | 12 |
| 76.1 | | | | | | | | | | | | |
| TCR- | 803 | 804 | 97 | 98 | 805 | 100 | 101 | 102 | 9 | 809 | 810 | 286 |
| 76.2 | | | | | | | | | | | | |
| TCR- | 813 | 814 | 144 | 145 | 815 | 147 | 148 | 149 | 816 | 817 | 818 | 88 |
| 77 | | | | | | | | | | | | |
| TCR- | 820 | 821 | 442 | 443 | 822 | 591 | 446 | 447 | 150 | 823 | 824 | 403 |
| 78 | | | | | | | | | | | | |
| TCR- | 826 | 827 | 21 | 22 | 828 | 24 | 25 | 26 | 27 | 829 | 830 | 831 |
| 79 | | | | | | | | | | | | |
| TCR- | 837 | 838 | 383 | 384 | 839 | 386 | 387 | 388 | 103 | 840 | 841 | 762 |
| 80 | | | | | | | | | | | | |
| TCR- | 843 | 844 | 845 | 846 | 847 | 848 | 849 | 850 | 63 | 851 | 852 | 30 |
| 81 | | | | | | | | | | | | |
| TCR- | 856 | 857 | 858 | 859 | 860 | 861 | 862 | 863 | 27 | 864 | 865 | 673 |
| 82 | | | | | | | | | | | | |
| TCR- | 868 | 869 | 342 | 343 | 870 | 345 | 346 | 347 | 871 | 872 | 873 | 314 |
| 83 | | | | | | | | | | | | |
| TCR- | 875 | 876 | 216 | 217 | 877 | 219 | 220 | 221 | 85 | 878 | 879 | 303 |
| 84 | | | | | | | | | | | | |
| TCR- | 881 | 882 | 216 | 217 | 883 | 219 | 220 | 221 | 9 | 884 | 885 | 886 |
| 85 | | | | | | | | | | | | |
| TCR- | 892 | 893 | 894 | 895 | 896 | 897 | 898 | 899 | 85 | 900 | 901 | 207 |
| 86 | | | | | | | | | | | | |
| TCR- | 904 | 905 | 216 | 217 | 906 | 219 | 220 | 221 | 9 | 907 | 908 | 48 |
| 87 | | | | | | | | | | | | |
| TCR- | 911 | 912 | 57 | 58 | 913 | 60 | 61 | 62 | 9 | 914 | 915 | 506 |
| 88 | | | | | | | | | | | | |
| TCR- | 917 | 918 | 199 | 200 | 919 | 202 | 203 | 204 | 103 | 920 | 921 | 236 |
| 89 | | | | | | | | | | | | |
| TCR- | 923 | 924 | 252 | 253 | 925 | 42 | 255 | 256 | 9 | 926 | 927 | 533 |
| 90 | | | | | | | | | | | | |
| TCR- | 929 | 930 | 442 | 443 | 931 | 591 | 446 | 447 | 27 | 935 | 936 | 207 |
| 91.1 | | | | | | | | | | | | |
| TCR- | 932 | 933 | 639 | 640 | 934 | 642 | 643 | 644 | 45 | 935 | 936 | 207 |
| 91.2 | | | | | | | | | | | | |
| TCR- | 938 | 939 | 79 | 80 | 940 | 82 | 83 | 84 | 246 | 941 | 942 | 286 |
| 92 | | | | | | | | | | | | |
| TCR- | 944 | 945 | 395 | 396 | 946 | 398 | 399 | 400 | 9 | 947 | 948 | 207 |
| 93 | | | | | | | | | | | | |
| TCR- | 950 | 951 | 275 | 276 | 952 | 278 | 279 | 280 | 9 | 953 | 954 | 118 |
| 94 | | | | | | | | | | | | |
| TCR- | 956 | 957 | 639 | 640 | 958 | 642 | 643 | 644 | 9 | 959 | 960 | 207 |
| 95 | | | | | | | | | | | | |
| TCR- | 962 | 963 | 383 | 384 | 964 | 386 | 424 | 388 | 67 | 965 | 966 | 415 |
| 96 | | | | | | | | | | | | |
| TCR- | 968 | 969 | 57 | 58 | 970 | 60 | 61 | 62 | 9 | 971 | 972 | 973 |
| 97 | | | | | | | | | | | | |
| TCR- | 979 | 980 | 514 | 515 | 981 | 517 | 518 | 519 | 85 | 982 | 983 | 118 |
| 98 | | | | | | | | | | | | |
| TCR- | 985 | 986 | 514 | 515 | 987 | 517 | 518 | 519 | 85 | 988 | 989 | 70 |
| 99 | | | | | | | | | | | | |
| TCR- | 991 | 992 | 383 | 384 | 993 | 386 | 424 | 388 | 103 | 994 | 995 | 506 |
| 100 | | | | | | | | | | | | |
| TCR- | 997 | 998 | 252 | 366 | 999 | 368 | 369 | 370 | 85 | 1000 | 1001 | 118 |
| 101 | | | | | | | | | | | | |
| TCR- | 1003 | 1004 | 1005 | 276 | 1006 | 1007 | 1008 | 1009 | 9 | 1010 | 1011 | 506 |
| 102 | | | | | | | | | | | | |
| TCR- | 1013 | 1014 | 97 | 98 | 1015 | 100 | 101 | 102 | 85 | 1016 | 1017 | 12 |
| 103 | | | | | | | | | | | | |
| TCR- | 679 | 680 | 199 | 200 | 681 | 202 | 203 | 204 | 85 | 682 | 683 | 427 |
| 104 | | | | | | | | | | | | |
| TCR- | 1019 | 1020 | 383 | 384 | 1021 | 386 | 387 | 388 | 816 | 1022 | 1023 | 153 |
| 105 | | | | | | | | | | | | |
| TCR- | 1026 | 1027 | 252 | 366 | 1028 | 368 | 369 | 370 | 63 | 1029 | 1030 | 831 |
| 106 | | | | | | | | | | | | |
| TCR- | 1032 | 1033 | 383 | 384 | 1034 | 386 | 387 | 388 | 9 | 1035 | 1036 | 303 |
| 107 | | | | | | | | | | | | |
| TCR- | 1038 | 1039 | 383 | 384 | 1040 | 386 | 387 | 388 | 9 | 1041 | 1042 | 403 |
| 108 | | | | | | | | | | | | |
| TCR- | 1044 | 1045 | 252 | 1046 | 1047 | 1048 | 1049 | 1050 | 85 | 1051 | 1052 | 506 |
| 109 | | | | | | | | | | | | |
| TCR- | 1054 | 1055 | 169 | 706 | 1056 | 172 | 173 | 708 | 85 | 1057 | 1058 | 533 |
| 110 | | | | | | | | | | | | |
| TCR- | 1060 | 1061 | 79 | 80 | 1062 | 1063 | 83 | 84 | 85 | 1064 | 1065 | 106 |
| 111 | | | | | | | | | | | | |
| TCR- | 1067 | 1068 | 525 | 526 | 1069 | 528 | 529 | 530 | 9 | 1070 | 1071 | 1072 |
| 112 | | | | | | | | | | | | |
| TCR- | 1078 | 1079 | 442 | 443 | 1080 | 591 | 446 | 447 | 1081 | 1082 | 1083 | 673 |
| 113 | | | | | | | | | | | | |
| TCR- | 1085 | 1086 | 216 | 217 | 1087 | 219 | 220 | 221 | 27 | 1088 | 1089 | 12 |
| 114 | | | | | | | | | | | | |
| TCR- | 1091 | 1092 | 252 | 253 | 1093 | 42 | 255 | 256 | 85 | 1094 | 1095 | 552 |
| 115 | | | | | | | | | | | | |
| TCR- | 1097 | 1098 | 216 | 217 | 1099 | 219 | 220 | 221 | 9 | 1100 | 1101 | 70 |
| 116 | | | | | | | | | | | | |
| TCR- | 1104 | 1105 | 442 | 443 | 1106 | 445 | 446 | 447 | 871 | 1107 | 1108 | 48 |
| 117 | | | | | | | | | | | | |
| TCR- | 1110 | 1111 | 442 | 443 | 1112 | 445 | 446 | 447 | 246 | 1113 | 1114 | 1115 |
| 118 | | | | | | | | | | | | |
| TCR- | 1117 | 1118 | 468 | 469 | 1119 | 471 | 472 | 473 | 27 | 1120 | 1121 | 552 |
| 119 | | | | | | | | | | | | |
| TCR- | 1123 | 1124 | 1125 | 1126 | 1127 | 1128 | 1129 | 1130 | 9 | 1131 | 1132 | 886 |
| 120 | | | | | | | | | | | | |
| TCR- | 1135 | 1136 | 383 | 384 | 1137 | 386 | 387 | 388 | 85 | 1138 | 1139 | 48 |
| 121 | | | | | | | | | | | | |
| TCR- | 1141 | 1142 | 252 | 366 | 1143 | 368 | 369 | 370 | 9 | 1144 | 1145 | 118 |
| 122 | | | | | | | | | | | | |
| TCR- | 1147 | 1148 | 514 | 515 | 1149 | 517 | 518 | 519 | 9 | 1150 | 1151 | 106 |
| 123 | | | | | | | | | | | | |
| TCR- | 1153 | 1154 | 514 | 515 | 1155 | 517 | 518 | 519 | 103 | 1156 | 1157 | 118 |
| 124 | | | | | | | | | | | | |
| TCR- | 1159 | 1160 | 331 | 332 | 1161 | 334 | 131 | 335 | 9 | 1162 | 1163 | 552 |
| 125 | | | | | | | | | | | | |
| TCR- | 1165 | 1166 | 894 | 1167 | 1168 | 1169 | 1170 | 1171 | 9 | 1172 | 1173 | 106 |
| 126 | | | | | | | | | | | | |
| TCR- | 1175 | 1176 | 383 | 384 | 1177 | 386 | 387 | 388 | 103 | 1178 | 1179 | 450 |
| 127 | | | | | | | | | | | | |
| TCR- | 1182 | 1183 | 252 | 1046 | 1184 | 1048 | 1049 | 1050 | 9 | 1185 | 1186 | 314 |
| 128 | | | | | | | | | | | | |
| TCR- | 1188 | 1189 | 79 | 80 | 1190 | 1063 | 83 | 84 | 103 | 1191 | 1192 | 762 |
| 129 | | | | | | | | | | | | |
| TCR- | 1194 | 1195 | 468 | 469 | 1196 | 471 | 472 | 473 | 85 | 1197 | 1198 | 48 |
| 130 | | | | | | | | | | | | |
| TCR- | 1200 | 1201 | 252 | 253 | 1202 | 42 | 255 | 256 | 246 | 1203 | 1204 | 207 |
| 131 | | | | | | | | | | | | |
| TCR- | 1206 | 1207 | 252 | 253 | 1208 | 42 | 255 | 256 | 9 | 1209 | 1210 | 118 |
| 132 | | | | | | | | | | | | |
| TCR- | 1212 | 1213 | 97 | 98 | 1214 | 100 | 101 | 102 | 67 | 1215 | 1216 | 973 |
| 133 | | | | | | | | | | | | |
| TCR- | 1218 | 1219 | 39 | 40 | 1220 | 42 | 43 | 44 | 9 | 1221 | 1222 | 450 |
| 134 | | | | | | | | | | | | |
| TCR- | 1224 | 1225 | 3 | 4 | 1226 | 6 | 7 | 8 | 9 | 1227 | 1228 | 533 |
| 135 | | | | | | | | | | | | |
| TCR- | 1230 | 1231 | 514 | 515 | 1232 | 517 | 518 | 519 | 27 | 1233 | 1234 | 506 |
| 136 | | | | | | | | | | | | |
| TCR- | 1236 | 1237 | 97 | 98 | 1238 | 100 | 101 | 102 | 871 | 1239 | 1240 | 1072 |
| 137 | | | | | | | | | | | | |
| TCR- | 1242 | 1243 | 252 | 253 | 1244 | 42 | 255 | 256 | 103 | 1245 | 1246 | 495 |
| 138 | | | | | | | | | | | | |
| TCR- | 1249 | 1250 | 216 | 217 | 1251 | 219 | 220 | 221 | 27 | 1252 | 1253 | 207 |
| 139 | | | | | | | | | | | | |
| TCR- | 1255 | 1256 | 894 | 895 | 1257 | 897 | 898 | 899 | 9 | 1258 | 1259 | 224 |
| 140 | | | | | | | | | | | | |
| TCR- | 1262 | 1263 | 252 | 1046 | 1264 | 1048 | 1049 | 1050 | 67 | 1265 | 1266 | 427 |
| 141 | | | | | | | | | | | | |
| TCR- | 1268 | 1269 | 639 | 640 | 1270 | 642 | 643 | 644 | 45 | 1271 | 1272 | 314 |
| 142 | | | | | | | | | | | | |
| TCR- | 1274 | 1275 | 468 | 469 | 1276 | 471 | 472 | 473 | 85 | 1277 | 1278 | 224 |
| 143 | | | | | | | | | | | | |
| TCR- | 1280 | 1281 | 79 | 80 | 1282 | 82 | 83 | 84 | 9 | 1283 | 1284 | 673 |
| 144 | | | | | | | | | | | | |
| TCR- | 1286 | 1287 | 79 | 80 | 1288 | 82 | 83 | 84 | 246 | 1289 | 1290 | 506 |
| 145 | | | | | | | | | | | | |
| TCR- | 1292 | 1293 | 79 | 80 | 1294 | 82 | 83 | 84 | 246 | 1295 | 1296 | 450 |
| 146 | | | | | | | | | | | | |
| TCR- | 1298 | 1299 | 21 | 22 | 1300 | 24 | 25 | 26 | 27 | 1301 | 1302 | 12 |
| 147 | | | | | | | | | | | | |
| TCR- | 1305 | 1306 | 216 | 217 | 1307 | 219 | 220 | 221 | 9 | 1308 | 1309 | 673 |
| 148 | | | | | | | | | | | | |
| TCR- | 1311 | 1312 | 79 | 80 | 1313 | 82 | 83 | 84 | 85 | 1314 | 1315 | 12 |
| 149 | | | | | | | | | | | | |
| TCR- | 1317 | 1318 | 79 | 80 | 1319 | 82 | 83 | 84 | 103 | 1320 | 1321 | 286 |
| 150 | | | | | | | | | | | | |
| TCR- | 1323 | 1324 | 216 | 217 | 1325 | 219 | 220 | 221 | 103 | 1326 | 1327 | 70 |
| 151 | | | | | | | | | | | | |
| TCR- | 1329 | 1330 | 169 | 170 | 1331 | 172 | 173 | 174 | 63 | 1332 | 1333 | 236 |
| 152 | | | | | | | | | | | | |
| TCR- | 1335 | 1336 | 894 | 895 | 1337 | 897 | 898 | 899 | 9 | 1338 | 1339 | 575 |
| 153 | | | | | | | | | | | | |
| TCR- | 1341 | 1342 | 514 | 515 | 1343 | 517 | 518 | 519 | 9 | 1344 | 1345 | 118 |
| 154.1 | | | | | | | | | | | | |
| TCR- | 1341 | 1342 | 514 | 515 | 1343 | 517 | 518 | 519 | 9 | 1347 | 1348 | 552 |
| 154.2 | | | | | | | | | | | | |
| TCR- | 501 | 502 | 252 | 366 | 503 | 368 | 369 | 370 | 63 | 504 | 505 | 506 |
| 155 | | | | | | | | | | | | |
| TCR- | 512 | 513 | 514 | 515 | 516 | 517 | 518 | 519 | 85 | 520 | 521 | 153 |
| 156 | | | | | | | | | | | | |
| TCR- | 523 | 524 | 525 | 526 | 527 | 528 | 529 | 530 | 246 | 531 | 532 | 533 |
| 157 | | | | | | | | | | | | |
| TCR- | 540 | 541 | 252 | 253 | 542 | 42 | 255 | 256 | 27 | 543 | 544 | 286 |
| 158 | | | | | | | | | | | | |
| TCR- | 547 | 548 | 252 | 253 | 549 | 42 | 255 | 256 | 85 | 550 | 551 | 552 |
| 159 | | | | | | | | | | | | |
| TCR- | 558 | 559 | 127 | 128 | 560 | 130 | 131 | 132 | 103 | 561 | 562 | 135 |
| 160 | | | | | | | | | | | | |
| TCR- | 564 | 565 | 216 | 217 | 566 | 219 | 220 | 221 | 67 | 567 | 568 | 135 |
| 161 | | | | | | | | | | | | |
| TCR- | 570 | 571 | 252 | 366 | 572 | 368 | 369 | 370 | 246 | 573 | 574 | 575 |
| 162 | | | | | | | | | | | | |
| TCR- | 581 | 582 | 216 | 217 | 583 | 219 | 220 | 221 | 103 | 584 | 585 | 118 |
| 163 | | | | | | | | | | | | |
| TCR- | 588 | 589 | 442 | 443 | 590 | 591 | 446 | 447 | 67 | 592 | 593 | 594 |
| 164 | | | | | | | | | | | | |
| TCR- | 601 | 602 | 97 | 98 | 603 | 100 | 101 | 102 | 9 | 604 | 605 | 575 |
| 165 | | | | | | | | | | | | |
| TCR- | 608 | 609 | 252 | 366 | 610 | 368 | 369 | 370 | 85 | 611 | 612 | 303 |
| 166 | | | | | | | | | | | | |
| TCR- | 614 | 615 | 295 | 296 | 616 | 298 | 299 | 300 | 9 | 617 | 618 | 450 |
| 167 | | | | | | | | | | | | |
| TCR- | 620 | 621 | 442 | 443 | 622 | 591 | 446 | 447 | 85 | 623 | 624 | 303 |
| 168 | | | | | | | | | | | | |
| TCR- | 627 | 628 | 263 | 629 | 630 | 631 | 632 | 633 | 27 | 634 | 635 | 594 |
| 169 | | | | | | | | | | | | |
| TCR- | 637 | 638 | 639 | 640 | 641 | 642 | 643 | 644 | 246 | 645 | 646 | 450 |
| 170 | | | | | | | | | | | | |
| TCR- | 648 | 649 | 79 | 80 | 650 | 82 | 83 | 84 | 9 | 651 | 652 | 314 |
| 171 | | | | | | | | | | | | |
| TCR- | 654 | 655 | 383 | 384 | 656 | 386 | 387 | 388 | 246 | 657 | 658 | 659 |
| 172 | | | | | | | | | | | | |
| TCR- | 663 | 664 | 665 | 666 | 667 | 668 | 669 | 670 | 246 | 671 | 672 | 673 |
| 173 | | | | | | | | | | | | |
| TCR- | 679 | 680 | 199 | 200 | 681 | 202 | 203 | 204 | 85 | 682 | 683 | 427 |
| 174 | | | | | | | | | | | | |
| TCR- | 686 | 687 | 468 | 469 | 688 | 471 | 472 | 473 | 9 | 689 | 690 | 118 |
| 175 | | | | | | | | | | | | |
| TCR- | 692 | 693 | 665 | 666 | 694 | 668 | 669 | 670 | 246 | 695 | 696 | 427 |
| 176 | | | | | | | | | | | | |
| TCR- | 698 | 699 | 79 | 80 | 700 | 82 | 83 | 84 | 85 | 701 | 702 | 594 |
| 177 | | | | | | | | | | | | |
| TCR- | 704 | 705 | 169 | 706 | 707 | 172 | 173 | 708 | 9 | 709 | 710 | 506 |
| 178 | | | | | | | | | | | | |
| TCR- | 712 | 713 | 252 | 253 | 714 | 42 | 255 | 256 | 85 | 715 | 716 | 207 |
| 179 | | | | | | | | | | | | |
| TCR- | 775 | 776 | 216 | 217 | 777 | 219 | 220 | 221 | 85 | 779 | 779 | 207 |
| 180 | | | | | | | | | | | | |
| TCR- | 781 | 782 | 665 | 666 | 783 | 668 | 669 | 670 | 45 | 784 | 785 | 118 |
| 181 | | | | | | | | | | | | |
| TCR- | 788 | 789 | 275 | 443 | 790 | 791 | 792 | 793 | 63 | 794 | 795 | 314 |
| 182 | | | | | | | | | | | | |
| TCR- | 797 | 798 | 199 | 200 | 799 | 202 | 203 | 204 | 27 | 800 | 801 | 450 |
| 183 | | | | | | | | | | | | |
| TCR- | 803 | 804 | 97 | 98 | 805 | 100 | 101 | 102 | 9 | 806 | 807 | 12 |
| 184.1 | | | | | | | | | | | | |
| TCR- | 803 | 804 | 97 | 98 | 805 | 100 | 101 | 102 | 9 | 809 | 810 | 286 |
| 184.2 | | | | | | | | | | | | |
| TCR- | 813 | 814 | 144 | 145 | 815 | 147 | 148 | 149 | 816 | 817 | 818 | 88 |
| 185 | | | | | | | | | | | | |
| TCR- | 820 | 821 | 442 | 443 | 822 | 591 | 446 | 447 | 150 | 823 | 824 | 403 |
| 186 | | | | | | | | | | | | |
| TCR- | 826 | 827 | 21 | 22 | 828 | 24 | 25 | 26 | 27 | 829 | 830 | 831 |
| 187 | | | | | | | | | | | | |
| TCR- | 904 | 905 | 216 | 217 | 906 | 219 | 220 | 221 | 9 | 907 | 908 | 48 |
| 188 | | | | | | | | | | | | |
| TCR- | 911 | 912 | 57 | 58 | 913 | 60 | 61 | 62 | 9 | 914 | 915 | 506 |
| 189 | | | | | | | | | | | | |
| TCR- | 917 | 918 | 199 | 200 | 919 | 202 | 203 | 204 | 103 | 920 | 921 | 236 |
| 190 | | | | | | | | | | | | |
| TCR- | 923 | 924 | 252 | 253 | 925 | 42 | 255 | 256 | 9 | 926 | 927 | 533 |
| 191 | | | | | | | | | | | | |
| TCR- | 929 | 930 | 442 | 443 | 931 | 591 | 446 | 447 | 27 | 935 | 936 | 207 |
| 192.1 | | | | | | | | | | | | |
| TCR- | 932 | 933 | 639 | 640 | 934 | 642 | 643 | 644 | 45 | 935 | 936 | 207 |
| 192.2 | | | | | | | | | | | | |
| TCR- | 938 | 939 | 79 | 80 | 940 | 82 | 83 | 84 | 246 | 941 | 942 | 286 |
| 193 | | | | | | | | | | | | |
| TCR- | 679 | 680 | 199 | 200 | 681 | 202 | 203 | 204 | 85 | 682 | 683 | 427 |
| 194 | | | | | | | | | | | | |
| TCR- | 1019 | 1020 | 383 | 384 | 1021 | 386 | 387 | 388 | 816 | 1022 | 1023 | 153 |
| 195 | | | | | | | | | | | | |
| TCR- | 1026 | 1027 | 252 | 366 | 1028 | 368 | 369 | 370 | 63 | 1029 | 1030 | 831 |
| 196 | | | | | | | | | | | | |
| TCR- | 1032 | 1033 | 383 | 384 | 1034 | 386 | 387 | 388 | 9 | 1035 | 1036 | 303 |
| 197 | | | | | | | | | | | | |
| TCR- | 1038 | 1039 | 383 | 384 | 1040 | 386 | 387 | 388 | 9 | 1041 | 1042 | 403 |
| 198 | | | | | | | | | | | | |
| TCR- | 1044 | 1045 | 252 | 1046 | 1047 | 1048 | 1049 | 1050 | 85 | 1051 | 1052 | 506 |
| 199 | | | | | | | | | | | | |
| TCR- | 1054 | 1055 | 169 | 706 | 1056 | 172 | 173 | 708 | 85 | 1057 | 1058 | 533 |
| 200 | | | | | | | | | | | | |
| TCR- | 1060 | 1061 | 79 | 80 | 1062 | 1063 | 83 | 84 | 85 | 1064 | 1065 | 106 |
| 201 | | | | | | | | | | | | |
| TCR- | 1067 | 1068 | 525 | 526 | 1069 | 528 | 529 | 530 | 9 | 1070 | 1071 | 1072 |
| 202 | | | | | | | | | | | | |
| TCR- | 1078 | 1079 | 442 | 443 | 1080 | 591 | 446 | 447 | 1081 | 1082 | 1083 | 673 |
| 203 | | | | | | | | | | | | |
| TCR- | 1085 | 1086 | 216 | 217 | 1087 | 219 | 220 | 221 | 27 | 1088 | 1089 | 12 |
| 204 | | | | | | | | | | | | |
| TCR- | 1091 | 1092 | 252 | 253 | 1093 | 42 | 255 | 256 | 85 | 1094 | 1095 | 552 |
| 205 | | | | | | | | | | | | |
| TCR- | 1097 | 1098 | 216 | 217 | 1099 | 219 | 220 | 221 | 9 | 1100 | 1101 | 70 |
| 206 | | | | | | | | | | | | |
| TCR- | 1104 | 1105 | 442 | 443 | 1106 | 445 | 446 | 447 | 871 | 1107 | 1108 | 48 |
| 207 | | | | | | | | | | | | |
| TCR- | 1110 | 1111 | 442 | 443 | 1112 | 445 | 446 | 447 | 246 | 1113 | 1114 | 1115 |
| 208 | | | | | | | | | | | | |
| TCR- | 1117 | 1118 | 468 | 469 | 1119 | 471 | 472 | 473 | 27 | 1120 | 1121 | 552 |
| 209 | | | | | | | | | | | | |
| TCR- | 1123 | 1124 | 1125 | 1126 | 1127 | 1128 | 1129 | 1130 | 9 | 1131 | 1132 | 886 |
| 210 | | | | | | | | | | | | |
| TCR- | 1135 | 1136 | 383 | 384 | 1137 | 386 | 387 | 388 | 85 | 1138 | 1139 | 48 |
| 211 | | | | | | | | | | | | |
| TCR- | 1141 | 1142 | 252 | 366 | 1143 | 368 | 369 | 370 | 9 | 1144 | 1145 | 118 |
| 212 | | | | | | | | | | | | |
| TCR- | 1262 | 1263 | 252 | 1046 | 1264 | 1048 | 1049 | 1050 | 67 | 1265 | 1266 | 427 |
| 213 | | | | | | | | | | | | |
| TCR- | 1268 | 1269 | 639 | 640 | 1270 | 642 | 643 | 644 | 45 | 1271 | 1272 | 314 |
| 214 | | | | | | | | | | | | |
| TCR- | 1274 | 1275 | 468 | 469 | 1276 | 471 | 472 | 473 | 85 | 1277 | 1278 | 224 |
| 215 | | | | | | | | | | | | |
| TCR- | 1280 | 1281 | 79 | 80 | 1282 | 82 | 83 | 84 | 9 | 1283 | 1284 | 673 |
| 216 | | | | | | | | | | | | |
| TCR- | 1286 | 1287 | 79 | 80 | 1288 | 82 | 83 | 84 | 246 | 1289 | 1290 | 506 |
| 217 | | | | | | | | | | | | |
| TCR- | 1292 | 1293 | 79 | 80 | 1294 | 82 | 83 | 84 | 246 | 1295 | 1296 | 450 |
| 218 | | | | | | | | | | | | |
| TCR- | 1298 | 1299 | 21 | 22 | 1300 | 24 | 25 | 26 | 27 | 1301 | 1302 | 12 |
| 219 | | | | | | | | | | | | |
| TCR- | 1305 | 1306 | 216 | 217 | 1307 | 219 | 220 | 221 | 9 | 1308 | 1309 | 673 |
| 220 | | | | | | | | | | | | |
| TCR- | 1311 | 1312 | 79 | 80 | 1313 | 82 | 83 | 84 | 85 | 1314 | 1315 | 12 |
| 221 | | | | | | | | | | | | |
| TCR- | 1317 | 1318 | 79 | 80 | 1319 | 82 | 83 | 84 | 103 | 1320 | 1321 | 286 |
| 222 | | | | | | | | | | | | |

| | cdr2- | cdr3- | fwr1- | fwr2- | fwr3- | fwr4- |
|---|---|---|---|---|---|---|
| Clone | a | a | a | a | a | a |
| TCR- | 13 | 14 | 15 | 16 | 17 | 18 |
| 1 | | | | | | |
| TCR- | 31 | 32 | 33 | 34 | 35 | 36 |
| 2 | | | | | | |
| TCR- | 49 | 50 | 51 | 52 | 53 | 54 |
| 3 | | | | | | |
| TCR- | 71 | 72 | 73 | 74 | 75 | 76 |
| 4.1 | | | | | | |
| TCR- | 71 | 72 | 73 | 74 | 75 | 76 |
| 4.2 | | | | | | |
| TCR- | 89 | 90 | 91 | 92 | 93 | 94 |
| 5 | | | | | | |
| TCR- | 107 | 108 | 109 | 110 | 111 | 112 |
| 6 | | | | | | |
| TCR- | 119 | 120 | 121 | 122 | 123 | 124 |
| 7 | | | | | | |
| TCR- | 136 | 137 | 138 | 139 | 140 | 141 |
| 8 | | | | | | |
| TCR- | 154 | 155 | 156 | 157 | 158 | 141 |
| 9 | | | | | | |
| TCR- | 13 | 177 | 15 | 16 | 17 | 178 |
| 10.1 | | | | | | |
| TCR- | 13 | 177 | 15 | 16 | 17 | 178 |
| 10.2 | | | | | | |
| TCR- | 119 | 189 | 121 | 122 | 123 | 190 |
| 11 | | | | | | |
| TCR- | 119 | 196 | 121 | 122 | 123 | 141 |
| 12 | | | | | | |
| TCR- | 208 | 209 | 210 | 211 | 212 | 213 |
| 13 | | | | | | |
| TCR- | 225 | 226 | 227 | 228 | 229 | 230 |
| 14 | | | | | | |
| TCR- | 237 | 238 | 239 | 240 | 241 | 242 |
| 15 | | | | | | |
| TCR- | 119 | 249 | 121 | 122 | 123 | 18 |
| 16 | | | | | | |
| TCR- | 119 | 259 | 121 | 122 | 123 | 260 |
| 17 | | | | | | |
| TCR- | 119 | 271 | 121 | 122 | 123 | 272 |
| 18 | | | | | | |
| TCR- | 287 | 288 | 289 | 290 | 291 | 292 |
| 19.1 | | | | | | |
| TCR- | 287 | 288 | 289 | 290 | 291 | 292 |
| 19.2 | | | | | | |
| TCR- | 304 | 305 | 306 | 307 | 308 | 213 |
| 20 | | | | | | |
| TCR- | 315 | 316 | 317 | 318 | 319 | 320 |
| 21 | | | | | | |
| TCR- | 13 | 326 | 15 | 16 | 17 | 94 |
| 22 | | | | | | |
| TCR- | 304 | 338 | 306 | 307 | 308 | 339 |
| 23.1 | | | | | | |
| TCR- | 304 | 338 | 306 | 307 | 308 | 339 |
| 23.2 | | | | | | |
| TCR- | 351 | 352 | 353 | 354 | 355 | 356 |
| 24 | | | | | | |
| TCR- | 304 | 362 | 306 | 307 | 308 | 363 |
| 25 | | | | | | |
| TCR- | 208 | 373 | 210 | 374 | 212 | 213 |
| 26 | | | | | | |
| TCR- | 225 | 380 | 227 | 228 | 229 | 36 |
| 27 | | | | | | |
| TCR- | 208 | 391 | 210 | 211 | 212 | 392 |
| 28 | | | | | | |
| TCR- | 404 | 405 | 406 | 407 | 408 | 409 |
| 29 | | | | | | |
| TCR- | 416 | 417 | 418 | 419 | 420 | 213 |
| 30 | | | | | | |
| TCR- | 428 | 429 | 430 | 431 | 432 | 363 |
| 31 | | | | | | |
| TCR- | 89 | 438 | 439 | 92 | 93 | 213 |
| 32 | | | | | | |
| TCR- | 451 | 452 | 453 | 454 | 455 | 456 |
| 33 | | | | | | |
| TCR- | 119 | 465 | 121 | 122 | 123 | 272 |
| 34.1 | | | | | | |
| TCR- | 119 | 465 | 121 | 122 | 123 | 272 |
| 34.2 | | | | | | |
| TCR- | 119 | 249 | 121 | 122 | 123 | 18 |
| 35 | | | | | | |
| TCR- | 428 | 479 | 430 | 431 | 432 | 480 |
| 36 | | | | | | |
| TCR- | 287 | 487 | 289 | 290 | 488 | 489 |
| 37 | | | | | | |
| TCR- | 496 | 497 | 498 | 499 | 500 | 76 |
| 38 | | | | | | |
| TCR- | 507 | 508 | 509 | 510 | 511 | 54 |
| 39 | | | | | | |
| TCR- | 154 | 522 | 156 | 157 | 158 | 141 |
| 40 | | | | | | |
| TCR- | 534 | 535 | 536 | 537 | 538 | 539 |
| 41 | | | | | | |
| TCR- | 287 | 545 | 289 | 290 | 291 | 546 |
| 42 | | | | | | |
| TCR- | 553 | 554 | 555 | 556 | 557 | 76 |
| 43 | | | | | | |
| TCR- | 136 | 563 | 138 | 139 | 140 | 480 |
| 44 | | | | | | |
| TCR- | 136 | 569 | 138 | 139 | 140 | 18 |
| 45 | | | | | | |
| TCR- | 576 | 577 | 578 | 579 | 580 | 356 |
| 46 | | | | | | |
| TCR- | 119 | 586 | 121 | 122 | 123 | 587 |
| 47 | | | | | | |
| TCR- | 595 | 596 | 597 | 598 | 599 | 600 |
| 48 | | | | | | |
| TCR- | 576 | 606 | 578 | 579 | 580 | 607 |
| 49 | | | | | | |
| TCR- | 304 | 613 | 306 | 307 | 308 | 18 |
| 50 | | | | | | |
| TCR- | 451 | 619 | 453 | 454 | 455 | 489 |
| 51 | | | | | | |
| TCR- | 304 | 625 | 306 | 307 | 308 | 626 |
| 52 | | | | | | |
| TCR- | 595 | 636 | 597 | 598 | 599 | 292 |
| 53 | | | | | | |
| TCR- | 451 | 647 | 453 | 454 | 455 | 489 |
| 54 | | | | | | |
| TCR- | 315 | 653 | 317 | 318 | 319 | 94 |
| 55 | | | | | | |
| TCR- | 660 | 661 | 406 | 407 | 408 | 662 |
| 56 | | | | | | |
| TCR- | 674 | 675 | 676 | 677 | 678 | 339 |
| 57 | | | | | | |
| TCR- | 428 | 684 | 430 | 431 | 432 | 685 |
| 58 | | | | | | |
| TCR- | 119 | 691 | 121 | 122 | 123 | 392 |
| 59 | | | | | | |
| TCR- | 428 | 697 | 430 | 431 | 432 | 392 |
| 60 | | | | | | |
| TCR- | 595 | 703 | 597 | 598 | 599 | 18 |
| 61 | | | | | | |
| TCR- | 507 | 711 | 509 | 510 | 511 | 587 |
| 62 | | | | | | |
| TCR- | 208 | 717 | 210 | 211 | 212 | 363 |
| 63 | | | | | | |
| TCR- | 107 | 723 | 724 | 110 | 111 | 600 |
| 64 | | | | | | |
| TCR- | 119 | 730 | 121 | 122 | 123 | 456 |
| 65 | | | | | | |
| TCR- | 553 | 736 | 555 | 556 | 557 | 546 |
| 66 | | | | | | |
| TCR- | 208 | 742 | 210 | 374 | 212 | 546 |
| 67 | | | | | | |
| TCR- | 208 | 750 | 210 | 374 | 212 | 409 |
| 68 | | | | | | |
| TCR- | 451 | 756 | 453 | 454 | 455 | 242 |
| 69 | | | | | | |
| TCR- | 763 | 764 | 765 | 766 | 767 | 94 |
| 70 | | | | | | |
| TCR- | 315 | 773 | 317 | 318 | 319 | 774 |
| 71 | | | | | | |
| TCR- | 208 | 780 | 210 | 211 | 212 | 94 |
| 72 | | | | | | |
| TCR- | 119 | 786 | 121 | 122 | 123 | 787 |
| 73 | | | | | | |
| TCR- | 315 | 796 | 317 | 318 | 319 | 409 |
| 74 | | | | | | |
| TCR- | 451 | 802 | 453 | 454 | 455 | 141 |
| 75 | | | | | | |
| TCR- | 13 | 808 | 15 | 16 | 17 | 292 |
| 76.1 | | | | | | |
| TCR- | 287 | 811 | 289 | 290 | 291 | 812 |
| 76.2 | | | | | | |
| TCR- | 89 | 819 | 439 | 92 | 93 | 363 |
| 77 | | | | | | |
| TCR- | 404 | 825 | 406 | 407 | 408 | 392 |
| 78 | | | | | | |
| TCR- | 832 | 833 | 834 | 835 | 836 | 141 |
| 79 | | | | | | |
| TCR- | 763 | 842 | 765 | 766 | 767 | 546 |
| 80 | | | | | | |
| TCR- | 31 | 853 | 33 | 854 | 855 | 36 |
| 81 | | | | | | |
| TCR- | 674 | 866 | 676 | 677 | 678 | 867 |
| 82 | | | | | | |
| TCR- | 315 | 874 | 317 | 318 | 319 | 112 |
| 83 | | | | | | |
| TCR- | 304 | 880 | 306 | 307 | 308 | 662 |
| 84 | | | | | | |
| TCR- | 887 | 888 | 889 | 890 | 891 | 480 |
| 85 | | | | | | |
| TCR- | 208 | 902 | 210 | 211 | 212 | 903 |
| 86 | | | | | | |
| TCR- | 49 | 909 | 51 | 52 | 53 | 910 |
| 87 | | | | | | |
| TCR- | 507 | 916 | 509 | 510 | 511 | 456 |
| 88 | | | | | | |
| TCR- | 237 | 922 | 239 | 240 | 241 | 546 |
| 89 | | | | | | |
| TCR- | 534 | 928 | 536 | 537 | 538 | 230 |
| 90 | | | | | | |
| TCR- | 208 | 937 | 210 | 374 | 212 | 76 |
| 91.1 | | | | | | |
| TCR- | 208 | 937 | 210 | 374 | 212 | 76 |
| 91.2 | | | | | | |
| TCR- | 287 | 943 | 289 | 290 | 291 | 685 |
| 92 | | | | | | |
| TCR- | 208 | 949 | 210 | 374 | 212 | 112 |
| 93 | | | | | | |
| TCR- | 119 | 955 | 121 | 122 | 123 | 272 |
| 94 | | | | | | |
| TCR- | 208 | 961 | 210 | 211 | 212 | 36 |
| 95 | | | | | | |
| TCR- | 416 | 967 | 418 | 419 | 420 | 356 |
| 96 | | | | | | |
| TCR- | 974 | 975 | 976 | 977 | 978 | 867 |
| 97 | | | | | | |
| TCR- | 119 | 984 | 121 | 122 | 123 | 272 |
| 98 | | | | | | |
| TCR- | 71 | 990 | 73 | 74 | 75 | 94 |
| 99 | | | | | | |
| TCR- | 507 | 996 | 509 | 510 | 511 | 539 |
| 100 | | | | | | |
| TCR- | 119 | 1002 | 121 | 122 | 123 | 587 |
| 101 | | | | | | |
| TCR- | 507 | 1012 | 509 | 510 | 511 | 587 |
| 102 | | | | | | |
| TCR- | 13 | 1018 | 15 | 16 | 17 | 685 |
| 103 | | | | | | |
| TCR- | 428 | 684 | 430 | 431 | 432 | 685 |
| 104 | | | | | | |
| TCR- | 154 | 1024 | 156 | 157 | 158 | 1025 |
| 105 | | | | | | |
| TCR- | 832 | 1031 | 834 | 835 | 836 | 54 |
| 106 | | | | | | |
| TCR- | 304 | 1037 | 306 | 307 | 308 | 141 |
| 107 | | | | | | |
| TCR- | 404 | 1043 | 406 | 407 | 408 | 685 |
| 108 | | | | | | |
| TCR- | 507 | 1053 | 509 | 510 | 511 | 124 |
| 109 | | | | | | |
| TCR- | 534 | 1059 | 536 | 537 | 538 | 607 |
| 110 | | | | | | |
| TCR- | 107 | 1066 | 724 | 110 | 111 | 230 |
| 111 | | | | | | |
| TCR- | 1073 | 1074 | 1075 | 1076 | 1077 | 363 |
| 112 | | | | | | |
| TCR- | 674 | 1084 | 676 | 677 | 678 | 36 |
| 113 | | | | | | |
| TCR- | 13 | 1090 | 15 | 16 | 17 | 76 |
| 114 | | | | | | |
| TCR- | 553 | 1096 | 555 | 556 | 557 | 363 |
| 115 | | | | | | |
| TCR- | 71 | 1102 | 73 | 74 | 75 | 1103 |
| 116 | | | | | | |
| TCR- | 49 | 1109 | 51 | 52 | 53 | 339 |
| 117 | | | | | | |
| TCR- | 287 | 1116 | 289 | 290 | 488 | 607 |
| 118 | | | | | | |
| TCR- | 553 | 1122 | 555 | 556 | 557 | 141 |
| 119 | | | | | | |
| TCR- | 887 | 1133 | 889 | 890 | 891 | 1134 |
| 120 | | | | | | |
| TCR- | 49 | 1140 | 51 | 52 | 53 | 292 |
| 121 | | | | | | |
| TCR- | 119 | 1146 | 121 | 122 | 123 | 124 |
| 122 | | | | | | |
| TCR- | 107 | 1152 | 724 | 110 | 111 | 76 |
| 123 | | | | | | |
| TCR- | 119 | 1158 | 121 | 122 | 123 | 94 |
| 124 | | | | | | |
| TCR- | 553 | 1164 | 555 | 556 | 557 | 600 |
| 125 | | | | | | |
| TCR- | 107 | 1174 | 724 | 110 | 111 | 18 |
| 126 | | | | | | |
| TCR- | 451 | 1180 | 453 | 454 | 455 | 1181 |
| 127 | | | | | | |
| TCR- | 315 | 1187 | 317 | 318 | 319 | 600 |
| 128 | | | | | | |
| TCR- | 763 | 1193 | 765 | 766 | 767 | 320 |
| 129 | | | | | | |
| TCR- | 49 | 1199 | 51 | 52 | 53 | 489 |
| 130 | | | | | | |
| TCR- | 208 | 1205 | 210 | 374 | 212 | 292 |
| 131 | | | | | | |
| TCR- | 119 | 1211 | 121 | 122 | 123 | 112 |
| 132 | | | | | | |
| TCR- | 974 | 1217 | 976 | 977 | 978 | 124 |
| 133 | | | | | | |
| TCR- | 451 | 1223 | 453 | 454 | 455 | 356 |
| 134 | | | | | | |
| TCR- | 534 | 1229 | 536 | 537 | 538 | 94 |
| 135 | | | | | | |
| TCR- | 507 | 1235 | 509 | 510 | 511 | 272 |
| 136 | | | | | | |
| TCR- | 1073 | 1241 | 1075 | 1076 | 1077 | 94 |
| 137 | | | | | | |
| TCR- | 496 | 1247 | 498 | 499 | 1248 | 356 |
| 138 | | | | | | |
| TCR- | 208 | 1254 | 210 | 374 | 212 | 480 |
| 139 | | | | | | |
| TCR- | 225 | 1260 | 227 | 228 | 229 | 1261 |
| 140 | | | | | | |
| TCR- | 428 | 1267 | 430 | 431 | 432 | 190 |
| 141 | | | | | | |
| TCR- | 315 | 1273 | 317 | 318 | 319 | 242 |
| 142 | | | | | | |
| TCR- | 225 | 1279 | 227 | 228 | 229 | 292 |
| 143 | | | | | | |
| TCR- | 674 | 1285 | 676 | 677 | 678 | 112 |
| 144 | | | | | | |
| TCR- | 507 | 1291 | 509 | 510 | 511 | 480 |
| 145 | | | | | | |
| TCR- | 451 | 1297 | 453 | 454 | 455 | 356 |
| 146 | | | | | | |
| TCR- | 13 | 1303 | 15 | 130 | 17 | 1103 |
| 147 | | | | | | |
| TCR- | 674 | 1310 | 676 | 677 | 678 | 36 |
| 148 | | | | | | |
| TCR- | 13 | 1316 | 15 | 1304 | 17 | 18 |
| 149 | | | | | | |
| TCR- | 287 | 1322 | 289 | 290 | 291 | 685 |
| 150 | | | | | | |
| TCR- | 71 | 1328 | 73 | 74 | 75 | 18 |
| 151 | | | | | | |
| TCR- | 237 | 1334 | 239 | 240 | 241 | 774 |
| 152 | | | | | | |
| TCR- | 576 | 1340 | 578 | 579 | 580 | 489 |
| 153 | | | | | | |
| TCR- | 119 | 1346 | 121 | 122 | 123 | 489 |
| 154.1 | | | | | | |
| TCR- | 553 | 1349 | 555 | 556 | 557 | 1181 |
| 154.2 | | | | | | |
| TCR- | 507 | 508 | 509 | 510 | 511 | 54 |
| 155 | | | | | | |
| TCR- | 154 | 522 | 156 | 157 | 158 | 141 |
| 156 | | | | | | |
| TCR- | 534 | 535 | 536 | 537 | 538 | 539 |
| 157 | | | | | | |
| TCR- | 287 | 545 | 289 | 290 | 291 | 546 |
| 158 | | | | | | |
| TCR- | 553 | 554 | 555 | 556 | 557 | 76 |
| 159 | | | | | | |
| TCR- | 136 | 563 | 138 | 139 | 140 | 480 |
| 160 | | | | | | |
| TCR- | 136 | 569 | 138 | 139 | 140 | 18 |
| 161 | | | | | | |
| TCR- | 576 | 577 | 578 | 579 | 580 | 356 |
| 162 | | | | | | |
| TCR- | 119 | 586 | 121 | 122 | 123 | 587 |
| 163 | | | | | | |
| TCR- | 595 | 596 | 597 | 598 | 599 | 600 |
| 164 | | | | | | |
| TCR- | 576 | 606 | 578 | 579 | 580 | 607 |
| 165 | | | | | | |
| TCR- | 304 | 613 | 306 | 307 | 308 | 18 |
| 166 | | | | | | |
| TCR- | 451 | 619 | 453 | 454 | 455 | 489 |
| 167 | | | | | | |
| TCR- | 304 | 625 | 306 | 307 | 308 | 626 |
| 168 | | | | | | |
| TCR- | 595 | 636 | 597 | 598 | 599 | 292 |
| 169 | | | | | | |
| TCR- | 451 | 647 | 453 | 454 | 455 | 489 |
| 170 | | | | | | |
| TCR- | 315 | 653 | 317 | 318 | 319 | 94 |
| 171 | | | | | | |
| TCR- | 660 | 661 | 406 | 407 | 408 | 662 |
| 172 | | | | | | |
| TCR- | 674 | 675 | 676 | 677 | 678 | 339 |
| 173 | | | | | | |
| TCR- | 428 | 684 | 430 | 431 | 432 | 685 |
| 174 | | | | | | |
| TCR- | 119 | 691 | 121 | 122 | 123 | 392 |
| 175 | | | | | | |
| TCR- | 428 | 697 | 430 | 431 | 432 | 392 |
| 176 | | | | | | |
| TCR- | 595 | 703 | 597 | 598 | 599 | 18 |
| 177 | | | | | | |
| TCR- | 507 | 711 | 509 | 510 | 511 | 587 |
| 178 | | | | | | |
| TCR- | 208 | 717 | 210 | 211 | 212 | 363 |
| 179 | | | | | | |
| TCR- | 208 | 780 | 210 | 211 | 212 | 94 |
| 180 | | | | | | |
| TCR- | 119 | 786 | 121 | 122 | 123 | 787 |
| 181 | | | | | | |
| TCR- | 315 | 793 | 317 | 318 | 319 | 409 |
| 182 | | | | | | |
| TCR- | 451 | 802 | 453 | 454 | 455 | 141 |
| 183 | | | | | | |
| TCR- | 13 | 808 | 15 | 16 | 17 | 292 |
| 184.1 | | | | | | |
| TCR- | 287 | 811 | 289 | 290 | 291 | 812 |
| 184.2 | | | | | | |
| TCR- | 89 | 819 | 439 | 92 | 93 | 363 |
| 185 | | | | | | |
| TCR- | 404 | 825 | 406 | 407 | 408 | 392 |
| 186 | | | | | | |
| TCR- | 832 | 833 | 834 | 835 | 836 | 141 |
| 187 | | | | | | |
| TCR- | 49 | 909 | 51 | 52 | 53 | 910 |
| 188 | | | | | | |
| TCR- | 507 | 916 | 509 | 510 | 511 | 456 |
| 189 | | | | | | |
| TCR- | 237 | 922 | 239 | 240 | 241 | 546 |
| 190 | | | | | | |
| TCR- | 534 | 928 | 536 | 537 | 538 | 230 |
| 191 | | | | | | |
| TCR- | 208 | 937 | 210 | 374 | 212 | 76 |
| 192.1 | | | | | | |
| TCR- | 208 | 937 | 210 | 374 | 212 | 76 |
| 192.2 | | | | | | |
| TCR- | 287 | 943 | 289 | 290 | 291 | 685 |
| 193 | | | | | | |
| TCR- | 428 | 684 | 430 | 431 | 432 | 685 |
| 194 | | | | | | |
| TCR- | 154 | 1024 | 156 | 157 | 158 | 1025 |
| 195 | | | | | | |
| TCR- | 832 | 1031 | 834 | 835 | 836 | 54 |
| 196 | | | | | | |
| TCR- | 304 | 1037 | 306 | 307 | 308 | 141 |
| 197 | | | | | | |
| TCR- | 404 | 1043 | 406 | 407 | 408 | 685 |
| 198 | | | | | | |
| TCR- | 507 | 1053 | 509 | 510 | 511 | 124 |
| 199 | | | | | | |
| TCR- | 534 | 1059 | 536 | 537 | 538 | 607 |
| 200 | | | | | | |
| TCR- | 107 | 1066 | 724 | 110 | 111 | 230 |
| 201 | | | | | | |
| TCR- | 1073 | 1074 | 1075 | 1076 | 1077 | 363 |
| 202 | | | | | | |
| TCR- | 674 | 1084 | 676 | 677 | 678 | 36 |
| 203 | | | | | | |
| TCR- | 13 | 1090 | 15 | 16 | 17 | 76 |
| 204 | | | | | | |
| TCR- | 553 | 1096 | 555 | 556 | 557 | 363 |
| 205 | | | | | | |
| TCR- | 71 | 1102 | 73 | 74 | 75 | 1103 |
| 206 | | | | | | |
| TCR- | 49 | 1109 | 51 | 52 | 53 | 339 |
| 207 | | | | | | |
| TCR- | 287 | 1116 | 289 | 290 | 488 | 607 |
| 208 | | | | | | |
| TCR- | 553 | 1122 | 555 | 556 | 557 | 141 |
| 209 | | | | | | |
| TCR- | 887 | 1133 | 889 | 890 | 891 | 1134 |
| 210 | | | | | | |
| TCR- | 49 | 1140 | 51 | 52 | 53 | 292 |
| 211 | | | | | | |
| TCR- | 119 | 1146 | 121 | 122 | 123 | 124 |
| 212 | | | | | | |
| TCR- | 428 | 1267 | 430 | 431 | 432 | 190 |
| 213 | | | | | | |
| TCR- | 315 | 1273 | 317 | 318 | 319 | 242 |
| 214 | | | | | | |
| TCR- | 225 | 1279 | 227 | 228 | 229 | 292 |
| 215 | | | | | | |
| TCR- | 674 | 1285 | 676 | 677 | 678 | 112 |
| 216 | | | | | | |
| TCR- | 507 | 1291 | 509 | 510 | 511 | 480 |
| 217 | | | | | | |
| TCR- | 451 | 1297 | 453 | 454 | 455 | 356 |
| 218 | | | | | | |
| TCR- | 13 | 1303 | 15 | 1304 | 17 | 1103 |
| 219 | | | | | | |
| TCR- | 674 | 1310 | 676 | 677 | 678 | 36 |
| 220 | | | | | | |
| TCR- | 13 | 1316 | 15 | 1304 | 17 | 18 |
| 221 | | | | | | |
| TCR- | 287 | 1322 | 289 | 290 | 291 | 685 |
| 222 | | | | | | |

### II. Proteinaceous Compositions

As used herein, a “protein” “peptide” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS:1-1359. In specific aspects, the peptide or polypeptide is or is based on a human sequence. In certain aspects, the peptide or polypeptide is not naturally occurring and/or is in a combination of peptides or polypeptides.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOS:1-1359 (or any derivable range therein) and may be a substitution with any amino acid or may be a substitution with a alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS:1-1359.

In some aspects, the protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS:1-1359 and have or have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOS:1-1359.

In some aspects, the protein, polypeptide, or nucleic acid may comprise, comprise at least, or comprise at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids or nucleic acids of SEQ ID NOS:1-1359.

In some aspects, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids of SEQ ID NOS:1-1359 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOS:1-1359.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 of any of SEQ ID NOS:1-1359 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 (or any derivable range therein) contiguous amino acids or nucleotides of any of SEQ ID NOS:1-1359.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

In some aspects of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### III. Nucleic Acids

In certain aspects, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another aspect, the nucleic acid molecules may be used as probes or PCR primers for specific antibody sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In a preferred aspect, the nucleic acid molecules are oligonucleotides. In a more preferred aspect, the oligonucleotides are from highly variable regions of the heavy and light or alpha and beta chains of the antibody or TCR of interest. In an even more preferred aspect, the oligonucleotides encode all or part of one or more of the CDRs or TCRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### IV. Polypeptide Expression

In some aspects, there are nucleic acid molecule encoding polypeptides or peptides of the disclosure (e.g TCR genes). These may be generated by methods known in the art, e.g., isolated from B cells of mice that have been immunized and isolated, phage display, expressed in any suitable recombinant expression system and allowed to assemble to form antibody molecules or by recombinant methods.

### A. Expression

The nucleic acid molecules may be used to express large quantities of polypeptides. If the nucleic acid molecules are derived from a non-human, non-transgenic animal, the nucleic acid molecules may be used for humanization of the TCR genes.

### B. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, alpha chain, beta chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin or TCR sequence with appropriate restriction sites engineered so that any variable region sequences can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### C. Expression Systems

Numerous expression systems exist that comprise at least a part or all of the expression vectors discussed above. Prokaryote- and/or eukaryote-based systems can be employed for use with an aspect to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### V. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### A. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy, light, alpha, and beta chains of an antibody or TCR, or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### VI. Additional Therapies

### A. Immunotherapy

In some aspects, the methods comprise administration of an additional therapy. In some aspects, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### 1. Checkpoint Inhibitors and Combination Treatment

Aspects of the disclosure may include administration of immune checkpoint inhibitors, which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include Bf7-H1, B7-4, CD274, and B37-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 2. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### B. Chemotherapies

In some aspects, the additional therapy comprises a chemotherapy. Suitable classes of chemotherapeutic agents include (a) Alkylating Agents, such as nitrogen mustards (e.g., mechlorethamine, cyclophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozotocin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophyllotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydrazine derivatives (e.g., procarbazine), and adrenocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### C. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

### D. Surgery

In some aspects, the additional therapy comprises surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months (or any range derivable therein). These treatments may be of varying dosages as well.

### VII. Detection and Therapeutic Agents

In some aspects of this disclosure, it will be useful to detectably or therapeutically label the TCRs or fusion proteins of the disclosure. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

The conjugated agents can be linked to the polypeptide directly or indirectly, using any of a large number of available methods. For example, an agent can be attached at the hinge region of the reduced antibody component via disulfide bond formation, using cross-linkers such as N-succinyl 3-(2-pyridyldithio)proprionate (SPDP), or via a carbohydrate moiety in the Fc region of the antibody (Yu et al., 1994; Upeslacis et al., 1995; Price, 1995).

Techniques for conjugating agents to polypeptides are well known (Amon et al., 1985; Hellstrom et al., 1987; Thorpe, 1985; Baldwin et al., 1985; Thorpe et al., 1982),

The polypeptides of the disclosure or antigen-binding regions thereof can be linked to another functional molecule such as ligands, cytotoxic molecules, chemotherapeutic agents, or other agents described as additional therapeutics.

### VIII. Formulations and Culture of the Cells

In particular aspects, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolglycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain aspects, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific aspects, one or more of these may be explicitly excluded.

In some aspects, the medium further comprises vitamins. In some aspects, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some aspects, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some aspects, the vitamins include or consist essentially of biotin, DL alpha tocopherol acetate, DL alpha-tocopherol, vitamin A, or combinations or salts thereof. In some aspects, the medium further comprises proteins. In some aspects, the proteins comprise albumin or bovine serum albumin, a fraction of BSA, catalase, insulin, transferrin, superoxide dismutase, or combinations thereof. In some aspects, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some aspects, the medium comprises one or more of the following: a B-27® supplement, xeno-free B-27® supplement, GS21TM supplement, or combinations thereof. In some aspects, the medium comprises or further comprises amino acids, monosaccharides, inorganic ions. In some aspects, the amino acids comprise arginine, cystine, isoleucine, leucine, lysine, methionine, glutamine, phenylalanine, threonine, tryptophan, histidine, tyrosine, or valine, or combinations thereof. In some aspects, the inorganic ions comprise sodium, potassium, calcium, magnesium, nitrogen, or phosphorus, or combinations or salts thereof. In some aspects, the medium further comprises one or more of the following: molybdenum, vanadium, iron, zinc, selenium, copper, or manganese, or combinations thereof. In certain aspects, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific aspects, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific aspects, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, pg/ml, mg/ml, or any range derivable therein.

In specific aspects, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular aspects the cells are in a formulated cell suspension that is stable at room temperature for 1, 2, 3, or 4 hours or more from time of thawing.

In particular aspects, the cells of the disclosure comprise an exogenous TCR, which may be of a defined antigen specificity. In some aspects, the TCR can be selected based on absent or reduced alloreactivity to the intended recipient (examples include certain virus-specific TCRs, xeno-specific TCRs, or cancer-testis antigen-specific TCRs). In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some aspects, the choice of exogenous TCR may not necessarily be defined based on lack of alloreactivity. In some aspects, the endogenous TCR genes have been modified by genome editing so that they do not express a protein. Methods of gene editing such as methods using the CRISPR/Cas9 system are known in the art and described herein.

In some aspects, the cells of the disclosure further comprise one or more chimeric antigen receptors (CARs). Examples of tumor cell antigens to which a CAR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FRα, GD2, G250/CAIX, GD3, Glypican-3 (GPC3), Her2, IL-13Rα2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### IX. Administration of Therapeutic Compositions

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic compositions of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

The cancers amenable for treatment include, but are not limited to, tumors of all types, locations, sizes, and characteristics. In some aspects, the cancer comprises a solid tumor. In some aspects, the methods relate to reducing tumor volume or treating cancers that are recurrent and/or metastatic. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, glioblastoma, glioblatoma multiforme, supratentorial primitive neuroectodermal tumors brain tumor, visual pathway and hypothalamic glioma, breast cancer, lymphoid cancer, bronchial adenomas/carcinoids, tracheal cancer, lung cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### X. Kits

Certain aspects of the present invention also concern kits containing compositions of the disclosure or compositions to implement methods of the invention. In some aspects, kits can be used to evaluate one or more polypeptides or HLA types. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

In certain aspects, negative and/or positive control nucleic acids, probes, and inhibitors are included in some kit aspects. In addition, a kit may include a sample that is a negative or positive control for methylation of one or more biomarkers.

It is contemplated that any method or composition described herein can be implemented with respect to any other method or composition described herein and that different aspects may be combined. The claims originally filed are contemplated to cover claims that are multiply dependent on any filed claim or combination of filed claims.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. All references cited herein are specifically incorporated by reference for all purposes.

What is claimed is:

1. A polypeptide comprising an antigen binding variable region comprising a complementarity determining region (CDR) 3 comprising the amino acid sequence of a CDR3-b identified from a T-cell receptor (TCR) clone in Table 1 or an amino acid sequence with at least 80% sequence identity to the amino acid sequence of a CDR3-b from a TCR clone in Table 1.

2. The polypeptide of claim 1, wherein the variable region comprises a CDR1, CDR2, and/or CDR3.

3. The polypeptide of claim 2, wherein the variable region comprises a CDR1 with the amino acid sequence of the CDR1-b from the corresponding TCR clone in Table 1 or an amino acid sequence with at least 80% sequence identity to the CDR1-b from the corresponding TCR clone in Table 1.

4. The polypeptide of claim 2 or 3, wherein the variable region comprises a CDR2 with the amino acid sequence of the CDR2-b from the corresponding TCR clone in Table 1 or an amino acid sequence with at least 80% sequence identity to the CDR2-b from the corresponding TCR clone in Table 1.

5. The polypeptide of claim 3 or 4, wherein the variable region comprises a CDR1, CDR2, and CDR3 with an amino acid sequence of a CDR1-b, CDR2-b, and CDR3-b from the corresponding TCR clone in Table 1.

6. The polypeptide of any one of claims 1-5, wherein the variable region comprises the amino acid sequence of the variable-b from the corresponding TCR clone in Table 1 or an amino acid sequence with at least 80% sequence identity to the variable-b from the corresponding TCR clone in Table 1.

7. The polypeptide of any one of claims 1-6, wherein the polypeptide comprises a T cell receptor beta (TCR-b) variable region from the corresponding TCR clone in Table 1.

8. The polypeptide of any one of claims 1-7, wherein the polypeptide comprises a TCR-b variable and constant region.

9. A polypeptide comprising an antigen binding variable region comprising a complementarity determining region (CDR) 3 comprising the amino acid sequence of a CDR3-a identified from a T-cell receptor (TCR) clone in Table 1 or an amino acid sequence with at least 80% sequence identity to the amino acid sequence of a CDR3-a from a TCR clone in Table 1.

10. The polypeptide of claim 9, wherein the variable region comprises a CDR1, CDR2, and/or CDR3.

11. The polypeptide of claim 10, wherein the variable region comprises a CDR1 with the amino acid sequence of the CDR1-a from the corresponding TCR clone in Table 1 or an amino acid sequence with at least 80% sequence identity to the CDR1-a from the corresponding TCR clone in Table 1.

12. The polypeptide of claim 10 or 11, wherein the variable region comprises a CDR2 with the amino acid sequence of the CDR2-a from the corresponding TCR clone in Table 1 or an amino acid sequence with at least 80% sequence identity to the CDR2-a from the corresponding TCR clone in Table 1.

13. The polypeptide of claim 11 or 12, wherein the variable region comprises a CDR1, CDR2, and CDR3 with an amino acid sequence of a CDR1-a, CDR2-a, and CDR3-a from the corresponding TCR clone of Table 1.

14. The polypeptide of any one of claims 9-13, wherein the variable region comprises the amino acid sequence of the variable-a from the corresponding TCR clone in Table 1 or an amino acid sequence with at least 80% sequence identity to the variable-a from the corresponding TCR clone in Table 1.

15. The polypeptide of any one of claims 9-14, wherein the polypeptide comprises a TCR-a variable and constant region.

16. The polypeptide of any one of claims 1-15, wherein the polypeptide further comprises a signal peptide.

17. An engineered TCR comprising a TCR-b polypeptide and a TCR-a polypeptide, wherein the TCR-b polypeptide comprises a CDR3 comprising the amino acid sequence of a CDR3-b identified from a TCR clone in Table 1 or an amino acid sequence with at least 80% sequence identity to the amino acid sequence of a CDR3-b from a TCR clone identified in Table 1 and the TCR-a polypeptide comprises a CDR3 comprising the amino acid sequence of a CDR3-a identified from the corresponding TCR clone in Table 1 or an amino acid sequence with at least 80% sequence identity to the amino acid sequence of a CDR3-a from the corresponding TCR clone identified in Table 1.

18. The TCR of claim 17, wherein the TCR comprises a TCR-b polypeptide comprising a variable region comprising CDR1, CDR2, and CDR3 and a TCR-a polypeptide comprising a variable region comprising CDR1, CDR2, and CDR3.

19. The TCR of claim 18, wherein the TCR-b polypeptide comprises a CDR1 comprising the amino acid sequence of a CDR1-b of the corresponding TCR clone in Table 1 or an amino acid sequence with at least 80% sequence identity to a CDR1-b of the corresponding TCR clone in Table 1 and/or the TCR-a polypeptide comprises a CDR1-a of the corresponding TCR clone in Table 1 or an amino acid sequence with at least 80% sequence identity to a CDR1-a of the corresponding TCR clone in Table 1.

20. The TCR of any one of claims 18-19, wherein the TCR-b polypeptide comprises a CDR2 with the amino acid sequence of the CDR2-b from the corresponding TCR clone in Table 1 or an amino acid sequence with at least 80% sequence identity to the CDR2-b from the corresponding TCR clone in Table 1 and the TCR-a polypeptide comprises a CDR2 with the amino acid sequence of the CDR2-b from the corresponding TCR clone in Table 1 or an amino acid sequence with at least 80% sequence identity to the CDR2-b from the corresponding TCR clone in Table 1.

21. The TCR of any one of claims 18-20, wherein the CDR1, CDR2, and CDR3 of the TCR-b polypeptide comprise the amino acid sequence of a CDR1-b, CDR2-b, and CDR3-b from a TCR clone of Table 1 and the CDR1, CDR3, and CDR3 of the TCR-a polypeptide comprise the amino acid sequence of the CDR1-a, CDR2-a, and CDR3-a from the corresponding TCR clone in Table 1.

22. The TCR of any one of claims 18-21, wherein the TCR-b polypeptide comprises an amino acid sequence with at least 70% sequence identity to the variable-b from the corresponding TCR clone in Table 1 and the TCR-a polypeptide comprises an amino acid sequence with at least 70% sequence identity to the variable-a from the same TCR clone in Table 1.

23. The TCR of claim 22, wherein the TCR-b polypeptide comprises the amino acid sequence the variable-b from the corresponding TCR clone in Table 1 and the TCR-a polypeptide comprises the amino acid sequence the variable-b from the corresponding TCR clone in Table 1.

24. The TCR of any one of claims 17-23, wherein the TCR comprises a modification or is chimeric.

25. The TCR of any one of claims 17-24, wherein the TCR-a polypeptide and TCR-b polypeptide are operably linked.

26. The TCR of claim 25, wherein the TCR-a polypeptide and TCR-b polypeptide are operably linked through a peptide bond.

27. The TCR of claim 25, wherein the TCR is a single chain TCR.

28. The TCR of claim 26 or 28, wherein the TCR-a polypeptide and TCR-b polypeptide are on the same polypeptide and wherein the TCR-b is amino-proximal to the TCR-a.

29. The TCR of claim 26 or 28, wherein the TCR-a polypeptide and TCR-b polypeptide are on the same polypeptide and wherein the TCR-a is amino-proximal to the TCR-b.

30. The TCR of any one of claims 26-29, wherein the TCR comprises a linker between the TCR-a and TCR-b polypeptide.

31. The TCR of one of claims 26-30, wherein the linker comprises glycine and serine residues.

32. A fusion protein comprising the TCR of any one of claims 17-31 and a CD3 binding region.

33. The fusion protein of claim 32, wherein the CD3 binding region comprises a CD3-specific fragment antigen binding (Fab), single chain variable fragment (scFv), single domain antibody, or single chain antibody.

34. The TCR of any one of claims 17-31 or the fusion protein of claim 32 or 33, wherein the TCR or fusion protein is conjugated to a detection or therapeutic agent.

35. The TCR or fusion protein of claim 34, wherein the agent comprises a fluorescent molecule, radiative molecule, or toxin.

36. A nucleic acid encoding the polypeptide of any one of claims 1-16, the TCR of any one of claims 17-31 or 34-35, or the fusion protein of any one of claims 32-35.

37. The nucleic acid of claim 36, wherein the nucleic acid is RNA.

38. The nucleic acid of claim 36, wherein the nucleic acid is DNA or a cDNA encoding the polypeptide or a complement of the polypeptide.

39. A nucleic acid expression vector comprising the nucleic acid(s) of any one of claims 36-38.

40. The vector of claim 39, wherein the vector comprises a promoter that directs the expression of the nucleic acid.

41. The vector of claim 39 or 40, wherein the vector comprises the TCR-a and TCR-b genes.

42. A cell comprising the polypeptide of any one of claims 1-16, the TCR of any one of claims 17-31 or 34-35, the fusion protein of any one of claims 32-35, the nucleic acid(s) of any one of claims 36-38, or the vector of any one of claims 39-41.

43. The cell of claim 42, wherein the cell comprises a stem cell, a progenitor cell, an immune cell, or a natural killer (NK) cell.

44. The cell of claim 43, wherein the cell comprises a hematopoietic stem or progenitor cell, a T cell, a cell differentiated from mesenchymal stem cells (MSCs) or an induced pluripotent stem cell (iPSC).

45. The cell of claim 43 or 44, wherein the cell is isolated or derived from peripheral blood mononuclear cell (PBMCs).

46. The cell of claim 44 or 45, wherein the T cell comprises a cytotoxic T lymphocyte (CTL), a CD8⁺ T cell, a CD4⁺ T cell, an invariant NK T (iNKT) cell, a gamma-delta T cell, a NKT cell, or a regulatory T cell.

47. The cell of any one of claims 42-46, wherein the cell is isolated from a cancer patient.

48. The cell of claim 47, wherein the cancer patient has glioblastoma multiforme.

49. A composition comprising the polypeptide of any one of claims 1-16, the TCR of any one of claims 17-31 or 34-35, the fusion protein of any one of claims 32-35, the nucleic acid(s) of any one of claims 36-38, the vector of any one of claims 39-41, or the cell of any one of claims 42-48.

50. The composition of claim 49, wherein the composition is formulated for parenteral administration, intravenous injection, intramuscular injection, inhalation, or subcutaneous injection.

51. The composition of any one of claims 49 or 50, wherein the composition is formulated as a vaccine.

52. The composition of any one of claims 49-51, wherein the composition further comprises an adjuvant.

53. The composition of any one of claims 49-52, wherein the composition has been determined to be serum-free, mycoplasma-free, endotoxin-free, and/or sterile.

54. A method of making an engineered cell comprising transferring the nucleic acid(s) of any one of claims 36-38 or the vector of any one of claims 39-41 into a cell.

55. The method of claim 54, wherein the method further comprises culturing the cell in media, incubating the cell at conditions that allow for the division of the cell, screening the cell, and/or freezing the cell.

56. A method of making a polypeptide comprising expressing the nucleic acid(s) of any one of claims 36-38 or the vector of any one of claims 39-41 in a cell.

57. The method of claim 56, wherein the method further comprises isolating the expressed polypeptide.

58. A method for treating or preventing cancer in a subject comprising administering the composition of any one of claims 49-55 or the cells of any one of claims 42-48 to a subject in need thereof.

59. A method of stimulating an immune response in a subject, the method comprising administering the composition of any one of claims 49-55 or the cells of any one of claims 42-48 to a subject in need thereof.

60. The method of claim 58, wherein the cancer comprises glioblastoma multiforme.

61. The method of claim 58, wherein the cancer comprises metastatic melanoma, melanoma, renal cell carcinoma, colorectal cancer, hepatocellular carcinoma, non-small cell lung cancer, malignant pleural mesothelioma, Hodgkin's lymphoma, head and neck cancer, urothelial carcinoma, small cell lung cancer, esophageal carcinoma, gastric cancer, cervical cancer, Merkel cell carcinoma, endometrial cancer, squamous cell carcinoma, bladder cancer, breast cancer, or basal cell carcinoma.

62. The method of claim 59, wherein the subject has cancer.

63. The method of claim 62, wherein the cancer comprises glioblastoma multiforme.

64. The method of claim 62, wherein the cancer comprises metastatic melanoma, melanoma, renal cell carcinoma, colorectal cancer, hepatocellular carcinoma, non-small cell lung cancer, malignant pleural mesothelioma, Hodgkin's lymphoma, head and neck cancer, urothelial carcinoma, small cell lung cancer, esophageal carcinoma, gastric cancer, cervical cancer, Merkel cell carcinoma, endometrial cancer, squamous cell carcinoma, bladder cancer, breast cancer, or basal cell carcinoma.

65. The method of any one of claims 58-64, wherein the subject is a human subject.

66. The method of any one of claims 58-65, wherein the cells are autologous.

67. The method of any one of claims 58-65, wherein the cells are allogenic.

68. The method of any one of claims 58-67, wherein the subject has previously been treated for the cancer.

69. The method of claim 68, wherein the subject has been determined to be resistant to the previous treatment.

70. The method of any one of claims 58-69, wherein the method further comprises the administration of an additional therapy.

71. The method of any one of claims 58-70, wherein the cancer comprises stage I, II, III, or IV cancer.

72. The method of any one of claims 58-71, wherein the cancer comprises metastatic and/or recurrent cancer.
